SEQUENCE LISTING (1449 sequences)

SEQ ID NO: 1 - DNA, 25 bp, Artificial Sequence
  Description of Artificial Sequence Synthetic oligonucleotide
ggagggatacctgtatattacaagt

SEQ ID NO: 2 - DNA, 25 bp, Artificial Sequence
  Description of Artificial Sequence Synthetic oligonucleotide
aggagggatacctgtatattacaag

SEQ ID NO: 3 - DNA, 25 bp, Artificial Sequence
  Description of Artificial Sequence Synthetic oligonucleotide
caggagggatacctgtatattacaa

SEQ ID NO: 4 - DNA, 25 bp, Artificial Sequence
  Description of Artificial Sequence Synthetic oligonucleotide
ccaggagggatacctgtatattaca

SEQ ID NO: 5 - DNA, 25 bp, Artificial Sequence
  Description of Artificial Sequence Synthetic oligonucleotide
accaggagggatacctgtatattac

SEQ ID NO: 6 - DNA, 25 bp, Artificial Sequence
  Description of Artificial Sequence Synthetic oligonucleotide
taccaggagggatacctgtatatta

SEQ ID NO: 7 - DNA, 25 bp, Artificial Sequence
  Description of Artificial Sequence Synthetic oligonucleotide
ttaccaggagggatacctgtatatt

SEQ ID NO: 8 - DNA, 25 bp, Artificial Sequence
  Description of Artificial Sequence Synthetic oligonucleotide
cttaccaggagggatacctgtatat

SEQ ID NO: 9 - DNA, 25 bp, Artificial Sequence
  Description of Artificial Sequence Synthetic oligonucleotide
gcttaccaggagggatacctgtata

SEQ ID NO: 10 - DNA, 25 bp, Artificial Sequence
  Description of Artificial Sequence Synthetic oligonucleotide
agcttaccaggagggatacctgtat

SEQ ID NO: 11 - DNA, 25 bp, Artificial Sequence
  Description of Artificial Sequence Synthetic oligonucleotide
gagcttaccaggagggatacctgta

SEQ ID NO: 12 - DNA, 25 bp, Artificial Sequence
  Description of Artificial Sequence Synthetic oligonucleotide
agagcttaccaggagggatacctgt

SEQ ID NO: 13 - DNA, 25 bp, Artificial Sequence
  Description of Artificial Sequence Synthetic oligonucleotide
cagagcttaccaggagggatacctg

SEQ ID NO: 14 - DNA, 25 bp, Artificial Sequence
  Description of Artificial Sequence Synthetic oligonucleotide
ccagagcttaccaggagggatacct

SEQ ID NO: 15 - DNA, 25 bp, Artificial Sequence
  Description of Artificial Sequence Synthetic oligonucleotide
accagagcttaccaggagggatacc

SEQ ID NO: 16 - DNA, 25 bp, Artificial Sequence
  Description of Artificial Sequence Synthetic oligonucleotide
taccagagcttaccaggagggatac

SEQ ID NO: 17 - DNA, 25 bp, Artificial Sequence
  Description of Artificial Sequence Synthetic oligonucleotide
ataccagagcttaccaggagggata

SEQ ID NO: 18 - DNA, 25 bp, Artificial Sequence
  Description of Artificial Sequence Synthetic oligonucleotide
aataccagagcttaccaggagggat

SEQ ID NO: 19 - DNA, 25 bp, Artificial Sequence
  Description of Artificial Sequence Synthetic oligonucleotide
taataccagagcttaccaggaggga

SEQ ID NO: 20 - DNA, 25 bp, Artificial Sequence
  Description of Artificial Sequence Synthetic oligonucleotide
ataataccagagcttaccaggaggg

SEQ ID NO: 21 - DNA, 25 bp, Artificial Sequence
  Description of Artificial Sequence Synthetic oligonucleotide
cataataccagagcttaccaggagg

SEQ ID NO: 22 - DNA, 25 bp, Artificial Sequence
  Description of Artificial Sequence Synthetic oligonucleotide
acataataccagagcttaccaggag

SEQ ID NO: 23 - DNA, 25 bp, Artificial Sequence
  Description of Artificial Sequence Synthetic oligonucleotide
gacataataccagagcttaccagga

SEQ ID NO: 24 - DNA, 25 bp, Artificial Sequence
  Description of Artificial Sequence Synthetic oligonucleotide
agacataataccagagcttaccagg

SEQ ID NO: 25 - DNA, 25 bp, Artificial Sequence
  Description of Artificial Sequence Synthetic oligonucleotide
aagacataataccagagcttaccag

SEQ ID NO: 26 - DNA, 25 bp, Artificial Sequence
  Description of Artificial Sequence Synthetic oligonucleotide
taagacataataccagagcttacca

SEQ ID NO: 27 - DNA, 25 bp, Artificial Sequence
  Description of Artificial Sequence Synthetic oligonucleotide
ttaagacataataccagagcttacc

SEQ ID NO: 28 - DNA, 25 bp, Artificial Sequence
  Description of Artificial Sequence Synthetic oligonucleotide
gttaagacataataccagagcttac

SEQ ID NO: 29 - DNA, 25 bp, Artificial Sequence
  Description of Artificial Sequence Synthetic oligonucleotide
tgttaagacataataccagagctta

SEQ ID NO: 30 - DNA, 25 bp, Artificial Sequence
  Description of Artificial Sequence Synthetic oligonucleotide
atgttaagacataataccagagctt

SEQ ID NO: 31 - DNA, 25 bp, Artificial Sequence
  Description of Artificial Sequence Synthetic oligonucleotide
aatgttaagacataataccagagct

SEQ ID NO: 32 - DNA, 25 bp, Artificial Sequence
  Description of Artificial Sequence Synthetic oligonucleotide
aaatgttaagacataataccagagc

SEQ ID NO: 33 - DNA, 25 bp, Artificial Sequence
  Description of Artificial Sequence Synthetic oligonucleotide
aaaatgttaagacataataccagag

SEQ ID NO: 34 - DNA, 25 bp, Artificial Sequence
  Description of Artificial Sequence Synthetic oligonucleotide
aaaaatgttaagacataataccaga

SEQ ID NO: 35 - DNA, 25 bp, Artificial Sequence
  Description of Artificial Sequence Synthetic oligonucleotide
taaaaatgttaagacataataccag

SEQ ID NO: 36 - DNA, 25 bp, Artificial Sequence
  Description of Artificial Sequence Synthetic oligonucleotide
ttaaaaatgttaagacataatacca

SEQ ID NO: 37 - DNA, 25 bp, Artificial Sequence
  Description of Artificial Sequence Synthetic oligonucleotide
tttaaaaatgttaagacataatacc

SEQ ID NO: 38 - DNA, 25 bp, Artificial Sequence
  Description of Artificial Sequence Synthetic oligonucleotide
atttaaaaatgttaagacataatac

SEQ ID NO: 39 - DNA, 25 bp, Artificial Sequence
  Description of Artificial Sequence Synthetic oligonucleotide
gatttaaaaatgttaagacataata

SEQ ID NO: 40 - DNA, 25 bp, Artificial Sequence
  Description of Artificial Sequence Synthetic oligonucleotide
agatttaaaaatgttaagacataat

SEQ ID NO: 41 - DNA, 25 bp, Artificial Sequence
  Description of Artificial Sequence Synthetic oligonucleotide
tagatttaaaaatgttaagacataa

SEQ ID NO: 42 - DNA, 25 bp, Artificial Sequence
  Description of Artificial Sequence Synthetic oligonucleotide
atagatttaaaaatgttaagacata

SEQ ID NO: 43 - DNA, 25 bp, Artificial Sequence
  Description of Artificial Sequence Synthetic oligonucleotide
catagatttaaaaatgttaagacat

SEQ ID NO: 44 - DNA, 25 bp, Artificial Sequence
  Description of Artificial Sequence Synthetic oligonucleotide
ccatagatttaaaaatgttaagaca

SEQ ID NO: 45 - DNA, 25 bp, Artificial Sequence
  Description of Artificial Sequence Synthetic oligonucleotide
accatagatttaaaaatgttaagac

SEQ ID NO: 46 - DNA, 25 bp, Artificial Sequence
  Description of Artificial Sequence Synthetic oligonucleotide
taccatagatttaaaaatgttaaga

SEQ ID NO: 47 - DNA, 25 bp, Artificial Sequence
  Description of Artificial Sequence Synthetic oligonucleotide
ttaccatagatttaaaaatgttaag

SEQ ID NO: 48 - DNA, 25 bp, Artificial Sequence
  Description of Artificial Sequence Synthetic oligonucleotide
attaccatagatttaaaaatgttaa

SEQ ID NO: 49 - DNA, 25 bp, Artificial Sequence
  Description of Artificial Sequence Synthetic oligonucleotide
gattaccatagatttaaaaatgtta

SEQ ID NO: 50 - DNA, 25 bp, Artificial Sequence
  Description of Artificial Sequence Synthetic oligonucleotide
agattaccatagatttaaaaatgtt

SEQ ID NO: 51 - DNA, 25 bp, Artificial Sequence
  Description of Artificial Sequence Synthetic oligonucleotide
aagattaccatagatttaaaaatgt

SEQ ID NO: 52 - DNA, 25 bp, Artificial Sequence
  Description of Artificial Sequence Synthetic oligonucleotide
aaagattaccatagatttaaaaatg

SEQ ID NO: 53 - DNA, 25 bp, Artificial Sequence
  Description of Artificial Sequence Synthetic oligonucleotide
taaagattaccatagatttaaaaat

SEQ ID NO: 54 - DNA, 25 bp, Artificial Sequence
  Description of Artificial Sequence Synthetic oligonucleotide
gtaaagattaccatagatttaaaaa

SEQ ID NO: 55 - DNA, 25 bp, Artificial Sequence
  Description of Artificial Sequence Synthetic oligonucleotide
tgtaaagattaccatagatttaaaa

SEQ ID NO: 56 - DNA, 25 bp, Artificial Sequence
  Description of Artificial Sequence Synthetic oligonucleotide
ttgtaaagattaccatagatttaaa

SEQ ID NO: 57 - DNA, 25 bp, Artificial Sequence
  Description of Artificial Sequence Synthetic oligonucleotide
tttgtaaagattaccatagatttaa

SEQ ID NO: 58 - DNA, 25 bp, Artificial Sequence
  Description of Artificial Sequence Synthetic oligonucleotide
ttttgtaaagattaccatagattta

SEQ ID NO: 59 - DNA, 25 bp, Artificial Sequence
  Description of Artificial Sequence Synthetic oligonucleotide
attttgtaaagattaccatagattt

SEQ ID NO: 60 - DNA, 25 bp, Artificial Sequence
  Description of Artificial Sequence Synthetic oligonucleotide
tattttgtaaagattaccatagatt

SEQ ID NO: 61 - DNA, 25 bp, Artificial Sequence
  Description of Artificial Sequence Synthetic oligonucleotide
atattttgtaaagattaccatagat

SEQ ID NO: 62 - DNA, 25 bp, Artificial Sequence
  Description of Artificial Sequence Synthetic oligonucleotide
aatattttgtaaagattaccataga

SEQ ID NO: 63 - DNA, 25 bp, Artificial Sequence
  Description of Artificial Sequence Synthetic oligonucleotide
aaatattttgtaaagattaccatag

SEQ ID NO: 64 - DNA, 25 bp, Artificial Sequence
  Description of Artificial Sequence Synthetic oligonucleotide
aaaatattttgtaaagattaccata

SEQ ID NO: 65 - DNA, 25 bp, Artificial Sequence
  Description of Artificial Sequence Synthetic oligonucleotide
taaaatattttgtaaagattaccat

SEQ ID NO: 66 - DNA, 25 bp, Artificial Sequence
  Description of Artificial Sequence Synthetic oligonucleotide
gtaaaatattttgtaaagattacca

SEQ ID NO: 67 - DNA, 25 bp, Artificial Sequence
  Description of Artificial Sequence Synthetic oligonucleotide
agtaaaatattttgtaaagattacc

SEQ ID NO: 68 - DNA, 25 bp, Artificial Sequence
  Description of Artificial Sequence Synthetic oligonucleotide
aagtaaaatattttgtaaagattac

SEQ ID NO: 69 - DNA, 25 bp, Artificial Sequence
  Description of Artificial Sequence Synthetic oligonucleotide
gaagtaaaatattttgtaaagatta

SEQ ID NO: 70 - DNA, 25 bp, Artificial Sequence
  Description of Artificial Sequence Synthetic oligonucleotide
ggaagtaaaatattttgtaaagatt

SEQ ID NO: 71 - DNA, 25 bp, Artificial Sequence
  Description of Artificial Sequence Synthetic oligonucleotide
cggaagtaaaatattttgtaaagat

SEQ ID NO: 72 - DNA, 25 bp, Artificial Sequence
  Description of Artificial Sequence Synthetic oligonucleotide
tcggaagtaaaatattttgtaaaga

SEQ ID NO: 73 - DNA, 25 bp, Artificial Sequence
  Description of Artificial Sequence Synthetic oligonucleotide
ttcggaagtaaaatattttgtaaag

SEQ ID NO: 74 - DNA, 25 bp, Artificial Sequence
  Description of Artificial Sequence Synthetic oligonucleotide
gttcggaagtaaaatattttgtaaa

SEQ ID NO: 75 - DNA, 25 bp, Artificial Sequence
  Description of Artificial Sequence Synthetic oligonucleotide
agttcggaagtaaaatattttgtaa

SEQ ID NO: 76 - DNA, 25 bp, Artificial Sequence
  Description of Artificial Sequence Synthetic oligonucleotide
gagttcggaagtaaaatattttgta

SEQ ID NO: 77 - DNA, 25 bp, Artificial Sequence
  Description of Artificial Sequence Synthetic oligonucleotide
tgagttcggaagtaaaatattttgt

SEQ ID NO: 78 - DNA, 25 bp, Artificial Sequence
  Description of Artificial Sequence Synthetic oligonucleotide
atgagttcggaagtaaaatattttg

SEQ ID NO: 79 - DNA, 25 bp, Artificial Sequence
  Description of Artificial Sequence Synthetic oligonucleotide
tatgagttcggaagtaaaatatttt

SEQ ID NO: 80 - DNA, 25 bp, Artificial Sequence
  Description of Artificial Sequence Synthetic oligonucleotide
atatgagttcggaagtaaaatattt

SEQ ID NO: 81 - DNA, 25 bp, Artificial Sequence
  Description of Artificial Sequence Synthetic oligonucleotide
tatatgagttcggaagtaaaatatt

SEQ ID NO: 82 - DNA, 25 bp, Artificial Sequence
  Description of Artificial Sequence Synthetic oligonucleotide
gtatatgagttcggaagtaaaatat

SEQ ID NO: 83 - DNA, 25 bp, Artificial Sequence
  Description of Artificial Sequence Synthetic oligonucleotide
ggtatatgagttcggaagtaaaata

SEQ ID NO: 84 - DNA, 25 bp, Artificial Sequence
  Description of Artificial Sequence Synthetic oligonucleotide
aggtatatgagttcggaagtaaaat

SEQ ID NO: 85 - DNA, 25 bp, Artificial Sequence
  Description of Artificial Sequence Synthetic oligonucleotide
caggtatatgagttcggaagtaaaa

SEQ ID NO: 86 - DNA, 25 bp, Artificial Sequence
  Description of Artificial Sequence Synthetic oligonucleotide
ccaggtatatgagttcggaagtaaa

SEQ ID NO: 87 - DNA, 25 bp, Artificial Sequence
  Description of Artificial Sequence Synthetic oligonucleotide
cccaggtatatgagttcggaagtaa

SEQ ID NO: 88 - DNA, 25 bp, Artificial Sequence
  Description of Artificial Sequence Synthetic oligonucleotide
ccccaggtatatgagttcggaagta

SEQ ID NO: 89 - DNA, 25 bp, Artificial Sequence
  Description of Artificial Sequence Synthetic oligonucleotide
tccccaggtatatgagttcggaagt

SEQ ID NO: 90 - DNA, 25 bp, Artificial Sequence
  Description of Artificial Sequence Synthetic oligonucleotide
atccccaggtatatgagttcggaag

SEQ ID NO: 91 - DNA, 25 bp, Artificial Sequence
  Description of Artificial Sequence Synthetic oligonucleotide
aatccccaggtatatgagttcggaa

SEQ ID NO: 92 - DNA, 25 bp, Artificial Sequence
  Description of Artificial Sequence Synthetic oligonucleotide
aaatccccaggtatatgagttcgga

SEQ ID NO: 93 - DNA, 25 bp, Artificial Sequence
  Description of Artificial Sequence Synthetic oligonucleotide
aaaatccccaggtatatgagttcgg

SEQ ID NO: 94 - DNA, 25 bp, Artificial Sequence
  Description of Artificial Sequence Synthetic oligonucleotide
taaaatccccaggtatatgagttcg

SEQ ID NO: 95 - DNA, 25 bp, Artificial Sequence
  Description of Artificial Sequence Synthetic oligonucleotide
ataaaatccccaggtatatgagttc

SEQ ID NO: 96 - DNA, 25 bp, Artificial Sequence
  Description of Artificial Sequence Synthetic oligonucleotide
aataaaatccccaggtatatgagtt

SEQ ID NO: 97 - DNA, 25 bp, Artificial Sequence
  Description of Artificial Sequence Synthetic oligonucleotide
taataaaatccccaggtatatgagt

SEQ ID NO: 98 - DNA, 25 bp, Artificial Sequence
  Description of Artificial Sequence Synthetic oligonucleotide
gtaataaaatccccaggtatatgag

SEQ ID NO: 99 - DNA, 25 bp, Artificial Sequence
  Description of Artificial Sequence Synthetic oligonucleotide
agtaataaaatccccaggtatatga

SEQ ID NO: 100 - DNA, 25 bp, Artificial Sequence
  Description of Artificial Sequence Synthetic oligonucleotide
gagtaataaaatccccaggtatatg

SEQ ID NO: 101 - DNA, 25 bp, Artificial Sequence
  Description of Artificial Sequence Synthetic oligonucleotide
agagtaataaaatccccaggtatat

SEQ ID NO: 102 - DNA, 25 bp, Artificial Sequence
  Description of Artificial Sequence Synthetic oligonucleotide
cagagtaataaaatccccaggtata

SEQ ID NO: 103 - DNA, 25 bp, Artificial Sequence
  Description of Artificial Sequence Synthetic oligonucleotide
ccagagtaataaaatccccaggtat

SEQ ID NO: 104 - DNA, 25 bp, Artificial Sequence
  Description of Artificial Sequence Synthetic oligonucleotide
cccagagtaataaaatccccaggta

SEQ ID NO: 105 - DNA, 25 bp, Artificial Sequence
  Description of Artificial Sequence Synthetic oligonucleotide
tcccagagtaataaaatccccaggt

SEQ ID NO: 106 - DNA, 25 bp, Artificial Sequence
  Description of Artificial Sequence Synthetic oligonucleotide
ttcccagagtaataaaatccccagg

SEQ ID NO: 107 - DNA, 25 bp, Artificial Sequence
  Description of Artificial Sequence Synthetic oligonucleotide
attcccagagtaataaaatccccag

SEQ ID NO: 108 - DNA, 25 bp, Artificial Sequence
  Description of Artificial Sequence Synthetic oligonucleotide
aattcccagagtaataaaatcccca

SEQ ID NO: 109 - DNA, 25 bp, Artificial Sequence
  Description of Artificial Sequence Synthetic oligonucleotide
taattcccagagtaataaaatcccc

SEQ ID NO: 110 - DNA, 25 bp, Artificial Sequence
  Description of Artificial Sequence Synthetic oligonucleotide
ataattcccagagtaataaaatccc

SEQ ID NO: 111 - DNA, 25 bp, Artificial Sequence
  Description of Artificial Sequence Synthetic oligonucleotide
cataattcccagagtaataaaatcc

SEQ ID NO: 112 - DNA, 25 bp, Artificial Sequence
  Description of Artificial Sequence Synthetic oligonucleotide
acataattcccagagtaataaaatc

SEQ ID NO: 113 - DNA, 25 bp, Artificial Sequence
  Description of Artificial Sequence Synthetic oligonucleotide
cacataattcccagagtaataaaat

SEQ ID NO: 114 - DNA, 25 bp, Artificial Sequence
  Description of Artificial Sequence Synthetic oligonucleotide
acacataattcccagagtaataaaa

SEQ ID NO: 115 - DNA, 25 bp, Artificial Sequence
  Description of Artificial Sequence Synthetic oligonucleotide
aacacataattcccagagtaataaa

SEQ ID NO: 116 - DNA, 25 bp, Artificial Sequence
  Description of Artificial Sequence Synthetic oligonucleotide
gaacacataattcccagagtaataa

SEQ ID NO: 117 - DNA, 25 bp, Artificial Sequence
  Description of Artificial Sequence Synthetic oligonucleotide
agaacacataattcccagagtaata

SEQ ID NO: 118 - DNA, 25 bp, Artificial Sequence
  Description of Artificial Sequence Synthetic oligonucleotide
cagaacacataattcccagagtaat

SEQ ID NO: 119 - DNA, 25 bp, Artificial Sequence
  Description of Artificial Sequence Synthetic oligonucleotide
gcagaacacataattcccagagtaa

SEQ ID NO: 120 - DNA, 25 bp, Artificial Sequence
  Description of Artificial Sequence Synthetic oligonucleotide
ggcagaacacataattcccagagta

SEQ ID NO: 121 - DNA, 25 bp, Artificial Sequence
  Description of Artificial Sequence Synthetic oligonucleotide
gggcagaacacataattcccagagt

SEQ ID NO: 122 - DNA, 25 bp, Artificial Sequence
  Description of Artificial Sequence Synthetic oligonucleotide
ggggcagaacacataattcccagag

SEQ ID NO: 123 - DNA, 25 bp, Artificial Sequence
  Description of Artificial Sequence Synthetic oligonucleotide
tggggcagaacacataattcccaga

SEQ ID NO: 124 - DNA, 25 bp, Artificial Sequence
  Description of Artificial Sequence Synthetic oligonucleotide
atggggcagaacacataattcccag

SEQ ID NO: 125 - DNA, 25 bp, Artificial Sequence
  Description of Artificial Sequence Synthetic oligonucleotide
gatggggcagaacacataattccca

SEQ ID NO: 126 - DNA, 25 bp, Artificial Sequence
  Description of Artificial Sequence Synthetic oligonucleotide
tgatggggcagaacacataattccc

SEQ ID NO: 127 - DNA, 25 bp, Artificial Sequence
  Description of Artificial Sequence Synthetic oligonucleotide
gtgatggggcagaacacataattcc

SEQ ID NO: 128 - DNA, 25 bp, Artificial Sequence
  Description of Artificial Sequence Synthetic oligonucleotide
agtgatggggcagaacacataattc

SEQ ID NO: 129 - DNA, 25 bp, Artificial Sequence
  Description of Artificial Sequence Synthetic oligonucleotide
gagtgatggggcagaacacataatt

SEQ ID NO: 130 - DNA, 25 bp, Artificial Sequence
  Description of Artificial Sequence Synthetic oligonucleotide
agagtgatggggcagaacacataat

SEQ ID NO: 131 - DNA, 25 bp, Artificial Sequence
  Description of Artificial Sequence Synthetic oligonucleotide
gagagtgatggggcagaacacataa

SEQ ID NO: 132 - DNA, 25 bp, Artificial Sequence
  Description of Artificial Sequence Synthetic oligonucleotide
agagagtgatggggcagaacacata

SEQ ID NO: 133 - DNA, 25 bp, Artificial Sequence
  Description of Artificial Sequence Synthetic oligonucleotide
gagagagtgatggggcagaacacat

SEQ ID NO: 134 - DNA, 25 bp, Artificial Sequence
  Description of Artificial Sequence Synthetic oligonucleotide
agagagagtgatggggcagaacaca

SEQ ID NO: 135 - DNA, 25 bp, Artificial Sequence
  Description of Artificial Sequence Synthetic oligonucleotide
aagagagagtgatggggcagaacac

SEQ ID NO: 136 - DNA, 25 bp, Artificial Sequence
  Description of Artificial Sequence Synthetic oligonucleotide
taagagagagtgatggggcagaaca

SEQ ID NO: 137 - DNA, 25 bp, Artificial Sequence
  Description of Artificial Sequence Synthetic oligonucleotide
ttaagagagagtgatggggcagaac

SEQ ID NO: 138 - DNA, 25 bp, Artificial Sequence
  Description of Artificial Sequence Synthetic oligonucleotide
attaagagagagtgatggggcagaa

SEQ ID NO: 139 - DNA, 25 bp, Artificial Sequence
  Description of Artificial Sequence Synthetic oligonucleotide
aattaagagagagtgatggggcaga

SEQ ID NO: 140 - DNA, 25 bp, Artificial Sequence
  Description of Artificial Sequence Synthetic oligonucleotide
caattaagagagagtgatggggcag

SEQ ID NO: 141 - DNA, 25 bp, Artificial Sequence
  Description of Artificial Sequence Synthetic oligonucleotide
ccaattaagagagagtgatggggca

SEQ ID NO: 142 - DNA, 25 bp, Artificial Sequence
  Description of Artificial Sequence Synthetic oligonucleotide
tccaattaagagagagtgatggggc

SEQ ID NO: 143 - DNA, 25 bp, Artificial Sequence
  Description of Artificial Sequence Synthetic oligonucleotide
atccaattaagagagagtgatgggg

SEQ ID NO: 144 - DNA, 25 bp, Artificial Sequence
  Description of Artificial Sequence Synthetic oligonucleotide
aatccaattaagagagagtgatggg

SEQ ID NO: 145 - DNA, 25 bp, Artificial Sequence
  Description of Artificial Sequence Synthetic oligonucleotide
aaatccaattaagagagagtgatgg

SEQ ID NO: 146 - DNA, 25 bp, Artificial Sequence
  Description of Artificial Sequence Synthetic oligonucleotide
aaaatccaattaagagagagtgatg

SEQ ID NO: 147 - DNA, 25 bp, Artificial Sequence
  Description of Artificial Sequence Synthetic oligonucleotide
aaaaatccaattaagagagagtgat

SEQ ID NO: 148 - DNA, 25 bp, Artificial Sequence
  Description of Artificial Sequence Synthetic oligonucleotide
taaaaatccaattaagagagagtga

SEQ ID NO: 149 - DNA, 25 bp, Artificial Sequence
  Description of Artificial Sequence Synthetic oligonucleotide
ttaaaaatccaattaagagagagtg

SEQ ID NO: 150 - DNA, 25 bp, Artificial Sequence
  Description of Artificial Sequence Synthetic oligonucleotide
tttaaaaatccaattaagagagagt

SEQ ID NO: 151 - DNA, 25 bp, Artificial Sequence
  Description of Artificial Sequence Synthetic oligonucleotide
ttttaaaaatccaattaagagagag

SEQ ID NO: 152 - DNA, 25 bp, Artificial Sequence
  Description of Artificial Sequence Synthetic oligonucleotide
attttaaaaatccaattaagagaga

SEQ ID NO: 153 - DNA, 25 bp, Artificial Sequence
  Description of Artificial Sequence Synthetic oligonucleotide
aattttaaaaatccaattaagagag

SEQ ID NO: 154 - DNA, 25 bp, Artificial Sequence
  Description of Artificial Sequence Synthetic oligonucleotide
taattttaaaaatccaattaagaga

SEQ ID NO: 155 - DNA, 25 bp, Artificial Sequence
  Description of Artificial Sequence Synthetic oligonucleotide
ataattttaaaaatccaattaagag

SEQ ID NO: 156 - DNA, 25 bp, Artificial Sequence
  Description of Artificial Sequence Synthetic oligonucleotide
tataattttaaaaatccaattaaga

SEQ ID NO: 157 - DNA, 25 bp, Artificial Sequence
  Description of Artificial Sequence Synthetic oligonucleotide
atataattttaaaaatccaattaag

SEQ ID NO: 158 - DNA, 25 bp, Artificial Sequence
  Description of Artificial Sequence Synthetic oligonucleotide
aatataattttaaaaatccaattaa

SEQ ID NO: 159 - DNA, 25 bp, Artificial Sequence
  Description of Artificial Sequence Synthetic oligonucleotide
gaatataattttaaaaatccaatta

SEQ ID NO: 160 - DNA, 25 bp, Artificial Sequence
  Description of Artificial Sequence Synthetic oligonucleotide
tgaatataattttaaaaatccaatt

SEQ ID NO: 161 - DNA, 25 bp, Artificial Sequence
  Description of Artificial Sequence Synthetic oligonucleotide
atgaatataattttaaaaatccaat

SEQ ID NO: 162 - DNA, 25 bp, Artificial Sequence
  Description of Artificial Sequence Synthetic oligonucleotide
tatgaatataattttaaaaatccaa

SEQ ID NO: 163 - DNA, 25 bp, Artificial Sequence
  Description of Artificial Sequence Synthetic oligonucleotide
atatgaatataattttaaaaatcca

SEQ ID NO: 164 - DNA, 25 bp, Artificial Sequence
  Description of Artificial Sequence Synthetic oligonucleotide
aatatgaatataattttaaaaatcc

SEQ ID NO: 165 - DNA, 25 bp, Artificial Sequence
  Description of Artificial Sequence Synthetic oligonucleotide
caatatgaatataattttaaaaatc

SEQ ID NO: 166 - DNA, 25 bp, Artificial Sequence
  Description of Artificial Sequence Synthetic oligonucleotide
gcaatatgaatataattttaaaaat

SEQ ID NO: 167 - DNA, 25 bp, Artificial Sequence
  Description of Artificial Sequence Synthetic oligonucleotide
tgcaatatgaatataattttaaaaa

SEQ ID NO: 168 - DNA, 25 bp, Artificial Sequence
  Description of Artificial Sequence Synthetic oligonucleotide
ctgcaatatgaatataattttaaaa

SEQ ID NO: 169 - DNA, 25 bp, Artificial Sequence
  Description of Artificial Sequence Synthetic oligonucleotide
cctgcaatatgaatataattttaaa

SEQ ID NO: 170 - DNA, 25 bp, Artificial Sequence
  Description of Artificial Sequence Synthetic oligonucleotide
tcctgcaatatgaatataattttaa

SEQ ID NO: 171 - DNA, 25 bp, Artificial Sequence
  Description of Artificial Sequence Synthetic oligonucleotide
gtcctgcaatatgaatataatttta

SEQ ID NO: 172 - DNA, 25 bp, Artificial Sequence
  Description of Artificial Sequence Synthetic oligonucleotide
agtcctgcaatatgaatataatttt

SEQ ID NO: 173 - DNA, 25 bp, Artificial Sequence
  Description of Artificial Sequence Synthetic oligonucleotide
gagtcctgcaatatgaatataattt

SEQ ID NO: 174 - DNA, 25 bp, Artificial Sequence
  Description of Artificial Sequence Synthetic oligonucleotide
cgagtcctgcaatatgaatataatt

SEQ ID NO: 175 - DNA, 25 bp, Artificial Sequence
  Description of Artificial Sequence Synthetic oligonucleotide
ccgagtcctgcaatatgaatataat

SEQ ID NO: 176 - DNA, 25 bp, Artificial Sequence
  Description of Artificial Sequence Synthetic oligonucleotide
gccgagtcctgcaatatgaatataa

SEQ ID NO: 177 - DNA, 25 bp, Artificial Sequence
  Description of Artificial Sequence Synthetic oligonucleotide
tgccgagtcctgcaatatgaatata

SEQ ID NO: 178 - DNA, 25 bp, Artificial Sequence
  Description of Artificial Sequence Synthetic oligonucleotide
ctgccgagtcctgcaatatgaatat

SEQ ID NO: 179 - DNA, 25 bp, Artificial Sequence
  Description of Artificial Sequence Synthetic oligonucleotide
tctgccgagtcctgcaatatgaata

SEQ ID NO: 180 - DNA, 25 bp, Artificial Sequence
  Description of Artificial Sequence Synthetic oligonucleotide
ttctgccgagtcctgcaatatgaat

SEQ ID NO: 181 - DNA, 25 bp, Artificial Sequence
  Description of Artificial Sequence Synthetic oligonucleotide
cttctgccgagtcctgcaatatgaa

SEQ ID NO: 182 - DNA, 25 bp, Artificial Sequence
  Description of Artificial Sequence Synthetic oligonucleotide
tcttctgccgagtcctgcaatatga

SEQ ID NO: 183 - DNA, 25 bp, Artificial Sequence
  Description of Artificial Sequence Synthetic oligonucleotide
gtcttctgccgagtcctgcaatatg

SEQ ID NO: 184 - DNA, 25 bp, Artificial Sequence
  Description of Artificial Sequence Synthetic oligonucleotide
ggtcttctgccgagtcctgcaatat

SEQ ID NO: 185 - DNA, 25 bp, Artificial Sequence
  Description of Artificial Sequence Synthetic oligonucleotide
aggtcttctgccgagtcctgcaata

SEQ ID NO: 186 - DNA, 25 bp, Artificial Sequence
  Description of Artificial Sequence Synthetic oligonucleotide
aaggtcttctgccgagtcctgcaat

SEQ ID NO: 187 - DNA, 25 bp, Artificial Sequence
  Description of Artificial Sequence Synthetic oligonucleotide
gaaggtcttctgccgagtcctgcaa

SEQ ID NO: 188 - DNA, 25 bp, Artificial Sequence
  Description of Artificial Sequence Synthetic oligonucleotide
cgaaggtcttctgccgagtcctgca

SEQ ID NO: 189 - DNA, 25 bp, Artificial Sequence
  Description of Artificial Sequence Synthetic oligonucleotide
tcgaaggtcttctgccgagtcctgc

SEQ ID NO: 190 - DNA, 25 bp, Artificial Sequence
  Description of Artificial Sequence Synthetic oligonucleotide
ctcgaaggtcttctgccgagtcctg

SEQ ID NO: 191 - DNA, 25 bp, Artificial Sequence
  Description of Artificial Sequence Synthetic oligonucleotide
tctcgaaggtcttctgccgagtcct

SEQ ID NO: 192 - DNA, 25 bp, Artificial Sequence
  Description of Artificial Sequence Synthetic oligonucleotide
ctctcgaaggtcttctgccgagtcc

SEQ ID NO: 193 - DNA, 25 bp, Artificial Sequence
  Description of Artificial Sequence Synthetic oligonucleotide
tctctcgaaggtcttctgccgagtc

SEQ ID NO: 194 - DNA, 25 bp, Artificial Sequence
  Description of Artificial Sequence Synthetic oligonucleotide
ttctctcgaaggtcttctgccgagt

SEQ ID NO: 195 - DNA, 25 bp, Artificial Sequence
  Description of Artificial Sequence Synthetic oligonucleotide
tttctctcgaaggtcttctgccgag

SEQ ID NO: 196 - DNA, 25 bp, Artificial Sequence
  Description of Artificial Sequence Synthetic oligonucleotide
ctttctctcgaaggtcttctgccga

SEQ ID NO: 197 - DNA, 25 bp, Artificial Sequence
  Description of Artificial Sequence Synthetic oligonucleotide
cctttctctcgaaggtcttctgccg

SEQ ID NO: 198 - DNA, 25 bp, Artificial Sequence
  Description of Artificial Sequence Synthetic oligonucleotide
acctttctctcgaaggtcttctgcc

SEQ ID NO: 199 - DNA, 25 bp, Artificial Sequence
  Description of Artificial Sequence Synthetic oligonucleotide
tacctttctctcgaaggtcttctgc

SEQ ID NO: 200 - DNA, 25 bp, Artificial Sequence
  Description of Artificial Sequence Synthetic oligonucleotide
ctacctttctctcgaaggtcttctg

SEQ ID NO: 201 - DNA, 25 bp, Artificial Sequence
  Description of Artificial Sequence Synthetic oligonucleotide
tctacctttctctcgaaggtcttct

SEQ ID NO: 202 - DNA, 25 bp, Artificial Sequence
  Description of Artificial Sequence Synthetic oligonucleotide
ttctacctttctctcgaaggtcttc

SEQ ID NO: 203 - DNA, 25 bp, Artificial Sequence
  Description of Artificial Sequence Synthetic oligonucleotide
tttctacctttctctcgaaggtctt

SEQ ID NO: 204 - DNA, 25 bp, Artificial Sequence
  Description of Artificial Sequence Synthetic oligonucleotide
ttttctacctttctctcgaaggtct

SEQ ID NO: 205 - DNA, 25 bp, Artificial Sequence
  Description of Artificial Sequence Synthetic oligonucleotide
attttctacctttctctcgaaggtc

SEQ ID NO: 206 - DNA, 25 bp, Artificial Sequence
  Description of Artificial Sequence Synthetic oligonucleotide
tattttctacctttctctcgaaggt

SEQ ID NO: 207 - DNA, 25 bp, Artificial Sequence
  Description of Artificial Sequence Synthetic oligonucleotide
ttattttctacctttctctcgaagg

SEQ ID NO: 208 - DNA, 25 bp, Artificial Sequence
  Description of Artificial Sequence Synthetic oligonucleotide
cttattttctacctttctctcgaag

SEQ ID NO: 209 - DNA, 25 bp, Artificial Sequence
  Description of Artificial Sequence Synthetic oligonucleotide
tcttattttctacctttctctcgaa

SEQ ID NO: 210 - DNA, 25 bp, Artificial Sequence
  Description of Artificial Sequence Synthetic oligonucleotide
ttcttattttctacctttctctcga

SEQ ID NO: 211 - DNA, 25 bp, Artificial Sequence
  Description of Artificial Sequence Synthetic oligonucleotide
attcttattttctacctttctctcg

SEQ ID NO: 212 - DNA, 25 bp, Artificial Sequence
  Description of Artificial Sequence Synthetic oligonucleotide
aattcttattttctacctttctctc

SEQ ID NO: 213 - DNA, 25 bp, Artificial Sequence
  Description of Artificial Sequence Synthetic oligonucleotide
aaattcttattttctacctttctct

SEQ ID NO: 214 - DNA, 25 bp, Artificial Sequence
  Description of Artificial Sequence Synthetic oligonucleotide
caaattcttattttctacctttctc

SEQ ID NO: 215 - DNA, 25 bp, Artificial Sequence
  Description of Artificial Sequence Synthetic oligonucleotide
ccaaattcttattttctacctttct

SEQ ID NO: 216 - DNA, 25 bp, Artificial Sequence
  Description of Artificial Sequence Synthetic oligonucleotide
gccaaattcttattttctacctttc

SEQ ID NO: 217 - DNA, 25 bp, Artificial Sequence
  Description of Artificial Sequence Synthetic oligonucleotide
agccaaattcttattttctaccttt

SEQ ID NO: 218 - DNA, 25 bp, Artificial Sequence
  Description of Artificial Sequence Synthetic oligonucleotide
gagccaaattcttattttctacctt

SEQ ID NO: 219 - DNA, 25 bp, Artificial Sequence
  Description of Artificial Sequence Synthetic oligonucleotide
agagccaaattcttattttctacct

SEQ ID NO: 220 - DNA, 25 bp, Artificial Sequence
  Description of Artificial Sequence Synthetic oligonucleotide
gagagccaaattcttattttctacc

SEQ ID NO: 221 - DNA, 25 bp, Artificial Sequence
  Description of Artificial Sequence Synthetic oligonucleotide
agagagccaaattcttattttctac

SEQ ID NO: 222 - DNA, 25 bp, Artificial Sequence
  Description of Artificial Sequence Synthetic oligonucleotide
cagagagccaaattcttattttcta

SEQ ID NO: 223 - DNA, 25 bp, Artificial Sequence
  Description of Artificial Sequence Synthetic oligonucleotide
acagagagccaaattcttattttct

SEQ ID NO: 224 - DNA, 25 bp, Artificial Sequence
  Description of Artificial Sequence Synthetic oligonucleotide
cacagagagccaaattcttattttc

SEQ ID NO: 225 - DNA, 25 bp, Artificial Sequence
  Description of Artificial Sequence Synthetic oligonucleotide
acacagagagccaaattcttatttt

SEQ ID NO: 226 - DNA, 25 bp, Artificial Sequence
  Description of Artificial Sequence Synthetic oligonucleotide
cacacagagagccaaattcttattt

SEQ ID NO: 227 - DNA, 25 bp, Artificial Sequence
  Description of Artificial Sequence Synthetic oligonucleotide
tcacacagagagccaaattcttatt

SEQ ID NO: 228 - DNA, 25 bp, Artificial Sequence
  Description of Artificial Sequence Synthetic oligonucleotide
ctcacacagagagccaaattcttat

SEQ ID NO: 229 - DNA, 25 bp, Artificial Sequence
  Description of Artificial Sequence Synthetic oligonucleotide
gctcacacagagagccaaattctta

SEQ ID NO: 230 - DNA, 25 bp, Artificial Sequence
  Description of Artificial Sequence Synthetic oligonucleotide
tgctcacacagagagccaaattctt

SEQ ID NO: 231 - DNA, 25 bp, Artificial Sequence
  Description of Artificial Sequence Synthetic oligonucleotide
atgctcacacagagagccaaattct

SEQ ID NO: 232 - DNA, 25 bp, Artificial Sequence
  Description of Artificial Sequence Synthetic oligonucleotide
catgctcacacagagagccaaattc

SEQ ID NO: 233 - DNA, 25 bp, Artificial Sequence
  Description of Artificial Sequence Synthetic oligonucleotide
acatgctcacacagagagccaaatt

SEQ ID NO: 234 - DNA, 25 bp, Artificial Sequence
  Description of Artificial Sequence Synthetic oligonucleotide
cacatgctcacacagagagccaaat

SEQ ID NO: 235 - DNA, 25 bp, Artificial Sequence
  Description of Artificial Sequence Synthetic oligonucleotide
acacatgctcacacagagagccaaa

SEQ ID NO: 236 - DNA, 25 bp, Artificial Sequence
  Description of Artificial Sequence Synthetic oligonucleotide
cacacatgctcacacagagagccaa

SEQ ID NO: 237 - DNA, 25 bp, Artificial Sequence
  Description of Artificial Sequence Synthetic oligonucleotide
gcacacatgctcacacagagagcca

SEQ ID NO: 238 - DNA, 25 bp, Artificial Sequence
  Description of Artificial Sequence Synthetic oligonucleotide
cgcacacatgctcacacagagagcc

SEQ ID NO: 239 - DNA, 25 bp, Artificial Sequence
  Description of Artificial Sequence Synthetic oligonucleotide
acgcacacatgctcacacagagagc

SEQ ID NO: 240 - DNA, 25 bp, Artificial Sequence
  Description of Artificial Sequence Synthetic oligonucleotide
cacgcacacatgctcacacagagag

SEQ ID NO: 241 - DNA, 25 bp, Artificial Sequence
  Description of Artificial Sequence Synthetic oligonucleotide
acacgcacacatgctcacacagaga

SEQ ID NO: 242 - DNA, 25 bp, Artificial Sequence
  Description of Artificial Sequence Synthetic oligonucleotide
cacacgcacacatgctcacacagag

SEQ ID NO: 243 - DNA, 25 bp, Artificial Sequence
  Description of Artificial Sequence Synthetic oligonucleotide
acacacgcacacatgctcacacaga

SEQ ID NO: 244 - DNA, 25 bp, Artificial Sequence
  Description of Artificial Sequence Synthetic oligonucleotide
cacacacgcacacatgctcacacag

SEQ ID NO: 245 - DNA, 25 bp, Artificial Sequence
  Description of Artificial Sequence Synthetic oligonucleotide
gcacacacgcacacatgctcacaca

SEQ ID NO: 246 - DNA, 25 bp, Artificial Sequence
  Description of Artificial Sequence Synthetic oligonucleotide
cgcacacacgcacacatgctcacac

SEQ ID NO: 247 - DNA, 25 bp, Artificial Sequence
  Description of Artificial Sequence Synthetic oligonucleotide
tcgcacacacgcacacatgctcaca

SEQ ID NO: 248 - DNA, 25 bp, Artificial Sequence
  Description of Artificial Sequence Synthetic oligonucleotide
ctcgcacacacgcacacatgctcac

SEQ ID NO: 249 - DNA, 25 bp, Artificial Sequence
  Description of Artificial Sequence Synthetic oligonucleotide
tctcgcacacacgcacacatgctca

SEQ ID NO: 250 - DNA, 25 bp, Artificial Sequence
  Description of Artificial Sequence Synthetic oligonucleotide
ctctcgcacacacgcacacatgctc

SEQ ID NO: 251 - DNA, 25 bp, Artificial Sequence
  Description of Artificial Sequence Synthetic oligonucleotide
tctctcgcacacacgcacacatgct

SEQ ID NO: 252 - DNA, 25 bp, Artificial Sequence
  Description of Artificial Sequence Synthetic oligonucleotide
ctctctcgcacacacgcacacatgc

SEQ ID NO: 253 - DNA, 25 bp, Artificial Sequence
  Description of Artificial Sequence Synthetic oligonucleotide
tctctctcgcacacacgcacacatg

SEQ ID NO: 254 - DNA, 25 bp, Artificial Sequence
  Description of Artificial Sequence Synthetic oligonucleotide
ctctctctcgcacacacgcacacat

SEQ ID NO: 255 - DNA, 25 bp, Artificial Sequence
  Description of Artificial Sequence Synthetic oligonucleotide
tctctctctcgcacacacgcacaca

SEQ ID NO: 256 - DNA, 25 bp, Artificial Sequence
  Description of Artificial Sequence Synthetic oligonucleotide
ctctctctctcgcacacacgcacac

SEQ ID NO: 257 - DNA, 25 bp, Artificial Sequence
  Description of Artificial Sequence Synthetic oligonucleotide
tctctctctctcgcacacacgcaca

SEQ ID NO: 258 - DNA, 25 bp, Artificial Sequence
  Description of Artificial Sequence Synthetic oligonucleotide
gtctctctctctcgcacacacgcac

SEQ ID NO: 259 - DNA, 25 bp, Artificial Sequence
  Description of Artificial Sequence Synthetic oligonucleotide
tgtctctctctctcgcacacacgca

SEQ ID NO: 260 - DNA, 25 bp, Artificial Sequence
  Description of Artificial Sequence Synthetic oligonucleotide
ctgtctctctctctcgcacacacgc

SEQ ID NO: 261 - DNA, 25 bp, Artificial Sequence
  Description of Artificial Sequence Synthetic oligonucleotide
tctgtctctctctctcgcacacacg

SEQ ID NO: 262 - DNA, 25 bp, Artificial Sequence
  Description of Artificial Sequence Synthetic oligonucleotide
gtctgtctctctctctcgcacacac

SEQ ID NO: 263 - DNA, 25 bp, Artificial Sequence
  Description of Artificial Sequence Synthetic oligonucleotide
tgtctgtctctctctctcgcacaca

SEQ ID NO: 264 - DNA, 25 bp, Artificial Sequence
  Description of Artificial Sequence Synthetic oligonucleotide
ctgtctgtctctctctctcgcacac

SEQ ID NO: 265 - DNA, 25 bp, Artificial Sequence
  Description of Artificial Sequence Synthetic oligonucleotide
gctgtctgtctctctctctcgcaca

SEQ ID NO: 266 - DNA, 25 bp, Artificial Sequence
  Description of Artificial Sequence Synthetic oligonucleotide
ggctgtctgtctctctctctcgcac

SEQ ID NO: 267 - DNA, 25 bp, Artificial Sequence
  Description of Artificial Sequence Synthetic oligonucleotide
aggctgtctgtctctctctctcgca

SEQ ID NO: 268 - DNA, 25 bp, Artificial Sequence
  Description of Artificial Sequence Synthetic oligonucleotide
caggctgtctgtctctctctctcgc

SEQ ID NO: 269 - DNA, 25 bp, Artificial Sequence
  Description of Artificial Sequence Synthetic oligonucleotide
gcaggctgtctgtctctctctctcg

SEQ ID NO: 270 - DNA, 25 bp, Artificial Sequence
  Description of Artificial Sequence Synthetic oligonucleotide
ggcaggctgtctgtctctctctctc

SEQ ID NO: 271 - DNA, 25 bp, Artificial Sequence
  Description of Artificial Sequence Synthetic oligonucleotide
aggcaggctgtctgtctctctctct

SEQ ID NO: 272 - DNA, 25 bp, Artificial Sequence
  Description of Artificial Sequence Synthetic oligonucleotide
taggcaggctgtctgtctctctctc

SEQ ID NO: 273 - DNA, 25 bp, Artificial Sequence
  Description of Artificial Sequence Synthetic oligonucleotide
ttaggcaggctgtctgtctctctct

SEQ ID NO: 274 - DNA, 25 bp, Artificial Sequence
  Description of Artificial Sequence Synthetic oligonucleotide
cttaggcaggctgtctgtctctctc

SEQ ID NO: 275 - DNA, 25 bp, Artificial Sequence
  Description of Artificial Sequence Synthetic oligonucleotide
tcttaggcaggctgtctgtctctct

SEQ ID NO: 276 - DNA, 25 bp, Artificial Sequence
  Description of Artificial Sequence Synthetic oligonucleotide
ttcttaggcaggctgtctgtctctc

SEQ ID NO: 277 - DNA, 25 bp, Artificial Sequence
  Description of Artificial Sequence Synthetic oligonucleotide
cttcttaggcaggctgtctgtctct

SEQ ID NO: 278 - DNA, 25 bp, Artificial Sequence
  Description of Artificial Sequence Synthetic oligonucleotide
tcttcttaggcaggctgtctgtctc

SEQ ID NO: 279 - DNA, 25 bp, Artificial Sequence
  Description of Artificial Sequence Synthetic oligonucleotide
ttcttcttaggcaggctgtctgtct

SEQ ID NO: 280 - DNA, 25 bp, Artificial Sequence
  Description of Artificial Sequence Synthetic oligonucleotide
tttcttcttaggcaggctgtctgtc

SEQ ID NO: 281 - DNA, 25 bp, Artificial Sequence
  Description of Artificial Sequence Synthetic oligonucleotide
atttcttcttaggcaggctgtctgt

SEQ ID NO: 282 - DNA, 25 bp, Artificial Sequence
  Description of Artificial Sequence Synthetic oligonucleotide
catttcttcttaggcaggctgtctg

SEQ ID NO: 283 - DNA, 25 bp, Artificial Sequence
  Description of Artificial Sequence Synthetic oligonucleotide
tcatttcttcttaggcaggctgtct

SEQ ID NO: 284 - DNA, 25 bp, Artificial Sequence
  Description of Artificial Sequence Synthetic oligonucleotide
ttcatttcttcttaggcaggctgtc

SEQ ID NO: 285 - DNA, 25 bp, Artificial Sequence
  Description of Artificial Sequence Synthetic oligonucleotide
attcatttcttcttaggcaggctgt

SEQ ID NO: 286 - DNA, 25 bp, Artificial Sequence
  Description of Artificial Sequence Synthetic oligonucleotide
cattcatttcttcttaggcaggctg

SEQ ID NO: 287 - DNA, 25 bp, Artificial Sequence
  Description of Artificial Sequence Synthetic oligonucleotide
acattcatttcttcttaggcaggct

SEQ ID NO: 288 - DNA, 25 bp, Artificial Sequence
  Description of Artificial Sequence Synthetic oligonucleotide
cacattcatttcttcttaggcaggc

SEQ ID NO: 289 - DNA, 25 bp, Artificial Sequence
  Description of Artificial Sequence Synthetic oligonucleotide
tcacattcatttcttcttaggcagg

SEQ ID NO: 290 - DNA, 25 bp, Artificial Sequence
  Description of Artificial Sequence Synthetic oligonucleotide
ttcacattcatttcttcttaggcag

SEQ ID NO: 291 - DNA, 25 bp, Artificial Sequence
  Description of Artificial Sequence Synthetic oligonucleotide
attcacattcatttcttcttaggca

SEQ ID NO: 292 - DNA, 25 bp, Artificial Sequence
  Description of Artificial Sequence Synthetic oligonucleotide
cattcacattcatttcttcttaggc

SEQ ID NO: 293 - DNA, 25 bp, Artificial Sequence
  Description of Artificial Sequence Synthetic oligonucleotide
gcattcacattcatttcttcttagg

SEQ ID NO: 294 - DNA, 25 bp, Artificial Sequence
  Description of Artificial Sequence Synthetic oligonucleotide
cgcattcacattcatttcttcttag

SEQ ID NO: 295 - DNA, 25 bp, Artificial Sequence
  Description of Artificial Sequence Synthetic oligonucleotide
ccgcattcacattcatttcttctta

SEQ ID NO: 296 - DNA, 25 bp, Artificial Sequence
  Description of Artificial Sequence Synthetic oligonucleotide
gccgcattcacattcatttcttctt

SEQ ID NO: 297 - DNA, 25 bp, Artificial Sequence
  Description of Artificial Sequence Synthetic oligonucleotide
agccgcattcacattcatttcttct

SEQ ID NO: 298 - DNA, 25 bp, Artificial Sequence
  Description of Artificial Sequence Synthetic oligonucleotide
aagccgcattcacattcatttcttc

SEQ ID NO: 299 - DNA, 25 bp, Artificial Sequence
  Description of Artificial Sequence Synthetic oligonucleotide
caagccgcattcacattcatttctt

SEQ ID NO: 300 - DNA, 25 bp, Artificial Sequence
  Description of Artificial Sequence Synthetic oligonucleotide
acaagccgcattcacattcatttct

SEQ ID NO: 301 - DNA, 25 bp, Artificial Sequence
  Description of Artificial Sequence Synthetic oligonucleotide
cacaagccgcattcacattcatttc

SEQ ID NO: 302 - DNA, 25 bp, Artificial Sequence
  Description of Artificial Sequence Synthetic oligonucleotide
ccacaagccgcattcacattcattt

SEQ ID NO: 303 - DNA, 25 bp, Artificial Sequence
  Description of Artificial Sequence Synthetic oligonucleotide
gccacaagccgcattcacattcatt

SEQ ID NO: 304 - DNA, 25 bp, Artificial Sequence
  Description of Artificial Sequence Synthetic oligonucleotide
tgccacaagccgcattcacattcat

SEQ ID NO: 305 - DNA, 25 bp, Artificial Sequence
  Description of Artificial Sequence Synthetic oligonucleotide
gtgccacaagccgcattcacattca

SEQ ID NO: 306 - DNA, 25 bp, Artificial Sequence
  Description of Artificial Sequence Synthetic oligonucleotide
tgtgccacaagccgcattcacattc

SEQ ID NO: 307 - DNA, 25 bp, Artificial Sequence
  Description of Artificial Sequence Synthetic oligonucleotide
ctgtgccacaagccgcattcacatt

SEQ ID NO: 308 - DNA, 25 bp, Artificial Sequence
  Description of Artificial Sequence Synthetic oligonucleotide
actgtgccacaagccgcattcacat

SEQ ID NO: 309 - DNA, 25 bp, Artificial Sequence
  Description of Artificial Sequence Synthetic oligonucleotide
aactgtgccacaagccgcattcaca

SEQ ID NO: 310 - DNA, 25 bp, Artificial Sequence
  Description of Artificial Sequence Synthetic oligonucleotide
caactgtgccacaagccgcattcac

SEQ ID NO: 311 - DNA, 25 bp, Artificial Sequence
  Description of Artificial Sequence Synthetic oligonucleotide
tcaactgtgccacaagccgcattca

SEQ ID NO: 312 - DNA, 25 bp, Artificial Sequence
  Description of Artificial Sequence Synthetic oligonucleotide
gtcaactgtgccacaagccgcattc

SEQ ID NO: 313 - DNA, 25 bp, Artificial Sequence
  Description of Artificial Sequence Synthetic oligonucleotide
tgtcaactgtgccacaagccgcatt

SEQ ID NO: 314 - DNA, 25 bp, Artificial Sequence
  Description of Artificial Sequence Synthetic oligonucleotide
ttgtcaactgtgccacaagccgcat

SEQ ID NO: 315 - DNA, 25 bp, Artificial Sequence
  Description of Artificial Sequence Synthetic oligonucleotide
cttgtcaactgtgccacaagccgca

SEQ ID NO: 316 - DNA, 25 bp, Artificial Sequence
  Description of Artificial Sequence Synthetic oligonucleotide
ccttgtcaactgtgccacaagccgc

SEQ ID NO: 317 - DNA, 25 bp, Artificial Sequence
  Description of Artificial Sequence Synthetic oligonucleotide
tccttgtcaactgtgccacaagccg

SEQ ID NO: 318 - DNA, 25 bp, Artificial Sequence
  Description of Artificial Sequence Synthetic oligonucleotide
atccttgtcaactgtgccacaagcc

SEQ ID NO: 319 - DNA, 25 bp, Artificial Sequence
  Description of Artificial Sequence Synthetic oligonucleotide
catccttgtcaactgtgccacaagc

SEQ ID NO: 320 - DNA, 25 bp, Artificial Sequence
  Description of Artificial Sequence Synthetic oligonucleotide
tcatccttgtcaactgtgccacaag

SEQ ID NO: 321 - DNA, 25 bp, Artificial Sequence
  Description of Artificial Sequence Synthetic oligonucleotide
atcatccttgtcaactgtgccacaa

SEQ ID NO: 322 - DNA, 25 bp, Artificial Sequence
  Description of Artificial Sequence Synthetic oligonucleotide
tatcatccttgtcaactgtgccaca

SEQ ID NO: 323 - DNA, 25 bp, Artificial Sequence
  Description of Artificial Sequence Synthetic oligonucleotide
ttatcatccttgtcaactgtgccac

SEQ ID NO: 324 - DNA, 25 bp, Artificial Sequence
  Description of Artificial Sequence Synthetic oligonucleotide
tttatcatccttgtcaactgtgcca

SEQ ID NO: 325 - DNA, 25 bp, Artificial Sequence
  Description of Artificial Sequence Synthetic oligonucleotide
atttatcatccttgtcaactgtgcc

SEQ ID NO: 326 - DNA, 25 bp, Artificial Sequence
  Description of Artificial Sequence Synthetic oligonucleotide
gatttatcatccttgtcaactgtgc

SEQ ID NO: 327 - DNA, 25 bp, Artificial Sequence
  Description of Artificial Sequence Synthetic oligonucleotide
tgatttatcatccttgtcaactgtg

SEQ ID NO: 328 - DNA, 25 bp, Artificial Sequence
  Description of Artificial Sequence Synthetic oligonucleotide
ttgatttatcatccttgtcaactgt

SEQ ID NO: 329 - DNA, 25 bp, Artificial Sequence
  Description of Artificial Sequence Synthetic oligonucleotide
attgatttatcatccttgtcaactg

SEQ ID NO: 330 - DNA, 25 bp, Artificial Sequence
  Description of Artificial Sequence Synthetic oligonucleotide
tattgatttatcatccttgtcaact

SEQ ID NO: 331 - DNA, 25 bp, Artificial Sequence
  Description of Artificial Sequence Synthetic oligonucleotide
ttattgatttatcatccttgtcaac

SEQ ID NO: 332 - DNA, 25 bp, Artificial Sequence
  Description of Artificial Sequence Synthetic oligonucleotide
attattgatttatcatccttgtcaa

SEQ ID NO: 333 - DNA, 25 bp, Artificial Sequence
  Description of Artificial Sequence Synthetic oligonucleotide
cattattgatttatcatccttgtca

SEQ ID NO: 334 - DNA, 25 bp, Artificial Sequence
  Description of Artificial Sequence Synthetic oligonucleotide
gcattattgatttatcatccttgtc

SEQ ID NO: 335 - DNA, 25 bp, Artificial Sequence
  Description of Artificial Sequence Synthetic oligonucleotide
tgcattattgatttatcatccttgt

SEQ ID NO: 336 - DNA, 25 bp, Artificial Sequence
  Description of Artificial Sequence Synthetic oligonucleotide
ttgcattattgatttatcatccttg

SEQ ID NO: 337 - DNA, 25 bp, Artificial Sequence
  Description of Artificial Sequence Synthetic oligonucleotide
cttgcattattgatttatcatcctt

SEQ ID NO: 338 - DNA, 25 bp, Artificial Sequence
  Description of Artificial Sequence Synthetic oligonucleotide
gcttgcattattgatttatcatcct

SEQ ID NO: 339 - DNA, 25 bp, Artificial Sequence
  Description of Artificial Sequence Synthetic oligonucleotide
agcttgcattattgatttatcatcc

SEQ ID NO: 340 - DNA, 25 bp, Artificial Sequence
  Description of Artificial Sequence Synthetic oligonucleotide
aagcttgcattattgatttatcatc

SEQ ID NO: 341 - DNA, 25 bp, Artificial Sequence
  Description of Artificial Sequence Synthetic oligonucleotide
taagcttgcattattgatttatcat

SEQ ID NO: 342 - DNA, 25 bp, Artificial Sequence
  Description of Artificial Sequence Synthetic oligonucleotide
gtaagcttgcattattgatttatca

SEQ ID NO: 343 - DNA, 25 bp, Artificial Sequence
  Description of Artificial Sequence Synthetic oligonucleotide
agtaagcttgcattattgatttatc

SEQ ID NO: 344 - DNA, 25 bp, Artificial Sequence
  Description of Artificial Sequence Synthetic oligonucleotide
tagtaagcttgcattattgatttat

SEQ ID NO: 345 - DNA, 25 bp, Artificial Sequence
  Description of Artificial Sequence Synthetic oligonucleotide
atagtaagcttgcattattgattta

SEQ ID NO: 346 - DNA, 25 bp, Artificial Sequence
  Description of Artificial Sequence Synthetic oligonucleotide
gatagtaagcttgcattattgattt

SEQ ID NO: 347 - DNA, 25 bp, Artificial Sequence
  Description of Artificial Sequence Synthetic oligonucleotide
tgatagtaagcttgcattattgatt

SEQ ID NO: 348 - DNA, 25 bp, Artificial Sequence
  Description of Artificial Sequence Synthetic oligonucleotide
atgatagtaagcttgcattattgat

SEQ ID NO: 349 - DNA, 25 bp, Artificial Sequence
  Description of Artificial Sequence Synthetic oligonucleotide
aatgatagtaagcttgcattattga

SEQ ID NO: 350 - DNA, 25 bp, Artificial Sequence
  Description of Artificial Sequence Synthetic oligonucleotide
aaatgatagtaagcttgcattattg

SEQ ID NO: 351 - DNA, 25 bp, Artificial Sequence
  Description of Artificial Sequence Synthetic oligonucleotide
taaatgatagtaagcttgcattatt

SEQ ID NO: 352 - DNA, 25 bp, Artificial Sequence
  Description of Artificial Sequence Synthetic oligonucleotide
ataaatgatagtaagcttgcattat

SEQ ID NO: 353 - DNA, 25 bp, Artificial Sequence
  Description of Artificial Sequence Synthetic oligonucleotide
cataaatgatagtaagcttgcatta

SEQ ID NO: 354 - DNA, 25 bp, Artificial Sequence
  Description of Artificial Sequence Synthetic oligonucleotide
tcataaatgatagtaagcttgcatt

SEQ ID NO: 355 - DNA, 25 bp, Artificial Sequence
  Description of Artificial Sequence Synthetic oligonucleotide
ttcataaatgatagtaagcttgcat

SEQ ID NO: 356 - DNA, 25 bp, Artificial Sequence
  Description of Artificial Sequence Synthetic oligonucleotide
attcataaatgatagtaagcttgca

SEQ ID NO: 357 - DNA, 25 bp, Artificial Sequence
  Description of Artificial Sequence Synthetic oligonucleotide
tattcataaatgatagtaagcttgc

SEQ ID NO: 358 - DNA, 25 bp, Artificial Sequence
  Description of Artificial Sequence Synthetic oligonucleotide
ctattcataaatgatagtaagcttg

SEQ ID NO: 359 - DNA, 25 bp, Artificial Sequence
  Description of Artificial Sequence Synthetic oligonucleotide
gctattcataaatgatagtaagctt

SEQ ID NO: 360 - DNA, 25 bp, Artificial Sequence
  Description of Artificial Sequence Synthetic oligonucleotide
tgctattcataaatgatagtaagct

SEQ ID NO: 361 - DNA, 25 bp, Artificial Sequence
  Description of Artificial Sequence Synthetic oligonucleotide
ttgctattcataaatgatagtaagc

SEQ ID NO: 362 - DNA, 25 bp, Artificial Sequence
  Description of Artificial Sequence Synthetic oligonucleotide
attgctattcataaatgatagtaag

SEQ ID NO: 363 - DNA, 25 bp, Artificial Sequence
  Description of Artificial Sequence Synthetic oligonucleotide
tattgctattcataaatgatagtaa

SEQ ID NO: 364 - DNA, 25 bp, Artificial Sequence
  Description of Artificial Sequence Synthetic oligonucleotide
gtattgctattcataaatgatagta

SEQ ID NO: 365 - DNA, 25 bp, Artificial Sequence
  Description of Artificial Sequence Synthetic oligonucleotide
agtattgctattcataaatgatagt

SEQ ID NO: 366 - DNA, 25 bp, Artificial Sequence
  Description of Artificial Sequence Synthetic oligonucleotide
cagtattgctattcataaatgatag

SEQ ID NO: 367 - DNA, 25 bp, Artificial Sequence
  Description of Artificial Sequence Synthetic oligonucleotide
tcagtattgctattcataaatgata

SEQ ID NO: 368 - DNA, 25 bp, Artificial Sequence
  Description of Artificial Sequence Synthetic oligonucleotide
ttcagtattgctattcataaatgat

SEQ ID NO: 369 - DNA, 25 bp, Artificial Sequence
  Description of Artificial Sequence Synthetic oligonucleotide
cttcagtattgctattcataaatga

SEQ ID NO: 370 - DNA, 25 bp, Artificial Sequence
  Description of Artificial Sequence Synthetic oligonucleotide
tcttcagtattgctattcataaatg

SEQ ID NO: 371 - DNA, 25 bp, Artificial Sequence
  Description of Artificial Sequence Synthetic oligonucleotide
ttcttcagtattgctattcataaat

SEQ ID NO: 372 - DNA, 25 bp, Artificial Sequence
  Description of Artificial Sequence Synthetic oligonucleotide
tttcttcagtattgctattcataaa

SEQ ID NO: 373 - DNA, 25 bp, Artificial Sequence
  Description of Artificial Sequence Synthetic oligonucleotide
atttcttcagtattgctattcataa

SEQ ID NO: 374 - DNA, 25 bp, Artificial Sequence
  Description of Artificial Sequence Synthetic oligonucleotide
aatttcttcagtattgctattcata

SEQ ID NO: 375 - DNA, 25 bp, Artificial Sequence
  Description of Artificial Sequence Synthetic oligonucleotide
taatttcttcagtattgctattcat

SEQ ID NO: 376 - DNA, 25 bp, Artificial Sequence
  Description of Artificial Sequence Synthetic oligonucleotide
ttaatttcttcagtattgctattca

SEQ ID NO: 377 - DNA, 25 bp, Artificial Sequence
  Description of Artificial Sequence Synthetic oligonucleotide
tttaatttcttcagtattgctattc

SEQ ID NO: 378 - DNA, 25 bp, Artificial Sequence
  Description of Artificial Sequence Synthetic oligonucleotide
ttttaatttcttcagtattgctatt

SEQ ID NO: 379 - DNA, 25 bp, Artificial Sequence
  Description of Artificial Sequence Synthetic oligonucleotide
gttttaatttcttcagtattgctat

SEQ ID NO: 380 - DNA, 25 bp, Artificial Sequence
  Description of Artificial Sequence Synthetic oligonucleotide
tgttttaatttcttcagtattgcta

SEQ ID NO: 381 - DNA, 25 bp, Artificial Sequence
  Description of Artificial Sequence Synthetic oligonucleotide
ttgttttaatttcttcagtattgct

SEQ ID NO: 382 - DNA, 25 bp, Artificial Sequence
  Description of Artificial Sequence Synthetic oligonucleotide
tttgttttaatttcttcagtattgc

SEQ ID NO: 383 - DNA, 25 bp, Artificial Sequence
  Description of Artificial Sequence Synthetic oligonucleotide
ttttgttttaatttcttcagtattg

SEQ ID NO: 384 - DNA, 25 bp, Artificial Sequence
  Description of Artificial Sequence Synthetic oligonucleotide
cttttgttttaatttcttcagtatt

SEQ ID NO: 385 - DNA, 25 bp, Artificial Sequence
  Description of Artificial Sequence Synthetic oligonucleotide
tcttttgttttaatttcttcagtat

SEQ ID NO: 386 - DNA, 25 bp, Artificial Sequence
  Description of Artificial Sequence Synthetic oligonucleotide
atcttttgttttaatttcttcagta

SEQ ID NO: 387 - DNA, 25 bp, Artificial Sequence
  Description of Artificial Sequence Synthetic oligonucleotide
aatcttttgttttaatttcttcagt

SEQ ID NO: 388 - DNA, 25 bp, Artificial Sequence
  Description of Artificial Sequence Synthetic oligonucleotide
caatcttttgttttaatttcttcag

SEQ ID NO: 389 - DNA, 25 bp, Artificial Sequence
  Description of Artificial Sequence Synthetic oligonucleotide
gcaatcttttgttttaatttcttca

SEQ ID NO: 390 - DNA, 25 bp, Artificial Sequence
  Description of Artificial Sequence Synthetic oligonucleotide
agcaatcttttgttttaatttcttc

SEQ ID NO: 391 - DNA, 25 bp, Artificial Sequence
  Description of Artificial Sequence Synthetic oligonucleotide
cagcaatcttttgttttaatttctt

SEQ ID NO: 392 - DNA, 25 bp, Artificial Sequence
  Description of Artificial Sequence Synthetic oligonucleotide
acagcaatcttttgttttaatttct

SEQ ID NO: 393 - DNA, 25 bp, Artificial Sequence
  Description of Artificial Sequence Synthetic oligonucleotide
gacagcaatcttttgttttaatttc

SEQ ID NO: 394 - DNA, 25 bp, Artificial Sequence
  Description of Artificial Sequence Synthetic oligonucleotide
agacagcaatcttttgttttaattt

SEQ ID NO: 395 - DNA, 25 bp, Artificial Sequence
  Description of Artificial Sequence Synthetic oligonucleotide
gagacagcaatcttttgttttaatt

SEQ ID NO: 396 - DNA, 25 bp, Artificial Sequence
  Description of Artificial Sequence Synthetic oligonucleotide
tgagacagcaatcttttgttttaat

SEQ ID NO: 397 - DNA, 25 bp, Artificial Sequence
  Description of Artificial Sequence Synthetic oligonucleotide
ttgagacagcaatcttttgttttaa

SEQ ID NO: 398 - DNA, 25 bp, Artificial Sequence
  Description of Artificial Sequence Synthetic oligonucleotide
attgagacagcaatcttttgtttta

SEQ ID NO: 399 - DNA, 25 bp, Artificial Sequence
  Description of Artificial Sequence Synthetic oligonucleotide
tattgagacagcaatcttttgtttt

SEQ ID NO: 400 - DNA, 25 bp, Artificial Sequence
  Description of Artificial Sequence Synthetic oligonucleotide
atattgagacagcaatcttttgttt

SEQ ID NO: 401 - DNA, 25 bp, Artificial Sequence
  Description of Artificial Sequence Synthetic oligonucleotide
tatattgagacagcaatcttttgtt

SEQ ID NO: 402 - DNA, 25 bp, Artificial Sequence
  Description of Artificial Sequence Synthetic oligonucleotide
atatattgagacagcaatcttttgt

SEQ ID NO: 403 - DNA, 25 bp, Artificial Sequence
  Description of Artificial Sequence Synthetic oligonucleotide
gatatattgagacagcaatcttttg

SEQ ID NO: 404 - DNA, 25 bp, Artificial Sequence
  Description of Artificial Sequence Synthetic oligonucleotide
agatatattgagacagcaatctttt

SEQ ID NO: 405 - DNA, 25 bp, Artificial Sequence
  Description of Artificial Sequence Synthetic oligonucleotide
aagatatattgagacagcaatcttt

SEQ ID NO: 406 - DNA, 25 bp, Artificial Sequence
  Description of Artificial Sequence Synthetic oligonucleotide
taagatatattgagacagcaatctt

SEQ ID NO: 407 - DNA, 25 bp, Artificial Sequence
  Description of Artificial Sequence Synthetic oligonucleotide
ataagatatattgagacagcaatct

SEQ ID NO: 408 - DNA, 25 bp, Artificial Sequence
  Description of Artificial Sequence Synthetic oligonucleotide
tataagatatattgagacagcaatc

SEQ ID NO: 409 - DNA, 25 bp, Artificial Sequence
  Description of Artificial Sequence Synthetic oligonucleotide
atataagatatattgagacagcaat

SEQ ID NO: 410 - DNA, 25 bp, Artificial Sequence
  Description of Artificial Sequence Synthetic oligonucleotide
aatataagatatattgagacagcaa

SEQ ID NO: 411 - DNA, 25 bp, Artificial Sequence
  Description of Artificial Sequence Synthetic oligonucleotide
aaatataagatatattgagacagca

SEQ ID NO: 412 - DNA, 25 bp, Artificial Sequence
  Description of Artificial Sequence Synthetic oligonucleotide
taaatataagatatattgagacagc

SEQ ID NO: 413 - DNA, 25 bp, Artificial Sequence
  Description of Artificial Sequence Synthetic oligonucleotide
ataaatataagatatattgagacag

SEQ ID NO: 414 - DNA, 25 bp, Artificial Sequence
  Description of Artificial Sequence Synthetic oligonucleotide
aataaatataagatatattgagaca

SEQ ID NO: 415 - DNA, 25 bp, Artificial Sequence
  Description of Artificial Sequence Synthetic oligonucleotide
taataaatataagatatattgagac

SEQ ID NO: 416 - DNA, 25 bp, Artificial Sequence
  Description of Artificial Sequence Synthetic oligonucleotide
ataataaatataagatatattgaga

SEQ ID NO: 417 - DNA, 25 bp, Artificial Sequence
  Description of Artificial Sequence Synthetic oligonucleotide
aataataaatataagatatattgag

SEQ ID NO: 418 - DNA, 25 bp, Artificial Sequence
  Description of Artificial Sequence Synthetic oligonucleotide
aaataataaatataagatatattga

SEQ ID NO: 419 - DNA, 25 bp, Artificial Sequence
  Description of Artificial Sequence Synthetic oligonucleotide
taaataataaatataagatatattg

SEQ ID NO: 420 - DNA, 25 bp, Artificial Sequence
  Description of Artificial Sequence Synthetic oligonucleotide
gtaaataataaatataagatatatt

SEQ ID NO: 421 - DNA, 25 bp, Artificial Sequence
  Description of Artificial Sequence Synthetic oligonucleotide
ggtaaataataaatataagatatat

SEQ ID NO: 422 - DNA, 25 bp, Artificial Sequence
  Description of Artificial Sequence Synthetic oligonucleotide
tggtaaataataaatataagatata

SEQ ID NO: 423 - DNA, 25 bp, Artificial Sequence
  Description of Artificial Sequence Synthetic oligonucleotide
ttggtaaataataaatataagatat

SEQ ID NO: 424 - DNA, 25 bp, Artificial Sequence
  Description of Artificial Sequence Synthetic oligonucleotide
tttggtaaataataaatataagata

SEQ ID NO: 425 - DNA, 25 bp, Artificial Sequence
  Description of Artificial Sequence Synthetic oligonucleotide
atttggtaaataataaatataagat

SEQ ID NO: 426 - DNA, 25 bp, Artificial Sequence
  Description of Artificial Sequence Synthetic oligonucleotide
aatttggtaaataataaatataaga

SEQ ID NO: 427 - DNA, 25 bp, Artificial Sequence
  Description of Artificial Sequence Synthetic oligonucleotide
taatttggtaaataataaatataag

SEQ ID NO: 428 - DNA, 25 bp, Artificial Sequence
  Description of Artificial Sequence Synthetic oligonucleotide
ataatttggtaaataataaatataa

SEQ ID NO: 429 - DNA, 25 bp, Artificial Sequence
  Description of Artificial Sequence Synthetic oligonucleotide
aataatttggtaaataataaatata

SEQ ID NO: 430 - DNA, 25 bp, Artificial Sequence
  Description of Artificial Sequence Synthetic oligonucleotide
gaataatttggtaaataataaatat

SEQ ID NO: 431 - DNA, 25 bp, Artificial Sequence
  Description of Artificial Sequence Synthetic oligonucleotide
agaataatttggtaaataataaata

SEQ ID NO: 432 - DNA, 25 bp, Artificial Sequence
  Description of Artificial Sequence Synthetic oligonucleotide
tagaataatttggtaaataataaat

SEQ ID NO: 433 - DNA, 25 bp, Artificial Sequence
  Description of Artificial Sequence Synthetic oligonucleotide
ttagaataatttggtaaataataaa

SEQ ID NO: 434 - DNA, 25 bp, Artificial Sequence
  Description of Artificial Sequence Synthetic oligonucleotide
cttagaataatttggtaaataataa

SEQ ID NO: 435 - DNA, 25 bp, Artificial Sequence
  Description of Artificial Sequence Synthetic oligonucleotide
tcttagaataatttggtaaataata

SEQ ID NO: 436 - DNA, 25 bp, Artificial Sequence
  Description of Artificial Sequence Synthetic oligonucleotide
ctcttagaataatttggtaaataat

SEQ ID NO: 437 - DNA, 25 bp, Artificial Sequence
  Description of Artificial Sequence Synthetic oligonucleotide
actcttagaataatttggtaaataa

SEQ ID NO: 438 - DNA, 25 bp, Artificial Sequence
  Description of Artificial Sequence Synthetic oligonucleotide
tactcttagaataatttggtaaata

SEQ ID NO: 439 - DNA, 25 bp, Artificial Sequence
  Description of Artificial Sequence Synthetic oligonucleotide
atactcttagaataatttggtaaat

SEQ ID NO: 440 - DNA, 25 bp, Artificial Sequence
  Description of Artificial Sequence Synthetic oligonucleotide
aatactcttagaataatttggtaaa

SEQ ID NO: 441 - DNA, 25 bp, Artificial Sequence
  Description of Artificial Sequence Synthetic oligonucleotide
aaatactcttagaataatttggtaa

SEQ ID NO: 442 - DNA, 25 bp, Artificial Sequence
  Description of Artificial Sequence Synthetic oligonucleotide
gaaatactcttagaataatttggta

SEQ ID NO: 443 - DNA, 25 bp, Artificial Sequence
  Description of Artificial Sequence Synthetic oligonucleotide
agaaatactcttagaataatttggt

SEQ ID NO: 444 - DNA, 25 bp, Artificial Sequence
  Description of Artificial Sequence Synthetic oligonucleotide
aagaaatactcttagaataatttgg

SEQ ID NO: 445 - DNA, 25 bp, Artificial Sequence
  Description of Artificial Sequence Synthetic oligonucleotide
gaagaaatactcttagaataatttg

SEQ ID NO: 446 - DNA, 25 bp, Artificial Sequence
  Description of Artificial Sequence Synthetic oligonucleotide
ggaagaaatactcttagaataattt

SEQ ID NO: 447 - DNA, 227 bp, Homo sapiens
gactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctc

SEQ ID NO: 448 - DNA, 25 bp, Homo sapiens
acttgtaatatacaggtatccctcc

SEQ ID NO: 449 - DNA, 25 bp, Homo sapiens
cttgtaatatacaggtatccctcct

SEQ ID NO: 450 - DNA, 25 bp, Homo sapiens
ttgtaatatacaggtatccctcctg

SEQ ID NO: 451 - DNA, 25 bp, Homo sapiens
tgtaatatacaggtatccctcctgg

SEQ ID NO: 452 - DNA, 25 bp, Homo sapiens
gtaatatacaggtatccctcctggt

SEQ ID NO: 453 - DNA, 25 bp, Homo sapiens
taatatacaggtatccctcctggta

SEQ ID NO: 454 - DNA, 25 bp, Homo sapiens
aatatacaggtatccctcctggtaa

SEQ ID NO: 455 - DNA, 25 bp, Homo sapiens
atatacaggtatccctcctggtaag

SEQ ID NO: 456 - DNA, 25 bp, Homo sapiens
tatacaggtatccctcctggtaagc

SEQ ID NO: 457 - DNA, 25 bp, Homo sapiens
atacaggtatccctcctggtaagct

SEQ ID NO: 458 - DNA, 25 bp, Homo sapiens
tacaggtatccctcctggtaagctc

SEQ ID NO: 459 - DNA, 25 bp, Homo sapiens
acaggtatccctcctggtaagctct

SEQ ID NO: 460 - DNA, 25 bp, Homo sapiens
caggtatccctcctggtaagctctg

SEQ ID NO: 461 - DNA, 25 bp, Homo sapiens
aggtatccctcctggtaagctctgg

SEQ ID NO: 462 - DNA, 25 bp, Homo sapiens
ggtatccctcctggtaagctctggt

SEQ ID NO: 463 - DNA, 25 bp, Homo sapiens
gtatccctcctggtaagctctggta

SEQ ID NO: 464 - DNA, 25 bp, Homo sapiens
tatccctcctggtaagctctggtat

SEQ ID NO: 465 - DNA, 25 bp, Homo sapiens
atccctcctggtaagctctggtatt

SEQ ID NO: 466 - DNA, 25 bp, Homo sapiens
tccctcctggtaagctctggtatta

SEQ ID NO: 467 - DNA, 25 bp, Homo sapiens
ccctcctggtaagctctggtattat

SEQ ID NO: 468 - DNA, 25 bp, Homo sapiens
cctcctggtaagctctggtattatg

SEQ ID NO: 469 - DNA, 25 bp, Homo sapiens
ctcctggtaagctctggtattatgt

SEQ ID NO: 470 - DNA, 25 bp, Homo sapiens
tcctggtaagctctggtattatgtc

SEQ ID NO: 471 - DNA, 25 bp, Homo sapiens
cctggtaagctctggtattatgtct

SEQ ID NO: 472 - DNA, 25 bp, Homo sapiens
ctggtaagctctggtattatgtctt

SEQ ID NO: 473 - DNA, 25 bp, Homo sapiens
tggtaagctctggtattatgtctta

SEQ ID NO: 474 - DNA, 25 bp, Homo sapiens
ggtaagctctggtattatgtcttaa

SEQ ID NO: 475 - DNA, 25 bp, Homo sapiens
gtaagctctggtattatgtcttaac

SEQ ID NO: 476 - DNA, 25 bp, Homo sapiens
taagctctggtattatgtcttaaca

SEQ ID NO: 477 - DNA, 25 bp, Homo sapiens
aagctctggtattatgtcttaacat

SEQ ID NO: 478 - DNA, 25 bp, Homo sapiens
agctctggtattatgtcttaacatt

SEQ ID NO: 479 - DNA, 25 bp, Homo sapiens
gctctggtattatgtcttaacattt

SEQ ID NO: 480 - DNA, 25 bp, Homo sapiens
ctctggtattatgtcttaacatttt

SEQ ID NO: 481 - DNA, 25 bp, Homo sapiens
tctggtattatgtcttaacattttt

SEQ ID NO: 482 - DNA, 25 bp, Homo sapiens
ctggtattatgtcttaacattttta

SEQ ID NO: 483 - DNA, 25 bp, Homo sapiens
tggtattatgtcttaacatttttaa

SEQ ID NO: 484 - DNA, 25 bp, Homo sapiens
ggtattatgtcttaacatttttaaa

SEQ ID NO: 485 - DNA, 25 bp, Homo sapiens
gtattatgtcttaacatttttaaat

SEQ ID NO: 486 - DNA, 25 bp, Homo sapiens
tattatgtcttaacatttttaaatc

SEQ ID NO: 487 - DNA, 25 bp, Homo sapiens
attatgtcttaacatttttaaatct

SEQ ID NO: 488 - DNA, 25 bp, Homo sapiens
ttatgtcttaacatttttaaatcta

SEQ ID NO: 489 - DNA, 25 bp, Homo sapiens
tatgtcttaacatttttaaatctat

SEQ ID NO: 490 - DNA, 25 bp, Homo sapiens
atgtcttaacatttttaaatctatg

SEQ ID NO: 491 - DNA, 25 bp, Homo sapiens
tgtcttaacatttttaaatctatgg

SEQ ID NO: 492 - DNA, 25 bp, Homo sapiens
gtcttaacatttttaaatctatggt

SEQ ID NO: 493 - DNA, 25 bp, Homo sapiens
tcttaacatttttaaatctatggta

SEQ ID NO: 494 - DNA, 25 bp, Homo sapiens
cttaacatttttaaatctatggtaa

SEQ ID NO: 495 - DNA, 25 bp, Homo sapiens
ttaacatttttaaatctatggtaat

SEQ ID NO: 496 - DNA, 25 bp, Homo sapiens
taacatttttaaatctatggtaatc

SEQ ID NO: 497 - DNA, 25 bp, Homo sapiens
aacatttttaaatctatggtaatct

SEQ ID NO: 498 - DNA, 25 bp, Homo sapiens
acatttttaaatctatggtaatctt

SEQ ID NO: 499 - DNA, 25 bp, Homo sapiens
catttttaaatctatggtaatcttt

SEQ ID NO: 500 - DNA, 25 bp, Homo sapiens
atttttaaatctatggtaatcttta

SEQ ID NO: 501 - DNA, 25 bp, Homo sapiens
tttttaaatctatggtaatctttac

SEQ ID NO: 502 - DNA, 25 bp, Homo sapiens
ttttaaatctatggtaatctttaca

SEQ ID NO: 503 - DNA, 25 bp, Homo sapiens
tttaaatctatggtaatctttacaa

SEQ ID NO: 504 - DNA, 25 bp, Homo sapiens
ttaaatctatggtaatctttacaaa

SEQ ID NO: 505 - DNA, 25 bp, Homo sapiens
taaatctatggtaatctttacaaaa

SEQ ID NO: 506 - DNA, 25 bp, Homo sapiens
aaatctatggtaatctttacaaaat

SEQ ID NO: 507 - DNA, 25 bp, Homo sapiens
aatctatggtaatctttacaaaata

SEQ ID NO: 508 - DNA, 25 bp, Homo sapiens
atctatggtaatctttacaaaatat

SEQ ID NO: 509 - DNA, 25 bp, Homo sapiens
tctatggtaatctttacaaaatatt

SEQ ID NO: 510 - DNA, 25 bp, Homo sapiens
ctatggtaatctttacaaaatattt

SEQ ID NO: 511 - DNA, 25 bp, Homo sapiens
tatggtaatctttacaaaatatttt

SEQ ID NO: 512 - DNA, 25 bp, Homo sapiens
atggtaatctttacaaaatatttta

SEQ ID NO: 513 - DNA, 25 bp, Homo sapiens
tggtaatctttacaaaatattttac

SEQ ID NO: 514 - DNA, 25 bp, Homo sapiens
ggtaatctttacaaaatattttact

SEQ ID NO: 515 - DNA, 25 bp, Homo sapiens
gtaatctttacaaaatattttactt

SEQ ID NO: 516 - DNA, 25 bp, Homo sapiens
taatctttacaaaatattttacttc

SEQ ID NO: 517 - DNA, 25 bp, Homo sapiens
aatctttacaaaatattttacttcc

SEQ ID NO: 518 - DNA, 25 bp, Homo sapiens
atctttacaaaatattttacttccg

SEQ ID NO: 519 - DNA, 25 bp, Homo sapiens
tctttacaaaatattttacttccga

SEQ ID NO: 520 - DNA, 25 bp, Homo sapiens
ctttacaaaatattttacttccgaa

SEQ ID NO: 521 - DNA, 25 bp, Homo sapiens
tttacaaaatattttacttccgaac

SEQ ID NO: 522 - DNA, 25 bp, Homo sapiens
ttacaaaatattttacttccgaact

SEQ ID NO: 523 - DNA, 25 bp, Homo sapiens
tacaaaatattttacttccgaactc

SEQ ID NO: 524 - DNA, 25 bp, Homo sapiens
acaaaatattttacttccgaactca

SEQ ID NO: 525 - DNA, 25 bp, Homo sapiens
caaaatattttacttccgaactcat

SEQ ID NO: 526 - DNA, 25 bp, Homo sapiens
aaaatattttacttccgaactcata

SEQ ID NO: 527 - DNA, 25 bp, Homo sapiens
aaatattttacttccgaactcatat

SEQ ID NO: 528 - DNA, 25 bp, Homo sapiens
aatattttacttccgaactcatata

SEQ ID NO: 529 - DNA, 25 bp, Homo sapiens
atattttacttccgaactcatatac

SEQ ID NO: 530 - DNA, 25 bp, Homo sapiens
tattttacttccgaactcatatacc

SEQ ID NO: 531 - DNA, 25 bp, Homo sapiens
attttacttccgaactcatatacct

SEQ ID NO: 532 - DNA, 25 bp, Homo sapiens
ttttacttccgaactcatatacctg

SEQ ID NO: 533 - DNA, 25 bp, Homo sapiens
tttacttccgaactcatatacctgg

SEQ ID NO: 534 - DNA, 25 bp, Homo sapiens
ttacttccgaactcatatacctggg

SEQ ID NO: 535 - DNA, 25 bp, Homo sapiens
tacttccgaactcatatacctgggg

SEQ ID NO: 536 - DNA, 25 bp, Homo sapiens
acttccgaactcatatacctgggga

SEQ ID NO: 537 - DNA, 25 bp, Homo sapiens
cttccgaactcatatacctggggat

SEQ ID NO: 538 - DNA, 25 bp, Homo sapiens
ttccgaactcatatacctggggatt

SEQ ID NO: 539 - DNA, 25 bp, Homo sapiens
tccgaactcatatacctggggattt

SEQ ID NO: 540 - DNA, 25 bp, Homo sapiens
ccgaactcatatacctggggatttt

SEQ ID NO: 541 - DNA, 25 bp, Homo sapiens
cgaactcatatacctggggatttta

SEQ ID NO: 542 - DNA, 25 bp, Homo sapiens
gaactcatatacctggggattttat

SEQ ID NO: 543 - DNA, 25 bp, Homo sapiens
aactcatatacctggggattttatt

SEQ ID NO: 544 - DNA, 25 bp, Homo sapiens
actcatatacctggggattttatta

SEQ ID NO: 545 - DNA, 25 bp, Homo sapiens
ctcatatacctggggattttattac

SEQ ID NO: 546 - DNA, 25 bp, Homo sapiens
tcatatacctggggattttattact

SEQ ID NO: 547 - DNA, 25 bp, Homo sapiens
catatacctggggattttattactc

SEQ ID NO: 548 - DNA, 25 bp, Homo sapiens
atatacctggggattttattactct

SEQ ID NO: 549 - DNA, 25 bp, Homo sapiens
tatacctggggattttattactctg

SEQ ID NO: 550 - DNA, 25 bp, Homo sapiens
atacctggggattttattactctgg

SEQ ID NO: 551 - DNA, 25 bp, Homo sapiens
tacctggggattttattactctggg

SEQ ID NO: 552 - DNA, 25 bp, Homo sapiens
acctggggattttattactctggga

SEQ ID NO: 553 - DNA, 25 bp, Homo sapiens
cctggggattttattactctgggaa

SEQ ID NO: 554 - DNA, 25 bp, Homo sapiens
ctggggattttattactctgggaat

SEQ ID NO: 555 - DNA, 25 bp, Homo sapiens
tggggattttattactctgggaatt

SEQ ID NO: 556 - DNA, 25 bp, Homo sapiens
ggggattttattactctgggaatta

SEQ ID NO: 557 - DNA, 25 bp, Homo sapiens
gggattttattactctgggaattat

SEQ ID NO: 558 - DNA, 25 bp, Homo sapiens
ggattttattactctgggaattatg

SEQ ID NO: 559 - DNA, 25 bp, Homo sapiens
gattttattactctgggaattatgt

SEQ ID NO: 560 - DNA, 25 bp, Homo sapiens
attttattactctgggaattatgtg

SEQ ID NO: 561 - DNA, 25 bp, Homo sapiens
ttttattactctgggaattatgtgt

SEQ ID NO: 562 - DNA, 25 bp, Homo sapiens
tttattactctgggaattatgtgtt

SEQ ID NO: 563 - DNA, 25 bp, Homo sapiens
ttattactctgggaattatgtgttc

SEQ ID NO: 564 - DNA, 25 bp, Homo sapiens
tattactctgggaattatgtgttct

SEQ ID NO: 565 - DNA, 25 bp, Homo sapiens
attactctgggaattatgtgttctg

SEQ ID NO: 566 - DNA, 25 bp, Homo sapiens
ttactctgggaattatgtgttctgc

SEQ ID NO: 567 - DNA, 25 bp, Homo sapiens
tactctgggaattatgtgttctgcc

SEQ ID NO: 568 - DNA, 25 bp, Homo sapiens
actctgggaattatgtgttctgccc

SEQ ID NO: 569 - DNA, 25 bp, Homo sapiens
ctctgggaattatgtgttctgcccc

SEQ ID NO: 570 - DNA, 25 bp, Homo sapiens
tctgggaattatgtgttctgcccca

SEQ ID NO: 571 - DNA, 25 bp, Homo sapiens
ctgggaattatgtgttctgccccat

SEQ ID NO: 572 - DNA, 25 bp, Homo sapiens
tgggaattatgtgttctgccccatc

SEQ ID NO: 573 - DNA, 25 bp, Homo sapiens
gggaattatgtgttctgccccatca

SEQ ID NO: 574 - DNA, 25 bp, Homo sapiens
ggaattatgtgttctgccccatcac

SEQ ID NO: 575 - DNA, 25 bp, Homo sapiens
gaattatgtgttctgccccatcact

SEQ ID NO: 576 - DNA, 25 bp, Homo sapiens
aattatgtgttctgccccatcactc

SEQ ID NO: 577 - DNA, 25 bp, Homo sapiens
attatgtgttctgccccatcactct

SEQ ID NO: 578 - DNA, 25 bp, Homo sapiens
ttatgtgttctgccccatcactctc

SEQ ID NO: 579 - DNA, 25 bp, Homo sapiens
tatgtgttctgccccatcactctct

SEQ ID NO: 580 - DNA, 25 bp, Homo sapiens
atgtgttctgccccatcactctctc

SEQ ID NO: 581 - DNA, 25 bp, Homo sapiens
tgtgttctgccccatcactctctct

SEQ ID NO: 582 - DNA, 25 bp, Homo sapiens
gtgttctgccccatcactctctctt

SEQ ID NO: 583 - DNA, 25 bp, Homo sapiens
tgttctgccccatcactctctctta

SEQ ID NO: 584 - DNA, 25 bp, Homo sapiens
gttctgccccatcactctctcttaa

SEQ ID NO: 585 - DNA, 25 bp, Homo sapiens
ttctgccccatcactctctcttaat

SEQ ID NO: 586 - DNA, 25 bp, Homo sapiens
tctgccccatcactctctcttaatt

SEQ ID NO: 587 - DNA, 25 bp, Homo sapiens
ctgccccatcactctctcttaattg

SEQ ID NO: 588 - DNA, 25 bp, Homo sapiens
tgccccatcactctctcttaattgg

SEQ ID NO: 589 - DNA, 25 bp, Homo sapiens
gccccatcactctctcttaattgga

SEQ ID NO: 590 - DNA, 25 bp, Homo sapiens
ccccatcactctctcttaattggat

SEQ ID NO: 591 - DNA, 25 bp, Homo sapiens
cccatcactctctcttaattggatt

SEQ ID NO: 592 - DNA, 25 bp, Homo sapiens
ccatcactctctcttaattggattt

SEQ ID NO: 593 - DNA, 25 bp, Homo sapiens
catcactctctcttaattggatttt

SEQ ID NO: 594 - DNA, 25 bp, Homo sapiens
atcactctctcttaattggattttt

SEQ ID NO: 595 - DNA, 25 bp, Homo sapiens
tcactctctcttaattggattttta

SEQ ID NO: 596 - DNA, 25 bp, Homo sapiens
cactctctcttaattggatttttaa

SEQ ID NO: 597 - DNA, 25 bp, Homo sapiens
actctctcttaattggatttttaaa

SEQ ID NO: 598 - DNA, 25 bp, Homo sapiens
ctctctcttaattggatttttaaaa

SEQ ID NO: 599 - DNA, 25 bp, Homo sapiens
tctctcttaattggatttttaaaat

SEQ ID NO: 600 - DNA, 25 bp, Homo sapiens
ctctcttaattggatttttaaaatt

SEQ ID NO: 601 - DNA, 25 bp, Homo sapiens
tctcttaattggatttttaaaatta

SEQ ID NO: 602 - DNA, 25 bp, Homo sapiens
ctcttaattggatttttaaaattat

SEQ ID NO: 603 - DNA, 25 bp, Homo sapiens
tcttaattggatttttaaaattata

SEQ ID NO: 604 - DNA, 25 bp, Homo sapiens
cttaattggatttttaaaattatat

SEQ ID NO: 605 - DNA, 25 bp, Homo sapiens
ttaattggatttttaaaattatatt

SEQ ID NO: 606 - DNA, 25 bp, Homo sapiens
taattggatttttaaaattatattc

SEQ ID NO: 607 - DNA, 25 bp, Homo sapiens
aattggatttttaaaattatattca

SEQ ID NO: 608 - DNA, 25 bp, Homo sapiens
attggatttttaaaattatattcat

SEQ ID NO: 609 - DNA, 25 bp, Homo sapiens
ttggatttttaaaattatattcata

SEQ ID NO: 610 - DNA, 25 bp, Homo sapiens
tggatttttaaaattatattcatat

SEQ ID NO: 611 - DNA, 25 bp, Homo sapiens
ggatttttaaaattatattcatatt

SEQ ID NO: 612 - DNA, 25 bp, Homo sapiens
gatttttaaaattatattcatattg

SEQ ID NO: 613 - DNA, 25 bp, Homo sapiens
atttttaaaattatattcatattgc

SEQ ID NO: 614 - DNA, 25 bp, Homo sapiens
tttttaaaattatattcatattgca

SEQ ID NO: 615 - DNA, 25 bp, Homo sapiens
ttttaaaattatattcatattgcag

SEQ ID NO: 616 - DNA, 25 bp, Homo sapiens
tttaaaattatattcatattgcagg

SEQ ID NO: 617 - DNA, 25 bp, Homo sapiens
ttaaaattatattcatattgcagga

SEQ ID NO: 618 - DNA, 25 bp, Homo sapiens
taaaattatattcatattgcaggac

SEQ ID NO: 619 - DNA, 25 bp, Homo sapiens
aaaattatattcatattgcaggact

SEQ ID NO: 620 - DNA, 25 bp, Homo sapiens
aaattatattcatattgcaggactc

SEQ ID NO: 621 - DNA, 25 bp, Homo sapiens
aattatattcatattgcaggactcg

SEQ ID NO: 622 - DNA, 25 bp, Homo sapiens
attatattcatattgcaggactcgg

SEQ ID NO: 623 - DNA, 25 bp, Homo sapiens
ttatattcatattgcaggactcggc

SEQ ID NO: 624 - DNA, 25 bp, Homo sapiens
tatattcatattgcaggactcggca

SEQ ID NO: 625 - DNA, 25 bp, Homo sapiens
atattcatattgcaggactcggcag

SEQ ID NO: 626 - DNA, 25 bp, Homo sapiens
tattcatattgcaggactcggcaga

SEQ ID NO: 627 - DNA, 25 bp, Homo sapiens
attcatattgcaggactcggcagaa

SEQ ID NO: 628 - DNA, 25 bp, Homo sapiens
ttcatattgcaggactcggcagaag

SEQ ID NO: 629 - DNA, 25 bp, Homo sapiens
tcatattgcaggactcggcagaaga

SEQ ID NO: 630 - DNA, 25 bp, Homo sapiens
catattgcaggactcggcagaagac

SEQ ID NO: 631 - DNA, 25 bp, Homo sapiens
atattgcaggactcggcagaagacc

SEQ ID NO: 632 - DNA, 25 bp, Homo sapiens
tattgcaggactcggcagaagacct

SEQ ID NO: 633 - DNA, 25 bp, Homo sapiens
attgcaggactcggcagaagacctt

SEQ ID NO: 634 - DNA, 25 bp, Homo sapiens
ttgcaggactcggcagaagaccttc

SEQ ID NO: 635 - DNA, 25 bp, Homo sapiens
tgcaggactcggcagaagaccttcg

SEQ ID NO: 636 - DNA, 25 bp, Homo sapiens
gcaggactcggcagaagaccttcga

SEQ ID NO: 637 - DNA, 25 bp, Homo sapiens
caggactcggcagaagaccttcgag

SEQ ID NO: 638 - DNA, 25 bp, Homo sapiens
aggactcggcagaagaccttcgaga

SEQ ID NO: 639 - DNA, 25 bp, Homo sapiens
ggactcggcagaagaccttcgagag

SEQ ID NO: 640 - DNA, 25 bp, Homo sapiens
gactcggcagaagaccttcgagaga

SEQ ID NO: 641 - DNA, 25 bp, Homo sapiens
actcggcagaagaccttcgagagaa

SEQ ID NO: 642 - DNA, 25 bp, Homo sapiens
ctcggcagaagaccttcgagagaaa

SEQ ID NO: 643 - DNA, 25 bp, Homo sapiens
tcggcagaagaccttcgagagaaag

SEQ ID NO: 644 - DNA, 25 bp, Homo sapiens
cggcagaagaccttcgagagaaagg

SEQ ID NO: 645 - DNA, 25 bp, Homo sapiens
ggcagaagaccttcgagagaaaggt

SEQ ID NO: 646 - DNA, 25 bp, Homo sapiens
gcagaagaccttcgagagaaaggta

SEQ ID NO: 647 - DNA, 25 bp, Homo sapiens
cagaagaccttcgagagaaaggtag

SEQ ID NO: 648 - DNA, 25 bp, Homo sapiens
agaagaccttcgagagaaaggtaga

SEQ ID NO: 649 - DNA, 25 bp, Homo sapiens
gaagaccttcgagagaaaggtagaa

SEQ ID NO: 650 - DNA, 25 bp, Homo sapiens
aagaccttcgagagaaaggtagaaa

SEQ ID NO: 651 - DNA, 25 bp, Homo sapiens
agaccttcgagagaaaggtagaaaa

SEQ ID NO: 652 - DNA, 25 bp, Homo sapiens
gaccttcgagagaaaggtagaaaat

SEQ ID NO: 653 - DNA, 25 bp, Homo sapiens
accttcgagagaaaggtagaaaata

SEQ ID NO: 654 - DNA, 25 bp, Homo sapiens
ccttcgagagaaaggtagaaaataa

SEQ ID NO: 655 - DNA, 25 bp, Homo sapiens
cttcgagagaaaggtagaaaataag

SEQ ID NO: 656 - DNA, 25 bp, Homo sapiens
ttcgagagaaaggtagaaaataaga

SEQ ID NO: 657 - DNA, 25 bp, Homo sapiens
tcgagagaaaggtagaaaataagaa

SEQ ID NO: 658 - DNA, 25 bp, Homo sapiens
cgagagaaaggtagaaaataagaat

SEQ ID NO: 659 - DNA, 25 bp, Homo sapiens
gagagaaaggtagaaaataagaatt

SEQ ID NO: 660 - DNA, 25 bp, Homo sapiens
agagaaaggtagaaaataagaattt

SEQ ID NO: 661 - DNA, 25 bp, Homo sapiens
gagaaaggtagaaaataagaatttg

SEQ ID NO: 662 - DNA, 25 bp, Homo sapiens
agaaaggtagaaaataagaatttgg

SEQ ID NO: 663 - DNA, 25 bp, Homo sapiens
gaaaggtagaaaataagaatttggc

SEQ ID NO: 664 - DNA, 25 bp, Homo sapiens
aaaggtagaaaataagaatttggct

SEQ ID NO: 665 - DNA, 25 bp, Homo sapiens
aaggtagaaaataagaatttggctc

SEQ ID NO: 666 - DNA, 25 bp, Homo sapiens
aggtagaaaataagaatttggctct

SEQ ID NO: 667 - DNA, 25 bp, Homo sapiens
ggtagaaaataagaatttggctctc

SEQ ID NO: 668 - DNA, 25 bp, Homo sapiens
gtagaaaataagaatttggctctct

SEQ ID NO: 669 - DNA, 25 bp, Homo sapiens
tagaaaataagaatttggctctctg

SEQ ID NO: 670 - DNA, 25 bp, Homo sapiens
agaaaataagaatttggctctctgt

SEQ ID NO: 671 - DNA, 25 bp, Homo sapiens
gaaaataagaatttggctctctgtg

SEQ ID NO: 672 - DNA, 25 bp, Homo sapiens
aaaataagaatttggctctctgtgt

SEQ ID NO: 673 - DNA, 25 bp, Homo sapiens
aaataagaatttggctctctgtgtg

SEQ ID NO: 674 - DNA, 25 bp, Homo sapiens
aataagaatttggctctctgtgtga

SEQ ID NO: 675 - DNA, 25 bp, Homo sapiens
ataagaatttggctctctgtgtgag

SEQ ID NO: 676 - DNA, 25 bp, Homo sapiens
taagaatttggctctctgtgtgagc

SEQ ID NO: 677 - DNA, 25 bp, Homo sapiens
aagaatttggctctctgtgtgagca

SEQ ID NO: 678 - DNA, 25 bp, Homo sapiens
agaatttggctctctgtgtgagcat

SEQ ID NO: 679 - DNA, 25 bp, Homo sapiens
gaatttggctctctgtgtgagcatg

SEQ ID NO: 680 - DNA, 25 bp, Homo sapiens
aatttggctctctgtgtgagcatgt

SEQ ID NO: 681 - DNA, 25 bp, Homo sapiens
atttggctctctgtgtgagcatgtg

SEQ ID NO: 682 - DNA, 25 bp, Homo sapiens
tttggctctctgtgtgagcatgtgt

SEQ ID NO: 683 - DNA, 25 bp, Homo sapiens
ttggctctctgtgtgagcatgtgtg

SEQ ID NO: 684 - DNA, 25 bp, Homo sapiens
tggctctctgtgtgagcatgtgtgc

SEQ ID NO: 685 - DNA, 25 bp, Homo sapiens
ggctctctgtgtgagcatgtgtgcg

SEQ ID NO: 686 - DNA, 25 bp, Homo sapiens
gctctctgtgtgagcatgtgtgcgt

SEQ ID NO: 687 - DNA, 25 bp, Homo sapiens
ctctctgtgtgagcatgtgtgcgtg

SEQ ID NO: 688 - DNA, 25 bp, Homo sapiens
tctctgtgtgagcatgtgtgcgtgt

SEQ ID NO: 689 - DNA, 25 bp, Homo sapiens
ctctgtgtgagcatgtgtgcgtgtg

SEQ ID NO: 690 - DNA, 25 bp, Homo sapiens
tctgtgtgagcatgtgtgcgtgtgt

SEQ ID NO: 691 - DNA, 25 bp, Homo sapiens
ctgtgtgagcatgtgtgcgtgtgtg

SEQ ID NO: 692 - DNA, 25 bp, Homo sapiens
tgtgtgagcatgtgtgcgtgtgtgc

SEQ ID NO: 693 - DNA, 25 bp, Homo sapiens
gtgtgagcatgtgtgcgtgtgtgcg

SEQ ID NO: 694 - DNA, 25 bp, Homo sapiens
tgtgagcatgtgtgcgtgtgtgcga

SEQ ID NO: 695 - DNA, 25 bp, Homo sapiens
gtgagcatgtgtgcgtgtgtgcgag

SEQ ID NO: 696 - DNA, 25 bp, Homo sapiens
tgagcatgtgtgcgtgtgtgcgaga

SEQ ID NO: 697 - DNA, 25 bp, Homo sapiens
gagcatgtgtgcgtgtgtgcgagag

SEQ ID NO: 698 - DNA, 25 bp, Homo sapiens
agcatgtgtgcgtgtgtgcgagaga

SEQ ID NO: 699 - DNA, 25 bp, Homo sapiens
gcatgtgtgcgtgtgtgcgagagag

SEQ ID NO: 700 - DNA, 25 bp, Homo sapiens
catgtgtgcgtgtgtgcgagagaga

SEQ ID NO: 701 - DNA, 25 bp, Homo sapiens
atgtgtgcgtgtgtgcgagagagag

SEQ ID NO: 702 - DNA, 25 bp, Homo sapiens
tgtgtgcgtgtgtgcgagagagaga

SEQ ID NO: 703 - DNA, 25 bp, Homo sapiens
gtgtgcgtgtgtgcgagagagagag

SEQ ID NO: 704 - DNA, 25 bp, Homo sapiens
tgtgcgtgtgtgcgagagagagaga

SEQ ID NO: 705 - DNA, 25 bp, Homo sapiens
gtgcgtgtgtgcgagagagagagac

SEQ ID NO: 706 - DNA, 25 bp, Homo sapiens
tgcgtgtgtgcgagagagagagaca

SEQ ID NO: 707 - DNA, 25 bp, Homo sapiens
gcgtgtgtgcgagagagagagacag

SEQ ID NO: 708 - DNA, 25 bp, Homo sapiens
cgtgtgtgcgagagagagagacaga

SEQ ID NO: 709 - DNA, 25 bp, Homo sapiens
gtgtgtgcgagagagagagacagac

SEQ ID NO: 710 - DNA, 25 bp, Homo sapiens
tgtgtgcgagagagagagacagaca

SEQ ID NO: 711 - DNA, 25 bp, Homo sapiens
gtgtgcgagagagagagacagacag

SEQ ID NO: 712 - DNA, 25 bp, Homo sapiens
tgtgcgagagagagagacagacagc

SEQ ID NO: 713 - DNA, 25 bp, Homo sapiens
gtgcgagagagagagacagacagcc

SEQ ID NO: 714 - DNA, 25 bp, Homo sapiens
tgcgagagagagagacagacagcct

SEQ ID NO: 715 - DNA, 25 bp, Homo sapiens
gcgagagagagagacagacagcctg

SEQ ID NO: 716 - DNA, 25 bp, Homo sapiens
cgagagagagagacagacagcctgc

SEQ ID NO: 717 - DNA, 25 bp, Homo sapiens
gagagagagagacagacagcctgcc

SEQ ID NO: 718 - DNA, 25 bp, Homo sapiens
agagagagagacagacagcctgcct

SEQ ID NO: 719 - DNA, 25 bp, Homo sapiens
gagagagagacagacagcctgccta

SEQ ID NO: 720 - DNA, 25 bp, Homo sapiens
agagagagacagacagcctgcctaa

SEQ ID NO: 721 - DNA, 25 bp, Homo sapiens
gagagagacagacagcctgcctaag

SEQ ID NO: 722 - DNA, 25 bp, Homo sapiens
agagagacagacagcctgcctaaga

SEQ ID NO: 723 - DNA, 25 bp, Homo sapiens
gagagacagacagcctgcctaagaa

SEQ ID NO: 724 - DNA, 25 bp, Homo sapiens
agagacagacagcctgcctaagaag

SEQ ID NO: 725 - DNA, 25 bp, Homo sapiens
gagacagacagcctgcctaagaaga

SEQ ID NO: 726 - DNA, 25 bp, Homo sapiens
agacagacagcctgcctaagaagaa

SEQ ID NO: 727 - DNA, 25 bp, Homo sapiens
gacagacagcctgcctaagaagaaa

SEQ ID NO: 728 - DNA, 25 bp, Homo sapiens
acagacagcctgcctaagaagaaat

SEQ ID NO: 729 - DNA, 25 bp, Homo sapiens
cagacagcctgcctaagaagaaatg

SEQ ID NO: 730 - DNA, 25 bp, Homo sapiens
agacagcctgcctaagaagaaatga

SEQ ID NO: 731 - DNA, 25 bp, Homo sapiens
gacagcctgcctaagaagaaatgaa

SEQ ID NO: 732 - DNA, 25 bp, Homo sapiens
acagcctgcctaagaagaaatgaat

SEQ ID NO: 733 - DNA, 25 bp, Homo sapiens
cagcctgcctaagaagaaatgaatg

SEQ ID NO: 734 - DNA, 25 bp, Homo sapiens
agcctgcctaagaagaaatgaatgt

SEQ ID NO: 735 - DNA, 25 bp, Homo sapiens
gcctgcctaagaagaaatgaatgtg

SEQ ID NO: 736 - DNA, 25 bp, Homo sapiens
cctgcctaagaagaaatgaatgtga

SEQ ID NO: 737 - DNA, 25 bp, Homo sapiens
ctgcctaagaagaaatgaatgtgaa

SEQ ID NO: 738 - DNA, 25 bp, Homo sapiens
tgcctaagaagaaatgaatgtgaat

SEQ ID NO: 739 - DNA, 25 bp, Homo sapiens
gcctaagaagaaatgaatgtgaatg

SEQ ID NO: 740 - DNA, 25 bp, Homo sapiens
cctaagaagaaatgaatgtgaatgc

SEQ ID NO: 741 - DNA, 25 bp, Homo sapiens
ctaagaagaaatgaatgtgaatgcg

SEQ ID NO: 742 - DNA, 25 bp, Homo sapiens
taagaagaaatgaatgtgaatgcgg

SEQ ID NO: 743 - DNA, 25 bp, Homo sapiens
aagaagaaatgaatgtgaatgcggc

SEQ ID NO: 744 - DNA, 25 bp, Homo sapiens
agaagaaatgaatgtgaatgcggct

SEQ ID NO: 745 - DNA, 25 bp, Homo sapiens
gaagaaatgaatgtgaatgcggctt

SEQ ID NO: 746 - DNA, 25 bp, Homo sapiens
aagaaatgaatgtgaatgcggcttg

SEQ ID NO: 747 - DNA, 25 bp, Homo sapiens
agaaatgaatgtgaatgcggcttgt

SEQ ID NO: 748 - DNA, 25 bp, Homo sapiens
gaaatgaatgtgaatgcggcttgtg

SEQ ID NO: 749 - DNA, 25 bp, Homo sapiens
aaatgaatgtgaatgcggcttgtgg

SEQ ID NO: 750 - DNA, 25 bp, Homo sapiens
aatgaatgtgaatgcggcttgtggc

SEQ ID NO: 751 - DNA, 25 bp, Homo sapiens
atgaatgtgaatgcggcttgtggca

SEQ ID NO: 752 - DNA, 25 bp, Homo sapiens
tgaatgtgaatgcggcttgtggcac

SEQ ID NO: 753 - DNA, 25 bp, Homo sapiens
gaatgtgaatgcggcttgtggcaca

SEQ ID NO: 754 - DNA, 25 bp, Homo sapiens
aatgtgaatgcggcttgtggcacag

SEQ ID NO: 755 - DNA, 25 bp, Homo sapiens
atgtgaatgcggcttgtggcacagt

SEQ ID NO: 756 - DNA, 25 bp, Homo sapiens
tgtgaatgcggcttgtggcacagtt

SEQ ID NO: 757 - DNA, 25 bp, Homo sapiens
gtgaatgcggcttgtggcacagttg

SEQ ID NO: 758 - DNA, 25 bp, Homo sapiens
tgaatgcggcttgtggcacagttga

SEQ ID NO: 759 - DNA, 25 bp, Homo sapiens
gaatgcggcttgtggcacagttgac

SEQ ID NO: 760 - DNA, 25 bp, Homo sapiens
aatgcggcttgtggcacagttgaca

SEQ ID NO: 761 - DNA, 25 bp, Homo sapiens
atgcggcttgtggcacagttgacaa

SEQ ID NO: 762 - DNA, 25 bp, Homo sapiens
tgcggcttgtggcacagttgacaag

SEQ ID NO: 763 - DNA, 25 bp, Homo sapiens
gcggcttgtggcacagttgacaagg

SEQ ID NO: 764 - DNA, 25 bp, Homo sapiens
cggcttgtggcacagttgacaagga

SEQ ID NO: 765 - DNA, 25 bp, Homo sapiens
ggcttgtggcacagttgacaaggat

SEQ ID NO: 766 - DNA, 25 bp, Homo sapiens
gcttgtggcacagttgacaaggatg

SEQ ID NO: 767 - DNA, 25 bp, Homo sapiens
cttgtggcacagttgacaaggatga

SEQ ID NO: 768 - DNA, 25 bp, Homo sapiens
ttgtggcacagttgacaaggatgat

SEQ ID NO: 769 - DNA, 25 bp, Homo sapiens
tgtggcacagttgacaaggatgata

SEQ ID NO: 770 - DNA, 25 bp, Homo sapiens
gtggcacagttgacaaggatgataa

SEQ ID NO: 771 - DNA, 25 bp, Homo sapiens
tggcacagttgacaaggatgataaa

SEQ ID NO: 772 - DNA, 25 bp, Homo sapiens
ggcacagttgacaaggatgataaat

SEQ ID NO: 773 - DNA, 25 bp, Homo sapiens
gcacagttgacaaggatgataaatc

SEQ ID NO: 774 - DNA, 25 bp, Homo sapiens
cacagttgacaaggatgataaatca

SEQ ID NO: 775 - DNA, 25 bp, Homo sapiens
acagttgacaaggatgataaatcaa

SEQ ID NO: 776 - DNA, 25 bp, Homo sapiens
cagttgacaaggatgataaatcaat

SEQ ID NO: 777 - DNA, 25 bp, Homo sapiens
agttgacaaggatgataaatcaata

SEQ ID NO: 778 - DNA, 25 bp, Homo sapiens
gttgacaaggatgataaatcaataa

SEQ ID NO: 779 - DNA, 25 bp, Homo sapiens
ttgacaaggatgataaatcaataat

SEQ ID NO: 780 - DNA, 25 bp, Homo sapiens
tgacaaggatgataaatcaataatg

SEQ ID NO: 781 - DNA, 25 bp, Homo sapiens
gacaaggatgataaatcaataatgc

SEQ ID NO: 782 - DNA, 25 bp, Homo sapiens
acaaggatgataaatcaataatgca

SEQ ID NO: 783 - DNA, 25 bp, Homo sapiens
caaggatgataaatcaataatgcaa

SEQ ID NO: 784 - DNA, 25 bp, Homo sapiens
aaggatgataaatcaataatgcaag

SEQ ID NO: 785 - DNA, 25 bp, Homo sapiens
aggatgataaatcaataatgcaagc

SEQ ID NO: 786 - DNA, 25 bp, Homo sapiens
ggatgataaatcaataatgcaagct

SEQ ID NO: 787 - DNA, 25 bp, Homo sapiens
gatgataaatcaataatgcaagctt

SEQ ID NO: 788 - DNA, 25 bp, Homo sapiens
atgataaatcaataatgcaagctta

SEQ ID NO: 789 - DNA, 25 bp, Homo sapiens
tgataaatcaataatgcaagcttac

SEQ ID NO: 790 - DNA, 25 bp, Homo sapiens
gataaatcaataatgcaagcttact

SEQ ID NO: 791 - DNA, 25 bp, Homo sapiens
ataaatcaataatgcaagcttacta

SEQ ID NO: 792 - DNA, 25 bp, Homo sapiens
taaatcaataatgcaagcttactat

SEQ ID NO: 793 - DNA, 25 bp, Homo sapiens
aaatcaataatgcaagcttactatc

SEQ ID NO: 794 - DNA, 25 bp, Homo sapiens
aatcaataatgcaagcttactatca

SEQ ID NO: 795 - DNA, 25 bp, Homo sapiens
atcaataatgcaagcttactatcat

SEQ ID NO: 796 - DNA, 25 bp, Homo sapiens
tcaataatgcaagcttactatcatt

SEQ ID NO: 797 - DNA, 25 bp, Homo sapiens
caataatgcaagcttactatcattt

SEQ ID NO: 798 - DNA, 25 bp, Homo sapiens
aataatgcaagcttactatcattta

SEQ ID NO: 799 - DNA, 25 bp, Homo sapiens
ataatgcaagcttactatcatttat

SEQ ID NO: 800 - DNA, 25 bp, Homo sapiens
taatgcaagcttactatcatttatg

SEQ ID NO: 801 - DNA, 25 bp, Homo sapiens
aatgcaagcttactatcatttatga

SEQ ID NO: 802 - DNA, 25 bp, Homo sapiens
atgcaagcttactatcatttatgaa

SEQ ID NO: 803 - DNA, 25 bp, Homo sapiens
tgcaagcttactatcatttatgaat

SEQ ID NO: 804 - DNA, 25 bp, Homo sapiens
gcaagcttactatcatttatgaata

SEQ ID NO: 805 - DNA, 25 bp, Homo sapiens
caagcttactatcatttatgaatag

SEQ ID NO: 806 - DNA, 25 bp, Homo sapiens
aagcttactatcatttatgaatagc

SEQ ID NO: 807 - DNA, 25 bp, Homo sapiens
agcttactatcatttatgaatagca

SEQ ID NO: 808 - DNA, 25 bp, Homo sapiens
gcttactatcatttatgaatagcaa

SEQ ID NO: 809 - DNA, 25 bp, Homo sapiens
cttactatcatttatgaatagcaat

SEQ ID NO: 810 - DNA, 25 bp, Homo sapiens
ttactatcatttatgaatagcaata

SEQ ID NO: 811 - DNA, 25 bp, Homo sapiens
tactatcatttatgaatagcaatac

SEQ ID NO: 812 - DNA, 25 bp, Homo sapiens
actatcatttatgaatagcaatact

SEQ ID NO: 813 - DNA, 25 bp, Homo sapiens
ctatcatttatgaatagcaatactg

SEQ ID NO: 814 - DNA, 25 bp, Homo sapiens
tatcatttatgaatagcaatactga

SEQ ID NO: 815 - DNA, 25 bp, Homo sapiens
atcatttatgaatagcaatactgaa

SEQ ID NO: 816 - DNA, 25 bp, Homo sapiens
tcatttatgaatagcaatactgaag

SEQ ID NO: 817 - DNA, 25 bp, Homo sapiens
catttatgaatagcaatactgaaga

SEQ ID NO: 818 - DNA, 25 bp, Homo sapiens
atttatgaatagcaatactgaagaa

SEQ ID NO: 819 - DNA, 25 bp, Homo sapiens
tttatgaatagcaatactgaagaaa

SEQ ID NO: 820 - DNA, 25 bp, Homo sapiens
ttatgaatagcaatactgaagaaat

SEQ ID NO: 821 - DNA, 25 bp, Homo sapiens
tatgaatagcaatactgaagaaatt

SEQ ID NO: 822 - DNA, 25 bp, Homo sapiens
atgaatagcaatactgaagaaatta

SEQ ID NO: 823 - DNA, 25 bp, Homo sapiens
tgaatagcaatactgaagaaattaa

SEQ ID NO: 824 - DNA, 25 bp, Homo sapiens
gaatagcaatactgaagaaattaaa

SEQ ID NO: 825 - DNA, 25 bp, Homo sapiens
aatagcaatactgaagaaattaaaa

SEQ ID NO: 826 - DNA, 25 bp, Homo sapiens
atagcaatactgaagaaattaaaac

SEQ ID NO: 827 - DNA, 25 bp, Homo sapiens
tagcaatactgaagaaattaaaaca

SEQ ID NO: 828 - DNA, 25 bp, Homo sapiens
agcaatactgaagaaattaaaacaa

SEQ ID NO: 829 - DNA, 25 bp, Homo sapiens
gcaatactgaagaaattaaaacaaa

SEQ ID NO: 830 - DNA, 25 bp, Homo sapiens
caatactgaagaaattaaaacaaaa

SEQ ID NO: 831 - DNA, 25 bp, Homo sapiens
aatactgaagaaattaaaacaaaag

SEQ ID NO: 832 - DNA, 25 bp, Homo sapiens
atactgaagaaattaaaacaaaaga

SEQ ID NO: 833 - DNA, 25 bp, Homo sapiens
tactgaagaaattaaaacaaaagat

SEQ ID NO: 834 - DNA, 25 bp, Homo sapiens
actgaagaaattaaaacaaaagatt

SEQ ID NO: 835 - DNA, 25 bp, Homo sapiens
ctgaagaaattaaaacaaaagattg

SEQ ID NO: 836 - DNA, 25 bp, Homo sapiens
tgaagaaattaaaacaaaagattgc

SEQ ID NO: 837 - DNA, 25 bp, Homo sapiens
gaagaaattaaaacaaaagattgct

SEQ ID NO: 838 - DNA, 25 bp, Homo sapiens
aagaaattaaaacaaaagattgctg

SEQ ID NO: 839 - DNA, 25 bp, Homo sapiens
agaaattaaaacaaaagattgctgt

SEQ ID NO: 840 - DNA, 25 bp, Homo sapiens
gaaattaaaacaaaagattgctgtc

SEQ ID NO: 841 - DNA, 25 bp, Homo sapiens
aaattaaaacaaaagattgctgtct

SEQ ID NO: 842 - DNA, 25 bp, Homo sapiens
aattaaaacaaaagattgctgtctc

SEQ ID NO: 843 - DNA, 25 bp, Homo sapiens
attaaaacaaaagattgctgtctca

SEQ ID NO: 844 - DNA, 25 bp, Homo sapiens
ttaaaacaaaagattgctgtctcaa

SEQ ID NO: 845 - DNA, 25 bp, Homo sapiens
taaaacaaaagattgctgtctcaat

SEQ ID NO: 846 - DNA, 25 bp, Homo sapiens
aaaacaaaagattgctgtctcaata

SEQ ID NO: 847 - DNA, 25 bp, Homo sapiens
aaacaaaagattgctgtctcaatat

SEQ ID NO: 848 - DNA, 25 bp, Homo sapiens
aacaaaagattgctgtctcaatata

SEQ ID NO: 849 - DNA, 25 bp, Homo sapiens
acaaaagattgctgtctcaatatat

SEQ ID NO: 850 - DNA, 25 bp, Homo sapiens
caaaagattgctgtctcaatatatc

SEQ ID NO: 851 - DNA, 25 bp, Homo sapiens
aaaagattgctgtctcaatatatct

SEQ ID NO: 852 - DNA, 25 bp, Homo sapiens
aaagattgctgtctcaatatatctt

SEQ ID NO: 853 - DNA, 25 bp, Homo sapiens
aagattgctgtctcaatatatctta

SEQ ID NO: 854 - DNA, 25 bp, Homo sapiens
agattgctgtctcaatatatcttat

SEQ ID NO: 855 - DNA, 25 bp, Homo sapiens
gattgctgtctcaatatatcttata

SEQ ID NO: 856 - DNA, 25 bp, Homo sapiens
attgctgtctcaatatatcttatat

SEQ ID NO: 857 - DNA, 25 bp, Homo sapiens
ttgctgtctcaatatatcttatatt

SEQ ID NO: 858 - DNA, 25 bp, Homo sapiens
tgctgtctcaatatatcttatattt

SEQ ID NO: 859 - DNA, 25 bp, Homo sapiens
gctgtctcaatatatcttatattta

SEQ ID NO: 860 - DNA, 25 bp, Homo sapiens
ctgtctcaatatatcttatatttat

SEQ ID NO: 861 - DNA, 25 bp, Homo sapiens
tgtctcaatatatcttatatttatt

SEQ ID NO: 862 - DNA, 25 bp, Homo sapiens
gtctcaatatatcttatatttatta

SEQ ID NO: 863 - DNA, 25 bp, Homo sapiens
tctcaatatatcttatatttattat

SEQ ID NO: 864 - DNA, 25 bp, Homo sapiens
ctcaatatatcttatatttattatt

SEQ ID NO: 865 - DNA, 25 bp, Homo sapiens
tcaatatatcttatatttattattt

SEQ ID NO: 866 - DNA, 25 bp, Homo sapiens
caatatatcttatatttattattta

SEQ ID NO: 867 - DNA, 25 bp, Homo sapiens
aatatatcttatatttattatttac

SEQ ID NO: 868 - DNA, 25 bp, Homo sapiens
atatatcttatatttattatttacc

SEQ ID NO: 869 - DNA, 25 bp, Homo sapiens
tatatcttatatttattatttacca

SEQ ID NO: 870 - DNA, 25 bp, Homo sapiens
atatcttatatttattatttaccaa

SEQ ID NO: 871 - DNA, 25 bp, Homo sapiens
tatcttatatttattatttaccaaa

SEQ ID NO: 872 - DNA, 25 bp, Homo sapiens
atcttatatttattatttaccaaat

SEQ ID NO: 873 - DNA, 25 bp, Homo sapiens
tcttatatttattatttaccaaatt

SEQ ID NO: 874 - DNA, 25 bp, Homo sapiens
cttatatttattatttaccaaatta

SEQ ID NO: 875 - DNA, 25 bp, Homo sapiens
ttatatttattatttaccaaattat

SEQ ID NO: 876 - DNA, 25 bp, Homo sapiens
tatatttattatttaccaaattatt

SEQ ID NO: 877 - DNA, 25 bp, Homo sapiens
atatttattatttaccaaattattc

SEQ ID NO: 878 - DNA, 25 bp, Homo sapiens
tatttattatttaccaaattattct

SEQ ID NO: 879 - DNA, 25 bp, Homo sapiens
atttattatttaccaaattattcta

SEQ ID NO: 880 - DNA, 25 bp, Homo sapiens
tttattatttaccaaattattctaa

SEQ ID NO: 881 - DNA, 25 bp, Homo sapiens
ttattatttaccaaattattctaag

SEQ ID NO: 882 - DNA, 25 bp, Homo sapiens
tattatttaccaaattattctaaga

SEQ ID NO: 883 - DNA, 25 bp, Homo sapiens
attatttaccaaattattctaagag

SEQ ID NO: 884 - DNA, 25 bp, Homo sapiens
ttatttaccaaattattctaagagt

SEQ ID NO: 885 - DNA, 25 bp, Homo sapiens
tatttaccaaattattctaagagta

SEQ ID NO: 886 - DNA, 25 bp, Homo sapiens
atttaccaaattattctaagagtat

SEQ ID NO: 887 - DNA, 25 bp, Homo sapiens
tttaccaaattattctaagagtatt

SEQ ID NO: 888 - DNA, 25 bp, Homo sapiens
ttaccaaattattctaagagtattt

SEQ ID NO: 889 - DNA, 25 bp, Homo sapiens
taccaaattattctaagagtatttc

SEQ ID NO: 890 - DNA, 25 bp, Homo sapiens
accaaattattctaagagtatttct

SEQ ID NO: 891 - DNA, 25 bp, Homo sapiens
ccaaattattctaagagtatttctt

SEQ ID NO: 892 - DNA, 25 bp, Homo sapiens
caaattattctaagagtatttcttc

SEQ ID NO: 893 - DNA, 25 bp, Homo sapiens
aaattattctaagagtatttcttcc

SEQ ID NO: 894 - DNA, 23 bp, Artificial Sequence
  Description of Artificial Sequence Synthetic oligonucleotide
atccaattaagagagagtgatgg

SEQ ID NO: 895 - DNA, 23 bp, Artificial Sequence
  Description of Artificial Sequence Synthetic oligonucleotide
aatccaattaagagagagtgatg

SEQ ID NO: 896 - DNA, 23 bp, Artificial Sequence
  Description of Artificial Sequence Synthetic oligonucleotide
tccaattaagagagagtgatggg

SEQ ID NO: 897 - DNA, 21 bp, Artificial Sequence
  Description of Artificial Sequence Synthetic oligonucleotide
tccaattaagagagagtgatg

SEQ ID NO: 898 - DNA, 19 bp, Artificial Sequence
  Description of Artificial Sequence Synthetic oligonucleotide
ccaattaagagagagtgat

SEQ ID NO: 899 - DNA, 21 bp, Artificial Sequence
  Description of Artificial Sequence Synthetic oligonucleotide
aatccaattaagagagagtga

SEQ ID NO: 900 - DNA, 21 bp, Artificial Sequence
  Description of Artificial Sequence Synthetic oligonucleotide
caattaagagagagtgatggg

SEQ ID NO: 901 - DNA, 23 bp, Artificial Sequence
  Description of Artificial Sequence Synthetic oligonucleotide
gagtcctgcaatatgaatataat

SEQ ID NO: 902 - DNA, 23 bp, Artificial Sequence
  Description of Artificial Sequence Synthetic oligonucleotide
gtcctgcaatatgaatataattt

SEQ ID NO: 903 - DNA, 21 bp, Artificial Sequence
  Description of Artificial Sequence Synthetic oligonucleotide
gtcctgcaatatgaatataat

SEQ ID NO: 904 - DNA, 21 bp, Artificial Sequence
  Description of Artificial Sequence Synthetic oligonucleotide
gagtcctgcaatatgaatata

SEQ ID NO: 905 - DNA, 21 bp, Artificial Sequence
  Description of Artificial Sequence Synthetic oligonucleotide
cctgcaatatgaatataattt

SEQ ID NO: 906 - DNA, 19 bp, Artificial Sequence
  Description of Artificial Sequence Synthetic oligonucleotide
gagtcctgcaatatgaata

SEQ ID NO: 907 - DNA, 19 bp, Artificial Sequence
  Description of Artificial Sequence Synthetic oligonucleotide
tgcaatatgaatataattt

SEQ ID NO: 908 - DNA, 23 bp, Artificial Sequence
  Description of Artificial Sequence Synthetic oligonucleotide
gtcttctgccgagtcctgcaata

SEQ ID NO: 909 - DNA, 21 bp, Artificial Sequence
  Description of Artificial Sequence Synthetic oligonucleotide
aggtcttctgccgagtcctgc

SEQ ID NO: 910 - DNA, 21 bp, Artificial Sequence
  Description of Artificial Sequence Synthetic oligonucleotide
cttctgccgagtcctgcaata

SEQ ID NO: 911 - DNA, 19 bp, Artificial Sequence
  Description of Artificial Sequence Synthetic oligonucleotide
tctgccgagtcctgcaata

SEQ ID NO: 912 - DNA, 23 bp, Artificial Sequence
  Description of Artificial Sequence Synthetic oligonucleotide
gcacacatgctcacacagagagc

SEQ ID NO: 913 - DNA, 23 bp, Artificial Sequence
  Description of Artificial Sequence Synthetic oligonucleotide
acacatgctcacacagagagcca

SEQ ID NO: 914 - DNA, 21 bp, Artificial Sequence
  Description of Artificial Sequence Synthetic oligonucleotide
acacatgctcacacagagagc

SEQ ID NO: 915 - DNA, 21 bp, Artificial Sequence
  Description of Artificial Sequence Synthetic oligonucleotide
gcacacatgctcacacagaga

SEQ ID NO: 916 - DNA, 21 bp, Artificial Sequence
  Description of Artificial Sequence Synthetic oligonucleotide
acatgctcacacagagagcca

SEQ ID NO: 917 - DNA, 19 bp, Artificial Sequence
  Description of Artificial Sequence Synthetic oligonucleotide
gcacacatgctcacacaga

SEQ ID NO: 918 - DNA, 19 bp, Artificial Sequence
  Description of Artificial Sequence Synthetic oligonucleotide
atgctcacacagagagcca

SEQ ID NO: 919 - DNA, 23 bp, Homo sapiens
ccatcactctctcttaattggat

SEQ ID NO: 920 - DNA, 23 bp, Homo sapiens
catcactctctcttaattggatt

SEQ ID NO: 921 - DNA, 23 bp, Homo sapiens
cccatcactctctcttaattgga

SEQ ID NO: 922 - DNA, 21 bp, Homo sapiens
catcactctctcttaattgga

SEQ ID NO: 923 - DNA, 19 bp, Homo sapiens
atcactctctcttaattgg

SEQ ID NO: 924 - DNA, 21 bp, Homo sapiens
tcactctctcttaattggatt

SEQ ID NO: 925 - DNA, 21 bp, Homo sapiens
cccatcactctctcttaattg

SEQ ID NO: 926 - DNA, 23 bp, Homo sapiens
attatattcatattgcaggactc

SEQ ID NO: 927 - DNA, 23 bp, Homo sapiens
aaattatattcatattgcaggac

SEQ ID NO: 928 - DNA, 21 bp, Homo sapiens
attatattcatattgcaggac

SEQ ID NO: 929 - DNA, 21 bp, Homo sapiens
tatattcatattgcaggactc

SEQ ID NO: 930 - DNA, 21 bp, Homo sapiens
aaattatattcatattgcagg

SEQ ID NO: 931 - DNA, 19 bp, Homo sapiens
tattcatattgcaggactc

SEQ ID NO: 932 - DNA, 19 bp, Homo sapiens
aaattatattcatattgca

SEQ ID NO: 933 - DNA, 23 bp, Homo sapiens
tattgcaggactcggcagaagac

SEQ ID NO: 934 - DNA, 21 bp, Homo sapiens
gcaggactcggcagaagacct

SEQ ID NO: 935 - DNA, 21 bp, Homo sapiens
tattgcaggactcggcagaag

SEQ ID NO: 936 - DNA, 19 bp, Homo sapiens
tattgcaggactcggcaga

SEQ ID NO: 937 - DNA, 23 bp, Homo sapiens
gctctctgtgtgagcatgtgtgc

SEQ ID NO: 938 - DNA, 23 bp, Homo sapiens
tggctctctgtgtgagcatgtgt

SEQ ID NO: 939 - DNA, 21 bp, Homo sapiens
gctctctgtgtgagcatgtgt

SEQ ID NO: 940 - DNA, 21 bp, Homo sapiens
tctctgtgtgagcatgtgtgc

SEQ ID NO: 941 - DNA, 21 bp, Homo sapiens
tggctctctgtgtgagcatgt

SEQ ID NO: 942 - DNA, 19 bp, Homo sapiens
tctgtgtgagcatgtgtgc

SEQ ID NO: 943 - DNA, 19 bp, Homo sapiens
tggctctctgtgtgagcat

SEQ ID NO: 944 - DNA, 470 bp, Homo sapiens
acttgtaatatacaggtatccctcctggtaagctctggtattatgtcttaacatttttaaatctatggtaatctttacaaaatattttacttccgaactcatatacctggggattttattactctgggaattatgtgttctgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttatatttattatttaccaaattattctaagagtatttcttcc

SEQ ID NO: 945 - RNA, 25 nt, Artificial Sequence
  Description of Artificial Sequence Synthetic oligonucleotide
ggagggauaccuguauauuacaagu

SEQ ID NO: 946 - RNA, 25 nt, Artificial Sequence
  Description of Artificial Sequence Synthetic oligonucleotide
aggagggauaccuguauauuacaag

SEQ ID NO: 947 - RNA, 25 nt, Artificial Sequence
  Description of Artificial Sequence Synthetic oligonucleotide
caggagggauaccuguauauuacaa

SEQ ID NO: 948 - RNA, 25 nt, Artificial Sequence
  Description of Artificial Sequence Synthetic oligonucleotide
ccaggagggauaccuguauauuaca

SEQ ID NO: 949 - RNA, 25 nt, Artificial Sequence
  Description of Artificial Sequence Synthetic oligonucleotide
accaggagggauaccuguauauuac

SEQ ID NO: 950 - RNA, 25 nt, Artificial Sequence
  Description of Artificial Sequence Synthetic oligonucleotide
uaccaggagggauaccuguauauua

SEQ ID NO: 951 - RNA, 25 nt, Artificial Sequence
  Description of Artificial Sequence Synthetic oligonucleotide
uuaccaggagggauaccuguauauu

SEQ ID NO: 952 - RNA, 25 nt, Artificial Sequence
  Description of Artificial Sequence Synthetic oligonucleotide
cuuaccaggagggauaccuguauau

SEQ ID NO: 953 - RNA, 25 nt, Artificial Sequence
  Description of Artificial Sequence Synthetic oligonucleotide
gcuuaccaggagggauaccuguaua

SEQ ID NO: 954 - RNA, 25 nt, Artificial Sequence
  Description of Artificial Sequence Synthetic oligonucleotide
agcuuaccaggagggauaccuguau

SEQ ID NO: 955 - RNA, 25 nt, Artificial Sequence
  Description of Artificial Sequence Synthetic oligonucleotide
gagcuuaccaggagggauaccugua

SEQ ID NO: 956 - RNA, 25 nt, Artificial Sequence
  Description of Artificial Sequence Synthetic oligonucleotide
agagcuuaccaggagggauaccugu

SEQ ID NO: 957 - RNA, 25 nt, Artificial Sequence
  Description of Artificial Sequence Synthetic oligonucleotide
cagagcuuaccaggagggauaccug

SEQ ID NO: 958 - RNA, 25 nt, Artificial Sequence
  Description of Artificial Sequence Synthetic oligonucleotide
ccagagcuuaccaggagggauaccu

SEQ ID NO: 959 - RNA, 25 nt, Artificial Sequence
  Description of Artificial Sequence Synthetic oligonucleotide
accagagcuuaccaggagggauacc

SEQ ID NO: 960 - RNA, 25 nt, Artificial Sequence
  Description of Artificial Sequence Synthetic oligonucleotide
uaccagagcuuaccaggagggauac

SEQ ID NO: 961 - RNA, 25 nt, Artificial Sequence
  Description of Artificial Sequence Synthetic oligonucleotide
auaccagagcuuaccaggagggaua

SEQ ID NO: 962 - RNA, 25 nt, Artificial Sequence
  Description of Artificial Sequence Synthetic oligonucleotide
aauaccagagcuuaccaggagggau

SEQ ID NO: 963 - RNA, 25 nt, Artificial Sequence
  Description of Artificial Sequence Synthetic oligonucleotide
uaauaccagagcuuaccaggaggga

SEQ ID NO: 964 - RNA, 25 nt, Artificial Sequence
  Description of Artificial Sequence Synthetic oligonucleotide
auaauaccagagcuuaccaggaggg

SEQ ID NO: 965 - RNA, 25 nt, Artificial Sequence
  Description of Artificial Sequence Synthetic oligonucleotide
cauaauaccagagcuuaccaggagg

SEQ ID NO: 966 - RNA, 25 nt, Artificial Sequence
  Description of Artificial Sequence Synthetic oligonucleotide
acauaauaccagagcuuaccaggag

SEQ ID NO: 967 - RNA, 25 nt, Artificial Sequence
  Description of Artificial Sequence Synthetic oligonucleotide
gacauaauaccagagcuuaccagga

SEQ ID NO: 968 - RNA, 25 nt, Artificial Sequence
  Description of Artificial Sequence Synthetic oligonucleotide
agacauaauaccagagcuuaccagg

SEQ ID NO: 969 - RNA, 25 nt, Artificial Sequence
  Description of Artificial Sequence Synthetic oligonucleotide
aagacauaauaccagagcuuaccag

SEQ ID NO: 970 - RNA, 25 nt, Artificial Sequence
  Description of Artificial Sequence Synthetic oligonucleotide
uaagacauaauaccagagcuuacca

SEQ ID NO: 971 - RNA, 25 nt, Artificial Sequence
  Description of Artificial Sequence Synthetic oligonucleotide
uuaagacauaauaccagagcuuacc

SEQ ID NO: 972 - RNA, 25 nt, Artificial Sequence
  Description of Artificial Sequence Synthetic oligonucleotide
guuaagacauaauaccagagcuuac

SEQ ID NO: 973 - RNA, 25 nt, Artificial Sequence
  Description of Artificial Sequence Synthetic oligonucleotide
uguuaagacauaauaccagagcuua

SEQ ID NO: 974 - RNA, 25 nt, Artificial Sequence
  Description of Artificial Sequence Synthetic oligonucleotide
auguuaagacauaauaccagagcuu

SEQ ID NO: 975 - RNA, 25 nt, Artificial Sequence
  Description of Artificial Sequence Synthetic oligonucleotide
aauguuaagacauaauaccagagcu

SEQ ID NO: 976 - RNA, 25 nt, Artificial Sequence
  Description of Artificial Sequence Synthetic oligonucleotide
aaauguuaagacauaauaccagagc

SEQ ID NO: 977 - RNA, 25 nt, Artificial Sequence
  Description of Artificial Sequence Synthetic oligonucleotide
aaaauguuaagacauaauaccagag

SEQ ID NO: 978 - RNA, 25 nt, Artificial Sequence
  Description of Artificial Sequence Synthetic oligonucleotide
aaaaauguuaagacauaauaccaga

SEQ ID NO: 979 - RNA, 25 nt, Artificial Sequence
  Description of Artificial Sequence Synthetic oligonucleotide
uaaaaauguuaagacauaauaccag

SEQ ID NO: 980 - RNA, 25 nt, Artificial Sequence
  Description of Artificial Sequence Synthetic oligonucleotide
uuaaaaauguuaagacauaauacca

SEQ ID NO: 981 - RNA, 25 nt, Artificial Sequence
  Description of Artificial Sequence Synthetic oligonucleotide
uuuaaaaauguuaagacauaauacc

SEQ ID NO: 982 - RNA, 25 nt, Artificial Sequence
  Description of Artificial Sequence Synthetic oligonucleotide
auuuaaaaauguuaagacauaauac

SEQ ID NO: 983 - RNA, 25 nt, Artificial Sequence
  Description of Artificial Sequence Synthetic oligonucleotide
gauuuaaaaauguuaagacauaaua

SEQ ID NO: 984 - RNA, 25 nt, Artificial Sequence
  Description of Artificial Sequence Synthetic oligonucleotide
agauuuaaaaauguuaagacauaau

SEQ ID NO: 985 - RNA, 25 nt, Artificial Sequence
  Description of Artificial Sequence Synthetic oligonucleotide
uagauuuaaaaauguuaagacauaa

SEQ ID NO: 986 - RNA, 25 nt, Artificial Sequence
  Description of Artificial Sequence Synthetic oligonucleotide
auagauuuaaaaauguuaagacaua

SEQ ID NO: 987 - RNA, 25 nt, Artificial Sequence
  Description of Artificial Sequence Synthetic oligonucleotide
cauagauuuaaaaauguuaagacau

SEQ ID NO: 988 - RNA, 25 nt, Artificial Sequence
  Description of Artificial Sequence Synthetic oligonucleotide
ccauagauuuaaaaauguuaagaca

SEQ ID NO: 989 - RNA, 25 nt, Artificial Sequence
  Description of Artificial Sequence Synthetic oligonucleotide
accauagauuuaaaaauguuaagac

SEQ ID NO: 990 - RNA, 25 nt, Artificial Sequence
  Description of Artificial Sequence Synthetic oligonucleotide
uaccauagauuuaaaaauguuaaga

SEQ ID NO: 991 - RNA, 25 nt, Artificial Sequence
  Description of Artificial Sequence Synthetic oligonucleotide
uuaccauagauuuaaaaauguuaag

SEQ ID NO: 992 - RNA, 25 nt, Artificial Sequence
  Description of Artificial Sequence Synthetic oligonucleotide
auuaccauagauuuaaaaauguuaa

SEQ ID NO: 993 - RNA, 25 nt, Artificial Sequence
  Description of Artificial Sequence Synthetic oligonucleotide
gauuaccauagauuuaaaaauguua

SEQ ID NO: 994 - RNA, 25 nt, Artificial Sequence
  Description of Artificial Sequence Synthetic oligonucleotide
agauuaccauagauuuaaaaauguu

SEQ ID NO: 995 - RNA, 25 nt, Artificial Sequence
  Description of Artificial Sequence Synthetic oligonucleotide
aagauuaccauagauuuaaaaaugu

SEQ ID NO: 996 - RNA, 25 nt, Artificial Sequence
  Description of Artificial Sequence Synthetic oligonucleotide
aaagauuaccauagauuuaaaaaug

SEQ ID NO: 997 - RNA, 25 nt, Artificial Sequence
  Description of Artificial Sequence Synthetic oligonucleotide
uaaagauuaccauagauuuaaaaau

SEQ ID NO: 998 - RNA, 25 nt, Artificial Sequence
  Description of Artificial Sequence Synthetic oligonucleotide
guaaagauuaccauagauuuaaaaa

SEQ ID NO: 999 - RNA, 25 nt, Artificial Sequence
  Description of Artificial Sequence Synthetic oligonucleotide
uguaaagauuaccauagauuuaaaa

SEQ ID NO: 1000 - RNA, 25 nt, Artificial Sequence
  Description of Artificial Sequence Synthetic oligonucleotide
uuguaaagauuaccauagauuuaaa

SEQ ID NO: 1001 - RNA, 25 nt, Artificial Sequence
  Description of Artificial Sequence Synthetic oligonucleotide
uuuguaaagauuaccauagauuuaa

SEQ ID NO: 1002 - RNA, 25 nt, Artificial Sequence
  Description of Artificial Sequence Synthetic oligonucleotide
uuuuguaaagauuaccauagauuua

SEQ ID NO: 1003 - RNA, 25 nt, Artificial Sequence
  Description of Artificial Sequence Synthetic oligonucleotide
auuuuguaaagauuaccauagauuu

SEQ ID NO: 1004 - RNA, 25 nt, Artificial Sequence
  Description of Artificial Sequence Synthetic oligonucleotide
uauuuuguaaagauuaccauagauu

SEQ ID NO: 1005 - RNA, 25 nt, Artificial Sequence
  Description of Artificial Sequence Synthetic oligonucleotide
auauuuuguaaagauuaccauagau

SEQ ID NO: 1006 - RNA, 25 nt, Artificial Sequence
  Description of Artificial Sequence Synthetic oligonucleotide
aauauuuuguaaagauuaccauaga

SEQ ID NO: 1007 - RNA, 25 nt, Artificial Sequence
  Description of Artificial Sequence Synthetic oligonucleotide
aaauauuuuguaaagauuaccauag

SEQ ID NO: 1008 - RNA, 25 nt, Artificial Sequence
  Description of Artificial Sequence Synthetic oligonucleotide
aaaauauuuuguaaagauuaccaua

SEQ ID NO: 1009 - RNA, 25 nt, Artificial Sequence
  Description of Artificial Sequence Synthetic oligonucleotide
uaaaauauuuuguaaagauuaccau

SEQ ID NO: 1010 - RNA, 25 nt, Artificial Sequence
  Description of Artificial Sequence Synthetic oligonucleotide
guaaaauauuuuguaaagauuacca

SEQ ID NO: 1011 - RNA, 25 nt, Artificial Sequence
  Description of Artificial Sequence Synthetic oligonucleotide
aguaaaauauuuuguaaagauuacc

SEQ ID NO: 1012 - RNA, 25 nt, Artificial Sequence
  Description of Artificial Sequence Synthetic oligonucleotide
aaguaaaauauuuuguaaagauuac

SEQ ID NO: 1013 - RNA, 25 nt, Artificial Sequence
  Description of Artificial Sequence Synthetic oligonucleotide
gaaguaaaauauuuuguaaagauua

SEQ ID NO: 1014 - RNA, 25 nt, Artificial Sequence
  Description of Artificial Sequence Synthetic oligonucleotide
ggaaguaaaauauuuuguaaagauu

SEQ ID NO: 1015 - RNA, 25 nt, Artificial Sequence
  Description of Artificial Sequence Synthetic oligonucleotide
cggaaguaaaauauuuuguaaagau

SEQ ID NO: 1016 - RNA, 25 nt, Artificial Sequence
  Description of Artificial Sequence Synthetic oligonucleotide
ucggaaguaaaauauuuuguaaaga

SEQ ID NO: 1017 - RNA, 25 nt, Artificial Sequence
  Description of Artificial Sequence Synthetic oligonucleotide
uucggaaguaaaauauuuuguaaag

SEQ ID NO: 1018 - RNA, 25 nt, Artificial Sequence
  Description of Artificial Sequence Synthetic oligonucleotide
guucggaaguaaaauauuuuguaaa

SEQ ID NO: 1019 - RNA, 25 nt, Artificial Sequence
  Description of Artificial Sequence Synthetic oligonucleotide
aguucggaaguaaaauauuuuguaa

SEQ ID NO: 1020 - RNA, 25 nt, Artificial Sequence
  Description of Artificial Sequence Synthetic oligonucleotide
gaguucggaaguaaaauauuuugua

SEQ ID NO: 1021 - RNA, 25 nt, Artificial Sequence
  Description of Artificial Sequence Synthetic oligonucleotide
ugaguucggaaguaaaauauuuugu

SEQ ID NO: 1022 - RNA, 25 nt, Artificial Sequence
  Description of Artificial Sequence Synthetic oligonucleotide
augaguucggaaguaaaauauuuug

SEQ ID NO: 1023 - RNA, 25 nt, Artificial Sequence
  Description of Artificial Sequence Synthetic oligonucleotide
uaugaguucggaaguaaaauauuuu

SEQ ID NO: 1024 - RNA, 25 nt, Artificial Sequence
  Description of Artificial Sequence Synthetic oligonucleotide
auaugaguucggaaguaaaauauuu

SEQ ID NO: 1025 - RNA, 25 nt, Artificial Sequence
  Description of Artificial Sequence Synthetic oligonucleotide
uauaugaguucggaaguaaaauauu

SEQ ID NO: 1026 - RNA, 25 nt, Artificial Sequence
  Description of Artificial Sequence Synthetic oligonucleotide
guauaugaguucggaaguaaaauau

SEQ ID NO: 1027 - RNA, 25 nt, Artificial Sequence
  Description of Artificial Sequence Synthetic oligonucleotide
gguauaugaguucggaaguaaaaua

SEQ ID NO: 1028 - RNA, 25 nt, Artificial Sequence
  Description of Artificial Sequence Synthetic oligonucleotide
agguauaugaguucggaaguaaaau

SEQ ID NO: 1029 - RNA, 25 nt, Artificial Sequence
  Description of Artificial Sequence Synthetic oligonucleotide
cagguauaugaguucggaaguaaaa

SEQ ID NO: 1030 - RNA, 25 nt, Artificial Sequence
  Description of Artificial Sequence Synthetic oligonucleotide
ccagguauaugaguucggaaguaaa

SEQ ID NO: 1031 - RNA, 25 nt, Artificial Sequence
  Description of Artificial Sequence Synthetic oligonucleotide
cccagguauaugaguucggaaguaa

SEQ ID NO: 1032 - RNA, 25 nt, Artificial Sequence
  Description of Artificial Sequence Synthetic oligonucleotide
ccccagguauaugaguucggaagua

SEQ ID NO: 1033 - RNA, 25 nt, Artificial Sequence
  Description of Artificial Sequence Synthetic oligonucleotide
uccccagguauaugaguucggaagu

SEQ ID NO: 1034 - RNA, 25 nt, Artificial Sequence
  Description of Artificial Sequence Synthetic oligonucleotide
auccccagguauaugaguucggaag

SEQ ID NO: 1035 - RNA, 25 nt, Artificial Sequence
  Description of Artificial Sequence Synthetic oligonucleotide
aauccccagguauaugaguucggaa

SEQ ID NO: 1036 - RNA, 25 nt, Artificial Sequence
  Description of Artificial Sequence Synthetic oligonucleotide
aaauccccagguauaugaguucgga

SEQ ID NO: 1037 - RNA, 25 nt, Artificial Sequence
  Description of Artificial Sequence Synthetic oligonucleotide
aaaauccccagguauaugaguucgg

SEQ ID NO: 1038 - RNA, 25 nt, Artificial Sequence
  Description of Artificial Sequence Synthetic oligonucleotide
uaaaauccccagguauaugaguucg

SEQ ID NO: 1039 - RNA, 25 nt, Artificial Sequence
  Description of Artificial Sequence Synthetic oligonucleotide
auaaaauccccagguauaugaguuc

SEQ ID NO: 1040 - RNA, 25 nt, Artificial Sequence
  Description of Artificial Sequence Synthetic oligonucleotide
aauaaaauccccagguauaugaguu

SEQ ID NO: 1041 - RNA, 25 nt, Artificial Sequence
  Description of Artificial Sequence Synthetic oligonucleotide
uaauaaaauccccagguauaugagu

SEQ ID NO: 1042 - RNA, 25 nt, Artificial Sequence
  Description of Artificial Sequence Synthetic oligonucleotide
guaauaaaauccccagguauaugag

SEQ ID NO: 1043 - RNA, 25 nt, Artificial Sequence
  Description of Artificial Sequence Synthetic oligonucleotide
aguaauaaaauccccagguauauga

SEQ ID NO: 1044 - RNA, 25 nt, Artificial Sequence
  Description of Artificial Sequence Synthetic oligonucleotide
gaguaauaaaauccccagguauaug

SEQ ID NO: 1045 - RNA, 25 nt, Artificial Sequence
  Description of Artificial Sequence Synthetic oligonucleotide
agaguaauaaaauccccagguauau

SEQ ID NO: 1046 - RNA, 25 nt, Artificial Sequence
  Description of Artificial Sequence Synthetic oligonucleotide
cagaguaauaaaauccccagguaua

SEQ ID NO: 1047 - RNA, 25 nt, Artificial Sequence
  Description of Artificial Sequence Synthetic oligonucleotide
ccagaguaauaaaauccccagguau

SEQ ID NO: 1048 - RNA, 25 nt, Artificial Sequence
  Description of Artificial Sequence Synthetic oligonucleotide
cccagaguaauaaaauccccaggua

SEQ ID NO: 1049 - RNA, 25 nt, Artificial Sequence
  Description of Artificial Sequence Synthetic oligonucleotide
ucccagaguaauaaaauccccaggu

SEQ ID NO: 1050 - RNA, 25 nt, Artificial Sequence
  Description of Artificial Sequence Synthetic oligonucleotide
uucccagaguaauaaaauccccagg

SEQ ID NO: 1051 - RNA, 25 nt, Artificial Sequence
  Description of Artificial Sequence Synthetic oligonucleotide
auucccagaguaauaaaauccccag

SEQ ID NO: 1052 - RNA, 25 nt, Artificial Sequence
  Description of Artificial Sequence Synthetic oligonucleotide
aauucccagaguaauaaaaucccca

SEQ ID NO: 1053 - RNA, 25 nt, Artificial Sequence
  Description of Artificial Sequence Synthetic oligonucleotide
uaauucccagaguaauaaaaucccc

SEQ ID NO: 1054 - RNA, 25 nt, Artificial Sequence
  Description of Artificial Sequence Synthetic oligonucleotide
auaauucccagaguaauaaaauccc

SEQ ID NO: 1055 - RNA, 25 nt, Artificial Sequence
  Description of Artificial Sequence Synthetic oligonucleotide
cauaauucccagaguaauaaaaucc

SEQ ID NO: 1056 - RNA, 25 nt, Artificial Sequence
  Description of Artificial Sequence Synthetic oligonucleotide
acauaauucccagaguaauaaaauc

SEQ ID NO: 1057 - RNA, 25 nt, Artificial Sequence
  Description of Artificial Sequence Synthetic oligonucleotide
cacauaauucccagaguaauaaaau

SEQ ID NO: 1058 - RNA, 25 nt, Artificial Sequence
  Description of Artificial Sequence Synthetic oligonucleotide
acacauaauucccagaguaauaaaa

SEQ ID NO: 1059 - RNA, 25 nt, Artificial Sequence
  Description of Artificial Sequence Synthetic oligonucleotide
aacacauaauucccagaguaauaaa

SEQ ID NO: 1060 - RNA, 25 nt, Artificial Sequence
  Description of Artificial Sequence Synthetic oligonucleotide
gaacacauaauucccagaguaauaa

SEQ ID NO: 1061 - RNA, 25 nt, Artificial Sequence
  Description of Artificial Sequence Synthetic oligonucleotide
agaacacauaauucccagaguaaua

SEQ ID NO: 1062 - RNA, 25 nt, Artificial Sequence
  Description of Artificial Sequence Synthetic oligonucleotide
cagaacacauaauucccagaguaau

SEQ ID NO: 1063 - RNA, 25 nt, Artificial Sequence
  Description of Artificial Sequence Synthetic oligonucleotide
gcagaacacauaauucccagaguaa

SEQ ID NO: 1064 - RNA, 25 nt, Artificial Sequence
  Description of Artificial Sequence Synthetic oligonucleotide
ggcagaacacauaauucccagagua

SEQ ID NO: 1065 - RNA, 25 nt, Artificial Sequence
  Description of Artificial Sequence Synthetic oligonucleotide
gggcagaacacauaauucccagagu

SEQ ID NO: 1066 - RNA, 25 nt, Artificial Sequence
  Description of Artificial Sequence Synthetic oligonucleotide
ggggcagaacacauaauucccagag

SEQ ID NO: 1067 - RNA, 25 nt, Artificial Sequence
  Description of Artificial Sequence Synthetic oligonucleotide
uggggcagaacacauaauucccaga

SEQ ID NO: 1068 - RNA, 25 nt, Artificial Sequence
  Description of Artificial Sequence Synthetic oligonucleotide
auggggcagaacacauaauucccag

SEQ ID NO: 1069 - RNA, 25 nt, Artificial Sequence
  Description of Artificial Sequence Synthetic oligonucleotide
gauggggcagaacacauaauuccca

SEQ ID NO: 1070 - RNA, 25 nt, Artificial Sequence
  Description of Artificial Sequence Synthetic oligonucleotide
ugauggggcagaacacauaauuccc

SEQ ID NO: 1071 - RNA, 25 nt, Artificial Sequence
  Description of Artificial Sequence Synthetic oligonucleotide
gugauggggcagaacacauaauucc

SEQ ID NO: 1072 - RNA, 25 nt, Artificial Sequence
  Description of Artificial Sequence Synthetic oligonucleotide
agugauggggcagaacacauaauuc

SEQ ID NO: 1073 - RNA, 25 nt, Artificial Sequence
  Description of Artificial Sequence Synthetic oligonucleotide
gagugauggggcagaacacauaauu

SEQ ID NO: 1074 - RNA, 25 nt, Artificial Sequence
  Description of Artificial Sequence Synthetic oligonucleotide
agagugauggggcagaacacauaau

SEQ ID NO: 1075 - RNA, 25 nt, Artificial Sequence
  Description of Artificial Sequence Synthetic oligonucleotide
gagagugauggggcagaacacauaa

SEQ ID NO: 1076 - RNA, 25 nt, Artificial Sequence
  Description of Artificial Sequence Synthetic oligonucleotide
agagagugauggggcagaacacaua

SEQ ID NO: 1077 - RNA, 25 nt, Artificial Sequence
  Description of Artificial Sequence Synthetic oligonucleotide
gagagagugauggggcagaacacau

SEQ ID NO: 1078 - RNA, 25 nt, Artificial Sequence
  Description of Artificial Sequence Synthetic oligonucleotide
agagagagugauggggcagaacaca

SEQ ID NO: 1079 - RNA, 25 nt, Artificial Sequence
  Description of Artificial Sequence Synthetic oligonucleotide
aagagagagugauggggcagaacac

SEQ ID NO: 1080 - RNA, 25 nt, Artificial Sequence
  Description of Artificial Sequence Synthetic oligonucleotide
uaagagagagugauggggcagaaca

SEQ ID NO: 1081 - RNA, 25 nt, Artificial Sequence
  Description of Artificial Sequence Synthetic oligonucleotide
uuaagagagagugauggggcagaac

SEQ ID NO: 1082 - RNA, 25 nt, Artificial Sequence
  Description of Artificial Sequence Synthetic oligonucleotide
auuaagagagagugauggggcagaa

SEQ ID NO: 1083 - RNA, 25 nt, Artificial Sequence
  Description of Artificial Sequence Synthetic oligonucleotide
aauuaagagagagugauggggcaga

SEQ ID NO: 1084 - RNA, 25 nt, Artificial Sequence
  Description of Artificial Sequence Synthetic oligonucleotide
caauuaagagagagugauggggcag

SEQ ID NO: 1085 - RNA, 25 nt, Artificial Sequence
  Description of Artificial Sequence Synthetic oligonucleotide
ccaauuaagagagagugauggggca

SEQ ID NO: 1086 - RNA, 25 nt, Artificial Sequence
  Description of Artificial Sequence Synthetic oligonucleotide
uccaauuaagagagagugauggggc

SEQ ID NO: 1087 - RNA, 25 nt, Artificial Sequence
  Description of Artificial Sequence Synthetic oligonucleotide
auccaauuaagagagagugaugggg

SEQ ID NO: 1088 - RNA, 25 nt, Artificial Sequence
  Description of Artificial Sequence Synthetic oligonucleotide
aauccaauuaagagagagugauggg

SEQ ID NO: 1089 - RNA, 25 nt, Artificial Sequence
  Description of Artificial Sequence Synthetic oligonucleotide
aaauccaauuaagagagagugaugg

SEQ ID NO: 1090 - RNA, 25 nt, Artificial Sequence
  Description of Artificial Sequence Synthetic oligonucleotide
aaaauccaauuaagagagagugaug

SEQ ID NO: 1091 - RNA, 25 nt, Artificial Sequence
  Description of Artificial Sequence Synthetic oligonucleotide
aaaaauccaauuaagagagagugau

SEQ ID NO: 1092 - RNA, 25 nt, Artificial Sequence
  Description of Artificial Sequence Synthetic oligonucleotide
uaaaaauccaauuaagagagaguga

SEQ ID NO: 1093 - RNA, 25 nt, Artificial Sequence
  Description of Artificial Sequence Synthetic oligonucleotide
uuaaaaauccaauuaagagagagug

SEQ ID NO: 1094 - RNA, 25 nt, Artificial Sequence
  Description of Artificial Sequence Synthetic oligonucleotide
uuuaaaaauccaauuaagagagagu

SEQ ID NO: 1095 - RNA, 25 nt, Artificial Sequence
  Description of Artificial Sequence Synthetic oligonucleotide
uuuuaaaaauccaauuaagagagag

SEQ ID NO: 1096 - RNA, 25 nt, Artificial Sequence
  Description of Artificial Sequence Synthetic oligonucleotide
auuuuaaaaauccaauuaagagaga

SEQ ID NO: 1097 - RNA, 25 nt, Artificial Sequence
  Description of Artificial Sequence Synthetic oligonucleotide
aauuuuaaaaauccaauuaagagag

SEQ ID NO: 1098 - RNA, 25 nt, Artificial Sequence
  Description of Artificial Sequence Synthetic oligonucleotide
uaauuuuaaaaauccaauuaagaga

SEQ ID NO: 1099 - RNA, 25 nt, Artificial Sequence
  Description of Artificial Sequence Synthetic oligonucleotide
auaauuuuaaaaauccaauuaagag

SEQ ID NO: 1100 - RNA, 25 nt, Artificial Sequence
  Description of Artificial Sequence Synthetic oligonucleotide
uauaauuuuaaaaauccaauuaaga

SEQ ID NO: 1101 - RNA, 25 nt, Artificial Sequence
  Description of Artificial Sequence Synthetic oligonucleotide
auauaauuuuaaaaauccaauuaag

SEQ ID NO: 1102 - RNA, 25 nt, Artificial Sequence
  Description of Artificial Sequence Synthetic oligonucleotide
aauauaauuuuaaaaauccaauuaa

SEQ ID NO: 1103 - RNA, 25 nt, Artificial Sequence
  Description of Artificial Sequence Synthetic oligonucleotide
gaauauaauuuuaaaaauccaauua

SEQ ID NO: 1104 - RNA, 25 nt, Artificial Sequence
  Description of Artificial Sequence Synthetic oligonucleotide
ugaauauaauuuuaaaaauccaauu

SEQ ID NO: 1105 - RNA, 25 nt, Artificial Sequence
  Description of Artificial Sequence Synthetic oligonucleotide
augaauauaauuuuaaaaauccaau

SEQ ID NO: 1106 - RNA, 25 nt, Artificial Sequence
  Description of Artificial Sequence Synthetic oligonucleotide
uaugaauauaauuuuaaaaauccaa

SEQ ID NO: 1107 - RNA, 25 nt, Artificial Sequence
  Description of Artificial Sequence Synthetic oligonucleotide
auaugaauauaauuuuaaaaaucca

SEQ ID NO: 1108 - RNA, 25 nt, Artificial Sequence
  Description of Artificial Sequence Synthetic oligonucleotide
aauaugaauauaauuuuaaaaaucc

SEQ ID NO: 1109 - RNA, 25 nt, Artificial Sequence
  Description of Artificial Sequence Synthetic oligonucleotide
caauaugaauauaauuuuaaaaauc

SEQ ID NO: 1110 - RNA, 25 nt, Artificial Sequence
  Description of Artificial Sequence Synthetic oligonucleotide
gcaauaugaauauaauuuuaaaaau

SEQ ID NO: 1111 - RNA, 25 nt, Artificial Sequence
  Description of Artificial Sequence Synthetic oligonucleotide
ugcaauaugaauauaauuuuaaaaa

SEQ ID NO: 1112 - RNA, 25 nt, Artificial Sequence
  Description of Artificial Sequence Synthetic oligonucleotide
cugcaauaugaauauaauuuuaaaa

SEQ ID NO: 1113 - RNA, 25 nt, Artificial Sequence
  Description of Artificial Sequence Synthetic oligonucleotide
ccugcaauaugaauauaauuuuaaa

SEQ ID NO: 1114 - RNA, 25 nt, Artificial Sequence
  Description of Artificial Sequence Synthetic oligonucleotide
uccugcaauaugaauauaauuuuaa

SEQ ID NO: 1115 - RNA, 25 nt, Artificial Sequence
  Description of Artificial Sequence Synthetic oligonucleotide
guccugcaauaugaauauaauuuua

SEQ ID NO: 1116 - RNA, 25 nt, Artificial Sequence
  Description of Artificial Sequence Synthetic oligonucleotide
aguccugcaauaugaauauaauuuu

SEQ ID NO: 1117 - RNA, 25 nt, Artificial Sequence
  Description of Artificial Sequence Synthetic oligonucleotide
gaguccugcaauaugaauauaauuu

SEQ ID NO: 1118 - RNA, 25 nt, Artificial Sequence
  Description of Artificial Sequence Synthetic oligonucleotide
cgaguccugcaauaugaauauaauu

SEQ ID NO: 1119 - RNA, 25 nt, Artificial Sequence
  Description of Artificial Sequence Synthetic oligonucleotide
ccgaguccugcaauaugaauauaau

SEQ ID NO: 1120 - RNA, 25 nt, Artificial Sequence
  Description of Artificial Sequence Synthetic oligonucleotide
gccgaguccugcaauaugaauauaa

SEQ ID NO: 1121 - RNA, 25 nt, Artificial Sequence
  Description of Artificial Sequence Synthetic oligonucleotide
ugccgaguccugcaauaugaauaua

SEQ ID NO: 1122 - RNA, 25 nt, Artificial Sequence
  Description of Artificial Sequence Synthetic oligonucleotide
cugccgaguccugcaauaugaauau

SEQ ID NO: 1123 - RNA, 25 nt, Artificial Sequence
  Description of Artificial Sequence Synthetic oligonucleotide
ucugccgaguccugcaauaugaaua

SEQ ID NO: 1124 - RNA, 25 nt, Artificial Sequence
  Description of Artificial Sequence Synthetic oligonucleotide
uucugccgaguccugcaauaugaau

SEQ ID NO: 1125 - RNA, 25 nt, Artificial Sequence
  Description of Artificial Sequence Synthetic oligonucleotide
cuucugccgaguccugcaauaugaa

SEQ ID NO: 1126 - RNA, 25 nt, Artificial Sequence
  Description of Artificial Sequence Synthetic oligonucleotide
ucuucugccgaguccugcaauauga

SEQ ID NO: 1127 - RNA, 25 nt, Artificial Sequence
  Description of Artificial Sequence Synthetic oligonucleotide
gucuucugccgaguccugcaauaug

SEQ ID NO: 1128 - RNA, 25 nt, Artificial Sequence
  Description of Artificial Sequence Synthetic oligonucleotide
ggucuucugccgaguccugcaauau

SEQ ID NO: 1129 - RNA, 25 nt, Artificial Sequence
  Description of Artificial Sequence Synthetic oligonucleotide
aggucuucugccgaguccugcaaua

SEQ ID NO: 1130 - RNA, 25 nt, Artificial Sequence
  Description of Artificial Sequence Synthetic oligonucleotide
aaggucuucugccgaguccugcaau

SEQ ID NO: 1131 - RNA, 25 nt, Artificial Sequence
  Description of Artificial Sequence Synthetic oligonucleotide
gaaggucuucugccgaguccugcaa

SEQ ID NO: 1132 - RNA, 25 nt, Artificial Sequence
  Description of Artificial Sequence Synthetic oligonucleotide
cgaaggucuucugccgaguccugca

SEQ ID NO: 1133 - RNA, 25 nt, Artificial Sequence
  Description of Artificial Sequence Synthetic oligonucleotide
ucgaaggucuucugccgaguccugc

SEQ ID NO: 1134 - RNA, 25 nt, Artificial Sequence
  Description of Artificial Sequence Synthetic oligonucleotide
cucgaaggucuucugccgaguccug

SEQ ID NO: 1135 - RNA, 25 nt, Artificial Sequence
  Description of Artificial Sequence Synthetic oligonucleotide
ucucgaaggucuucugccgaguccu

SEQ ID NO: 1136 - RNA, 25 nt, Artificial Sequence
  Description of Artificial Sequence Synthetic oligonucleotide
cucucgaaggucuucugccgagucc

SEQ ID NO: 1137 - RNA, 25 nt, Artificial Sequence
  Description of Artificial Sequence Synthetic oligonucleotide
ucucucgaaggucuucugccgaguc

SEQ ID NO: 1138 - RNA, 25 nt, Artificial Sequence
  Description of Artificial Sequence Synthetic oligonucleotide
uucucucgaaggucuucugccgagu

SEQ ID NO: 1139 - RNA, 25 nt, Artificial Sequence
  Description of Artificial Sequence Synthetic oligonucleotide
uuucucucgaaggucuucugccgag

SEQ ID NO: 1140 - RNA, 25 nt, Artificial Sequence
  Description of Artificial Sequence Synthetic oligonucleotide
cuuucucucgaaggucuucugccga

SEQ ID NO: 1141 - RNA, 25 nt, Artificial Sequence
  Description of Artificial Sequence Synthetic oligonucleotide
ccuuucucucgaaggucuucugccg

SEQ ID NO: 1142 - RNA, 25 nt, Artificial Sequence
  Description of Artificial Sequence Synthetic oligonucleotide
accuuucucucgaaggucuucugcc

SEQ ID NO: 1143 - RNA, 25 nt, Artificial Sequence
  Description of Artificial Sequence Synthetic oligonucleotide
uaccuuucucucgaaggucuucugc

SEQ ID NO: 1144 - RNA, 25 nt, Artificial Sequence
  Description of Artificial Sequence Synthetic oligonucleotide
cuaccuuucucucgaaggucuucug

SEQ ID NO: 1145 - RNA, 25 nt, Artificial Sequence
  Description of Artificial Sequence Synthetic oligonucleotide
ucuaccuuucucucgaaggucuucu

SEQ ID NO: 1146 - RNA, 25 nt, Artificial Sequence
  Description of Artificial Sequence Synthetic oligonucleotide
uucuaccuuucucucgaaggucuuc

SEQ ID NO: 1147 - RNA, 25 nt, Artificial Sequence
  Description of Artificial Sequence Synthetic oligonucleotide
uuucuaccuuucucucgaaggucuu

SEQ ID NO: 1148 - RNA, 25 nt, Artificial Sequence
  Description of Artificial Sequence Synthetic oligonucleotide
uuuucuaccuuucucucgaaggucu

SEQ ID NO: 1149 - RNA, 25 nt, Artificial Sequence
  Description of Artificial Sequence Synthetic oligonucleotide
auuuucuaccuuucucucgaagguc

SEQ ID NO: 1150 - RNA, 25 nt, Artificial Sequence
  Description of Artificial Sequence Synthetic oligonucleotide
uauuuucuaccuuucucucgaaggu

SEQ ID NO: 1151 - RNA, 25 nt, Artificial Sequence
  Description of Artificial Sequence Synthetic oligonucleotide
uuauuuucuaccuuucucucgaagg

SEQ ID NO: 1152 - RNA, 25 nt, Artificial Sequence
  Description of Artificial Sequence Synthetic oligonucleotide
cuuauuuucuaccuuucucucgaag

SEQ ID NO: 1153 - RNA, 25 nt, Artificial Sequence
  Description of Artificial Sequence Synthetic oligonucleotide
ucuuauuuucuaccuuucucucgaa

SEQ ID NO: 1154 - RNA, 25 nt, Artificial Sequence
  Description of Artificial Sequence Synthetic oligonucleotide
uucuuauuuucuaccuuucucucga

SEQ ID NO: 1155 - RNA, 25 nt, Artificial Sequence
  Description of Artificial Sequence Synthetic oligonucleotide
auucuuauuuucuaccuuucucucg

SEQ ID NO: 1156 - RNA, 25 nt, Artificial Sequence
  Description of Artificial Sequence Synthetic oligonucleotide
aauucuuauuuucuaccuuucucuc

SEQ ID NO: 1157 - RNA, 25 nt, Artificial Sequence
  Description of Artificial Sequence Synthetic oligonucleotide
aaauucuuauuuucuaccuuucucu

SEQ ID NO: 1158 - RNA, 25 nt, Artificial Sequence
  Description of Artificial Sequence Synthetic oligonucleotide
caaauucuuauuuucuaccuuucuc

SEQ ID NO: 1159 - RNA, 25 nt, Artificial Sequence
  Description of Artificial Sequence Synthetic oligonucleotide
ccaaauucuuauuuucuaccuuucu

SEQ ID NO: 1160 - RNA, 25 nt, Artificial Sequence
  Description of Artificial Sequence Synthetic oligonucleotide
gccaaauucuuauuuucuaccuuuc

SEQ ID NO: 1161 - RNA, 25 nt, Artificial Sequence
  Description of Artificial Sequence Synthetic oligonucleotide
agccaaauucuuauuuucuaccuuu

SEQ ID NO: 1162 - RNA, 25 nt, Artificial Sequence
  Description of Artificial Sequence Synthetic oligonucleotide
gagccaaauucuuauuuucuaccuu

SEQ ID NO: 1163 - RNA, 25 nt, Artificial Sequence
  Description of Artificial Sequence Synthetic oligonucleotide
agagccaaauucuuauuuucuaccu

SEQ ID NO: 1164 - RNA, 25 nt, Artificial Sequence
  Description of Artificial Sequence Synthetic oligonucleotide
gagagccaaauucuuauuuucuacc

SEQ ID NO: 1165 - RNA, 25 nt, Artificial Sequence
  Description of Artificial Sequence Synthetic oligonucleotide
agagagccaaauucuuauuuucuac

SEQ ID NO: 1166 - RNA, 25 nt, Artificial Sequence
  Description of Artificial Sequence Synthetic oligonucleotide
cagagagccaaauucuuauuuucua

SEQ ID NO: 1167 - RNA, 25 nt, Artificial Sequence
  Description of Artificial Sequence Synthetic oligonucleotide
acagagagccaaauucuuauuuucu

SEQ ID NO: 1168 - RNA, 25 nt, Artificial Sequence
  Description of Artificial Sequence Synthetic oligonucleotide
cacagagagccaaauucuuauuuuc

SEQ ID NO: 1169 - RNA, 25 nt, Artificial Sequence
  Description of Artificial Sequence Synthetic oligonucleotide
acacagagagccaaauucuuauuuu

SEQ ID NO: 1170 - RNA, 25 nt, Artificial Sequence
  Description of Artificial Sequence Synthetic oligonucleotide
cacacagagagccaaauucuuauuu

SEQ ID NO: 1171 - RNA, 25 nt, Artificial Sequence
  Description of Artificial Sequence Synthetic oligonucleotide
ucacacagagagccaaauucuuauu

SEQ ID NO: 1172 - RNA, 25 nt, Artificial Sequence
  Description of Artificial Sequence Synthetic oligonucleotide
cucacacagagagccaaauucuuau

SEQ ID NO: 1173 - RNA, 25 nt, Artificial Sequence
  Description of Artificial Sequence Synthetic oligonucleotide
gcucacacagagagccaaauucuua

SEQ ID NO: 1174 - RNA, 25 nt, Artificial Sequence
  Description of Artificial Sequence Synthetic oligonucleotide
ugcucacacagagagccaaauucuu

SEQ ID NO: 1175 - RNA, 25 nt, Artificial Sequence
  Description of Artificial Sequence Synthetic oligonucleotide
augcucacacagagagccaaauucu

SEQ ID NO: 1176 - RNA, 25 nt, Artificial Sequence
  Description of Artificial Sequence Synthetic oligonucleotide
caugcucacacagagagccaaauuc

SEQ ID NO: 1177 - RNA, 25 nt, Artificial Sequence
  Description of Artificial Sequence Synthetic oligonucleotide
acaugcucacacagagagccaaauu

SEQ ID NO: 1178 - RNA, 25 nt, Artificial Sequence
  Description of Artificial Sequence Synthetic oligonucleotide
cacaugcucacacagagagccaaau

SEQ ID NO: 1179 - RNA, 25 nt, Artificial Sequence
  Description of Artificial Sequence Synthetic oligonucleotide
acacaugcucacacagagagccaaa

SEQ ID NO: 1180 - RNA, 25 nt, Artificial Sequence
  Description of Artificial Sequence Synthetic oligonucleotide
cacacaugcucacacagagagccaa

SEQ ID NO: 1181 - RNA, 25 nt, Artificial Sequence
  Description of Artificial Sequence Synthetic oligonucleotide
gcacacaugcucacacagagagcca

SEQ ID NO: 1182 - RNA, 25 nt, Artificial Sequence
  Description of Artificial Sequence Synthetic oligonucleotide
cgcacacaugcucacacagagagcc

SEQ ID NO: 1183 - RNA, 25 nt, Artificial Sequence
  Description of Artificial Sequence Synthetic oligonucleotide
acgcacacaugcucacacagagagc

SEQ ID NO: 1184 - RNA, 25 nt, Artificial Sequence
  Description of Artificial Sequence Synthetic oligonucleotide
cacgcacacaugcucacacagagag

SEQ ID NO: 1185 - RNA, 25 nt, Artificial Sequence
  Description of Artificial Sequence Synthetic oligonucleotide
acacgcacacaugcucacacagaga

SEQ ID NO: 1186 - RNA, 25 nt, Artificial Sequence
  Description of Artificial Sequence Synthetic oligonucleotide
cacacgcacacaugcucacacagag

SEQ ID NO: 1187 - RNA, 25 nt, Artificial Sequence
  Description of Artificial Sequence Synthetic oligonucleotide
acacacgcacacaugcucacacaga

SEQ ID NO: 1188 - RNA, 25 nt, Artificial Sequence
  Description of Artificial Sequence Synthetic oligonucleotide
cacacacgcacacaugcucacacag

SEQ ID NO: 1189 - RNA, 25 nt, Artificial Sequence
  Description of Artificial Sequence Synthetic oligonucleotide
gcacacacgcacacaugcucacaca

SEQ ID NO: 1190 - RNA, 25 nt, Artificial Sequence
  Description of Artificial Sequence Synthetic oligonucleotide
cgcacacacgcacacaugcucacac

SEQ ID NO: 1191 - RNA, 25 nt, Artificial Sequence
  Description of Artificial Sequence Synthetic oligonucleotide
ucgcacacacgcacacaugcucaca

SEQ ID NO: 1192 - RNA, 25 nt, Artificial Sequence
  Description of Artificial Sequence Synthetic oligonucleotide
cucgcacacacgcacacaugcucac

SEQ ID NO: 1193 - RNA, 25 nt, Artificial Sequence
  Description of Artificial Sequence Synthetic oligonucleotide
ucucgcacacacgcacacaugcuca

SEQ ID NO: 1194 - RNA, 25 nt, Artificial Sequence
  Description of Artificial Sequence Synthetic oligonucleotide
cucucgcacacacgcacacaugcuc

SEQ ID NO: 1195 - RNA, 25 nt, Artificial Sequence
  Description of Artificial Sequence Synthetic oligonucleotide
ucucucgcacacacgcacacaugcu

SEQ ID NO: 1196 - RNA, 25 nt, Artificial Sequence
  Description of Artificial Sequence Synthetic oligonucleotide
cucucucgcacacacgcacacaugc

SEQ ID NO: 1197 - RNA, 25 nt, Artificial Sequence
  Description of Artificial Sequence Synthetic oligonucleotide
ucucucucgcacacacgcacacaug

SEQ ID NO: 1198 - RNA, 25 nt, Artificial Sequence
  Description of Artificial Sequence Synthetic oligonucleotide
cucucucucgcacacacgcacacau

SEQ ID NO: 1199 - RNA, 25 nt, Artificial Sequence
  Description of Artificial Sequence Synthetic oligonucleotide
ucucucucucgcacacacgcacaca

SEQ ID NO: 1200 - RNA, 25 nt, Artificial Sequence
  Description of Artificial Sequence Synthetic oligonucleotide
cucucucucucgcacacacgcacac

SEQ ID NO: 1201 - RNA, 25 nt, Artificial Sequence
  Description of Artificial Sequence Synthetic oligonucleotide
ucucucucucucgcacacacgcaca

SEQ ID NO: 1202 - RNA, 25 nt, Artificial Sequence
  Description of Artificial Sequence Synthetic oligonucleotide
gucucucucucucgcacacacgcac

SEQ ID NO: 1203 - RNA, 25 nt, Artificial Sequence
  Description of Artificial Sequence Synthetic oligonucleotide
ugucucucucucucgcacacacgca

SEQ ID NO: 1204 - RNA, 25 nt, Artificial Sequence
  Description of Artificial Sequence Synthetic oligonucleotide
cugucucucucucucgcacacacgc

SEQ ID NO: 1205 - RNA, 25 nt, Artificial Sequence
  Description of Artificial Sequence Synthetic oligonucleotide
ucugucucucucucucgcacacacg

SEQ ID NO: 1206 - RNA, 25 nt, Artificial Sequence
  Description of Artificial Sequence Synthetic oligonucleotide
gucugucucucucucucgcacacac

SEQ ID NO: 1207 - RNA, 25 nt, Artificial Sequence
  Description of Artificial Sequence Synthetic oligonucleotide
ugucugucucucucucucgcacaca

SEQ ID NO: 1208 - RNA, 25 nt, Artificial Sequence
  Description of Artificial Sequence Synthetic oligonucleotide
cugucugucucucucucucgcacac

SEQ ID NO: 1209 - RNA, 25 nt, Artificial Sequence
  Description of Artificial Sequence Synthetic oligonucleotide
gcugucugucucucucucucgcaca

SEQ ID NO: 1210 - RNA, 25 nt, Artificial Sequence
  Description of Artificial Sequence Synthetic oligonucleotide
ggcugucugucucucucucucgcac

SEQ ID NO: 1211 - RNA, 25 nt, Artificial Sequence
  Description of Artificial Sequence Synthetic oligonucleotide
aggcugucugucucucucucucgca

SEQ ID NO: 1212 - RNA, 25 nt, Artificial Sequence
  Description of Artificial Sequence Synthetic oligonucleotide
caggcugucugucucucucucucgc

SEQ ID NO: 1213 - RNA, 25 nt, Artificial Sequence
  Description of Artificial Sequence Synthetic oligonucleotide
gcaggcugucugucucucucucucg

SEQ ID NO: 1214 - RNA, 25 nt, Artificial Sequence
  Description of Artificial Sequence Synthetic oligonucleotide
ggcaggcugucugucucucucucuc

SEQ ID NO: 1215 - RNA, 25 nt, Artificial Sequence
  Description of Artificial Sequence Synthetic oligonucleotide
aggcaggcugucugucucucucucu

SEQ ID NO: 1216 - RNA, 25 nt, Artificial Sequence
  Description of Artificial Sequence Synthetic oligonucleotide
uaggcaggcugucugucucucucuc

SEQ ID NO: 1217 - RNA, 25 nt, Artificial Sequence
  Description of Artificial Sequence Synthetic oligonucleotide
uuaggcaggcugucugucucucucu

SEQ ID NO: 1218 - RNA, 25 nt, Artificial Sequence
  Description of Artificial Sequence Synthetic oligonucleotide
cuuaggcaggcugucugucucucuc

SEQ ID NO: 1219 - RNA, 25 nt, Artificial Sequence
  Description of Artificial Sequence Synthetic oligonucleotide
ucuuaggcaggcugucugucucucu

SEQ ID NO: 1220 - RNA, 25 nt, Artificial Sequence
  Description of Artificial Sequence Synthetic oligonucleotide
uucuuaggcaggcugucugucucuc

SEQ ID NO: 1221 - RNA, 25 nt, Artificial Sequence
  Description of Artificial Sequence Synthetic oligonucleotide
cuucuuaggcaggcugucugucucu

SEQ ID NO: 1222 - RNA, 25 nt, Artificial Sequence
  Description of Artificial Sequence Synthetic oligonucleotide
ucuucuuaggcaggcugucugucuc

SEQ ID NO: 1223 - RNA, 25 nt, Artificial Sequence
  Description of Artificial Sequence Synthetic oligonucleotide
uucuucuuaggcaggcugucugucu

SEQ ID NO: 1224 - RNA, 25 nt, Artificial Sequence
  Description of Artificial Sequence Synthetic oligonucleotide
uuucuucuuaggcaggcugucuguc

SEQ ID NO: 1225 - RNA, 25 nt, Artificial Sequence
  Description of Artificial Sequence Synthetic oligonucleotide
auuucuucuuaggcaggcugucugu

SEQ ID NO: 1226 - RNA, 25 nt, Artificial Sequence
  Description of Artificial Sequence Synthetic oligonucleotide
cauuucuucuuaggcaggcugucug

SEQ ID NO: 1227 - RNA, 25 nt, Artificial Sequence
  Description of Artificial Sequence Synthetic oligonucleotide
ucauuucuucuuaggcaggcugucu

SEQ ID NO: 1228 - RNA, 25 nt, Artificial Sequence
  Description of Artificial Sequence Synthetic oligonucleotide
uucauuucuucuuaggcaggcuguc

SEQ ID NO: 1229 - RNA, 25 nt, Artificial Sequence
  Description of Artificial Sequence Synthetic oligonucleotide
auucauuucuucuuaggcaggcugu

SEQ ID NO: 1230 - RNA, 25 nt, Artificial Sequence
  Description of Artificial Sequence Synthetic oligonucleotide
cauucauuucuucuuaggcaggcug

SEQ ID NO: 1231 - RNA, 25 nt, Artificial Sequence
  Description of Artificial Sequence Synthetic oligonucleotide
acauucauuucuucuuaggcaggcu

SEQ ID NO: 1232 - RNA, 25 nt, Artificial Sequence
  Description of Artificial Sequence Synthetic oligonucleotide
cacauucauuucuucuuaggcaggc

SEQ ID NO: 1233 - RNA, 25 nt, Artificial Sequence
  Description of Artificial Sequence Synthetic oligonucleotide
ucacauucauuucuucuuaggcagg

SEQ ID NO: 1234 - RNA, 25 nt, Artificial Sequence
  Description of Artificial Sequence Synthetic oligonucleotide
uucacauucauuucuucuuaggcag

SEQ ID NO: 1235 - RNA, 25 nt, Artificial Sequence
  Description of Artificial Sequence Synthetic oligonucleotide
auucacauucauuucuucuuaggca

SEQ ID NO: 1236 - RNA, 25 nt, Artificial Sequence
  Description of Artificial Sequence Synthetic oligonucleotide
cauucacauucauuucuucuuaggc

SEQ ID NO: 1237 - RNA, 25 nt, Artificial Sequence
  Description of Artificial Sequence Synthetic oligonucleotide
gcauucacauucauuucuucuuagg

SEQ ID NO: 1238 - RNA, 25 nt, Artificial Sequence
  Description of Artificial Sequence Synthetic oligonucleotide
cgcauucacauucauuucuucuuag

SEQ ID NO: 1239 - RNA, 25 nt, Artificial Sequence
  Description of Artificial Sequence Synthetic oligonucleotide
ccgcauucacauucauuucuucuua

SEQ ID NO: 1240 - RNA, 25 nt, Artificial Sequence
  Description of Artificial Sequence Synthetic oligonucleotide
gccgcauucacauucauuucuucuu

SEQ ID NO: 1241 - RNA, 25 nt, Artificial Sequence
  Description of Artificial Sequence Synthetic oligonucleotide
agccgcauucacauucauuucuucu

SEQ ID NO: 1242 - RNA, 25 nt, Artificial Sequence
  Description of Artificial Sequence Synthetic oligonucleotide
aagccgcauucacauucauuucuuc

SEQ ID NO: 1243 - RNA, 25 nt, Artificial Sequence
  Description of Artificial Sequence Synthetic oligonucleotide
caagccgcauucacauucauuucuu

SEQ ID NO: 1244 - RNA, 25 nt, Artificial Sequence
  Description of Artificial Sequence Synthetic oligonucleotide
acaagccgcauucacauucauuucu

SEQ ID NO: 1245 - RNA, 25 nt, Artificial Sequence
  Description of Artificial Sequence Synthetic oligonucleotide
cacaagccgcauucacauucauuuc

SEQ ID NO: 1246 - RNA, 25 nt, Artificial Sequence
  Description of Artificial Sequence Synthetic oligonucleotide
ccacaagccgcauucacauucauuu

SEQ ID NO: 1247 - RNA, 25 nt, Artificial Sequence
  Description of Artificial Sequence Synthetic oligonucleotide
gccacaagccgcauucacauucauu

SEQ ID NO: 1248 - RNA, 25 nt, Artificial Sequence
  Description of Artificial Sequence Synthetic oligonucleotide
ugccacaagccgcauucacauucau

SEQ ID NO: 1249 - RNA, 25 nt, Artificial Sequence
  Description of Artificial Sequence Synthetic oligonucleotide
gugccacaagccgcauucacauuca

SEQ ID NO: 1250 - RNA, 25 nt, Artificial Sequence
  Description of Artificial Sequence Synthetic oligonucleotide
ugugccacaagccgcauucacauuc

SEQ ID NO: 1251 - RNA, 25 nt, Artificial Sequence
  Description of Artificial Sequence Synthetic oligonucleotide
cugugccacaagccgcauucacauu

SEQ ID NO: 1252 - RNA, 25 nt, Artificial Sequence
  Description of Artificial Sequence Synthetic oligonucleotide
acugugccacaagccgcauucacau

SEQ ID NO: 1253 - RNA, 25 nt, Artificial Sequence
  Description of Artificial Sequence Synthetic oligonucleotide
aacugugccacaagccgcauucaca

SEQ ID NO: 1254 - RNA, 25 nt, Artificial Sequence
  Description of Artificial Sequence Synthetic oligonucleotide
caacugugccacaagccgcauucac

SEQ ID NO: 1255 - RNA, 25 nt, Artificial Sequence
  Description of Artificial Sequence Synthetic oligonucleotide
ucaacugugccacaagccgcauuca

SEQ ID NO: 1256 - RNA, 25 nt, Artificial Sequence
  Description of Artificial Sequence Synthetic oligonucleotide
gucaacugugccacaagccgcauuc

SEQ ID NO: 1257 - RNA, 25 nt, Artificial Sequence
  Description of Artificial Sequence Synthetic oligonucleotide
ugucaacugugccacaagccgcauu

SEQ ID NO: 1258 - RNA, 25 nt, Artificial Sequence
  Description of Artificial Sequence Synthetic oligonucleotide
uugucaacugugccacaagccgcau

SEQ ID NO: 1259 - RNA, 25 nt, Artificial Sequence
  Description of Artificial Sequence Synthetic oligonucleotide
cuugucaacugugccacaagccgca

SEQ ID NO: 1260 - RNA, 25 nt, Artificial Sequence
  Description of Artificial Sequence Synthetic oligonucleotide
ccuugucaacugugccacaagccgc

SEQ ID NO: 1261 - RNA, 25 nt, Artificial Sequence
  Description of Artificial Sequence Synthetic oligonucleotide
uccuugucaacugugccacaagccg

SEQ ID NO: 1262 - RNA, 25 nt, Artificial Sequence
  Description of Artificial Sequence Synthetic oligonucleotide
auccuugucaacugugccacaagcc

SEQ ID NO: 1263 - RNA, 25 nt, Artificial Sequence
  Description of Artificial Sequence Synthetic oligonucleotide
cauccuugucaacugugccacaagc

SEQ ID NO: 1264 - RNA, 25 nt, Artificial Sequence
  Description of Artificial Sequence Synthetic oligonucleotide
ucauccuugucaacugugccacaag

SEQ ID NO: 1265 - RNA, 25 nt, Artificial Sequence
  Description of Artificial Sequence Synthetic oligonucleotide
aucauccuugucaacugugccacaa

SEQ ID NO: 1266 - RNA, 25 nt, Artificial Sequence
  Description of Artificial Sequence Synthetic oligonucleotide
uaucauccuugucaacugugccaca

SEQ ID NO: 1267 - RNA, 25 nt, Artificial Sequence
  Description of Artificial Sequence Synthetic oligonucleotide
uuaucauccuugucaacugugccac

SEQ ID NO: 1268 - RNA, 25 nt, Artificial Sequence
  Description of Artificial Sequence Synthetic oligonucleotide
uuuaucauccuugucaacugugcca

SEQ ID NO: 1269 - RNA, 25 nt, Artificial Sequence
  Description of Artificial Sequence Synthetic oligonucleotide
auuuaucauccuugucaacugugcc

SEQ ID NO: 1270 - RNA, 25 nt, Artificial Sequence
  Description of Artificial Sequence Synthetic oligonucleotide
gauuuaucauccuugucaacugugc

SEQ ID NO: 1271 - RNA, 25 nt, Artificial Sequence
  Description of Artificial Sequence Synthetic oligonucleotide
ugauuuaucauccuugucaacugug

SEQ ID NO: 1272 - RNA, 25 nt, Artificial Sequence
  Description of Artificial Sequence Synthetic oligonucleotide
uugauuuaucauccuugucaacugu

SEQ ID NO: 1273 - RNA, 25 nt, Artificial Sequence
  Description of Artificial Sequence Synthetic oligonucleotide
auugauuuaucauccuugucaacug

SEQ ID NO: 1274 - RNA, 25 nt, Artificial Sequence
  Description of Artificial Sequence Synthetic oligonucleotide
uauugauuuaucauccuugucaacu

SEQ ID NO: 1275 - RNA, 25 nt, Artificial Sequence
  Description of Artificial Sequence Synthetic oligonucleotide
uuauugauuuaucauccuugucaac

SEQ ID NO: 1276 - RNA, 25 nt, Artificial Sequence
  Description of Artificial Sequence Synthetic oligonucleotide
auuauugauuuaucauccuugucaa

SEQ ID NO: 1277 - RNA, 25 nt, Artificial Sequence
  Description of Artificial Sequence Synthetic oligonucleotide
cauuauugauuuaucauccuuguca

SEQ ID NO: 1278 - RNA, 25 nt, Artificial Sequence
  Description of Artificial Sequence Synthetic oligonucleotide
gcauuauugauuuaucauccuuguc

SEQ ID NO: 1279 - RNA, 25 nt, Artificial Sequence
  Description of Artificial Sequence Synthetic oligonucleotide
ugcauuauugauuuaucauccuugu

SEQ ID NO: 1280 - RNA, 25 nt, Artificial Sequence
  Description of Artificial Sequence Synthetic oligonucleotide
uugcauuauugauuuaucauccuug

SEQ ID NO: 1281 - RNA, 25 nt, Artificial Sequence
  Description of Artificial Sequence Synthetic oligonucleotide
cuugcauuauugauuuaucauccuu

SEQ ID NO: 1282 - RNA, 25 nt, Artificial Sequence
  Description of Artificial Sequence Synthetic oligonucleotide
gcuugcauuauugauuuaucauccu

SEQ ID NO: 1283 - RNA, 25 nt, Artificial Sequence
  Description of Artificial Sequence Synthetic oligonucleotide
agcuugcauuauugauuuaucaucc

SEQ ID NO: 1284 - RNA, 25 nt, Artificial Sequence
  Description of Artificial Sequence Synthetic oligonucleotide
aagcuugcauuauugauuuaucauc

SEQ ID NO: 1285 - RNA, 25 nt, Artificial Sequence
  Description of Artificial Sequence Synthetic oligonucleotide
uaagcuugcauuauugauuuaucau

SEQ ID NO: 1286 - RNA, 25 nt, Artificial Sequence
  Description of Artificial Sequence Synthetic oligonucleotide
guaagcuugcauuauugauuuauca

SEQ ID NO: 1287 - RNA, 25 nt, Artificial Sequence
  Description of Artificial Sequence Synthetic oligonucleotide
aguaagcuugcauuauugauuuauc

SEQ ID NO: 1288 - RNA, 25 nt, Artificial Sequence
  Description of Artificial Sequence Synthetic oligonucleotide
uaguaagcuugcauuauugauuuau

SEQ ID NO: 1289 - RNA, 25 nt, Artificial Sequence
  Description of Artificial Sequence Synthetic oligonucleotide
auaguaagcuugcauuauugauuua

SEQ ID NO: 1290 - RNA, 25 nt, Artificial Sequence
  Description of Artificial Sequence Synthetic oligonucleotide
gauaguaagcuugcauuauugauuu

SEQ ID NO: 1291 - RNA, 25 nt, Artificial Sequence
  Description of Artificial Sequence Synthetic oligonucleotide
ugauaguaagcuugcauuauugauu

SEQ ID NO: 1292 - RNA, 25 nt, Artificial Sequence
  Description of Artificial Sequence Synthetic oligonucleotide
augauaguaagcuugcauuauugau

SEQ ID NO: 1293 - RNA, 25 nt, Artificial Sequence
  Description of Artificial Sequence Synthetic oligonucleotide
aaugauaguaagcuugcauuauuga

SEQ ID NO: 1294 - RNA, 25 nt, Artificial Sequence
  Description of Artificial Sequence Synthetic oligonucleotide
aaaugauaguaagcuugcauuauug

SEQ ID NO: 1295 - RNA, 25 nt, Artificial Sequence
  Description of Artificial Sequence Synthetic oligonucleotide
uaaaugauaguaagcuugcauuauu

SEQ ID NO: 1296 - RNA, 25 nt, Artificial Sequence
  Description of Artificial Sequence Synthetic oligonucleotide
auaaaugauaguaagcuugcauuau

SEQ ID NO: 1297 - RNA, 25 nt, Artificial Sequence
  Description of Artificial Sequence Synthetic oligonucleotide
cauaaaugauaguaagcuugcauua

SEQ ID NO: 1298 - RNA, 25 nt, Artificial Sequence
  Description of Artificial Sequence Synthetic oligonucleotide
ucauaaaugauaguaagcuugcauu

SEQ ID NO: 1299 - RNA, 25 nt, Artificial Sequence
  Description of Artificial Sequence Synthetic oligonucleotide
uucauaaaugauaguaagcuugcau

SEQ ID NO: 1300 - RNA, 25 nt, Artificial Sequence
  Description of Artificial Sequence Synthetic oligonucleotide
auucauaaaugauaguaagcuugca

SEQ ID NO: 1301 - RNA, 25 nt, Artificial Sequence
  Description of Artificial Sequence Synthetic oligonucleotide
uauucauaaaugauaguaagcuugc

SEQ ID NO: 1302 - RNA, 25 nt, Artificial Sequence
  Description of Artificial Sequence Synthetic oligonucleotide
cuauucauaaaugauaguaagcuug

SEQ ID NO: 1303 - RNA, 25 nt, Artificial Sequence
  Description of Artificial Sequence Synthetic oligonucleotide
gcuauucauaaaugauaguaagcuu

SEQ ID NO: 1304 - RNA, 25 nt, Artificial Sequence
  Description of Artificial Sequence Synthetic oligonucleotide
ugcuauucauaaaugauaguaagcu

SEQ ID NO: 1305 - RNA, 25 nt, Artificial Sequence
  Description of Artificial Sequence Synthetic oligonucleotide
uugcuauucauaaaugauaguaagc

SEQ ID NO: 1306 - RNA, 25 nt, Artificial Sequence
  Description of Artificial Sequence Synthetic oligonucleotide
auugcuauucauaaaugauaguaag

SEQ ID NO: 1307 - RNA, 25 nt, Artificial Sequence
  Description of Artificial Sequence Synthetic oligonucleotide
uauugcuauucauaaaugauaguaa

SEQ ID NO: 1308 - RNA, 25 nt, Artificial Sequence
  Description of Artificial Sequence Synthetic oligonucleotide
guauugcuauucauaaaugauagua

SEQ ID NO: 1309 - RNA, 25 nt, Artificial Sequence
  Description of Artificial Sequence Synthetic oligonucleotide
aguauugcuauucauaaaugauagu

SEQ ID NO: 1310 - RNA, 25 nt, Artificial Sequence
  Description of Artificial Sequence Synthetic oligonucleotide
caguauugcuauucauaaaugauag

SEQ ID NO: 1311 - RNA, 25 nt, Artificial Sequence
  Description of Artificial Sequence Synthetic oligonucleotide
ucaguauugcuauucauaaaugaua

SEQ ID NO: 1312 - RNA, 25 nt, Artificial Sequence
  Description of Artificial Sequence Synthetic oligonucleotide
uucaguauugcuauucauaaaugau

SEQ ID NO: 1313 - RNA, 25 nt, Artificial Sequence
  Description of Artificial Sequence Synthetic oligonucleotide
cuucaguauugcuauucauaaauga

SEQ ID NO: 1314 - RNA, 25 nt, Artificial Sequence
  Description of Artificial Sequence Synthetic oligonucleotide
ucuucaguauugcuauucauaaaug

SEQ ID NO: 1315 - RNA, 25 nt, Artificial Sequence
  Description of Artificial Sequence Synthetic oligonucleotide
uucuucaguauugcuauucauaaau

SEQ ID NO: 1316 - RNA, 25 nt, Artificial Sequence
  Description of Artificial Sequence Synthetic oligonucleotide
uuucuucaguauugcuauucauaaa

SEQ ID NO: 1317 - RNA, 25 nt, Artificial Sequence
  Description of Artificial Sequence Synthetic oligonucleotide
auuucuucaguauugcuauucauaa

SEQ ID NO: 1318 - RNA, 25 nt, Artificial Sequence
  Description of Artificial Sequence Synthetic oligonucleotide
aauuucuucaguauugcuauucaua

SEQ ID NO: 1319 - RNA, 25 nt, Artificial Sequence
  Description of Artificial Sequence Synthetic oligonucleotide
uaauuucuucaguauugcuauucau

SEQ ID NO: 1320 - RNA, 25 nt, Artificial Sequence
  Description of Artificial Sequence Synthetic oligonucleotide
uuaauuucuucaguauugcuauuca

SEQ ID NO: 1321 - RNA, 25 nt, Artificial Sequence
  Description of Artificial Sequence Synthetic oligonucleotide
uuuaauuucuucaguauugcuauuc

SEQ ID NO: 1322 - RNA, 25 nt, Artificial Sequence
  Description of Artificial Sequence Synthetic oligonucleotide
uuuuaauuucuucaguauugcuauu

SEQ ID NO: 1323 - RNA, 25 nt, Artificial Sequence
  Description of Artificial Sequence Synthetic oligonucleotide
guuuuaauuucuucaguauugcuau

SEQ ID NO: 1324 - RNA, 25 nt, Artificial Sequence
  Description of Artificial Sequence Synthetic oligonucleotide
uguuuuaauuucuucaguauugcua

SEQ ID NO: 1325 - RNA, 25 nt, Artificial Sequence
  Description of Artificial Sequence Synthetic oligonucleotide
uuguuuuaauuucuucaguauugcu

SEQ ID NO: 1326 - RNA, 25 nt, Artificial Sequence
  Description of Artificial Sequence Synthetic oligonucleotide
uuuguuuuaauuucuucaguauugc

SEQ ID NO: 1327 - RNA, 25 nt, Artificial Sequence
  Description of Artificial Sequence Synthetic oligonucleotide
uuuuguuuuaauuucuucaguauug

SEQ ID NO: 1328 - RNA, 25 nt, Artificial Sequence
  Description of Artificial Sequence Synthetic oligonucleotide
cuuuuguuuuaauuucuucaguauu

SEQ ID NO: 1329 - RNA, 25 nt, Artificial Sequence
  Description of Artificial Sequence Synthetic oligonucleotide
ucuuuuguuuuaauuucuucaguau

SEQ ID NO: 1330 - RNA, 25 nt, Artificial Sequence
  Description of Artificial Sequence Synthetic oligonucleotide
aucuuuuguuuuaauuucuucagua

SEQ ID NO: 1331 - RNA, 25 nt, Artificial Sequence
  Description of Artificial Sequence Synthetic oligonucleotide
aaucuuuuguuuuaauuucuucagu

SEQ ID NO: 1332 - RNA, 25 nt, Artificial Sequence
  Description of Artificial Sequence Synthetic oligonucleotide
caaucuuuuguuuuaauuucuucag

SEQ ID NO: 1333 - RNA, 25 nt, Artificial Sequence
  Description of Artificial Sequence Synthetic oligonucleotide
gcaaucuuuuguuuuaauuucuuca

SEQ ID NO: 1334 - RNA, 25 nt, Artificial Sequence
  Description of Artificial Sequence Synthetic oligonucleotide
agcaaucuuuuguuuuaauuucuuc

SEQ ID NO: 1335 - RNA, 25 nt, Artificial Sequence
  Description of Artificial Sequence Synthetic oligonucleotide
cagcaaucuuuuguuuuaauuucuu

SEQ ID NO: 1336 - RNA, 25 nt, Artificial Sequence
  Description of Artificial Sequence Synthetic oligonucleotide
acagcaaucuuuuguuuuaauuucu

SEQ ID NO: 1337 - RNA, 25 nt, Artificial Sequence
  Description of Artificial Sequence Synthetic oligonucleotide
gacagcaaucuuuuguuuuaauuuc

SEQ ID NO: 1338 - RNA, 25 nt, Artificial Sequence
  Description of Artificial Sequence Synthetic oligonucleotide
agacagcaaucuuuuguuuuaauuu

SEQ ID NO: 1339 - RNA, 25 nt, Artificial Sequence
  Description of Artificial Sequence Synthetic oligonucleotide
gagacagcaaucuuuuguuuuaauu

SEQ ID NO: 1340 - RNA, 25 nt, Artificial Sequence
  Description of Artificial Sequence Synthetic oligonucleotide
ugagacagcaaucuuuuguuuuaau

SEQ ID NO: 1341 - RNA, 25 nt, Artificial Sequence
  Description of Artificial Sequence Synthetic oligonucleotide
uugagacagcaaucuuuuguuuuaa

SEQ ID NO: 1342 - RNA, 25 nt, Artificial Sequence
  Description of Artificial Sequence Synthetic oligonucleotide
auugagacagcaaucuuuuguuuua

SEQ ID NO: 1343 - RNA, 25 nt, Artificial Sequence
  Description of Artificial Sequence Synthetic oligonucleotide
uauugagacagcaaucuuuuguuuu

SEQ ID NO: 1344 - RNA, 25 nt, Artificial Sequence
  Description of Artificial Sequence Synthetic oligonucleotide
auauugagacagcaaucuuuuguuu

SEQ ID NO: 1345 - RNA, 25 nt, Artificial Sequence
  Description of Artificial Sequence Synthetic oligonucleotide
uauauugagacagcaaucuuuuguu

SEQ ID NO: 1346 - RNA, 25 nt, Artificial Sequence
  Description of Artificial Sequence Synthetic oligonucleotide
auauauugagacagcaaucuuuugu

SEQ ID NO: 1347 - RNA, 25 nt, Artificial Sequence
  Description of Artificial Sequence Synthetic oligonucleotide
gauauauugagacagcaaucuuuug

SEQ ID NO: 1348 - RNA, 25 nt, Artificial Sequence
  Description of Artificial Sequence Synthetic oligonucleotide
agauauauugagacagcaaucuuuu

SEQ ID NO: 1349 - RNA, 25 nt, Artificial Sequence
  Description of Artificial Sequence Synthetic oligonucleotide
aagauauauugagacagcaaucuuu

SEQ ID NO: 1350 - RNA, 25 nt, Artificial Sequence
  Description of Artificial Sequence Synthetic oligonucleotide
uaagauauauugagacagcaaucuu

SEQ ID NO: 1351 - RNA, 25 nt, Artificial Sequence
  Description of Artificial Sequence Synthetic oligonucleotide
auaagauauauugagacagcaaucu

SEQ ID NO: 1352 - RNA, 25 nt, Artificial Sequence
  Description of Artificial Sequence Synthetic oligonucleotide
uauaagauauauugagacagcaauc

SEQ ID NO: 1353 - RNA, 25 nt, Artificial Sequence
  Description of Artificial Sequence Synthetic oligonucleotide
auauaagauauauugagacagcaau

SEQ ID NO: 1354 - RNA, 25 nt, Artificial Sequence
  Description of Artificial Sequence Synthetic oligonucleotide
aauauaagauauauugagacagcaa

SEQ ID NO: 1355 - RNA, 25 nt, Artificial Sequence
  Description of Artificial Sequence Synthetic oligonucleotide
aaauauaagauauauugagacagca

SEQ ID NO: 1356 - RNA, 25 nt, Artificial Sequence
  Description of Artificial Sequence Synthetic oligonucleotide
uaaauauaagauauauugagacagc

SEQ ID NO: 1357 - RNA, 25 nt, Artificial Sequence
  Description of Artificial Sequence Synthetic oligonucleotide
auaaauauaagauauauugagacag

SEQ ID NO: 1358 - RNA, 25 nt, Artificial Sequence
  Description of Artificial Sequence Synthetic oligonucleotide
aauaaauauaagauauauugagaca

SEQ ID NO: 1359 - RNA, 25 nt, Artificial Sequence
  Description of Artificial Sequence Synthetic oligonucleotide
uaauaaauauaagauauauugagac

SEQ ID NO: 1360 - RNA, 25 nt, Artificial Sequence
  Description of Artificial Sequence Synthetic oligonucleotide
auaauaaauauaagauauauugaga

SEQ ID NO: 1361 - RNA, 25 nt, Artificial Sequence
  Description of Artificial Sequence Synthetic oligonucleotide
aauaauaaauauaagauauauugag

SEQ ID NO: 1362 - RNA, 25 nt, Artificial Sequence
  Description of Artificial Sequence Synthetic oligonucleotide
aaauaauaaauauaagauauauuga

SEQ ID NO: 1363 - RNA, 25 nt, Artificial Sequence
  Description of Artificial Sequence Synthetic oligonucleotide
uaaauaauaaauauaagauauauug

SEQ ID NO: 1364 - RNA, 25 nt, Artificial Sequence
  Description of Artificial Sequence Synthetic oligonucleotide
guaaauaauaaauauaagauauauu

SEQ ID NO: 1365 - RNA, 25 nt, Artificial Sequence
  Description of Artificial Sequence Synthetic oligonucleotide
gguaaauaauaaauauaagauauau

SEQ ID NO: 1366 - RNA, 25 nt, Artificial Sequence
  Description of Artificial Sequence Synthetic oligonucleotide
ugguaaauaauaaauauaagauaua

SEQ ID NO: 1367 - RNA, 25 nt, Artificial Sequence
  Description of Artificial Sequence Synthetic oligonucleotide
uugguaaauaauaaauauaagauau

SEQ ID NO: 1368 - RNA, 25 nt, Artificial Sequence
  Description of Artificial Sequence Synthetic oligonucleotide
uuugguaaauaauaaauauaagaua

SEQ ID NO: 1369 - RNA, 25 nt, Artificial Sequence
  Description of Artificial Sequence Synthetic oligonucleotide
auuugguaaauaauaaauauaagau

SEQ ID NO: 1370 - RNA, 25 nt, Artificial Sequence
  Description of Artificial Sequence Synthetic oligonucleotide
aauuugguaaauaauaaauauaaga

SEQ ID NO: 1371 - RNA, 25 nt, Artificial Sequence
  Description of Artificial Sequence Synthetic oligonucleotide
uaauuugguaaauaauaaauauaag

SEQ ID NO: 1372 - RNA, 25 nt, Artificial Sequence
  Description of Artificial Sequence Synthetic oligonucleotide
auaauuugguaaauaauaaauauaa

SEQ ID NO: 1373 - RNA, 25 nt, Artificial Sequence
  Description of Artificial Sequence Synthetic oligonucleotide
aauaauuugguaaauaauaaauaua

SEQ ID NO: 1374 - RNA, 25 nt, Artificial Sequence
  Description of Artificial Sequence Synthetic oligonucleotide
gaauaauuugguaaauaauaaauau

SEQ ID NO: 1375 - RNA, 25 nt, Artificial Sequence
  Description of Artificial Sequence Synthetic oligonucleotide
agaauaauuugguaaauaauaaaua

SEQ ID NO: 1376 - RNA, 25 nt, Artificial Sequence
  Description of Artificial Sequence Synthetic oligonucleotide
uagaauaauuugguaaauaauaaau

SEQ ID NO: 1377 - RNA, 25 nt, Artificial Sequence
  Description of Artificial Sequence Synthetic oligonucleotide
uuagaauaauuugguaaauaauaaa

SEQ ID NO: 1378 - RNA, 25 nt, Artificial Sequence
  Description of Artificial Sequence Synthetic oligonucleotide
cuuagaauaauuugguaaauaauaa

SEQ ID NO: 1379 - RNA, 25 nt, Artificial Sequence
  Description of Artificial Sequence Synthetic oligonucleotide
ucuuagaauaauuugguaaauaaua

SEQ ID NO: 1380 - RNA, 25 nt, Artificial Sequence
  Description of Artificial Sequence Synthetic oligonucleotide
cucuuagaauaauuugguaaauaau

SEQ ID NO: 1381 - RNA, 25 nt, Artificial Sequence
  Description of Artificial Sequence Synthetic oligonucleotide
acucuuagaauaauuugguaaauaa

SEQ ID NO: 1382 - RNA, 25 nt, Artificial Sequence
  Description of Artificial Sequence Synthetic oligonucleotide
uacucuuagaauaauuugguaaaua

SEQ ID NO: 1383 - RNA, 25 nt, Artificial Sequence
  Description of Artificial Sequence Synthetic oligonucleotide
auacucuuagaauaauuugguaaau

SEQ ID NO: 1384 - RNA, 25 nt, Artificial Sequence
  Description of Artificial Sequence Synthetic oligonucleotide
aauacucuuagaauaauuugguaaa

SEQ ID NO: 1385 - RNA, 25 nt, Artificial Sequence
  Description of Artificial Sequence Synthetic oligonucleotide
aaauacucuuagaauaauuugguaa

SEQ ID NO: 1386 - RNA, 25 nt, Artificial Sequence
  Description of Artificial Sequence Synthetic oligonucleotide
gaaauacucuuagaauaauuuggua

SEQ ID NO: 1387 - RNA, 25 nt, Artificial Sequence
  Description of Artificial Sequence Synthetic oligonucleotide
agaaauacucuuagaauaauuuggu

SEQ ID NO: 1388 - RNA, 25 nt, Artificial Sequence
  Description of Artificial Sequence Synthetic oligonucleotide
aagaaauacucuuagaauaauuugg

SEQ ID NO: 1389 - RNA, 25 nt, Artificial Sequence
  Description of Artificial Sequence Synthetic oligonucleotide
gaagaaauacucuuagaauaauuug

SEQ ID NO: 1390 - RNA, 25 nt, Artificial Sequence
  Description of Artificial Sequence Synthetic oligonucleotide
ggaagaaauacucuuagaauaauuu

SEQ ID NO: 1391 - DNA, 55361 bp, Homo sapiens
agctcctaggaagcttcagggcttaaagctccactctacttggactgtactatcaggcccccaaaatggggggagccgacagggaaggactgatttccatttcaaactgcattctggtactttgtactccagcaccattggccgatcaatatttaatgcttggagattctgactctgcgggagtcatgtcaggggaccttgggagccaatctgcttgagcttctgagtgataattattcatgggctcctgcctcttgctctttctctagcacggtcccactctgcagactcagtgccttattcagtcttctctctcgctctctccgctgctgtagccggaccctttgccttcgccactgctcagcgtctgcacatccctacaatggctaaaacagcaatggtaaggcactgcgcctcgttctccgtcggctctacctggagcccacctctcacctcctctcttgagctctagaagcattcagagatattttataaagaaaaagatgttaatggtaacacaggaccaggaaggacagggcagttctgggggaggtgggagggcagagaagaggtctatggaaatctaaagcgaagaatttcttttaaaaggtagaagcgggtaagttgccctcctatgggtagagaatttattctgtttccatatttaaaattaggactcaatcgtgaggggaggaagctaccttaactgtttgccttaaatgggcttaagggacattttggaaagtgctttataacgaccttttttttttttatttcttctctagtttaagaagaaaataggaaaggggtaaagggaaggtgggagaaaggaaaaagaaaattgcaaagtcaaagcggtcccatcccgctgtttgaaagatgggtggagacggggggaggggatggagagaactgggcacattttacggtattgtctcgtcgaagaaaccgctagtcctggggtgcggtgcagggaggtaagacggcgggggacagggtgggggtaggacctccgctcctttgttttagggcaagggaggggaaggagagaggaagtcgcggagggcgtggagggcgcgggtgggcagctgcaggggcggggaagcgcgcggcagggaggggtggagggacagcggcttcgaaggcgctggggtggggtttctttgtgtgcggaccagcggtcccggggggaggcacctgcagcgctgggcgcacaatgcggacagccccacccagtgcggaaccgcgcagccccgcccccccgcccggtgctgcatcttcattcgaaagggggtcgggtggggagcgcagcgtgacacccaggagcccaaccctgcggggacagcggcgccacgccccgcgctccccgctcccgactccccgccgcggcttccaagagagacctgaccactgaccccgccctccccacgctggcctcattgttctgcttttaagagagatgggaaaagtgggttaacatttttcttttcggaagcaaattacatagagtgtttagacatagacacagataaagggttctttgaagacctttgatcgtttgcgggaaaagcttctagaacctagacatgtgtatgtataataatagagatgacatgaaatcgtatataaagcaaaagaggtcaaagtcttaagttaagccacgcgaaatttccgttttgtgggtcagacagtgccaaatatcggcaatttcataagctcagagagacaagacagtggagacacaggatgaccggaaaagattctggattcagggccttcatccgcaattggtcttgtgccttgagtgcccacggttctggcgctcagtggccccggggtgaaaaggcagggtggggcctggggtcctgtggcagctggaagcacgtgtcccccgggacttggttgcaggatgcggagacagggaaagctgccgaaaggactccatctgcgcggctccgccctgccctaccctccccgcggagccggggagacctcaggctccgagactggcggggaagaggaatatgggaggggcagttgagctgtatgcagtcctggaacctcttttttcagccccgcagtccacaacggcccgagcaccccttgatgtgcgcagacccccggcgtggctctcagccccagcaccgagcccctcccagccaagcgggtggctctgcagaaaagctggctcgagccccgcccggccacacaaaggcgcggccccacccagcccgggcgcgagaccgcagaggtgacccccttcccagggattcagggagggctgtctcttctcgcccacccacggtccgcggagctcggggctttttttcccccagcccaagccccccgcccaccctctgttctctatgattttccagaatggagaccccgcgaggggcttctctaagggagaccctcgctcctccagcggggcgcggctcggccccacccctcccagctgaggcccagagccgcctaccgctggccgggtgggggcgcacgtggcgactgggtgtgtggagcgcagccagccctgcagagccccgcgccgcgccctgcgctcccctccccggagttgggcgctcgcccccgcggtgcagccggggagaccggtttctgcgcagtgtcctgagctacccccgctttccacaattcgcagttcactcgcacgtccagaaaggttctgagaatgggtggtgggggcgatctcgcctcgctttctgcacccctcagaaaggtttccgctgcaggctagtggctgcaaactcatcgtcatcatcagtattattatcatttcaaatcgttgttattatttaatgattcagtagccttgtttgttctcatttgttcaaaagggacgtggattgctcttggttaaggattaacccttgttgcgttcgctttgcttcctcctaattgccctcatccctttcccccacaaaaaggtaaatttgtctccagttgttcattttaagttataaagcaaatatatttttgcttcctgccaggattatgtatgttcatgtggctaagatacatgtgcaagtgcttgctaagagcagggtttgtgtgccaacgattgctggaaaattctctgcaaagaattgtttgtggctgcaatgggtgagaatacacatatataattgagatgatcttcaacataaggttatatctataaatatataaatatagtttatgcacaaaattttaagttttttcccctgaaactgttcttccaactgctgattcttgatacagcctcaatcctacacagatacatggatcgtgaaatggtagccgccatccaaataaaaatcccaccccaaatatgacaaacgcaagcatcctttctggccataatttaactgcatttgcaaatcatgaaaaaaacactacttctgcagtattaaaataatagattttgaaattaattccaatttcaaagataattaattatcagggcgagtgcttttttcctgattcattaaacaattatgtattcagcatgattgtaagaggtgcatataatattccccattatcttttctaatgaagtgggcaccttctgaatggatatataagtaactagaaatgaaaagctgaggatttggtcagaatttcaggataaaactgaaagaaatggcagtagtttatcaattaatctcatgtatttagtttataccaggtgagtaagctgagcctgcaataaacactctctgtcccagtgtaacacgtcgcaggtagctagaatgataggataaattaatagaccttgtggtgtttgtctatgcacgttaaaattctctgagagaaagtatattttaaaatgataattaagattggacatttgtgctattaaaatctacaactttagtcaaaattcacaatggtttttttttacaataatgtgacttacagatttgtagtaaattattctattctaaaagagaaatgagtgtttttattgttacagctattacctcattaatatttttagcaaacttttatttgttgcattgaaagcagttttaattactttgggtttttatttttcaaattactaatggatagatggtggaataagcatttaatcatttggcacaatatgacttccatcaaatagctcattctcagtgattaaaaaatgctacaagaggctacaatttactcagattcaggaaatgtcctttcagagtgccataaggctgattcatataataaaatagttttcttccctataatttaagatcaaatagttacttagttctgtgaatacctagcagtagctatcaaacagaattttaaagttaaatctgtacaactaacaatgaagtggaggatgaatcgatacatattgaatggaagactttgtcattgataaattcaggccatctttaggaaaattccggatttatcaatcaccattattttttacttcaactgagtgtgactgatcacatgctcaggctaccttggtagctcattgctcacaggaggctgaaaaaagctggcctccgagcaggaggaagctcagagcacaaacctaggcctgggcgtggccactgggagctgctgatagcgaaccccagctcacaccagtttcttttttggtcgtgggaagaaaaacacatattatcctgttgtcacaagatctgtgaccttatatgaaaaaatgctagaattttttcattaaaaaagaaaatactgaactagccagtgacccagatgttttcagaacctagactggttctgtccattggaaaacctcggtgtctgcattaacttttcaccacactagagggcaatcatgttctctaaaaaagcagatgattgatgtaaacctagttccaaatattaactgtttaataaaatcttttcttttaccaggaacattcaagtgtttattcaataagctgatgccatgctttaccctagtggatgaacagagcttgtacaattttcaaggagacaggatgaaatgagtggtcataatctgaaagtagatacacgccctggttaattattccctgatggttttacttctcagttttattacattgttattataataccatttatgttacttctgagattttgtagtggataaatagtagaaaaatgtcagtagtaatagcaaagttatttagcagccgaatattttaatgcttaaaaataaaggaataaattaaagaaaatcattgtttacttcttcatcgattgaaatgtgccccctgttcagagcacatctgaatatcagagtctccacctgcagagaacatgcagcttagcgagtaaaacaggcaggtatgtgatactgaggaggtgtaccaaaaactgactgctgttatttttcccatcttctaagtctgtctttcttttccatttaaagatacctttttaaatctaatccaatgtgatttcaatctagttttatcagatttcaacaattattgagcatctccttgtagtggttttctgtttattagaaaatcgatgttaattttaacgaagtaagaagaaatatataagtataaactaattttgggtatcatcaaaagtggattttttaaatatgcattgatagaattattttttgattacattttatgtaattctaatccagctataaaatatttaatagtgtcatattactgtgttcctcaaactttgatgtgcatatgaattacctttgattttcattaaaatgcaaattctgattcaatacatctggcttgaggcagacattctgtcttccgaacaagctcccagatgatgctgattctgaccactaaacacatcagttttagggatattaacttgtaatatacaggtatccctcctggtaagctctggtattatgtcttaacatttttaaatctatggtaatctttacaaaatattttacttccgaactcatatacctggggattttattactctgggaattatgtgttctgccccatcactctctcttaattggatttttaaaattatattcatattgcaggactcggcagaagaccttcgagagaaaggtagaaaataagaatttggctctctgtgtgagcatgtgtgcgtgtgtgcgagagagagagacagacagcctgcctaagaagaaatgaatgtgaatgcggcttgtggcacagttgacaaggatgataaatcaataatgcaagcttactatcatttatgaatagcaatactgaagaaattaaaacaaaagattgctgtctcaatatatcttatatttattatttaccaaattattctaagagtatttcttcctgaataccatgtgagaaaattcttaagaatttattgagtatgactgtatatttgaaaagagtgttttcttctgcttatctaagccaataaaggatcttcattattcaattctaactttctaaggaagtcaacctacagatcagaaagaggatcttcaaggaatagcatcaaagacatagtcaggtctcccatgcagtgactggctgaccatgcagccattaccacctttctggaaatattatgctgcaaaaatgatacaatacacgaaatatctcaaattaaaaaatataacatttcccaaatagggcactaaaaacatgatcccaaataaaactagcttcagggtttgcagaatatactgttactcaacacaaagttggactaagtctcaaagttagccattcagttgttgttaacagttcatttcagggtctctcagaagctgggaaactttccatttttgcaatttcttgtacattgaaggaaaggaagacacacttaagacagcattacaaaagtaattcatgttttaaatgtttaattctggcagtcgggcagggctctctgtataacctcatttggagatgacaaaaatctaaacttgagggcctcgagccaataagtcttcctatttctttactcaaacattttcccgcaatggtgctttctttcaactgtttttctggtgtattcataaattccagattctctatgggaagtaacttttattgattgatttaacccttgtatagcacatataacatgcaaggcattgttctaagaactttccacatattaactgtgttaatcacttaataatcctaagtaggttctattacagatatggaaactgaggcacagaaagttgaagtatcttactcaaggtcacacagttagtcagatccagaatttgggcccaggccatctggcttcggaatccatctttcaccgattgctgctagtctcatatctgttccatgttagaggtgagctcccattgcagaggtcacacctgtgatatcaccattttatttaaacagaccagagatggtcttctcctttctgatcacagactcaccttgaagagaaaatacttccaaattgatgcctagttttaatagcttacctggggcttattcaaataattgccatgatttaggctttgggagaaagagagctatgaggccgtgtgggttgtaacgtatgagacacatggcgttctgcaggctcagcacagcatcgatttctggtgggaacacactctgatgaccagttccagaaataacattgacttaatctcctcagtcccatcatggttagcacatttcaaaatgcctccttaactacttccataggccagagatatttagttttaacattttgttgaataaaataaatttacacattcacatttaatataactattagatgttatttcaagattctcttcatattaccatcaaagcaggcaggcaggcaggagagaactgtaggaaggttttgaatcccttgtgaaacatttttaattatcttttaataaaggaatcaggccctgtcatttgtcaaggagacatttgcagtagtaaagcttgtgtttataatatccatttttattagtcatgattaaagataacatttgtgtacatttgttctcacaaaacacttttatatgagtgtaaaggttaattaatgcatttcagccatcattttgctggtcatgtggaaatatagcttctttaggaattgtacttagagtaggagccacatattatactataaaaccataacaaaaatattttaagtttgttctcacttgttgttgacctccagagtaaaatatttaatactctggaaagttatgggtttcaaaatttattttatggcaagaaatagataattacagttctcatagagcacatttaaaataatttatttttatagggcaaaaatattgcctaggactgaatgatttttttttttttacaaagattgtaaagcaacgcctgcaagagtgcccatttagcagttattcttctggaataattgtattttggatgttggagttcgcacattaaccattagtacaagtacccaatataacaatagatcatcaggataataaatctgtccatcttttagttgtatgtctttatatcaggataaagagaattgagtgaaatttatctaaacctagtcccacaaatacttttacaagagagcatgttaaagtgtaaattaaatttttattagcattctactctgtctttggaagttttttttccttatgaaatgcagccataaagtttaacttccattaacaaagctgctcacagtaaacctattataataatagtttcccagtttgggcttcctagtgaggagcaacctaactcacacgaaacaaccccaacttataatatattgactgttacaaaactgagaccagaaaatcccatcaagatggtactgttatcatttccagactctcgggaagaacattaatcatctcaggcacttttaggatagacttattgcagcctccctgggaactctgcttcagaacataattatttttattaatgcagagttactttttatttccaacaaaaatatctattgttattatttaagtcttacagctttatctgagaaattccaattagcacccttctcataataaatattcaaacacatgaaaaattaccaaagttgttctagtcttttaatgacatattacatgatcctgcactcttgtcactttaaaaattatctttttattatatttctgatgatttttttcttatatagttttttaaaaggagcaggcaagcatagaagactaaaaaatgttcaaaagaaaaattaaatcgcatgatctatctatatgggaccttgtcatttttagaaaacattcacctgcttcatccttttgaatcttcatataatccctctgagatgggcatactatacaagttgtcttatttaaagattggtaaatttaagctcaaataatttattcagtggcaagcctcagaggcagactcggaacacaggtctaatatatattatatatatattataacatataatatatatattacatataataaagttgtgtatattatttacctatcaaaatatttatatgtaatatataaatatgttatatatcatgtatgtgcctatttcatacatatatacacattcatgcaaaataaggtttagcactccctccactgtcctgtaataaaacatgcacagtgagaatagtcatacacgaggcatatttgtcttcagtttaaagtcattgatagtcagtgtcactaactaaagtaaaatagattggagcaccaactttgttctgaagcctgtgccaggtattatgagaacaaaaataaaaatgttcctcacccttggtggatttagtcttttgcagaaaaaaagatcctgtacatgtcagaaagttcaatagtaataatggtaatttataactataaatggaagtcaccatctcacaatttcaccatcttaacaattttgttaaactgccctacaatattacaagatagtacataatgatacactagtaacatcaactaggaagtaccaagatccaccaaaaggctgaaaaatttaaatatttaatgagtccatcaaccaatctggccagagaattctttaattaaaatgcttcccaaattttactgagaatcagcagcgtttgaggagctagcctccacccccagaggttctcactctattaggtctgaagcaggtcccatggatttgcatttctaacaagctcccaggtggtgctgatgaggctgattcagaaccacacttggagtagacctaaaacagcagtgacctgtagggtccccaagcagcaggccaggacagcatgtgagttacgtcctctgtggagctctgcaacaaggcgtcaagaggtcagagtctaagtccccatcagctctgcccttctccaccagtgctgctggtgctgcatggaaggaagagcccagaagggattctgagtttcagtctttactcttgctgacgcaccttggtcaggtcaattttcctgtttgttcctctaattcagcatctgtaaaatagccatgtgaactgccttgtccatatcagagggtctttttcagactcaaggaaaaaaacgtgaaagtgattagtgtctgtcaagtagtatataaatgcaagaagttgagtttttaaattgtcattagatataaatacccatgtgcatgcatttagaatgagtaaagagggaacaaggagcgcaatcaaaaactgcgtcatttgctttttgaaaaatactttctatgtaatgaaaagtgaaataaaatgttaattgagtccctctgacaacagcatcagacgttttgcagttcttgtgattagaacccacctggccagcccttcttcctcctaaagaagagccttcttcttcttaaatgaaggttggctcagaagaagcaattaactcattcaacgttttgttacagtcaatccacatccaacttttccccaactcaatctgctttaagggaaggatggtaagtggtggcccaagatggcaaccatcaagcttagagaatctctagaagcaggggtgtccccagcaagtagacactgaaaatatgagagggctgataagccagagataaaactcagtacttactttgcttctagtccatgtctacccctttcttggcaccaccttgacactaccctctgagtccaccttcctgagatggtacaaactctgcttagacaaagcagcccatgtccaaaggtgttagggctcagtttaaagctgccttcaaaagttaaaacagaagtgtaaagttctgtgcaattaaaaataatcagcttgtcttggaactcaaacgaatgtaaaatcctatgaaaattaaaaagcagtaccacaagttaccccaaaagtccttaggtcagtaactgttcctgttacaggtaagagagagcatggattagaggtgggcgtgggtatccagtggacatggttttgaaccatgctccactactactcactatctgagaattcttaaatttattaatcatttctatattataattttctcagttatgaaatgggaaaacaatacctaaatcacatggttgttaagtaagcaattgattgttaagcatttggtcatcaaaaatattaatccccttccctgattccctagataaatgatgaaaatactaaataaaaataataaaaatttaaagtgaacatctcaattcttatactttgttaatttctacatgtattacaaatctactagaaattacttggaattgaggaaatgattactgcttaataattctttgtggtagagggagagttggtatcatatttatgagacagcagccaatatagtatatctcaaaggaaaaaatccattctacataatgccagaatttaatagttaagcattttatctaggtcacagcacaataagcaagatggataattaaaataaaagtatatttctcttgcatatatttctcatttcatgtttccctatcatattttatatcttaccttacttcaaatacatatataccttcaataaaactgagccttcttgcttacccaggaagtttcatcattcagtagaaataaaagatgactttagaaatattaaaatacaaaaatctacactgaggtcttttgaatgcaggaaaaagaattatatcacacacacacgtacacgcacgcatgcatacacacacacagaacctctcgttctttcttaacatcttatcaatccatcagtttcactcccactccgtatcacctgactgtgcacaatatctcattgccacctcccagtcttctccctgcctggcaccctcctgctctcctgcttccactttaaacacccttccttcagctaggtcttttctttcagggatcctcccgttgctttcttatctggatcaatttagccttcctcttctccacccattagtggataagcacgacaaagacactagagtcaaataatacaaacagaatataccttagatgagtatggtgatgaaaaggatatggatacttagagtttagcactattctctcagccactcaggaaagcaacgcctttacaatcaatagtgtttcaggtaccaatcaataatctgttattgctatttttaaaatctataaggtatcagtaaaatgtaattactagagcaacaaagatatcttgtgaaatcaaattagtattcatccagcaactgagtacaaaggtttaagggaggataactaccaataccaaaacattttaagcattttgttttgcctcctaaatatcaaatcatgtaaatgtgtggtacataaattaggaattatatttatgacatagctgcagacatattaagagaaatatgtgcttatatttacaagtatagtacagttctttttcatattagatactgttgatgataatctgcatataaaaatgctcaatattttttcacatttataagccataaaatacagctaataaaatgtgtttctactttctcataaacatggaatagtgacaaacaaggagctttatatgaaagcaccattacaatttaaactctcacaaggtcataatatattgcactaagcaggagagttcagcttatttaaaaaaaaaaataaactctaatgaggttctggaatgcagagccaaagcataaagatggaaataaaagaattgcatgtcttctgaactgacttggttgatgatttttttaaaaaaggttttgtgtcttctgacttggttgatgattttttaaaaaaacgttttgtggtagaacaaataaggtaaatgaaattcagtatttaggatgaaaagtttttctaatttcaggaacaacattgaagaaatattgaactaagcagctttgaaagaatcagattccatttgttgaaatttttctgagaatgaatttttttaagacagtgtacacagttgcagtgtgtattggttatggattgtggcaagctatattacaacttacccaagaaataaggaggctgggcgtggtggctcacacctgtaatcccagcactttgggtggccgaggcgggcggatcacgaggtcaggagatcgagaccatcctggctaacacggtgaaaccccgtctctactaaaagtacaaaaaattagccgggtgtggtggcgggtgcctgtagtcccagctactcgggaggctgaggcaggagaatggcgtgaatccgggagggggagtttgcagtgagccgagattgtaccactgcactccagcctgggcgacagagcgagactccgtctcaaaaaaaaaaaaaaaaaaaaaaaaagaaagaaagaaagaaggaaaaaagtcacttgaaaagaatactggactttgtgtccagcttgcatagctgaaaagaataaaaacctgtccacttaaactcattgcaaaaagaagatgtcactcctacaaatagcaaagagtcatgaaattattctatccagaaaagtatacatttcatccctttggataaattttagaagtgaactatgaatacatacggtgaggatagccagctaagaagtcaagaaggatttctcaaatttgctgctcagaaagatcatactctccacaaaacaaataatagcaggctttccaagtcaaccttgaatccagctttcctttatctttccttcttgtgaactttcactagtttactatctaacaatgaatttgacgatagccacataccatcttatagcaatatttgttatcatatcccttgttatttatcattcacctgctctgcttgagccagctacaagtcacatgtcccacgcactttttcctgtttgattttttacagcactttgagacatgtctcattattcctacttgacaggaaagaagccatggaaagttgagtgacttgctcctgatcacaaatgctggccaaggaagagtcgagtttcaaatctaatgatctttccactgcactctagattcctcattttgaactatttttttattttttgcactatagacttttttccacattttgaactgttttttattttttgcactatagacttttctcttatacccaactatattgatgacttcttttaggctagaaacttgtttcacttactttccctttcttcagattgctgcaatattggccaacatgtattgggtacttactgagtcaagtactgtgattgtgccaagtatcttataggaggattatcatcctcatttttacaggtgagaaaggaaaggaggtaaagtcacacacagccaacaaaaatggtagcaccaggatttgaaacaaatcagtctgacccaagttgactttgttaaccactgtatgcacagtcttcttagacatagtaagagctctaattgtgtttggtgatttgattattatgacaaagtaagtaagggaagcagggagaattataagaaataaggctccacaacacttggctatagcaaagccccttaaaacttcaaaaggtcacccaaagaataaagatcaggctgggagcagtggctcacgcctgtaatcccagcactttgggaggccgaggtgggtggatcacctgagttcaggagttcgagaccagcctggacaacatggtgaaaccctgtctctactaaaaatacaaaaattagctggatgtggtggttgccgcctgtaatcccagctacttgggaggctgaggcagggagaatcgcttgaacccaggaggtggaggttgcagtgagccgagatcatgccactgcactccagcctgggcaacaagagcaaaaaactctgactcaaaaaaataaataaatcaatcaataaaataaagatcaatttggagaaattaatgcttattaataagcaatgtcttgcacagcacttcagtttctcaatacattacctaactcaatccttacaacaacaccctatccccattttgtggataaataaactcatgttcagaaggttgaataaattatctaaggttaatagttcctgacctagagctcaaatcttcagtttctatcatattcttgcccttaccctggggtagctaacattcactcactagtattggagctaaaataagggagagaacatataaatgaatacaaaggagacattcacctgccttctctttctccttacatagagaaggttgattatctgctattgtgaagtttgccttttgaaggatagaaatgagaagactttcttaaattttgcctctacgccaagaaattagagtggtaccaccagtagttccattttcaaactatcactgtagctaaagctatgtggtaagggccaaggaaaagaagtattcttgcacttcaaaatgcactgaaataccagtcagtagcataatataaaggaatttagtggagagaagagttgacctcaatctggctccaacatctcggctcttaacccctaccctacacttgttcttcatggggaagctaattgggccactggaagattcagcagctaccatttgcagctgagggacagcccctccctgcttagcaaccaatggatatgcatttatggaacacctgctaactgcgacacacactcctatgtatgagggaaaatacaaaaaatgttaaaggagatgccttcccttgccctcaggaaacttaagtatagttgcaaagaaatgattagcagcaaacgaaaccatggagaagtaagggctaaggtctgtgaaacaagcctagaaaataaccttgtccttgaaaaacacaaaaagaaagaaagaaagaaaagaaactccaaggcccttgtgaaggaaaccattaagtttgcttcacttctgtgtttaggaagacacaaacccagtcttaatgaacctcaaggccacaactactggagacatttaggaattgtcaccacattctaatgtatatatcctctgtttggcccttcctattaatattttgtaaaatttttgaagatatgagcaatgtttaaaaccatgaatccccctttttttataagtaatatttaggctgaataaacaagagaaaataggacataaaggggagccaacgtgtgccttcatttataatgtattcccaagttgtgagtttggtttatcagcaatttatcatgccaaattccaagtcatatttatctatgcagatcaaacacttgattctatttttgccttaatttttttattgggtatgtttatgaccaagtcatatggtattttctgtgacagataaaatgcacaggttattccaatctggctcagccagtcatagcaacatgtagtccttctcatgtcttaagaatgagtatcaagaattcaaagggagttccagatggcatccaaaaagcttacagtttatgcatcacttattctaacagtagaaaaagaatatttgaagccaaaaatagaccttgcatgtagcatgtggaagagtagaaattgccctgatagttaaacaatttgaaattcaagacattaatttctttatgaagcatttgtcacatcataggtaatattttatgcctatcatatatatacttattatgaaatacaaagaaattattcattctatctaagactttgtatcctttaccaatatctctccattctcccacctccaccctagcccctggaaaccacccttctactctctgcttctatgagttcttttttagtgagatcatgcagtatttgtctttctgttcctgtcttatttcacttgacataatgtccttcaggcttatccatgttgtcacaaatgacagaatttccttcttaaggctgaatagtattccattgtgtgtatgtagcacattttctttattaattcatttgttgatggatactcatattgattccatatcttgggtcttgtgaataatgatgcagtgaacataggagtgcagatatctttttgacatactgattccactttgatgggatatatacccagtagtgggactgctggatcatctagtagttttatttttttttattttttattttttttattttgagacagagccttgctatgtcgcccaggctggagtacagtggtgccatctaggctcactgcaatctctgcctcctgggttcaagcaattttcctgcctcagcctcctgagtagctgggattacaggcacgcaccaccatgcccggctaatttttgtatgtttagtagagacggggtttcaccatgtctcgaactcctgtcttcaagtgatccgtccacctcagactcccaaagtgctgcgattacaggtgtgagccaccacgcctggcctagtagttctgtttttaattttttgaggagcctccatactgctttccataatggctctaggaatttacattccaccagcagtgcacaaggattgcttttctccacattctggctaaccagtctcctgtctttttgagaacagacatttcaacacgtgtgagataatatctcattgtggttttgatttgcatttccctgatgattagtgatcttgtgccttttttcatataactgctggacattaatatgccttcctttgagaactgtgtatacaggagaaaataatcacttctcagaggagctttcatttcaaaatatccgggaaaaaaatagaaaaaatggaaaatttatcctagagtaagttgtcttttatattttgaccctgtttgtgacataaactggatgatacaaaactggaatgcaaaggctttaggaggattacttacttacttgtatattgctttaggttgtttgcagaaaattatactaattgaagttcaggctatgatgtgataaaatctatgtcaggagatgagtctacatgcaaagtttgaggaagtgacatttgagtttcaaaacaaaaaagcaattttcaatgtcatatctaggttaacccaaaagatttctttcaccctatttagctgcctctaagatggatgctgaggataattacactgtagaacaataggacgatgcttcacactcacctcacaggctctgttattcccacatactgccagagatactccaaaataaaatcactgcaacatcaggcagttataaacctcaacggtattattttctatttatatacagtatattttatattttacaagtataaaatagaatatatttattctattctctttgacacaaagtgaccataagacatattacttaagtatgactagcaaagtcatggggcttgtcattcaggaggaaactcttaactaactgttcagtttttgttcactgcaccatttacataagccaaactaatgcttcacactgtgcaaaacaatgcacagtgttgtgaatgaatggctaaaataaaactctaatgagtggggtttgaaaaatgcaactttagaaaactgttgagaaaatgttgcacactgcgcattttacaaaatttcgttgaaggacactggatattctttttaggattatggagggaagcaaaattttggctcctacatgcagtttttgtggcctttgcctgaaatagtcatctcccattaattatttagatatcattcatttcctaagacaacatttagggagactgccttaagtacaatttgtacactacccagataagaattctttttggtgaaacatcgataaatattacttggcagtaacaccaagttaaaatatttgtttcacagtcgacgttaataactattatagataaagtgaattttataagacatactcagatctaaaacagcaatatggagctcttcaaatccattgaaacttcataccagcctacggaagtagaggtttttatgcaaactcttcaagaaatatgctctgaacttttaattccttagattgatagaggaattaaatcatgatataactaataggtttgtggtacaaattgctgctgcttaatctgactctgtgtcttcccagtgttctatatgaattagatattccattatctaaagacaatcaaccccatcccacggtgatagctctaggactccctttgagttcattaaatctgtattctcagtctccaaacttctggttaattcaaacagaaaagtcaactggcccatgaactaaaataaagtcatctgaattttttttttattttgcagtgtgataaaagtctcgcactttttatttctgaaagtttctgctttcactgagagcataataggctatccacccttatgcaatcttacatacaaagtcatagtcaggctaaattcaaaaacacatgtgagatagaagtcaacgtttattttctggagaaaagccacacattacaacaaagtgaacaatgaagctggcatccttatcactggtgaccaaaacatttgtgactctggacattggccccacaaatgcgataaacattctgcataggaagtgagttttgctaattaaaaatggatccaaaatactttctactcttcagccaagaattaaaaagtaatagggaggaattgaaatcacttgggtgctacattgagccattctggagaagcaattcagagaatgtcatggcagcctcaaattgctgctcaggagcatcccagcttagaagattgcaggaaaggaagagcaaagtcattcttacatgagaactgtccttaaccagatgaatagactctccattttttaccctggctttgtctcatttaagtcccaaccaatctagctatcattttaggttttactacctgctagtatttaggagcttagggggataaaaaaatccctcaatactcagaattagacttggtgataaaaatcttgacacataaacagaataaagcgctttcattactcctctaaaccacagtgtcatttggtctctatcaaggactgtaagaatttctttcatcaggggaaagaaaaaaaggacaagagcctgcaagatgtagcggaactctcattaaacacagcaggagctttaactggaatccagagtaaggtgaggtaccaggttacaacaatttactgcttttattacaattttgatcacaaggactgattcatgtcatctagtttcttttccttgtcactatcactggtgctaagaatacatcaaattgaaatttaagagcctcatatgtttctgtataacccagtgatgggttgtactgctttgaccttcttaaatgtccctttatttcatttgatatccattcccatagaaaaactataatgctttggttggtcaaaatattaatctttcaaaacctccctggcttagaaaaccaaatttttgtagagagagatgggtagaatctaattttattctaaagcaattagcattacatcatcacagcagaaatatctagaatattacctcatgtcagtgatcttctgatatgttaaaaagggtattttaaaatctgagttatttctttttctttttaaagttacatcattaattacatactcatcaaccaaaatattttatgctccaaatttgaaccgatatagtatgtaagaagtgttcaaaatgaaattattttggtctattttgtctttgaagaagatcacagggatggacctcccaaaaggatttttaaatgggattacatatctgacttttaaaaaaaattatctgaccttgagttatagtgccccaaagtaagcaaagttccaaacacacagtatcatcagaattgagttaaaattatcaccaggggcttaatttctgaaattaaaaaggaaatgttatttccttatgaaaagaaaaggaaccaaaaatgaacttcaaggtagctgatttctgtctatgttaagacttaggtaatgggagaaagggaaaaggaaggacagaattaggagaggagcagtgtttaacaattgcgggtgcaagactcaagttttttagaatccattagcagagaaccctatttctcccattaactgctgtccttttaaatcctggcaccagctctgaggactgcagggtccatagctagtgccccactctacccagtttaaagacaccactgcctggaaatgacaggggtttttttcttaaggaaagaggtgctttctgccacgtatatataaattggtaagcttcaaataaagtgcttttgtcctttctgtctatcagaaactgtgcaaatcgaattgctgtaaaaccaagggcaagagacatcaatcctgcattctatagcatctgattttatcctttatccccaggcacatttcaaaaggaaaaaaatgaggttgcatttaaattgagtatttgggacttgccaggaaaacctcccgctagactaatatgattgcagggaaaacaagagaaaggaaaagtggagagggagtgtgctaacagatcctgggcctcgtcagcagagccgtcctgagcacaaggccatggtcagacatctggtcccgcgaatgacgttttctttatggtcattaagaacaccagtgtgtcgggacacaaacaagtattcctttcagggattatgacacattttctcccaaagtagtatattaatgacatttccagagcattctttactatcttttatatgtgatcaggaagactaatacatatcactacttcttttacacacagcattagccaaaactaaagtgtcaaatacaattttgcctaggatgaataaacagaagaaatttttatgatactgcactatcaattccaaattaaataacaacaaaatgataagtgttaaaattcatattaatgattgttcccacacaagccggaaaaaatctttctaagaagtctttcatgagttaatcccatctttcaaagtgttcagtggctccgaattcagttactgtttcctatcagttcttctttcattaagtctcttcccttttttttctctttgcactatttcccttagccgggtacataatctgctgtgctttattcatttgtgtcttaagtttgtttcccgatgacatacctttccagcaacgccatctggggagtttgggcaactgtaccacgttaggaggaaacccttcttcacaggagagtgtgcctttgctgcagggaaggaattaggatttgcttggactgtggttgcagctggcttttaaggatctccttagaatgcaagcaactcatcaatgagaatctctgcaatggttgtcactgggtagagtcatgctatgtggggtcatagcctttgaaacaaataacagtaaagataaaaatgctattaaaggaatcaccacccacagaggttaactgggttttgtccccagaccacctcgaacaagaaagaacatttttatcagtcattttcttagttttagctgataaaacaaagtaccatagactaggtggcttataaacaacagaaatttatttttcacagctttggaaactggaagtctgagatcaggccgccagaatgatcagattctagttagggcctactttgcttttgcagactgccaacttctagctgcattttcatgtggcaaaaggagattgagctagctctctggtctcttcttataaggacactaatcccattcatgaaggcttcaccttcatcatctaattactctccaaagaccccacctccaaatactatcacattgggaattagatttcaaatacaaattttgcggggacacaaatattcagtccataatagtaatgattactcattatacatagggctctaaatgtgctagcttctgatagtttttacactcacttctctttattagcttgtcaagcataattagggcagtggccttactgaaaattattgaatttagtttcctaaggacagatattgaggagttttttcttcactaaaaattcacgttccgatacagctttcatctgttactactttgtgagatggaaaatcttttattttatttttatgtttggattgacccttcttaataaagtcggcatgtaatatgcttcatgtgtttctaatatgtgcttaattttgcaaaatgttttgcataccagaatgcatttctcttccaaaaaaggtaccagcctacaaaaccttgctgttactgttttcaattagttcatggaattaaatgtattaaatgttttatgctctggcagaaattatgattctcacttaactccatataaatctggatctgcctgggcctttataagtgacacaatttcattaactgaataaacaaatgatacaaagaaatttggtttagccttctaaaattccaaaggcgttcaacaaaatatctcagaatggatgttccaggacttttatggcacaggacaacatgtattgcttattttaagaaaataagctaaatagtgaggggattcttttagcagatcctcaggatgtgttaggttgaatcataggcaaatgatatttgatcattgcacctgttaacacattgaacctcatcctaaaattgtagagctagaagaaagccttctggcagtttttaaatagattgatttactgcaatttatccagaagcttcaccgttgtcactggctacatgtgactttggcctctgtggggctatatcctcatttgtaaaattggtggtgaggtaggtggacagttgactaaataatctcttagaataattctagtatctgtggatctaaagcatccaggggttgaatatgtttctttctggccaagaaaagatgcacctgtcaataatgcccaaactcatcttctgagaatcctctttcccaagatacccactctcccttgggttatattatagtaatgatcagaagcccctgccaagaagaaactgttaacctgggaggtctatattttatttcacagccatctgtttatactttctcacaagttagtgcacagtatacccatcattttctaccattttccttaatttattaattttactaattgcataattaacaaaagtaagaagattttacctccttatccccatctggtagtttgcagatacttggcctgatgacaactgacagtgatgagatactcaccaagtttaccagggcaggaggcttcctagagaaaaaatgagaaaatgaaatggggaaggggagtgaaggattgaggaggtgacaatctggactcttgcaactgcatggcaaggttggcacacaagctgggttgcaacggagggaaggagatccttatcagatgtaatcagagctcagatcgagggctttggtgtgtgtagaaagagggagagacaaagaacttaaaacagagctgccatttgaccttgcaatcccattacttggtgtatacccaaaggagaataaatcattctattaaaaagacacatgtgcttgtatgttcatggcagcactattcacaatagctaagacatggaatcaaactaggtgtccatctatggcagattggataaagaaaatggggtaaatataaagcatgcaatacaacatggccataagaaaaaatgaaatcatgtcctttgctgcaacatggatgcagttgggacccataatcctaagtgaattaacacaggaacagaaaaccaaatacagcatgttctcacttataagtgggagctaaacactgagcacacatggacataaatatgagaacaataaacactgtggactactagaggggggaaggagagaggtttgtaaaactacctatcaggtgctatgctcaatacctgggtgatgggatttacaccccaaacatcagcatcatttaatattcccatgtaaaaagactgcacatataccccttgtatctaaaataaaacttgaaattaaaaaaaaaagaaagaaagaaagaggctggaaatagaggctcacacctgtaatcccagcactttgggtggccaaggtgggtggattgcttgagcccgggaattcaagaccagcctgagaaacctggtgaaactctgtctgtacaaaaaatacaaaaattatccaggcatggtggagcgcacctgtagtcccagctaatggggaggctgaggggggaacatcacttgagcccaggaggtggaggttgcagtgagctgggatcacaccactgcactacagcctgggtaacagagcaactctgtctcaaagagagagaggaaagaaaaaagaaaagatggacagataagaaaatgcacttggagattaagagaaagcagcaacataggaccctggataatgtgtttgcttaataactatcctgatgagttatctgactattcccaaatgagtacgtggcaattcaggctgaaccatcagagtagccctccggaatcttacttatgtacaatagacctgcatgcacatttactagaatgagcctctctctctggtaatcatgtctgcttccactaattccatctgtttcctctctctccctcctatcctgctagatcttaattccttcgaccttcctttgtttttctaactccctttctttctcttgttatttaacctgctatactatgcaattgatctcctctgcactaaggaacatgcacttcagaattctgttgacatcttgcattcctttatatttagtgaaagaatgcaaaggagtctacctggcaatattcactctgcaggaggcaataattattattcaaattaaaggaagcagtaaagagaaattcagaaaaaatgaaatatactaatcttcagcttttcatttcagcctacaaggaaaaaatgaaggagctgtccatgctgtcactgatctgctcttgcttttacccggaacctcgcaacatcaacatctatacttacgatggtgagtaacctaggatagacatacccctgctagctagatcatttggaaaggttgacatatatttgtttcttacagctcctgatataattacatcaatattttgtagctctcactattgacttgccgtgtctagctattatgtccaattgattacctattgctgaaaacagtttgaatttggtgctaataacaacacatcaatgtctgttaagaaatgtggatggattcttattaacagccacatccagcatatcaacatccacaatatgtctaaggtctttctttgcaaataatttaataggctaagccataattggagtagatcataatttgtaagaaaatgctttatacttagaaaactcaagagaaagaatcaacaaccataattgtttttgctttattgtagtctttataaagtttctatactttgtatatacatgtcaaccagctaatgataataataattggctcaataaataaaactgacttacgactgaggccctagataaagagggtctgaaaagaaaagcctaaagaattagcatggcaattaacatgattgaggtgcaactctttaggtttgatttatcctgattcattttgcttactttggctctgccacaatccacatgatcttggtcaaatagatacttggattctctaagtctcatttaactctagcatcttcctcttggagttgttgtgaggtttaaacggtttaatgtaagtcaaatatgcaaaaccaagcctagctcattatatcactctacaatgatagctatcattatcaacatcatccttacctaattcagtcaatttaactaaaatattttatacagttctatgtatcctagatatccctaaggcatattttactaactctcaggctcacaaatatttttcttttccatatatgtaaagaaagacattaatgacaaaacaaactgaccttgtggcagttaaccccttctgcacctttaaagcctattcaaggactcaaaggcatttaccttccaaagttattctatcgtagcacaaaaatcataaatgctaattaactgttccataaggaaatgtcctccatgtgaaaggaattctgtctccaaacaaaacattcattagaatgcagggccaatgcctactttgtacaaattcattcggtcagcaaataaattagacagacctttattatttgctagatgtagctgtgaagaaggatccagctatgtttcttatgagactaatgtcgaactatgggttgtcactgaggatccagagttccatagggcgtagtcctcaccttcaaagaattcagggcttagtagaagagtcttacacaaatgactagaatgtagaacacagagtggttaggacaaaggagccagggatggtttttgctgggttagggaatgaaaaaaggggaagaaaatatgtgaagttatgtgtgagctgattcttgaaataagctgtttttatttgcctgcgttctcttataatccttttccataggcttccataatttttattgagctgtatttaaagttgaatagataattcaacatttctcgtaaactgtgcttcctaaaagagtccgtagagaatttcaaatttctgcagtctttaacttgacctggtatttctatgttagataataacgtgacttgtttattgcaggcaaacattataacaataaattattattattgtttacatttgtaagcactaagtatatggcttgtgctttgcattcagcatcctttatcatttaatcttcacaaccaccttagaaggaaggtactctttttatttccatcttttaaatgaggaaataaaagcataaagaagttaattaacttacctagtgtcacacagctattaagaggggcttactatttggatgcaaatataggcagttctaattccagagcctctaatctaaggcatttaaaaccccatcaccttatcaaataagctgtttttatttgcccgtgttctcttataatccttatccataggtttccataatttttataaaattgtatttaaaatttaagtataatcttggatgccatcaggaaaatgaaaaacatttttacatttgtgaaggaaaaagcccacatcatttccaatatagttattgagttagtattatctagactatctattagcagctaaggatctgaggtcaaggcctgccagcctggcattttacttgaccacaacctccatgtgcactaaccaggctgctaaaagaacattaacgggaacataacctgctggcttggttgccacaattttaaaaagacgttaataaattagagagcacttagaggttaggaaataatatggtggtaaagatctagaaacagtgtcattctggggcacttgaagatgtttagcctgggggaacaacttgaaatggaacataactgttttcaaatacttgaaaaatggtggtgcaccacagagaatggcctaatcatgggtagcttcagacttcaaacaaggatcagtgggctaaaaccagagagatggagtttgggactcaaagaatgctcatctgaaattgagggctgaccagcgaggttcttttaaaaatcattgcattttactaaattgtgagttctgtaattataaatgtcctagcaggtgctagctgtcatcttttctattataaattatactattttatgttataatttgtattatacaggcttaaaacataagggtctgataatctgcttatctttaatacataagccactgatagaaaataagtggctaaccattcttcagttctttttttaattgacaaaaattgtatatgtttgcggtgtatggcatattttgaaatatgtatacattagagaatggctaagtgaagcaaattcacatatgcattacctcacacacctgtcatttatttgtgatgagaacaaaaaatctactctttcagtgattttcaagaatacagtacattgttattaacaatagtcagcatggtgtacaataagtcttctgcggccgggcgtggtggctcacgcctataatcccagcactttgggaggccaaggctggcagatcacgaggtcaggagttcgagaccagcctgaccaacatgctgaaaccttgcctctactaaaaatagaaaaattagctgagtgtggtggtaagcgcctgtagtcccagctactcaggaggctgaggcaggagaattgcttgaacctgggaggcggaggttgcagtgagtcgagatagtgccactgcactccagcctggcaaaagagggaaactccgtctcaataataagtctcttgcatttgttcttcctgtttaactgaaattatgtattctttgatcaacatctccccagtctccacccctaacccctggtaaccacaattctactctgcttccgtgagttcaactttatgaatagtccacatgtaagtgagatcatgtggtatttgtctttctgtgcctagcttatttcacttagcatagtgtcctccaggttcacccatgttgtcaaaaatgacaggatttcccccaacttttttaaggctgaacagtattccatgtgtatgtgtataaattagattagtagatgttgccactccctcctccaccacagtggctctatccctggctcctggctccagccgagtacactagaggaggatattctaaacagcaacaacacaggagcaaagacattacaatggggtgttgtcttattgcccccattagactgtaagcatcttgaagacaaggacccccatcacagagtgatgttgtcatccctggagtgggcactgtgcatgattgatgactggaagcaatgaacatacagaagggcaaaacagaaatcagcaggatgctttgcatttcagcattgactttgccaaatatgcccaactgttcagggagttacattggttctaacgaagctcctgtgattcctaagcacaggaatggtgataatatatataatggtgcatgcatatatacgcatatctagataatgatatctcattatatgtgagaactgaagaactccgttatgtttctcgtctaaccaaaaagggcctacagctacgataatttccaaacaaataaatctgtgctacttgattttcatgcaaagctcatatttgttcaaaaggaaaataaagcttaatttaaaatcaatttaggctatttttatctaagtatgcttaccgttattcaactccctgcagatattgtcaaatttctcaatatggtaaatatttattctgttaaaatatatccatagttacactaaagacagagaggtcttatatgttctaaacaacatagagcaaatgctcataaacagcattttattcctatctcccggaataacaacgctacttccaattgctggaatctaaattattaaaataaacccatgctgcaagctttgtatgcttaacattctcaaatgttcacttttcagatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaaggtaactttttccataggttttccttctctctctccctcccctgctcctccctctcacacactcgggcacacatgcacgcacacacacacacacacacacacacacacacacacacacacacatacagagagcaatgacagctgaacctgtgccatgccaacatgtataggttttcagtagacacagagccaggctagttggggtaaaaactgtaagatagatgctaattttaggctagccaaaccagagctctcagaaatccaaagagcttcagtgctctagtgccccttcccgtatattgaatccccttattataaaagcctcccttccctagaccatcaggcagaagcactgtagagaaaacacagccctggcgaactccagtggtggggaggggaagaagtgctgcttcctccctctcaggatctgtgtcaccccctttgtcaggcgtggttttccttggaattacaaattaccagatcttccctccaagatctttcctgcccagggtaagggccaagagcttgcccctttcctcttcagagtcccactgcctgccctggaagttggtccttccaagatcaggaccttctctgagttctttgaatatgttctttatctttttctaagacttgatggggatttttctctttttgccattggtccctgcttatattaaagagctttccttttgccaaatctttacttttccataatcacatggctaagaagagccaagggtattatttgagaacacttagaaatcctagggactgtgtacacaaacagaagttgtttgaatgtgtctgttccaaccatgtggttatggtagttaatcccatcaaggtactcacgatcatccaaaaatggaattcttttatgtaattcatccccacattgtatttcccaatattttttatgatataattttagaatcaggtaatcactaagaacatgttccctgcacagttttatgatgttttctctaaaaagtcagccaaaactttggacacttctatgttggataattaaaaacagaatgaagataatcctcctcctaaagattgaattctccaagagagaatgcaggacaaacacagatgtgctgtgtatagtatatgtgcatatatacatgcatatatgtacacaaatatgtgtattatcaaataatgaggctcaaacattagaaatccttagattaaattttctaaacaagaaaacactaatctttgtagttgaaaaaaaatcctcctatgatatgtaatatgctgatctcaattttcacctaagagtgatgttctccaaatgtccgatgagcatgtcatatatatatatatgaatttttatatatataattacaatggtaattggtatatagagatatctatattatagatatatatagctatctctatatattacatataccaattatagatataaatataacaatggtaactggtgtatatgtgatgtgtatatatgtatatgtataccataattatatattaatattgtatatatgccataattatatattaatattggtatatatacaccatgattatatattaatattggtgtgtgtatgtgtgtgtgtatatatatatatatatataaaatactagttatcattgttctagatttaaaaaacaggaacctgagctactaactcgactatatatatatatatatatacaggaagttgctttaaaacatttttatcagcttttttattgttatttttagctttattctcatagtaaagctaaaataaattattcaacattatcaaaactttgctgccagcagatgtaagcaatacctaaaacagtggagagcatgttgcacccaaagcagtttaagctctgacccaagcactggcatcttataggcactgggtagagataagagtcataggtcgacatatattgagatgctatgacttgattagaatatggagtcagtgactgaggtgaaattaaaactcaaaccacaattcaacatcctgatttaggatgttgctggtgtttctaggtactacacttaatttgaaagaaattattgaggataaaaaaagaactgggatcaacaaaattaactaggtgttcttataagagtccctgaggttactaattaatgaaactgataaagctcctgcaccctgacagcaagaaattatcaatgattatacatttaaacaattgaattgaactagaaactggccacatggttaaaagacatttacaaatgtaatcatccagtgttatgatgcccagaaaaaaaaaattccttagaatgctttaaaagccgtattccatcacctttccagttatttgttaaacattttgtaatgcaaaaataaccatatagattatgccctagtggtcgggttttatttttagttttttatggtttttttttgttaatggtagagttttaattaaaagaaaatacaactaattagcagaaagtgccaactttaaaaaatcactaattgattttattctattgggttatactgacttaattagcactaatttaaagaactattaattatctttaaagagtctttagcaagtgcatatatctcagtaattatgttagtaaggacatgcctataaccaaaacccaactcaactagttaaaacaaaaagcaaatatgtgactaaaaagtctaggagtggctacagcatcaggaacagctggatccagggatcacagtattatcagaaaactttctttcagtgcctgtcatctcttcctgcatttaactggtttcattatcaagaaagtttaatttcaatagtcagttccaaattatttttctcacaacttagcaactccagcagaaacagagcttctttttcccaatagtttaacaaaagtcccgaaattgagtctcaatggcctggcctggatcacaggcccaacccagaaccaatcattatggccaagaggatgtagtagtttgatatgctagcctgaatcacatgcccaccactgacctgcaaaggattttaggtaagatccctggggtaagaattgtggaggggtagttccccagaagaaaatcgaggtgttctcacaagaggaaggggtaatggatcttaaataaacaaaactatagatgtccacattttctatctataaatgtttagtgttactataacaattagaataattatttagttcatacactattcaatttgtatctcccttctgttgccctgttgccgttattttcttacagatagaatgaaaaatattaatctaggcagctctgtgaaacagtactgtccaaggaatataacgtgagccaggccgggtgtggtggctcatggctataatcccagcactttgggacgccgaggcaggtggatcacctgagatcaggagttcaagaccagcctggccaacatggcaaaaccccatctctactaaaaatacaaaaattcgcagggcatagtggcgagtgcctgtaatcccagctactggggaggctgaggcagaagaattgcttgaacccaggaggtggaggttgcagtgaaccaagatggtaccattgcactccagcctggatgacagagcaagactccatctcaaaaaaaaaaaagaaagaaatgtaatgggagccatatgtgtatttttaaatgttctagaagccacattttttaaaataaaagaaatatgaaatgaattttagtaaaatattcttcacccaatatattcaaaacattatttcaatatgcatgtaatcaatatagaagtattaatgagctgtttcacattattttattcatactaagtgtttgaaatccagtgtgtattttacgtttacaactcatttcaattcatgttagacatattcctagtgcctagtagccaaaggcagccagtagcacagatacggatattaaaacagaaaacacctagtgaataatggggaaattttaggcctaagtttttaaaatccataccagataattattcagattcaaatttactttgttttttcatatatattctttaaaaattacattaatatgggaactcagaaagttcaaaagaaatttccattctatggttttagtctttacattgtcagaactaatgcaagtgtgaagtttaggatgtactgtaagtaataggatcttctaaatctcatgccttcttcagctacctactctgtttctatttcagttcctcactgtggggaggggacttctctgaacctaggtttcatctctcactctcgttcatggtaaacaggttttcctttgtggcacctagcacaattagtaagtaattagtatttactggcatattagtatatatatgcatatgtatttatttaaccctatgtcttctactagattataaactccatgaagatagaacttgtcttttgtttaatagtgcttggcaatagttattactgtaaacattttttttctttcttattcaactcctgttagtcattgcctgagtactacaaatgtttttaagtaaattaataaataataactttcagggccaaatgtgaaagcggcaatatatagcttgttttgattttttattccaccctcccatcctaaaacaattatagtcactaagtttccaaatgacatctgaaattgcactaaggaaatcctagtctgggcaaaatcactcagtcaacagatatttatcaagcacttactatttggcaggccctgttctagacacaggggatactcatcaaacttacattccagtgggggagaaagagctaataaatacatacacagcatattagatgatgcaaaattagcaggacaaagagaactgggggtgtgggggtgaaagaagctaatattatatgttattattactatatataataatataattattggatagtcaaaaaaaaacctcttgaataagacatttgaaaagaagcacaaaggtagcaagggagtagggcgggcagctcttctctgggacctgaacattcaaaatgatgagagcagcaggtgcggaggccctgaaataggaatgtatgaggtgtgtttgagaaataacatggaggccagcgtggctgaagctgagagcagggggagagtggtagcaactgaagtcagaggtcacaattaaggactttgacttcacatgaaatgggagatcatgaaggataataaagccatttcactactttatgtgaatcacagcatctttttaaagaagtatccttttttaaagggggagatgactagaaaaataaatagtgttagataaatagagaaaacaggaaaacattctagactaagacagtgattccagaactaaggatccacagaggcgagaatgcagaaagtgtaggtttcagagcagtgggtagactaagggtttggactagtggatttggatagggagttggagagtagcgaggtgggattagggagggctgtgaatgccaggttagtgtgcaaactccattatataagcagtaaggagtcactacagacttttcaaaaatacatacatgttccacctggcccacgggttagcaacattttcgttgccctggacccatttccttcccaataagttacaggtttgtgaagattctacctagcaaacatattacttttaaataactattaataaattatcttaccatgattataatcaaaggaatctgtaattgctaattatttctgattattaaaagataagcagtattgcactaaattgacataattctaactcaaagtaaatatacagatagacatggctatagatgtgaaatatgatttctgttagggctttttaaatttaaaaaaacttacgagttctcctccctccccctacccttaataccttgaaggcctctttgtgggacttcagggaccccttcagggaactatgacctaggctgtatttggggggctttctgggtttatagctggaaggctgccacagaggcatcgccacttgggctcagattcactttgtgttcaatgttttggcaatgtccccacctccccattccatctgttgacactattgcagcactgaccatctggttactaggttggaggatactccctcgggctcctttgaaccagaattagtgctccagtgattagataatagaagaagcttgtcataaaaagaataagccctttccctgctttttctccattctttgattatcgctggtagtcagtgatgatcatctctatgagtctatatcaatctcatcaggtcagtttgaacctcatctcttgaaatcaaagtttccataatgcaactgacccacaagggtgaaatgacatgaatgctttaaccatccatttatcatttattcattcattcaaccaacatgtatttagcaagaggcagcagagttagcataactatacatcccagttggcccaggacaactccagctaactctcgttgttttgataccattattaattatttctctttactctcataagtgttccactttggacaatcaattacatgagcatccttagcagggcacagtgtttaagggcatctttaaaatattgtctttaagaacatgtggttaagagaatgtctgtgttcaaatcctggttccaccacttaaaagctgtgtgacctcaagcaagtgacttaatctccgtatgtcctcctttgtcaatctgtaaaatgagactagtaatagaacttatggagttagtgtgagaattggaaggttactctacaataaagacatataaccagcatggtaaaagggttagcaattactatgtgaagaagcatccagtttctgacctcacagagattatctagcaaactcatgattttataaagaaaagaagtttctcatcaacagagactgaaatgctaccatacaatatacgttgctttttttttttttttttttttgagacggagtctcgctctgccactcaggctcaggctggagtgcagtgttgccaccttggctaattgcaacctccacctcccaggttcaagcaattctcctgcctcagtctcccaagtagctgggattataggcacccaccaccacacccagctaatttttatatttttagtagagacaaggttttgtcatgttggccagactggtctcaaactcctgacctcaggtgatccacccacctcagccttccgaagtgctggcattacaggcatgagccaccatgcccggccaatatttttaaatattataaaatattctttatcaaattgcatagaagaaaagacagtttgataggtaatagatatataaataggtcaggccaactaaaagtgtcctgaaaaaattaatattgtgaaaacaaaaggattttaatgacattgataaaatctcaccctaaaagagattaaattaaaaatcaccctacttgaaccagttcagtgagatttcattagcatgctctcattactggcataatcagcttcaaagtcactaagcctctgaaaggaagatgtgttgcttattcttaataaaatggcataaaagtagatcattagtcaccaaacatgatagacttaccttttccatttgttggcatctcacattgtagatggcaattaaaatggaatccagggaaagagggggtggtttgtatagcaatggattatgaaacaaagtactggattattcaccgcttgacattcaggaaacattctgctccttacagaatatggcacgtgggccacagaatcttccgtgtgctaccttctcggtgaagaagagcacccccaagtttcttttcctaggagctaaccacagtaaacccattacacactttagcagaagggctcattctaaaggtcttaggattttaatcattttaaatttcctgttatgcttcaggctcttcaacacaaagtgaatattgtactctttggttttacataattatattcaattgtcatatttcaacaggacattatttgtgactttagatgggtcaataatgattttcattgtcagcagtaaagtcaataattacagacacatcacctaccctacttgtgtaaaagcattttttggtactaggagatttagtgtctgatcaacggtcctggatagcaagtaatatatcccccaaataatgaaaagtgacaagaaaataaatatgtttacttcagaaataaatggaaaattagtgctatctaaaatgtagtcttaagtctcatctgtgtacataaagtaaaatgagttttatgtactagttactcaaatttatcttccactccatttgtatagtaattaaactcttacactcagtaatatacaaattggtaattaacctctttgcaaaatgttaaagtgttcctaaatgtacaataagtctcctttcctgtctcattgtttttcgcttcacgtacctctcatgtaattatttcaatgattgagttcagtgtgaggaggtttatgcctagaaaaggtgctcaccaataacgtgcctcagttcccataatagcaagatcgagaaggttctttagtctcccggaacgtcacgttgaacatctcagttctatattttgccttgacatttgcattatatcagctgatcattgtcttgccctaattttcccttttaatattttagtgaccttctatgttaggtacaggttatttagaagtgttcctccaaggccagatactttttccttgaacaatttatttttaacaacttttagcgattttctcacttcaccaccctccgtttcataagtccacgcaatcacaattcctttctgctaatctgcacagtcaagatataaagtaagaatacctatttgaacatgtagtgagaactttacttctctgccaaaaatgaaggaaaatgctgccacttttgtatgtcacatgttttttattctacagcctcactcacttcatgtcatgttttagtgcagttttctggactaactgcttattttctcattgattaaactgcctatttgctcattggaattagagccaatttttttccttgagggtctgactagaagattaaactatgttcatgtgagaatcaatttctacctaagaaatgagttagaggagttatgggcagcaatatctatctggatgctacactgtgaaaaaggaagcgaggttatgcctttctaccccaatggggtagcagagacctcaggaactgaggtagatgcccccctggttattagcgcccctgaataatttgttcaaaaattgactgctggacaggtgtcgtgttgcacgcctgtagtcccagctgtgcaggaggctgaggcaagaggatctcttgagcccaggaatttgaggctatagtaaactaaggtcacaccactatactccagcctgagcaacaaagcaagaccctgtctctaaatttaaaaaaaaatattgaatgcttatgaatagagactaatataggaagtcataagtatttccttgggatagaatgctttccaccataattgacttgacatcctgtatttttgtatgtgtggacttaagttttaaatatttgaaacacagacaattattaagtcctgcaaatgtgtgagttaatagtggatataacattcccttccagggtgtaagaaaaggtaccacagaagtgagcagccctgaagcacagcctggcctagtttggcaggtctctgtgagttagcagcagactcacgtgaccacactctgtactgccttctgtttctgtttcaccccattaattgtgctaaagaaatgcacttgacacctatgctgtgtaatctcatttagccccaatagcaacaaaagtactaaccccattaaattgagtcatttcaaactgagccaaatgttgcactccagtaaatggagtaggcattggttataatgggaattctccattattcataatggaaaccacaggagtttgttcatgcagatcaaatgtgtcccaccaaggcaagaagtatggaaaagtggtgttgctgtattaccttgtaatttcaaagccttcccgtctgaatcttatttccctgctgtttcctcttgactttggttctttcacaaaggaaaattaagaacacaaatataaacattaagttaaaacacaactgaacaaagtgccaaacttaattggagcatctgaaaatgaaacattaggcagttgcagtggcctcttgataataattcacagtaactctctgtaagctgatcctgtctgaagagcagcaggcacaaggcccctggccatgaagtccatctcaaagggccaggctcagcaaagcaggatgcaaacccaggctttccaaataccaggttggggctcatgtcactgtgccacaggagcttctgtagaaaggctacttgaaaaaagtggccattaaaaatccaggtggatcctatctagggcagtgttggaaacactgatctatgggaggaggagcaggaaggaattgtttaaccactgagcagaaatgttacattgctacctgcctttagcagctgtggctgatgggtaccagttgctaagaagagcattacctaacagtgtattaagatagaaaaatgattttaaagcacggcacttagagaatgttgaagttttactttgctttattttgatttgtttggtttgactttgtctcctggagcatcctccatggatttctgttcattacaagagaaacctagggctctaacccaattcctaattcttggacacattgcacccttgttttgtgataatccagccttcttccttgagaaggtttgctggactggaggttacatgtattgaattttctaaaatgaaggtgcaaagctgtctcctcttatttctttgtggtgctcacttcactgtgagatttcctatcaatacagcccaagtcagtgggcatgcatgaggtggagatgagggagttaggaaggacttggactctcatcaaccatcaggatccctgaatccactaactgttcataatcaaagaagtttgaacaaatacttcacacacatgaaattgccaaaattttgcatttgagttgttataccagtaagtccagttgccatcatctccttgtcacaagtgtcttaaattttgcttttgataataatgattaccactcattcagtactaacttacttgatattagacactgcattaaataccttgcaaacattattttgtttgatcctgacaaccatatgagataggtactattcttatccattaccaaaaaaattaatttcatgaagacttttcccagagagagaaactttaaatatttacacacacacctctctccctgtaacaattccgtagtcctgataacagcaaataagcaaagtctgtgtaggatgctttaccaacagtcccacctagaggcaggagagtgaaccagctagaaaatattttattcatatttcttccagaaaggctccattggagtttgaactcaatttatgttataattttcttattatttttgtattggttttcctgaaaccaatacaaagtaagaaagcattggttccactaaaaatgtcctaaaaccagccaagcacagtggctcacacctataatcccagtactttgggaggccgaggcgggtggatcacttaagccaggagttcaagactagcctggccaacatgacgaaaccccatctctactaaaaatacaaaaattagcagggtgtggtagcacacacctgtaatctcagctactcaggaagctgagacatgagaatcgcttgaacctcagaggcagagattacagtgagcagagatcacgccactgtacttctgcctgggtgacagagcgagactctatctaaaaaaaaataaacacataaatagtaaaatgtcctgaaaccattatggggttaaagcaagaggcagggctggttcccaggattttctgtctaatctccagtgagccacagacctattcctgatcaacttgagaataaacacatcagtaaagatgtgtaaggctgtctgactttcccatttctgtagaattttatttgaagagaagtttctcctttctccaggccccatattgtttatacaaaaagacctttccagtaaatgtccacaaccactaccatcaactaaaatgttttcccactaatgctttcaatggtaatcagtatttaacagggcacttaggattattttttgatcaaccattgtttagatattcccacttataattactcctgtgaaggattgcctcggggcatcagctgatcctgagaaattatccagaagccatgagtgtgtaataatttagtcttaaacctaaataggtcagtattgggtgggacttttctcagctgcataatggggagaataaaaagaatatggaaagaagttacgtaacacatcctgggtcacaaacagaggtaagacttgaacacaggcctgacatcaaagcccatgccagtatgacttacaaaaggtagactggactacctgcatttgagtcactagtgatgcttatcactgggcctcaccaaagaaccttggaatcagaatctttggaggtagatgccaggcacctgcattgttatcaagtgctccagtgattaccattcactgtacagagccaaacagactcctgatgctggaagaaaattacagtgctcaaagtgcagggcagggtgtacatctggatctaaatcactgagcaaccacagggtttcaagagagggtcaaaacaaggactttctgctctctgtggccaaggggacactaagtttgcactgttctcagatctccaaagagactttggtgtatgggggatagggaggggggaagggggtgtgaaataaaaggagaaagtgaatttgattatttgattgatgaaaattgaaaagcttattgtagggcctagcctacagttgatgaaaaaacaatggatcaggaagaagatcagaacttgtctcagtcctcaactgttttcctcaggctttggttgaatattgccatcctgtaattcattatagcattttctgttgcataaacgcttagcaacaaagcctttttttaaaaaaatttgtaactcctcaatgaggattaaatgcttcttcttctaagacagtccgaaatatactcacagctgaaaattcagctaaccgcatttcccaactagccacattctatagaaaactctaagccatgcagatgagtacagacttgacaatagtgctcaaggctgggagtactattcatctgaaaagaatgctccctccaattggtgggccgttattctgctaggtttgtgtttggataattataagatggctatgtttttcttccccagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggtaatctcagcagagtcctgagcagatggatatattcatatgcagcacagctgggtgaacttccatatgcctgagcacagagacgaagtcaaaatttgctgcaggtgtgaggacaactaactcccatgggcagggtctcacagtgtagcattgagttagcaggaggtgcaacatggtagagaaatgggaatccatcatgaaagctggaattttgtcaaattttcccatggtgagtggattcagggaggctgattcatgcttttgaaatgtgtaagacttctatacaagcctcacgaggcaatctgtaggaaaaatgttacactggaaatattaatgtctatatattatattgatataagtataaataacatttgatttaatatttgtttaatatatgacattaaatatatatttaattaaaatattaaattagaaaaatatatttgccagaaaaggccagggtatttatgaacactggtaagcccattctagggtataatagcatcacatgggaccatagcaaagattagctcataggggatgtttcatccagttctggtatcctggtgcccttctcttcaacaacctaaacatatattcattcccatgagtcaggaggagctgtgctggagttcttctgaaaaatgctgtctttcacttttgtactctctatgctgtctcccacctatcccctcaaaaaacctttcctttgaaaatatacagtatagctgtgagtagtttagctgtgtccgtttccagaaattggaataagcattgagaaatgggatgtttgagaaagacgcctcaatccttttctgagcagtcagtcacccttcccgccagtagcaagtgcctttgtgtgataggcattggagatgcagagcaaaacaggagtgtgcctgtcatcagagccctgagagtttaattagatgagcctcctgttttctatttctcagagtttcatgtcttctgttagagatggcccttctcatctaaggttcaaaaaaccttatcctgaagttctgatgattctgttttcattctcagtctctgactgcaaatatccaactagaaacaaaggaaatcaggcatgaaaacttttaaagatataattgcatggagatcttcatttgtgctcgtgaggaatttttgaaagcattgctggggaagggtgtgtgggctctgatgcagcagtaagacactgaggctctcagaggtccgtggacgagtactgctgacttgggcaagaaccggaatagttacctgatgccttatccgaaacatgaaagttcggattaaatttgtatttataagctagtgtttttatactctcagaacaatgtcattgcgtttcacccaagtgagtcaagtcacgatttggaagaggcaacagaatttggctctctccaggtgatttatggcggtataggaacacatgttttactcagatacaggggagcaaagttccatttgctaaagtttactcccctgaccttcaaccagtcagtcttcctccatctgccaccactttgcacttctccagagaactaaggatgttcccgcttgaccagtgctcataacatggacagcagagggccactgtgtgatctctttgagatcactgtgactcaaccttcttctcacatcctaggccctaaaacaattaagtgaagttgctaggaacggtacctgctgatcttattgcagcattctcaattaggcctcaatgcaagatttatatcactggcagtcctggagcatttttgtttttcaaattacacatacccaaacacacggcatagcctccttttttgtttgtttgtttttttgagatagagtctcgctgtgtcgcccaggctggagtgcagtggcacgatctcagctcactgcaacctctgcctcctgggttcaagtgattctcatgtctcagcctcccaagtagctgagattacaggcgtataccaccacgcccagctaatttttgtatttttagtagagacagggttttgccgtgttggccaagctggtctcaaactcctgacctcaagtgatccacccacctcggcctcccgaagtgctgggattacaggtgtgagccaccgtgcccagccagggcatatccttcttgatttcaattgtaaaatagttcaaaaattttccatattttatctaatatttccagaagtgctagcttttaacggaccatttttttcctctgtgtgtttttttctcttcacctagcccagccatgctcagctcatttttgtactctttccactcccaaccaaatttagtgccctcccccatacatgcatacatgtacatctgcacaccacttttcctgcaaataatcaacccaaagagtgcttaaaattcctgacatcaacccacagaatctccaaggatgggacccagcatccatacattttaaaaactctccatatagttccaatatgcagccagatttgagaactagtggttcgtagcctgttctgatttaaatctcagctctcagcagtctatcccacgtcacataatgcagcccagagaaattctaggaccacatttttttctggtatttcatagctaatgaggtgcttttcaaatctaataggatctttggccagtgtcagtcaagatcttttatctcctcaataaaaaggaaataccatatttactttgatttgatgtatatcacataggtggatttaatacaaaattgtggtttacatattgtgaatgtgtatactaaaactactttgctttttcctaaaataagacaaagttttatattggaagtaatatttagcattttgtttgaatgaagttactcctattaaattagaaatttaaaagagggtcagtaataacagtaaagccaaaaggcatgacactgccaacgtaacataagctgctctgaaatctaccatatcaaaagataattatgctgggcatggtggctcacacctgtaatcccagcactttgggaggccaaggcaagagaattgcttgaagccaggagttcgagaccagcctgggaaatataatgataccttgcctcaaacaaaaattcaaaaattagccagcagtggtggcacacttgtaaaaatgcctgtagtcatagctacttcagaggctgagatgaaaggattgcttgggccaaggagttcgagactgcactccaacctgggaaatattgtgccactgcactccaacctgggaaacagaacaagaccctgtctctaaaataaaaagaaaaaaaaagatgaccacttctgaaatgacacctatcaatgagttaatcattcaatgaatatgtattgagtccctactatatgcttaggaacctttgtaatatcattaccaaccatgtctttcccaatacagacaatacaaaattcagcaataaataatatagcaccaacaattagagaataagacaacatgtagtatggtccaatatagacagtaaatacaaagacactgaataatatcagtaaaagtaaattcacatcaaggtcactacaccatgcgcccacccttatgatagccctcactggccctatcaattaagcaagagacatgatacaactctgtgcaagcttttccacaatctgcctaccattcagcactcagtcgctcttcccttcaattaagagaattgagcattcaagcatattttcaccatgatgcccataatggtatcttcaatgtcactgactgataaattcccagaaacccctcagagccccagccatgttagctcaaagcctttagctaaaactgaaagcctaaagcaaaagcagccctggctgcacttcggaatctactggacagctctttaagggattctgatttaatgtctggaatagggccaagaaccttgtattattttaaaggctcactagtaggctctaatatttagccgtggttgagaaccactgtgctaaatgtttcttaaatatgctttgtgatgtcatcataaattatattttagtattttttgtctttgttgcataagtgttctttcttcctccaaagaagaatgttacactcatttcttatttcagtttcctgttttcatagcacctcatcttaacactccaggctattatatagaaaagaatcaaatgtggagaaggctgtgggagaagggatgcctgtgccacaaaggcctgcattaggctgacctattgatgtcatatccaggactcaaaagactagtctgtggattatgactggtgaagttcaaaatgttcttattcttagagtggtatgagaagtagaaagagagagaaacagagaaggggaggagaggggaagagaggaagatgagagaaaggaaagagagggggaaacacctgttcttgacatacaggaatgattcaagacattttcttcctcccctgatgtgtccctttctcccctaacgcactatgcagcatcctgcagaaaattcaccacctgacccttttagaaaccctgagtagtaggagcgccaaatgacccaatcaagaattgcagtgagacagttagttttgaaaaatcagttaaagcatgtataatcattttaacaacaatacatctattcactaaacatataattttaatgtcaaatatttacgtgtaaacatattgaccaatctttcgatgtagttgggcccaataccttttccaaaaattgatcagttaatgggggttctatgggggtttcttttcttgccattattcacacttatgtcacattagctatgatttgcagttttaatttctttaaaattgagtagggactaaagacatctccaaaaagcctggatatagactttttacaacttttccatagcttttatagttgactcacccagtatctactaaatacttcactttctcacgtatttccaaaggtttctctccaccctcacaattttccattaatgtagtacttaattaaattagatagttaaattttcaaatgtgaattgctaaacaggtgtggaaataccattggctataatcaagcatataacacaaccatttgagaaggaaagtatgtggcaatattagggaagagccctttcctctcaagcaattcagcatttaggaaccatcagacagcaggacgatggagggaacagagagggttaacatggcaagttactgaagaggacttctactgaatcttgttgaattccccacttaatccagattgtatcatatcttctttcttttgtaattctaccatatcatcttagtcaatgccaagacttctgagctcataacatggtaacaaataccaaaggagctttcagtatcgtttagaaaggagagaagcaagtaacccagacaaacttgacaactgctttcccctatccaaccatgaagtacagtacttaggaaataaaagaaattgcttcactataattcatcatttcacttctaatatctagaaaatgtcaaatgaaaatattatagccatattttagtggcaatagtagcacataatatgatgcaacttaaaatgataaaaatattttcagggaataagattctgtgattctttccctaagaggtaattttgataatatgtacctgttttgtaaatgtcaatagtcttggggatacaggtggtgtttggttacatggaaaagttccttagtggtgatttctgagattttagtgcacccaatacccaagcagtgtacactgtacccaatatgtagtctttcatccctcgcccccactcccaaccttcccccacaagtctctaaagtccattatatcactcttatatctttgcatactcatagcttagctcccacttatgagaacatatgatagttagtgctcaattcctgagttacttcacttagaataatggcctccagctccacccaagttgctgcaaaagacactatttagttcctttttatggctgagtagtattacatggtgtatatataccacattttatttatccacttgttggtcaatggacacttaacattagttccatatctttgtaatttcaagttgtgctgctataagcatgcatgagcctgtgtctttttcatataattacttcttttcctttgggtagatacccagcagtgggattgctggatcaaatgatagttctactttcagttctttatgttttccacagtggtcatactaatttacattcccatcaacagtgtaaagtgttcccttttcatcacacccatgccaacacctattgttttttgactttttaattacggccattcttgcaggagtaaggtggtatttcattgtggttttaatttgcatttccctgatgttgacaatatttaactctttagttatagattccagctattatcaatttacacctattgcattcttctcatcttttgttttcttgtgattctgatgcacaaatatcatttgtgcaaccacttactgttgaacatgtctgatgaacacttactattgaacatgtctgatgaatgaataatgaaataggaaaagggattaaaactagcctttattaattgtttgctataggccagacatttttggatgtactatcacatttcatccaaacaacaacctaaaagaaaatactgtgattatccccatttcacatctaaggaacctggtctttaggaagattaagtcatttggccaagatcacaagtagaccacagagactagatttgaatgcaagtctgtttgactccaaacctttttactatctgcccatgacccctgatcaccaacatctcaatgtatgaacatgtgctttcttagctcacacaactcactcctgaccccttttttatattgcaagtgcatagtcattagtaaaaagaaggatttttgatgatactgacctcatcttgaatttaattaggctcatatgacagaattccatagatggaattgacatcctaggtcatatagtccaagtccttgtttatatttgatacctagtgagattaaagggacattaaaaagtaaagaaaggaaagacctcatatttcttaccttccagtagagaaatctttctatgaaatcagaggaaagaattagaggaccagaatttttcctaaaatcaactttcatacatcttttttcatataaaaggcatagctgcatacaatgctaaaatattgtattacatttcctttatattgatgggaggaagggggtaaattgcagaaaacattgtaaatttagatatgcttgggcctctgacagtgcctagcaaatatcaggagatcaataatgaaataaatattatcaaagagtagtcttcttgatgaaccttctctgagtatcacaactgctttaggaacctctagattcaaggtctagtaattgcaaacagtgagctgataagaaaaacagactgtatgggaaattacatgcttcctgcatgactgccttttgttctcccacattttgatataaagtcacattaacagttcatgagtaaatattcgataatgtgaacgtaaagtgttcaaataatagagtgactaaaatgcctgaaaacaaataatttttaattagaaactcataatcatttattttctctttttccacattatctcaagctcacaaattatatttattctttcctatggcaaaatccattttgttaacactaattttgagtttaacaagaagtgtactccaaagtagcctaataatactaattataatgtttcctgctatgttatcagtttgaatttatatgaatctttagacttgaggcttctttttcctagcatagtgatggtctgggctttttctcaatttttgccagagctcagctctcactaattagtttctttctgcatgagaaaaagattttgcttcatctttttccttataatagcagaacaaaaagaagaatcagctgcatccatgctaatttcccctgtgacatttccaaacaggatttgatttctctatgcatgcctctttccttctcttcatggtttttgaacatatacaaaagctcatttaaaccaattaaataaaattgtttttaatctctttctctagagtcaacttcctgcttactccaactctgtatctttgaaggaagtatagggtggtctatgccttttttctcccagaatctacacttgaaaagacacatttttccatgcaactataaaatgttctcctcactcaacattgaaattgtatagcagtgattaagagagtgagctgtagagccaggttccctgggtttaaatcccacttgttagtatcatgaagatgggcaagttacttacccttcctgtgtttcagtttcttcatctgcaaaatggggacaataatagaatgtccactataagattattgtgaggattaagggaattaatacaggtaaaacgtgtactgatgcaggtctggtacacattaagtgcctaataaatattcagtattatgatataaagaaccctataagtgtagactccttgagattaatagagtttaacgataagttttactttatagctggtcaagtttatttcttctgaactaaaagaatctatagagtctcaatttctggagcttcagagggaaggagagaagcaatgtaagcaacattctacagaaatataaataatactactaataattagcatcttaaaatttcaattcaatgaacatttatttagcgcctatgatatatgcaagacagtttgattttagtcatctgatgtatagccacatactaaaaaatactgattttagtcatctgatgtatagccacatactaaaaaatacttcctccatcagttccctcctcaggaagttcagttcccaatcccaggctagtaccttggttccttatgtaaataaacatccaccaattacatgctatctgcaaagcactctgctaggccctgcaaatggaaaaaaaaatgataaaacatagtccaggccctcaatgagcttacagtcaaatataatagaggagacaagaacagagaggctcataatacaactagaataaaatgactgccgaataaaaggaaagatttatgcaggtgttcaaatggaaagtgagataagtttgcaggttagtctttgcagtctcataaaaatctttatggagaaaaggacaatggtcatagggcttaaagagtaagtttataatcctgaccagtggagatgaaagactagcattgaaaattgcatgacaagacaattccattaaactgaaacatcaagtgtgtgtaggaaaagatgggggttatgactggaaacgtcacttggactgcaattatgaagggccttgacaaacaggtcaagagtttaagaagcagtatagaaagtcttcgtcctggatctagccctcccagagtgtccatcaggattataaagtccttaaaatattagtcaaaaggaacgacatcattagaaatgatagagaaacaataatgtgatgttttattacctttctctggatttatactctgatcctaatattcaaaactatcttaataacatgaacttttggtcatagttttaaacaaaaacagtgttaaatatattttttaaaacacagtaagtcttgtaagatcttttctaacatgacattttgcagggcccatattttccttctgaaatgggaaaaattcataaaagtagacaccaaactgggttacttctagtcaagcgcatggtacgcaaaggaccagacaaaaagggcctgtgacatttcttcttccttttgtgttttttaggagaggcatgctgcggaggtgcgcaggaacaaggaactccaggttgaactgtctggctgaagcaagggagggtctggcacgccccaccaatagtaaatccccctgcctatattataatggatcatgcgatatcaggatggggaatgtatgacatggtttaaaaagaactcattataaaaaaaaaaaaacaaaaaaaatcaaaaattaaaaaaaatcaatgcggtctctttgcagaatgttttgcttgatgtttaaaaaataccttggatcttattttgtaaatacttacatttttgttaaaaaatacaagtattgcattatgcaagttatttcataatcttacatgtcctgtaacaggcttttgatgttgtgtctttccactcaaatgaatttgctaggtctgttctttttgaagctccccatgtctaactccattccaaaagaaaaatgaggtcagtagacagtctatggtgctagaaacccaccattgcctaatgacctagaaggctttgttgtctctgagcttgactaagaccatacctagatcacaggtattatgactccacatgaaccttcacatttgttcgctcataatctacttactgcctaaaaactacaaaaccaggctaagaaataccaccagtcatagcatttacttctgcttctcctggattatgtgctacaaatgtgctttggctttagaaagggatggatgagaagacagacctgagaccaatctgggtagaagcaaaaagttgaaccttttaaagtgctgaacacaaatccaaattcgaatggttcaagcagccgtgaaatcgctcttcataaagtgggcttaattctctagtttaagttcttttgatggaatgaattaattaatgtgtcaggtggcttatttgtggatgccatgattgatgatgttcattttaagctcttacctatagtacaagtacatgatgctactgaatatttttccacttggaaactgtgagctggttgttgcattaaaacacacatacaaacaaaatcaaaaacactgcggactttcactcaagctggtctttcttccccagtgtaaggcaatcctgcctactaacaacaccaacaacaaaacactccatctgtgaagctgacgcagttaagggggctaggcagggcatttgtgccaactaagaatcaccagatacccaccataagtacctatcgcagttttgaagtcgtttctccccaactcccaactcctgaaggttgctgcctgcatatttactcttcattagtgctattttcctgtatgtcattgtgagcaagctgtgattaataaagaattggagttctgtgaactaataaaggtttggtctgtt

SEQ ID NO: 1392 - RNA, 23 nt, Artificial Sequence
  Description of Artificial Sequence Synthetic oligonucleotide
auccaauuaagagagagugaugg

SEQ ID NO: 1393 - RNA, 23 nt, Artificial Sequence
  Description of Artificial Sequence Synthetic oligonucleotide
aauccaauuaagagagagugaug

SEQ ID NO: 1394 - RNA, 23 nt, Artificial Sequence
  Description of Artificial Sequence Synthetic oligonucleotide
uccaauuaagagagagugauggg

SEQ ID NO: 1395 - RNA, 21 nt, Artificial Sequence
  Description of Artificial Sequence Synthetic oligonucleotide
uccaauuaagagagagugaug

SEQ ID NO: 1396 - RNA, 19 nt, Artificial Sequence
  Description of Artificial Sequence Synthetic oligonucleotide
ccaauuaagagagagugau

SEQ ID NO: 1397 - RNA, 21 nt, Artificial Sequence
  Description of Artificial Sequence Synthetic oligonucleotide
aauccaauuaagagagaguga

SEQ ID NO: 1398 - RNA, 21 nt, Artificial Sequence
  Description of Artificial Sequence Synthetic oligonucleotide
caauuaagagagagugauggg

SEQ ID NO: 1399 - RNA, 23 nt, Artificial Sequence
  Description of Artificial Sequence Synthetic oligonucleotide
gaguccugcaauaugaauauaau

SEQ ID NO: 1400 - RNA, 23 nt, Artificial Sequence
  Description of Artificial Sequence Synthetic oligonucleotide
guccugcaauaugaauauaauuu

SEQ ID NO: 1401 - RNA, 21 nt, Artificial Sequence
  Description of Artificial Sequence Synthetic oligonucleotide
guccugcaauaugaauauaau

SEQ ID NO: 1402 - RNA, 21 nt, Artificial Sequence
  Description of Artificial Sequence Synthetic oligonucleotide
gaguccugcaauaugaauaua

SEQ ID NO: 1403 - RNA, 21 nt, Artificial Sequence
  Description of Artificial Sequence Synthetic oligonucleotide
ccugcaauaugaauauaauuu

SEQ ID NO: 1404 - RNA, 19 nt, Artificial Sequence
  Description of Artificial Sequence Synthetic oligonucleotide
gaguccugcaauaugaaua

SEQ ID NO: 1405 - RNA, 19 nt, Artificial Sequence
  Description of Artificial Sequence Synthetic oligonucleotide
ugcaauaugaauauaauuu

SEQ ID NO: 1406 - RNA, 23 nt, Artificial Sequence
  Description of Artificial Sequence Synthetic oligonucleotide
gucuucugccgaguccugcaaua

SEQ ID NO: 1407 - RNA, 21 nt, Artificial Sequence
  Description of Artificial Sequence Synthetic oligonucleotide
aggucuucugccgaguccugc

SEQ ID NO: 1408 - RNA, 21 nt, Artificial Sequence
  Description of Artificial Sequence Synthetic oligonucleotide
cuucugccgaguccugcaaua

SEQ ID NO: 1409 - RNA, 19 nt, Artificial Sequence
  Description of Artificial Sequence Synthetic oligonucleotide
ucugccgaguccugcaaua

SEQ ID NO: 1410 - RNA, 23 nt, Artificial Sequence
  Description of Artificial Sequence Synthetic oligonucleotide
gcacacaugcucacacagagagc

SEQ ID NO: 1411 - RNA, 23 nt, Artificial Sequence
  Description of Artificial Sequence Synthetic oligonucleotide
acacaugcucacacagagagcca

SEQ ID NO: 1412 - RNA, 21 nt, Artificial Sequence
  Description of Artificial Sequence Synthetic oligonucleotide
acacaugcucacacagagagc

SEQ ID NO: 1413 - RNA, 21 nt, Artificial Sequence
  Description of Artificial Sequence Synthetic oligonucleotide
gcacacaugcucacacagaga

SEQ ID NO: 1414 - RNA, 21 nt, Artificial Sequence
  Description of Artificial Sequence Synthetic oligonucleotide
acaugcucacacagagagcca

SEQ ID NO: 1415 - RNA, 19 nt, Artificial Sequence
  Description of Artificial Sequence Synthetic oligonucleotide
gcacacaugcucacacaga

SEQ ID NO: 1416 - RNA, 19 nt, Artificial Sequence
  Description of Artificial Sequence Synthetic oligonucleotide
augcucacacagagagcca

SEQ ID NO: 1417 - DNA, 25 bp, Artificial Sequence
  Description of Artificial Sequence Synthetic oligonucleotide
gagtcctgcaatatgaatataattt

SEQ ID NO: 1418 - DNA, 25 bp, Artificial Sequence
  Description of Artificial Sequence Synthetic oligonucleotide
gagtcctgcaatatgaatataattt

SEQ ID NO: 1419 - DNA, 25 bp, Artificial Sequence
  Description of Artificial Sequence Synthetic oligonucleotide
aatccaattaagagagagtgatggg

SEQ ID NO: 1420 - DNA, 25 bp, Artificial Sequence
  Description of Artificial Sequence Synthetic oligonucleotide
aatccaattaagagagagtgatggg

SEQ ID NO: 1421 - DNA, 25 bp, Artificial Sequence
  Description of Artificial Sequence Synthetic oligonucleotide
aggtcttctgccgagtcctgcaata

SEQ ID NO: 1422 - DNA, 25 bp, Artificial Sequence
  Description of Artificial Sequence Synthetic oligonucleotide
aggtcttctgccgagtcctgcaata

SEQ ID NO: 1423 - DNA, 25 bp, Artificial Sequence
  Description of Artificial Sequence Synthetic oligonucleotide
gcacacatgctcacacagagagcca

SEQ ID NO: 1424 - DNA, 25 bp, Artificial Sequence
  Description of Artificial Sequence Synthetic oligonucleotide
gcacacatgctcacacagagagcca

SEQ ID NO: 1425 - RNA, 25 nt, Artificial Sequence
  Description of Artificial Sequence Synthetic oligonucleotide
gaguccugcaauaugaauauaauuu

SEQ ID NO: 1426 - RNA, 25 nt, Artificial Sequence
  Description of Artificial Sequence Synthetic oligonucleotide
gaguccugcaauaugaauauaauuu

SEQ ID NO: 1427 - RNA, 25 nt, Artificial Sequence
  Description of Artificial Sequence Synthetic oligonucleotide
aauccaauuaagagagagugauggg

SEQ ID NO: 1428 - RNA, 25 nt, Artificial Sequence
  Description of Artificial Sequence Synthetic oligonucleotide
aauccaauuaagagagagugauggg

SEQ ID NO: 1429 - RNA, 25 nt, Artificial Sequence
  Description of Artificial Sequence Synthetic oligonucleotide
aggucuucugccgaguccugcaaua

SEQ ID NO: 1430 - RNA, 25 nt, Artificial Sequence
  Description of Artificial Sequence Synthetic oligonucleotide
aggucuucugccgaguccugcaaua

SEQ ID NO: 1431 - RNA, 25 nt, Artificial Sequence
  Description of Artificial Sequence Synthetic oligonucleotide
gcacacaugcucacacagagagcca

SEQ ID NO: 1432 - RNA, 25 nt, Artificial Sequence
  Description of Artificial Sequence Synthetic oligonucleotide
gcacacaugcucacacagagagcca

SEQ ID NO: 1433 - DNA, 564 bp, Homo sapiens
atggctaaaacagcaatggcctacaaggaaaaaatgaaggagctgtccatgctgtcactgatctgctcttgcttttacccggaacctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaagctggtcaagtttatttcttctgaactaaaagaatctatagagtctcaatttctggagcttcagagggaaggagagaagcaatga

SEQ ID NO: 1434 - protein, 187 aa, Homo sapiens
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKLVKFISSELKESIESQFLELQREGEKQ

SEQ ID NO: 1435 - DNA, 540 bp, Homo sapiens
atggctaaaacagcaatggcctacaaggaaaaaatgaaggagctgtccatgctgtcactgatctgctcttgcttttacccggaacctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggagaggcatgctgcggaggtgcgcaggaacaaggaactccaggttgaactgtctggctga

SEQ ID NO: 1436 - protein, 179 aa, Homo sapiens
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKERHAAEVRRNKELQVELSG

SEQ ID NO: 1437 - DNA, 525 bp, Homo sapiens
atggcctacaaggaaaaaatgaaggagctgtccatgctgtcactgatctgctcttgcttttacccggaacctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaaggagaggcatgctgcggaggtgcgcaggaacaaggaactccaggttgaactgtctggctga

SEQ ID NO: 1438 - protein, 174 aa, Homo sapiens
MAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKERHAAEVRRNKELQVELSG

SEQ ID NO: 1439 - RNA, 19 nt, Homo sapiens
gcucaagcauggauucuaa

SEQ ID NO: 1440 - RNA, 19 nt, Homo sapiens
caaucaagguaguaauaug

SEQ ID NO: 1441 - RNA, 19 nt, Homo sapiens
gggcuucgcuacaggaauc

SEQ ID NO: 1442 - RNA, 19 nt, Homo sapiens
caggguggauuugguaaua

SEQ ID NO: 1443 - DNA, 20 bp, Artificial Sequence
  Description of Artificial Sequence Synthetic oligonucleotide
  Description of Combined DNA/RNA Molecule Synthetic oligonucleotide
aaggcttcatattgtacttt

SEQ ID NO: 1444 - DNA, 24 bp, Artificial Sequence
  Description of Artificial Sequence Synthetic primer
ctcagtgccttattcagtcttctc

SEQ ID NO: 1445 - DNA, 20 bp, Artificial Sequence
  Description of Artificial Sequence Synthetic primer
tcttctgccgagtcccattt

SEQ ID NO: 1446 - DNA, 24 bp, Artificial Sequence
  Description of Artificial Sequence Synthetic probe
tcagcgtctgcacatccctacaat

SEQ ID NO: 1447 - DNA, 21 bp, Artificial Sequence
  Description of Artificial Sequence Synthetic primer
ccacgaactttagcttctcca

SEQ ID NO: 1448 - DNA, 20 bp, Artificial Sequence
  Description of Artificial Sequence Synthetic primer
gccaattgtttcagcacctg

SEQ ID NO: 1449 - DNA, 26 bp, Artificial Sequence
  Description of Artificial Sequence Synthetic probe
actttcttctttcctctgcagcctcc

(19) United States
(12) Patent Application Publication
(10) Pub. No.: US 2022/0333105 A1
(43) Pub. Date: Oct. 20, 2022

(54) OLIGONUCLEOTIDES AND METHODS OF USE FOR TREATING NEUROLOGICAL DISEASES

(71) Applicant: QURALIS CORPORATION, Cambridge, MA (US)

(72) Inventors: Sandra Hinckley, Sudbury, MA (US); Duncan Brown, Berkeley, CA (US); Sudhir Agrawal, Shrewsbury, MA (US)

(21) Appl. No.: 17/616,350

(22) PCT Filed: Jun. 3, 2020
(86) PCT No.: PCT/US2020/035811

§ 371 Date: Dec. 3, 2021

Related U.S. Application Data
(60) Provisional application No. 62/856,264, filed on Jun. 3, 2019.
(60) Provisional application No. 62/914,252, filed on Oct. 11, 2019.
(60) Provisional application No. 62/949,817, filed on Dec. 18, 2019.

(51) Int. Cl. C12N 15/113; A61K 9/00; A61P 25/00

(52) U.S. Cl. CPC C12N 15/113; A61K 9/0053; A61P 25/00; C12N 2310/11; C12N 2310/315; C12N 2310/346

(57) ABSTRACT

Disclosed herein are antisense oligonucleotide sequences, and methods of use for treating neurological diseases. Described herein are oligonucleotide inhibitors. In various embodiments, the oligonucleotide targets a transcript for the treatment of neurological diseases, including motor neuron diseases, and/or neuropathies. For example, inhibitors of the transcript can be used to treat PD, ALS, FTD, and ALS with FTD.

### CROSS REFERENCE

This application claims the benefit of and priority to U.S. Provisional Patent Application No. 62/856,264 filed Jun. 3, 2019; U.S. Provisional Patent Application No. 62/914,252 filed on Oct. 11, 2019; and U.S. Provisional Patent Application No. 62/949,817 filed on Dec. 18, 2019, the entire disclosure of each of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on May 29, 2020, is named QRL-002WO_SL.txt and is 378,978 bytes in size.

### FIELD OF THE INVENTION

The present application relates to inhibitors of STMN2 transcripts that include a cryptic exon, including STMN2 antisense oligonucleotide sequences, and methods for treating neurological diseases.

### BACKGROUND

Motor neuron diseases are a class of neurological diseases that result in the degeneration and death of motor neurons—those neurons which coordinate voluntary movement of muscles by the brain. Motor neuron diseases may be sporadic or inherited, and may affect upper motor neurons and/or lower motor neurons. Motor neuron diseases include amyotrophic lateral sclerosis, progressive bulbar palsy, pseudobulbar palsy, primary lateral sclerosis, progressive muscular atrophy, spinal muscular atrophy, and post-polio syndrome.

Amyotrophic lateral sclerosis (ALS) is a group of motor neuron diseases affecting about 15,000 individuals in the United States of America. ALS is characterized by degeneration and death of upper and lower motor neurons, resulting in loss of voluntary muscle control. Motor neuron death is accompanied by muscle fasciculation and atrophy. Early symptoms of ALS include muscle cramps, muscle spasticity, muscle weakness (for example, affecting an arm, a leg, neck, or diaphragm), slurred and nasal speech, and difficulty chewing or swallowing. Loss of strength and control over movements, including those necessary for speech, eating, and breathing, eventually occur. Disease progression may be accompanied by weight loss, malnourishment, anxiety, depression, increased risk of pneumonia, muscle cramps, neuropathy, and possibly dementia. Most individuals diagnosed with ALS die of respiratory failure within five years of the first appearance of symptoms. Currently, there is no effective treatment for ALS.

ALS occurs in individuals of all ages, but is most common in individuals between 55 to 75 years of age, with a slightly higher incidence in males. ALS can be characterized as sporadic or familial. Sporadic ALS appears to occur at random and accounts for more than 90% of all incidences of ALS. Familial ALS accounts for 5-10% of all incidences of ALS.

FTD refers to a spectrum of progressive neurodegenerative diseases caused by loss of neurons in frontal and temporal lobes of the brain. FTD is characterized by changes in behavior and personality, and language dysfunction. Forms of FTD include behavioral variant FTD (bvFTD), semantic variant primary progressive aphasia (svPPA), and nonfluent variant primary progressive aphasia (nfvPPA). ALS with FTD is characterized by symptoms associated with FTD, along with symptoms of ALS such as muscle weakness, atrophy, fasciculation, spasticity, speech impairment (dysarthia), and inability to swallow (dysphagia). Individuals usually succumb to FTD within 5 to 10 years, while ALS with FTD often results in death within 2 to 3 years of the first disease symptoms appearing.

Like ALS, there is no known cure for FTD, or ALS with FTD, nor a therapeutic known to prevent or retard either disease's progression.

Thus, there is a pressing need to identify compounds capable of preventing, ameliorating, and treating neurological diseases such as amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, corticobasal degeneration (CBD) and/or neuropathies such a chemotherapy induced neuropathy.

RNA-binding protein transactive response DNA-binding protein 43 (TDP-43) is involved in fundamental RNA processing activities including RNA transcription, splicing, and transport. TDP-43 binds to thousands of pre-messenger RNA/mRNA targets, with high affinity for GU-rich sequences, including autoregulation of its own mRNA via binding to 3′ untranslated region. Reduction in TDP-43 from an otherwise normal adult nervous system alters the splicing or expression levels of more than 1,500 RNAs, including long intron-containing transcripts. See Melamed et al., Nat Neurosci. (2019), 22(2):180-190.

In affected neurons in most instances of ALS and approximately 45% of patients with FTD, cytoplasmic accumulation and nuclear loss of TDP-43 have been reported. See Melamed et al., Nat Neurosci. (2019), 22(2):180-190. Moreover, TDP-43 has been shown to regulate expression of the neuronal growth-associated factor stathmin-2. See Melamed (2019); see also Klim et al., Nat Neurosci. (2019), 22(2):167-179. TDP-43 disruption is shown to drive premature polyadenylation and aberrant splicing in intron 1 of stathmin-2 pre-mRNA, producing truncated mRNA and loss of functional STMN2 protein. See Melamed (2019). STMN2 encodes a protein necessary for normal motor neuron outgrowth and repair. See Melamed (2019); see also Klim (2019).

The stathmin-2 gene is annotated to contain five constitutive exons (Refseq ID: NM 001199214.1) plus a proposed alternative exon between exons 4 and 5. See Melamed (2019); see also Klim (2019). Reduction or mutation in TDP-43 induces a new spliced exon, mapping within intron 1. See Melamed (2019); see also Klim (2019). This new exon (denoted as “exon 2a” or “cryptic exon”) appears in STMN2 pre-mRNA when TDP-43 is depleted or endogenous TDP-43 has a N352 mutation. See Melamed (2019); see also Klim (2019). The cryptic exon in STMN2 pre-mRNA contains a cryptic polyadenylation sequence, which results in premature polyadenylation of the pre-mRNA. See Melamed (2019); see also Klim (2019). This prematurely polyadenylated RNA includes 227 nucleotides originating from the cryptic exon with its predicted 16 amino acid translation product initiating at the normal AUG codon in exon 1 and ending 11 codons into the cryptic exon. See Melamed (2019); see also Klim (2019).

Present invention provides inhibitors of STMN2 transcripts that include a cryptic exon, for treatment of neurological diseases or disorders.

### SUMMARY

Described herein are oligonucleotide inhibitors. In various embodiments, the oligonucleotide targets a transcript for the treatment of neurological diseases, including motor neuron diseases, and/or neuropathies. For example, inhibitors of the transcript can be used to treat PD, ALS, FTD, and ALS with FTD. In various embodiments, the oligonucleotide inhibitors are antisense oligonucleotides. In various embodiments, the oligonucleotide inhibitors target a Stathmin-2 (STMN2) transcript. In some embodiments, the STMN2 transcript includes a cryptic exon, such as the cryptic exon with a sequence identified below in SEQ ID NO: 447.

Additionally disclosed herein is a compound comprising an oligonucleotide comprising linked nucleosides with at least a 19 contiguous nucleobase sequence that is at least 90% (e.g., 90%, 91%, 92%, 93%, 94%, 95%, 96%, 97%, 98%, 99% or 100%) complementary to an equal length portion of a transcript with at least 90% (e.g., 90%, 91%, 92%, 93%, 94%, 95%, 96%, 97%, 98%, 99% or 100%) identity to SEQ ID NO: 944, or to a contiguous 19 to 50 nucleobase portion of SEQ ID NO: 944, wherein at least one nucleoside linkage of the linked nucleosides is a non-natural linkage. Additionally disclosed herein is an oligonucleotide comprising linked nucleosides with at least a 19 contiguous nucleobase sequence that is at least 90% (e.g., 90%, 91%, 92%, 93%, 94%, 95%, 96%, 97%, 98%, 99% or 100%) complementary to an equal length portion of a transcript with at least 90% (e.g., 90%, 91%, 92%, 93%, 94%, 95%, 96%, 97%, 98%, 99% or 100%) identity to SEQ ID NO: 944, or to a contiguous 19 to 50 nucleobase portion of SEQ ID NO: 944, wherein at least one nucleoside linkage of the linked nucleosides is a non-natural linkage.

In various embodiments, the nucleobase sequence comprises a portion of at least 10 contiguous nucleobases that shares at least 90% (e.g., 90%, 91%, 92%, 93%, 94%, 95%, 96%, 97%, 98%, 99% or 100%) identity with an equal length portion of any one of SEQ ID NOs: 1-446, SEQ ID NOs: 894-918, SEQ ID NOs: 945-1390, or SEQ ID NOs: 1392-1432. In various embodiments, the nucleobase sequence comprises a portion of at least 11, 12, 13, 14, 15, 16, 17, 18, 19, 20, 21, 22, 23, 24, or 25 contiguous nucleobases that shares at least 90% (e.g., 90%, 91%, 92%, 93%, 94%, 95%, 96%, 97%, 98%, 99% or 100%) identity with an equal length portion of any one of SEQ ID NOs: 1-446, SEQ ID NOs: 894-918, SEQ ID NOs: 945-1390, or SEQ ID NOs: 1392-1432.

In various embodiments, the nucleobase sequence comprises a portion of at least 10 contiguous nucleobases that shares at least 90% (e.g., 90%, 91%, 92%, 93%, 94%, 95%, 96%, 97%, 98%, 99% or 100%) identity with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344, wherein at least one nucleoside linkage of the linked nucleosides is a non-natural linkage. In various embodiments, the nucleobase sequence comprises a portion of at least 11, 12, 13, 14, 15, 16, 17, 18, 19, 20, 21, 22, 23, 24, or 25 contiguous nucleobases that shares at least 90% (e.g., 90%, 91%, 92%, 93%, 94%, 95%, 96%, 97%, 98%, 99% or 100%) identity with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344.

Additionally disclosed herein is a compound comprising an oligonucleotide comprising linked nucleosides with at least a 19 contiguous nucleobase sequence, wherein the nucleobase sequence comprises a portion of at least 10 contiguous nucleobases that shares at least 90% (e.g., 90%, 91%, 92%, 93%, 94%, 95%, 96%, 97%, 98%, 99% or 100%) identity to an equal length portion of any one of SEQ ID NOs: 894-918 or SEQ ID NOs: 1392-1432. Additionally disclosed herein is an oligonucleotide comprising linked nucleosides with at least a 19 contiguous nucleobase sequence, wherein the nucleobase sequence comprises a portion of at least 10 contiguous nucleobases that shares at least 90% (e.g., 90%, 91%, 92%, 93%, 94%, 95%, 96%, 97%, 98%, 99% or 100%) identity to an equal length portion of any one of SEQ ID NOs: 894-918 or SEQ ID NOs: 1392-1432. In various embodiments, the nucleobase sequence comprises a portion of at least 11, 12, 13, 14, 15, 16, 17, 18, 19, 20, 21, 22, 23, 24, or 25 contiguous nucleobases that shares at least 90% (e.g., 90%, 91%, 92%, 93%, 94%, 95%, 96%, 97%, 98%, 99% or 100%) identity to of any one of SEQ ID NOs: 894-918 or SEQ ID NOs: 1392-1432.

Additionally disclosed herein is a compound comprising an oligonucleotide comprising linked nucleosides with at least a 19 contiguous nucleobase sequence, wherein the nucleobase sequence comprises a portion of at least 10 contiguous nucleobases that is at least 90% (e.g., 90%, 91%, 92%, 93%, 94%, 95%, 96%, 97%, 98%, 99% or 100%) complementary to an equal length portion of nucleobases within any one of positions 121-144, 144-168, 146-170, 150-170, 150-172, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. Additionally disclosed herein is an oligonucleotide comprising linked nucleosides with a nucleobase sequence with at least a 19 contiguous nucleobase sequence, wherein the nucleobase sequence comprises a portion of at least 10 contiguous nucleobases that is at least 90% (e.g., 90%, 91%, 92%, 93%, 94%, 95%, 96%, 97%, 98%, 99% or 100%) complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944.

In various embodiments, the portion of the nucleobase sequence is 100% complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. In various embodiments, the portion of the nucleobase sequence is 100% complementary to an equal length portion of nucleobases within any one of positions 144-164, 144-166, 145-167, 146-166, 146-168, 147-165, or 148-168 of SEQ ID NO: 944. In various embodiments, the portion of the nucleobase sequence is 100% complementary to an equal length portion of nucleobases within any one of positions 173-191, 173-193, 173-195, 173-197, 175-195, 175-197, 177-197, or 179-197 of SEQ ID NO: 944.

In various embodiments, the portion of the nucleobase sequence is 100% complementary to an equal length portion of nucleobases within any one of positions 185-205, 187-209, 189-209, 185-207, 197-217, 197-219, or 191-209 of SEQ ID NO: 944. In various embodiments, the portion of the nucleobase sequence is 100% complementary to an equal length portion of nucleobases within any one of positions 237-255, 237-257, 237-259, 239-259, 239-261, 241-261, 237-257, 249-269, 249-271, 252-272, 252-274, or 243-261 of SEQ ID NO: 944. In various embodiments, the nucleobase sequence comprises a portion of at least 11, 12, 13, 14, 15, 16, 17, 18, 19, 20, 21, 22, 23, 24, or 25 contiguous nucleobases that is complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. In various embodiments, the nucleobase sequence comprises a portion of at least 11, 12, 13, 14, 15, 16, 17, 18, 19, 20, 21, 22, 23, 24, or 25 contiguous nucleobases that is complementary to an equal length portion of nucleobases within any one of positions 144-164, 144-166, 145-167, 146-166, 146-168, 147-165, 148-168, 173-191, 173-193, 173-195, 173-197, 175-195, 175-197, 177-197, 179-197, 185-205, 185-207, 197-217, 197-219, 187-209, 189-209, 191-209, 237-255, 237-257, 237-259, 239-259, 239-261, 241-261, 237-257, 249-269, 249-271, 252-272, 252-274, or 243-261 of SEQ ID NO: 944.

In various embodiments, the oligonucleotide is 19 and 40 nucleosides in length. In various embodiments, the oligonucleotide comprises at least one nucleoside linkage selected from the group consisting of a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, or any combination(s) thereof. In various embodiments, at least two, three, or four internucleoside linkages of the oligonucleotide are phosphodiester internucleoside linkages. In various embodiments, the oligonucleotide comprises at least two, three, or four modified internucleoside linkages.

In various embodiments, each of the modified internucleoside linkage of the oligonucleotide is independently selected from a phosphorothioate linkage, a phosphoramidate linkage, a phosphoramidothiate linkage, a phosphorodiamidate. In various embodiments, all internucleoside linkages of the oligonucleotide are phosphorothioate linkages. In various embodiments, the phosphorothioate internucleoside linkage is in one of a Rp configuration or a Sp configuration. In various embodiments, the oligonucleotide comprises at least one modified nucleobase. In various embodiments, the at least one modified nucleobase is 5-methyl cytosine, pseudouridine, or 5-methoxyuridine.

In various embodiments, the oligonucleotide comprises at least one modified sugar moiety. In various embodiments, the modified sugar moiety is one of a 2′-OMe (2′-OCH₃ or 2′-O-methyl) modified sugar moiety, bicyclic sugar moiety, 2′-O-(2-methoxyethyl) (2′-O(CH₂)₂OCH₃ (2′MOE)), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

In various embodiments, wherein the oligonucleotide comprises three linked nucleosides that are linked through phosphodiester internucleoside linkages at the 5′ end and three linked nucleosides that are linked through phosphodiester internucleoside linkages at the 3′ end. In various embodiments, the oligonucleotide comprises one or more 2′-O-(2-methoxyethyl) nucleosides that are linked through phosphorothioate internucleoside linkages. In some embodiments, all cytosine nucleosides in a STMN2 antisense oligonucleotide of the present invention comprise modified sugar moiety comprising 2′-MOE, all nucleosides comprise modified nucleobase 5-methyl cytosine, and all internucleoside linkages are phosphorothioate linkage. In various embodiments, the oligonucleotide comprises three linked nucleosides that are linked through phosphorothioate internucleoside linkages at the 5′ end and three linked nucleosides that are linked through phosphorothioate internucleoside linkages at the 3′ end. In various embodiments, the oligonucleotide comprises five linked nucleosides that are linked through phosphodiester internucleoside linkages. In various embodiments, the each of the five linked nucleosides are 2′-O-(2-methoxyethyl) (2′MOE) nucleosides. In various embodiments, each of the linked nucleosides of the oligonucleotide are 2′-O-(2-methoxyethyl) (2′MOE) nucleosides.

In various embodiments, the oligonucleotide exhibits at least a 30%, 40%, 50%, 60%, 70%, 80%, or 90% increase of full length STMN2 transcript or STMN2 protein. In various embodiments, the oligonucleotide exhibits at least a 100% increase of full length STMN2 transcript or STMN2 protein. In various embodiments, the oligonucleotide exhibits at least a 200% increase of full length STMN2 transcript or STMN2 protein. In various embodiments, the oligonucleotide exhibits at least a 300% increase of full length STMN2 transcript or STMN2 protein. In various embodiments, the oligonucleotide exhibits at least a 400% increase of full length STMN2 transcript or STMN2 protein. In various embodiments, increase of the full length STMN2 protein is measured in comparison to a reduced level of full length STMN2 protein achieved using a TDP43 antisense oligonucleotide. In various embodiments, the oligonucleotide exhibits at least a 10%, 20%, 30%, 40%, 50%, 60%, 70%, 80%, 90%, or 100% rescue of full length STMN2 transcript or STMN2 protein. In various embodiments, the oligonucleotide exhibits at least a 50%, 60%, 70%, 80%, or 90% reduction of the STMN2 transcript with the cryptic exon.

Additionally disclosed herein is a pharmaceutical composition comprising one or more of the oligonucleotides described above, or a pharmaceutically acceptable salt thereof, and a pharmaceutically acceptable excipient. Additionally disclosed herein is a method of treating a neurological disease and/or a neuropathy in a patient in need thereof, the method comprising administering to the patient an oligonucleotide of any of the oligonucleotides described above or a pharmaceutically acceptable salt thereof, or a pharmaceutical composition described above.

In various embodiments, the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD). In various embodiments, the neuropathy is chemotherapy induced neuropathy.

Additionally disclosed herein is a method of restoring axonal outgrowth and/or regeneration of a neuron, the method comprising exposing the motor neuron to an oligonucleotide of any of the oligonucleotides described above or a pharmaceutically acceptable salt thereof, or a pharmaceutical composition described above. Additionally disclosed herein is a method of increasing, promoting, stabilizing, or maintaining STMN2 expression and/or function in a neuron, the method comprising exposing the cell to an oligonucleotide of any of the oligonucleotides described above or a pharmaceutically acceptable salt thereof, or a pharmaceutical composition described above. In various embodiments, the neuron is a neuron of a patient in need of treatment of a neurological disease and/or a neuropathy. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD) . In various embodiments, the neuropathy is chemotherapy induced neuropathy.

In various embodiments, the exposing is performed in vivo or ex vivo. In various embodiments, the exposing comprises administering a STMN2 oligonucleotide (STMN2 AON) disclosed herein or a pharmaceutical composition thereof to a patient in need thereof. In various embodiments, a STMN2 oligonucleotide or a pharmaceutical composition thereof is administered topically, parenterally (e.g., subcutaneous, intramuscular, intradermal, intraduodenal, or intravenous), intralesionally, intrathecally, intracisternally, orally, rectally, buccally, sublingually, vaginally, pulmonarily, intratracheally, intranasally, transdermally, or intraduodenally. In various embodiments, a STMN2 oligonucleotide or a pharmaceutical composition thereof is administered orally. In various embodiments, a therapeutically effective amount of a STMN2 oligonucleotide or a pharmaceutical composition thereof is administered intrathecally or intracisternally.

In various embodiments, the patient is a human. In various embodiments, the pharmaceutical composition is suitable for topical, intrathecal, intracisternal, parenteral (e.g., subcutaneous, intramuscular, intradermal, intraduodenal, or intravenous), intralesional, oral, pulmonary, intratracheal, intranasal, transdermal, rectal, buccal, sublingual, vaginal, or intraduodenal administration.

Additionally disclosed herein is a use of a STMN2 oligonucleotide or a pharmaceutically acceptable salt thereof, or a pharmaceutical composition described above in the manufacture of a medicament for the treatment of neurological disease or a neuropathy. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD). In various embodiments, the neuropathy is chemotherapy induced neuropathy.

Additionally disclosed herein is a method of treating a neurological disease or a neuropathy in a patient in need thereof, the method comprising administering to a patient in need thereof a therapeutically effective amount of a STMN2 oligonucleotide or a pharmaceutically acceptable salt thereof, or a pharmaceutical composition described above. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD). In various embodiments, the neuropathy is chemotherapy induced neuropathy. In various embodiments, the pharmaceutical composition is administered topically, parenterally (e.g., subcutaneous, intramuscular, intradermal, intraduodenal, or intravenous), intralesionally, orally, pulmonarily, rectally, buccally, sublingually, vaginally, intratracheally, intranasally, intracisternally, intrathecally, transdermally, or intraduodenally. In various embodiments, the pharmaceutical composition is administered intrathecally or intracisternally. In various embodiments, a therapeutically effective amount of a STMN2 oligonucleotide or a pharmaceutical composition thereof is administered intrathecally or intracisternally. In various embodiments, the patient is human.

Additionally disclosed herein is a STMN2 oligonucleotide or a pharmaceutically acceptable salt thereof, for use as a medicament in the treatment of a neurological disease or a neuropathy. In certain embodiments, the present disclosure provides a STMN2 oligonucleotide or a pharmaceutically acceptable salt thereof, for use in the treatment of a neurological disease or a neuropathy. In various embodiments, the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD). In various embodiments, the neuropathy is chemotherapy induced neuropathy.

Additionally disclosed herein is a STMN2 oligonucleotide comprising linked nucleosides with a nucleobase sequence of any one of SEQ ID NOs: 1-446, SEQ ID NOs: 894-918, SEQ ID NOs: 945-1390, or SEQ ID NOs: 1392-1432, or a pharmaceutically acceptable salt thereof; wherein the oligonucleotide comprises at least one nucleoside linkage selected from the group consisting of: a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, and/or wherein at least one nucleoside of the linked nucleosides is substituted with a component selected from the group consisting of a 2′-O-(2-methoxyethyl) nucleoside (2′-O-methoxyethylribonucleosides (2′-MOE)), a 2′-O-methyl nucleoside, a 2′-deoxy-2′-fluoro nucleoside, a 2′-fluoro-β-D-arabinonucleoside, a locked nucleic acid (LNA), constrained methoxyethyl (cMOE), constrained ethyl (cET), and a peptide nucleic acid (PNA).

In various embodiments, at least one internucleoside linkage of the oligonucleotide is a phosphorothioate linkage. In various embodiments, the oligonucleotide comprises three linked nucleosides that are linked through phosphodiester internucleoside linkages at the 5′ end and three linked nucleosides that are linked through phosphodiester internucleoside linkages at the 3′ end. In various embodiments, the oligonucleotide comprises one or more 2′-O-(2-methoxyethyl) nucleosides that are linked through phosphorothioate internucleoside linkages. In various embodiments, the oligonucleotide comprises five linked nucleosides that are linked through phosphodiester internucleoside linkages. In various embodiments, each of the five linked nucleosides are 2′-O-(2-methoxyethyl) (2′MOE) nucleosides. In various embodiments, each of the linked nucleosides of the oligonucleotide are 2′-O-(2-methoxyethyl) (2′MOE) nucleosides. In various embodiments, all internucleoside linkages of the oligonucleotide are phosphorothioate linkages, optionally wherein each of the linked nucleosides of the oligonucleotide are 2′-O-(2-methoxyethyl) (2′ -MOE) nucleosides.

Additionally disclosed herein is a pharmaceutical composition comprising the oligonucleotide of any oligonucleotide described above, or a pharmaceutically acceptable salt thereof, and a pharmaceutically acceptable excipient. Additionally disclosed herein is STMN2 oligonucleotide or a pharmaceutically acceptable salt thereof capable of increasing, restoring, or stabilizing expression of the STMN2 mRNA capable of translation of a functional STMN2 and/or activity and/or function of STMN2 protein in a cell or a human patient of a neurological disease or disorder, wherein the level of increase, restoration, or stabilization of expression and/or activity and/or function is sufficient for use of the oligonucleotide as a medicament for the treatment of neurological disease or disorder. In various embodiments, wherein the oligonucleotide comprises one or more chiral centers and/or double bonds. In various embodiments, the oligonucleotide exist as stereoisomers selected from geometric isomers, enantiomers, and diastereomers.

Additionally disclosed herein is a method of treating a neurological disease and/or a neuropathy in a patient in need thereof, the method comprising administering to a patient in need thereof a therapeutically effective amount of a STMN2 oligonucleotide or a pharmaceutically acceptable salt thereof, or a pharmaceutical composition described above, in combination with a second therapeutic agent selected from Riluzole (Rilutek), Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (EL RIO). ZILUCOPLAN (RA101495), dual AON intrathecal administration (e.g., BIB067, BIB078), BLIB100, levodopa/carbidopa, dopaminergic agents (e.g., ropinirole, pramipexole, rotigotine), medroxyprogesterone, KCNQ2/KCNQ3 openers, anticonvulsants and psychostimulant agents, and/or a therapy (e.g., selected from breathing care, physical therapy, occupational therapy, speech therapy, nutritional support), for treating said neurologic disease.

In various embodiments, the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD). In various embodiments, the neuropathy is chemotherapy induced neuropathy.

### BRIEF DESCRIPTION OF THE DRAWINGS

FIG. 1A is a schematic depiction of portions of the STMN2 transcript and STMN2 antisense oligonucleotides that are designed to target certain portions of the STMN2 transcript. FIG. 1B is another schematic depiction of portions of the STMN2 transcript and STMN2 antisense oligonucleotides that are designed to target certain portions of the STMN2 transcript in SY5Y cells. FIG. 1C is yet another schematic depiction of portions of the STMN2 transcript and STMN2 antisense oligonucleotides that are designed to target certain portions of the STMN2 transcript in human motor neurons. In each of FIG. 1A, 1B, and 1C the solid line represents tested STMN2 AON that increased STMN2-FL mRNA expression by greater than 50% over TDP43 AON treated alone. The dotted line represents tested STMN2 AON that increased STMN2-FL (full length) mRNA less than 50% over TDP43 AON treated alone.

FIG. 2 is a bar graph showing the results of RT-qPCR analysis of TDP43 and STMN2 full-length mRNA levels in the presence of TDP43 antisense, and restoration of the full-length STMN2 transcript in the presence of 6 different STMN2 antisense oligonucleotides (QSN-36, QSN-55, QSN-177, QSN-203, QSN-244, and QSN-395).

FIG. 3 is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 6 different STMN2 antisense oligonucleotides (QSN-173, QSN-181, QSN-197, QSN-215, QSN-385, and QSN-400).

FIG. 4 is a bar graph showing the results of RT-qPCR analysis of STMN2 full-length mRNA levels in the presence of TDP43 antisense, and restoration of the full-length STMN2 transcript in the presence of 6 different STMN2 antisense oligonucleotides (QSN-173, QSN-181, QSN-197, QSN-215, QSN-385, and QSN-400).

FIG. 5A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 6 different STMN2 antisense oligonucleotides (QSN-185, QSN-209, QSN-237, QSN-252, QSN-380, and QSN-390).

FIG. 5B is a bar graph showing the results of RT-qPCR analysis of STMN2 full-length mRNA levels in the presence of TDP43 antisense, and restoration of the full-length STMN2 transcript in the presence of 6 different STMN2 antisense oligonucleotides (QSN-185, QSN-209, QSN-237, QSN-252, QSN-380, and QSN-390).

FIG. 6A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 2 different STMN2 antisense oligonucleotides (QSN-144 and QSN-237) over two duplicate experiments.

FIG. 6B is a bar graph showing the results of RT-qPCR analysis of STMN2 full-length mRNA levels in the presence of TDP43 antisense, and restoration of the full-length STMN2 transcript in the presence of 2 different STMN2 antisense oligonucleotides (QSN-144 and QSN-237) over two duplicate experiments.

FIG. 7A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 5 different STMN2 antisense oligonucleotides (QSN-36, QSN-173, QSN-177, QSN-181, and QSN-185).

FIG. 7B is a bar graph showing the results of RT-qPCR analysis of STMN2 full-length mRNA levels in the presence of TDP43 antisense, and restoration of the full-length STMN2 transcript in the presence of 5 different STMN2 antisense oligonucleotides (QSN-36, QSN-173, QSN-177, QSN-181, and QSN-185).

FIG. 8A is a bar graph showing the results of RT-qPCR analysis of STMN2 transcript with cryptic exon mRNA levels in the presence of TDP43 antisense, and reduction of the STMN2 transcript with cryptic exon mRNA levels in the presence of 5 different STMN2 antisense oligonucleotides (QSN-197, QSN-203, QSN-237, QSN-380, and QSN-395).

FIG. 8B is a bar graph showing the results of RT-qPCR analysis of STMN2 full-length mRNA levels in the presence of TDP43 antisense, and restoration of the full-length STMN2 transcript in the presence of 5 different STMN2 antisense oligonucleotides (QSN-197, QSN-203, QSN-237, QSN-380, and QSN-395).

FIG. 9A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels in the presence of 3 different STMN2 antisense oligonucleotides (QSN-144, QSN-173, and QSN-237).

FIG. 9B is a bar graph showing the results of RT-qPCR analysis of TDP43 and STMN2 full-length mRNA levels in the presence of TDP43 siRNA and TDP43 antisense, and restoration of the full-length STMN2 transcript in the presence of 3 different STMN2 antisense oligonucleotides (QSN-144, QSN-173, and QSN-237).

FIG. 10A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a QSN-181 STMN2 antisense oligonucleotide.

FIG. 10B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a QSN-181 STMN2 antisense oligonucleotide.

FIG. 11A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a QSN-185 STMN2 antisense oligonucleotide.

FIG. 11B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a QSN-185 STMN2 antisense oligonucleotide.

FIG. 12A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a QSN-197 STMN2 antisense oligonucleotide.

FIG. 12B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a QSN-197 STMN2 antisense oligonucleotide.

FIG. 13A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a QSN-144 STMN2 antisense oligonucleotide.

FIG. 13B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a QSN-144 STMN2 antisense oligonucleotide.

FIG. 14A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a QSN-173 STMN2 antisense oligonucleotide.

FIG. 14B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a QSN-173 STMN2 antisense oligonucleotide.

FIG. 15A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels across different dosages of a QSN-237 STMN2 antisense oligonucleotide.

FIG. 15B is a bar graph showing the results of RT-qPCR analysis of TDP43 and STMN2 full-length mRNA levels in the presence of TDP43 siRNA and TDP43 antisense, and restoration of the full-length STMN2 transcript across different dosages of a QSN-237 STMN2 antisense oligonucleotide.

FIG. 16 is a protein blot and quantified bar graph showing the normalized quantity of STMN2 full-length mRNA levels in the presence of TDP43 siRNA and TDP43 antisense, and restoration of the full-length STMN2 transcript for 2 different STMN2 antisense oligonucleotides (QSN-173 and QSN237).

FIG. 17A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a QSN-237 STMN2 antisense oligonucleotide.

FIG. 17B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a QSN-237 STMN2 antisense oligonucleotide.

FIG. 18A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a QSN-185 STMN2 antisense oligonucleotide.

FIG. 18B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a QSN-185 STMN2 antisense oligonucleotide.

FIG. 19A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a QSN-173 STMN2 antisense oligonucleotide.

FIG. 19B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a QSN-173 STMN2 antisense oligonucleotide.

FIG. 20A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a QSN-237 STMN2 antisense oligonucleotide.

FIG. 20B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a QSN-237 STMN2 antisense oligonucleotide.

FIG. 21A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a QSN-173 STMN2 antisense oligonucleotide.

FIG. 21B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a QSN-173 STMN2 antisense oligonucleotide.

FIG. 22A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different variants of a QSN-144 STMN2 antisense oligonucleotide.

FIG. 22B is a bar graph showing the results of RT-qPCR analysis of TDP43 and STMN2 full-length mRNA levels in the presence of TDP43 siRNA and TDP43 antisense, and restoration of the full-length STMN2 transcript using different variants of a QSN-144 STMN2 antisense oligonucleotide.

FIG. 23 shows the dose response curve illustrating increasing restoration of full length STMN2 transcript with increasing concentrations of STMN2 AON.

FIG. 24A shows a Western blot assay demonstrating the qualitative increase of full length STMN2 protein in response to higher concentrations of STMN2 AON.

FIG. 24B shows the quantitated levels of full length STMN2 protein normalized to GAPDH in response to different concentrations of STMN2 AON.

FIG. 25A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-144 STMN2 AONs and AON variants.

FIG. 25B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-144 STMN2 AONs and AON variants.

FIG. 26A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-173 STMN2 AONs and AON variants.

FIG. 26B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-173 STMN2 AONs and AON variants.

FIG. 27A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-185 STMN2 AONs and AON variants.

FIG. 27B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-185 STMN2 AONs and AON variants.

FIG. 28A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-237 STMN2 AONs and AON variants.

FIG. 28B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-237 STMN2 AONs and AON variants.

FIG. 29A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different STMN2 AONs (QSN-31, QSN-41, and QSN-46).

FIG. 29B is a bar graph showing the results of RT-qPCR analysis of TDP43 and STMN2 full-length mRNA levels in the presence of TDP43 siRNA and TDP43 antisense, and restoration of the full-length STMN2 transcript using different STMN2 AONs (QSN-31, QSN-41, and QSN-46).

FIG. 30 is a bar graph showing reversal of cryptic exon induction in human motor neurons using QSN-237 STMN2 antisense oligonucleotide even in view of increasing proteasome inhibition.

FIGS. 31A and 31B show bar graphs showing the results of RT-qPCR analysis of STMN2 transcript with cryptic exon mRNA levels and STMN2 full-length mRNA levels, which demonstrate reduction of the STMN2 transcript with cryptic exon mRNA levels and restoration of the full-length STMN2 transcript using different STMN2 AONs and AON variants.

FIG. 32 is a bar graph showing the results of a western blot analysis of STMN2 protein levels, which demonstrates restoration of the full-length STMN2 protein using different STMN2 AONs and AON variants.

FIG. 33A is a bar graph showing the results of RT-qPCR analysis of STMN2 transcript with cryptic exon mRNA expression in human motor neurons, which demonstrates reduction of the STMN2 transcript with cryptic exon mRNA levels using different STMN2 AONs (QSN-31, QSN-41, and QSN-46).

FIG. 33B is a bar graph showing the results of RT-qPCR analysis of STMN2 full-length mRNA levels, which demonstrates restoration of the full-length STMN2 transcript using different STMN2 AONs (QSN-31, QSN-41, and QSN-46).

FIG. 34A is a bar graph showing the results of RT-qPCR analysis of STMN2 transcript with cryptic exon mRNA expression in human motor neurons, which demonstrates reduction of the STMN2 transcript with cryptic exon mRNA levels using different STMN2 AONs (QSN-146, QSN-150, and QSN-169).

FIG. 34B is a bar graph showing the results of RT-qPCR analysis of STMN2 full-length mRNA levels, which demonstrates the restoration of the full-length STMN2 transcript using different STMN2 AONs (QSN-146, QSN-150, and QSN-169).

FIG. 34C is a bar graph showing the results of RT-qPCR analysis of STMN2 transcript with cryptic exon mRNA expression in human motor neurons, which demonstrates reduction of the STMN2 transcript with cryptic exon mRNA levels using different STMN2 AONs (QSN-170, QSN-171, and QSN-172).

FIG. 34D is a bar graph showing the results of RT-qPCR analysis of STMN2 full-length mRNA levels, which demonstrates the restoration of the full-length STMN2 transcript using different STMN2 AONs (QSN-170, QSN-171, and QSN-172).

FIG. 34E is a bar graph showing the results of RT-qPCR analysis of STMN2 transcript with cryptic exon mRNA expression in human motor neurons, which demonstrates reduction of the STMN2 transcript with cryptic exon mRNA levels using different STMN2 AONs (QSN-249).

FIG. 34F is a bar graph showing the results of RT-qPCR analysis of STMN2 full-length mRNA levels, which demonstrates the restoration of the full-length STMN2 transcript using different STMN2 AONs (QSN-249).

### DETAILED DESCRIPTION

The features and other details of the disclosure will now be more particularly described. Certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

The terms “treat,” “treatment,” “treating,” and the like are used herein to generally mean obtaining a desired pharmacological and/or physiological effect. The effect may be therapeutic in terms of partially or completely curing a disease and/or adverse effect attributed to the disease. The term “treatment” as used herein covers any treatment of a disease in a mammal, particularly a human, and includes: (a) inhibiting the disease, i.e., preventing the disease from increasing in severity or scope; (b) relieving the disease, i.e., causing partial or complete amelioration of the disease; or (c) preventing relapse of the disease, i.e., preventing the disease from returning to an active state following previous successful treatment of symptoms of the disease or treatment of the disease.

“Preventing” includes delaying the onset of clinical symptoms, complications, or biochemical indicia of the state, disorder, disease, or condition developing in a subject that may be afflicted with or predisposed to the state, disorder, disease, or condition but does not yet experience or display clinical or subclinical symptoms of the state, disorder, disease, or condition. “Preventing” includes prophylactically treating a state, disorder, disease, or condition in or developing in a subject, including prophylactically treating clinical symptoms, complications, or biochemical indicia of the state, disorder, disease, or condition in or developing in a subject.

The term “pharmaceutically acceptable carrier” or “pharmaceutically acceptable excipient” as used herein interchangeably refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one biologically active compound, for example, a STMN2 antisense oligonucleotide (AON), as disclosed herein formulated together with one or more pharmaceutically acceptable excipients.

“Individual,” “patient,” or “subject” are used interchangeably and include any animal, including mammals, preferably mice, rats, other rodents, rabbits, dogs, cats, swine, cattle, sheep, horses, or non-human primates, and most preferably humans. The compounds of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, non-human primates, and the like). In some embodiments, the mammal treated in the methods of the invention is desirably a mammal in whom modulation of STMN2 expression and/or activity is desired.

The term “STMN2 oligonucleotide,” “STMN2 antisense oligonucleotide,” or “STMN2 AON” refers to an oligonucleotide that is capable of increasing, restoring, or stabilizing full-length STMN2 activity e.g., full length STMN2 expression, for example, full length STMN2 mRNA and/or full length STMN2 protein expression. Generally, a STMN2 oligonucleotide reduces the level of mature STMN2 transcripts with a cryptic exon by targeting a STMN2 transcript comprising a cryptic exon. A patient suffering from ALS, FTD, ALS with FTD, or another neurological or motor neuron disease can be a patient that is diagnosed with the disease or that displays symptoms of the disease. A patient suffering from ALS, FTD, ALS with FTD, or another neurological or motor neuron disease can be a patient that previously suffered from the disease and, after recovering or experiencing complete or partial amelioration of the disease and/or disease symptoms, experiences a complete or partial relapse of the disease or disease symptoms. A patient suffering from ALS, FTD, ALS with FTD, or another neurological or motor neuron disease or condition can be a patient that harbors a genetic mutation associated with manifestation of the disease or condition. For example, a patient suffering from ALS can be a patient that harbors a genetic mutation in any of SOD1, C9orf72, Ataxin 2 (ATXN2), Charged Multivesicular Body Protein 2B (CHMP2B), Dynactin 1 (DCTN1), Human Epidermal Growth Factor Receptor 4 (ERBB4), FIG4 phosphoinositide 5-phosphatase (FIG4), NIMA related kinase 1 (NEK1), Heterogeneous nuclear ribonucleoprotein Al (HNRNPA1), Neurofilament Heavy (NEFH), Peripherin (PRPH), TAR DNA binding protein 43 (TDP43 or TARDBP), Fused in Sarcoma (FUS), Ubiquilin-2 (UBQLN2), Kinesin Family Member 5A (KIFSA), Valosin-Containing Protein (VCP), Alsin (ALS2), Senataxin (SETX), Sigma Non-Opioid Intracellular Receptor 1 (SIGMAR1), Survival of Motor Neuron 1, Telomeric (SMN1), Spastic Paraplegia 11, Autosomal Recessive (SPG11), Transient Receptor Potential Cation Channel Subfamily M Member 7 (TRPM7), Vesicle-Associated Membrane Protein-Associated Protein B/C (VAPB), Angiogenin (ANG), Profilin-1 (PFN1), Matrin-3 (MATR3), Coiled-coil-helix-coiled-coil-helix domain Containing 10 (CHCHD10), Tubulin, Alpha 4A (TUBA4A), TBK1, C21orf2, Sequestosome-1 (SQSTM1, also known as Ubiquitin-binding protein p62), and/or optineurin (OPTN), in particular, where the mutation is associated with ALS or a high risk of developing ALS.

A patient at risk of ALS, FTD, ALS with FTD, or another neurological or motor neuron disease can include those patients with a familial history of the disease or a genetic predisposition to the disease (e.g., a patient that harbors a genetic mutation associated with high disease risk, for example), or patients exposed to environmental factors that increase disease risk. For example, a patient may be at risk of ALS if the patient harbors a mutation in any of genes encoding SOD1, C9orf72, ATXN2, CHMP2B, DCTN1, ERBB4, FIG4, HNRNPA1, NEFH, PRPH, NEK1, TDP43, FUS, UBQLN2, KIFSA, VCP, ALS2, SETX, SIGMAR1, SMN1, SPG11, TRPM7, VAPB, ANG, PFN1, MATR3, CHCHD10, TUBA4A, TBK1, SQSTM1, C21orf2, and/or OPTN, in particular, where the mutation is associated with ALS or high risk of developing ALS. A patient at risk may also include those patients diagnosed with a disease or condition that has a high comorbidity with ALS, FTD, ALS with FTD, or another neurological or motor neuron disease (for example, a patient suffering from dementia, which is significantly associated with higher odds of a family history of ALS, FTD, and of bulbar onset ALS (see Trojsi, F., et al. (2017) “Comorbidity of dementia with amyotrophic lateral sclerosis (ALS): insights from a large multicenter Italian cohort” J Neural 264: 2224-31)).

As used herein, “STMN2” (also known as Superior Cervical Ganglion-10 Protein, Stathmin-Like 2, SCGN10, SCG10, Neuronal Growth-Associated Protein, Neuron-Specific Growth-Associated Protein, or Protein SCG10 (Superior Cervical Ganglia NEAR Neural Specific 10) refers to the gene or gene products (e.g., protein or mRNA transcript (including pre-mRNA) encoded by the gene) identified by Entrez Gene ID No. 11075 and allelic variants thereof, as well as orthologs found in non-human species (e.g., non-human primates or mice).

In the present specification, the term “therapeutically effective amount” means the amount of the subject inhibitor of STMN2 transcripts that include a cryptic exon that will elicit the biological or medical response of a tissue, system, animal or human that is being sought by the researcher, veterinarian, medical doctor, or other clinician. The inhibitor of STMN2 transcripts that include a cryptic exons of the invention are administered in therapeutically effective amounts to treat and/or prevent a disease, condition, disorder, or state, for example, ALS, FTD, ALS with FTD, or another motor neuron disease or neurological disease or condition. Alternatively, a therapeutically effective amount of an inhibitor of STMN2 transcripts that include a cryptic exon is the quantity required to achieve a desired therapeutic and/or prophylactic effect, such as an amount which results in the prevention of or a decrease in the symptoms associated with a disease associated with reduced STMN2 activity in the motor neurons.

The phrase “oligonucleotide that targets a STMN2 transcript” refers to an oligonucleotide that binds to a STMN2 transcript. In various embodiments, the oligonucleotide binds to a region of a STMN2 transcript. Example regions of a STMN2 transcript are shown in Table 1, which show sequences corresponding to regions of branch points (e.g., branch point 1, 2, and 3) a 3′ splice acceptor region, an ESE binding region, TDP43 binding sites, a cryptic exon, and a Poly A region. In various embodiments, the oligonucleotide binds to a region of a STMN2 transcript with a cryptic exon, the region being located less than 75 nucleobases upstream or downstream to any of the branch points (e.g., branch point 1, 2, and 3) a 3′ splice acceptor region, an ESE binding region, TDP43 binding sites, a cryptic exon, and a Poly A region.

The term “pharmaceutically acceptable salt(s)” as used herein refers to salts of acidic or basic groups that may be present in inhibitors of STMN2 transcripts that include a cryptic exon used in the present compositions. Inhibitors of STMN2 transcripts that include a cryptic exon included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Inhibitors of STMN2 transcripts that include a cryptic exon included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, and lithium salts. Pharmaceutically acceptable salts of the disclosure include, for example, pharmaceutically acceptable salts of STMN2 AONs that include a nucleobase sequence of any of SEQ ID NOs: 1-446, SEQ ID NOs: 894-918, SEQ ID NOs: 945-1390, or SEQ ID NOs: 1392-1432.

Inhibitors of STMN2 transcripts that include a cryptic exon of the disclosure may contain one or more chiral centers, groups, linkages, and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S” (or “Rp” or “Sp”) depending on the configuration of substituents around the stereogenic atom, for example, a stereogenic carbon, phosphorus, or sulfur atom. In some embodiments, one or more linkages of the compound may have a Rp or Sp configuration (e.g., one or more phosphorothioate linkages have either a Rp or Sp configuration). The configuration of each phosphorothioate linkage may be independent of another phosphorothioate linkage (e.g., one phosphorothioate linkage has a Rp configuration and a second phosphorothioate linkage has a Sp configuration). The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. Individual stereoisomers of inhibitors of STMN2 transcripts that include a cryptic exon of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase super critical fluid chromatography, chiral-phase simulated moving bed chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

The inhibitors of STMN2 transcripts that include a cryptic exon disclosed herein can exist in solvated as well as unsolvated forms with pharmaceutically acceptable solvents such as water, ethanol, and the like, and it is intended that the invention embrace both solvated and unsolvated forms.

The disclosure also embraces isotopically labeled compounds of the invention (i.e., isotopically labeled inhibitors of STMN2 transcripts that include a cryptic exon) which are identical to those recited herein, except that one or more atoms are replaced by an atom having an atomic mass or mass number different from the atomic mass or mass number abundantly found in nature. Examples of isotopes that can be incorporated into compounds of the invention include isotopes of hydrogen, carbon, nitrogen, oxygen, phosphorus, fluorine and chlorine, such as ²H, ³H, ¹¹C, ¹³C, ¹⁴C, ¹⁵N, ¹⁸O, ¹⁷O, ³¹P, ³²P, ³³P, ³⁵S, ¹⁸F, and ³⁶Cl, respectively.

Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

As used herein, “2′-O-(2-methoxyethyl)” (also 2′-MOE and 2′-O(CH₂)₂OCH₃ and MOE) refers to an O-methoxyethyl modification of the 2′ position of a furanose ring. A 2′-O-(2-methoxyethyl) is used interchangeably as “2′-O-methoxyethyl” in the present disclosure. A sugar moiety in a nucleoside modified with 2′-MOE is a modified sugar.

As used herein, “2′-MOE nucleoside” (also 2′-O-(2-methoxyethyl) nucleoside) means a nucleoside comprising a 2′-MOE modified sugar moiety.

As used herein, “2′-substituted nucleoside” means a nucleoside comprising a substituent at the 2′-position of the furanose ring other than H or OH. In certain embodiments, 2′ substituted nucleosides include nucleosides with bicyclic sugar modifications.

As used herein, “5-methyl cytosine” (5-MeC) means a cytosine modified with a methyl group attached to the 5 position. A 5-methyl cytosine (5-MeC) is a modified nucleobase.

As used herein, “bicyclic sugar” means a furanose ring modified by the bridging of two atoms. A bicyclic sugar is a modified sugar.

As used herein, “bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

As used herein, “cap structure” or “terminal cap moiety” means chemical modifications, which have been incorporated at either terminus of an antisense compound.

As used herein, “cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

As used herein, “constrained ethyl nucleoside” (also cEt nucleoside) means a nucleoside comprising a bicyclic sugar moiety comprising a 4′-CH(CH₃)—O-2′ bridge.

As used herein, “internucleoside linkage” refers to the covalent linkage between adjacent nucleosides in an oligonucleotide. In some embodiments, as used herein, “non-natural linkage” refers to a “modified internucleoside linkage.”

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “locked nucleic acid” or “LNA” or “LNA nucleosides” means nucleic acid monomers having a bridge (e.g., methylene, ethylene, aminooxy, or oxyimino bridge) connecting two carbon atoms between the 4′ and 2′ position of the nucleoside sugar unit, thereby forming a bicyclic sugar. Examples of such bicyclic sugar include, but are not limited to A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA.

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ₓ— and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R₁)-2′ and 4′-CH₂—N(R₁)—O-2′- bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore, in the case of the bicyclic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

As used herein, “hotspot region” is a range of nucleobases on a target nucleic acid amenable to oligomeric compound-mediated modulation of the splicing of the target nucleic acid.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoosteen or reversed Hoosteen hydrogen bonding between complementary nucleobases.

As used herein, “increasing the amount of activity” refers to more transcriptional expression, more accurate splicing resulting in full length mature mRNA and/or protein expression, and/or more activity relative to the transcriptional expression or activity in an untreated or control sample.

As used herein, “mismatch” or “non-complementary nucleobase” refers to the case when a nucleobase of a first nucleic acid is not capable of pairing with the corresponding nucleobase of a second or target nucleic acid.

As used herein, “linked nucleosides” are nucleosides that are connected through internucleoside linkages in a contiguous sequence (i.e., no additional nucleosides are presented between those that are linked).

As used herein, “modified internucleoside linkage” refers to a substitution or any change from a naturally occurring internucleoside linkage (e.g., a phosphodiester internucleoside bond). “Phosphorothioate linkage” is a modified internucleoside linkage in which one of the non-bridging oxygen atoms of a phosphodiester internucleoside linkage is replaced with a sulfur atom.

As used herein, “modified nucleobase” means any nucleobase other than adenine, cytosine, guanine, thymidine, or uracil. Examples of a modified nucleobase include 5-methyl cytosine, pseudouridine, or 5-methoxyuridine. An “unmodified nucleobase” means the purine bases adenine (A) and guanine (G), and the pyrimidine bases thymine (T), cytosine (C), and uracil (U).

As used herein, a “modified nucleoside” means a nucleoside having, independently, a modified sugar moiety and/or modified nucleobase. A universal base is a modified nucleobase that can pair with any one of the five unmodified nucleobases. Modified nucleosides include abasic nucleosides, which lack a nucleobase.

As used herein, “modified oligonucleotide” means an oligonucleotide comprising at least one modified internucleoside linkage, modified sugar, and/or modified nucleobase.

As used herein, “modified sugar” or “modified sugar moiety” means a modified furanosyl sugar moiety or a modified sugar moiety having other than a furanosyl moiety that can link a nucleobase to another group, such as an internucleoside linkage, conjugate group, or terminal group in an oligonucleotide.

As used herein, “monomer” means a single unit of an oligomer. Monomers include, but are not limited to, nucleosides and nucleotides, whether naturally occurring or modified.

As used herein, “motif” means the pattern of unmodified and modified nucleosides in an antisense compound. p As used herein, “natural sugar moiety” means a sugar moiety found in DNA (2′-H) or RNA (2′-OH).

As used herein, “naturally occurring internucleoside linkage” means a 3′ to 5′ phosphodiester linkage.

As used herein, “non-complementary nucleobase” refers to a pair of nucleobases that do not form hydrogen bonds with one another or otherwise support hybridization.

As used herein, “nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (siRNA), short-hairpin RNA (shRNA), and microRNAs (miRNA).

As used herein, “nucleobase” means a heterocyclic moiety capable of pairing with a base of another nucleic acid.

As used herein, “nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, “nucleobase sequence” means the order of contiguous nucleobases independent of any sugar, linkage, and/or nucleobase modification.

As used herein, “nucleoside” means a nucleobase linked to a sugar. The term “nucleoside” also includes a “modified nucleoside” which has independently, a modified sugar moiety and/or modified nucleobase.

As used herein, “nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non-furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

As used herein, “nucleotide” means a nucleoside having a phosphate group covalently linked to the sugar portion of the nucleoside.

As used herein, “oligomeric compound” or “oligomer” means a polymer of linked monomeric subunits which is capable of hybridizing to at least a region of a nucleic acid molecule.

As used herein, “oligonucleotide” means a polymer of linked nucleosides each of which can be modified or unmodified, independent one from another.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorus-containing and non-phosphorus-containing linkages are well known.

In certain embodiments, antisense compounds targeted to a STMN2 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage. In certain embodiments, the antisense compounds targeted to a STMN2 nucleic acid comprise at least one phosphodiester linkage and at least one phosphorothioate linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S or CF2 with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or 5), 4′-S, 2′-F, 2′-OCH₃, 2′-OCH₂CH₃, 2′-OCH₂ CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂S CH₃, O(CH₂)₂—O—N(Rₘ)(Rₙ), O—CH₂C(═O)N(Rₘ)(Rₙ), and O—CH₂—C(═O)—N(R₁)—(CH₂)₂—N(Rₘ)(Rₙ)—, where each R₁, Rₘ and Rₙ is, independently, H or substituted or unsubstituted C₁-C₁₀ alkyl.

Additional examples of modified sugar moieties include a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt) (4′-CH(CH₃)—O-2′), S-constrained ethyl (S-cEt) 2′-4′-bridged nucleic acid, 4′-CH₂—O—CH₂-2′, 4′ -CH₂—N(R)-2′, 4′-CH(CH₂OCH₃)—O-2′ (“constrained MOE” or “cMOE”), hexitol nucleic acids (HNA), and tricyclic analog (e.g., tcDNA).

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. No. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[—[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl, each Rₐ and R_{b} is, independently, H, a protecting group, hydroxyl, C₁-C₁₂ alkyl, substituted C₁-C₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁).

In certain embodiments, bicyclic nucleosides are further defined by isomeric configuration. For example, a nucleoside comprising a 4′-2′ methylene-oxy bridge, may be in the α-L configuration or in the β-D configuration. Previously, a-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, α-L-methyleneoxy (4′-CH₂—O-2′) BNA, β-D-methyleneoxy (4′-CH₂—O-2′) BNA, ethyleneoxy (4′-(CH₂)₂—O-2) BNA, aminooxy (4′-CH₂—O—N(R)-2′) BNA, oxyamino (4′-CH₂—N(R)—O-2′) BNA, methyl(methyleneoxy) (4′-CH(CH₃)—O-2′) BNA, methylene-thio (4′-CH₂—S-2′) BNA, methylene-amino (4′-CH₂—N(R)-2′) BNA, methyl carbocyclic (4′-CH₂—CH(CH₃)-2′) BNA, and propylene carbocyclic (4′-(CH₂)₃-2′) BNA.

The present disclosure provide, in some embodiments, methods for treating, ameliorating, or preventing a neurological disease such as, but not limited to, ALS, FTD, or ALS with FTD, or treating, ameliorating, or preventing a neurological disease, condition, or a disorder characterized symptoms associated with a neurological disease such as, but not limited to, ALS, FTD, or ALS with FTD, include methods of administering a pharmaceutically acceptable composition, for example, a pharmaceutically acceptable formulation, that includes one or more inhibitors of STMN2 transcripts that include a cryptic exon, to a patient. Inhibitors of STMN2 transcripts that include a cryptic exon can increase, restore, or stabilize STMN2 activity, for example, STMN2 activity, and/or levels of STMN2 expression, for example, STMN2 mRNA and/or protein expression.

The present disclosure also provides pharmaceutical compositions comprising inhibitor of STMN2 transcripts that include a cryptic exon as disclosed herein formulated together with one or more pharmaceutically or cosmetically acceptable excipients. These formulations include those suitable for oral, sublingual, intratracheal, intranasal, transdermal, pulmonary, intrathecal, intracisternal, parenteral (e.g., subcutaneous, intramuscular, intradermal, intraduodenal, or intravenous) or intralesional, administration, transmucosal (e.g., buccal, vaginal, and rectal), or for topical use, e.g., as part of a composition suitable for applying topically to skin and/or mucous membrane, for example, a composition in the form of a gel, a paste, a wax, a cream, a spray, a liquid, a foam, a lotion, an ointment, a topical solution, a transdermal patch, a powder, a vapor, or a tincture. Although the most suitable form of administration in any given case will depend on the degree and severity of the condition being treated and on the nature of the particular inhibitor of STMN2 transcripts that include a cryptic exon being used.

The present disclosure also provides a pharmaceutical composition comprising an inhibitor of STMN2 transcripts that include a cryptic exon, or a pharmaceutically acceptable salt thereof (for example, a STMN2 AON that includes a nucleobase sequence of any of SEQ ID NOs: 1-446, SEQ ID NOs: 894-918, SEQ ID NOs: 945-1390, or SEQ ID NOs: 1392-1432).

The present disclosure also provides methods that include the use of pharmaceutical compositions comprising inhibitor of STMN2 transcripts that include a cryptic exon as disclosed herein (e.g., a STMN2 AON of any one of SEQ ID NOs: 1-446, SEQ ID NOs: 894-918, SEQ ID NOs: 945-1390, or SEQ ID NOs: 1392-1432) formulated together with one or more pharmaceutically acceptable excipients. Exemplary compositions provided herein include compositions comprising an inhibitor of STMN2 transcripts that include a cryptic exon, as described above, and one or more pharmaceutically acceptable excipients. Formulations include those suitable for oral, sublingual, intratracheal, intranasal, transdermal, pulmonary, intrathecal, intracisternal, parenteral (e.g., subcutaneous, intramuscular, intradermal, intraduodenal, or intravenous) or intralesional, administration, transmucosal (e.g., buccal, vaginal, and rectal), or for topical use. The most suitable form of administration in any given case will depend on the clinical symptoms, complications, or biochemical indicia of the state, disorder, disease, or condition that one is trying to prevent in a subject; the state, disorder, disease, or condition one is trying to prevent in a subject; and/or on the nature of the particular compound and/or the composition being used.

Inhibitors of STMN2 Transcripts that Include a Cryptic Exon

In certain embodiments, STMN2 levels (e.g., STMN2 mRNA or full length STMN2 protein levels) and/or activity (e.g., biological activity, for example, STMN2 activity) can be increased, restored, or stabilized using compounds or compositions that target a STMN2 gene product that includes a cryptic exon (for example, a STMN2 pre-mRNA).

In some embodiments, an inhibitor of STMN2 transcripts that include a cryptic exon can be, but is not limited to, nucleotide-based inhibitors of STMN2 (for example, STMN2 shRNAs, STMN2 siRNAs, STMN2 PNAs, STMN2 LNAs, 2′-O-methyl (2′OMe) STMN2 antisense oligonucleotide (AON), 2′-O-(2-methoxyethyl) (2′MOE) STMN2 AON, or STMN2 morpholino oligomers (e.g., phosphorodiamidate morpholino (PMO))), or compositions that include such compounds. In some embodiments an inhibitor of STMN2 is an antisense oligonucleotide (AON) comprising 2′OMe (e.g., an STMN2 AON comprising one or more 2′OMe modified sugar), MOE (e.g., an STMN2 AON comprising one or more MOE modified sugar (e.g., 2′-MOE)), PNA (e.g., a STMN2 AON comprising one or more N-(2-aminoethyl)-glycine units linked by amide bonds or carbonyl methylene linkage as repeating units in place of a sugar-phosphate backbone), LNA (e.g., a STMN2 AON comprising one or more locked ribose, and can be a mixture of 2′-deoxy nucleotides or 2′OMe nucleotides), c-ET (e.g., a STMN2 AON comprising one or more cET sugar), cMOE (e.g., a STMN2 AON comprising one or more cMOE sugar), morpholino oligomer (e.g., a STMN2 AON comprising a backbone comprising one or more PMO), deoxy-2′-fluoro nucleoside (e.g., a STMN2 AON comprising one or more 2′-fluoro-β-D-arabinonucleoside), ENA (e.g., a STMN2 AON comprising one or more ENA modified sugar), HNA (e.g., a STMN2 AON comprising one or more HNA modified sugar), or tcDNA (e.g., a STMN2 AON comprising one or more tcDNA modified sugar). In some embodiments, a STMN2 AON comprises one or more phosphorothioate linkage, phosphodiester linkage, phosphotriester linkage, methylphosphonate linkage, phosphoramidate linkage, phosphorodiamidate morpholino (PMO) linkage (“morpholino linkage”), peptide nucleic acid (PNA) linkage, or any combination of phosphorothioate linkage, phosphodiester linkage, a phosphotriester linkage, methylphosphonate linkage, phosphoramidate linkage, phosphorodiamidate morpholino (PMO) (morpholino) linkage, and PNA linkage. In some embodiments, a STMN2 AON comprises one or more phosphorothioate linkage, phosphodiester linkage, or a combination of phosphorothioate and phosphodiester linkages.

### STMN2 Antisense Therapeutics

Antisense therapeutics are a class of nucleic acid-based compounds that can be used to modulate a STMN2 mRNA or STMN2 transcript (for example, a STMN2 pre-mRNA comprising a cryptic exon). Antisense therapeutics may be single- or double-stranded deoxyribonucleic acid (DNA)-based, ribonucleic acid (RNA)-based, or DNA/RNA chemical analogue compounds. In general, antisense therapeutics are designed to include a nucleobase sequence that is complementary or nearly complementary to an mRNA or pre-mRNA sequence transcribed from a given gene in order to promote binding between the antisense therapeutic and the pre-mRNA or mRNA. In certain embodiments, antisense therapeutics act by binding to an mRNA or pre-mRNA, thereby inhibiting protein translation, altering pre-mRNA splicing into mature mRNA (e.g., by preventing appropriate proteins such as splicing activator proteins from binding), and/or causing destruction of mRNA. In certain embodiments, the antisense therapeutic nucleobase sequence is complementary to a portion of a targeted gene's or mRNA's sense sequence. In certain embodiments, STMN2 antisense therapeutics described herein are oligonucleotide-based compounds that include an oligonucleotide sequence complementary to a pre-mRNA sense, or a portion thereof. In certain embodiments, STMN2 antisense therapeutics described herein can also be nucleotide chemical analog-based compounds. Synthetic oligonucleotides as therapeutic agents has evolved into broad applications involving multiple modalities. These applications include ribozymes, small interfering RNA (siRNA), microRNA, aptamers, non-coding RNA, splicing modulation, targeting toxic repeats, gene editing, and immune modulations. The STMN2 oligonucleotides (STMN2 AONs) of the present disclosure prevent aberrant or mis-splicing by targeting a STMN2 transcript (e.g., STMN2 pre-mRNA (e.g., SEQ ID NO: 944)).

Antisense oligonucleotides (AONs) are short oligonucleotide-based sequences that include an oligonucleotide sequence complementary to a target RNA sequence. In certain embodiments, AONs are between 8 to 50 nucleotides in length, for example, 8, 10, 15, 20, 25, 30, 35, 40, 45, or 45 nucleotides in length. In certain embodiments, the AONs are 25 nucleotides in length. In certain embodiments, AONs may include chemically modified nucleosides (for example, 2′-O-methylated nucleosides or 2′-O-(2-methoxyethyl) nucleosides (2′-O-methoxyethylribonucleosides (2′-MOE))) as well as modified internucleoside linkages (for example, phosphorothioate linkages). In certain embodiments, STMN2 AONs described herein include oligonucleotide sequences that are complementary to STMN2 RNA sequences. In certain embodiments, STMN2 AONs described herein can include chemically modified nucleosides and modified internucleoside linkages (for example, phosphorothioate linkages).

Peptide nucleic acids (PNAs) are short, artificially synthesized polymers with a structure that mimics DNA or RNA. PNAs include a backbone composed of repeating N-(2-aminoethyl)-glycine units linked by peptide bonds. In certain embodiments, STMN2 PNAs described herein can be used as antisense therapeutics that bind to STMN2 RNA sequences with high specificity and increase, restore, and/or stabilize STMN2 levels (e.g., STMN2 mRNA or protein levels) and/or activity (e.g., biological activity, for example, STMN2 activity).

Locked nucleic acids (LNAs) are oligonucleotide sequences that include one or more modified RNA nucleotides in which the ribose moiety is modified with an extra bridge connecting the 2′ oxygen and 4′ carbon. LNAs are believed to have higher Tm's than analogous oligonucleotide sequences. In certain embodiments, STMN2 LNAs described herein can be used as antisense therapeutics that bind to STMN2 RNA sequences with high specificity and repress premature polyadenylation of STMN2 pre-mRNA, and increase, restore, and/or stabilize STMN2 levels (e.g., STMN2 mRNA or protein levels) and/or activity (e.g., biological activity, for example, STMN2 activity).

Morpholino oligomers are oligonucleotide compounds that include DNA bases attached to a backbone of methylenemorpholine rings linked through phosphorodiamidate groups. In certain embodiments, morpholino oligomers of the present invention can be designed to bind to specific STMN2 pre-mRNA sequence of interest, thereby repressing premature polyadenylation of the pre-mRNA, and increase, restore, and/or stabilize STMN2 levels (e.g., STMN2 mRNA or protein levels) and/or activity (e.g., biological activity, for example, STMN2 activity). In certain embodiments, STMN2 morpholino oligomers described herein can be used as antisense therapeutics that bind to STMN2 pre-mRNA sequences with high specificity and repress premature polyadenylation of STMN2 pre-mRNA, and increase, restore, and/or stabilize STMN2 levels (e.g., STMN2 mRNA or protein levels) and/or activity (e.g., biological activity, for example, STMN2 activity). In certain embodiments, STMN2 morpholino oligomers described herein can also be used to bind STMN2 pre-mRNA sequences, altering STMN2 pre-mRNA splicing and STMN2 gene expression, and increase, restore, and/or stabilize STMN2 levels (e.g., STMN2 mRNA or protein levels) and/or activity (e.g., biological activity, for example, STMN2 activity).

In some embodiments, STMN2 antisense therapeutics include a STMN2 AON comprising 2′OMe (e.g., an STMN2 AON comprising one or more 2′OMe modified sugar), MOE (e.g., an STMN2 AON comprising one or more MOE modified sugar (e.g., 2′-MOE)), PNA (e.g., a STMN2 AON comprising one or more N-(2-aminoethyl)-glycine units linked by amide bonds or carbonyl methylene linkage as repeating units in place of a sugar-phosphate backbone), LNA (e.g., a STMN2 AON comprising one or more locked ribose, and can be a mixture of 2′-deoxy nucleotides or 2′OMe nucleotides), c-ET (e.g., a STMN2 AON comprising one or more cET sugar), cMOE (e.g., a STMN2 AON comprising one or more cMOE sugar), morpholino oligomer (e.g., a STMN2 AON comprising a backbone comprising one or more PMO), deoxy-2′-fluoro nucleoside (e.g., a STMN2 AON comprising one or more 2′-fluoro-β-D-arabinonucleoside), ENA (e.g., a STMN2 AON comprising one or more ENA modified sugar), HNA (e.g., a STMN2 AON comprising one or more HNA modified sugar), or tcDNA (e.g., a STMN2 AON comprising one or more tcDNA modified sugar). In some embodiments, a STMN2 AON comprises one or more phosphorothioate linkage, phosphodiester linkage, phosphotriester linkage, methylphosphonate linkage, phosphoramidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a STMN2 AON comprises one or more phosphorothioate linkage, phosphodiester linkage, or a combination of phosphorothioate and phosphodiester linkages.

### STMN2 Antisense Oligonucleotides

In certain embodiments, a STMN2 antisense oligonucleotide, such as disclosed herein, may be an oligonucleotide sequence of 5 to 100 nucleotides in length, for example, 10 to 40 nucleotides in length, for example, 14 to 40 nucleotides in length, 10 to 30 nucleotides in length, for example, 14 to 30 nucleotides in length, for example, 14 to 25 or 15 to 22 nucleotides in length, or 18, 19, 20, 21, 22, 23, 24, or 25 nucleotides in length. In certain embodiments, the AONs are 25 nucleotides in length. In certain embodiments, STMN2 antisense oligonucleotides (AONs) described herein are short synthetic oligonucleotide sequence complementary to a STMN2 transcript (e.g., pre-mRNA), a portion of a STMN2 transcript, or a STMN2 gene sequence.

In some embodiments, a STMN2 AON includes a nucleobase sequence that is 80%, 85%, 90%, 95%, or 100% complementary to the STMN2 transcript (e.g., STMN2 pre-mRNA) that includes a cryptic exon. In some embodiments, the nucleobase sequence of the STMN2 antisense oligonucleotide is 10, 11, 12, 13, 14, 15, 16, 17, 18, 19, 20, 21, 22, 23, 24, or 25 contiguous nucleobases that are 80%, 85%, 90%, 95%, or 100% complementary to an equal length portion of nucleobases in a portion of the STMN2 transcript that includes a cryptic exon.

AON binding specificity can be assessed via measurement of parameters such as dissociation constant, melting temperature (Tm), or other criteria such as changes in protein or RNA expression levels or other assays that measure STMN2 activity or expression.

In some embodiments, a STMN2 AON can include a non-duplexed oligonucleotide. In some embodiments, a STMN2 AON can include a duplex of two oligonucleotides where the first oligonucleotide includes a nucleobase sequence that is completely or almost completely complementary to a STMN2 pre-mRNA sequence and the second oligonucleotide includes a nucleobase sequence that is complementary to the nucleobase sequence of the first oligonucleotide.

In some embodiments, a STMN2 AON can target STMN2 pre-mRNAs that include a cryptic exon produced from STMN2 genes of one or more species. For example, a STMN2 AON can target a STMN2 pre-mRNA, which includes a cryptic exon, of a mammalian STMN2 gene, for example, a human (i.e., Homo sapiens) STMN2 gene. In particular embodiments, the STMN2 AON targets a human STMN2 pre-mRNA, which includes a cryptic exon. In some embodiments, the STMN2 AON includes a nucleobase sequence that is complementary to a nucleobase sequence of a STMN2 gene or a STMN2 pre-mRNA, which includes a cryptic exon, or a portion thereof.

STMN2 AONs described herein include antisense oligonucleotides comprising the oligonucleotide sequences listed in Table 1 below:

TABLE 1
STMN2 AON Sequences

| SEQ | | | |
|---|---|---|---|
| ID | AON Sequence* | | Target Sequence |
| NO: | (5′ → 3′) | Region | (5′ → 3′) |
| 1 | GGAGGGATACCTGTATATTACAAGT | | ACTTGTAATATACAGGTATCCCTCC |
| | | | SEQ ID NO: 448 |
| 2 | AGGAGGGATACCTGTATATTACAAG | | CTTGTAATATACAGGTATCCCTCCT |
| | | | SEQ ID NO: 449 |
| 3 | CAGGAGGGATACCTGTATATTACAA | | TTGTAATATACAGGTATCCCTCCTG |
| | | | SEQ ID NO: 450 |
| 4 | CCAGGAGGGATACCTGTATATTACA | | TGTAATATACAGGTATCCCTCCTGG |
| | | | SEQ ID NO: 451 |
| 5 | ACCAGGAGGGATACCTGTATATTAC | | GTAATATACAGGTATCCCTCCTGGT |
| | | | SEQ ID NO: 452 |
| 6 | TACCAGGAGGGATACCTGTATATTA | | TAATATACAGGTATCCCTCCTGGTA |
| | | | SEQ ID NO: 453 |
| 7 | TTACCAGGAGGGATACCTGTATATT | | AATATACAGGTATCCCTCCTGGTAA |
| | | | SEQ ID NO: 454 |
| 8 | CTTACCAGGAGGGATACCTGTATAT | | ATATACAGGTATCCCTCCTGGTAAG |
| | | | SEQ ID NO: 455 |
| 9 | GCTTACCAGGAGGGATACCTGTATA | | TATACAGGTATCCCTCCTGGTAAGC |
| | | | SEQ ID NO: 456 |
| 10 | AGCTTACCAGGAGGGATACCTGTAT | | ATACAGGTATCCCTCCTGGTAAGCT |
| | | | SEQ ID NO: 457 |
| 11 | GAGCTTACCAGGAGGGATACCTGTA | | TACAGGTATCCCTCCTGGTAAGCTC |
| | | | SEQ ID NO: 458 |
| 12 | AGAGCTTACCAGGAGGGATACCTGT | | ACAGGTATCCCTCCTGGTAAGCTCT |
| | | | SEQ ID NO: 459 |
| 13 | CAGAGCTTACCAGGAGGGATACCTG | | CAGGTATCCCTCCTGGTAAGCTCTG |
| | | | SEQ ID NO: 460 |
| 14 | CCAGAGCTTACCAGGAGGGATACCT | | AGGTATCCCTCCTGGTAAGCTCTGG |
| | | | SEQ ID NO: 461 |
| 15 | ACCAGAGCTTACCAGGAGGGATACC | | GGTATCCCTCCTGGTAAGCTCTGGT |
| | | | SEQ ID NO: 462 |
| 16 | TACCAGAGCTTACCAGGAGGGATAC | | GTATCCCTCCTGGTAAGCTCTGGTA |
| | | | SEQ ID NO: 463 |
| 17 | ATACCAGAGCTTACCAGGAGGGATA | | TATCCCTCCTGGTAAGCTCTGGTAT |
| | | | SEQ ID NO: 464 |
| 18 | AATACCAGAGCTTACCAGGAGGGAT | | ATCCCTCCTGGTAAGCTCTGGTATT |
| | | | SEQ ID NO: 465 |
| 19 | TAATACCAGAGCTTACCAGGAGGGA | | TCCCTCCTGGTAAGCTCTGGTATTA |
| | | | SEQ ID NO: 466 |
| 20 | ATAATACCAGAGCTTACCAGGAGGG | | CCCTCCTGGTAAGCTCTGGTATTAT |
| | | | SEQ ID NO: 467 |
| 21 | CATAATACCAGAGCTTACCAGGAGG | | CCTCCTGGTAAGCTCTGGTATTATG |
| | | | SEQ ID NO: 468 |
| 22 | ACATAATACCAGAGCTTACCAGGAG | | CTCCTGGTAAGCTCTGGTATTATGT |
| | | | SEQ ID NO: 469 |
| 23 | GACATAATACCAGAGCTTACCAGGA | | TCCTGGTAAGCTCTGGTATTATGTC |
| | | | SEQ ID NO: 470 |
| 24 | AGACATAATACCAGAGCTTACCAGG | | CCTGGTAAGCTCTGGTATTATGTCT |
| | | | SEQ ID NO: 471 |
| 25 | AAGACATAATACCAGAGCTTACCAG | | CTGGTAAGCTCTGGTATTATGTCTT |
| | | | SEQ ID NO: 472 |
| 26 | TAAGACATAATACCAGAGCTTACCA | | TGGTAAGCTCTGGTATTATGTCTTA |
| | | | SEQ ID NO: 473 |
| 27 | TTAAGACATAATACCAGAGCTTACC | | GGTAAGCTCTGGTATTATGTCTTAA |
| | | | SEQ ID NO: 474 |
| 28 | GTTAAGACATAATACCAGAGCTTAC | | GTAAGCTCTGGTATTATGTCTTAAC |
| | | | SEQ ID NO: 475 |
| 29 | TGTTAAGACATAATACCAGAGCTTA | | TAAGCTCTGGTATTATGTCTTAACA |
| | | | SEQ ID NO: 476 |
| 30 | ATGTTAAGACATAATACCAGAGCTT | branch | AAGCTCTGGTATTATGTCTTAACAT |
| | | point 1 | SEQ ID NO: 477 |
| 31 | AATGTTAAGACATAATACCAGAGCT | branch | AGCTCTGGTATTATGTCTTAACATT |
| | | point 1 | SEQ ID NO: 478 |
| 32 | AAATGTTAAGACATAATACCAGAGC | branch | GCTCTGGTATTATGTCTTAACATTT |
| | | point 1 | SEQ ID NO: 479 |
| 33 | AAAATGTTAAGACATAATACCAGAG | branch | CTCTGGTATTATGTCTTAACATTTT |
| | | point 1 | SEQ ID NO: 480 |
| 34 | AAAAATGTTAAGACATAATACCAGA | branch | TCTGGTATTATGTCTTAACATTTTT |
| | | point 1 | SEQ ID NO: 481 |
| 35 | TAAAAATGTTAAGACATAATACCAG | branch | CTGGTATTATGTCTTAACATTTTTA |
| | | point 1 | SEQ ID NO: 482 |
| 36 | TTAAAAATGTTAAGACATAATACCA | branch | TGGTATTATGTCTTAACATTTTTAA |
| | | point 1 | SEQ ID NO: 483 |
| 37 | TTTAAAAATGTTAAGACATAATACC | branch | GGTATTATGTCTTAACATTTTTAAA |
| | | point 1 | SEQ ID NO: 484 |
| 38 | ATTTAAAAATGTTAAGACATAATAC | branch | GTATTATGTCTTAACATTTTTAAAT |
| | | point 1 | SEQ ID NO: 485 |
| 39 | GATTTAAAAATGTTAAGACATAATA | branch | TATTATGTCTTAACATTTTTAAATC |
| | | point 1 | SEQ ID NO: 486 |
| 40 | AGATTTAAAAATGTTAAGACATAAT | branch | ATTATGTCTTAACATTTTTAAATCT |
| | | point 1 | SEQ ID NO: 487 |
| 41 | TAGATTTAAAAATGTTAAGACATAA | branch | TTATGTCTTAACATTTTTAAATCTA |
| | | point 1 | SEQ ID NO: 488 |
| 42 | ATAGATTTAAAAATGTTAAGACATA | branch | TATGTCTTAACATTTTTAAATCTAT |
| | | point 1 | SEQ ID NO: 489 |
| 43 | CATAGATTTAAAAATGTTAAGACAT | branch | ATGTCTTAACATTTTTAAATCTATG |
| | | point 1 | SEQ ID NO: 490 |
| 44 | CCATAGATTTAAAAATGTTAAGACA | branch | TGTCTTAACATTTTTAAATCTATGG |
| | | point 1 | SEQ ID NO: 491 |
| 45 | ACCATAGATTTAAAAATGTTAAGAC | branch | GTCTTAACATTTTTAAATCTATGGT |
| | | point 1 | SEQ ID NO: 492 |
| 46 | TACCATAGATTTAAAAATGTTAAGA | branch | TCTTAACATTTTTAAATCTATGGTA |
| | | point 1 | SEQ ID NO: 493 |
| 47 | TTACCATAGATTTAAAAATGTTAAG | | CTTAACATTTTTAAATCTATGGTAA |
| | | | SEQ ID NO: 494 |
| 48 | ATTACCATAGATTTAAAAATGTTAA | | TTAACATTTTTAAATCTATGGTAAT |
| | | | SEQ ID NO: 495 |
| 49 | GATTACCATAGATTTAAAAATGTTA | | TAACATTTTTAAATCTATGGTAATC |
| | | | SEQ ID NO: 496 |
| 50 | AGATTACCATAGATTTAAAAATGTT | Branch | AACATTTTTAAATCTATGGTAATCT |
| | | point 2 | SEQ ID NO: 497 |
| 51 | AAGATTACCATAGATTTAAAAATGT | Branch | ACATTTTTAAATCTATGGTAATCTT |
| | | point 2 | SEQ ID NO: 498 |
| 52 | AAAGATTACCATAGATTTAAAAATG | Branch | CATTTTTAAATCTATGGTAATCTTT |
| | | point 2 | SEQ ID NO: 499 |
| 53 | TAAAGATTACCATAGATTTAAAAAT | Branch | ATTTTTAAATCTATGGTAATCTTTA |
| | | point 2 | SEQ ID NO: 500 |
| 54 | GTAAAGATTACCATAGATTTAAAAA | Branch | TTTTTAAATCTATGGTAATCTTTAC |
| | | point 2 | SEQ ID NO: 501 |
| 55 | TGTAAAGATTACCATAGATTTAAAA | Branch | TTTTAAATCTATGGTAATCTTTACA |
| | | point 2 | SEQ ID NO: 502 |
| 56 | TTGTAAAGATTACCATAGATTTAAA | Branch | TTTAAATCTATGGTAATCTTTACAA |
| | | point 2 | SEQ ID NO: 503 |
| 57 | TTTGTAAAGATTACCATAGATTTAA | Branch | TTAAATCTATGGTAATCTTTACAAA |
| | | point 2 | SEQ ID NO: 504 |
| 58 | TTTTGTAAAGATTACCATAGATTTA | Branch | TAAATCTATGGTAATCTTTACAAAA |
| | | point 2 | SEQ ID NO: 505 |
| 59 | ATTTTGTAAAGATTACCATAGATTT | Branch | AAATCTATGGTAATCTTTACAAAAT |
| | | point 2 | SEQ ID NO: 506 |
| 60 | TATTTTGTAAAGATTACCATAGATT | Branch | AATCTATGGTAATCTTTACAAAATA |
| | | point 2 | SEQ ID NO: 507 |
| 61 | ATATTTTGTAAAGATTACCATAGAT | Branch | ATCTATGGTAATCTTTACAAAATAT |
| | | point 2 | SEQ ID NO: 508 |
| 62 | AATATTTTGTAAAGATTACCATAGA | Branch | TCTATGGTAATCTTTACAAAATATT |
| | | point 2 | SEQ ID NO: 509 |
| 63 | AAATATTTTGTAAAGATTACCATAG | Branch | CTATGGTAATCTTTACAAAATATTT |
| | | point 2 | SEQ ID NO: 510 |
| 64 | AAAATATTTTGTAAAGATTACCATA | Branch | TATGGTAATCTTTACAAAATATTTT |
| | | point 2 | SEQ ID NO: 511 |
| 65 | TAAAATATTTTGTAAAGATTACCAT | Branch | ATGGTAATCTTTACAAAATATTTTA |
| | | point 2 | SEQ ID NO: 512 |
| 66 | GTAAAATATTTTGTAAAGATTACCA | Branch | TGGTAATCTTTACAAAATATTTTAC |
| | | point 2 | SEQ ID NO: 513 |
| 67 | AGTAAAATATTTTGTAAAGATTACC | | GGTAATCTTTACAAAATATTTTACT |
| | | | SEQ ID NO: 514 |
| 68 | AAGTAAAATATTTTGTAAAGATTAC | | GTAATCTTTACAAAATATTTTACTT |
| | | | SEQ ID NO: 515 |
| 69 | GAAGTAAAATATTTTGTAAAGATTA | | TAATCTTTACAAAATATTTTACTTC |
| | | | SEQ ID NO: 516 |
| 70 | GGAAGTAAAATATTTTGTAAAGATT | | AATCTTTACAAAATATTTTACTTCC |
| | | | SEQ ID NO: 517 |
| 71 | CGGAAGTAAAATATTTTGTAAAGAT | | ATCTTTACAAAATATTTTACTTCCG |
| | | | SEQ ID NO: 518 |
| 72 | TCGGAAGTAAAATATTTTGTAAAGA | | TCTTTACAAAATATTTTACTTCCGA |
| | | | SEQ ID NO: 519 |
| 73 | TTCGGAAGTAAAATATTTTGTAAAG | | CTTTACAAAATATTTTACTTCCGAA |
| | | | SEQ ID NO: 520 |
| 74 | GTTCGGAAGTAAAATATTTTGTAAA | | TTTACAAAATATTTTACTTCCGAAC |
| | | | SEQ ID NO: 521 |
| 75 | AGTTCGGAAGTAAAATATTTTGTAA | | TTACAAAATATTTTACTTCCGAACT |
| | | | SEQ ID NO: 522 |
| 76 | GAGTTCGGAAGTAAAATATTTTGTA | | TACAAAATATTTTACTTCCGAACTC |
| | | | SEQ ID NO: 523 |
| 77 | TGAGTTCGGAAGTAAAATATTTTGT | | ACAAAATATTTTACTTCCGAACTCA |
| | | | SEQ ID NO: 524 |
| 78 | ATGAGTTCGGAAGTAAAATATTTTG | | CAAAATATTTTACTTCCGAACTCAT |
| | | | SEQ ID NO: 525 |
| 79 | TATGAGTTCGGAAGTAAAATATTTT | | AAAATATTTTACTTCCGAACTCATA |
| | | | SEQ ID NO: 526 |
| 80 | ATATGAGTTCGGAAGTAAAATATTT | | AAATATTTTACTTCCGAACTCATAT |
| | | | SEQ ID NO: 527 |
| 81 | TATATGAGTTCGGAAGTAAAATATT | | AATATTTTACTTCCGAACTCATATA |
| | | | SEQ ID NO: 528 |
| 82 | GTATATGAGTTCGGAAGTAAAATAT | | ATATTTTACTTCCGAACTCATATAC |
| | | | SEQ ID NO: 529 |
| 83 | GGTATATGAGTTCGGAAGTAAAATA | | TATTTTACTTCCGAACTCATATACC |
| | | | SEQ ID NO: 530 |
| 84 | AGGTATATGAGTTCGGAAGTAAAAT | | ATTTTACTTCCGAACTCATATACCT |
| | | | SEQ ID NO: 531 |
| 85 | CAGGTATATGAGTTCGGAAGTAAAA | | TTTTACTTCCGAACTCATATACCTG |
| | | | SEQ ID NO: 532 |
| 86 | CCAGGTATATGAGTTCGGAAGTAAA | | TTTACTTCCGAACTCATATACCTGG |
| | | | SEQ ID NO: 533 |
| 87 | CCCAGGTATATGAGTTCGGAAGTAA | | TTACTTCCGAACTCATATACCTGGG |
| | | | SEQ ID NO: 534 |
| 88 | CCCCAGGTATATGAGTTCGGAAGTA | | TACTTCCGAACTCATATACCTGGGG |
| | | | SEQ ID NO: 535 |
| 89 | TCCCCAGGTATATGAGTTCGGAAGT | | ACTTCCGAACTCATATACCTGGGGA |
| | | | SEQ ID NO: 536 |
| 90 | ATCCCCAGGTATATGAGTTCGGAAG | | CTTCCGAACTCATATACCTGGGGAT |
| | | | SEQ ID NO: 537 |
| 91 | AATCCCCAGGTATATGAGTTCGGAA | | TTCCGAACTCATATACCTGGGGATT |
| | | | SEQ ID NO: 538 |
| 92 | AAATCCCCAGGTATATGAGTTCGGA | | TCCGAACTCATATACCTGGGGATTT |
| | | | SEQ ID NO: 539 |
| 93 | AAAATCCCCAGGTATATGAGTTCGG | | CCGAACTCATATACCTGGGGATTTT |
| | | | SEQ ID NO: 540 |
| 94 | TAAAATCCCCAGGTATATGAGTTCG | | CGAACTCATATACCTGGGGATTTTA |
| | | | SEQ ID NO: 541 |
| 95 | ATAAAATCCCCAGGTATATGAGTTC | | GAACTCATATACCTGGGGATTTTAT |
| | | | SEQ ID NO: 542 |
| 96 | AATAAAATCCCCAGGTATATGAGTT | | AACTCATATACCTGGGGATTTTATT |
| | | | SEQ ID NO: 543 |
| 97 | TAATAAAATCCCCAGGTATATGAGT | | ACTCATATACCTGGGGATTTTATTA |
| | | | SEQ ID NO: 544 |
| 98 | GTAATAAAATCCCCAGGTATATGAG | | CTCATATACCTGGGGATTTTATTAC |
| | | | SEQ ID NO: 545 |
| 99 | AGTAATAAAATCCCCAGGTATATGA | | TCATATACCTGGGGATTTTATTACT |
| | | | SEQ ID NO: 546 |
| 100 | GAGTAATAAAATCCCCAGGTATATG | | CATATACCTGGGGATTTTATTACTC |
| | | | SEQ ID NO: 547 |
| 101 | AGAGTAATAAAATCCCCAGGTATAT | | ATATACCTGGGGATTTTATTACTCT |
| | | | SEQ ID NO: 548 |
| 102 | CAGAGTAATAAAATCCCCAGGTATA | | TATACCTGGGGATTTTATTACTCTG |
| | | | SEQ ID NO: 549 |
| 103 | CCAGAGTAATAAAATCCCCAGGTAT | | ATACCTGGGGATTTTATTACTCTGG |
| | | | SEQ ID NO: 550 |
| 104 | CCCAGAGTAATAAAATCCCCAGGTA | | TACCTGGGGATTTTATTACTCTGGG |
| | | | SEQ ID NO: 551 |
| 105 | TCCCAGAGTAATAAAATCCCCAGGT | | ACCTGGGGATTTTATTACTCTGGGA |
| | | | SEQ ID NO: 552 |
| 106 | TTCCCAGAGTAATAAAATCCCCAGG | | CCTGGGGATTTTATTACTCTGGGAA |
| | | | SEQ ID NO: 553 |
| 107 | ATTCCCAGAGTAATAAAATCCCCAG | | CTGGGGATTTTATTACTCTGGGAAT |
| | | | SEQ ID NO: 554 |
| 108 | AATTCCCAGAGTAATAAAATCCCCA | | TGGGGATTTTATTACTCTGGGAATT |
| | | | SEQ ID NO: 555 |
| 109 | TAATTCCCAGAGTAATAAAATCCCC | | GGGGATTTTATTACTCTGGGAATTA |
| | | | SEQ ID NO: 556 |
| 110 | ATAATTCCCAGAGTAATAAAATCCC | | GGGATTTTATTACTCTGGGAATTAT |
| | | | SEQ ID NO: 557 |
| ill | CATAATTCCCAGAGTAATAAAATCC | | GGATTTTATTACTCTGGGAATTATG |
| | | | SEQ ID NO: 558 |
| 112 | ACATAATTCCCAGAGTAATAAAATC | | GATTTTATTACTCTGGGAATTATGT |
| | | | SEQ ID NO: 559 |
| 113 | CACATAATTCCCAGAGTAATAAAAT | | ATTTTATTACTCTGGGAATTATGTG |
| | | | SEQ ID NO: 560 |
| 114 | ACACATAATTCCCAGAGTAATAAAA | | TTTTATTACTCTGGGAATTATGTGT |
| | | | SEQ ID NO: 561 |
| 115 | AACACATAATTCCCAGAGTAATAAA | | TTTATTACTCTGGGAATTATGTGTT |
| | | | SEQ ID NO: 562 |
| 116 | GAACACATAATTCCCAGAGTAATAA | | TTATTACTCTGGGAATTATGTGTTC |
| | | | SEQ ID NO: 563 |
| 117 | AGAACACATAATTCCCAGAGTAATA | | TATTACTCTGGGAATTATGTGTTCT |
| | | | SEQ ID NO: 564 |
| 118 | CAGAACACATAATTCCCAGAGTAAT | | ATTACTCTGGGAATTATGTGTTCTG |
| | | | SEQ ID NO: 565 |
| 119 | GCAGAACACATAATTCCCAGAGTAA | | TTACTCTGGGAATTATGTGTTCTGC |
| | | | SEQ ID NO: 566 |
| 120 | GGCAGAACACATAATTCCCAGAGTA | | TACTCTGGGAATTATGTGTTCTGCC |
| | | | SEQ ID NO: 567 |
| 121 | GGGCAGAACACATAATTCCCAGAGT | | ACTCTGGGAATTATGTGTTCTGCCC |
| | | | SEQ ID NO: 568 |
| 122 | GGGGCAGAACACATAATTCCCAGAG | | CTCTGGGAATTATGTGTTCTGCCCC |
| | | | SEQ ID NO: 569 |
| 123 | TGGGGCAGAACACATAATTCCCAGA | | TCTGGGAATTATGTGTTCTGCCCCA |
| | | | SEQ ID NO: 570 |
| 124 | ATGGGGCAGAACACATAATTCCCAG | | CTGGGAATTATGTGTTCTGCCCCAT |
| | | | SEQ ID NO: 571 |
| 125 | GATGGGGCAGAACACATAATTCCCA | | TGGGAATTATGTGTTCTGCCCCATC |
| | | | SEQ ID NO: 572 |
| 126 | TGATGGGGCAGAACACATAATTCCC | | GGGAATTATGTGTTCTGCCCCATCA |
| | | | SEQ ID NO: 573 |
| 127 | GTGATGGGGCAGAACACATAATTCC | | GGAATTATGTGTTCTGCCCCATCAC |
| | | | SEQ ID NO: 574 |
| 128 | AGTGATGGGGCAGAACACATAATTC | | GAATTATGTGTTCTGCCCCATCACT |
| | | | SEQ ID NO: 575 |
| 129 | GAGTGATGGGGCAGAACACATAATT | Branch | AATTATGTGTTCTGCCCCATCACTC |
| | | point 3 | SEQ ID NO: 576 |
| 130 | AGAGTGATGGGGCAGAACACATAAT | Branch | ATTATGTGTTCTGCCCCATCACTCT |
| | | point 3 | SEQ ID NO: 577 |
| 131 | GAGAGTGATGGGGCAGAACACATAA | Branch | TTATGTGTTCTGCCCCATCACTCTC |
| | | point 3 | SEQ ID NO: 578 |
| 132 | AGAGAGTGATGGGGCAGAACACATA | Branch | TATGTGTTCTGCCCCATCACTCTCT |
| | | point 3 | SEQ ID NO: 579 |
| 133 | GAGAGAGTGATGGGGCAGAACACAT | Branch | ATGTGTTCTGCCCCATCACTCTCTC |
| | | point 3 | SEQ ID NO: 580 |
| 134 | AGAGAGAGTGATGGGGCAGAACACA | Branch | TGTGTTCTGCCCCATCACTCTCTCT |
| | | point 3 | SEQ ID NO: 581 |
| 135 | AAGAGAGAGTGATGGGGCAGAACAC | Branch | GTGTTCTGCCCCATCACTCTCTCTT |
| | | point 3 | SEQ ID NO: 582 |
| 136 | TAAGAGAGAGTGATGGGGCAGAACA | Branch | TGTTCTGCCCCATCACTCTCTCTTA |
| | | point 3 | SEQ ID NO: 583 |
| 137 | TTAAGAGAGAGTGATGGGGCAGAAC | Branch | GTTCTGCCCCATCACTCTCTCTTAA |
| | | point 3 | SEQ ID NO: 584 |
| 138 | ATTAAGAGAGAGTGATGGGGCAGAA | Branch | TTCTGCCCCATCACTCTCTCTTAAT |
| | | point 3 | SEQ ID NO: 585 |
| 139 | AATTAAGAGAGAGTGATGGGGCAGA | Branch | TCTGCCCCATCACTCTCTCTTAATT |
| | | point 3 | SEQ ID NO: 586 |
| 140 | CAATTAAGAGAGAGTGATGGGGCAG | Branch | CTGCCCCATCACTCTCTCTTAATTG |
| | | point 3 | SEQ ID NO: 587 |
| 141 | CCAATTAAGAGAGAGTGATGGGGCA | Branch | TGCCCCATCACTCTCTCTTAATTGG |
| | | point 3 | SEQ ID NO: 588 |
| 142 | TCCAATTAAGAGAGAGTGATGGGGC | Branch | GCCCCATCACTCTCTCTTAATTGGA |
| | | point 3 | SEQ ID NO: 589 |
| 143 | ATCCAATTAAGAGAGAGTGATGGGG | Branch | CCCCATCACTCTCTCTTAATTGGAT |
| | | point 3 | SEQ ID NO: 590 |
| 144 | AATCCAATTAAGAGAGAGTGATGGG | Branch | CCCATCACTCTCTCTTAATTGGATT |
| | | point 3 | SEQ ID NO: 591 |
| 145 | AAATCCAATTAAGAGAGAGTGATGG | Branch | CCATCACTCTCTCTTAATTGGATTT |
| | | point 3 | SEQ ID NO: 592 |
| 146 | AAAATCCAATTAAGAGAGAGTGATG | | CATCACTCTCTCTTAATTGGATTTT |
| | | | SEQ ID NO: 593 |
| 147 | AAAAATCCAATTAAGAGAGAGTGAT | | ATCACTCTCTCTTAATTGGATTTTT |
| | | | SEQ ID NO: 594 |
| 148 | TAAAAATCCAATTAAGAGAGAGTGA | | TCACTCTCTCTTAATTGGATTTTTA |
| | | | SEQ ID NO: 595 |
| 149 | TTAAAAATCCAATTAAGAGAGAGTG | | CACTCTCTCTTAATTGGATTTTTAA |
| | | | SEQ ID NO: 596 |
| 150 | TTTAAAAATCCAATTAAGAGAGAGT | | ACTCTCTCTTAATTGGATTTTTAAA |
| | | | SEQ ID NO: 597 |
| 151 | TTTTAAAAATCCAATTAAGAGAGAG | | CTCTCTCTTAATTGGATTTTTAAAA |
| | | | SEQ ID NO: 598 |
| 152 | ATTTTAAAAATCCAATTAAGAGAGA | | TCTCTCTTAATTGGATTTTTAAAAT |
| | | | SEQ ID NO: 599 |
| 153 | AATTTTAAAAATCCAATTAAGAGAG | | CTCTCTTAATTGGATTTTTAAAATT |
| | | | SEQ ID NO: 600 |
| 154 | TAATTTTAAAAATCCAATTAAGAGA | | TCTCTTAATTGGATTTTTAAAATTA |
| | | | SEQ ID NO: 601 |
| 155 | ATAATTTTAAAAATCCAATTAAGAG | | CTCTTAATTGGATTTTTAAAATTAT |
| | | | SEQ ID NO: 602 |
| 156 | TATAATTTTAAAAATCCAATTAAGA | | TCTTAATTGGATTTTTAAAATTATA |
| | | | SEQ ID NO: 603 |
| 157 | ATATAATTTTAAAAATCCAATTAAG | | CTTAATTGGATTTTTAAAATTATAT |
| | | | SEQ ID NO: 604 |
| 158 | AATATAATTTTAAAAATCCAATTAA | | TTAATTGGATTTTTAAAATTATATT |
| | | | SEQ ID NO: 605 |
| 159 | GAATATAATTTTAAAAATCCAATTA | | TAATTGGATTTTTAAAATTATATTC |
| | | | SEQ ID NO: 606 |
| 160 | TGAATATAATTTTAAAAATCCAATT | | AATTGGATTTTTAAAATTATATTCA |
| | | | SEQ ID NO: 607 |
| 161 | ATGAATATAATTTTAAAAATCCAAT | | ATTGGATTTTTAAAATTATATTCAT |
| | | | SEQ ID NO: 608 |
| 162 | TATGAATATAATTTTAAAAATCCAA | | TTGGATTTTTAAAATTATATTCATA |
| | | | SEQ ID NO: 609 |
| 163 | ATATGAATATAATTTTAAAAATCCA | | TGGATTTTTAAAATTATATTCATAT |
| | | | SEQ ID NO: 610 |
| 164 | AATATGAATATAATTTTAAAAATCC | | GGATTTTTAAAATTATATTCATATT |
| | | | SEQ ID NO: 611 |
| 165 | CAATATGAATATAATTTTAAAAATC | | GATTTTTAAAATTATATTCATATTG |
| | | | SEQ ID NO: 612 |
| 166 | GCAATATGAATATAATTTTAAAAAT | | ATTTTTAAAATTATATTCATATTGC |
| | | | SEQ ID NO: 613 |
| 167 | TGCAATATGAATATAATTTTAAAAA | | TTTTTAAAATTATATTCATATTGCA |
| | | | SEQ ID NO: 614 |
| 168 | CTGCAATATGAATATAATTTTAAAA | | TTTTAAAATTATATTCATATTGCAG |
| | | | SEQ ID NO: 615 |
| 169 | CCTGCAATATGAATATAATTTTAAA | | TTTAAAATTATATTCATATTGCAGG |
| | | | SEQ ID NO: 616 |
| 170 | TCCTGCAATATGAATATAATTTTAA | | TTAAAATTATATTCATATTGCAGGA |
| | | | SEQ ID NO: 617 |
| 171 | GTCCTGCAATATGAATATAATTTTA | Acceptor | TAAAATTATATTCATATTGCAGGAC |
| | | site | SEQ ID NO: 618 |
| 172 | AGTCCTGCAATATGAATATAATTTT | Acceptor | AAAATTATATTCATATTGCAGGACT |
| | | site | SEQ ID NO: 619 |
| 173 | GAGTCCTGCAATATGAATATAATTT | Acceptor | AAATTATATTCATATTGCAGGACTC |
| | | site | SEQ ID NO: 620 |
| 174 | CGAGTCCTGCAATATGAATATAATT | Acceptor | AATTATATTCATATTGCAGGACTCG |
| | | site | SEQ ID NO: 621 |
| 175 | CCGAGTCCTGCAATATGAATATAAT | Acceptor | ATTATATTCATATTGCAGGACTCGG |
| | | site | SEQ ID NO: 622 |
| 176 | GCCGAGTCCTGCAATATGAATATAA | Acceptor | TTATATTCATATTGCAGGACTCGGC |
| | | site | SEQ ID NO: 623 |
| 177 | TGCCGAGTCCTGCAATATGAATATA | Acceptor | TATATTCATATTGCAGGACTCGGCA |
| | | site | SEQ ID NO: 624 |
| 178 | CTGCCGAGTCCTGCAATATGAATAT | Acceptor | ATATTCATATTGCAGGACTCGGCAG |
| | | site | SEQ ID NO: 625 |
| 179 | TCTGCCGAGTCCTGCAATATGAATA | Acceptor | TATTCATATTGCAGGACTCGGCAGA |
| | | site | SEQ ID NO: 626 |
| 180 | TTCTGCCGAGTCCTGCAATATGAAT | Acceptor | ATTCATATTGCAGGACTCGGCAGAA |
| | | site | SEQ ID NO: 627 |
| 181 | CTTCTGCCGAGTCCTGCAATATGAA | Acceptor | TTCATATTGCAGGACTCGGCAGAAG |
| | | site | SEQ ID NO: 628 |
| 182 | TCTTCTGCCGAGTCCTGCAATATGA | Acceptor | TCATATTGCAGGACTCGGCAGAAGA |
| | | site | SEQ ID NO: 629 |
| 183 | GTCTTCTGCCGAGTCCTGCAATATG | Acceptor | CATATTGCAGGACTCGGCAGAAGAC |
| | | site | SEQ ID NO: 630 |
| 184 | GGTCTTCTGCCGAGTCCTGCAATAT | Acceptor | ATATTGCAGGACTCGGCAGAAGACC |
| | | site | SEQ ID NO: 631 |
| 185 | AGGTCTTCTGCCGAGTCCTGCAATA | Acceptor | TATTGCAGGACTCGGCAGAAGACCT |
| | | site | SEQ ID NO: 632 |
| 186 | AAGGTCTTCTGCCGAGTCCTGCAAT | Acceptor | ATTGCAGGACTCGGCAGAAGACCTT |
| | | site | SEQ ID NO: 633 |
| 187 | GAAGGTCTTCTGCCGAGTCCTGCAA | Acceptor | TTGCAGGACTCGGCAGAAGACCTTC |
| | | site | SEQ ID NO: 634 |
| 188 | CGAAGGTCTTCTGCCGAGTCCTGCA | Acceptor | TGCAGGACTCGGCAGAAGACCTTCG |
| | | site | SEQ ID NO: 635 |
| 189 | TCGAAGGTCTTCTGCCGAGTCCTGC | Acceptor | GCAGGACTCGGCAGAAGACCTTCGA |
| | | site | SEQ ID NO: 636 |
| 190 | CTCGAAGGTCTTCTGCCGAGTCCTG | Acceptor | CAGGACTCGGCAGAAGACCTTCGAG |
| | | site | SEQ ID NO: 637 |
| 191 | TCTCGAAGGTCTTCTGCCGAGTCCT | ESE | AGGACTCGGCAGAAGACCTTCGAGA |
| | | Binding | SEQ ID NO: 638 |
| 192 | CTCTCGAAGGTCTTCTGCCGAGTCC | ESE | GGACTCGGCAGAAGACCTTCGAGAG |
| | | Binding | SEQ ID NO: 639 |
| 193 | TCTCTCGAAGGTCTTCTGCCGAGTC | ESE | GACTCGGCAGAAGACCTTCGAGAGA |
| | | Binding | SEQ ID NO: 640 |
| 194 | TTCTCTCGAAGGTCTTCTGCCGAGT | ESE | ACTCGGCAGAAGACCTTCGAGAGAA |
| | | Binding | SEQ ID NO: 641 |
| 195 | TTTCTCTCGAAGGTCTTCTGCCGAG | ESE | CTCGGCAGAAGACCTTCGAGAGAAA |
| | | Binding | SEQ ID NO: 642 |
| 196 | CTTTCTCTCGAAGGTCTTCTGCCGA | ESE | TCGGCAGAAGACCTTCGAGAGAAAG |
| | | Binding | SEQ ID NO: 643 |
| 197 | CCTTTCTCTCGAAGGTCTTCTGCCG | ESE | CGGCAGAAGACCTTCGAGAGAAAGG |
| | | Binding | SEQ ID NO: 644 |
| 198 | ACCTTTCTCTCGAAGGTCTTCTGCC | ESE | GGCAGAAGACCTTCGAGAGAAAGGT |
| | | Binding | SEQ ID NO: 645 |
| 199 | TACCTTTCTCTCGAAGGTCTTCTGC | ESE | GCAGAAGACCTTCGAGAGAAAGGTA |
| | | Binding | SEQ ID NO: 646 |
| 200 | CTACCTTTCTCTCGAAGGTCTTCTG | ESE | CAGAAGACCTTCGAGAGAAAGGTAG |
| | | Binding | SEQ ID NO: 647 |
| 201 | TCTACCTTTCTCTCGAAGGTCTTCT | ESE | AGAAGACCTTCGAGAGAAAGGTAGA |
| | | Binding | SEQ ID NO: 648 |
| 202 | TTCTACCTTTCTCTCGAAGGTCTTC | ESE | GAAGACCTTCGAGAGAAAGGTAGAA |
| | | Binding | SEQ ID NO: 649 |
| 203 | TTTCTACCTTTCTCTCGAAGGTCTT | ESE | AAGACCTTCGAGAGAAAGGTAGAAA |
| | | Binding | SEQ ID NO: 650 |
| 204 | TTTTCTACCTTTCTCTCGAAGGTCT | ESE | AGACCTTCGAGAGAAAGGTAGAAAA |
| | | Binding | SEQ ID NO: 651 |
| 205 | ATTTTCTACCTTTCTCTCGAAGGTC | ESE | GACCTTCGAGAGAAAGGTAGAAAAT |
| | | Binding | SEQ ID NO: 652 |
| 206 | TATTTTCTACCTTTCTCTCGAAGGT | ESE | ACCTTCGAGAGAAAGGTAGAAAATA |
| | | Binding | SEQ ID NO: 653 |
| 207 | TTATTTTCTACCTTTCTCTCGAAGG | ESE | CCTTCGAGAGAAAGGTAGAAAATAA |
| | | Binding | SEQ ID NO: 654 |
| 208 | CTTATTTTCTACCTTTCTCTCGAAG | ESE | CTTCGAGAGAAAGGTAGAAAATAAG |
| | | Binding | SEQ ID NO: 655 |
| 209 | TCTTATTTTCTACCTTTCTCTCGAA | ESE | TTCGAGAGAAAGGTAGAAAATAAGA |
| | | Binding | SEQ ID NO: 656 |
| 210 | TTCTTATTTTCTACCTTTCTCTCGA | ESE | TCGAGAGAAAGGTAGAAAATAAGAA |
| | | Binding | SEQ ID NO: 657 |
| 211 | ATTCTTATTTTCTACCTTTCTCTCG | ESE | CGAGAGAAAGGTAGAAAATAAGAAT |
| | | Binding | SEQ ID NO: 658 |
| 212 | AATTCTTATTTTCTACCTTTCTCTC | ESE | GAGAGAAAGGTAGAAAATAAGAATT |
| | | Binding | SEQ ID NO: 659 |
| 213 | AAATTCTTATTTTCTACCTTTCTCT | ESE | AGAGAAAGGTAGAAAATAAGAATTT |
| | | Binding | SEQ ID NO: 660 |
| 214 | CAAATTCTTATTTTCTACCTTTCTC | ESE | GAGAAAGGTAGAAAATAAGAATTTG |
| | | Binding | SEQ ID NO: 661 |
| 215 | CCAAATTCTTATTTTCTACCTTTCT | ESE | AGAAAGGTAGAAAATAAGAATTTGG |
| | | Binding | SEQ ID NO: 662 |
| 216 | GCCAAATTCTTATTTTCTACCTTTC | ESE | GAAAGGTAGAAAATAAGAATTTGGC |
| | | Binding | SEQ ID NO: 663 |
| 217 | AGCCAAATTCTTATTTTCTACCTTT | ESE | AAAGGTAGAAAATAAGAATTTGGCT |
| | | Binding | SEQ ID NO: 664 |
| 218 | GAGCCAAATTCTTATTTTCTACCTT | ESE | AAGGTAGAAAATAAGAATTTGGCTC |
| | | Binding | SEQ ID NO: 665 |
| 219 | AGAGCCAAATTCTTATTTTCTACCT | ESE | AGGTAGAAAATAAGAATTTGGCTCT |
| | | Binding | SEQ ID NO: 666 |
| 220 | GAGAGCCAAATTCTTATTTTCTACC | ESE | GGTAGAAAATAAGAATTTGGCTCTC |
| | | Binding | SEQ ID NO: 667 |
| 221 | AGAGAGCCAAATTCTTATTTTCTAC | ESE | GTAGAAAATAAGAATTTGGCTCTCT |
| | | Binding | SEQ ID NO: 668 |
| 222 | CAGAGAGCCAAATTCTTATTTTCTA | | TAGAAAATAAGAATTTGGCTCTCTG |
| | | | SEQ ID NO: 669 |
| 223 | ACAGAGAGCCAAATTCTTATTTTCT | | AGAAAATAAGAATTTGGCTCTCTGT |
| | | | SEQ ID NO: 670 |
| 224 | CACAGAGAGCCAAATTCTTATTTTC | | GAAAATAAGAATTTGGCTCTCTGTG |
| | | | SEQ ID NO: 671 |
| 225 | ACACAGAGAGCCAAATTCTTATTTT | | AAAATAAGAATTTGGCTCTCTGTGT |
| | | | SEQ ID NO: 672 |
| 226 | CACACAGAGAGCCAAATTCTTATTT | Overlaps | AAATAAGAATTTGGCTCTCTGTGTG |
| | | TDP-43 | SEQ ID NO: 673 |
| | | site 1 | |
| 227 | TCACACAGAGAGCCAAATTCTTATT | Overlaps | AATAAGAATTTGGCTCTCTGTGTGA |
| | | TDP-43 | SEQ ID NO: 674 |
| | | site 1 | |
| 228 | CTCACACAGAGAGCCAAATTCTTAT | Overlaps | ATAAGAATTTGGCTCTCTGTGTGAG |
| | | TDP-43 | SEQ ID NO: 675 |
| | | site 1 | |
| 229 | GCTCACACAGAGAGCCAAATTCTTA | Overlaps | TAAGAATTTGGCTCTCTGTGTGAGC |
| | | TDP-43 | SEQ ID NO: 676 |
| | | site 1 | |
| 230 | TGCTCACACAGAGAGCCAAATTCTT | Overlaps | AAGAATTTGGCTCTCTGTGTGAGCA |
| | | TDP-43 | SEQ ID NO: 677 |
| | | site 1 | |
| 231 | ATGCTCACACAGAGAGCCAAATTCT | Overlaps | AGAATTTGGCTCTCTGTGTGAGCAT |
| | | TDP-43 | SEQ ID NO: 678 |
| | | site 1 | |
| 232 | CATGCTCACACAGAGAGCCAAATTC | Overlaps | GAATTTGGCTCTCTGTGTGAGCATG |
| | | TDP-43 | SEQ ID NO: 679 |
| | | site 1 | |
| 233 | ACATGCTCACACAGAGAGCCAAATT | Overlaps | AATTTGGCTCTCTGTGTGAGCATGT |
| | | TDP-43 | SEQ ID NO: 680 |
| | | site 1 | |
| 234 | CACATGCTCACACAGAGAGCCAAAT | Overlaps | ATTTGGCTCTCTGTGTGAGCATGTG |
| | | TDP-43 | SEQ ID NO: 681 |
| | | site 1 | |
| 235 | ACACATGCTCACACAGAGAGCCAAA | Overlaps | TTTGGCTCTCTGTGTGAGCATGTGT |
| | | TDP-43 | SEQ ID NO: 682 |
| | | site 1 | |
| 236 | CACACATGCTCACACAGAGAGCCAA | Overlaps | TTGGCTCTCTGTGTGAGCATGTGTG |
| | | TDP-43 | SEQ ID NO: 683 |
| | | site 1 & | |
| | | 2 | |
| 237 | GCACACATGCTCACACAGAGAGCCA | Overlaps | TGGCTCTCTGTGTGAGCATGTGTGC |
| | | TDP-43 | SEQ ID NO: 684 |
| | | site 1 & | |
| | | 2 | |
| 238 | CGCACACATGCTCACACAGAGAGCC | Overlaps | GGCTCTCTGTGTGAGCATGTGTGCG |
| | | TDP-43 | SEQ ID NO: 685 |
| | | site 1 & | |
| | | 2 | |
| 239 | ACGCACACATGCTCACACAGAGAGC | Overlaps | GCTCTCTGTGTGAGCATGTGTGCGT |
| | | TDP-43 | SEQ ID NO: 686 |
| | | site 1 & | |
| | | 2 | |
| 240 | CACGCACACATGCTCACACAGAGAG | Overlaps | CTCTCTGTGTGAGCATGTGTGCGTG |
| | | TDP-43 | SEQ ID NO: 687 |
| | | site 1 & | |
| | | 2 | |
| 241 | ACACGCACACATGCTCACACAGAGA | Overlaps | TCTCTGTGTGAGCATGTGTGCGTGT |
| | | TDP-43 | SEQ ID NO: 688 |
| | | site 1 & | |
| | | 2 | |
| 242 | CACACGCACACATGCTCACACAGAG | Overlaps | CTCTGTGTGAGCATGTGTGCGTGTG |
| | | TDP-43 | SEQ ID NO: 689 |
| | | site 1 & | |
| | | 2 | |
| 243 | ACACACGCACACATGCTCACACAGA | Overlaps | TCTGTGTGAGCATGTGTGCGTGTGT |
| | | TDP-43 | SEQ ID NO: 690 |
| | | site 1 & | |
| | | 2 | |
| 244 | CACACACGCACACATGCTCACACAG | Overlaps | CTGTGTGAGCATGTGTGCGTGTGTG |
| | | TDP-43 | SEQ ID NO: 691 |
| | | site 1 & | |
| | | 2 & 3 | |
| 245 | GCACACACGCACACATGCTCACACA | Overlaps | TGTGTGAGCATGTGTGCGTGTGTGC |
| | | TDP-43 | SEQ ID NO: 692 |
| | | site 1 & | |
| | | 2 & 3 | |
| 246 | CGCACACACGCACACATGCTCACAC | Overlaps | GTGTGAGCATGTGTGCGTGTGTGCG |
| | | TDP-43 | SEQ ID NO: 693 |
| | | site 2 & | |
| | | 3 | |
| 247 | TCGCACACACGCACACATGCTCACA | Overlaps | TGTGAGCATGTGTGCGTGTGTGCGA |
| | | TDP-43 | SEQ ID NO: 694 |
| | | site 2 & | |
| | | 3 | |
| 248 | CTCGCACACACGCACACATGCTCAC | Overlaps | GTGAGCATGTGTGCGTGTGTGCGAG |
| | | TDP-43 | SEQ ID NO: 695 |
| | | site 2 & | |
| | | 3 | |
| 249 | TCTCGCACACACGCACACATGCTCA | Overlaps | TGAGCATGTGTGCGTGTGTGCGAGA |
| | | TDP-43 | SEQ ID NO: 696 |
| | | site 2 & | |
| | | 3 | |
| 250 | CTCTCGCACACACGCACACATGCTC | Overlaps | GAGCATGTGTGCGTGTGTGCGAGAG |
| | | TDP-43 | SEQ ID NO: 697 |
| | | site 2 & | |
| | | 3 | |
| 251 | TCTCTCGCACACACGCACACATGCT | Overlaps | AGCATGTGTGCGTGTGTGCGAGAGA |
| | | TDP-43 | SEQ ID NO: 698 |
| | | site 2 & | |
| | | 3 | |
| 252 | CTCTCTCGCACACACGCACACATGC | Overlaps | GCATGTGTGCGTGTGTGCGAGAGAG |
| | | TDP-43 | SEQ ID NO: 699 |
| | | site 2 & | |
| | | 3 | |
| 253 | TCTCTCTCGCACACACGCACACATG | Overlaps | CATGTGTGCGTGTGTGCGAGAGAGA |
| | | TDP-43 | SEQ ID NO: 700 |
| | | site 2 & | |
| | | 3 | |
| 254 | CTCTCTCTCGCACACACGCACACAT | Overlaps | ATGTGTGCGTGTGTGCGAGAGAGAG |
| | | TDP-43 | SEQ ID NO: 701 |
| | | site 2 & | |
| | | 3 | |
| 255 | TCTCTCTCTCGCACACACGCACACA | Overlaps | TGTGTGCGTGTGTGCGAGAGAGAGA |
| | | TDP-43 | SEQ ID NO: 702 |
| | | site 2 & | |
| | | 3 | |
| 256 | CTCTCTCTCTCGCACACACGCACAC | Overlaps | GTGTGCGTGTGTGCGAGAGAGAGAG |
| | | TDP-43 | SEQ ID NO: 703 |
| | | site 3 | |
| 257 | TCTCTCTCTCTCGCACACACGCACA | Overlaps | TGTGCGTGTGTGCGAGAGAGAGAGA |
| | | TDP-43 | SEQ ID NO: 704 |
| | | site 3 | |
| 258 | GTCTCTCTCTCTCGCACACACGCAC | Overlaps | GTGCGTGTGTGCGAGAGAGAGAGAC |
| | | TDP-43 | SEQ ID NO: 705 |
| | | site 3 | |
| 259 | TGTCTCTCTCTCTCGCACACACGCA | Overlaps | TGCGTGTGTGCGAGAGAGAGAGACA |
| | | TDP-43 | SEQ ID NO: 706 |
| | | site 3 | |
| 260 | CTGTCTCTCTCTCTCGCACACACGC | Overlaps | GCGTGTGTGCGAGAGAGAGAGACAG |
| | | TDP-43 | SEQ ID NO: 707 |
| | | site 3 | |
| 261 | TCTGTCTCTCTCTCTCGCACACACG | Overlaps | CGTGTGTGCGAGAGAGAGAGACAGA |
| | | TDP-43 | SEQ ID NO: 708 |
| | | site 3 | |
| 262 | GTCTGTCTCTCTCTCTCGCACACAC | Overlaps | GTGTGTGCGAGAGAGAGAGACAGAC |
| | | TDP-43 | SEQ ID NO: 709 |
| | | site 3 | |
| 263 | TGTCTGTCTCTCTCTCTCGCACACA | Overlaps | TGTGTGCGAGAGAGAGAGACAGACA |
| | | TDP-43 | SEQ ID NO: 710 |
| | | site 3 | |
| 264 | CTGTCTGTCTCTCTCTCTCGCACAC | | GTGTGCGAGAGAGAGAGACAGACAG |
| | | | SEQ ID NO: 711 |
| 265 | GCTGTCTGTCTCTCTCTCTCGCACA | | TGTGCGAGAGAGAGAGACAGACAGC |
| | | | SEQ ID NO: 712 |
| 266 | GGCTGTCTGTCTCTCTCTCTCGCAC | | GTGCGAGAGAGAGAGACAGACAGCC |
| | | | SEQ ID NO: 713 |
| 267 | AGGCTGTCTGTCTCTCTCTCTCGCA | | TGCGAGAGAGAGAGACAGACAGCCT |
| | | | SEQ ID NO: 714 |
| 268 | CAGGCTGTCTGTCTCTCTCTCTCGC | | GCGAGAGAGAGAGACAGACAGCCTG |
| | | | SEQ ID NO: 715 |
| 269 | GCAGGCTGTCTGTCTCTCTCTCTCG | | CGAGAGAGAGAGACAGACAGCCTGC |
| | | | SEQ ID NO: 716 |
| 270 | GGCAGGCTGTCTGTCTCTCTCTCTC | | GAGAGAGAGAGACAGACAGCCTGCC |
| | | | SEQ ID NO: 717 |
| 271 | AGGCAGGCTGTCTGTCTCTCTCTCT | | AGAGAGAGAGACAGACAGCCTGCCT |
| | | | SEQ ID NO: 718 |
| 272 | TAGGCAGGCTGTCTGTCTCTCTCTC | | GAGAGAGAGACAGACAGCCTGCCTA |
| | | | SEQ ID NO: 719 |
| 273 | TTAGGCAGGCTGTCTGTCTCTCTCT | | AGAGAGAGACAGACAGCCTGCCTAA |
| | | | SEQ ID NO: 720 |
| 274 | CTTAGGCAGGCTGTCTGTCTCTCTC | | GAGAGAGACAGACAGCCTGCCTAAG |
| | | | SEQ ID NO: 721 |
| 275 | TCTTAGGCAGGCTGTCTGTCTCTCT | | AGAGAGACAGACAGCCTGCCTAAGA |
| | | | SEQ ID NO: 722 |
| 276 | TTCTTAGGCAGGCTGTCTGTCTCTC | | GAGAGACAGACAGCCTGCCTAAGAA |
| | | | SEQ ID NO: 723 |
| 277 | CTTCTTAGGCAGGCTGTCTGTCTCT | | AGAGACAGACAGCCTGCCTAAGAAG |
| | | | SEQ ID NO: 724 |
| 278 | TCTTCTTAGGCAGGCTGTCTGTCTC | | GAGACAGACAGCCTGCCTAAGAAGA |
| | | | SEQ ID NO: 725 |
| 279 | TTCTTCTTAGGCAGGCTGTCTGTCT | | AGACAGACAGCCTGCCTAAGAAGAA |
| | | | SEQ ID NO: 726 |
| 280 | TTTCTTCTTAGGCAGGCTGTCTGTC | | GACAGACAGCCTGCCTAAGAAGAAA |
| | | | SEQ ID NO: 727 |
| 281 | ATTTCTTCTTAGGCAGGCTGTCTGT | | ACAGACAGCCTGCCTAAGAAGAAAT |
| | | | SEQ ID NO: 728 |
| 282 | CATTTCTTCTTAGGCAGGCTGTCTG | | CAGACAGCCTGCCTAAGAAGAAATG |
| | | | SEQ ID NO: 729 |
| 283 | TCATTTCTTCTTAGGCAGGCTGTCT | | AGACAGCCTGCCTAAGAAGAAATGA |
| | | | SEQ ID NO: 730 |
| 284 | TTCATTTCTTCTTAGGCAGGCTGTC | | GACAGCCTGCCTAAGAAGAAATGAA |
| | | | SEQ ID NO: 731 |
| 285 | ATTCATTTCTTCTTAGGCAGGCTGT | | ACAGCCTGCCTAAGAAGAAATGAAT |
| | | | SEQ ID NO: 732 |
| 286 | CATTCATTTCTTCTTAGGCAGGCTG | | CAGCCTGCCTAAGAAGAAATGAATG |
| | | | SEQ ID NO: 733 |
| 287 | ACATTCATTTCTTCTTAGGCAGGCT | | AGCCTGCCTAAGAAGAAATGAATGT |
| | | | SEQ ID NO: 734 |
| 288 | CACATTCATTTCTTCTTAGGCAGGC | | GCCTGCCTAAGAAGAAATGAATGTG |
| | | | SEQ ID NO: 735 |
| 289 | TCACATTCATTTCTTCTTAGGCAGG | | CCTGCCTAAGAAGAAATGAATGTGA |
| | | | SEQ ID NO: 736 |
| 290 | TTCACATTCATTTCTTCTTAGGCAG | | CTGCCTAAGAAGAAATGAATGTGAA |
| | | | SEQ ID NO: 737 |
| 291 | ATTCACATTCATTTCTTCTTAGGCA | | TGCCTAAGAAGAAATGAATGTGAAT |
| | | | SEQ ID NO: 738 |
| 292 | CATTCACATTCATTTCTTCTTAGGC | | GCCTAAGAAGAAATGAATGTGAATG |
| | | | SEQ ID NO: 739 |
| 293 | GCATTCACATTCATTTCTTCTTAGG | | CCTAAGAAGAAATGAATGTGAATGC |
| | | | SEQ ID NO: 740 |
| 294 | CGCATTCACATTCATTTCTTCTTAG | | CTAAGAAGAAATGAATGTGAATGCG |
| | | | SEQ ID NO: 741 |
| 295 | CCGCATTCACATTCATTTCTTCTTA | | TAAGAAGAAATGAATGTGAATGCGG |
| | | | SEQ ID NO: 742 |
| 296 | GCCGCATTCACATTCATTTCTTCTT | | AAGAAGAAATGAATGTGAATGCGGC |
| | | | SEQ ID NO: 743 |
| 297 | AGCCGCATTCACATTCATTTCTTCT | | AGAAGAAATGAATGTGAATGCGGCT |
| | | | SEQ ID NO: 744 |
| 298 | AAGCCGCATTCACATTCATTTCTTC | | GAAGAAATGAATGTGAATGCGGCTT |
| | | | SEQ ID NO: 745 |
| 299 | CAAGCCGCATTCACATTCATTTCTT | | AAGAAATGAATGTGAATGCGGCTTG |
| | | | SEQ ID NO: 746 |
| 300 | ACAAGCCGCATTCACATTCATTTCT | | AGAAATGAATGTGAATGCGGCTTGT |
| | | | SEQ ID NO: 747 |
| 301 | CACAAGCCGCATTCACATTCATTTC | | GAAATGAATGTGAATGCGGCTTGTG |
| | | | SEQ ID NO: 748 |
| 302 | CCACAAGCCGCATTCACATTCATTT | | AAATGAATGTGAATGCGGCTTGTGG |
| | | | SEQ ID NO: 749 |
| 303 | GCCACAAGCCGCATTCACATTCATT | | AATGAATGTGAATGCGGCTTGTGGC |
| | | | SEQ ID NO: 750 |
| 304 | TGCCACAAGCCGCATTCACATTCAT | | ATGAATGTGAATGCGGCTTGTGGCA |
| | | | SEQ ID NO: 751 |
| 305 | GTGCCACAAGCCGCATTCACATTCA | | TGAATGTGAATGCGGCTTGTGGCAC |
| | | | SEQ ID NO: 752 |
| 306 | TGTGCCACAAGCCGCATTCACATTC | | GAATGTGAATGCGGCTTGTGGCACA |
| | | | SEQ ID NO: 753 |
| 307 | CTGTGCCACAAGCCGCATTCACATT | | AATGTGAATGCGGCTTGTGGCACAG |
| | | | SEQ ID NO: 754 |
| 308 | ACTGTGCCACAAGCCGCATTCACAT | | ATGTGAATGCGGCTTGTGGCACAGT |
| | | | SEQ ID NO: 755 |
| 309 | AACTGTGCCACAAGCCGCATTCACA | | TGTGAATGCGGCTTGTGGCACAGTT |
| | | | SEQ ID NO: 756 |
| 310 | CAACTGTGCCACAAGCCGCATTCAC | | GTGAATGCGGCTTGTGGCACAGTTG |
| | | | SEQ ID NO: 757 |
| 311 | TCAACTGTGCCACAAGCCGCATTCA | | TGAATGCGGCTTGTGGCACAGTTGA |
| | | | SEQ ID NO: 758 |
| 312 | GTCAACTGTGCCACAAGCCGCATTC | | GAATGCGGCTTGTGGCACAGTTGAC |
| | | | SEQ ID NO: 759 |
| 313 | TGTCAACTGTGCCACAAGCCGCATT | | AATGCGGCTTGTGGCACAGTTGACA |
| | | | SEQ ID NO: 760 |
| 314 | TTGTCAACTGTGCCACAAGCCGCAT | | ATGCGGCTTGTGGCACAGTTGACAA |
| | | | SEQ ID NO: 761 |
| 315 | CTTGTCAACTGTGCCACAAGCCGCA | | TGCGGCTTGTGGCACAGTTGACAAG |
| | | | SEQ ID NO: 762 |
| 316 | CCTTGTCAACTGTGCCACAAGCCGC | | GCGGCTTGTGGCACAGTTGACAAGG |
| | | | SEQ ID NO: 763 |
| 317 | TCCTTGTCAACTGTGCCACAAGCCG | | CGGCTTGTGGCACAGTTGACAAGGA |
| | | | SEQ ID NO: 764 |
| 318 | ATCCTTGTCAACTGTGCCACAAGCC | | GGCTTGTGGCACAGTTGACAAGGAT |
| | | | SEQ ID NO: 765 |
| 319 | CATCCTTGTCAACTGTGCCACAAGC | | GCTTGTGGCACAGTTGACAAGGATG |
| | | | SEQ ID NO: 766 |
| 320 | TCATCCTTGTCAACTGTGCCACAAG | | CTTGTGGCACAGTTGACAAGGATGA |
| | | | SEQ ID NO: 767 |
| 321 | ATCATCCTTGTCAACTGTGCCACAA | | TTGTGGCACAGTTGACAAGGATGAT |
| | | | SEQ ID NO: 768 |
| 322 | TATCATCCTTGTCAACTGTGCCACA | | TGTGGCACAGTTGACAAGGATGATA |
| | | | SEQ ID NO: 769 |
| 323 | TTATCATCCTTGTCAACTGTGCCAC | | GTGGCACAGTTGACAAGGATGATAA |
| | | | SEQ ID NO: 770 |
| 324 | TTTATCATCCTTGTCAACTGTGCCA | | TGGCACAGTTGACAAGGATGATAAA |
| | | | SEQ ID NO: 771 |
| 325 | ATTTATCATCCTTGTCAACTGTGCC | | GGCACAGTTGACAAGGATGATAAAT |
| | | | SEQ ID NO: 772 |
| 326 | GATTTATCATCCTTGTCAACTGTGC | | GCACAGTTGACAAGGATGATAAATC |
| | | | SEQ ID NO: 773 |
| 327 | TGATTTATCATCCTTGTCAACTGTG | | CACAGTTGACAAGGATGATAAATCA |
| | | | SEQ ID NO: 774 |
| 328 | TTGATTTATCATCCTTGTCAACTGT | | ACAGTTGACAAGGATGATAAATCAA |
| | | | SEQ ID NO: 775 |
| 329 | ATTGATTTATCATCCTTGTCAACTG | | CAGTTGACAAGGATGATAAATCAAT |
| | | | SEQ ID NO: 776 |
| 330 | TATTGATTTATCATCCTTGTCAACT | | AGTTGACAAGGATGATAAATCAATA |
| | | | SEQ ID NO: 777 |
| 331 | TTATTGATTTATCATCCTTGTCAAC | | GTTGACAAGGATGATAAATCAATAA |
| | | | SEQ ID NO: 778 |
| 332 | ATTATTGATTTATCATCCTTGTCAA | | TTGACAAGGATGATAAATCAATAAT |
| | | | SEQ ID NO: 779 |
| 333 | CATTATTGATTTATCATCCTTGTCA | | TGACAAGGATGATAAATCAATAATG |
| | | | SEQ ID NO: 780 |
| 334 | GCATTATTGATTTATCATCCTTGTC | | GACAAGGATGATAAATCAATAATGC |
| | | | SEQ ID NO: 781 |
| 335 | TGCATTATTGATTTATCATCCTTGT | | ACAAGGATGATAAATCAATAATGCA |
| | | | SEQ ID NO: 782 |
| 336 | TTGCATTATTGATTTATCATCCTTG | | CAAGGATGATAAATCAATAATGCAA |
| | | | SEQ ID NO: 783 |
| 337 | CTTGCATTATTGATTTATCATCCTT | | AAGGATGATAAATCAATAATGCAAG |
| | | | SEQ ID NO: 784 |
| 338 | GCTTGCATTATTGATTTATCATCCT | | AGGATGATAAATCAATAATGCAAGC |
| | | | SEQ ID NO: 785 |
| 339 | AGCTTGCATTATTGATTTATCATCC | | GGATGATAAATCAATAATGCAAGCT |
| | | | SEQ ID NO: 786 |
| 340 | AAGCTTGCATTATTGATTTATCATC | | GATGATAAATCAATAATGCAAGCTT |
| | | | SEQ ID NO: 787 |
| 341 | TAAGCTTGCATTATTGATTTATCAT | | ATGATAAATCAATAATGCAAGCTTA |
| | | | SEQ ID NO: 788 |
| 342 | GTAAGCTTGCATTATTGATTTATCA | | TGATAAATCAATAATGCAAGCTTAC |
| | | | SEQ ID NO: 789 |
| 343 | AGTAAGCTTGCATTATTGATTTATC | | GATAAATCAATAATGCAAGCTTACT |
| | | | SEQ ID NO: 790 |
| 344 | TAGTAAGCTTGCATTATTGATTTAT | | ATAAATCAATAATGCAAGCTTACTA |
| | | | SEQ ID NO: 791 |
| 345 | ATAGTAAGCTTGCATTATTGATTTA | | TAAATCAATAATGCAAGCTTACTAT |
| | | | SEQ ID NO: 792 |
| 346 | GATAGTAAGCTTGCATTATTGATTT | | AAATCAATAATGCAAGCTTACTATC |
| | | | SEQ ID NO: 793 |
| 347 | TGATAGTAAGCTTGCATTATTGATT | | AATCAATAATGCAAGCTTACTATCA |
| | | | SEQ ID NO: 794 |
| 348 | ATGATAGTAAGCTTGCATTATTGAT | | ATCAATAATGCAAGCTTACTATCAT |
| | | | SEQ ID NO: 795 |
| 349 | AATGATAGTAAGCTTGCATTATTGA | | TCAATAATGCAAGCTTACTATCATT |
| | | | SEQ ID NO: 796 |
| 350 | AAATGATAGTAAGCTTGCATTATTG | | CAATAATGCAAGCTTACTATCATTT |
| | | | SEQ ID NO: 797 |
| 351 | TAAATGATAGTAAGCTTGCATTATT | | AATAATGCAAGCTTACTATCATTTA |
| | | | SEQ ID NO: 798 |
| 352 | ATAAATGATAGTAAGCTTGCATTAT | | ATAATGCAAGCTTACTATCATTTAT |
| | | | SEQ ID NO: 799 |
| 353 | CATAAATGATAGTAAGCTTGCATTA | | TAATGCAAGCTTACTATCATTTATG |
| | | | SEQ ID NO: 800 |
| 354 | TCATAAATGATAGTAAGCTTGCATT | | AATGCAAGCTTACTATCATTTATGA |
| | | | SEQ ID NO: 801 |
| 355 | TTCATAAATGATAGTAAGCTTGCAT | | ATGCAAGCTTACTATCATTTATGAA |
| | | | SEQ ID NO: 802 |
| 356 | ATTCATAAATGATAGTAAGCTTGCA | | TGCAAGCTTACTATCATTTATGAAT |
| | | | SEQ ID NO: 803 |
| 357 | TATTCATAAATGATAGTAAGCTTGC | | GCAAGCTTACTATCATTTATGAATA |
| | | | SEQ ID NO: 804 |
| 358 | CTATTCATAAATGATAGTAAGCTTG | | CAAGCTTACTATCATTTATGAATAG |
| | | | SEQ ID NO: 805 |
| 359 | GCTATTCATAAATGATAGTAAGCTT | | AAGCTTACTATCATTTATGAATAGC |
| | | | SEQ ID NO: 806 |
| 360 | TGCTATTCATAAATGATAGTAAGCT | | AGCTTACTATCATTTATGAATAGCA |
| | | | SEQ ID NO: 807 |
| 361 | TTGCTATTCATAAATGATAGTAAGC | | GCTTACTATCATTTATGAATAGCAA |
| | | | SEQ ID NO: 808 |
| 362 | ATTGCTATTCATAAATGATAGTAAG | | CTTACTATCATTTATGAATAGCAAT |
| | | | SEQ ID NO: 809 |
| 363 | TATTGCTATTCATAAATGATAGTAA | | TTACTATCATTTATGAATAGCAATA |
| | | | SEQ ID NO: 810 |
| 364 | GTATTGCTATTCATAAATGATAGTA | | TACTATCATTTATGAATAGCAATAC |
| | | | SEQ ID NO: 811 |
| 365 | AGTATTGCTATTCATAAATGATAGT | | ACTATCATTTATGAATAGCAATACT |
| | | | SEQ ID NO: 812 |
| 366 | CAGTATTGCTATTCATAAATGATAG | | CTATCATTTATGAATAGCAATACTG |
| | | | SEQ ID NO: 813 |
| 367 | TCAGTATTGCTATTCATAAATGATA | | TATCATTTATGAATAGCAATACTGA |
| | | | SEQ ID NO: 814 |
| 368 | TTCAGTATTGCTATTCATAAATGAT | | ATCATTTATGAATAGCAATACTGAA |
| | | | SEQ ID NO: 815 |
| 369 | CTTCAGTATTGCTATTCATAAATGA | | TCATTTATGAATAGCAATACTGAAG |
| | | | SEQ ID NO: 816 |
| 370 | TCTTCAGTATTGCTATTCATAAATG | | CATTTATGAATAGCAATACTGAAGA |
| | | | SEQ ID NO: 817 |
| 371 | TTCTTCAGTATTGCTATTCATAAAT | | ATTTATGAATAGCAATACTGAAGAA |
| | | | SEQ ID NO: 818 |
| 372 | TTTCTTCAGTATTGCTATTCATAAA | | TTTATGAATAGCAATACTGAAGAAA |
| | | | SEQ ID NO: 819 |
| 373 | ATTTCTTCAGTATTGCTATTCATAA | | TTATGAATAGCAATACTGAAGAAAT |
| | | | SEQ ID NO: 820 |
| 374 | AATTTCTTCAGTATTGCTATTCATA | | TATGAATAGCAATACTGAAGAAATT |
| | | | SEQ ID NO: 821 |
| 375 | TAATTTCTTCAGTATTGCTATTCAT | | ATGAATAGCAATACTGAAGAAATTA |
| | | | SEQ ID NO: 822 |
| 376 | TTAATTTCTTCAGTATTGCTATTCA | | TGAATAGCAATACTGAAGAAATTAA |
| | | | SEQ ID NO: 823 |
| 377 | TTTAATTTCTTCAGTATTGCTATTC | polyA | GAATAGCAATACTGAAGAAATTAAA |
| | | signal | SEQ ID NO: 824 |
| 378 | TTTTAATTTCTTCAGTATTGCTATT | polyA | AATAGCAATACTGAAGAAATTAAAA |
| | | signal | SEQ ID NO: 825 |
| 379 | GTTTTAATTTCTTCAGTATTGCTAT | polyA | ATAGCAATACTGAAGAAATTAAAAC |
| | | signal | SEQ ID NO: 826 |
| 380 | TGTTTTAATTTCTTCAGTATTGCTA | polyA | TAGCAATACTGAAGAAATTAAAACA |
| | | signal | SEQ ID NO: 827 |
| 381 | TTGTTTTAATTTCTTCAGTATTGCT | polyA | AGCAATACTGAAGAAATTAAAACAA |
| | | signal | SEQ ID NO: 828 |
| 382 | TTTGTTTTAATTTCTTCAGTATTGC | polyA | GCAATACTGAAGAAATTAAAACAAA |
| | | signal | SEQ ID NO: 829 |
| 383 | TTTTGTTTTAATTTCTTCAGTATTG | polyA | CAATACTGAAGAAATTAAAACAAAA |
| | | signal | SEQ ID NO: 830 |
| 384 | CTTTTGTTTTAATTTCTTCAGTATT | polyA | AATACTGAAGAAATTAAAACAAAAG |
| | | signal | SEQ ID NO: 831 |
| 385 | TCTTTTGTTTTAATTTCTTCAGTAT | polyA | ATACTGAAGAAATTAAAACAAAAGA |
| | | signal | SEQ ID NO: 832 |
| 386 | ATCTTTTGTTTTAATTTCTTCAGTA | polyA | TACTGAAGAAATTAAAACAAAAGAT |
| | | signal | SEQ ID NO: 833 |
| 387 | AATCTTTTGTTTTAATTTCTTCAGT | polyA | ACTGAAGAAATTAAAACAAAAGATT |
| | | signal | SEQ ID NO: 834 |
| 388 | CAATCTTTTGTTTTAATTTCTTCAG | polyA | CTGAAGAAATTAAAACAAAAGATTG |
| | | signal | SEQ ID NO: 835 |
| 389 | GCAATCTTTTGTTTTAATTTCTTCA | polyA | TGAAGAAATTAAAACAAAAGATTGC |
| | | signal | SEQ ID NO: 836 |
| 390 | AGCAATCTTTTGTTTTAATTTCTTC | polyA | GAAGAAATTAAAACAAAAGATTGCT |
| | | signal | SEQ ID NO: 837 |
| 391 | CAGCAATCTTTTGTTTTAATTTCTT | polyA | AAGAAATTAAAACAAAAGATTGCTG |
| | | signal | SEQ ID NO: 838 |
| 392 | ACAGCAATCTTTTGTTTTAATTTCT | polyA | AGAAATTAAAACAAAAGATTGCTGT |
| | | signal | SEQ ID NO: 839 |
| 393 | GACAGCAATCTTTTGTTTTAATTTC | polyA | GAAATTAAAACAAAAGATTGCTGTC |
| | | signal | SEQ ID NO: 840 |
| 394 | AGACAGCAATCTTTTGTTTTAATTT | polyA | AAATTAAAACAAAAGATTGCTGTCT |
| | | signal | SEQ ID NO: 841 |
| 395 | GAGACAGCAATCTTTTGTTTTAATT | polyA | AATTAAAACAAAAGATTGCTGTCTC |
| | | signal | SEQ ID NO: 842 |
| | | and site | |
| 396 | TGAGACAGCAATCTTTTGTTTTAAT | polyA | ATTAAAACAAAAGATTGCTGTCTCA |
| | | signal | SEQ ID NO: 843 |
| | | and site | |
| 397 | TTGAGACAGCAATCTTTTGTTTTAA | polyA | TTAAAACAAAAGATTGCTGTCTCAA |
| | | site | SEQ ID NO: 844 |
| 398 | ATTGAGACAGCAATCTTTTGTTTTA | polyA | TAAAACAAAAGATTGCTGTCTCAAT |
| | | site | SEQ ID NO: 845 |
| 399 | TATTGAGACAGCAATCTTTTGTTTT | polyA | AAAACAAAAGATTGCTGTCTCAATA |
| | | site | SEQ ID NO: 846 |
| 400 | ATATTGAGACAGCAATCTTTTGTTT | polyA | AAACAAAAGATTGCTGTCTCAATAT |
| | | site | SEQ ID NO: 847 |
| 401 | TATATTGAGACAGCAATCTTTTGTT | polyA | AACAAAAGATTGCTGTCTCAATATA |
| | | site | SEQ ID NO: 848 |
| 402 | ATATATTGAGACAGCAATCTTTTGT | polyA | ACAAAAGATTGCTGTCTCAATATAT |
| | | site | SEQ ID NO: 849 |
| 403 | GATATATTGAGACAGCAATCTTTTG | polyA | CAAAAGATTGCTGTCTCAATATATC |
| | | site | SEQ ID NO: 850 |
| 404 | AGATATATTGAGACAGCAATCTTTT | polyA | AAAAGATTGCTGTCTCAATATATCT |
| | | site | SEQ ID NO: 851 |
| 405 | AAGATATATTGAGACAGCAATCTTT | polyA | AAAGATTGCTGTCTCAATATATCTT |
| | | site | SEQ ID NO: 852 |
| 406 | TAAGATATATTGAGACAGCAATCTT | polyA | AAGATTGCTGTCTCAATATATCTTA |
| | | site | SEQ ID NO: 853 |
| 407 | ATAAGATATATTGAGACAGCAATCT | polyA | AGATTGCTGTCTCAATATATCTTAT |
| | | site | SEQ ID NO: 854 |
| 408 | TATAAGATATATTGAGACAGCAATC | polyA | GATTGCTGTCTCAATATATCTTATA |
| | | site | SEQ ID NO: 855 |
| 409 | ATATAAGATATATTGAGACAGCAAT | polyA | ATTGCTGTCTCAATATATCTTATAT |
| | | site | SEQ ID NO: 856 |
| 410 | AATATAAGATATATTGAGACAGCAA | polyA | TTGCTGTCTCAATATATCTTATATT |
| | | site | SEQ ID NO: 857 |
| 411 | AAATATAAGATATATTGAGACAGCA | polyA | TGCTGTCTCAATATATCTTATATTT |
| | | site | SEQ ID NO: 858 |
| 412 | TAAATATAAGATATATTGAGACAGC | polyA | GCTGTCTCAATATATCTTATATTTA |
| | | site | SEQ ID NO: 859 |
| 413 | ATAAATATAAGATATATTGAGACAG | | CTGTCTCAATATATCTTATATTTAT |
| | | | SEQ ID NO: 860 |
| 414 | AATAAATATAAGATATATTGAGACA | | TGTCTCAATATATCTTATATTTATT |
| | | | SEQ ID NO: 861 |
| 415 | TAATAAATATAAGATATATTGAGAC | | GTCTCAATATATCTTATATTTATTA |
| | | | SEQ ID NO: 862 |
| 416 | ATAATAAATATAAGATATATTGAGA | | TCTCAATATATCTTATATTTATTAT |
| | | | SEQ ID NO: 863 |
| 417 | AATAATAAATATAAGATATATTGAG | | CTCAATATATCTTATATTTATTATT |
| | | | SEQ ID NO: 864 |
| 418 | AAATAATAAATATAAGATATATTGA | | TCAATATATCTTATATTTATTATTT |
| | | | SEQ ID NO: 865 |
| 419 | TAAATAATAAATATAAGATATATTG | | CAATATATCTTATATTTATTATTTA |
| | | | SEQ ID NO: 866 |
| 420 | GTAAATAATAAATATAAGATATATT | | AATATATCTTATATTTATTATTTAC |
| | | | SEQ ID NO: 867 |
| 421 | GGTAAATAATAAATATAAGATATAT | | ATATATCTTATATTTATTATTTACC |
| | | | SEQ ID NO: 868 |
| 422 | TGGTAAATAATAAATATAAGATATA | | TATATCTTATATTTATTATTTACCA |
| | | | SEQ ID NO: 869 |
| 423 | TTGGTAAATAATAAATATAAGATAT | | ATATCTTATATTTATTATTTACCAA |
| | | | SEQ ID NO: 870 |
| 424 | TTTGGTAAATAATAAATATAAGATA | | TATCTTATATTTATTATTTACCAAA |
| | | | SEQ ID NO: 871 |
| 425 | ATTTGGTAAATAATAAATATAAGAT | | ATCTTATATTTATTATTTACCAAAT |
| | | | SEQ ID NO: 872 |
| 426 | AATTTGGTAAATAATAAATATAAGA | | TCTTATATTTATTATTTACCAAATT |
| | | | SEQ ID NO: 873 |
| 427 | TAATTTGGTAAATAATAAATATAAG | | CTTATATTTATTATTTACCAAATTA |
| | | | SEQ ID NO: 874 |
| 428 | ATAATTTGGTAAATAATAAATATAA | | TTATATTTATTATTTACCAAATTAT |
| | | | SEQ ID NO: 875 |
| 429 | AATAATTTGGTAAATAATAAATATA | | TATATTTATTATTTACCAAATTATT |
| | | | SEQ ID NO: 876 |
| 430 | GAATAATTTGGTAAATAATAAATAT | | ATATTTATTATTTACCAAATTATTC |
| | | | SEQ ID NO: 877 |
| 431 | AGAATAATTTGGTAAATAATAAATA | | TATTTATTATTTACCAAATTATTCT |
| | | | SEQ ID NO: 878 |
| 432 | TAGAATAATTTGGTAAATAATAAAT | | ATTTATTATTTACCAAATTATTCTA |
| | | | SEQ ID NO: 879 |
| 433 | TTAGAATAATTTGGTAAATAATAAA | | TTTATTATTTACCAAATTATTCTAA |
| | | | SEQ ID NO: 880 |
| 434 | CTTAGAATAATTTGGTAAATAATAA | | TTATTATTTACCAAATTATTCTAAG |
| | | | SEQ ID NO: 881 |
| 435 | TCTTAGAATAATTTGGTAAATAATA | | TATTATTTACCAAATTATTCTAAGA |
| | | | SEQ ID NO: 882 |
| 436 | CTCTTAGAATAATTTGGTAAATAAT | | ATTATTTACCAAATTATTCTAAGAG |
| | | | SEQ ID NO: 883 |
| 437 | ACTCTTAGAATAATTTGGTAAATAA | | TTATTTACCAAATTATTCTAAGAGT |
| | | | SEQ ID NO: 884 |
| 438 | TACTCTTAGAATAATTTGGTAAATA | | TATTTACCAAATTATTCTAAGAGTA |
| | | | SEQ ID NO: 885 |
| 439 | ATACTCTTAGAATAATTTGGTAAAT | | ATTTACCAAATTATTCTAAGAGTAT |
| | | | SEQ ID NO: 886 |
| 440 | AATACTCTTAGAATAATTTGGTAAA | | TTTACCAAATTATTCTAAGAGTATT |
| | | | SEQ ID NO: 887 |
| 441 | AAATACTCTTAGAATAATTTGGTAA | | TTACCAAATTATTCTAAGAGTATTT |
| | | | SEQ ID NO: 888 |
| 442 | GAAATACTCTTAGAATAATTTGGTA | | TACCAAATTATTCTAAGAGTATTTC |
| | | | SEQ ID NO: 889 |
| 443 | AGAAATACTCTTAGAATAATTTGGT | | ACCAAATTATTCTAAGAGTATTTCT |
| | | | SEQ ID NO: 890 |
| 444 | AAGAAATACTCTTAGAATAATTTGG | | CCAAATTATTCTAAGAGTATTTCTT |
| | | | SEQ ID NO: 891 |
| 445 | GAAGAAATACTCTTAGAATAATTTG | | CAAATTATTCTAAGAGTATTTCTTC |
| | | | SEQ ID NO: 892 |
| 446 | GGAAGAAATACTCTTAGAATAATTT | | AAATTATTCTAAGAGTATTTCTTCC |
| | | | SEQ ID NO: 893 |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Table 2 below identifies additional STMN2 AON sequences:

TABLE 2
Additional STMN2 AON Sequences

| SEQ ID NO: | AON Sequence* (5′→3′) |
|---|---|
| 945 | GGAGGGAUACCUGUAUAUUACAAGU |
| 946 | AGGAGGGAUACCUGUAUAUUACAAG |
| 947 | CAGGAGGGAUACCUGUAUAUUACAA |
| 948 | CCAGGAGGGAUACCUGUAUAUUACA |
| 949 | ACCAGGAGGGAUACCUGUAUAUUAC |
| 950 | UACCAGGAGGGAUACCUGUAUAUUA |
| 951 | UUACCAGGAGGGAUACCUGUAUAUU |
| 952 | CUUACCAGGAGGGAUACCUGUAUAU |
| 953 | GCUUACCAGGAGGGAUACCUGUAUA |
| 954 | AGCUUACCAGGAGGGAUACCUGUAU |
| 955 | GAGCUUACCAGGAGGGAUACCUGUA |
| 956 | AGAGCUUACCAGGAGGGAUACCUGU |
| 957 | CAGAGCUUACCAGGAGGGAUACCUG |
| 958 | CCAGAGCUUACCAGGAGGGAUACCU |
| 959 | ACCAGAGCUUACCAGGAGGGAUACC |
| 960 | UACCAGAGCUUACCAGGAGGGAUAC |
| 961 | AUACCAGAGCUUACCAGGAGGGAUA |
| 962 | AAUACCAGAGCUUACCAGGAGGGAU |
| 963 | UAAUACCAGAGCUUACCAGGAGGGA |
| 964 | AUAAUACCAGAGCUUACCAGGAGGG |
| 965 | CAUAAUACCAGAGCUUACCAGGAGG |
| 966 | ACAUAAUACCAGAGCUUACCAGGAG |
| 967 | GACAUAAUACCAGAGCUUACCAGGA |
| 968 | AGACAUAAUACCAGAGCUUACCAGG |
| 969 | AAGACAUAAUACCAGAGCUUACCAG |
| 970 | UAAGACAUAAUACCAGAGCUUACCA |
| 971 | UUAAGACAUAAUACCAGAGCUUACC |
| 972 | GUUAAGACAUAAUACCAGAGCUUAC |
| 973 | UGUUAAGACAUAAUACCAGAGCUUA |
| 974 | AUGUUAAGACAUAAUACCAGAGCUU |
| 975 | AAUGUUAAGACAUAAUACCAGAGCU |
| 976 | AAAUGUUAAGACAUAAUACCAGAGC |
| 977 | AAAAUGUUAAGACAUAAUACCAGAG |
| 978 | AAAAAUGUUAAGACAUAAUACCAGA |
| 979 | UAAAAAUGUUAAGACAUAAUACCAG |
| 980 | UUAAAAAUGUUAAGACAUAAUACCA |
| 981 | UUUAAAAAUGUUAAGACAUAAUACC |
| 982 | AUUUAAAAAUGUUAAGACAUAAUAC |
| 983 | GAUUUAAAAAUGUUAAGACAUAAUA |
| 984 | AGAUUUAAAAAUGUUAAGACAUAAU |
| 985 | UAGAUUUAAAAAUGUUAAGACAUAA |
| 986 | AUAGAUUUAAAAAUGUUAAGACAUA |
| 987 | CAUAGAUUUAAAAAUGUUAAGACAU |
| 988 | CCAUAGAUUUAAAAAUGUUAAGACA |
| 989 | ACCAUAGAUUUAAAAAUGUUAAGAC |
| 990 | UACCAUAGAUUUAAAAAUGUUAAGA |
| 991 | UUACCAUAGAUUUAAAAAUGUUAAG |
| 992 | AUUACCAUAGAUUUAAAAAUGUUAA |
| 993 | GAUUACCAUAGAUUUAAAAAUGUUA |
| 994 | AGAUUACCAUAGAUUUAAAAAUGUU |
| 995 | AAGAUUACCAUAGAUUUAAAAAUGU |
| 996 | AAAGAUUACCAUAGAUUUAAAAAUG |
| 997 | UAAAGAUUACCAUAGAUUUAAAAAU |
| 998 | GUAAAGAUUACCAUAGAUUUAAAAA |
| 999 | UGUAAAGAUUACCAUAGAUUUAAAA |
| 1000 | UUGUAAAGAUUACCAUAGAUUUAAA |
| 1001 | UUUGUAAAGAUUACCAUAGAUUUAA |
| 1002 | UUUUGUAAAGAUUACCAUAGAUUUA |
| 1003 | AUUUUGUAAAGAUUACCAUAGAUUU |
| 1004 | UAUUUUGUAAAGAUUACCAUAGAUU |
| 1005 | AUAUUUUGUAAAGAUUACCAUAGAU |
| 1006 | AAUAUUUUGUAAAGAUUACCAUAGA |
| 1007 | AAAUAUUUUGUAAAGAUUACCAUAG |
| 1008 | AAAAUAUUUUGUAAAGAUUACCAUA |
| 1009 | UAAAAUAUUUUGUAAAGAUUACCAU |
| 1010 | GUAAAAUAUUUUGUAAAGAUUACCA |
| 1011 | AGUAAAAUAUUUUGUAAAGAUUACC |
| 1012 | AAGUAAAAUAUUUUGUAAAGAUUAC |
| 1013 | GAAGUAAAAUAUUUUGUAAAGAUUA |
| 1014 | GGAAGUAAAAUAUUUUGUAAAGAUU |
| 1015 | CGGAAGUAAAAUAUUUUGUAAAGAU |
| 1016 | UCGGAAGUAAAAUAUUUUGUAAAGA |
| 1017 | UUCGGAAGUAAAAUAUUUUGUAAAG |
| 1018 | GUUCGGAAGUAAAAUAUUUUGUAAA |
| 1019 | AGUUCGGAAGUAAAAUAUUUUGUAA |
| 1020 | GAGUUCGGAAGUAAAAUAUUUUGUA |
| 1021 | UGAGUUCGGAAGUAAAAUAUUUUGU |
| 1022 | AUGAGUUCGGAAGUAAAAUAUUUUG |
| 1023 | UAUGAGUUCGGAAGUAAAAUAUUUU |
| 1024 | AUAUGAGUUCGGAAGUAAAAUAUUU |
| 1025 | UAUAUGAGUUCGGAAGUAAAAUAUU |
| 1026 | GUAUAUGAGUUCGGAAGUAAAAUAU |
| 1027 | GGUAUAUGAGUUCGGAAGUAAAAUA |
| 1028 | AGGUAUAUGAGUUCGGAAGUAAAAU |
| 1029 | CAGGUAUAUGAGUUCGGAAGUAAAA |
| 1030 | CCAGGUAUAUGAGUUCGGAAGUAAA |
| 1031 | CCCAGGUAUAUGAGUUCGGAAGUAA |
| 1032 | CCCCAGGUAUAUGAGUUCGGAAGUA |
| 1033 | UCCCCAGGUAUAUGAGUUCGGAAGU |
| 1034 | AUCCCCAGGUAUAUGAGUUCGGAAG |
| 1035 | AAUCCCCAGGUAUAUGAGUUCGGAA |
| 1036 | AAAUCCCCAGGUAUAUGAGUUCGGA |
| 1037 | AAAAUCCCCAGGUAUAUGAGUUCGG |
| 1038 | UAAAAUCCCCAGGUAUAUGAGUUCG |
| 1039 | AUAAAAUCCCCAGGUAUAUGAGUUC |
| 1040 | AAUAAAAUCCCCAGGUAUAUGAGUU |
| 1041 | UAAUAAAAUCCCCAGGUAUAUGAGU |
| 1042 | GUAAUAAAAUCCCCAGGUAUAUGAG |
| 1043 | AGUAAUAAAAUCCCCAGGUAUAUGA |
| 1044 | GAGUAAUAAAAUCCCCAGGUAUAUG |
| 1045 | AGAGUAAUAAAAUCCCCAGGUAUAU |
| 1046 | CAGAGUAAUAAAAUCCCCAGGUAUA |
| 1047 | CCAGAGUAAUAAAAUCCCCAGGUAU |
| 1048 | CCCAGAGUAAUAAAAUCCCCAGGUA |
| 1049 | UCCCAGAGUAAUAAAAUCCCCAGGU |
| 1050 | UUCCCAGAGUAAUAAAAUCCCCAGG |
| 1051 | AUUCCCAGAGUAAUAAAAUCCCCAG |
| 1052 | AAUUCCCAGAGUAAUAAAAUCCCCA |
| 1053 | UAAUUCCCAGAGUAAUAAAAUCCCC |
| 1054 | AUAAUUCCCAGAGUAAUAAAAUCCC |
| 1055 | CAUAAUUCCCAGAGUAAUAAAAUCC |
| 1056 | ACAUAAUUCCCAGAGUAAUAAAAUC |
| 1057 | CACAUAAUUCCCAGAGUAAUAAAAU |
| 1058 | ACACAUAAUUCCCAGAGUAAUAAAA |
| 1059 | AACACAUAAUUCCCAGAGUAAUAAA |
| 1060 | GAACACAUAAUUCCCAGAGUAAUAA |
| 1061 | AGAACACAUAAUUCCCAGAGUAAUA |
| 1062 | CAGAACACAUAAUUCCCAGAGUAAU |
| 1063 | GCAGAACACAUAAUUCCCAGAGUAA |
| 1064 | GGCAGAACACAUAAUUCCCAGAGUA |
| 1065 | GGGCAGAACACAUAAUUCCCAGAGU |
| 1066 | GGGGCAGAACACAUAAUUCCCAGAG |
| 1067 | UGGGGCAGAACACAUAAUUCCCAGA |
| 1068 | AUGGGGCAGAACACAUAAUUCCCAG |
| 1069 | GAUGGGGCAGAACACAUAAUUCCCA |
| 1070 | UGAUGGGGCAGAACACAUAAUUCCC |
| 1071 | GUGAUGGGGCAGAACACAUAAUUCC |
| 1072 | AGUGAUGGGGCAGAACACAUAAUUC |
| 1073 | GAGUGAUGGGGCAGAACACAUAAUU |
| 1074 | AGAGUGAUGGGGCAGAACACAUAAU |
| 1075 | GAGAGUGAUGGGGCAGAACACAUAA |
| 1076 | AGAGAGUGAUGGGGCAGAACACAUA |
| 1077 | GAGAGAGUGAUGGGGCAGAACACAU |
| 1078 | AGAGAGAGUGAUGGGGCAGAACACA |
| 1079 | AAGAGAGAGUGAUGGGGCAGAACAC |
| 1080 | UAAGAGAGAGUGAUGGGGCAGAACA |
| 1081 | UUAAGAGAGAGUGAUGGGGCAGAAC |
| 1082 | AUUAAGAGAGAGUGAUGGGGCAGAA |
| 1083 | AAUUAAGAGAGAGUGAUGGGGCAGA |
| 1084 | CAAUUAAGAGAGAGUGAUGGGGCAG |
| 1085 | CCAAUUAAGAGAGAGUGAUGGGGCA |
| 1086 | UCCAAUUAAGAGAGAGUGAUGGGGC |
| 1087 | AUCCAAUUAAGAGAGAGUGAUGGGG |
| 1088 | AAUCCAAUUAAGAGAGAGUGAUGGG |
| 1089 | AAAUCCAAUUAAGAGAGAGUGAUGG |
| 1090 | AAAAUCCAAUUAAGAGAGAGUGAUG |
| 1091 | AAAAAUCCAAUUAAGAGAGAGUGAU |
| 1092 | UAAAAAUCCAAUUAAGAGAGAGUGA |
| 1093 | UUAAAAAUCCAAUUAAGAGAGAGUG |
| 1094 | UUUAAAAAUCCAAUUAAGAGAGAGU |
| 1095 | UUUUAAAAAUCCAAUUAAGAGAGAG |
| 1096 | AUUUUAAAAAUCCAAUUAAGAGAGA |
| 1097 | AAUUUUAAAAAUCCAAUUAAGAGAG |
| 1098 | UAAUUUUAAAAAUCCAAUUAAGAGA |
| 1099 | AUAAUUUUAAAAAUCCAAUUAAGAG |
| 1100 | UAUAAUUUUAAAAAUCCAAUUAAGA |
| 1101 | AUAUAAUUUUAAAAAUCCAAUUAAG |
| 1102 | AAUAUAAUUUUAAAAAUCCAAUUAA |
| 1103 | GAAUAUAAUUUUAAAAAUCCAAUUA |
| 1104 | UGAAUAUAAUUUUAAAAAUCCAAUU |
| 1105 | AUGAAUAUAAUUUUAAAAAUCCAAU |
| 1106 | UAUGAAUAUAAUUUUAAAAAUCCAA |
| 1107 | AUAUGAAUAUAAUUUUAAAAAUCCA |
| 1108 | AAUAUGAAUAUAAUUUUAAAAAUCC |
| 1109 | CAAUAUGAAUAUAAUUUUAAAAAUC |
| 1110 | GCAAUAUGAAUAUAAUUUUAAAAAU |
| 1111 | UGCAAUAUGAAUAUAAUUUUAAAAA |
| 1112 | CUGCAAUAUGAAUAUAAUUUUAAAA |
| 1113 | CCUGCAAUAUGAAUAUAAUUUUAAA |
| 1114 | UCCUGCAAUAUGAAUAUAAUUUUAA |
| 1115 | GUCCUGCAAUAUGAAUAUAAUUUUA |
| 1116 | AGUCCUGCAAUAUGAAUAUAAUUUU |
| 1117 | GAGUCCUGCAAUAUGAAUAUAAUUU |
| 1118 | CGAGUCCUGCAAUAUGAAUAUAAUU |
| 1119 | CCGAGUCCUGCAAUAUGAAUAUAAU |
| 1120 | GCCGAGUCCUGCAAUAUGAAUAUAA |
| 1121 | UGCCGAGUCCUGCAAUAUGAAUAUA |
| 1122 | CUGCCGAGUCCUGCAAUAUGAAUAU |
| 1123 | UCUGCCGAGUCCUGCAAUAUGAAUA |
| 1124 | UUCUGCCGAGUCCUGCAAUAUGAAU |
| 1125 | CUUCUGCCGAGUCCUGCAAUAUGAA |
| 1126 | UCUUCUGCCGAGUCCUGCAAUAUGA |
| 1127 | GUCUUCUGCCGAGUCCUGCAAUAUG |
| 1128 | GGUCUUCUGCCGAGUCCUGCAAUAU |
| 1129 | AGGUCUUCUGCCGAGUCCUGCAAUA |
| 1130 | AAGGUCUUCUGCCGAGUCCUGCAAU |
| 1131 | GAAGGUCUUCUGCCGAGUCCUGCAA |
| 1132 | CGAAGGUCUUCUGCCGAGUCCUGCA |
| 1133 | UCGAAGGUCUUCUGCCGAGUCCUGC |
| 1134 | CUCGAAGGUCUUCUGCCGAGUCCUG |
| 1135 | UCUCGAAGGUCUUCUGCCGAGUCCU |
| 1136 | CUCUCGAAGGUCUUCUGCCGAGUCC |
| 1137 | UCUCUCGAAGGUCUUCUGCCGAGUC |
| 1138 | UUCUCUCGAAGGUCUUCUGCCGAGU |
| 1139 | UUUCUCUCGAAGGUCUUCUGCCGAG |
| 1140 | CUUUCUCUCGAAGGUCUUCUGCCGA |
| 1141 | CCUUUCUCUCGAAGGUCUUCUGCCG |
| 1142 | ACCUUUCUCUCGAAGGUCUUCUGCC |
| 1143 | UACCUUUCUCUCGAAGGUCUUCUGC |
| 1144 | CUACCUUUCUCUCGAAGGUCUUCUG |
| 1145 | UCUACCUUUCUCUCGAAGGUCUUCU |
| 1146 | UUCUACCUUUCUCUCGAAGGUCUUC |
| 1147 | UUUCUACCUUUCUCUCGAAGGUCUU |
| 1148 | UUUUCUACCUUUCUCUCGAAGGUCU |
| 1149 | AUUUUCUACCUUUCUCUCGAAGGUC |
| 1150 | UAUUUUCUACCUUUCUCUCGAAGGU |
| 1151 | UUAUUUUCUACCUUUCUCUCGAAGG |
| 1152 | CUUAUUUUCUACCUUUCUCUCGAAG |
| 1153 | UCUUAUUUUCUACCUUUCUCUCGAA |
| 1154 | UUCUUAUUUUCUACCUUUCUCUCGA |
| 1155 | AUUCUUAUUUUCUACCUUUCUCUCG |
| 1156 | AAUUCUUAUUUUCUACCUUUCUCUC |
| 1157 | AAAUUCUUAUUUUCUACCUUUCUCU |
| 1158 | CAAAUUCUUAUUUUCUACCUUUCUC |
| 1159 | CCAAAUUCUUAUUUUCUACCUUUCU |
| 1160 | GCCAAAUUCUUAUUUUCUACCUUUC |
| 1161 | AGCCAAAUUCUUAUUUUCUACCUUU |
| 1162 | GAGCCAAAUUCUUAUUUUCUACCUU |
| 1163 | AGAGCCAAAUUCUUAUUUUCUACCU |
| 1164 | GAGAGCCAAAUUCUUAUUUUCUACC |
| 1165 | AGAGAGCCAAAUUCUUAUUUUCUAC |
| 1166 | CAGAGAGCCAAAUUCUUAUUUUCUA |
| 1167 | ACAGAGAGCCAAAUUCUUAUUUUCU |
| 1168 | CACAGAGAGCCAAAUUCUUAUUUUC |
| 1169 | ACACAGAGAGCCAAAUUCUUAUUUU |
| 1170 | CACACAGAGAGCCAAAUUCUUAUUU |
| 1171 | UCACACAGAGAGCCAAAUUCUUAUU |
| 1172 | CUCACACAGAGAGCCAAAUUCUUAU |
| 1173 | GCUCACACAGAGAGCCAAAUUCUUA |
| 1174 | UGCUCACACAGAGAGCCAAAUUCUU |
| 1175 | AUGCUCACACAGAGAGCCAAAUUCU |
| 1176 | CAUGCUCACACAGAGAGCCAAAUUC |
| 1177 | ACAUGCUCACACAGAGAGCCAAAUU |
| 1178 | CACAUGCUCACACAGAGAGCCAAAU |
| 1179 | ACACAUGCUCACACAGAGAGCCAAA |
| 1180 | CACACAUGCUCACACAGAGAGCCAA |
| 1181 | GCACACAUGCUCACACAGAGAGCCA |
| 1182 | CGCACACAUGCUCACACAGAGAGCC |
| 1183 | ACGCACACAUGCUCACACAGAGAGC |
| 1184 | CACGCACACAUGCUCACACAGAGAG |
| 1185 | ACACGCACACAUGCUCACACAGAGA |
| 1186 | CACACGCACACAUGCUCACACAGAG |
| 1187 | ACACACGCACACAUGCUCACACAGA |
| 1188 | CACACACGCACACAUGCUCACACAG |
| 1189 | GCACACACGCACACAUGCUCACACA |
| 1190 | CGCACACACGCACACAUGCUCACAC |
| 1191 | UCGCACACACGCACACAUGCUCACA |
| 1192 | CUCGCACACACGCACACAUGCUCAC |
| 1193 | UCUCGCACACACGCACACAUGCUCA |
| 1194 | CUCUCGCACACACGCACACAUGCUC |
| 1195 | UCUCUCGCACACACGCACACAUGCU |
| 1196 | CUCUCUCGCACACACGCACACAUGC |
| 1197 | UCUCUCUCGCACACACGCACACAUG |
| 1198 | CUCUCUCUCGCACACACGCACACAU |
| 1199 | UCUCUCUCUCGCACACACGCACACA |
| 1200 | CUCUCUCUCUCGCACACACGCACAC |
| 1201 | UCUCUCUCUCUCGCACACACGCACA |
| 1202 | GUCUCUCUCUCUCGCACACACGCAC |
| 1203 | UGUCUCUCUCUCUCGCACACACGCA |
| 1204 | CUGUCUCUCUCUCUCGCACACACGC |
| 1205 | UCUGUCUCUCUCUCUCGCACACACG |
| 1206 | GUCUGUCUCUCUCUCUCGCACACAC |
| 1207 | UGUCUGUCUCUCUCUCUCGCACACA |
| 1208 | CUGUCUGUCUCUCUCUCUCGCACAC |
| 1209 | GCUGUCUGUCUCUCUCUCUCGCACA |
| 1210 | GGCUGUCUGUCUCUCUCUCUCGCAC |
| 1211 | AGGCUGUCUGUCUCUCUCUCUCGCA |
| 1212 | CAGGCUGUCUGUCUCUCUCUCUCGC |
| 1213 | GCAGGCUGUCUGUCUCUCUCUCUCG |
| 1214 | GGCAGGCUGUCUGUCUCUCUCUCUC |
| 1215 | AGGCAGGCUGUCUGUCUCUCUCUCU |
| 1216 | UAGGCAGGCUGUCUGUCUCUCUCUC |
| 1217 | UUAGGCAGGCUGUCUGUCUCUCUCU |
| 1218 | CUUAGGCAGGCUGUCUGUCUCUCUC |
| 1219 | UCUUAGGCAGGCUGUCUGUCUCUCU |
| 1220 | UUCUUAGGCAGGCUGUCUGUCUCUC |
| 1221 | CUUCUUAGGCAGGCUGUCUGUCUCU |
| 1222 | UCUUCUUAGGCAGGCUGUCUGUCUC |
| 1223 | UUCUUCUUAGGCAGGCUGUCUGUCU |
| 1224 | UUUCUUCUUAGGCAGGCUGUCUGUC |
| 1225 | AUUUCUUCUUAGGCAGGCUGUCUGU |
| 1226 | CAUUUCUUCUUAGGCAGGCUGUCUG |
| 1227 | UCAUUUCUUCUUAGGCAGGCUGUCU |
| 1228 | UUCAUUUCUUCUUAGGCAGGCUGUC |
| 1229 | AUUCAUUUCUUCUUAGGCAGGCUGU |
| 1230 | CAUUCAUUUCUUCUUAGGCAGGCUG |
| 1231 | ACAUUCAUUUCUUCUUAGGCAGGCU |
| 1232 | CACAUUCAUUUCUUCUUAGGCAGGC |
| 1233 | UCACAUUCAUUUCUUCUUAGGCAGG |
| 1234 | UUCACAUUCAUUUCUUCUUAGGCAG |
| 1235 | AUUCACAUUCAUUUCUUCUUAGGCA |
| 1236 | CAUUCACAUUCAUUUCUUCUUAGGC |
| 1237 | GCAUUCACAUUCAUUUCUUCUUAGG |
| 1238 | CGCAUUCACAUUCAUUUCUUCUUAG |
| 1239 | CCGCAUUCACAUUCAUUUCUUCUUA |
| 1240 | GCCGCAUUCACAUUCAUUUCUUCUU |
| 1241 | AGCCGCAUUCACAUUCAUUUCUUCU |
| 1242 | AAGCCGCAUUCACAUUCAUUUCUUC |
| 1243 | CAAGCCGCAUUCACAUUCAUUUCUU |
| 1244 | ACAAGCCGCAUUCACAUUCAUUUCU |
| 1245 | CACAAGCCGCAUUCACAUUCAUUUC |
| 1246 | CCACAAGCCGCAUUCACAUUCAUUU |
| 1247 | GCCACAAGCCGCAUUCACAUUCAUU |
| 1248 | UGCCACAAGCCGCAUUCACAUUCAU |
| 1249 | GUGCCACAAGCCGCAUUCACAUUCA |
| 1250 | UGUGCCACAAGCCGCAUUCACAUUC |
| 1251 | CUGUGCCACAAGCCGCAUUCACAUU |
| 1252 | ACUGUGCCACAAGCCGCAUUCACAU |
| 1253 | AACUGUGCCACAAGCCGCAUUCACA |
| 1254 | CAACUGUGCCACAAGCCGCAUUCAC |
| 1255 | UCAACUGUGCCACAAGCCGCAUUCA |
| 1256 | GUCAACUGUGCCACAAGCCGCAUUC |
| 1257 | UGUCAACUGUGCCACAAGCCGCAUU |
| 1258 | UUGUCAACUGUGCCACAAGCCGCAU |
| 1259 | CUUGUCAACUGUGCCACAAGCCGCA |
| 1260 | CCUUGUCAACUGUGCCACAAGCCGC |
| 1261 | UCCUUGUCAACUGUGCCACAAGCCG |
| 1262 | AUCCUUGUCAACUGUGCCACAAGCC |
| 1263 | CAUCCUUGUCAACUGUGCCACAAGC |
| 1264 | UCAUCCUUGUCAACUGUGCCACAAG |
| 1265 | AUCAUCCUUGUCAACUGUGCCACAA |
| 1266 | UAUCAUCCUUGUCAACUGUGCCACA |
| 1267 | UUAUCAUCCUUGUCAACUGUGCCAC |
| 1268 | UUUAUCAUCCUUGUCAACUGUGCCA |
| 1269 | AUUUAUCAUCCUUGUCAACUGUGCC |
| 1270 | GAUUUAUCAUCCUUGUCAACUGUGC |
| 1271 | UGAUUUAUCAUCCUUGUCAACUGUG |
| 1272 | UUGAUUUAUCAUCCUUGUCAACUGU |
| 1273 | AUUGAUUUAUCAUCCUUGUCAACUG |
| 1274 | UAUUGAUUUAUCAUCCUUGUCAACU |
| 1275 | UUAUUGAUUUAUCAUCCUUGUCAAC |
| 1276 | AUUAUUGAUUUAUCAUCCUUGUCAA |
| 1277 | CAUUAUUGAUUUAUCAUCCUUGUCA |
| 1278 | GCAUUAUUGAUUUAUCAUCCUUGUC |
| 1279 | UGCAUUAUUGAUUUAUCAUCCUUGU |
| 1280 | UUGCAUUAUUGAUUUAUCAUCCUUG |
| 1281 | CUUGCAUUAUUGAUUUAUCAUCCUU |
| 1282 | GCUUGCAUUAUUGAUUUAUCAUCCU |
| 1283 | AGCUUGCAUUAUUGAUUUAUCAUCC |
| 1284 | AAGCUUGCAUUAUUGAUUUAUCAUC |
| 1285 | UAAGCUUGCAUUAUUGAUUUAUCAU |
| 1286 | GUAAGCUUGCAUUAUUGAUUUAUCA |
| 1287 | AGUAAGCUUGCAUUAUUGAUUUAUC |
| 1288 | UAGUAAGCUUGCAUUAUUGAUUUAU |
| 1289 | AUAGUAAGCUUGCAUUAUUGAUUUA |
| 1290 | GAUAGUAAGCUUGCAUUAUUGAUUU |
| 1291 | UGAUAGUAAGCUUGCAUUAUUGAUU |
| 1292 | AUGAUAGUAAGCUUGCAUUAUUGAU |
| 1293 | AAUGAUAGUAAGCUUGCAUUAUUGA |
| 1294 | AAAUGAUAGUAAGCUUGCAUUAUUG |
| 1295 | UAAAUGAUAGUAAGCUUGCAUUAUU |
| 1296 | AUAAAUGAUAGUAAGCUUGCAUUAU |
| 1297 | CAUAAAUGAUAGUAAGCUUGCAUUA |
| 1298 | UCAUAAAUGAUAGUAAGCUUGCAUU |
| 1299 | UUCAUAAAUGAUAGUAAGCUUGCAU |
| 1300 | AUUCAUAAAUGAUAGUAAGCUUGCA |
| 1301 | UAUUCAUAAAUGAUAGUAAGCUUGC |
| 1302 | CUAUUCAUAAAUGAUAGUAAGCUUG |
| 1303 | GCUAUUCAUAAAUGAUAGUAAGCUU |
| 1304 | UGCUAUUCAUAAAUGAUAGUAAGCU |
| 1305 | UUGCUAUUCAUAAAUGAUAGUAAGC |
| 1306 | AUUGCUAUUCAUAAAUGAUAGUAAG |
| 1307 | UAUUGCUAUUCAUAAAUGAUAGUAA |
| 1308 | GUAUUGCUAUUCAUAAAUGAUAGUA |
| 1309 | AGUAUUGCUAUUCAUAAAUGAUAGU |
| 1310 | CAGUAUUGCUAUUCAUAAAUGAUAG |
| 1311 | UCAGUAUUGCUAUUCAUAAAUGAUA |
| 1312 | UUCAGUAUUGCUAUUCAUAAAUGAU |
| 1313 | CUUCAGUAUUGCUAUUCAUAAAUGA |
| 1314 | UCUUCAGUAUUGCUAUUCAUAAAUG |
| 1315 | UUCUUCAGUAUUGCUAUUCAUAAAU |
| 1316 | UUUCUUCAGUAUUGCUAUUCAUAAA |
| 1317 | AUUUCUUCAGUAUUGCUAUUCAUAA |
| 1318 | AAUUUCUUCAGUAUUGCUAUUCAUA |
| 1319 | UAAUUUCUUCAGUAUUGCUAUUCAU |
| 1320 | UUAAUUUCUUCAGUAUUGCUAUUCA |
| 1321 | UUUAAUUUCUUCAGUAUUGCUAUUC |
| 1322 | UUUUAAUUUCUUCAGUAUUGCUAUU |
| 1323 | GUUUUAAUUUCUUCAGUAUUGCUAU |
| 1324 | UGUUUUAAUUUCUUCAGUAUUGCUA |
| 1325 | UUGUUUUAAUUUCUUCAGUAUUGCU |
| 1326 | UUUGUUUUAAUUUCUUCAGUAUUGC |
| 1327 | UUUUGUUUUAAUUUCUUCAGUAUUG |
| 1328 | CUUUUGUUUUAAUUUCUUCAGUAUU |
| 1329 | UCUUUUGUUUUAAUUUCUUCAGUAU |
| 1330 | AUCUUUUGUUUUAAUUUCUUCAGUA |
| 1331 | AAUCUUUUGUUUUAAUUUCUUCAGU |
| 1332 | CAAUCUUUUGUUUUAAUUUCUUCAG |
| 1333 | GCAAUCUUUUGUUUUAAUUUCUUCA |
| 1334 | AGCAAUCUUUUGUUUUAAUUUCUUC |
| 1335 | CAGCAAUCUUUUGUUUUAAUUUCUU |
| 1336 | ACAGCAAUCUUUUGUUUUAAUUUCU |
| 1337 | GACAGCAAUCUUUUGUUUUAAUUUC |
| 1338 | AGACAGCAAUCUUUUGUUUUAAUUU |
| 1339 | GAGACAGCAAUCUUUUGUUUUAAUU |
| 1340 | UGAGACAGCAAUCUUUUGUUUUAAU |
| 1341 | UUGAGACAGCAAUCUUUUGUUUUAA |
| 1342 | AUUGAGACAGCAAUCUUUUGUUUUA |
| 1343 | UAUUGAGACAGCAAUCUUUUGUUUU |
| 1344 | AUAUUGAGACAGCAAUCUUUUGUUU |
| 1345 | UAUAUUGAGACAGCAAUCUUUUGUU |
| 1346 | AUAUAUUGAGACAGCAAUCUUUUGU |
| 1347 | GAUAUAUUGAGACAGCAAUCUUUUG |
| 1348 | AGAUAUAUUGAGACAGCAAUCUUUU |
| 1349 | AAGAUAUAUUGAGACAGCAAUCUUU |
| 1350 | UAAGAUAUAUUGAGACAGCAAUCUU |
| 1351 | AUAAGAUAUAUUGAGACAGCAAUCU |
| 1352 | UAUAAGAUAUAUUGAGACAGCAAUC |
| 1353 | AUAUAAGAUAUAUUGAGACAGCAAU |
| 1354 | AAUAUAAGAUAUAUUGAGACAGCAA |
| 1355 | AAAUAUAAGAUAUAUUGAGACAGCA |
| 1356 | UAAAUAUAAGAUAUAUUGAGACAGC |
| 1357 | AUAAAUAUAAGAUAUAUUGAGACAG |
| 1358 | AAUAAAUAUAAGAUAUAUUGAGACA |
| 1359 | UAAUAAAUAUAAGAUAUAUUGAGAC |
| 1360 | AUAAUAAAUAUAAGAUAUAUUGAGA |
| 1361 | AAUAAUAAAUAUAAGAUAUAUUGAG |
| 1362 | AAAUAAUAAAUAUAAGAUAUAUUGA |
| 1363 | UAAAUAAUAAAUAUAAGAUAUAUUG |
| 1364 | GUAAAUAAUAAAUAUAAGAUAUAUU |
| 1365 | GGUAAAUAAUAAAUAUAAGAUAUAU |
| 1366 | UGGUAAAUAAUAAAUAUAAGAUAUA |
| 1367 | UUGGUAAAUAAUAAAUAUAAGAUAU |
| 1368 | UUUGGUAAAUAAUAAAUAUAAGAUA |
| 1369 | AUUUGGUAAAUAAUAAAUAUAAGAU |
| 1370 | AAUUUGGUAAAUAAUAAAUAUAAGA |
| 1371 | UAAUUUGGUAAAUAAUAAAUAUAAG |
| 1372 | AUAAUUUGGUAAAUAAUAAAUAUAA |
| 1373 | AAUAAUUUGGUAAAUAAUAAAUAUA |
| 1374 | GAAUAAUUUGGUAAAUAAUAAAUAU |
| 1375 | AGAAUAAUUUGGUAAAUAAUAAAUA |
| 1376 | UAGAAUAAUUUGGUAAAUAAUAAAU |
| 1377 | UUAGAAUAAUUUGGUAAAUAAUAAA |
| 1378 | CUUAGAAUAAUUUGGUAAAUAAUAA |
| 1379 | UCUUAGAAUAAUUUGGUAAAUAAUA |
| 1380 | CUCUUAGAAUAAUUUGGUAAAUAAU |
| 1381 | ACUCUUAGAAUAAUUUGGUAAAUAA |
| 1382 | UACUCUUAGAAUAAUUUGGUAAAUA |
| 1383 | AUACUCUUAGAAUAAUUUGGUAAAU |
| 1384 | AAUACUCUUAGAAUAAUUUGGUAAA |
| 1385 | AAAUACUCUUAGAAUAAUUUGGUAA |
| 1386 | GAAAUACUCUUAGAAUAAUUUGGUA |
| 1387 | AGAAAUACUCUUAGAAUAAUUUGGU |
| 1388 | AAGAAAUACUCUUAGAAUAAUUUGG |
| 1389 | GAAGAAAUACUCUUAGAAUAAUUUG |
| 1390 | GGAAGAAAUACUCUUAGAAUAAUUU |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

Table 3 below identifies exemplary STMN2 AON sequences:

TABLE 3
Exemplary STMN2 AON Sequences

| SEQ ID NO: | Oligonucleotide |
|---|---|
| (legacy ID*) | sequence (5′→3′) |
| SEQ ID NO: 31 | AATGTTAAGACATAATACCAGAGCT |
| (QSN-31) | |
| SEQ ID NO: 36 | TTAAAAATGTTAAGACATAATACCA |
| (QSN-36) | |
| SEQ ID NO: 41 | TAGATTTAAAAATGTTAAGACATAA |
| (QSN-41) | |
| SEQ ID NO: 46 | TACCATAGATTTAAAAATGTTAAGA |
| (QSN-46) | |
| SEQ ID NO: 55 | TGTAAAGATTACCATAGATTTAAAA |
| (QSN-55) | |
| SEQ ID NO: 144 | AATCCAATTAAGAGAGAGTGATGGG |
| (QSN-144) | |
| SEQ ID NO: 146 | AAAATCCAATTAAGAGAGAGTGATG |
| (QSN-146) | |
| SEQ ID NO: 150 | TTTAAAAATCCAATTAAGAGAGAGT |
| (QSN-150) | |
| SEQ ID NO: 169 | CCTGCAATATGAATATAATTTTAAA |
| (QSN-169) | |
| SEQ ID NO: 170 | TCCTGCAATATGAATATAATTTTAA |
| (QSN-170) | |
| SEQ ID NO: 171 | GTCCTGCAATATGAATATAATTTTA |
| (QSN-171) | |
| SEQ ID NO: 172 | AGTCCTGCAATATGAATATAATTTT |
| (QSN-172) | |
| SEQ ID NO: 173 | GAGTCCTGCAATATGAATATAATTT |
| (QSN-173) | |
| SEQ ID NO: 177 | TGCCGAGTCCTGCAATATGAATATA |
| (QSN-177) | |
| SEQ ID NO: 181 | CTTCTGCCGAGTCCTGCAATATGAA |
| (QSN-181) | |
| SEQ ID NO: 185 | AGGTCTTCTGCCGAGTCCTGCAATA |
| (QSN-185) | |
| SEQ ID NO: 197 | CCTTTCTCTCGAAGGTCTTCTGCCG |
| (QSN-197) | |
| SEQ ID NO: 203 | TTTCTACCTTTCTCTCGAAGGTCTT |
| (QSN-203) | |
| SEQ ID NO: 209 | TCTTATTTTCTACCTTTCTCTCGAA |
| (QSN-209) | |
| SEQ ID NO: 215 | CCAAATTCTTATTTTCTACCTTTCT |
| (QSN-215) | |
| SEQ ID NO: 237 | GCACACATGCTCACACAGAGAGCCA |
| (QSN-237) | |
| SEQ ID NO: 244 | CACACACGCACACATGCTCACACAG |
| (QSN-244) | |
| SEQ ID NO: 249 | TCTCGCACACACGCACACATGCTCA |
| (QSN-249) | |
| SEQ ID NO: 252 | CTCTCTCGCACACACGCACACATGC |
| (QSN-252) | |
| SEQ ID NO: 380 | TGTTTTAATTTCTTCAGTATTGCTA |
| (QSN-380) | |
| SEQ ID NO: 385 | TCTTTTGTTTTAATTTCTTCAGTAT |
| (QSN-385) | |
| SEQ ID NO: 390 | AGCAATCTTTTGTTTTAATTTCTTC |
| (QSN-390) | |
| SEQ ID NO: 395 | GAGACAGCAATCTTTTGTTTTAATT |
| (QSN-395) | |
| SEQ ID NO: 400 | ATATTGAGACAGCAATCTTTTGTTT |
| (QSN-400) | |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage.

In some embodiments, all internucleoside linkages of the STMN2 AON oligonucleotides listed in Table 3 are phosphorothioate linkages, and each of the linked nucleosides of the oligonucleotide are 2′-O-(2-methoxyethyl) (2′-MOE) nucleosides, and each “C” is replaced with a 5-MeC. For example, in some embodiments, all internucleoside linkages of the QSN-31 STMN2 AON (SEQ ID NO: 31) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-36 STMN2 AON (SEQ ID NO: 36) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-55 STMN2 AON (SEQ ID NO: 55) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-144 STMN2 AON (SEQ ID NO: 144) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-173 STMN2 AON (SEQ ID NO: 173) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-177 STMN2 AON (SEQ ID NO: 177) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-181 STMN2 AON (SEQ ID NO: 181) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-185 STMN2 AON (SEQ ID NO: 185) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-197 STMN2 AON (SEQ ID NO: 197) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-203 STMN2 AON (SEQ ID NO: 203) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-209 STMN2 AON (SEQ ID NO: 209) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-215 STMN2 AON (SEQ ID NO: 215) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-237 STMN2 AON (SEQ ID NO: 237) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-244 STMN2 AON (SEQ ID NO: 244) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-252 STMN2 AON (SEQ ID NO: 252) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-380 STMN2 AON (SEQ ID NO: 380) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-385 STMN2 AON (SEQ ID NO: 385) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-390 STMN2 AON (SEQ ID NO: 390) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-395 STMN2 AON (SEQ ID NO: 395) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-400 STMN2 AON (SEQ ID NO: 400) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-169 STMN2 AON (SEQ ID NO: 169) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-170 STMN2 AON (SEQ ID NO: 170) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-171 STMN2 AON (SEQ ID NO: 171) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-172 STMN2 AON (SEQ ID NO: 172) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-249 STMN2 AON (SEQ ID NO: 249) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In some embodiments, all internucleoside linkages of the STMN2 AON oligonucleotides listed in Table 3 are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. For example, in some embodiments, all internucleoside linkages of the QSN-31 STMN2 AON (SEQ ID NO: 31) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-36 STMN2 AON (SEQ ID NO: 36) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-55 STMN2 AON (SEQ ID NO: 55) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-144 STMN2 AON (SEQ ID NO: 144) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-173 STMN2 AON (SEQ ID NO: 173) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-177 STMN2 AON (SEQ ID NO: 177) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-181 STMN2 AON (SEQ ID NO: 181) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-185 STMN2 AON (SEQ ID NO: 185) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-197 STMN2 AON (SEQ ID NO: 197) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-203 STMN2 AON (SEQ ID NO: 203) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-209 STMN2 AON (SEQ ID NO: 209) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-215 STMN2 AON (SEQ ID NO: 215) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-237 STMN2 AON (SEQ ID NO: 237) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-244 STMN2 AON (SEQ ID NO: 244) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-252 STMN2 AON (SEQ ID NO: 252) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-380 STMN2 AON (SEQ ID NO: 380) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-385 STMN2 AON (SEQ ID NO: 385) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-390 STMN2 AON (SEQ ID NO: 390) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-395 STMN2 AON (SEQ ID NO: 395) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-400 STMN2 AON (SEQ ID NO: 400) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-169 STMN2 AON (SEQ ID NO: 169) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-170 STMN2 AON (SEQ ID NO: 170) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-171 STMN2 AON (SEQ ID NO: 171) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC. In some embodiments, all internucleoside linkages of the QSN-172 STMN2 AON (SEQ ID NO: 172) oligonucleotide are phosphorothioate linkages, and each of the linked nucleosides of the oligonucleotide are 2′-O-(2-methoxyethyl) (2′-MOE) nucleosides, and none of the ‘C′’ is replaced with 5-MeC. In some embodiments, all internucleoside linkages of the QSN-249 STMN2 AON (SEQ ID NO: 249) oligonucleotide are phosphorothioate linkages, and each of the linked nucleosides of the oligonucleotide are 2′-O-(2-methoxyethyl) (2′-MOE) nucleosides, and not all or none of the ‘C′’ is replaced with 5-MeC.

Table 4 below identifies additional exemplary STMN2 AON sequences:

TABLE 4
Additional Exemplary STMN2 AON Sequences

| | Oligonucleotide |
|---|---|
| SEQ ID NO | sequence (5′→3′) |
| SEQ ID NO: 975 | AAUGUUAAGACAUAAUACCAGAGCU |
| SEQ ID NO: 980 | UUAAAAAUGUUAAGACAUAAUACCA |
| SEQ ID NO: 985 | UAGAUUUAAAAAUGUUAAGACAUAA |
| SEQ ID NO: 990 | UACCAUAGAUUUAAAAAUGUUAAGA |
| SEQ ID NO: 999 | UGUAAAGAUUACCAUAGAUUUAAAA |
| SEQ ID NO: 1088 | AAUCCAAUUAAGAGAGAGUGAUGGG |
| SEQ ID NO: 1090 | AAAAUCCAAUUAAGAGAGAGUGAUG |
| SEQ ID NO: 1094 | UUUAAAAAUCCAAUUAAGAGAGAGU |
| SEQ ID NO: 1113 | CCUGCAAUAUGAAUAUAAUUUUAAA |
| SEQ ID NO: 1114 | UCCUGCAAUAUGAAUAUAAUUUUAA |
| SEQ ID NO: 1115 | GUCCUGCAAUAUGAAUAUAAUUUUA |
| SEQ ID NO: 1116 | AGUCCUGCAAUAUGAAUAUAAUUUU |
| SEQ ID NO: 1117 | GAGUCCUGCAAUAUGAAUAUAAUUU |
| SEQ ID NO: 1121 | UGCCGAGUCCUGCAAUAUGAAUAUA |
| SEQ ID NO: 1125 | CUUCUGCCGAGUCCUGCAAUAUGAA |
| SEQ ID NO: 1129 | AGGUCUUCUGCCGAGUCCUGCAAUA |
| SEQ ID NO: 1141 | CCUUUCUCUCGAAGGUCUUCUGCCG |
| SEQ ID NO: 1147 | UUUCUACCUUUCUCUCGAAGGUCUU |
| SEQ ID NO: 1153 | UCUUAUUUUCUACCUUUCUCUCGAA |
| SEQ ID NO: 1159 | CCAAAUUCUUAUUUUCUACCUUUCU |
| SEQ ID NO: 1181 | GCACACAUGCUCACACAGAGAGCCA |
| SEQ ID NO: 1188 | CACACACGCACACAUGCUCACACAG |
| SEQ ID NO: 1193 | UCUCGCACACACGCACACAUGCUCA |
| SEQ ID NO: 1196 | CUCUCUCGCACACACGCACACAUGC |
| SEQ ID NO: 1324 | UGUUUUAAUUUCUUCAGUAUUGCUA |
| SEQ ID NO: 1329 | UCUUUUGUUUUAAUUUCUUCAGUAU |
| SEQ ID NO: 1334 | AGCAAUCUUUUGUUUUAAUUUCUUC |
| SEQ ID NO: 1339 | GAGACAGCAAUCUUUUGUUUUAAUU |
| SEQ ID NO: 1344 | AUAUUGAGACAGCAAUCUUUUGUUU |

*At least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage

### Full Length STMN2 Transcript

As described herein, the disclosure provides a method of restoring full length STMN2 transcript expression in a cell, where the method includes exposing the cell to an inhibitor of STMN2 transcripts that include a cryptic exon or contacting the cell with an inhibitor of STMN2 transcripts that include a cryptic exon. Such an inhibitor can sterically block splice machinery, sterically mimic TDP43 binding, and/or repress premature polyadenylation of STMN2 pre-mRNA, and increase, restore, and/or stabilize levels of full length STMN2 transcript.

In various embodiments, the full length STMN2 transcript comprises a sequence with accession number NM_001199214.2, identified below as SEQ ID NO: 1433.

(SEQ ID NO: 1433)
A TGGCTAAAAC AGCAATGGCC TACAAGGAAA AAATGAAGGA
GCTGTCCATG CTGTCACTGA TCTGCTCTTG CTTTTACCCG
GAACCTCGCA ACATCAACAT CTATACTTAC GATGATATGG
AAGTGAAGCA AATCAACAAA CGTGCCTCTG GCCAGGCTTT
TGAGCTGATC TTGAAGCCAC CATCTCCTAT CTCAGAAGCC
CCACGAACTT TAGCTTCTCC AAAGAAGAAA GACCTGTCCC
TGGAGGAGAT CCAGAAGAAA CTGGAGGCTG CAGAGGAAAG
AAGAAAGTCT CAGGAGGCCC AGGTGCTGAA ACAATTGGCA
GAGAAGAGGG AACACGAGCG AGAAGTCCTT CAGAAGGCTT
TGGAGGAGAA CAACAACTTC AGCAAGATGG CGGAGGAAAA
GCTGATCCTG AAAATGGAAC AAATTAAGGA AAACCGTGAG
GCTAATCTAG CTGCTATTAT TGAACGTCTG CAGGAAAAGC
TGGTCAAGTT TATTTCTTCT GAACTAAAAG AATCTATAGA
GTCTCAATTT CTGGAGCTTC AGAGGGAAGG AGAGAAGCAA TGA

In various embodiments, the full length STMN2 protein comprises an amino acid sequence with accession number NP_001186143.1, identified below SEQ ID NO: 1434.

(SEQ ID NO: 1434)
MAKTAMAYKE KMKELSMLSL ICSCFYPEPR NINIYTYDDM
EVKQINKRAS GQAFELILKP PSPISEAPRT LASPKKKDLS
LEEIQKKLEA AEERRKSQEA QVLKQLAEKR EHEREVLQKA
LEENNNFSKM AEEKLILKME QIKENREANL AAIIERLQEK
LVKFISSELK ESIESQFLEL QREGEKQ

In various embodiments, the full length STMN2 transcript comprises a sequence with accession number NM_007029.4, identified below as SEQ ID NO: 1435.

(SEQ ID NO: 1435)
A TGGCTAAAAC AGCAATGGCC TACAAGGAAA AAATGAAGGA
GCTGTCCATG CTGTCACTGA TCTGCTCTTG CTTTTACCCG
GAACCTCGCA ACATCAACAT CTATACTTAC GATGATATGG
AAGTGAAGCA AATCAACAAA CGTGCCTCTG GCCAGGCTTT
TGAGCTGATC TTGAAGCCAC CATCTCCTAT CTCAGAAGCC
CCACGAACTT TAGCTTCTCC AAAGAAGAAA GACCTGTCCC
TGGAGGAGAT CCAGAAGAAA CTGGAGGCTG CAGAGGAAAG
AAGAAAGTCT CAGGAGGCCC AGGTGCTGAA ACAATTGGCA
GAGAAGAGGG AACACGAGCG AGAAGTCCTT CAGAAGGCTT
TGGAGGAGAA CAACAACTTC AGCAAGATGG CGGAGGAAAA
GCTGATCCTG AAAATGGAAC AAATTAAGGA AAACCGTGAG
GCTAATCTAG CTGCTATTAT TGAACGTCTG CAGGAAAAGG
AGAGGCATGC TGCGGAGGTG CGCAGGAACA AGGAACTCCA
GGTTGAACTG TCTGGCTGA

In various embodiments, the full length STMN2 protein comprises an amino acid sequence with accession number NP_008960.2, identified below as SEQ ID NO: 1436.

(SEQ ID NO: 1436)
MAKTAMAYKE KMKELSMLSL ICSCFYPEPR NINIYTYDDM
EVKQINKRAS GQAFELILKP PSPISEAPRT LASPKKKDLS
LEEIQKKLEA AEERRKSQEA QVLKQLAEKR EHEREVLQKA
LEENNNFSKM AEEKLILKME QIKENREANL AAIIERLQEK
ERHAAEVRRN KELQVELSG

In various embodiments, the full length STMN2 transcript comprises a sequence with accession number XM_005251142.2, identified below as SEQ ID NO: 1437.

(SEQ ID NO: 1437)
ATGGCCTAC AAGGAAAAAA TGAAGGAGCT GTCCATGCTG
TCACTGATCT GCTCTTGCTT TTACCCGGAA CCTCGCAACA
TCAACATCTA TACTTACGAT GATATGGAAG TGAAGCAAAT
CAACAAACGT GCCTCTGGCC AGGCTTTTGA GCTGATCTTG
AAGCCACCAT CTCCTATCTC AGAAGCCCCA CGAACTTTAG
CTTCTCCAAA GAAGAAAGAC CTGTCCCTGG AGGAGATCCA
GAAGAAACTG GAGGCTGCAG AGGAAAGAAG AAAGTCTCAG
GAGGCCCAGG TGCTGAAACA ATTGGCAGAG AAGAGGGAAC
ACGAGCGAGA AGTCCTTCAG AAGGCTTTGG AGGAGAACAA
CAACTTCAGC AAGATGGCGG AGGAAAAGCT GATCCTGAAA
ATGGAACAAA TTAAGGAAAA CCGTGAGGCT AATCTAGCTG
CTATTATTGA ACGTCTGCAG GAAAAGGAGA GGCATGCTGC
GGAGGTGCGC AGGAACAAGG AACTCCAGGT TGAACTGTCT
GGCTGA

In various embodiments, the full length STMN2 protein comprises an amino acid sequence with accession number XP_005251199, identified below as SEQ ID NO: 1438.

(SEQ ID NO: 1438)
MAYKEKMKEL SMLSLICSCF YPEPRNINIY TYDDMEVKQI
NKRASGQAFE LILKPPSPIS EAPRTLASPK KKDLSLEEIQ
KKLEAAEERR KSQEAQVLKQ LAEKREHERE VLQKALEENN
NFSKMAEEKL ILKMEQIKEN REANLAAIIE RLQEKERHAA
EVRRNKELQV ELSG

STMN2 Transcript with a Cryptic Exon

In one embodiment, a STMN2 transcript with a cryptic exon can comprise the sequence provided as SEQ ID NO: 944.

(SEQ ID NO: 944)
ACTTGTAATATACAGGTATCCCTCCTGGTAAGCTCTGGTATTATGTCTT
AACATTTTTAAATCTATGGTAATCTTTACAAAATATTTTACTTCCGAAC
TCATATACCTGGGGATTTTATTACTCTGGGAATTATGTGTTCTGCCCCA
TCACTCTCTCTTAATTGGATTTTTAAAATTATATTCATATTGCAGGACT
CGGCAGAAGACCTTCGAGAGAAAGGTAGAAAATAAGAATTTGGCTCTCT
GTGTGAGCATGTGTGCGTGTGTGCGAGAGAGAGAGACAGACAGCCTGCC
TAAGAAGAAATGAATGTGAATGCGGCTTGTGGCACAGTTGACAAGGATG
ATAAATCAATAATGCAAGCTTACTATCATTTATGAATAGCAATACTGAA
GAAATTAAAACAAAAGATTGCTGTCTCAATATATCTTATATTTATTATT
TACCAAATTATTCTAAGAGTATTTCTTCC

In one embodiment, a STMN2 transcript with a cryptic exon can comprise a pre-mRNA STMN2 transcript. In one embodiment, a STMN2 transcript with a cryptic exon can comprise the sequence provided as SEQ ID NO: 1391.

(SEQ ID NO: 1391)
AGCTCCTAGGAAGCTTCAGGGCTTAAAGCTCCACTCTACTTGGACTGTACTATCAGGC
CCCCAAAATGGGGGGAGCCGACAGGGAAGGACTGATTTCCATTTCAAACTGCATTCT
GGTACTTTGTACTCCAGCACCATTGGCCGATCAATATTTAATGCTTGGAGATTCTGAC
TCTGCGGGAGTCATGTCAGGGGACCTTGGGAGCCAATCTGCTTGAGCTTCTGAGTGA
TAATTATTCATGGGCTCCTGCCTCTTGCTCTTTCTCTAGCACGGTCCCACTCTGCAGAC
TCAGTGCCTTATTCAGTCTTCTCTCTCGCTCTCTCCGCTGCTGTAGCCGGACCCTTTGC
CTTCGCCACTGCTCAGCGTCTGCACATCCCTACAATGGCTAAAACAGCAATGGTAAG
GCACTGCGCCTCGTTCTCCGTCGGCTCTACCTGGAGCCCACCTCTCACCTCCTCTCTTG
AGCTCTAGAAGCATTCAGAGATATTTTATAAAGAAAAAGATGTTAATGGTAACACAG
GACCAGGAAGGACAGGGCAGTTCTGGGGGAGGTGGGAGGGCAGAGAAGAGGTCTAT
GGAAATCTAAAGCGAAGAATTTCTTTTAAAAGGTAGAAGCGGGTAAGTTGCCCTCCT
ATGGGTAGAGAATTTATTCTGTTTCCATATTTAAAATTAGGACTCAATCGTGAGGGGA
GGAAGCTACCTTAACTGTTTGCCTTAAATGGGCTTAAGGGACATTTTGGAAAGTGCTT
TATAACGACCTTTTTTTTTTTTATTTCTTCTCTAGTTTAAGAAGAAAATAGGAAAGGG
GTAAAGGGAAGGTGGGAGAAAGGAAAAAGAAAATTGCAAAGTCAAAGCGGTCCCAT
CCCGCTGTTTGAAAGATGGGTGGAGACGGGGGGAGGGGATGGAGAGAACTGGGCAC
ATTTTACGGTATTGTCTCGTCGAAGAAACCGCTAGTCCTGGGGTGCGGTGCAGGGAG
GTAAGACGGCGGGGGACAGGGTGGGGGTAGGACCTCCGCTCCTTTGTTTTAGGGCAA
GGGAGGGGAAGGAGAGAGGAAGTCGCGGAGGGCGTGGAGGGCGCGGGTGGGCAGC
TGCAGGGGCGGGGAAGCGCGCGGCAGGGAGGGGTGGAGGGACAGCGGCTTCGAAG
GCGCTGGGGTGGGGTTTCTTTGTGTGCGGACCAGCGGTCCCGGGGGGAGGCACCTGC
AGCGCTGGGCGCACAATGCGGACAGCCCCACCCAGTGCGGAACCGCGCAGCCCCGC
CCCCCCGCCCGGTGCTGCATCTTCATTCGAAAGGGGGTCGGGTGGGGAGCGCAGCGT
GACACCCAGGAGCCCAACCCTGCGGGGACAGCGGCGCCACGCCCCGCGCTCCCCGCT
CCCGACTCCCCGCCGCGGCTTCCAAGAGAGACCTGACCACTGACCCCGCCCTCCCCA
CGCTGGCCTCATTGTTCTGCTTTTAAGAGAGATGGGAAAAGTGGGTTAACATTTTTCT
TTTCGGAAGCAAATTACATAGAGTGTTTAGACATAGACACAGATAAAGGGTTCTTTG
AAGACCTTTGATCGTTTGCGGGAAAAGCTTCTAGAACCTAGACATGTGTATGTATAAT
AATAGAGATGACATGAAATCGTATATAAAGCAAAAGAGGTCAAAGTCTTAAGTTAA
GCCACGCGAAATTTCCGTTTTGTGGGTCAGACAGTGCCAAATATCGGCAATTTCATAA
GCTCAGAGAGACAAGACAGTGGAGACACAGGATGACCGGAAAAGATTCTGGATTCA
GGGCCTTCATCCGCAATTGGTCTTGTGCCTTGAGTGCCCACGGTTCTGGCGCTCAGTG
GCCCCGGGGTGAAAAGGCAGGGTGGGGCCTGGGGTCCTGTGGCAGCTGGAAGCACG
TGTCCCCCGGGACTTGGTTGCAGGATGCGGAGACAGGGAAAGCTGCCGAAAGGACTC
CATCTGCGCGGCTCCGCCCTGCCCTACCCTCCCCGCGGAGCCGGGGAGACCTCAGGC
TCCGAGACTGGCGGGGAAGAGGAATATGGGAGGGGCAGTTGAGCTGTATGCAGTCC
TGGAACCTCTTTTTTCAGCCCCGCAGTCCACAACGGCCCGAGCACCCCTTGATGTGCG
CAGACCCCCGGCGTGGCTCTCAGCCCCAGCACCGAGCCCCTCCCAGCCAAGCGGGTG
GCTCTGCAGAAAAGCTGGCTCGAGCCCCGCCCGGCCACACAAAGGCGCGGCCCCACC
CAGCCCGGGCGCGAGACCGCAGAGGTGACCCCCTTCCCAGGGATTCAGGGAGGGCT
GTCTCTTCTCGCCCACCCACGGTCCGCGGAGCTCGGGGCTTTTTTTCCCCCAGCCCAA
GCCCCCCGCCCACCCTCTGTTCTCTATGATTTTCCAGAATGGAGACCCCGCGAGGGGC
TTCTCTAAGGGAGACCCTCGCTCCTCCAGCGGGGCGCGGCTCGGCCCCACCCCTCCCA
GCTGAGGCCCAGAGCCGCCTACCGCTGGCCGGGTGGGGGCGCACGTGGCGACTGGGT
GTGTGGAGCGCAGCCAGCCCTGCAGAGCCCCGCGCCGCGCCCTGCGCTCCCCTCCCC
GGAGTTGGGCGCTCGCCCCCGCGGTGCAGCCGGGGAGACCGGTTTCTGCGCAGTGTC
CTGAGCTACCCCCGCTTTCCACAATTCGCAGTTCACTCGCACGTCCAGAAAGGTTCTG
AGAATGGGTGGTGGGGGCGATCTCGCCTCGCTTTCTGCACCCCTCAGAAAGGTTTCC
GCTGCAGGCTAGTGGCTGCAAACTCATCGTCATCATCAGTATTATTATCATTTCAAAT
CGTTGTTATTATTTAATGATTCAGTAGCCTTGTTTGTTCTCATTTGTTCAAAAGGGACG
TGGATTGCTCTTGGTTAAGGATTAACCCTTGTTGCGTTCGCTTTGCTTCCTCCTAATTG
CCCTCATCCCTTTCCCCCACAAAAAGGTAAATTTGTCTCCAGTTGTTCATTTTAAGTTA
TAAAGCAAATATATTTTTGCTTCCTGCCAGGATTATGTATGTTCATGTGGCTAAGATA
CATGTGCAAGTGCTTGCTAAGAGCAGGGTTTGTGTGCCAACGATTGCTGGAAAATTC
TCTGCAAAGAATTGTTTGTGGCTGCAATGGGTGAGAATACACATATATAATTGAGAT
GATCTTCAACATAAGGTTATATCTATAAATATATAAATATAGTTTATGCACAAAATTT
TAAGTTTTTTCCCCTGAAACTGTTCTTCCAACTGCTGATTCTTGATACAGCCTCAATCC
TACACAGATACATGGATCGTGAAATGGTAGCCGCCATCCAAATAAAAATCCCACCCC
AAATATGACAAACGCAAGCATCCTTTCTGGCCATAATTTAACTGCATTTGCAAATCAT
GAAAAAAACACTACTTCTGCAGTATTAAAATAATAGATTTTGAAATTAATTCCAATTT
CAAAGATAATTAATTATCAGGGCGAGTGCTTTTTTCCTGATTCATTAAACAATTATGT
ATTCAGCATGATTGTAAGAGGTGCATATAATATTCCCCATTATCTTTTCTAATGAAGT
GGGCACCTTCTGAATGGATATATAAGTAACTAGAAATGAAAAGCTGAGGATTTGGTC
AGAATTTCAGGATAAAACTGAAAGAAATGGCAGTAGTTTATCAATTAATCTCATGTA
TTTAGTTTATACCAGGTGAGTAAGCTGAGCCTGCAATAAACACTCTCTGTCCCAGTGT
AACACGTCGCAGGTAGCTAGAATGATAGGATAAATTAATAGACCTTGTGGTGTTTGT
CTATGCACGTTAAAATTCTCTGAGAGAAAGTATATTTTAAAATGATAATTAAGATTGG
ACATTTGTGCTATTAAAATCTACAACTTTAGTCAAAATTCACAATGGTTTTTTTTTACA
ATAATGTGACTTACAGATTTGTAGTAAATTATTCTATTCTAAAAGAGAAATGAGTGTT
TTTATTGTTACAGCTATTACCTCATTAATATTTTTAGCAAACTTTTATTTGTTGCATTG
AAAGCAGTTTTAATTACTTTGGGTTTTTATTTTTCAAATTACTAATGGATAGATGGTG
GAATAAGCATTTAATCATTTGGCACAATATGACTTCCATCAAATAGCTCATTCTCAGT
GATTAAAAAATGCTACAAGAGGCTACAATTTACTCAGATTCAGGAAATGTCCTTTCA
GAGTGCCATAAGGCTGATTCATATAATAAAATAGTTTTCTTCCCTATAATTTAAGATC
AAATAGTTACTTAGTTCTGTGAATACCTAGCAGTAGCTATCAAACAGAATTTTAAAGT
TAAATCTGTACAACTAACAATGAAGTGGAGGATGAATCGATACATATTGAATGGAAG
ACTTTGTCATTGATAAATTCAGGCCATCTTTAGGAAAATTCCGGATTTATCAATCACC
ATTATTTTTTACTTCAACTGAGTGTGACTGATCACATGCTCAGGCTACCTTGGTAGCT
CATTGCTCACAGGAGGCTGAAAAAAGCTGGCCTCCGAGCAGGAGGAAGCTCAGAGC
ACAAACCTAGGCCTGGGCGTGGCCACTGGGAGCTGCTGATAGCGAACCCCAGCTCAC
ACCAGTTTCTTTTTTGGTCGTGGGAAGAAAAACACATATTATCCTGTTGTCACAAGAT
CTGTGACCTTATATGAAAAAATGCTAGAATTTTTTCATTAAAAAAGAAAATACTGAA
CTAGCCAGTGACCCAGATGTTTTCAGAACCTAGACTGGTTCTGTCCATTGGAAAACCT
CGGTGTCTGCATTAACTTTTCACCACACTAGAGGGCAATCATGTTCTCTAAAAAAGCA
GATGATTGATGTAAACCTAGTTCCAAATATTAACTGTTTAATAAAATCTTTTCTTTTAC
CAGGAACATTCAAGTGTTTATTCAATAAGCTGATGCCATGCTTTACCCTAGTGGATGA
ACAGAGCTTGTACAATTTTCAAGGAGACAGGATGAAATGAGTGGTCATAATCTGAAA
GTAGATACACGCCCTGGTTAATTATTCCCTGATGGTTTTACTTCTCAGTTTTATTACAT
TGTTATTATAATACCATTTATGTTACTTCTGAGATTTTGTAGTGGATAAATAGTAGAA
AAATGTCAGTAGTAATAGCAAAGTTATTTAGCAGCCGAATATTTTAATGCTTAAAAA
TAAAGGAATAAATTAAAGAAAATCATTGTTTACTTCTTCATCGATTGAAATGTGCCCC
CTGTTCAGAGCACATCTGAATATCAGAGTCTCCACCTGCAGAGAACATGCAGCTTAG
CGAGTAAAACAGGCAGGTATGTGATACTGAGGAGGTGTACCAAAAACTGACTGCTGT
TATTTTTCCCATCTTCTAAGTCTGTCTTTCTTTTCCATTTAAAGATACCTTTTTAAATCT
AATCCAATGTGATTTCAATCTAGTTTTATCAGATTTCAACAATTATTGAGCATCTCCTT
GTAGTGGTTTTCTGTTTATTAGAAAATCGATGTTAATTTTAACGAAGTAAGAAGAAAT
ATATAAGTATAAACTAATTTTGGGTATCATCAAAAGTGGATTTTTTAAATATGCATTG
ATAGAATTATTTTTTGATTACATTTTATGTAATTCTAATCCAGCTATAAAATATTTAAT
AGTGTCATATTACTGTGTTCCTCAAACTTTGATGTGCATATGAATTACCTTTGATTTTC
ATTAAAATGCAAATTCTGATTCAATACATCTGGCTTGAGGCAGACATTCTGTCTTCCG
AACAAGCTCCCAGATGATGCTGATTCTGACCACTAAACACATCAGTTTTAGGGATATT
AACTTGTAATATACAGGTATCCCTCCTGGTAAGCTCTGGTATTATGTCTTAACATTTTT
AAATCTATGGTAATCTTTACAAAATATTTTACTTCCGAACTCATATACCTGGGGATTT
TATTACTCTGGGAATTATGTGTTCTGCCCCATCACTCTCTCTTAATTGGATTTTTAAAA
TTATATTCATATTGCAGGACTCGGCAGAAGACCTTCGAGAGAAAGGTAGAAAATAAG
AATTTGGCTCTCTGTGTGAGCATGTGTGCGTGTGTGCGAGAGAGAGAGACAGACAGC
CTGCCTAAGAAGAAATGAATGTGAATGCGGCTTGTGGCACAGTTGACAAGGATGATA
AATCAATAATGCAAGCTTACTATCATTTATGAATAGCAATACTGAAGAAATTAAAAC
AAAAGATTGCTGTCTCAATATATCTTATATTTATTATTTACCAAATTATTCTAAGAGT
ATTTCTTCCTGAATACCATGTGAGAAAATTCTTAAGAATTTATTGAGTATGACTGTAT
ATTTGAAAAGAGTGTTTTCTTCTGCTTATCTAAGCCAATAAAGGATCTTCATTATTCA
ATTCTAACTTTCTAAGGAAGTCAACCTACAGATCAGAAAGAGGATCTTCAAGGAATA
GCATCAAAGACATAGTCAGGTCTCCCATGCAGTGACTGGCTGACCATGCAGCCATTA
CCACCTTTCTGGAAATATTATGCTGCAAAAATGATACAATACACGAAATATCTCAAA
TTAAAAAATATAACATTTCCCAAATAGGGCACTAAAAACATGATCCCAAATAAAACT
AGCTTCAGGGTTTGCAGAATATACTGTTACTCAACACAAAGTTGGACTAAGTCTCAA
AGTTAGCCATTCAGTTGTTGTTAACAGTTCATTTCAGGGTCTCTCAGAAGCTGGGAAA
CTTTCCATTTTTGCAATTTCTTGTACATTGAAGGAAAGGAAGACACACTTAAGACAGC
ATTACAAAAGTAATTCATGTTTTAAATGTTTAATTCTGGCAGTCGGGCAGGGCTCTCT
GTATAACCTCATTTGGAGATGACAAAAATCTAAACTTGAGGGCCTCGAGCCAATAAG
TCTTCCTATTTCTTTACTCAAACATTTTCCCGCAATGGTGCTTTCTTTCAACTGTTTTTC
TGGTGTATTCATAAATTCCAGATTCTCTATGGGAAGTAACTTTTATTGATTGATTTAA
CCCTTGTATAGCACATATAACATGCAAGGCATTGTTCTAAGAACTTTCCACATATTAA
CTGTGTTAATCACTTAATAATCCTAAGTAGGTTCTATTACAGATATGGAAACTGAGGC
ACAGAAAGTTGAAGTATCTTACTCAAGGTCACACAGTTAGTCAGATCCAGAATTTGG
GCCCAGGCCATCTGGCTTCGGAATCCATCTTTCACCGATTGCTGCTAGTCTCATATCT
GTTCCATGTTAGAGGTGAGCTCCCATTGCAGAGGTCACACCTGTGATATCACCATTTT
ATTTAAACAGACCAGAGATGGTCTTCTCCTTTCTGATCACAGACTCACCTTGAAGAGA
AAATACTTCCAAATTGATGCCTAGTTTTAATAGCTTACCTGGGGCTTATTCAAATAAT
TGCCATGATTTAGGCTTTGGGAGAAAGAGAGCTATGAGGCCGTGTGGGTTGTAACGT
ATGAGACACATGGCGTTCTGCAGGCTCAGCACAGCATCGATTTCTGGTGGGAACACA
CTCTGATGACCAGTTCCAGAAATAACATTGACTTAATCTCCTCAGTCCCATCATGGTT
AGCACATTTCAAAATGCCTCCTTAACTACTTCCATAGGCCAGAGATATTTAGTTTTAA
CATTTTGTTGAATAAAATAAATTTACACATTCACATTTAATATAACTATTAGATGTTA
TTTCAAGATTCTCTTCATATTACCATCAAAGCAGGCAGGCAGGCAGGAGAGAACTGT
AGGAAGGTTTTGAATCCCTTGTGAAACATTTTTAATTATCTTTTAATAAAGGAATCAG
GCCCTGTCATTTGTCAAGGAGACATTTGCAGTAGTAAAGCTTGTGTTTATAATATCCA
TTTTTATTAGTCATGATTAAAGATAACATTTGTGTACATTTGTTCTCACAAAACACTTT
TATATGAGTGTAAAGGTTAATTAATGCATTTCAGCCATCATTTTGCTGGTCATGTGGA
AATATAGCTTCTTTAGGAATTGTACTTAGAGTAGGAGCCACATATTATACTATAAAAC
CATAACAAAAATATTTTAAGTTTGTTCTCACTTGTTGTTGACCTCCAGAGTAAAATAT
TTAATACTCTGGAAAGTTATGGGTTTCAAAATTTATTTTATGGCAAGAAATAGATAAT
TACAGTTCTCATAGAGCACATTTAAAATAATTTATTTTTATAGGGCAAAAATATTGCC
TAGGACTGAATGATTTTTTTTTTTTTACAAAGATTGTAAAGCAACGCCTGCAAGAGTG
CCCATTTAGCAGTTATTCTTCTGGAATAATTGTATTTTGGATGTTGGAGTTCGCACATT
AACCATTAGTACAAGTACCCAATATAACAATAGATCATCAGGATAATAAATCTGTCC
ATCTTTTAGTTGTATGTCTTTATATCAGGATAAAGAGAATTGAGTGAAATTTATCTAA
ACCTAGTCCCACAAATACTTTTACAAGAGAGCATGTTAAAGTGTAAATTAAATTTTTA
TTAGCATTCTACTCTGTCTTTGGAAGTTTTTTTTCCTTATGAAATGCAGCCATAAAGTT
TAACTTCCATTAACAAAGCTGCTCACAGTAAACCTATTATAATAATAGTTTCCCAGTT
TGGGCTTCCTAGTGAGGAGCAACCTAACTCACACGAAACAACCCCAACTTATAATAT
ATTGACTGTTACAAAACTGAGACCAGAAAATCCCATCAAGATGGTACTGTTATCATTT
CCAGACTCTCGGGAAGAACATTAATCATCTCAGGCACTTTTAGGATAGACTTATTGCA
GCCTCCCTGGGAACTCTGCTTCAGAACATAATTATTTTTATTAATGCAGAGTTACTTTT
TATTTCCAACAAAAATATCTATTGTTATTATTTAAGTCTTACAGCTTTATCTGAGAAAT
TCCAATTAGCACCCTTCTCATAATAAATATTCAAACACATGAAAAATTACCAAAGTTG
TTCTAGTCTTTTAATGACATATTACATGATCCTGCACTCTTGTCACTTTAAAAATTATC
TTTTTATTATATTTCTGATGATTTTTTTCTTATATAGTTTTTTAAAAGGAGCAGGCAAG
CATAGAAGACTAAAAAATGTTCAAAAGAAAAATTAAATCGCATGATCTATCTATATG
GGACCTTGTCATTTTTAGAAAACATTCACCTGCTTCATCCTTTTGAATCTTCATATAAT
CCCTCTGAGATGGGCATACTATACAAGTTGTCTTATTTAAAGATTGGTAAATTTAAGC
TCAAATAATTTATTCAGTGGCAAGCCTCAGAGGCAGACTCGGAACACAGGTCTAATA
TATATTATATATATATTATAACATATAATATATATATTACATATAATAAAGTTGTGTA
TATTATTTACCTATCAAAATATTTATATGTAATATATAAATATGTTATATATCATGTAT
GTGCCTATTTCATACATATATACACATTCATGCAAAATAAGGTTTAGCACTCCCTCCA
CTGTCCTGTAATAAAACATGCACAGTGAGAATAGTCATACACGAGGCATATTTGTCTT
CAGTTTAAAGTCATTGATAGTCAGTGTCACTAACTAAAGTAAAATAGATTGGAGCAC
CAACTTTGTTCTGAAGCCTGTGCCAGGTATTATGAGAACAAAAATAAAAATGTTCCTC
ACCCTTGGTGGATTTAGTCTTTTGCAGAAAAAAAGATCCTGTACATGTCAGAAAGTTC
AATAGTAATAATGGTAATTTATAACTATAAATGGAAGTCACCATCTCACAATTTCACC
ATCTTAACAATTTTGTTAAACTGCCCTACAATATTACAAGATAGTACATAATGATACA
CTAGTAACATCAACTAGGAAGTACCAAGATCCACCAAAAGGCTGAAAAATTTAAATA
TTTAATGAGTCCATCAACCAATCTGGCCAGAGAATTCTTTAATTAAAATGCTTCCCAA
ATTTTACTGAGAATCAGCAGCGTTTGAGGAGCTAGCCTCCACCCCCAGAGGTTCTCAC
TCTATTAGGTCTGAAGCAGGTCCCATGGATTTGCATTTCTAACAAGCTCCCAGGTGGT
GCTGATGAGGCTGATTCAGAACCACACTTGGAGTAGACCTAAAACAGCAGTGACCTG
TAGGGTCCCCAAGCAGCAGGCCAGGACAGCATGTGAGTTACGTCCTCTGTGGAGCTC
TGCAACAAGGCGTCAAGAGGTCAGAGTCTAAGTCCCCATCAGCTCTGCCCTTCTCCA
CCAGTGCTGCTGGTGCTGCATGGAAGGAAGAGCCCAGAAGGGATTCTGAGTTTCAGT
CTTTACTCTTGCTGACGCACCTTGGTCAGGTCAATTTTCCTGTTTGTTCCTCTAATTCA
GCATCTGTAAAATAGCCATGTGAACTGCCTTGTCCATATCAGAGGGTCTTTTTCAGAC
TCAAGGAAAAAAACGTGAAAGTGATTAGTGTCTGTCAAGTAGTATATAAATGCAAGA
AGTTGAGTTTTTAAATTGTCATTAGATATAAATACCCATGTGCATGCATTTAGAATGA
GTAAAGAGGGAACAAGGAGCGCAATCAAAAACTGCGTCATTTGCTTTTTGAAAAATA
CTTTCTATGTAATGAAAAGTGAAATAAAATGTTAATTGAGTCCCTCTGACAACAGCAT
CAGACGTTTTGCAGTTCTTGTGATTAGAACCCACCTGGCCAGCCCTTCTTCCTCCTAA
AGAAGAGCCTTCTTCTTCTTAAATGAAGGTTGGCTCAGAAGAAGCAATTAACTCATTC
AACGTTTTGTTACAGTCAATCCACATCCAACTTTTCCCCAACTCAATCTGCTTTAAGG
GAAGGATGGTAAGTGGTGGCCCAAGATGGCAACCATCAAGCTTAGAGAATCTCTAGA
AGCAGGGGTGTCCCCAGCAAGTAGACACTGAAAATATGAGAGGGCTGATAAGCCAG
AGATAAAACTCAGTACTTACTTTGCTTCTAGTCCATGTCTACCCCTTTCTTGGCACCAC
CTTGACACTACCCTCTGAGTCCACCTTCCTGAGATGGTACAAACTCTGCTTAGACAAA
GCAGCCCATGTCCAAAGGTGTTAGGGCTCAGTTTAAAGCTGCCTTCAAAAGTTAAAA
CAGAAGTGTAAAGTTCTGTGCAATTAAAAATAATCAGCTTGTCTTGGAACTCAAACG
AATGTAAAATCCTATGAAAATTAAAAAGCAGTACCACAAGTTACCCCAAAAGTCCTT
AGGTCAGTAACTGTTCCTGTTACAGGTAAGAGAGAGCATGGATTAGAGGTGGGCGTG
GGTATCCAGTGGACATGGTTTTGAACCATGCTCCACTACTACTCACTATCTGAGAATT
CTTAAATTTATTAATCATTTCTATATTATAATTTTCTCAGTTATGAAATGGGAAAACA
ATACCTAAATCACATGGTTGTTAAGTAAGCAATTGATTGTTAAGCATTTGGTCATCAA
AAATATTAATCCCCTTCCCTGATTCCCTAGATAAATGATGAAAATACTAAATAAAAAT
AATAAAAATTTAAAGTGAACATCTCAATTCTTATACTTTGTTAATTTCTACATGTATT
ACAAATCTACTAGAAATTACTTGGAATTGAGGAAATGATTACTGCTTAATAATTCTTT
GTGGTAGAGGGAGAGTTGGTATCATATTTATGAGACAGCAGCCAATATAGTATATCT
CAAAGGAAAAAATCCATTCTACATAATGCCAGAATTTAATAGTTAAGCATTTTATCTA
GGTCACAGCACAATAAGCAAGATGGATAATTAAAATAAAAGTATATTTCTCTTGCAT
ATATTTCTCATTTCATGTTTCCCTATCATATTTTATATCTTACCTTACTTCAAATACATA
TATACCTTCAATAAAACTGAGCCTTCTTGCTTACCCAGGAAGTTTCATCATTCAGTAG
AAATAAAAGATGACTTTAGAAATATTAAAATACAAAAATCTACACTGAGGTCTTTTG
AATGCAGGAAAAAGAATTATATCACACACACACGTACACGCACGCATGCATACACAC
ACACAGAACCTCTCGTTCTTTCTTAACATCTTATCAATCCATCAGTTTCACTCCCACTC
CGTATCACCTGACTGTGCACAATATCTCATTGCCACCTCCCAGTCTTCTCCCTGCCTG
GCACCCTCCTGCTCTCCTGCTTCCACTTTAAACACCCTTCCTTCAGCTAGGTCTTTTCT
TTCAGGGATCCTCCCGTTGCTTTCTTATCTGGATCAATTTAGCCTTCCTCTTCTCCACC
CATTAGTGGATAAGCACGACAAAGACACTAGAGTCAAATAATACAAACAGAATATA
CCTTAGATGAGTATGGTGATGAAAAGGATATGGATACTTAGAGTTTAGCACTATTCTC
TCAGCCACTCAGGAAAGCAACGCCTTTACAATCAATAGTGTTTCAGGTACCAATCAA
TAATCTGTTATTGCTATTTTTAAAATCTATAAGGTATCAGTAAAATGTAATTACTAGA
GCAACAAAGATATCTTGTGAAATCAAATTAGTATTCATCCAGCAACTGAGTACAAAG
GTTTAAGGGAGGATAACTACCAATACCAAAACATTTTAAGCATTTTGTTTTGCCTCCT
AAATATCAAATCATGTAAATGTGTGGTACATAAATTAGGAATTATATTTATGACATA
GCTGCAGACATATTAAGAGAAATATGTGCTTATATTTACAAGTATAGTACAGTTCTTT
TTCATATTAGATACTGTTGATGATAATCTGCATATAAAAATGCTCAATATTTTTTCAC
ATTTATAAGCCATAAAATACAGCTAATAAAATGTGTTTCTACTTTCTCATAAACATGG
AATAGTGACAAACAAGGAGCTTTATATGAAAGCACCATTACAATTTAAACTCTCACA
AGGTCATAATATATTGCACTAAGCAGGAGAGTTCAGCTTATTTAAAAAAAAAAATAA
ACTCTAATGAGGTTCTGGAATGCAGAGCCAAAGCATAAAGATGGAAATAAAAGAAT
TGCATGTCTTCTGAACTGACTTGGTTGATGATTTTTTTAAAAAAGGTTTTGTGTCTTCT
GACTTGGTTGATGATTTTTTAAAAAAACGTTTTGTGGTAGAACAAATAAGGTAAATG
AAATTCAGTATTTAGGATGAAAAGTTTTTCTAATTTCAGGAACAACATTGAAGAAAT
ATTGAACTAAGCAGCTTTGAAAGAATCAGATTCCATTTGTTGAAATTTTTCTGAGAAT
GAATTTTTTTAAGACAGTGTACACAGTTGCAGTGTGTATTGGTTATGGATTGTGGCAA
GCTATATTACAACTTACCCAAGAAATAAGGAGGCTGGGCGTGGTGGCTCACACCTGT
AATCCCAGCACTTTGGGTGGCCGAGGCGGGCGGATCACGAGGTCAGGAGATCGAGA
CCATCCTGGCTAACACGGTGAAACCCCGTCTCTACTAAAAGTACAAAAAATTAGCCG
GGTGTGGTGGCGGGTGCCTGTAGTCCCAGCTACTCGGGAGGCTGAGGCAGGAGAATG
GCGTGAATCCGGGAGGGGGAGTTTGCAGTGAGCCGAGATTGTACCACTGCACTCCAG
CCTGGGCGACAGAGCGAGACTCCGTCTCAAAAAAAAAAAAAAAAAAAAAAAAAGA
AAGAAAGAAAGAAGGAAAAAAGTCACTTGAAAAGAATACTGGACTTTGTGTCCAGC
TTGCATAGCTGAAAAGAATAAAAACCTGTCCACTTAAACTCATTGCAAAAAGAAGAT
GTCACTCCTACAAATAGCAAAGAGTCATGAAATTATTCTATCCAGAAAAGTATACAT
TTCATCCCTTTGGATAAATTTTAGAAGTGAACTATGAATACATACGGTGAGGATAGCC
AGCTAAGAAGTCAAGAAGGATTTCTCAAATTTGCTGCTCAGAAAGATCATACTCTCC
ACAAAACAAATAATAGCAGGCTTTCCAAGTCAACCTTGAATCCAGCTTTCCTTTATCT
TTCCTTCTTGTGAACTTTCACTAGTTTACTATCTAACAATGAATTTGACGATAGCCAC
ATACCATCTTATAGCAATATTTGTTATCATATCCCTTGTTATTTATCATTCACCTGCTC
TGCTTGAGCCAGCTACAAGTCACATGTCCCACGCACTTTTTCCTGTTTGATTTTTTACA
GCACTTTGAGACATGTCTCATTATTCCTACTTGACAGGAAAGAAGCCATGGAAAGTT
GAGTGACTTGCTCCTGATCACAAATGCTGGCCAAGGAAGAGTCGAGTTTCAAATCTA
ATGATCTTTCCACTGCACTCTAGATTCCTCATTTTGAACTATTTTTTTATTTTTTGCACT
ATAGACTTTTTTCCACATTTTGAACTGTTTTTTATTTTTTGCACTATAGACTTTTCTCTT
ATACCCAACTATATTGATGACTTCTTTTAGGCTAGAAACTTGTTTCACTTACTTTCCCT
TTCTTCAGATTGCTGCAATATTGGCCAACATGTATTGGGTACTTACTGAGTCAAGTAC
TGTGATTGTGCCAAGTATCTTATAGGAGGATTATCATCCTCATTTTTACAGGTGAGAA
AGGAAAGGAGGTAAAGTCACACACAGCCAACAAAAATGGTAGCACCAGGATTTGAA
ACAAATCAGTCTGACCCAAGTTGACTTTGTTAACCACTGTATGCACAGTCTTCTTAGA
CATAGTAAGAGCTCTAATTGTGTTTGGTGATTTGATTATTATGACAAAGTAAGTAAGG
GAAGCAGGGAGAATTATAAGAAATAAGGCTCCACAACACTTGGCTATAGCAAAGCC
CCTTAAAACTTCAAAAGGTCACCCAAAGAATAAAGATCAGGCTGGGAGCAGTGGCTC
ACGCCTGTAATCCCAGCACTTTGGGAGGCCGAGGTGGGTGGATCACCTGAGTTCAGG
AGTTCGAGACCAGCCTGGACAACATGGTGAAACCCTGTCTCTACTAAAAATACAAAA
ATTAGCTGGATGTGGTGGTTGCCGCCTGTAATCCCAGCTACTTGGGAGGCTGAGGCA
GGGAGAATCGCTTGAACCCAGGAGGTGGAGGTTGCAGTGAGCCGAGATCATGCCACT
GCACTCCAGCCTGGGCAACAAGAGCAAAAAACTCTGACTCAAAAAAATAAATAAAT
CAATCAATAAAATAAAGATCAATTTGGAGAAATTAATGCTTATTAATAAGCAATGTC
TTGCACAGCACTTCAGTTTCTCAATACATTACCTAACTCAATCCTTACAACAACACCC
TATCCCCATTTTGTGGATAAATAAACTCATGTTCAGAAGGTTGAATAAATTATCTAAG
GTTAATAGTTCCTGACCTAGAGCTCAAATCTTCAGTTTCTATCATATTCTTGCCCTTAC
CCTGGGGTAGCTAACATTCACTCACTAGTATTGGAGCTAAAATAAGGGAGAGAACAT
ATAAATGAATACAAAGGAGACATTCACCTGCCTTCTCTTTCTCCTTACATAGAGAAGG
TTGATTATCTGCTATTGTGAAGTTTGCCTTTTGAAGGATAGAAATGAGAAGACTTTCT
TAAATTTTGCCTCTACGCCAAGAAATTAGAGTGGTACCACCAGTAGTTCCATTTTCAA
ACTATCACTGTAGCTAAAGCTATGTGGTAAGGGCCAAGGAAAAGAAGTATTCTTGCA
CTTCAAAATGCACTGAAATACCAGTCAGTAGCATAATATAAAGGAATTTAGTGGAGA
GAAGAGTTGACCTCAATCTGGCTCCAACATCTCGGCTCTTAACCCCTACCCTACACTT
GTTCTTCATGGGGAAGCTAATTGGGCCACTGGAAGATTCAGCAGCTACCATTTGCAG
CTGAGGGACAGCCCCTCCCTGCTTAGCAACCAATGGATATGCATTTATGGAACACCT
GCTAACTGCGACACACACTCCTATGTATGAGGGAAAATACAAAAAATGTTAAAGGAG
ATGCCTTCCCTTGCCCTCAGGAAACTTAAGTATAGTTGCAAAGAAATGATTAGCAGC
AAACGAAACCATGGAGAAGTAAGGGCTAAGGTCTGTGAAACAAGCCTAGAAAATAA
CCTTGTCCTTGAAAAACACAAAAAGAAAGAAAGAAAGAAAAGAAACTCCAAGGCCC
TTGTGAAGGAAACCATTAAGTTTGCTTCACTTCTGTGTTTAGGAAGACACAAACCCAG
TCTTAATGAACCTCAAGGCCACAACTACTGGAGACATTTAGGAATTGTCACCACATTC
TAATGTATATATCCTCTGTTTGGCCCTTCCTATTAATATTTTGTAAAATTTTTGAAGAT
ATGAGCAATGTTTAAAACCATGAATCCCCCTTTTTTTATAAGTAATATTTAGGCTGAA
TAAACAAGAGAAAATAGGACATAAAGGGGAGCCAACGTGTGCCTTCATTTATAATGT
ATTCCCAAGTTGTGAGTTTGGTTTATCAGCAATTTATCATGCCAAATTCCAAGTCATA
TTTATCTATGCAGATCAAACACTTGATTCTATTTTTGCCTTAATTTTTTTATTGGGTAT
GTTTATGACCAAGTCATATGGTATTTTCTGTGACAGATAAAATGCACAGGTTATTCCA
ATCTGGCTCAGCCAGTCATAGCAACATGTAGTCCTTCTCATGTCTTAAGAATGAGTAT
CAAGAATTCAAAGGGAGTTCCAGATGGCATCCAAAAAGCTTACAGTTTATGCATCAC
TTATTCTAACAGTAGAAAAAGAATATTTGAAGCCAAAAATAGACCTTGCATGTAGCA
TGTGGAAGAGTAGAAATTGCCCTGATAGTTAAACAATTTGAAATTCAAGACATTAAT
TTCTTTATGAAGCATTTGTCACATCATAGGTAATATTTTATGCCTATCATATATATACT
TATTATGAAATACAAAGAAATTATTCATTCTATCTAAGACTTTGTATCCTTTACCAAT
ATCTCTCCATTCTCCCACCTCCACCCTAGCCCCTGGAAACCACCCTTCTACTCTCTGCT
TCTATGAGTTCTTTTTTAGTGAGATCATGCAGTATTTGTCTTTCTGTTCCTGTCTTATTT
CACTTGACATAATGTCCTTCAGGCTTATCCATGTTGTCACAAATGACAGAATTTCCTT
CTTAAGGCTGAATAGTATTCCATTGTGTGTATGTAGCACATTTTCTTTATTAATTCATT
TGTTGATGGATACTCATATTGATTCCATATCTTGGGTCTTGTGAATAATGATGCAGTG
AACATAGGAGTGCAGATATCTTTTTGACATACTGATTCCACTTTGATGGGATATATAC
CCAGTAGTGGGACTGCTGGATCATCTAGTAGTTTTATTTTTTTTTATTTTTTATTTTTTT
TATTTTGAGACAGAGCCTTGCTATGTCGCCCAGGCTGGAGTACAGTGGTGCCATCTAG
GCTCACTGCAATCTCTGCCTCCTGGGTTCAAGCAATTTTCCTGCCTCAGCCTCCTGAG
TAGCTGGGATTACAGGCACGCACCACCATGCCCGGCTAATTTTTGTATGTTTAGTAGA
GACGGGGTTTCACCATGTCTCGAACTCCTGTCTTCAAGTGATCCGTCCACCTCAGACT
CCCAAAGTGCTGCGATTACAGGTGTGAGCCACCACGCCTGGCCTAGTAGTTCTGTTTT
TAATTTTTTGAGGAGCCTCCATACTGCTTTCCATAATGGCTCTAGGAATTTACATTCC
ACCAGCAGTGCACAAGGATTGCTTTTCTCCACATTCTGGCTAACCAGTCTCCTGTCTT
TTTGAGAACAGACATTTCAACACGTGTGAGATAATATCTCATTGTGGTTTTGATTTGC
ATTTCCCTGATGATTAGTGATCTTGTGCCTTTTTTCATATAACTGCTGGACATTAATAT
GCCTTCCTTTGAGAACTGTGTATACAGGAGAAAATAATCACTTCTCAGAGGAGCTTTC
ATTTCAAAATATCCGGGAAAAAAATAGAAAAAATGGAAAATTTATCCTAGAGTAAGT
TGTCTTTTATATTTTGACCCTGTTTGTGACATAAACTGGATGATACAAAACTGGAATG
CAAAGGCTTTAGGAGGATTACTTACTTACTTGTATATTGCTTTAGGTTGTTTGCAGAA
AATTATACTAATTGAAGTTCAGGCTATGATGTGATAAAATCTATGTCAGGAGATGAG
TCTACATGCAAAGTTTGAGGAAGTGACATTTGAGTTTCAAAACAAAAAAGCAATTTT
CAATGTCATATCTAGGTTAACCCAAAAGATTTCTTTCACCCTATTTAGCTGCCTCTAA
GATGGATGCTGAGGATAATTACACTGTAGAACAATAGGACGATGCTTCACACTCACC
TCACAGGCTCTGTTATTCCCACATACTGCCAGAGATACTCCAAAATAAAATCACTGCA
ACATCAGGCAGTTATAAACCTCAACGGTATTATTTTCTATTTATATACAGTATATTTT
ATATTTTACAAGTATAAAATAGAATATATTTATTCTATTCTCTTTGACACAAAGTGAC
CATAAGACATATTACTTAAGTATGACTAGCAAAGTCATGGGGCTTGTCATTCAGGAG
GAAACTCTTAACTAACTGTTCAGTTTTTGTTCACTGCACCATTTACATAAGCCAAACT
AATGCTTCACACTGTGCAAAACAATGCACAGTGTTGTGAATGAATGGCTAAAATAAA
ACTCTAATGAGTGGGGTTTGAAAAATGCAACTTTAGAAAACTGTTGAGAAAATGTTG
CACACTGCGCATTTTACAAAATTTCGTTGAAGGACACTGGATATTCTTTTTAGGATTA
TGGAGGGAAGCAAAATTTTGGCTCCTACATGCAGTTTTTGTGGCCTTTGCCTGAAATA
GTCATCTCCCATTAATTATTTAGATATCATTCATTTCCTAAGACAACATTTAGGGAGA
CTGCCTTAAGTACAATTTGTACACTACCCAGATAAGAATTCTTTTTGGTGAAACATCG
ATAAATATTACTTGGCAGTAACACCAAGTTAAAATATTTGTTTCACAGTCGACGTTAA
TAACTATTATAGATAAAGTGAATTTTATAAGACATACTCAGATCTAAAACAGCAATA
TGGAGCTCTTCAAATCCATTGAAACTTCATACCAGCCTACGGAAGTAGAGGTTTTTAT
GCAAACTCTTCAAGAAATATGCTCTGAACTTTTAATTCCTTAGATTGATAGAGGAATT
AAATCATGATATAACTAATAGGTTTGTGGTACAAATTGCTGCTGCTTAATCTGACTCT
GTGTCTTCCCAGTGTTCTATATGAATTAGATATTCCATTATCTAAAGACAATCAACCC
CATCCCACGGTGATAGCTCTAGGACTCCCTTTGAGTTCATTAAATCTGTATTCTCAGT
CTCCAAACTTCTGGTTAATTCAAACAGAAAAGTCAACTGGCCCATGAACTAAAATAA
AGTCATCTGAATTTTTTTTTTATTTTGCAGTGTGATAAAAGTCTCGCACTTTTTATTTC
TGAAAGTTTCTGCTTTCACTGAGAGCATAATAGGCTATCCACCCTTATGCAATCTTAC
ATACAAAGTCATAGTCAGGCTAAATTCAAAAACACATGTGAGATAGAAGTCAACGTT
TATTTTCTGGAGAAAAGCCACACATTACAACAAAGTGAACAATGAAGCTGGCATCCT
TATCACTGGTGACCAAAACATTTGTGACTCTGGACATTGGCCCCACAAATGCGATAA
ACATTCTGCATAGGAAGTGAGTTTTGCTAATTAAAAATGGATCCAAAATACTTTCTAC
TCTTCAGCCAAGAATTAAAAAGTAATAGGGAGGAATTGAAATCACTTGGGTGCTACA
TTGAGCCATTCTGGAGAAGCAATTCAGAGAATGTCATGGCAGCCTCAAATTGCTGCT
CAGGAGCATCCCAGCTTAGAAGATTGCAGGAAAGGAAGAGCAAAGTCATTCTTACAT
GAGAACTGTCCTTAACCAGATGAATAGACTCTCCATTTTTTACCCTGGCTTTGTCTCA
TTTAAGTCCCAACCAATCTAGCTATCATTTTAGGTTTTACTACCTGCTAGTATTTAGGA
GCTTAGGGGGATAAAAAAATCCCTCAATACTCAGAATTAGACTTGGTGATAAAAATC
TTGACACATAAACAGAATAAAGCGCTTTCATTACTCCTCTAAACCACAGTGTCATTTG
GTCTCTATCAAGGACTGTAAGAATTTCTTTCATCAGGGGAAAGAAAAAAAGGACAAG
AGCCTGCAAGATGTAGCGGAACTCTCATTAAACACAGCAGGAGCTTTAACTGGAATC
CAGAGTAAGGTGAGGTACCAGGTTACAACAATTTACTGCTTTTATTACAATTTTGATC
ACAAGGACTGATTCATGTCATCTAGTTTCTTTTCCTTGTCACTATCACTGGTGCTAAG
AATACATCAAATTGAAATTTAAGAGCCTCATATGTTTCTGTATAACCCAGTGATGGGT
TGTACTGCTTTGACCTTCTTAAATGTCCCTTTATTTCATTTGATATCCATTCCCATAGA
AAAACTATAATGCTTTGGTTGGTCAAAATATTAATCTTTCAAAACCTCCCTGGCTTAG
AAAACCAAATTTTTGTAGAGAGAGATGGGTAGAATCTAATTTTATTCTAAAGCAATT
AGCATTACATCATCACAGCAGAAATATCTAGAATATTACCTCATGTCAGTGATCTTCT
GATATGTTAAAAAGGGTATTTTAAAATCTGAGTTATTTCTTTTTCTTTTTAAAGTTACA
TCATTAATTACATACTCATCAACCAAAATATTTTATGCTCCAAATTTGAACCGATATA
GTATGTAAGAAGTGTTCAAAATGAAATTATTTTGGTCTATTTTGTCTTTGAAGAAGAT
CACAGGGATGGACCTCCCAAAAGGATTTTTAAATGGGATTACATATCTGACTTTTAA
AAAAAATTATCTGACCTTGAGTTATAGTGCCCCAAAGTAAGCAAAGTTCCAAACACA
CAGTATCATCAGAATTGAGTTAAAATTATCACCAGGGGCTTAATTTCTGAAATTAAA
AAGGAAATGTTATTTCCTTATGAAAAGAAAAGGAACCAAAAATGAACTTCAAGGTAG
CTGATTTCTGTCTATGTTAAGACTTAGGTAATGGGAGAAAGGGAAAAGGAAGGACAG
AATTAGGAGAGGAGCAGTGTTTAACAATTGCGGGTGCAAGACTCAAGTTTTTTAGAA
TCCATTAGCAGAGAACCCTATTTCTCCCATTAACTGCTGTCCTTTTAAATCCTGGCAC
CAGCTCTGAGGACTGCAGGGTCCATAGCTAGTGCCCCACTCTACCCAGTTTAAAGAC
ACCACTGCCTGGAAATGACAGGGGTTTTTTTCTTAAGGAAAGAGGTGCTTTCTGCCAC
GTATATATAAATTGGTAAGCTTCAAATAAAGTGCTTTTGTCCTTTCTGTCTATCAGAA
ACTGTGCAAATCGAATTGCTGTAAAACCAAGGGCAAGAGACATCAATCCTGCATTCT
ATAGCATCTGATTTTATCCTTTATCCCCAGGCACATTTCAAAAGGAAAAAAATGAGGT
TGCATTTAAATTGAGTATTTGGGACTTGCCAGGAAAACCTCCCGCTAGACTAATATGA
TTGCAGGGAAAACAAGAGAAAGGAAAAGTGGAGAGGGAGTGTGCTAACAGATCCTG
GGCCTCGTCAGCAGAGCCGTCCTGAGCACAAGGCCATGGTCAGACATCTGGTCCCGC
GAATGACGTTTTCTTTATGGTCATTAAGAACACCAGTGTGTCGGGACACAAACAAGT
ATTCCTTTCAGGGATTATGACACATTTTCTCCCAAAGTAGTATATTAATGACATTTCC
AGAGCATTCTTTACTATCTTTTATATGTGATCAGGAAGACTAATACATATCACTACTT
CTTTTACACACAGCATTAGCCAAAACTAAAGTGTCAAATACAATTTTGCCTAGGATG
AATAAACAGAAGAAATTTTTATGATACTGCACTATCAATTCCAAATTAAATAACAAC
AAAATGATAAGTGTTAAAATTCATATTAATGATTGTTCCCACACAAGCCGGAAAAAA
TCTTTCTAAGAAGTCTTTCATGAGTTAATCCCATCTTTCAAAGTGTTCAGTGGCTCCG
AATTCAGTTACTGTTTCCTATCAGTTCTTCTTTCATTAAGTCTCTTCCCTTTTTTTTCTC
TTTGCACTATTTCCCTTAGCCGGGTACATAATCTGCTGTGCTTTATTCATTTGTGTCTT
AAGTTTGTTTCCCGATGACATACCTTTCCAGCAACGCCATCTGGGGAGTTTGGGCAAC
TGTACCACGTTAGGAGGAAACCCTTCTTCACAGGAGAGTGTGCCTTTGCTGCAGGGA
AGGAATTAGGATTTGCTTGGACTGTGGTTGCAGCTGGCTTTTAAGGATCTCCTTAGAA
TGCAAGCAACTCATCAATGAGAATCTCTGCAATGGTTGTCACTGGGTAGAGTCATGC
TATGTGGGGTCATAGCCTTTGAAACAAATAACAGTAAAGATAAAAATGCTATTAAAG
GAATCACCACCCACAGAGGTTAACTGGGTTTTGTCCCCAGACCACCTCGAACAAGAA
AGAACATTTTTATCAGTCATTTTCTTAGTTTTAGCTGATAAAACAAAGTACCATAGAC
TAGGTGGCTTATAAACAACAGAAATTTATTTTTCACAGCTTTGGAAACTGGAAGTCTG
AGATCAGGCCGCCAGAATGATCAGATTCTAGTTAGGGCCTACTTTGCTTTTGCAGACT
GCCAACTTCTAGCTGCATTTTCATGTGGCAAAAGGAGATTGAGCTAGCTCTCTGGTCT
CTTCTTATAAGGACACTAATCCCATTCATGAAGGCTTCACCTTCATCATCTAATTACT
CTCCAAAGACCCCACCTCCAAATACTATCACATTGGGAATTAGATTTCAAATACAAA
TTTTGCGGGGACACAAATATTCAGTCCATAATAGTAATGATTACTCATTATACATAGG
GCTCTAAATGTGCTAGCTTCTGATAGTTTTTACACTCACTTCTCTTTATTAGCTTGTCA
AGCATAATTAGGGCAGTGGCCTTACTGAAAATTATTGAATTTAGTTTCCTAAGGACA
GATATTGAGGAGTTTTTTCTTCACTAAAAATTCACGTTCCGATACAGCTTTCATCTGTT
ACTACTTTGTGAGATGGAAAATCTTTTATTTTATTTTTATGTTTGGATTGACCCTTCTT
AATAAAGTCGGCATGTAATATGCTTCATGTGTTTCTAATATGTGCTTAATTTTGCAAA
ATGTTTTGCATACCAGAATGCATTTCTCTTCCAAAAAAGGTACCAGCCTACAAAACCT
TGCTGTTACTGTTTTCAATTAGTTCATGGAATTAAATGTATTAAATGTTTTATGCTCTG
GCAGAAATTATGATTCTCACTTAACTCCATATAAATCTGGATCTGCCTGGGCCTTTAT
AAGTGACACAATTTCATTAACTGAATAAACAAATGATACAAAGAAATTTGGTTTAGC
CTTCTAAAATTCCAAAGGCGTTCAACAAAATATCTCAGAATGGATGTTCCAGGACTTT
TATGGCACAGGACAACATGTATTGCTTATTTTAAGAAAATAAGCTAAATAGTGAGGG
GATTCTTTTAGCAGATCCTCAGGATGTGTTAGGTTGAATCATAGGCAAATGATATTTG
ATCATTGCACCTGTTAACACATTGAACCTCATCCTAAAATTGTAGAGCTAGAAGAAA
GCCTTCTGGCAGTTTTTAAATAGATTGATTTACTGCAATTTATCCAGAAGCTTCACCG
TTGTCACTGGCTACATGTGACTTTGGCCTCTGTGGGGCTATATCCTCATTTGTAAAATT
GGTGGTGAGGTAGGTGGACAGTTGACTAAATAATCTCTTAGAATAATTCTAGTATCT
GTGGATCTAAAGCATCCAGGGGTTGAATATGTTTCTTTCTGGCCAAGAAAAGATGCA
CCTGTCAATAATGCCCAAACTCATCTTCTGAGAATCCTCTTTCCCAAGATACCCACTC
TCCCTTGGGTTATATTATAGTAATGATCAGAAGCCCCTGCCAAGAAGAAACTGTTAA
CCTGGGAGGTCTATATTTTATTTCACAGCCATCTGTTTATACTTTCTCACAAGTTAGTG
CACAGTATACCCATCATTTTCTACCATTTTCCTTAATTTATTAATTTTACTAATTGCAT
AATTAACAAAAGTAAGAAGATTTTACCTCCTTATCCCCATCTGGTAGTTTGCAGATAC
TTGGCCTGATGACAACTGACAGTGATGAGATACTCACCAAGTTTACCAGGGCAGGAG
GCTTCCTAGAGAAAAAATGAGAAAATGAAATGGGGAAGGGGAGTGAAGGATTGAGG
AGGTGACAATCTGGACTCTTGCAACTGCATGGCAAGGTTGGCACACAAGCTGGGTTG
CAACGGAGGGAAGGAGATCCTTATCAGATGTAATCAGAGCTCAGATCGAGGGCTTTG
GTGTGTGTAGAAAGAGGGAGAGACAAAGAACTTAAAACAGAGCTGCCATTTGACCTT
GCAATCCCATTACTTGGTGTATACCCAAAGGAGAATAAATCATTCTATTAAAAAGAC
ACATGTGCTTGTATGTTCATGGCAGCACTATTCACAATAGCTAAGACATGGAATCAA
ACTAGGTGTCCATCTATGGCAGATTGGATAAAGAAAATGGGGTAAATATAAAGCATG
CAATACAACATGGCCATAAGAAAAAATGAAATCATGTCCTTTGCTGCAACATGGATG
CAGTTGGGACCCATAATCCTAAGTGAATTAACACAGGAACAGAAAACCAAATACAG
CATGTTCTCACTTATAAGTGGGAGCTAAACACTGAGCACACATGGACATAAATATGA
GAACAATAAACACTGTGGACTACTAGAGGGGGGAAGGAGAGAGGTTTGTAAAACTA
CCTATCAGGTGCTATGCTCAATACCTGGGTGATGGGATTTACACCCCAAACATCAGC
ATCATTTAATATTCCCATGTAAAAAGACTGCACATATACCCCTTGTATCTAAAATAAA
ACTTGAAATTAAAAAAAAAAGAAAGAAAGAAAGAGGCTGGAAATAGAGGCTCACAC
CTGTAATCCCAGCACTTTGGGTGGCCAAGGTGGGTGGATTGCTTGAGCCCGGGAATT
CAAGACCAGCCTGAGAAACCTGGTGAAACTCTGTCTGTACAAAAAATACAAAAATTA
TCCAGGCATGGTGGAGCGCACCTGTAGTCCCAGCTAATGGGGAGGCTGAGGGGGGA
ACATCACTTGAGCCCAGGAGGTGGAGGTTGCAGTGAGCTGGGATCACACCACTGCAC
TACAGCCTGGGTAACAGAGCAACTCTGTCTCAAAGAGAGAGAGGAAAGAAAAAAGA
AAAGATGGACAGATAAGAAAATGCACTTGGAGATTAAGAGAAAGCAGCAACATAGG
ACCCTGGATAATGTGTTTGCTTAATAACTATCCTGATGAGTTATCTGACTATTCCCAA
ATGAGTACGTGGCAATTCAGGCTGAACCATCAGAGTAGCCCTCCGGAATCTTACTTA
TGTACAATAGACCTGCATGCACATTTACTAGAATGAGCCTCTCTCTCTGGTAATCATG
TCTGCTTCCACTAATTCCATCTGTTTCCTCTCTCTCCCTCCTATCCTGCTAGATCTTAAT
TCCTTCGACCTTCCTTTGTTTTTCTAACTCCCTTTCTTTCTCTTGTTATTTAACCTGCTA
TACTATGCAATTGATCTCCTCTGCACTAAGGAACATGCACTTCAGAATTCTGTTGACA
TCTTGCATTCCTTTATATTTAGTGAAAGAATGCAAAGGAGTCTACCTGGCAATATTCA
CTCTGCAGGAGGCAATAATTATTATTCAAATTAAAGGAAGCAGTAAAGAGAAATTCA
GAAAAAATGAAATATACTAATCTTCAGCTTTTCATTTCAGCCTACAAGGAAAAAATG
AAGGAGCTGTCCATGCTGTCACTGATCTGCTCTTGCTTTTACCCGGAACCTCGCAACA
TCAACATCTATACTTACGATGGTGAGTAACCTAGGATAGACATACCCCTGCTAGCTA
GATCATTTGGAAAGGTTGACATATATTTGTTTCTTACAGCTCCTGATATAATTACATC
AATATTTTGTAGCTCTCACTATTGACTTGCCGTGTCTAGCTATTATGTCCAATTGATTA
CCTATTGCTGAAAACAGTTTGAATTTGGTGCTAATAACAACACATCAATGTCTGTTAA
GAAATGTGGATGGATTCTTATTAACAGCCACATCCAGCATATCAACATCCACAATAT
GTCTAAGGTCTTTCTTTGCAAATAATTTAATAGGCTAAGCCATAATTGGAGTAGATCA
TAATTTGTAAGAAAATGCTTTATACTTAGAAAACTCAAGAGAAAGAATCAACAACCA
TAATTGTTTTTGCTTTATTGTAGTCTTTATAAAGTTTCTATACTTTGTATATACATGTC
AACCAGCTAATGATAATAATAATTGGCTCAATAAATAAAACTGACTTACGACTGAGG
CCCTAGATAAAGAGGGTCTGAAAAGAAAAGCCTAAAGAATTAGCATGGCAATTAAC
ATGATTGAGGTGCAACTCTTTAGGTTTGATTTATCCTGATTCATTTTGCTTACTTTGGC
TCTGCCACAATCCACATGATCTTGGTCAAATAGATACTTGGATTCTCTAAGTCTCATT
TAACTCTAGCATCTTCCTCTTGGAGTTGTTGTGAGGTTTAAACGGTTTAATGTAAGTC
AAATATGCAAAACCAAGCCTAGCTCATTATATCACTCTACAATGATAGCTATCATTAT
CAACATCATCCTTACCTAATTCAGTCAATTTAACTAAAATATTTTATACAGTTCTATGT
ATCCTAGATATCCCTAAGGCATATTTTACTAACTCTCAGGCTCACAAATATTTTTCTTT
TCCATATATGTAAAGAAAGACATTAATGACAAAACAAACTGACCTTGTGGCAGTTAA
CCCCTTCTGCACCTTTAAAGCCTATTCAAGGACTCAAAGGCATTTACCTTCCAAAGTT
ATTCTATCGTAGCACAAAAATCATAAATGCTAATTAACTGTTCCATAAGGAAATGTCC
TCCATGTGAAAGGAATTCTGTCTCCAAACAAAACATTCATTAGAATGCAGGGCCAAT
GCCTACTTTGTACAAATTCATTCGGTCAGCAAATAAATTAGACAGACCTTTATTATTT
GCTAGATGTAGCTGTGAAGAAGGATCCAGCTATGTTTCTTATGAGACTAATGTCGAA
CTATGGGTTGTCACTGAGGATCCAGAGTTCCATAGGGCGTAGTCCTCACCTTCAAAG
AATTCAGGGCTTAGTAGAAGAGTCTTACACAAATGACTAGAATGTAGAACACAGAGT
GGTTAGGACAAAGGAGCCAGGGATGGTTTTTGCTGGGTTAGGGAATGAAAAAAGGG
GAAGAAAATATGTGAAGTTATGTGTGAGCTGATTCTTGAAATAAGCTGTTTTTATTTG
CCTGCGTTCTCTTATAATCCTTTTCCATAGGCTTCCATAATTTTTATTGAGCTGTATTT
AAAGTTGAATAGATAATTCAACATTTCTCGTAAACTGTGCTTCCTAAAAGAGTCCGTA
GAGAATTTCAAATTTCTGCAGTCTTTAACTTGACCTGGTATTTCTATGTTAGATAATA
ACGTGACTTGTTTATTGCAGGCAAACATTATAACAATAAATTATTATTATTGTTTACA
TTTGTAAGCACTAAGTATATGGCTTGTGCTTTGCATTCAGCATCCTTTATCATTTAATC
TTCACAACCACCTTAGAAGGAAGGTACTCTTTTTATTTCCATCTTTTAAATGAGGAAA
TAAAAGCATAAAGAAGTTAATTAACTTACCTAGTGTCACACAGCTATTAAGAGGGGC
TTACTATTTGGATGCAAATATAGGCAGTTCTAATTCCAGAGCCTCTAATCTAAGGCAT
TTAAAACCCCATCACCTTATCAAATAAGCTGTTTTTATTTGCCCGTGTTCTCTTATAAT
CCTTATCCATAGGTTTCCATAATTTTTATAAAATTGTATTTAAAATTTAAGTATAATCT
TGGATGCCATCAGGAAAATGAAAAACATTTTTACATTTGTGAAGGAAAAAGCCCACA
TCATTTCCAATATAGTTATTGAGTTAGTATTATCTAGACTATCTATTAGCAGCTAAGG
ATCTGAGGTCAAGGCCTGCCAGCCTGGCATTTTACTTGACCACAACCTCCATGTGCAC
TAACCAGGCTGCTAAAAGAACATTAACGGGAACATAACCTGCTGGCTTGGTTGCCAC
AATTTTAAAAAGACGTTAATAAATTAGAGAGCACTTAGAGGTTAGGAAATAATATGG
TGGTAAAGATCTAGAAACAGTGTCATTCTGGGGCACTTGAAGATGTTTAGCCTGGGG
GAACAACTTGAAATGGAACATAACTGTTTTCAAATACTTGAAAAATGGTGGTGCACC
ACAGAGAATGGCCTAATCATGGGTAGCTTCAGACTTCAAACAAGGATCAGTGGGCTA
AAACCAGAGAGATGGAGTTTGGGACTCAAAGAATGCTCATCTGAAATTGAGGGCTGA
CCAGCGAGGTTCTTTTAAAAATCATTGCATTTTACTAAATTGTGAGTTCTGTAATTAT
AAATGTCCTAGCAGGTGCTAGCTGTCATCTTTTCTATTATAAATTATACTATTTTATGT
TATAATTTGTATTATACAGGCTTAAAACATAAGGGTCTGATAATCTGCTTATCTTTAA
TACATAAGCCACTGATAGAAAATAAGTGGCTAACCATTCTTCAGTTCTTTTTTTAATT
GACAAAAATTGTATATGTTTGCGGTGTATGGCATATTTTGAAATATGTATACATTAGA
GAATGGCTAAGTGAAGCAAATTCACATATGCATTACCTCACACACCTGTCATTTATTT
GTGATGAGAACAAAAAATCTACTCTTTCAGTGATTTTCAAGAATACAGTACATTGTTA
TTAACAATAGTCAGCATGGTGTACAATAAGTCTTCTGCGGCCGGGCGTGGTGGCTCA
CGCCTATAATCCCAGCACTTTGGGAGGCCAAGGCTGGCAGATCACGAGGTCAGGAGT
TCGAGACCAGCCTGACCAACATGCTGAAACCTTGCCTCTACTAAAAATAGAAAAATT
AGCTGAGTGTGGTGGTAAGCGCCTGTAGTCCCAGCTACTCAGGAGGCTGAGGCAGGA
GAATTGCTTGAACCTGGGAGGCGGAGGTTGCAGTGAGTCGAGATAGTGCCACTGCAC
TCCAGCCTGGCAAAAGAGGGAAACTCCGTCTCAATAATAAGTCTCTTGCATTTGTTCT
TCCTGTTTAACTGAAATTATGTATTCTTTGATCAACATCTCCCCAGTCTCCACCCCTAA
CCCCTGGTAACCACAATTCTACTCTGCTTCCGTGAGTTCAACTTTATGAATAGTCCAC
ATGTAAGTGAGATCATGTGGTATTTGTCTTTCTGTGCCTAGCTTATTTCACTTAGCATA
GTGTCCTCCAGGTTCACCCATGTTGTCAAAAATGACAGGATTTCCCCCAACTTTTTTA
AGGCTGAACAGTATTCCATGTGTATGTGTATAAATTAGATTAGTAGATGTTGCCACTC
CCTCCTCCACCACAGTGGCTCTATCCCTGGCTCCTGGCTCCAGCCGAGTACACTAGAG
GAGGATATTCTAAACAGCAACAACACAGGAGCAAAGACATTACAATGGGGTGTTGTC
TTATTGCCCCCATTAGACTGTAAGCATCTTGAAGACAAGGACCCCCATCACAGAGTG
ATGTTGTCATCCCTGGAGTGGGCACTGTGCATGATTGATGACTGGAAGCAATGAACA
TACAGAAGGGCAAAACAGAAATCAGCAGGATGCTTTGCATTTCAGCATTGACTTTGC
CAAATATGCCCAACTGTTCAGGGAGTTACATTGGTTCTAACGAAGCTCCTGTGATTCC
TAAGCACAGGAATGGTGATAATATATATAATGGTGCATGCATATATACGCATATCTA
GATAATGATATCTCATTATATGTGAGAACTGAAGAACTCCGTTATGTTTCTCGTCTAA
CCAAAAAGGGCCTACAGCTACGATAATTTCCAAACAAATAAATCTGTGCTACTTGAT
TTTCATGCAAAGCTCATATTTGTTCAAAAGGAAAATAAAGCTTAATTTAAAATCAATT
TAGGCTATTTTTATCTAAGTATGCTTACCGTTATTCAACTCCCTGCAGATATTGTCAAA
TTTCTCAATATGGTAAATATTTATTCTGTTAAAATATATCCATAGTTACACTAAAGAC
AGAGAGGTCTTATATGTTCTAAACAACATAGAGCAAATGCTCATAAACAGCATTTTA
TTCCTATCTCCCGGAATAACAACGCTACTTCCAATTGCTGGAATCTAAATTATTAAAA
TAAACCCATGCTGCAAGCTTTGTATGCTTAACATTCTCAAATGTTCACTTTTCAGATA
TGGAAGTGAAGCAAATCAACAAACGTGCCTCTGGCCAGGCTTTTGAGCTGATCTTGA
AGCCACCATCTCCTATCTCAGAAGCCCCACGAACTTTAGCTTCTCCAAAGAAGAAAG
ACCTGTCCCTGGAGGAGATCCAGAAGAAACTGGAGGCTGCAGAGGAAAGAAGAAAG
GTAACTTTTTCCATAGGTTTTCCTTCTCTCTCTCCCTCCCCTGCTCCTCCCTCTCACACA
CTCGGGCACACATGCACGCACACACACACACACACACACACACACACACACACACA
CACACATACAGAGAGCAATGACAGCTGAACCTGTGCCATGCCAACATGTATAGGTTT
TCAGTAGACACAGAGCCAGGCTAGTTGGGGTAAAAACTGTAAGATAGATGCTAATTT
TAGGCTAGCCAAACCAGAGCTCTCAGAAATCCAAAGAGCTTCAGTGCTCTAGTGCCC
CTTCCCGTATATTGAATCCCCTTATTATAAAAGCCTCCCTTCCCTAGACCATCAGGCA
GAAGCACTGTAGAGAAAACACAGCCCTGGCGAACTCCAGTGGTGGGGAGGGGAAGA
AGTGCTGCTTCCTCCCTCTCAGGATCTGTGTCACCCCCTTTGTCAGGCGTGGTTTTCCT
TGGAATTACAAATTACCAGATCTTCCCTCCAAGATCTTTCCTGCCCAGGGTAAGGGCC
AAGAGCTTGCCCCTTTCCTCTTCAGAGTCCCACTGCCTGCCCTGGAAGTTGGTCCTTC
CAAGATCAGGACCTTCTCTGAGTTCTTTGAATATGTTCTTTATCTTTTTCTAAGACTTG
ATGGGGATTTTTCTCTTTTTGCCATTGGTCCCTGCTTATATTAAAGAGCTTTCCTTTTG
CCAAATCTTTACTTTTCCATAATCACATGGCTAAGAAGAGCCAAGGGTATTATTTGAG
AACACTTAGAAATCCTAGGGACTGTGTACACAAACAGAAGTTGTTTGAATGTGTCTG
TTCCAACCATGTGGTTATGGTAGTTAATCCCATCAAGGTACTCACGATCATCCAAAAA
TGGAATTCTTTTATGTAATTCATCCCCACATTGTATTTCCCAATATTTTTTATGATATA
ATTTTAGAATCAGGTAATCACTAAGAACATGTTCCCTGCACAGTTTTATGATGTTTTC
TCTAAAAAGTCAGCCAAAACTTTGGACACTTCTATGTTGGATAATTAAAAACAGAAT
GAAGATAATCCTCCTCCTAAAGATTGAATTCTCCAAGAGAGAATGCAGGACAAACAC
AGATGTGCTGTGTATAGTATATGTGCATATATACATGCATATATGTACACAAATATGT
GTATTATCAAATAATGAGGCTCAAACATTAGAAATCCTTAGATTAAATTTTCTAAACA
AGAAAACACTAATCTTTGTAGTTGAAAAAAAATCCTCCTATGATATGTAATATGCTG
ATCTCAATTTTCACCTAAGAGTGATGTTCTCCAAATGTCCGATGAGCATGTCATATAT
ATATATATGAATTTTTATATATATAATTACAATGGTAATTGGTATATAGAGATATCTA
TATTATAGATATATATAGCTATCTCTATATATTACATATACCAATTATAGATATAAAT
ATAACAATGGTAACTGGTGTATATGTGATGTGTATATATGTATATGTATACCATAATT
ATATATTAATATTGTATATATGCCATAATTATATATTAATATTGGTATATATACACCA
TGATTATATATTAATATTGGTGTGTGTATGTGTGTGTGTATATATATATATATATATAA
AATACTAGTTATCATTGTTCTAGATTTAAAAAACAGGAACCTGAGCTACTAACTCGAC
TATATATATATATATATATACAGGAAGTTGCTTTAAAACATTTTTATCAGCTTTTTTAT
TGTTATTTTTAGCTTTATTCTCATAGTAAAGCTAAAATAAATTATTCAACATTATCAA
AACTTTGCTGCCAGCAGATGTAAGCAATACCTAAAACAGTGGAGAGCATGTTGCACC
CAAAGCAGTTTAAGCTCTGACCCAAGCACTGGCATCTTATAGGCACTGGGTAGAGAT
AAGAGTCATAGGTCGACATATATTGAGATGCTATGACTTGATTAGAATATGGAGTCA
GTGACTGAGGTGAAATTAAAACTCAAACCACAATTCAACATCCTGATTTAGGATGTT
GCTGGTGTTTCTAGGTACTACACTTAATTTGAAAGAAATTATTGAGGATAAAAAAAG
AACTGGGATCAACAAAATTAACTAGGTGTTCTTATAAGAGTCCCTGAGGTTACTAATT
AATGAAACTGATAAAGCTCCTGCACCCTGACAGCAAGAAATTATCAATGATTATACA
TTTAAACAATTGAATTGAACTAGAAACTGGCCACATGGTTAAAAGACATTTACAAAT
GTAATCATCCAGTGTTATGATGCCCAGAAAAAAAAAATTCCTTAGAATGCTTTAAAA
GCCGTATTCCATCACCTTTCCAGTTATTTGTTAAACATTTTGTAATGCAAAAATAACC
ATATAGATTATGCCCTAGTGGTCGGGTTTTATTTTTAGTTTTTTATGGTTTTTTTTTGTT
AATGGTAGAGTTTTAATTAAAAGAAAATACAACTAATTAGCAGAAAGTGCCAACTTT
AAAAAATCACTAATTGATTTTATTCTATTGGGTTATACTGACTTAATTAGCACTAATT
TAAAGAACTATTAATTATCTTTAAAGAGTCTTTAGCAAGTGCATATATCTCAGTAATT
ATGTTAGTAAGGACATGCCTATAACCAAAACCCAACTCAACTAGTTAAAACAAAAAG
CAAATATGTGACTAAAAAGTCTAGGAGTGGCTACAGCATCAGGAACAGCTGGATCCA
GGGATCACAGTATTATCAGAAAACTTTCTTTCAGTGCCTGTCATCTCTTCCTGCATTTA
ACTGGTTTCATTATCAAGAAAGTTTAATTTCAATAGTCAGTTCCAAATTATTTTTCTCA
CAACTTAGCAACTCCAGCAGAAACAGAGCTTCTTTTTCCCAATAGTTTAACAAAAGTC
CCGAAATTGAGTCTCAATGGCCTGGCCTGGATCACAGGCCCAACCCAGAACCAATCA
TTATGGCCAAGAGGATGTAGTAGTTTGATATGCTAGCCTGAATCACATGCCCACCACT
GACCTGCAAAGGATTTTAGGTAAGATCCCTGGGGTAAGAATTGTGGAGGGGTAGTTC
CCCAGAAGAAAATCGAGGTGTTCTCACAAGAGGAAGGGGTAATGGATCTTAAATAA
ACAAAACTATAGATGTCCACATTTTCTATCTATAAATGTTTAGTGTTACTATAACAAT
TAGAATAATTATTTAGTTCATACACTATTCAATTTGTATCTCCCTTCTGTTGCCCTGTT
GCCGTTATTTTCTTACAGATAGAATGAAAAATATTAATCTAGGCAGCTCTGTGAAACA
GTACTGTCCAAGGAATATAACGTGAGCCAGGCCGGGTGTGGTGGCTCATGGCTATAA
TCCCAGCACTTTGGGACGCCGAGGCAGGTGGATCACCTGAGATCAGGAGTTCAAGAC
CAGCCTGGCCAACATGGCAAAACCCCATCTCTACTAAAAATACAAAAATTCGCAGGG
CATAGTGGCGAGTGCCTGTAATCCCAGCTACTGGGGAGGCTGAGGCAGAAGAATTGC
TTGAACCCAGGAGGTGGAGGTTGCAGTGAACCAAGATGGTACCATTGCACTCCAGCC
TGGATGACAGAGCAAGACTCCATCTCAAAAAAAAAAAAGAAAGAAATGTAATGGGA
GCCATATGTGTATTTTTAAATGTTCTAGAAGCCACATTTTTTAAAATAAAAGAAATAT
GAAATGAATTTTAGTAAAATATTCTTCACCCAATATATTCAAAACATTATTTCAATAT
GCATGTAATCAATATAGAAGTATTAATGAGCTGTTTCACATTATTTTATTCATACTAA
GTGTTTGAAATCCAGTGTGTATTTTACGTTTACAACTCATTTCAATTCATGTTAGACAT
ATTCCTAGTGCCTAGTAGCCAAAGGCAGCCAGTAGCACAGATACGGATATTAAAACA
GAAAACACCTAGTGAATAATGGGGAAATTTTAGGCCTAAGTTTTTAAAATCCATACC
AGATAATTATTCAGATTCAAATTTACTTTGTTTTTTCATATATATTCTTTAAAAATTAC
ATTAATATGGGAACTCAGAAAGTTCAAAAGAAATTTCCATTCTATGGTTTTAGTCTTT
ACATTGTCAGAACTAATGCAAGTGTGAAGTTTAGGATGTACTGTAAGTAATAGGATC
TTCTAAATCTCATGCCTTCTTCAGCTACCTACTCTGTTTCTATTTCAGTTCCTCACTGT
GGGGAGGGGACTTCTCTGAACCTAGGTTTCATCTCTCACTCTCGTTCATGGTAAACAG
GTTTTCCTTTGTGGCACCTAGCACAATTAGTAAGTAATTAGTATTTACTGGCATATTA
GTATATATATGCATATGTATTTATTTAACCCTATGTCTTCTACTAGATTATAAACTCCA
TGAAGATAGAACTTGTCTTTTGTTTAATAGTGCTTGGCAATAGTTATTACTGTAAACA
TTTTTTTTCTTTCTTATTCAACTCCTGTTAGTCATTGCCTGAGTACTACAAATGTTTTTA
AGTAAATTAATAAATAATAACTTTCAGGGCCAAATGTGAAAGCGGCAATATATAGCT
TGTTTTGATTTTTTATTCCACCCTCCCATCCTAAAACAATTATAGTCACTAAGTTTCCA
AATGACATCTGAAATTGCACTAAGGAAATCCTAGTCTGGGCAAAATCACTCAGTCAA
CAGATATTTATCAAGCACTTACTATTTGGCAGGCCCTGTTCTAGACACAGGGGATACT
CATCAAACTTACATTCCAGTGGGGGAGAAAGAGCTAATAAATACATACACAGCATAT
TAGATGATGCAAAATTAGCAGGACAAAGAGAACTGGGGGTGTGGGGGTGAAAGAAG
CTAATATTATATGTTATTATTACTATATATAATAATATAATTATTGGATAGTCAAAAA
AAAACCTCTTGAATAAGACATTTGAAAAGAAGCACAAAGGTAGCAAGGGAGTAGGG
CGGGCAGCTCTTCTCTGGGACCTGAACATTCAAAATGATGAGAGCAGCAGGTGCGGA
GGCCCTGAAATAGGAATGTATGAGGTGTGTTTGAGAAATAACATGGAGGCCAGCGTG
GCTGAAGCTGAGAGCAGGGGGAGAGTGGTAGCAACTGAAGTCAGAGGTCACAATTA
AGGACTTTGACTTCACATGAAATGGGAGATCATGAAGGATAATAAAGCCATTTCACT
ACTTTATGTGAATCACAGCATCTTTTTAAAGAAGTATCCTTTTTTAAAGGGGGAGATG
ACTAGAAAAATAAATAGTGTTAGATAAATAGAGAAAACAGGAAAACATTCTAGACT
AAGACAGTGATTCCAGAACTAAGGATCCACAGAGGCGAGAATGCAGAAAGTGTAGG
TTTCAGAGCAGTGGGTAGACTAAGGGTTTGGACTAGTGGATTTGGATAGGGAGTTGG
AGAGTAGCGAGGTGGGATTAGGGAGGGCTGTGAATGCCAGGTTAGTGTGCAAACTCC
ATTATATAAGCAGTAAGGAGTCACTACAGACTTTTCAAAAATACATACATGTTCCAC
CTGGCCCACGGGTTAGCAACATTTTCGTTGCCCTGGACCCATTTCCTTCCCAATAAGT
TACAGGTTTGTGAAGATTCTACCTAGCAAACATATTACTTTTAAATAACTATTAATAA
ATTATCTTACCATGATTATAATCAAAGGAATCTGTAATTGCTAATTATTTCTGATTATT
AAAAGATAAGCAGTATTGCACTAAATTGACATAATTCTAACTCAAAGTAAATATACA
GATAGACATGGCTATAGATGTGAAATATGATTTCTGTTAGGGCTTTTTAAATTTAAAA
AAACTTACGAGTTCTCCTCCCTCCCCCTACCCTTAATACCTTGAAGGCCTCTTTGTGG
GACTTCAGGGACCCCTTCAGGGAACTATGACCTAGGCTGTATTTGGGGGGCTTTCTGG
GTTTATAGCTGGAAGGCTGCCACAGAGGCATCGCCACTTGGGCTCAGATTCACTTTGT
GTTCAATGTTTTGGCAATGTCCCCACCTCCCCATTCCATCTGTTGACACTATTGCAGC
ACTGACCATCTGGTTACTAGGTTGGAGGATACTCCCTCGGGCTCCTTTGAACCAGAAT
TAGTGCTCCAGTGATTAGATAATAGAAGAAGCTTGTCATAAAAAGAATAAGCCCTTT
CCCTGCTTTTTCTCCATTCTTTGATTATCGCTGGTAGTCAGTGATGATCATCTCTATGA
GTCTATATCAATCTCATCAGGTCAGTTTGAACCTCATCTCTTGAAATCAAAGTTTCCA
TAATGCAACTGACCCACAAGGGTGAAATGACATGAATGCTTTAACCATCCATTTATC
ATTTATTCATTCATTCAACCAACATGTATTTAGCAAGAGGCAGCAGAGTTAGCATAAC
TATACATCCCAGTTGGCCCAGGACAACTCCAGCTAACTCTCGTTGTTTTGATACCATT
ATTAATTATTTCTCTTTACTCTCATAAGTGTTCCACTTTGGACAATCAATTACATGAGC
ATCCTTAGCAGGGCACAGTGTTTAAGGGCATCTTTAAAATATTGTCTTTAAGAACATG
TGGTTAAGAGAATGTCTGTGTTCAAATCCTGGTTCCACCACTTAAAAGCTGTGTGACC
TCAAGCAAGTGACTTAATCTCCGTATGTCCTCCTTTGTCAATCTGTAAAATGAGACTA
GTAATAGAACTTATGGAGTTAGTGTGAGAATTGGAAGGTTACTCTACAATAAAGACA
TATAACCAGCATGGTAAAAGGGTTAGCAATTACTATGTGAAGAAGCATCCAGTTTCT
GACCTCACAGAGATTATCTAGCAAACTCATGATTTTATAAAGAAAAGAAGTTTCTCA
TCAACAGAGACTGAAATGCTACCATACAATATACGTTGCTTTTTTTTTTTTTTTTTTTT
TGAGACGGAGTCTCGCTCTGCCACTCAGGCTCAGGCTGGAGTGCAGTGTTGCCACCTT
GGCTAATTGCAACCTCCACCTCCCAGGTTCAAGCAATTCTCCTGCCTCAGTCTCCCAA
GTAGCTGGGATTATAGGCACCCACCACCACACCCAGCTAATTTTTATATTTTTAGTAG
AGACAAGGTTTTGTCATGTTGGCCAGACTGGTCTCAAACTCCTGACCTCAGGTGATCC
ACCCACCTCAGCCTTCCGAAGTGCTGGCATTACAGGCATGAGCCACCATGCCCGGCC
AATATTTTTAAATATTATAAAATATTCTTTATCAAATTGCATAGAAGAAAAGACAGTT
TGATAGGTAATAGATATATAAATAGGTCAGGCCAACTAAAAGTGTCCTGAAAAAATT
AATATTGTGAAAACAAAAGGATTTTAATGACATTGATAAAATCTCACCCTAAAAGAG
ATTAAATTAAAAATCACCCTACTTGAACCAGTTCAGTGAGATTTCATTAGCATGCTCT
CATTACTGGCATAATCAGCTTCAAAGTCACTAAGCCTCTGAAAGGAAGATGTGTTGC
TTATTCTTAATAAAATGGCATAAAAGTAGATCATTAGTCACCAAACATGATAGACTT
ACCTTTTCCATTTGTTGGCATCTCACATTGTAGATGGCAATTAAAATGGAATCCAGGG
AAAGAGGGGGTGGTTTGTATAGCAATGGATTATGAAACAAAGTACTGGATTATTCAC
CGCTTGACATTCAGGAAACATTCTGCTCCTTACAGAATATGGCACGTGGGCCACAGA
ATCTTCCGTGTGCTACCTTCTCGGTGAAGAAGAGCACCCCCAAGTTTCTTTTCCTAGG
AGCTAACCACAGTAAACCCATTACACACTTTAGCAGAAGGGCTCATTCTAAAGGTCT
TAGGATTTTAATCATTTTAAATTTCCTGTTATGCTTCAGGCTCTTCAACACAAAGTGA
ATATTGTACTCTTTGGTTTTACATAATTATATTCAATTGTCATATTTCAACAGGACATT
ATTTGTGACTTTAGATGGGTCAATAATGATTTTCATTGTCAGCAGTAAAGTCAATAAT
TACAGACACATCACCTACCCTACTTGTGTAAAAGCATTTTTTGGTACTAGGAGATTTA
GTGTCTGATCAACGGTCCTGGATAGCAAGTAATATATCCCCCAAATAATGAAAAGTG
ACAAGAAAATAAATATGTTTACTTCAGAAATAAATGGAAAATTAGTGCTATCTAAAA
TGTAGTCTTAAGTCTCATCTGTGTACATAAAGTAAAATGAGTTTTATGTACTAGTTAC
TCAAATTTATCTTCCACTCCATTTGTATAGTAATTAAACTCTTACACTCAGTAATATAC
AAATTGGTAATTAACCTCTTTGCAAAATGTTAAAGTGTTCCTAAATGTACAATAAGTC
TCCTTTCCTGTCTCATTGTTTTTCGCTTCACGTACCTCTCATGTAATTATTTCAATGATT
GAGTTCAGTGTGAGGAGGTTTATGCCTAGAAAAGGTGCTCACCAATAACGTGCCTCA
GTTCCCATAATAGCAAGATCGAGAAGGTTCTTTAGTCTCCCGGAACGTCACGTTGAA
CATCTCAGTTCTATATTTTGCCTTGACATTTGCATTATATCAGCTGATCATTGTCTTGC
CCTAATTTTCCCTTTTAATATTTTAGTGACCTTCTATGTTAGGTACAGGTTATTTAGAA
GTGTTCCTCCAAGGCCAGATACTTTTTCCTTGAACAATTTATTTTTAACAACTTTTAGC
GATTTTCTCACTTCACCACCCTCCGTTTCATAAGTCCACGCAATCACAATTCCTTTCTG
CTAATCTGCACAGTCAAGATATAAAGTAAGAATACCTATTTGAACATGTAGTGAGAA
CTTTACTTCTCTGCCAAAAATGAAGGAAAATGCTGCCACTTTTGTATGTCACATGTTT
TTTATTCTACAGCCTCACTCACTTCATGTCATGTTTTAGTGCAGTTTTCTGGACTAACT
GCTTATTTTCTCATTGATTAAACTGCCTATTTGCTCATTGGAATTAGAGCCAATTTTTT
TCCTTGAGGGTCTGACTAGAAGATTAAACTATGTTCATGTGAGAATCAATTTCTACCT
AAGAAATGAGTTAGAGGAGTTATGGGCAGCAATATCTATCTGGATGCTACACTGTGA
AAAAGGAAGCGAGGTTATGCCTTTCTACCCCAATGGGGTAGCAGAGACCTCAGGAAC
TGAGGTAGATGCCCCCCTGGTTATTAGCGCCCCTGAATAATTTGTTCAAAAATTGACT
GCTGGACAGGTGTCGTGTTGCACGCCTGTAGTCCCAGCTGTGCAGGAGGCTGAGGCA
AGAGGATCTCTTGAGCCCAGGAATTTGAGGCTATAGTAAACTAAGGTCACACCACTA
TACTCCAGCCTGAGCAACAAAGCAAGACCCTGTCTCTAAATTTAAAAAAAAATATTG
AATGCTTATGAATAGAGACTAATATAGGAAGTCATAAGTATTTCCTTGGGATAGAAT
GCTTTCCACCATAATTGACTTGACATCCTGTATTTTTGTATGTGTGGACTTAAGTTTTA
AATATTTGAAACACAGACAATTATTAAGTCCTGCAAATGTGTGAGTTAATAGTGGAT
ATAACATTCCCTTCCAGGGTGTAAGAAAAGGTACCACAGAAGTGAGCAGCCCTGAAG
CACAGCCTGGCCTAGTTTGGCAGGTCTCTGTGAGTTAGCAGCAGACTCACGTGACCA
CACTCTGTACTGCCTTCTGTTTCTGTTTCACCCCATTAATTGTGCTAAAGAAATGCACT
TGACACCTATGCTGTGTAATCTCATTTAGCCCCAATAGCAACAAAAGTACTAACCCCA
TTAAATTGAGTCATTTCAAACTGAGCCAAATGTTGCACTCCAGTAAATGGAGTAGGC
ATTGGTTATAATGGGAATTCTCCATTATTCATAATGGAAACCACAGGAGTTTGTTCAT
GCAGATCAAATGTGTCCCACCAAGGCAAGAAGTATGGAAAAGTGGTGTTGCTGTATT
ACCTTGTAATTTCAAAGCCTTCCCGTCTGAATCTTATTTCCCTGCTGTTTCCTCTTGAC
TTTGGTTCTTTCACAAAGGAAAATTAAGAACACAAATATAAACATTAAGTTAAAACA
CAACTGAACAAAGTGCCAAACTTAATTGGAGCATCTGAAAATGAAACATTAGGCAGT
TGCAGTGGCCTCTTGATAATAATTCACAGTAACTCTCTGTAAGCTGATCCTGTCTGAA
GAGCAGCAGGCACAAGGCCCCTGGCCATGAAGTCCATCTCAAAGGGCCAGGCTCAG
CAAAGCAGGATGCAAACCCAGGCTTTCCAAATACCAGGTTGGGGCTCATGTCACTGT
GCCACAGGAGCTTCTGTAGAAAGGCTACTTGAAAAAAGTGGCCATTAAAAATCCAGG
TGGATCCTATCTAGGGCAGTGTTGGAAACACTGATCTATGGGAGGAGGAGCAGGAAG
GAATTGTTTAACCACTGAGCAGAAATGTTACATTGCTACCTGCCTTTAGCAGCTGTGG
CTGATGGGTACCAGTTGCTAAGAAGAGCATTACCTAACAGTGTATTAAGATAGAAAA
ATGATTTTAAAGCACGGCACTTAGAGAATGTTGAAGTTTTACTTTGCTTTATTTTGATT
TGTTTGGTTTGACTTTGTCTCCTGGAGCATCCTCCATGGATTTCTGTTCATTACAAGAG
AAACCTAGGGCTCTAACCCAATTCCTAATTCTTGGACACATTGCACCCTTGTTTTGTG
ATAATCCAGCCTTCTTCCTTGAGAAGGTTTGCTGGACTGGAGGTTACATGTATTGAAT
TTTCTAAAATGAAGGTGCAAAGCTGTCTCCTCTTATTTCTTTGTGGTGCTCACTTCACT
GTGAGATTTCCTATCAATACAGCCCAAGTCAGTGGGCATGCATGAGGTGGAGATGAG
GGAGTTAGGAAGGACTTGGACTCTCATCAACCATCAGGATCCCTGAATCCACTAACT
GTTCATAATCAAAGAAGTTTGAACAAATACTTCACACACATGAAATTGCCAAAATTT
TGCATTTGAGTTGTTATACCAGTAAGTCCAGTTGCCATCATCTCCTTGTCACAAGTGT
CTTAAATTTTGCTTTTGATAATAATGATTACCACTCATTCAGTACTAACTTACTTGATA
TTAGACACTGCATTAAATACCTTGCAAACATTATTTTGTTTGATCCTGACAACCATAT
GAGATAGGTACTATTCTTATCCATTACCAAAAAAATTAATTTCATGAAGACTTTTCCC
AGAGAGAGAAACTTTAAATATTTACACACACACCTCTCTCCCTGTAACAATTCCGTAG
TCCTGATAACAGCAAATAAGCAAAGTCTGTGTAGGATGCTTTACCAACAGTCCCACC
TAGAGGCAGGAGAGTGAACCAGCTAGAAAATATTTTATTCATATTTCTTCCAGAAAG
GCTCCATTGGAGTTTGAACTCAATTTATGTTATAATTTTCTTATTATTTTTGTATTGGT
TTTCCTGAAACCAATACAAAGTAAGAAAGCATTGGTTCCACTAAAAATGTCCTAAAA
CCAGCCAAGCACAGTGGCTCACACCTATAATCCCAGTACTTTGGGAGGCCGAGGCGG
GTGGATCACTTAAGCCAGGAGTTCAAGACTAGCCTGGCCAACATGACGAAACCCCAT
CTCTACTAAAAATACAAAAATTAGCAGGGTGTGGTAGCACACACCTGTAATCTCAGC
TACTCAGGAAGCTGAGACATGAGAATCGCTTGAACCTCAGAGGCAGAGATTACAGTG
AGCAGAGATCACGCCACTGTACTTCTGCCTGGGTGACAGAGCGAGACTCTATCTAAA
AAAAAATAAACACATAAATAGTAAAATGTCCTGAAACCATTATGGGGTTAAAGCAA
GAGGCAGGGCTGGTTCCCAGGATTTTCTGTCTAATCTCCAGTGAGCCACAGACCTATT
CCTGATCAACTTGAGAATAAACACATCAGTAAAGATGTGTAAGGCTGTCTGACTTTC
CCATTTCTGTAGAATTTTATTTGAAGAGAAGTTTCTCCTTTCTCCAGGCCCCATATTGT
TTATACAAAAAGACCTTTCCAGTAAATGTCCACAACCACTACCATCAACTAAAATGTT
TTCCCACTAATGCTTTCAATGGTAATCAGTATTTAACAGGGCACTTAGGATTATTTTTT
GATCAACCATTGTTTAGATATTCCCACTTATAATTACTCCTGTGAAGGATTGCCTCGG
GGCATCAGCTGATCCTGAGAAATTATCCAGAAGCCATGAGTGTGTAATAATTTAGTC
TTAAACCTAAATAGGTCAGTATTGGGTGGGACTTTTCTCAGCTGCATAATGGGGAGA
ATAAAAAGAATATGGAAAGAAGTTACGTAACACATCCTGGGTCACAAACAGAGGTA
AGACTTGAACACAGGCCTGACATCAAAGCCCATGCCAGTATGACTTACAAAAGGTAG
ACTGGACTACCTGCATTTGAGTCACTAGTGATGCTTATCACTGGGCCTCACCAAAGAA
CCTTGGAATCAGAATCTTTGGAGGTAGATGCCAGGCACCTGCATTGTTATCAAGTGCT
CCAGTGATTACCATTCACTGTACAGAGCCAAACAGACTCCTGATGCTGGAAGAAAAT
TACAGTGCTCAAAGTGCAGGGCAGGGTGTACATCTGGATCTAAATCACTGAGCAACC
ACAGGGTTTCAAGAGAGGGTCAAAACAAGGACTTTCTGCTCTCTGTGGCCAAGGGGA
CACTAAGTTTGCACTGTTCTCAGATCTCCAAAGAGACTTTGGTGTATGGGGGATAGG
GAGGGGGGAAGGGGGTGTGAAATAAAAGGAGAAAGTGAATTTGATTATTTGATTGA
TGAAAATTGAAAAGCTTATTGTAGGGCCTAGCCTACAGTTGATGAAAAAACAATGGA
TCAGGAAGAAGATCAGAACTTGTCTCAGTCCTCAACTGTTTTCCTCAGGCTTTGGTTG
AATATTGCCATCCTGTAATTCATTATAGCATTTTCTGTTGCATAAACGCTTAGCAACA
AAGCCTTTTTTTAAAAAAATTTGTAACTCCTCAATGAGGATTAAATGCTTCTTCTTCTA
AGACAGTCCGAAATATACTCACAGCTGAAAATTCAGCTAACCGCATTTCCCAACTAG
CCACATTCTATAGAAAACTCTAAGCCATGCAGATGAGTACAGACTTGACAATAGTGC
TCAAGGCTGGGAGTACTATTCATCTGAAAAGAATGCTCCCTCCAATTGGTGGGCCGTT
ATTCTGCTAGGTTTGTGTTTGGATAATTATAAGATGGCTATGTTTTTCTTCCCCAGTCT
CAGGAGGCCCAGGTGCTGAAACAATTGGCAGAGAAGAGGGAACACGAGCGAGAAGT
CCTTCAGAAGGCTTTGGAGGAGAACAACAACTTCAGCAAGATGGCGGAGGAAAAGC
TGATCCTGAAAATGGAACAAATTAAGGAAAACCGTGAGGCTAATCTAGCTGCTATTA
TTGAACGTCTGCAGGAAAAGGTAATCTCAGCAGAGTCCTGAGCAGATGGATATATTC
ATATGCAGCACAGCTGGGTGAACTTCCATATGCCTGAGCACAGAGACGAAGTCAAAA
TTTGCTGCAGGTGTGAGGACAACTAACTCCCATGGGCAGGGTCTCACAGTGTAGCAT
TGAGTTAGCAGGAGGTGCAACATGGTAGAGAAATGGGAATCCATCATGAAAGCTGG
AATTTTGTCAAATTTTCCCATGGTGAGTGGATTCAGGGAGGCTGATTCATGCTTTTGA
AATGTGTAAGACTTCTATACAAGCCTCACGAGGCAATCTGTAGGAAAAATGTTACAC
TGGAAATATTAATGTCTATATATTATATTGATATAAGTATAAATAACATTTGATTTAA
TATTTGTTTAATATATGACATTAAATATATATTTAATTAAAATATTAAATTAGAAAAA
TATATTTGCCAGAAAAGGCCAGGGTATTTATGAACACTGGTAAGCCCATTCTAGGGT
ATAATAGCATCACATGGGACCATAGCAAAGATTAGCTCATAGGGGATGTTTCATCCA
GTTCTGGTATCCTGGTGCCCTTCTCTTCAACAACCTAAACATATATTCATTCCCATGA
GTCAGGAGGAGCTGTGCTGGAGTTCTTCTGAAAAATGCTGTCTTTCACTTTTGTACTC
TCTATGCTGTCTCCCACCTATCCCCTCAAAAAACCTTTCCTTTGAAAATATACAGTAT
AGCTGTGAGTAGTTTAGCTGTGTCCGTTTCCAGAAATTGGAATAAGCATTGAGAAAT
GGGATGTTTGAGAAAGACGCCTCAATCCTTTTCTGAGCAGTCAGTCACCCTTCCCGCC
AGTAGCAAGTGCCTTTGTGTGATAGGCATTGGAGATGCAGAGCAAAACAGGAGTGTG
CCTGTCATCAGAGCCCTGAGAGTTTAATTAGATGAGCCTCCTGTTTTCTATTTCTCAG
AGTTTCATGTCTTCTGTTAGAGATGGCCCTTCTCATCTAAGGTTCAAAAAACCTTATC
CTGAAGTTCTGATGATTCTGTTTTCATTCTCAGTCTCTGACTGCAAATATCCAACTAG
AAACAAAGGAAATCAGGCATGAAAACTTTTAAAGATATAATTGCATGGAGATCTTCA
TTTGTGCTCGTGAGGAATTTTTGAAAGCATTGCTGGGGAAGGGTGTGTGGGCTCTGAT
GCAGCAGTAAGACACTGAGGCTCTCAGAGGTCCGTGGACGAGTACTGCTGACTTGGG
CAAGAACCGGAATAGTTACCTGATGCCTTATCCGAAACATGAAAGTTCGGATTAAAT
TTGTATTTATAAGCTAGTGTTTTTATACTCTCAGAACAATGTCATTGCGTTTCACCCAA
GTGAGTCAAGTCACGATTTGGAAGAGGCAACAGAATTTGGCTCTCTCCAGGTGATTT
ATGGCGGTATAGGAACACATGTTTTACTCAGATACAGGGGAGCAAAGTTCCATTTGC
TAAAGTTTACTCCCCTGACCTTCAACCAGTCAGTCTTCCTCCATCTGCCACCACTTTGC
ACTTCTCCAGAGAACTAAGGATGTTCCCGCTTGACCAGTGCTCATAACATGGACAGC
AGAGGGCCACTGTGTGATCTCTTTGAGATCACTGTGACTCAACCTTCTTCTCACATCC
TAGGCCCTAAAACAATTAAGTGAAGTTGCTAGGAACGGTACCTGCTGATCTTATTGC
AGCATTCTCAATTAGGCCTCAATGCAAGATTTATATCACTGGCAGTCCTGGAGCATTT
TTGTTTTTCAAATTACACATACCCAAACACACGGCATAGCCTCCTTTTTTGTTTGTTTG
TTTTTTTGAGATAGAGTCTCGCTGTGTCGCCCAGGCTGGAGTGCAGTGGCACGATCTC
AGCTCACTGCAACCTCTGCCTCCTGGGTTCAAGTGATTCTCATGTCTCAGCCTCCCAA
GTAGCTGAGATTACAGGCGTATACCACCACGCCCAGCTAATTTTTGTATTTTTAGTAG
AGACAGGGTTTTGCCGTGTTGGCCAAGCTGGTCTCAAACTCCTGACCTCAAGTGATCC
ACCCACCTCGGCCTCCCGAAGTGCTGGGATTACAGGTGTGAGCCACCGTGCCCAGCC
AGGGCATATCCTTCTTGATTTCAATTGTAAAATAGTTCAAAAATTTTCCATATTTTATC
TAATATTTCCAGAAGTGCTAGCTTTTAACGGACCATTTTTTTCCTCTGTGTGTTTTTTT
CTCTTCACCTAGCCCAGCCATGCTCAGCTCATTTTTGTACTCTTTCCACTCCCAACCAA
ATTTAGTGCCCTCCCCCATACATGCATACATGTACATCTGCACACCACTTTTCCTGCA
AATAATCAACCCAAAGAGTGCTTAAAATTCCTGACATCAACCCACAGAATCTCCAAG
GATGGGACCCAGCATCCATACATTTTAAAAACTCTCCATATAGTTCCAATATGCAGCC
AGATTTGAGAACTAGTGGTTCGTAGCCTGTTCTGATTTAAATCTCAGCTCTCAGCAGT
CTATCCCACGTCACATAATGCAGCCCAGAGAAATTCTAGGACCACATTTTTTTCTGGT
ATTTCATAGCTAATGAGGTGCTTTTCAAATCTAATAGGATCTTTGGCCAGTGTCAGTC
AAGATCTTTTATCTCCTCAATAAAAAGGAAATACCATATTTACTTTGATTTGATGTAT
ATCACATAGGTGGATTTAATACAAAATTGTGGTTTACATATTGTGAATGTGTATACTA
AAACTACTTTGCTTTTTCCTAAAATAAGACAAAGTTTTATATTGGAAGTAATATTTAG
CATTTTGTTTGAATGAAGTTACTCCTATTAAATTAGAAATTTAAAAGAGGGTCAGTAA
TAACAGTAAAGCCAAAAGGCATGACACTGCCAACGTAACATAAGCTGCTCTGAAATC
TACCATATCAAAAGATAATTATGCTGGGCATGGTGGCTCACACCTGTAATCCCAGCA
CTTTGGGAGGCCAAGGCAAGAGAATTGCTTGAAGCCAGGAGTTCGAGACCAGCCTGG
GAAATATAATGATACCTTGCCTCAAACAAAAATTCAAAAATTAGCCAGCAGTGGTGG
CACACTTGTAAAAATGCCTGTAGTCATAGCTACTTCAGAGGCTGAGATGAAAGGATT
GCTTGGGCCAAGGAGTTCGAGACTGCACTCCAACCTGGGAAATATTGTGCCACTGCA
CTCCAACCTGGGAAACAGAACAAGACCCTGTCTCTAAAATAAAAAGAAAAAAAAAG
ATGACCACTTCTGAAATGACACCTATCAATGAGTTAATCATTCAATGAATATGTATTG
AGTCCCTACTATATGCTTAGGAACCTTTGTAATATCATTACCAACCATGTCTTTCCCA
ATACAGACAATACAAAATTCAGCAATAAATAATATAGCACCAACAATTAGAGAATA
AGACAACATGTAGTATGGTCCAATATAGACAGTAAATACAAAGACACTGAATAATAT
CAGTAAAAGTAAATTCACATCAAGGTCACTACACCATGCGCCCACCCTTATGATAGC
CCTCACTGGCCCTATCAATTAAGCAAGAGACATGATACAACTCTGTGCAAGCTTTTCC
ACAATCTGCCTACCATTCAGCACTCAGTCGCTCTTCCCTTCAATTAAGAGAATTGAGC
ATTCAAGCATATTTTCACCATGATGCCCATAATGGTATCTTCAATGTCACTGACTGAT
AAATTCCCAGAAACCCCTCAGAGCCCCAGCCATGTTAGCTCAAAGCCTTTAGCTAAA
ACTGAAAGCCTAAAGCAAAAGCAGCCCTGGCTGCACTTCGGAATCTACTGGACAGCT
CTTTAAGGGATTCTGATTTAATGTCTGGAATAGGGCCAAGAACCTTGTATTATTTTAA
AGGCTCACTAGTAGGCTCTAATATTTAGCCGTGGTTGAGAACCACTGTGCTAAATGTT
TCTTAAATATGCTTTGTGATGTCATCATAAATTATATTTTAGTATTTTTTGTCTTTGTTG
CATAAGTGTTCTTTCTTCCTCCAAAGAAGAATGTTACACTCATTTCTTATTTCAGTTTC
CTGTTTTCATAGCACCTCATCTTAACACTCCAGGCTATTATATAGAAAAGAATCAAAT
GTGGAGAAGGCTGTGGGAGAAGGGATGCCTGTGCCACAAAGGCCTGCATTAGGCTG
ACCTATTGATGTCATATCCAGGACTCAAAAGACTAGTCTGTGGATTATGACTGGTGA
AGTTCAAAATGTTCTTATTCTTAGAGTGGTATGAGAAGTAGAAAGAGAGAGAAACAG
AGAAGGGGAGGAGAGGGGAAGAGAGGAAGATGAGAGAAAGGAAAGAGAGGGGGA
AACACCTGTTCTTGACATACAGGAATGATTCAAGACATTTTCTTCCTCCCCTGATGTG
TCCCTTTCTCCCCTAACGCACTATGCAGCATCCTGCAGAAAATTCACCACCTGACCCT
TTTAGAAACCCTGAGTAGTAGGAGCGCCAAATGACCCAATCAAGAATTGCAGTGAGA
CAGTTAGTTTTGAAAAATCAGTTAAAGCATGTATAATCATTTTAACAACAATACATCT
ATTCACTAAACATATAATTTTAATGTCAAATATTTACGTGTAAACATATTGACCAATC
TTTCGATGTAGTTGGGCCCAATACCTTTTCCAAAAATTGATCAGTTAATGGGGGTTCT
ATGGGGGTTTCTTTTCTTGCCATTATTCACACTTATGTCACATTAGCTATGATTTGCAG
TTTTAATTTCTTTAAAATTGAGTAGGGACTAAAGACATCTCCAAAAAGCCTGGATATA
GACTTTTTACAACTTTTCCATAGCTTTTATAGTTGACTCACCCAGTATCTACTAAATAC
TTCACTTTCTCACGTATTTCCAAAGGTTTCTCTCCACCCTCACAATTTTCCATTAATGT
AGTACTTAATTAAATTAGATAGTTAAATTTTCAAATGTGAATTGCTAAACAGGTGTGG
AAATACCATTGGCTATAATCAAGCATATAACACAACCATTTGAGAAGGAAAGTATGT
GGCAATATTAGGGAAGAGCCCTTTCCTCTCAAGCAATTCAGCATTTAGGAACCATCA
GACAGCAGGACGATGGAGGGAACAGAGAGGGTTAACATGGCAAGTTACTGAAGAGG
ACTTCTACTGAATCTTGTTGAATTCCCCACTTAATCCAGATTGTATCATATCTTCTTTC
TTTTGTAATTCTACCATATCATCTTAGTCAATGCCAAGACTTCTGAGCTCATAACATG
GTAACAAATACCAAAGGAGCTTTCAGTATCGTTTAGAAAGGAGAGAAGCAAGTAAC
CCAGACAAACTTGACAACTGCTTTCCCCTATCCAACCATGAAGTACAGTACTTAGGA
AATAAAAGAAATTGCTTCACTATAATTCATCATTTCACTTCTAATATCTAGAAAATGT
CAAATGAAAATATTATAGCCATATTTTAGTGGCAATAGTAGCACATAATATGATGCA
ACTTAAAATGATAAAAATATTTTCAGGGAATAAGATTCTGTGATTCTTTCCCTAAGAG
GTAATTTTGATAATATGTACCTGTTTTGTAAATGTCAATAGTCTTGGGGATACAGGTG
GTGTTTGGTTACATGGAAAAGTTCCTTAGTGGTGATTTCTGAGATTTTAGTGCACCCA
ATACCCAAGCAGTGTACACTGTACCCAATATGTAGTCTTTCATCCCTCGCCCCCACTC
CCAACCTTCCCCCACAAGTCTCTAAAGTCCATTATATCACTCTTATATCTTTGCATACT
CATAGCTTAGCTCCCACTTATGAGAACATATGATAGTTAGTGCTCAATTCCTGAGTTA
CTTCACTTAGAATAATGGCCTCCAGCTCCACCCAAGTTGCTGCAAAAGACACTATTTA
GTTCCTTTTTATGGCTGAGTAGTATTACATGGTGTATATATACCACATTTTATTTATCC
ACTTGTTGGTCAATGGACACTTAACATTAGTTCCATATCTTTGTAATTTCAAGTTGTGC
TGCTATAAGCATGCATGAGCCTGTGTCTTTTTCATATAATTACTTCTTTTCCTTTGGGT
AGATACCCAGCAGTGGGATTGCTGGATCAAATGATAGTTCTACTTTCAGTTCTTTATG
TTTTCCACAGTGGTCATACTAATTTACATTCCCATCAACAGTGTAAAGTGTTCCCTTTT
CATCACACCCATGCCAACACCTATTGTTTTTTGACTTTTTAATTACGGCCATTCTTGCA
GGAGTAAGGTGGTATTTCATTGTGGTTTTAATTTGCATTTCCCTGATGTTGACAATATT
TAACTCTTTAGTTATAGATTCCAGCTATTATCAATTTACACCTATTGCATTCTTCTCAT
CTTTTGTTTTCTTGTGATTCTGATGCACAAATATCATTTGTGCAACCACTTACTGTTGA
ACATGTCTGATGAACACTTACTATTGAACATGTCTGATGAATGAATAATGAAATAGG
AAAAGGGATTAAAACTAGCCTTTATTAATTGTTTGCTATAGGCCAGACATTTTTGGAT
GTACTATCACATTTCATCCAAACAACAACCTAAAAGAAAATACTGTGATTATCCCCAT
TTCACATCTAAGGAACCTGGTCTTTAGGAAGATTAAGTCATTTGGCCAAGATCACAA
GTAGACCACAGAGACTAGATTTGAATGCAAGTCTGTTTGACTCCAAACCTTTTTACTA
TCTGCCCATGACCCCTGATCACCAACATCTCAATGTATGAACATGTGCTTTCTTAGCT
CACACAACTCACTCCTGACCCCTTTTTTATATTGCAAGTGCATAGTCATTAGTAAAAA
GAAGGATTTTTGATGATACTGACCTCATCTTGAATTTAATTAGGCTCATATGACAGAA
TTCCATAGATGGAATTGACATCCTAGGTCATATAGTCCAAGTCCTTGTTTATATTTGA
TACCTAGTGAGATTAAAGGGACATTAAAAAGTAAAGAAAGGAAAGACCTCATATTTC
TTACCTTCCAGTAGAGAAATCTTTCTATGAAATCAGAGGAAAGAATTAGAGGACCAG
AATTTTTCCTAAAATCAACTTTCATACATCTTTTTTCATATAAAAGGCATAGCTGCAT
ACAATGCTAAAATATTGTATTACATTTCCTTTATATTGATGGGAGGAAGGGGGTAAAT
TGCAGAAAACATTGTAAATTTAGATATGCTTGGGCCTCTGACAGTGCCTAGCAAATA
TCAGGAGATCAATAATGAAATAAATATTATCAAAGAGTAGTCTTCTTGATGAACCTT
CTCTGAGTATCACAACTGCTTTAGGAACCTCTAGATTCAAGGTCTAGTAATTGCAAAC
AGTGAGCTGATAAGAAAAACAGACTGTATGGGAAATTACATGCTTCCTGCATGACTG
CCTTTTGTTCTCCCACATTTTGATATAAAGTCACATTAACAGTTCATGAGTAAATATTC
GATAATGTGAACGTAAAGTGTTCAAATAATAGAGTGACTAAAATGCCTGAAAACAAA
TAATTTTTAATTAGAAACTCATAATCATTTATTTTCTCTTTTTCCACATTATCTCAAGC
TCACAAATTATATTTATTCTTTCCTATGGCAAAATCCATTTTGTTAACACTAATTTTGA
GTTTAACAAGAAGTGTACTCCAAAGTAGCCTAATAATACTAATTATAATGTTTCCTGC
TATGTTATCAGTTTGAATTTATATGAATCTTTAGACTTGAGGCTTCTTTTTCCTAGCAT
AGTGATGGTCTGGGCTTTTTCTCAATTTTTGCCAGAGCTCAGCTCTCACTAATTAGTTT
CTTTCTGCATGAGAAAAAGATTTTGCTTCATCTTTTTCCTTATAATAGCAGAACAAAA
AGAAGAATCAGCTGCATCCATGCTAATTTCCCCTGTGACATTTCCAAACAGGATTTGA
TTTCTCTATGCATGCCTCTTTCCTTCTCTTCATGGTTTTTGAACATATACAAAAGCTCA
TTTAAACCAATTAAATAAAATTGTTTTTAATCTCTTTCTCTAGAGTCAACTTCCTGCTT
ACTCCAACTCTGTATCTTTGAAGGAAGTATAGGGTGGTCTATGCCTTTTTTCTCCCAG
AATCTACACTTGAAAAGACACATTTTTCCATGCAACTATAAAATGTTCTCCTCACTCA
ACATTGAAATTGTATAGCAGTGATTAAGAGAGTGAGCTGTAGAGCCAGGTTCCCTGG
GTTTAAATCCCACTTGTTAGTATCATGAAGATGGGCAAGTTACTTACCCTTCCTGTGT
TTCAGTTTCTTCATCTGCAAAATGGGGACAATAATAGAATGTCCACTATAAGATTATT
GTGAGGATTAAGGGAATTAATACAGGTAAAACGTGTACTGATGCAGGTCTGGTACAC
ATTAAGTGCCTAATAAATATTCAGTATTATGATATAAAGAACCCTATAAGTGTAGACT
CCTTGAGATTAATAGAGTTTAACGATAAGTTTTACTTTATAGCTGGTCAAGTTTATTT
CTTCTGAACTAAAAGAATCTATAGAGTCTCAATTTCTGGAGCTTCAGAGGGAAGGAG
AGAAGCAATGTAAGCAACATTCTACAGAAATATAAATAATACTACTAATAATTAGCA
TCTTAAAATTTCAATTCAATGAACATTTATTTAGCGCCTATGATATATGCAAGACAGT
TTGATTTTAGTCATCTGATGTATAGCCACATACTAAAAAATACTGATTTTAGTCATCT
GATGTATAGCCACATACTAAAAAATACTTCCTCCATCAGTTCCCTCCTCAGGAAGTTC
AGTTCCCAATCCCAGGCTAGTACCTTGGTTCCTTATGTAAATAAACATCCACCAATTA
CATGCTATCTGCAAAGCACTCTGCTAGGCCCTGCAAATGGAAAAAAAAATGATAAAA
CATAGTCCAGGCCCTCAATGAGCTTACAGTCAAATATAATAGAGGAGACAAGAACAG
AGAGGCTCATAATACAACTAGAATAAAATGACTGCCGAATAAAAGGAAAGATTTAT
GCAGGTGTTCAAATGGAAAGTGAGATAAGTTTGCAGGTTAGTCTTTGCAGTCTCATA
AAAATCTTTATGGAGAAAAGGACAATGGTCATAGGGCTTAAAGAGTAAGTTTATAAT
CCTGACCAGTGGAGATGAAAGACTAGCATTGAAAATTGCATGACAAGACAATTCCAT
TAAACTGAAACATCAAGTGTGTGTAGGAAAAGATGGGGGTTATGACTGGAAACGTCA
CTTGGACTGCAATTATGAAGGGCCTTGACAAACAGGTCAAGAGTTTAAGAAGCAGTA
TAGAAAGTCTTCGTCCTGGATCTAGCCCTCCCAGAGTGTCCATCAGGATTATAAAGTC
CTTAAAATATTAGTCAAAAGGAACGACATCATTAGAAATGATAGAGAAACAATAATG
TGATGTTTTATTACCTTTCTCTGGATTTATACTCTGATCCTAATATTCAAAACTATCTT
AATAACATGAACTTTTGGTCATAGTTTTAAACAAAAACAGTGTTAAATATATTTTTTA
AAACACAGTAAGTCTTGTAAGATCTTTTCTAACATGACATTTTGCAGGGCCCATATTT
TCCTTCTGAAATGGGAAAAATTCATAAAAGTAGACACCAAACTGGGTTACTTCTAGT
CAAGCGCATGGTACGCAAAGGACCAGACAAAAAGGGCCTGTGACATTTCTTCTTCCT
TTTGTGTTTTTTAGGAGAGGCATGCTGCGGAGGTGCGCAGGAACAAGGAACTCCAGG
TTGAACTGTCTGGCTGAAGCAAGGGAGGGTCTGGCACGCCCCACCAATAGTAAATCC
CCCTGCCTATATTATAATGGATCATGCGATATCAGGATGGGGAATGTATGACATGGTT
TAAAAAGAACTCATTATAAAAAAAAAAAAACAAAAAAAATCAAAAATTAAAAAAAA
TCAATGCGGTCTCTTTGCAGAATGTTTTGCTTGATGTTTAAAAAATACCTTGGATCTT
ATTTTGTAAATACTTACATTTTTGTTAAAAAATACAAGTATTGCATTATGCAAGTTAT
TTCATAATCTTACATGTCCTGTAACAGGCTTTTGATGTTGTGTCTTTCCACTCAAATGA
ATTTGCTAGGTCTGTTCTTTTTGAAGCTCCCCATGTCTAACTCCATTCCAAAAGAAAA
ATGAGGTCAGTAGACAGTCTATGGTGCTAGAAACCCACCATTGCCTAATGACCTAGA
AGGCTTTGTTGTCTCTGAGCTTGACTAAGACCATACCTAGATCACAGGTATTATGACT
CCACATGAACCTTCACATTTGTTCGCTCATAATCTACTTACTGCCTAAAAACTACAAA
ACCAGGCTAAGAAATACCACCAGTCATAGCATTTACTTCTGCTTCTCCTGGATTATGT
GCTACAAATGTGCTTTGGCTTTAGAAAGGGATGGATGAGAAGACAGACCTGAGACCA
ATCTGGGTAGAAGCAAAAAGTTGAACCTTTTAAAGTGCTGAACACAAATCCAAATTC
GAATGGTTCAAGCAGCCGTGAAATCGCTCTTCATAAAGTGGGCTTAATTCTCTAGTTT
AAGTTCTTTTGATGGAATGAATTAATTAATGTGTCAGGTGGCTTATTTGTGGATGCCA
TGATTGATGATGTTCATTTTAAGCTCTTACCTATAGTACAAGTACATGATGCTACTGA
ATATTTTTCCACTTGGAAACTGTGAGCTGGTTGTTGCATTAAAACACACATACAAACA
AAATCAAAAACACTGCGGACTTTCACTCAAGCTGGTCTTTCTTCCCCAGTGTAAGGCA
ATCCTGCCTACTAACAACACCAACAACAAAACACTCCATCTGTGAAGCTGACGCAGT
TAAGGGGGCTAGGCAGGGCATTTGTGCCAACTAAGAATCACCAGATACCCACCATAA
GTACCTATCGCAGTTTTGAAGTCGTTTCTCCCCAACTCCCAACTCCTGAAGGTTGCTG
CCTGCATATTTACTCTTCATTAGTGCTATTTTCCTGTATGTCATTGTGAGCAAGCTGTG

A STMN2 cryptic exon sequence within the STMN2 transcript is provided as SEQ ID NO: 447.

(SEQ ID NO: 447)
GACTCGGCAGAAGACCTTCGAGAGAAAGGTAGAAAATAAGAATTTGGCTC
TCTGTGTGAGCATGTGTGCGTGTGTGCGAGAGAGAGAGACAGACAGCCTG
CCTAAGAAGAAATGAATGTGAATGCGGCTTGTGGCACAGTTGACAAGGAT
GATAAATCAATAATGCAAGCTTACTATCATTTATGAATAGCAATACTGAA
GAAATTAAAACAAAAGATTGCTGTCTC
(Source: NCBI Reference Sequence: NC_000008.11)

In various embodiments, the STMN2 transcript with a cryptic exon shares between 90-100% identity with SEQ ID NO: 944. In various embodiments, the STMN2 transcript with a cryptic exon shares at least 90%, 91%, 92%, 93%, 94%, 95%, 96%, 97%, 98%, 99% or 100% identity with SEQ ID NO: 944.

### STMN2 Antisense Oligonucleotides Targeting Portions of the STMN2 Transcript

In various embodiments, STMN2 AON disclosed herein target specific portions of STMN2 transcripts that include a cryptic exon. SEQ ID NO: 944, shown above, describes one example of a STMN2 transcript that includes a cryptic exon. In some embodiments, a STMN2 transcript that includes a cryptic exon may share at least 80%, 85%, 90%, 95%, or 100% identity with the nucleobase sequence of SEQ ID NO: 944.

In some embodiments, an STMN2 AON targets a specific portion of the STMN2 transcript, the specific portion of the STMN2 transcript having a length of 10 nucleobases. In some embodiments, an STMN2 AON targets a specific portion of the STMN2 transcript, the specific portion of the STMN2 transcript having a length of 11, 12, 13, 14, 15, 16, 17, 18, 19, 20, 21, 22, 23, 24, or 25 nucleobases in length.

In some embodiments, an STMN2 AON targets a specific portion of the STMN2 transcript, the specific portion of the STMN2 transcript comprising any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. In some embodiments, an STMN2 AON targets a specific portion of the STMN2 transcript, the specific portion of the STMN2 transcript comprising any one of positions 144-164, 144-166, 145-167, 146-166, 146-168, 147-165, or 148-168 of SEQ ID NO: 944. In some embodiments, an STMN2 AON targets a specific portion of the STMN2 transcript, the specific portion of the STMN2 transcript comprising any one of positions 173-191, 173-193, 173-195, 173-197, 175-195, 175-197, 177-197, or 179-197 of SEQ ID NO: 944. In some embodiments, an STMN2 AON targets a specific portion of the STMN2 transcript, the specific portion of the STMN2 transcript comprising any one of positions 185-205, 187-209, 189-209, 185-207, 197-217, 197-219, or 191-209 of SEQ ID NO: 944. In some embodiments, an STMN2 AON targets a specific portion of the STMN2 transcript, the specific portion of the STMN2 transcript comprising any one of positions 237-255, 237-257, 237-259, 239-259, 239-261, 241-261, 237-257, 249-269, 249-271, 252-272, 252-274, or 243-261 of SEQ ID NO: 944.

In some embodiments, an STMN2 AON targets a specific portion of the STMN2 transcript, the specific portion of the STMN2 transcript consisting of any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. In some embodiments, an STMN2 AON targets a specific portion of the STMN2 transcript, the specific portion of the STMN2 transcript consisting of any one of positions 144-164, 144-166, 145-167, 146-166, 146-168, 147-165, or 148-168 of SEQ ID NO: 944. In some embodiments, an STMN2 AON targets a specific portion of the STMN2 transcript, the specific portion of the STMN2 transcript consisting of any one of positions 173-191, 173-193, 173-195, 173-197, 175-195, 175-197, 177-197, or 179-197 of SEQ ID NO: 944. In some embodiments, an STMN2 AON targets a specific portion of the STMN2 transcript, the specific portion of the STMN2 transcript consisting of any one of positions 185-205, 187-209, 189-209, 185-207, 197-217, 197-219, or 191-209 of SEQ ID NO: 944. In some embodiments, an STMN2 AON targets a specific portion of the STMN2 transcript, the specific portion of the STMN2 transcript consisting of any one of positions 237-255, 237-257, 237-259, 239-259, 239-261, 241-261, 237-257, 249-269, 249-271, 252-272, 252-274, or 243-261 of SEQ ID NO: 944.

In various embodiments, the STMN2 AON comprises a nucleobase sequence that comprises a portion of at least 10 contiguous nucleobases that is complementary to an equal length portion of nucleobases within any one of positions 144-164, 144-166, 145-167, 146-166, 146-168, 147-165, 148-168, 173-191, 173-193, 173-195, 173-197, 175-195, 175-197, 177-197, 179-197, 185-205, 185-207, 197-217, 197-219, 187-209, 189-209, 191-209, 237-255, 237-257, 237-259, 239-259, 239-261, 241-261, 237-257, 249-269, 249-271, 252-272, 252-274, or 243-261 of SEQ ID NO: 944. In various embodiments, the STMN2 AON comprises a nucleobase sequence that comprises a portion of at least 11, 12, 13, 14, 15, 16, 17, 18, 19, 20, 21, 22, 23, 24, or 25 contiguous nucleobases that is complementary to an equal length portion of nucleobases within any one of positions 144-164, 144-166, 145-167, 146-166, 146-168, 147-165, 148-168, 173-191, 173-193, 173-195, 173-197, 175-195, 175-197, 177-197, 179-197, 185-205, 185-207, 197-217, 197-219, 187-209, 189-209, 191-209, 237-255, 237-257, 237-259, 239-259, 239-261, 241-261, 237-257, 249-269, 249-271, 252-272, 252-274, or 243-261 of SEQ ID NO: 944.

In various embodiments, the oligonucleotide comprises linked nucleosides with at least a 19 contiguous nucleobase sequence that is at least 90% (e.g., 90%, 91%, 92%, 93%, 94%, 95%, 96%, 97%, 98%, 99% or 100%) complementary to an equal length portion of a transcript with at least 90% identity (e.g., 90%, 91%, 92%, 93%, 94%, 95%, 96%, 97%, 98%, 99%, or 100% identity) to SEQ ID NO: 944, or to a contiguous 19 to 50 nucleobase portion of SEQ ID NO: 944, wherein at least one nucleoside linkage of the linked nucleosides is a non-natural linkage. In various embodiments, the oligonucleotide comprises linked nucleosides with at least a 19, 20, 21, 22, 23, 24, or 25 contiguous nucleobase sequence that is at least 90% complementary (e.g., 90%, 91%, 92%, 93%, 94%, 95%, 96%, 97%, 98%, 99%, or 100% complementary) to an equal length portion of a transcript with at least 90% (e.g., 90%, 91%, 92%, 93%, 94%, 95%, 96%, 97%, 98%, 99% or 100%) identity to SEQ ID NO: 944, or to a contiguous 19 to 50 nucleobase portion of SEQ ID NO: 944, wherein at least one nucleoside linkage of the linked nucleosides is a non-natural linkage.

In various embodiments, the oligonucleotide comprises linked nucleosides with at least a 19 contiguous nucleobase sequence that comprises a portion of at least 10 contiguous nucleobases that shares at least 90% identity (e.g., 90%, 91%, 92%, 93%, 94%, 95%, 96%, 97%, 98%, 99%, or 100% identity) with an equal length portion of any one of SEQ ID NOs: 1-446, SEQ ID NOs: 894-918, SEQ ID NOs: 945-1390, or SEQ ID NOs: 1392-1432. In various embodiments, the oligonucleotide comprises linked nucleosides with at least a 19 contiguous nucleobase sequence that comprises a portion of at least 11, 12, 13, 14, 15, 16, 17, 18, 19, 20, 21, 22, 23, 24, or 25 contiguous nucleobases that shares at least 90% identity (e.g., 90%, 91%, 92%, 93%, 94%, 95%, 96%, 97%, 98%, 99%, or 100% identity) with an equal length portion of any one of SEQ ID NOs: 1-446, SEQ ID NOs: 894-918, SEQ ID NOs: 945-1390, or SEQ ID NOs: 1392-1432.

In various embodiments, the oligonucleotide comprises linked nucleosides with at least a 19 contiguous nucleobase sequence that comprises a portion of at least 10 contiguous nucleobases that shares at least 90% identity (e.g., 90%, 91%, 92%, 93%, 94%, 95%, 96%, 97%, 98%, 99%, or 100% identity) with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344, wherein at least one nucleoside linkage of the linked nucleosides is a non-natural linkage. In various embodiments, the oligonucleotide comprises linked nucleosides with at least a 19 contiguous nucleobase sequence that comprises a portion of at least 11, 12, 13, 14, 15, 16, 17, 18, 19, 20, 21, 22, 23, 24, or 25 contiguous nucleobases that shares at least 90% identity (e.g., 90%, 91%, 92%, 93%, 94%, 95%, 96%, 97%, 98%, 99%, or 100% identity) with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344, wherein at least one nucleoside linkage of the linked nucleosides is a non-natural linkage.

In various embodiments, the oligonucleotide comprises linked nucleosides with at least a 19 contiguous nucleobase sequence, wherein the nucleobase sequence comprises a portion of at least 10 contiguous nucleobases that shares at least 90% identity (e.g., 90%, 91%, 92%, 93%, 94%, 95%, 96%, 97%, 98%, 99%, or 100% identity) to an equal length portion of any one of SEQ ID NOs: 894-918 or SEQ ID NOs: 1392-1432. In various embodiments, the oligonucleotide comprises linked nucleosides with at least a 20, 21, 22, 23, 24, or 25 contiguous nucleobase sequence, wherein the nucleobase sequence comprises a portion of at least 10 contiguous nucleobases that shares at least 90% identity (e.g., 90%, 91%, 92%, 93%, 94%, 95%, 96%, 97%, 98%, 99%, or 100% identity) to an equal length portion of any one of SEQ ID NOs: 894-918 or SEQ ID NOs: 1392-1432.

In various embodiments, the oligonucleotide comprises linked nucleosides with at least a 19 contiguous nucleobase sequence, wherein the nucleobase sequence comprises a portion of at least 10 contiguous nucleobases that shares at least 90% identity (e.g., 90%, 91%, 92%, 93%, 94%, 95%, 96%, 97%, 98%, 99%, or 100% identity) to an equal length portion of any one of SEQ ID NOs: 894-918 or SEQ ID NOs: 1392-1432. In various embodiments, the oligonucleotide comprises linked nucleosides with at least a 20, 21, 22, 23, 24, or 25 contiguous nucleobase sequence, wherein the nucleobase sequence comprises a portion of at least 10 contiguous nucleobases that shares at least 90% identity (e.g., 90%, 91%, 92%, 93%, 94%, 95%, 96%, 97%, 98%, 99%, or 100% identity) to an equal length portion of any one of SEQ ID NOs: 894-918 or SEQ ID NOs: 1392-1432.

In various embodiments, the oligonucleotide comprises linked nucleosides with at least a 19 contiguous nucleobase sequence, the nucleobase sequence comprising a portion of at least 11, 12, 13, 14, 15, 16, 17, 18, 19, 20, 21, 22, 23, 24, or 25 contiguous nucleobases that shares at least 90% identity (e.g., 90%, 91%, 92%, 93%, 94%, 95%, 96%, 97%, 98%, 99%, or 100% identity) to of any one of SEQ ID NOs: 894-918 or SEQ ID NOs: 1392-1432.

In various embodiments, the nucleobase sequence comprises a portion of at least 10 contiguous nucleobases that is at least 90% complementary (e.g., 90%, 91%, 92%, 93%, 94%, 95%, 96%, 97%, 98%, 99%, or 100% complementary) to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. In various embodiments, the nucleobase sequence comprises a portion of at least 11, 12, 13, 14, 15, 16, 17, 18, 19, 20, 21, 22, 23, 24, or 25 contiguous nucleobases that is at least 90% complementary (e.g., 90%, 91%, 92%, 93%, 94%, 95%, 96%, 97%, 98%, 99%, or 100% complementary) to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944.

In various embodiments, the nucleobase sequence comprises a portion of at least 10 contiguous nucleobases that is at least 90% complementary (e.g., 90%, 91%, 92%, 93%, 94%, 95%, 96%, 97%, 98%, 99%, or 100% complementary) to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. In various embodiments, the nucleobase sequence comprises a portion of at least 11, 12, 13, 14, 15, 16, 17, 18, 19, 20, 21, 22, 23, 24, or 25 contiguous nucleobases that is at least 90% complementary (e.g., 90%, 91%, 92%, 93%, 94%, 95%, 96%, 97%, 98%, 99%, or 100% complementary) to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944.

In various embodiments, the portion of the nucleobase sequence is 100% complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944. In various embodiments, the portion of the nucleobase sequence is 100% complementary to an equal length portion of nucleobases within any one of positions 144-164, 144-166, 145-167, 146-166, 146-168, 147-165, or 148-168 of SEQ ID NO: 944. In various embodiments, the portion of the nucleobase sequence is 100% complementary to an equal length portion of nucleobases within any one of positions 173-191, 173-193, 173-195, 173-197, 175-195, 175-197, 177-197, or 179-197 of SEQ ID NO: 944. In various embodiments, the portion of the nucleobase sequence is 100% complementary to an equal length portion of nucleobases within any one of positions 185-205, 187-209, 189-209, 185-207, 197-217, 197-219, or 191-209 of SEQ ID NO: 944. In various embodiments, the portion of the nucleobase sequence is 100% complementary to an equal length portion of nucleobases within any one of positions 237-255, 237-257, 237-259, 239-259, 239-261, 241-261, 237-257, 249-269, 249-271, 252-272, 252-274, or 243-261 of SEQ ID NO: 944.

### STMN2 Antisense Oligonucleotide Variants

In various embodiments, STMN2 AONs include different variants, hereafter referred to as STMN2 AON variants. A STMN2 AON variant may be an oligonucleotide sequence of 5 to 100 nucleotides in length, for example, 10 to 40 nucleotides in length, for example, 14 to 40 nucleotides in length, 10 to 30 nucleotides in length, for example, 14 to 30 nucleotides in length, for example, 16 to 28 nucleotides in length, for example, 19 to 23 nucleotides in length, for example, 21 to 23 nucleotides in length, for example, or 18, 19, 20, 21, 22, 23, 24, 25, 26, 27, 28, 29, or 30 nucleotides in length. A STMN2 AON variant may be an oligonucleotide sequence complementary to a portion of a STMN2 pre-mRNA sequence or a STMN2 gene sequence.

In various embodiments, a STMN2 AON variant represents a modified version of a corresponding STMN2 AON that includes a nucleobase sequence selected from any one of SEQ ID NOs: 1-446 or SEQ ID NOs: 945-1390. In some embodiments, a STMN2 AON variant includes a nucleobase sequence that represents a shortened version of a nucleobase sequence of a STMN2 AON selected from any one of SEQ ID NOs: 1-446 OR SEQ ID NOs: 945-1390. As one example, if a STMN2 AON includes a 25 mer (e.g., 25 nucleotides in length) a variant (e.g., a STMN2 variant) may include a shorter version (e.g., 15 mer, 16 mer, 17 mer, 18 mer, 19 mer, 20 mer, 21 mer, 22 mer, 23 mer, or 24 mer) of the 25 mer STMN2 AON. In one embodiment, a nucleobase sequence of a STMN2 AON variant differs from a corresponding nucleobase sequence of a STMN2 AON in that 1, 2, 3, 4, 5, or 6 nucleotides are removed from one or both of the 3′ and 5′ ends of the nucleobase sequence of the STMN2 AON. In one embodiment, the corresponding STMN2 AON variant may include a 23 mer where two nucleotides were removed from one of the 3′ or 5′ end of a 25 mer included in the STMN2 AON. In one embodiment, the corresponding STMN2 AON variant may include a 23 mer where one nucleotide is removed from each of the 3′ and 5′ ends of the 25 mer included in the STMN2 AON. In one embodiment, the corresponding STMN2 AON variant may include a 21 mer where two nucleotides are removed from each of the 3′ and 5′ ends of the 25 mer included in the STMN2 AON. In one embodiment, the corresponding STMN2 AON variant may include a 21 mer where four nucleotides are removed from either the 3′ or 5′ end of the 25 mer included in the STMN2 AON. In one embodiment, the corresponding STMN2 AON variant may include a 19 mer where three nucleotides are removed from each of the 3′ and 5′ ends of the 25 mer included in the STMN2 AON. In one embodiment, the corresponding STMN2 AON variant may include a 19 mer where six nucleotides are removed from either the 3′ or 5′ end of the 25 mer included in the STMN2 AON.

Example sequences of STMN2 AON variants are shown below in Table 3. The example STMN2 AON variants are each associated with an identifier that describes the differences between the STMN2 AON variant and the corresponding STMN2 AON. As an example, a STMN2 AON variant includes SEQ ID NO: 894 and is identified using identifier: QSN-144-1/5-1/3. This first portion of the identifier “QSN-144” indicates that the STMN2 AON variant is a modified version of the QSN-144 STMN2 AON which includes SEQ ID NO: 144. Additionally, the second portion of the identifier which includes the numerical indicators of “1/5-1/3” indicate that one nucleotide is removed from each of the 5′ end and the 3′ end of the nucleobase sequence included in the QSN-144 STMN2 AON (e.g., 1 nucleotide removed from each of 3′ and 5′ end of SEQ ID NO: 144). To provide another example, a STMN2 AON variant includes SEQ ID NO: 895 and is identified as QSN-144-2/3. This STMN2 AON variant is a modified version of the QSN-144 STMN2 AON. The numerical indicators of “2/3” indicate that two nucleotides are removed from the 3′ end of the nucleobase sequence of the QSN-144 STMN2 AON (e.g., 2 bases removed from 3′ end of SEQ ID NO: 144).

In some embodiments, a STMN2 AON variant differs from a corresponding STMN2 AON in that one or more internucleoside linkages of the STMN2 AON variant are phosphodiester bonds. In such embodiments, the length of the STMN2 AON variant may be the same length as the corresponding STMN2 AON (e.g., 25 nucleotides in length). In some embodiments, the phosphodiester internucleoside linkages connect two, three, four, five, six, seven, eight, nine, or ten contiguous nucleotides.

In some embodiments, the phosphodiester internucleoside linkages connect nucleotides located at one or both of the 3′ or 5′ ends. For example, two, three, four, five, six, seven, eight, nine, or ten contiguous nucleotides at one or both of the 3′ or 5′ ends are connected via phosphodiester internucleoside linkages.

In some embodiments, the phosphodiester internucleoside linkages connect nucleotides located within the nucleobase sequence. For example, within a 25 mer STMN2 AON variant, contiguous nucleotides between positions 6-15 may be connected through phosphodiester internucleoside linkages. In some embodiments, contiguous nucleotides between any one of positions 7-15, 8-14, or 9-13 are connected through phosphodiester internucleoside linkages.

Table 5 below identifies variants of STMN2 AON sequences:

TABLE 5
STMN2 Antisense Oligonucleotide Variant Sequences

| SEQ | | | SEQ | |
|---|---|---|---|---|
| ID | | AON Sequence* | ID | Target Sequence |
| NO: | Identifier | (5′ → 3′) | NO: | (5′ → 3′) |
| 894 | QSN- | ATCCAATTAAGAGAGAGTGATGG | 919 | CCATCACTCTCTCTTAATTGGAT |
| | 144-1/5- | | | |
| | 1/3 | | | |
| 895 | QSN- | AATCCAATTAAGAGAGAGTGATG | 920 | CATCACTCTCTCTTAATTGGATT |
| | 144-2/3 | | | |
| 896 | QSN- | TCCAATTAAGAGAGAGTGATGGG | 921 | CCCATCACTCTCTCTTAATTGGA |
| | 144-2/5 | | | |
| 897 | QSN- | TCCAATTAAGAGAGAGTGATG | 922 | CATCACTCTCTCTTAATTGGA |
| | 144-2/5- | | | |
| | 2/3 | | | |
| 898 | QSN- | CCAATTAAGAGAGAGTGAT | 923 | ATCACTCTCTCTTAATTGG |
| | 144-3/5- | | | |
| | 3/3 | | | |
| 899 | QSN- | AATCCAATTAAGAGAGAGTGA | 924 | TCACTCTCTCTTAATTGGATT |
| | 144-4/3 | | | |
| 900 | QSN- | CAATTAAGAGAGAGTGATGGG | 925 | CCCATCACTCTCTCTTAATTG |
| | 144-4/5 | | | |
| 901 | QSN- | GAGTCCTGCAATATGAATATAAT | 926 | ATTATATTCATATTGCAGGACTC |
| | 173-2/3 | | | |
| 902 | QSN- | GTCCTGCAATATGAATATAATTT | 927 | AAATTATATTCATATTGCAGGAC |
| | 173-2/5 | | | |
| 903 | QSN- | GTCCTGCAATATGAATATAAT | 928 | ATTATATTCATATTGCAGGAC |
| | 173-2/5- | | | |
| | 2/3 | | | |
| 904 | QSN- | GAGTCCTGCAATATGAATATA | 929 | TATATTCATATTGCAGGACTC |
| | 173-4/3 | | | |
| 905 | QSN- | CCTGCAATATGAATATAATTT | 930 | AAATTATATTCATATTGCAGG |
| | 173-4/5 | | | |
| 906 | QSN- | GAGTCCTGCAATATGAATA | 931 | TATTCATATTGCAGGACTC |
| | 173-6/3 | | | |
| 907 | QSN- | TGCAATATGAATATAATTT | 932 | AAATTATATTCATATTGCA |
| | 173-6/5 | | | |
| 908 | QSN- | GTCTTCTGCCGAGTCCTGCAATA | 933 | TATTGCAGGACTCGGCAGAAGAC |
| | 185-2/5 | | | |
| 909 | QSN- | AGGTCTTCTGCCGAGTCCTGC | 934 | GCAGGACTCGGCAGAAGACCT |
| | 185-4/3 | | | |
| 910 | QSN- | CTTCTGCCGAGTCCTGCAATA | 935 | TATTGCAGGACTCGGCAGAAG |
| | 185-4/5 | | | |
| 911 | QSN- | TCTGCCGAGTCCTGCAATA | 936 | TATTGCAGGACTCGGCAGA |
| | 185-6/5 | | | |
| 912 | QSN- | GCACACATGCTCACACAGAGAGC | 937 | GCTCTCTGTGTGAGCATGTGTGC |
| | 237-2/3 | | | |
| 913 | QSN- | ACACATGCTCACACAGAGAGCCA | 938 | TGGCTCTCTGTGTGAGCATGTGT |
| | 237-2/5 | | | |
| 914 | QSN- | ACACATGCTCACACAGAGAGC | 939 | GCTCTCTGTGTGAGCATGTGT |
| | 237-2/5- | | | |
| | 2/3 | | | |
| 915 | QSN- | GCACACATGCTCACACAGAGA | 940 | TCTCTGTGTGAGCATGTGTGC |
| | 237-4/3 | | | |
| 916 | QSN- | ACATGCTCACACAGAGAGCCA | 941 | TGGCTCTCTGTGTGAGCATGT |
| | 237-4/5 | | | |
| 917 | QSN- | GCACACATGCTCACACAGA | 942 | TCTGTGTGAGCATGTGTGC |
| | 237-6/3 | | | |
| 918 | QSN- | ATGCTCACACAGAGAGCCA | 943 | TGGCTCTCTGTGTGAGCAT |
| | 237-6/5 | | | |
| 1417 | QSN- | G-A-G-TCCTGCAATATGAATATAA-T-T-T¹ | 620 | AAATTATATTCATATTGCAGGACTC |
| | 173-po3 | | | |
| 1418 | QSN- | GAGTCCTG-C-A-A-T-A-TGAATATAATTT¹ | 620 | AAATTATATTCATATTGCAGGACTC |
| | 173-po5 | | | |
| 1419 | QSN- | A-A-T-CCAATTAAGAGAGAGTGAT-G-G-G¹ | 591 | CCCATCACTCTCTCTTAATTGGATT |
| | 144-po3 | | | |
| 1420 | QSN- | AATCCAAT-T-A-A-G-A-GAGAGTGATGGG¹ | 591 | CCCATCACTCTCTCTTAATTGGATT |
| | 144-po5 | | | |
| 1421 | QSN- | A-G-G-TCTTCTGCCGAGTCCTGCA-A-T-A¹ | 633 | ATTGCAGGACTCGGCAGAAGACCTT |
| | 185-po3 | | | |
| 1422 | QSN- | AGGTCTTC-T-G-C-C-G-AGTCCTGCAATA¹ | 633 | ATTGCAGGACTCGGCAGAAGACCTT |
| | 185-po5 | | | |
| 1423 | QSN- | G-C-A-CACATGCTCACACAGAGAG-C-C-A¹ | 684 | TGGCTCTCTGTGTGAGCATGTGTGC |
| | 237-po3 | | | |
| 1424 | QSN- | GCACACAT-G-C-T-C-A-CACAGAGAGCCA¹ | 684 | TGGCTCTCTGTGTGAGCATGTGTGC |
| | 237-po5 | | | |

*Except where noted to the contrary (e.g., in SEQ ID NOs: 1417, 1418, 1419, 1420, 1421, 1422, 1423, and 1424 in Table 5), at least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments, except where noted to the contrary (e.g., in SEQ ID NOs: 1417, 1418, 1419, 1420, 1421, 1422, 1423, and 1424 in Table 5), every nucleoside linkage is a phosphorothioate linkage.
¹The notation “-” indicates the presence of a phosphodiester linkage in SEQ ID NOs: 1417, 1418, 1419, 1420, 1421, 1422, 1423, and 1424 in Table 5.

Table 6 below identifies additional variants of STMN2 AON sequences:

TABLE 6
Additional STMN2 Antisense Oligonucleotide Variant
Sequences

| SEQ | | |
|---|---|---|
| ID | | AON Sequence* |
| NO: | Identifier | (5′ → 3′) |
| 1392 | QSN-144-1/5- | AUCCAAUUAAGAGAGAGUGAUGG |
| | 1/3 | |
| 1393 | QSN-144-2/3 | AAUCCAAUUAAGAGAGAGUGAUG |
| 1394 | QSN-144-2/5 | UCCAAUUAAGAGAGAGUGAUGGG |
| 1395 | QSN-144-2/5- | UCCAAUUAAGAGAGAGUGAUG |
| | 2/3 | |
| 1396 | QSN-144-3/5- | CCAAUUAAGAGAGAGUGAU |
| | 3/3 | |
| 1397 | QSN-144-4/3 | AAUCCAAUUAAGAGAGAGUGA |
| 1398 | QSN-144-4/5 | CAAUUAAGAGAGAGUGAUGGG |
| 1399 | QSN-173-2/3 | GAGUCCUGCAAUAUGAAUAUAAU |
| 1400 | QSN-173-2/5 | GUCCUGCAAUAUGAAUAUAAUUU |
| 1401 | QSN-173-2/5- | GUCCUGCAAUAUGAAUAUAAU |
| | 2/3 | |
| 1402 | QSN-173-4/3 | GAGUCCUGCAAUAUGAAUAUA |
| 1403 | QSN-173-4/5 | CCUGCAAUAUGAAUAUAAUUU |
| 1404 | QSN-173-6/3 | GAGUCCUGCAAUAUGAAUA |
| 1405 | QSN-173-6/5 | UGCAAUAUGAAUAUAAUUU |
| 1406 | QSN-185-2/5 | GUCUUCUGCCGAGUCCUGCAAUA |
| 1407 | QSN-185-4/3 | AGGUCUUCUGCCGAGUCCUGC |
| 1408 | QSN-185-4/5 | CUUCUGCCGAGUCCUGCAAUA |
| 1409 | QSN-185-6/5 | UCUGCCGAGUCCUGCAAUA |
| 1410 | QSN-237-2/3 | GCACACAUGCUCACACAGAGAGC |
| 1411 | QSN-237-2/5 | ACACAUGCUCACACAGAGAGCCA |
| 1412 | QSN-237-2/5- | ACACAUGCUCACACAGAGAGC |
| | 2/3 | |
| 1413 | QSN-237-4/3 | GCACACAUGCUCACACAGAGA |
| 1414 | QSN-237-4/5 | ACAUGCUCACACAGAGAGCCA |
| 1415 | QSN-237-6/3 | GCACACAUGCUCACACAGA |
| 1416 | QSN-237-6/5 | AUGCUCACACAGAGAGCCA |
| 1425 | QSN-173-po3 | G-A-G-UCCUGCAAUAUGAAUAUAA-U-U-U¹ |
| 1426 | QSN-173-po5 | GAGUCCUG-C-A-A-U-A-UGAAUAUAAUUU¹ |
| 1427 | QSN-144-po3 | A-A-U-CCAAUUAAGAGAGAGUGAU-G-G-G¹ |
| 1428 | QSN-144-po5 | AAUCCAAU-U-A-A-G-A-GAGAGUGAUGGG¹ |
| 1429 | QSN-185-po3 | A-G-G-UCUUCUGCCGAGUCCUGCA-A-U-A¹ |
| 1430 | QSN-185-po5 | AGGUCUUC-U-G-C-C-G-AGUCCUGCAAUA¹ |
| 1431 | QSN-237-po3 | G-C-A-CACAUGCUCACACAGAGAG-C-C-A¹ |
| 1432 | QSN-237-po5 | GCACACAU-G-C-U-C-A-CACAGAGAGCCA¹ |

*Except where noted to the contrary (e.g., in SEQ ID NOs: 1425, 1426, 1427, 1428, 1429, 1430, 1431, and 1432 in Table 6), at least one nucleoside linkage of the nucleobase sequence is selected from a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments, except where noted to the contrary (e.g., in SEQ ID NOs: 1425, 1426, 1427, 1428, 1429, 1430, 1431, and 1432 in Table 6), every nucleoside linkage is a phosphorothioate linkage.
¹The notation “-”indicates the presence of a phosphodiester linkage in SEQ ID NOs: 1425, 1426, 1427, 1428, 1429, 1430, 1431, and 1432 in Table 6.

### Performance of STMN2 Antisense Oligonucleotides and Variants

Generally, STMN2 AON and STMN2 AON variants can target STMN2 transcripts with a cryptic exon in order to increase, restore, rescue, or stabilize levels of expression of STMN2 mRNA that is capable of translation to produce a functional STMN2 protein (e.g., full length STMN2). In various embodiments, STMN2 AON and STMN2 AON variants can exhibit at least a 60%, 70%, 80%, or 90% increase of full length STMN2 protein. In various embodiments, STMN2 AON and STMN2 AON variants can exhibit at least a 100%, 200%, 300%, or 400% increase of full length STMN2 protein. In some embodiments, the percent increase of the full length STMN2 protein is an increase in comparison to a reduced level of full length STMN2 protein achieved using a TDP43 antisense oligonucleotide. For example, a TDP43 antisense oligonucleotide can be used to deplete full length STMN2 protein followed by increase of the full length STMN2 protein using a STMN2 AON or STMN2 AON variant.

In some embodiments, STMN2 AON and STMN2 AON variants reduce levels of STMN2 transcript with a cryptic exon. In various embodiments, STMN2 AON and STMN2 AON variants can exhibit at least a 50%, 60%, 70%, 80%, 90%, 95%, 96%, 97%, 98%, or 99% reduction of the STMN2 transcript with the cryptic exon. In some embodiments, the percent reduction of cryptic exon levels is a decrease in comparison to an increased level of cryptic exon achieved using a TDP43 antisense oligonucleotide. For example, a TDP43 antisense oligonucleotide can be used to increase cryptic exon levels followed by a reduction of cryptic exon levels using a STMN2 AON or STMN2 AON variant.

In some embodiments, STMN2 AON and STMN2 AON variants can exhibit at least a 10%, 20%, 30%, 40%, 50%, 60%, 70%, 80%, 90%, or 100% rescue of full length STMN2 protein. In some embodiments, the percent rescue of full length STMN2 refers to the % of full length STMN2 following depletion using a TDP43 antisense oligonucleotide and a treatment using STMN2 AON or STMN2 AON variant in comparison to a negative control (e.g., cells that did not undergo depletion or treatment or cells that were treated with a vehicle solution).

In some embodiments, STMN2 AON and AON variants exhibit between 50% to 100% rescue of full length STMN2. In some embodiments, STMN2 AON and AON variants exhibit between 60% to 100% rescue of full length STMN2. In some embodiments, STMN2 AON and AON variants exhibit between 70% to 100% rescue of full length STMN2. In some embodiments, STMN2 AON and AON variants exhibit between 80% to 100% rescue of full length STMN2. In some embodiments, STMN2 AON and AON variants exhibit between 90% to 100% rescue of full length STMN2. In some embodiments, STMN2 AON and AON variants exhibit between 60% to 90% rescue of full length STMN2. In some embodiments, STMN2 AON and AON variants exhibit between 50% to 80% rescue of full length STMN2. In some embodiments, STMN2 AON and AON variants exhibit between 60% to 80% rescue of full length STMN2.

In particular embodiments, QSN-31 STMN2 AON (SEQ ID NO: 31) exhibits between 50 to 80% rescue of full length STMN2. In particular embodiments, QSN-31 STMN2 AON (SEQ ID NO: 31) exhibits between 60 to 90% rescue of full length STMN2. In particular embodiments, QSN-31 STMN2 AON (SEQ ID NO: 31) exhibits between 70 to 100% rescue of full length STMN2. In some embodiments, all internucleoside linkages of the QSN-31 STMN2 AON (SEQ ID NO: 31) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-36 STMN2 AON (SEQ ID NO: 36) exhibits between 50 to 80% rescue of full length STMN2. In particular embodiments, QSN-36 STMN2 AON (SEQ ID NO: 36) exhibits between 60 to 90% rescue of full length STMN2. In particular embodiments, QSN-36 STMN2 AON (SEQ ID NO: 36) exhibits between 70 to 100% rescue of full length STMN2. In some embodiments, all internucleoside linkages of the QSN-36 STMN2 AON (SEQ ID NO: 36) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-36 STMN2 AON (SEQ ID NO: 36) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-41 STMN2 AON (SEQ ID NO: 41) exhibits between 50 to 80% rescue of full length STMN2. In particular embodiments, QSN-41 STMN2 AON (SEQ ID NO: 41) exhibits between 60 to 90% rescue of full length STMN2. In particular embodiments, QSN-41 STMN2 AON (SEQ ID NO: 41) exhibits between 70 to 100% rescue of full length STMN2. In some embodiments, all internucleoside linkages of the QSN-41 STMN2 AON (SEQ ID NO: 41) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-46 STMN2 AON (SEQ ID NO: 46) exhibits between 50 to 80% rescue of full length STMN2. In particular embodiments, QSN-46 STMN2 AON (SEQ ID NO: 46) exhibits between 60 to 90% rescue of full length STMN2. In particular embodiments, QSN-46 STMN2 AON (SEQ ID NO: 46) exhibits between 70 to 100% rescue of full length STMN2. In some embodiments, all internucleoside linkages of the QSN-46 STMN2 AON (SEQ ID NO: 46) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-55 STMN2 AON (SEQ ID NO: 55) exhibits between 50 to 80% rescue of full length STMN2. In particular embodiments, QSN-55 STMN2 AON (SEQ ID NO: 55) exhibits between 60 to 90% rescue of full length STMN2. In particular embodiments, QSN-55 STMN2 AON (SEQ ID NO: 55) exhibits between 70 to 100% rescue of full length STMN2. In some embodiments, all internucleoside linkages of the QSN-55 STMN2 AON (SEQ ID NO: 55) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-55 STMN2 AON (SEQ ID NO: 55) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-144 STMN2 AON (SEQ ID NO: 144) exhibits between 50 to 80% rescue of full length STMN2. In particular embodiments, QSN-144 STMN2 AON (SEQ ID NO: 144) exhibits between 60 to 90% rescue of full length STMN2. In particular embodiments, QSN-144 STMN2 AON (SEQ ID NO: 144) exhibits between 70 to 100% rescue of full length STMN2. In some embodiments, all internucleoside linkages of the QSN-144 STMN2 AON (SEQ ID NO: 144) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-144 STMN2 AON (SEQ ID NO: 144) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-146 STMN2 AON (SEQ ID NO: 146) exhibits between 50 to 80% rescue of full length STMN2. In particular embodiments, QSN-146 STMN2 AON (SEQ ID NO: 146) exhibits between 60 to 90% rescue of full length STMN2. In particular embodiments, QSN-146 STMN2 AON (SEQ ID NO: 146) exhibits between 70 to 100% rescue of full length STMN2. In some embodiments, all internucleoside linkages of the QSN-146 STMN2 AON (SEQ ID NO: 146) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-150 STMN2 AON (SEQ ID NO: 150) exhibits between 50 to 80% rescue of full length STMN2. In particular embodiments, QSN-150 STMN2 AON (SEQ ID NO: 150) exhibits between 60 to 90% rescue of full length STMN2. In particular embodiments, QSN-150 STMN2 AON (SEQ ID NO: 150) exhibits between 70 to 100% rescue of full length STMN2. In some embodiments, all internucleoside linkages of the QSN-150 STMN2 AON (SEQ ID NO: 150) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-169 STMN2 AON (SEQ ID NO: 169) exhibits between 50 to 80% rescue of full length STMN2. In particular embodiments, QSN-169 STMN2 AON (SEQ ID NO: 169) exhibits between 60 to 90% rescue of full length STMN2. In particular embodiments, QSN-169 STMN2 AON (SEQ ID NO: 169) exhibits between 70 to 100% rescue of full length STMN2. In some embodiments, all internucleoside linkages of the QSN-169 STMN2 AON (SEQ ID NO: 169) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-170 STMN2 AON (SEQ ID NO: 170) exhibits between 50 to 80% rescue of full length STMN2. In particular embodiments, QSN-170 STMN2 AON (SEQ ID NO: 170) exhibits between 60 to 90% rescue of full length STMN2. In particular embodiments, QSN-170 STMN2 AON (SEQ ID NO: 170) exhibits between 70 to 100% rescue of full length STMN2. In some embodiments, all internucleoside linkages of the QSN-170 STMN2 AON (SEQ ID NO: 170) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-171 STMN2 AON (SEQ ID NO: 171) exhibits between 50 to 80% rescue of full length STMN2. In particular embodiments, QSN-171 STMN2 AON (SEQ ID NO: 171) exhibits between 60 to 90% rescue of full length STMN2. In particular embodiments, QSN-171 STMN2 AON (SEQ ID NO: 171) exhibits between 70 to 100% rescue of full length STMN2. In some embodiments, all internucleoside linkages of the QSN-171 STMN2 AON (SEQ ID NO: 171) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-172 STMN2 AON (SEQ ID NO: 172) exhibits between 50 to 80% rescue of full length STMN2. In particular embodiments, QSN-172 STMN2 AON (SEQ ID NO: 172) exhibits between 60 to 90% rescue of full length STMN2. In particular embodiments, QSN-172 STMN2 AON (SEQ ID NO: 172) exhibits between 70 to 100% rescue of full length STMN2. In some embodiments, all internucleoside linkages of the QSN-172 STMN2 AON (SEQ ID NO: 172) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-173 STMN2 AON (SEQ ID NO: 173) exhibits between 50 to 80% rescue of full length STMN2. In particular embodiments, QSN-173 STMN2 AON (SEQ ID NO: 173) exhibits between 60 to 90% rescue of full length STMN2. In particular embodiments, QSN-173 STMN2 AON (SEQ ID NO: 173) exhibits between 70 to 100% rescue of full length STMN2. In some embodiments, all internucleoside linkages of the QSN-173 STMN2 AON (SEQ ID NO: 173) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-173 STMN2 AON (SEQ ID NO: 173) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-177 STMN2 AON (SEQ ID NO: 177) exhibits between 50 to 80% rescue of full length STMN2. In particular embodiments, QSN-177 STMN2 AON (SEQ ID NO: 177) exhibits between 60 to 90% rescue of full length STMN2. In particular embodiments, QSN-177 STMN2 AON (SEQ ID NO: 177) exhibits between 70 to 100% rescue of full length STMN2. In some embodiments, all internucleoside linkages of the QSN-177 STMN2 AON (SEQ ID NO: 177) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-177 STMN2 AON (SEQ ID NO: 177) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In particular embodiments, QSN-181 STMN2 AON (SEQ ID NO: 181) exhibits between 50 to 80% rescue of full length STMN2. In particular embodiments, QSN-181 STMN2 AON (SEQ ID NO: 181) exhibits between 60 to 90% rescue of full length STMN2. In particular embodiments, QSN-181 STMN2 AON (SEQ ID NO: 181) exhibits between 70 to 100% rescue of full length STMN2. In some embodiments, all internucleoside linkages of the QSN-181 STMN2 AON (SEQ ID NO: 181) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-181 STMN2 AON (SEQ ID NO: 181) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-185 STMN2 AON (SEQ ID NO: 185) exhibits between 50 to 80% rescue of full length STMN2. In particular embodiments, QSN-185 STMN2 AON (SEQ ID NO: 185) exhibits between 60 to 90% rescue of full length STMN2. In particular embodiments, QSN-185 STMN2 AON (SEQ ID NO: 185) exhibits between 70 to 100% rescue of full length STMN2. In some embodiments, all internucleoside linkages of the QSN-185 STMN2 AON (SEQ ID NO: 185) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In some embodiments, all internucleoside linkages of the QSN-185 STMN2 AON (SEQ ID NO: 185) oligonucleotide are phosphorothioate linkages, and each of the linked nucleosides of the oligonucleotide are 2′-O-(2-methoxyethyl) (2′-MOE) nucleosides. In particular embodiments, QSN-197 STMN2 AON (SEQ ID NO: 197) exhibits between 50 to 80% rescue of full length STMN2. In particular embodiments, QSN-197 STMN2 AON (SEQ ID NO: 197) exhibits between 60 to 90% rescue of full length STMN2. In particular embodiments, QSN-197 STMN2 AON (SEQ ID NO: 197) exhibits between 70 to 100% rescue of full length STMN2. In some embodiments, all internucleoside linkages of the QSN-197 STMN2 AON (SEQ ID NO: 197) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-197 STMN2 AON (SEQ ID NO: 197) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-203 STMN2 AON (SEQ ID NO: 203) exhibits between 50 to 80% rescue of full length STMN2. In particular embodiments, QSN-203 STMN2 AON (SEQ ID NO: 203) exhibits between 60 to 90% rescue of full length STMN2. In particular embodiments, QSN-203 STMN2 AON (SEQ ID NO: 203) exhibits between 70 to 100% rescue of full length STMN2. In some embodiments, all internucleoside linkages of the QSN-203 STMN2 AON (SEQ ID NO: 203) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-203 STMN2 AON (SEQ ID NO: 203) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In particular embodiments, QSN-209 STMN2 AON (SEQ ID NO: 209) exhibits between 50 to 80% rescue of full length STMN2. In particular embodiments, QSN-209 STMN2 AON (SEQ ID NO: 209) exhibits between 60 to 90% rescue of full length STMN2. In some embodiments, all internucleoside linkages of the QSN-209 STMN2 AON (SEQ ID NO: 209) oligonucleotide are phosphorothioate linkages, and each of the linked nucleosides of the oligonucleotide are 2′-O-(2-methoxyethyl) (2′-MOE) nucleosides. In particular embodiments, QSN-209 STMN2 AON (SEQ ID NO: 209) exhibits between 70 to 100% rescue of full length STMN2. In some embodiments, all internucleoside linkages of the QSN-209 STMN2 AON (SEQ ID NO: 209) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-215 STMN2 AON (SEQ ID NO: 215) exhibits between 50 to 80% rescue of full length STMN2. In particular embodiments, QSN-215 STMN2 AON (SEQ ID NO: 215) exhibits between 60 to 90% rescue of full length STMN2. In particular embodiments, QSN-215 STMN2 AON (SEQ ID NO: 215) exhibits between 70 to 100% rescue of full length STMN2. In some embodiments, all internucleoside linkages of the QSN-215 STMN2 AON (SEQ ID NO: 215) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-215 STMN2 AON (SEQ ID NO: 215) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In particular embodiments, QSN-237 STMN2 AON (SEQ ID NO: 237) exhibits between 50 to 80% rescue of full length STMN2. In particular embodiments, QSN-237 STMN2 AON (SEQ ID NO: 237) exhibits between 60 to 90% rescue of full length STMN2. In particular embodiments, QSN-237 STMN2 AON (SEQ ID NO: 237) exhibits between 70 to 100% rescue of full length STMN2. In some embodiments, all internucleoside linkages of the QSN-237 STMN2 AON (SEQ ID NO: 237) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-237 STMN2 AON (SEQ ID NO: 237) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-244 STMN2 AON (SEQ ID NO: 244) exhibits between 50 to 80% rescue of full length STMN2. In particular embodiments, QSN-244 STMN2 AON (SEQ ID NO: 244) exhibits between 60 to 90% rescue of full length STMN2. In particular embodiments, QSN-244 STMN2 AON (SEQ ID NO: 244) exhibits between 70 to 100% rescue of full length STMN2. In some embodiments, all internucleoside linkages of the QSN-244 STMN2 AON (SEQ ID NO: 244) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-244 STMN2 AON (SEQ ID NO: 244) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-249 STMN2 AON (SEQ ID NO: 249) exhibits between 50 to 80% rescue of full length STMN2. In particular embodiments, QSN-249 STMN2 AON (SEQ ID NO: 249) exhibits between 60 to 90% rescue of full length STMN2. In particular embodiments, QSN-249 STMN2 AON (SEQ ID NO: 249) exhibits between 70 to 100% rescue of full length STMN2. In some embodiments, all internucleoside linkages of the QSN-249 STMN2 AON (SEQ ID NO: 249) oligonucleotide are phosphorothioate linkages, each of the linked nucleosides of the oligonucleotide are 2′-O-(2-methoxyethyl) (2′-MOE) nucleosides, and each “C” is replaced with a 5-MeC.

In particular embodiments, QSN-252 STMN2 AON (SEQ ID NO: 252) exhibits between 50 to 80% rescue of full length STMN2. In particular embodiments, QSN-252 STMN2 AON (SEQ ID NO: 252) exhibits between 60 to 90% rescue of full length STMN2. In particular embodiments, QSN-252 STMN2 AON (SEQ ID NO: 252) exhibits between 70 to 100% rescue of full length STMN2. In some embodiments, all internucleoside linkages of the QSN-252 STMN2 AON (SEQ ID NO: 252) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-252 STMN2 AON (SEQ ID NO: 252) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-380 STMN2 AON (SEQ ID NO: 380) exhibits between 50 to 80% rescue of full length STMN2. In particular embodiments, QSN-380 STMN2 AON (SEQ ID NO: 380) exhibits between 60 to 90% rescue of full length STMN2. In particular embodiments, QSN-380 STMN2 AON (SEQ ID NO: 380) exhibits between 70 to 100% rescue of full length STMN2. In some embodiments, all internucleoside linkages of the QSN-380 STMN2 AON (SEQ ID NO: 380) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-380 STMN2 AON (SEQ ID NO: 380) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In particular embodiments, QSN-385 STMN2 AON (SEQ ID NO: 385) exhibits between 50 to 80% rescue of full length STMN2. In particular embodiments, QSN-385 STMN2 AON (SEQ ID NO: 385) exhibits between 60 to 90% rescue of full length STMN2. In particular embodiments, QSN-385 STMN2 AON (SEQ ID NO: 385) exhibits between 70 to 100% rescue of full length STMN2. In some embodiments, all internucleoside linkages of the QSN-385 STMN2 AON (SEQ ID NO: 385) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-385 STMN2 AON (SEQ ID NO: 385) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-390 STMN2 AON (SEQ ID NO: 390) exhibits between 50 to 80% rescue of full length STMN2. In particular embodiments, QSN-390 STMN2 AON (SEQ ID NO: 390) exhibits between 60 to 90% rescue of full length STMN2. In particular embodiments, QSN-390 STMN2 AON (SEQ ID NO: 390) exhibits between 70 to 100% rescue of full length STMN2. In some embodiments, all internucleoside linkages of the QSN-390 STMN2 AON (SEQ ID NO: 390) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-390 STMN2 AON (SEQ ID NO: 390) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In particular embodiments, QSN-395 STMN2 AON (SEQ ID NO: 395) exhibits between 50 to 80% rescue of full length STMN2. In particular embodiments, QSN-395 STMN2 AON (SEQ ID NO: 395) exhibits between 60 to 90% rescue of full length STMN2. In particular embodiments, QSN-395 STMN2 AON (SEQ ID NO: 395) exhibits between 70 to 100% rescue of full length STMN2. In some embodiments, all internucleoside linkages of the QSN-395 STMN2 AON (SEQ ID NO: 395) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-395 STMN2 AON (SEQ ID NO: 395) oligonucleotide are phosphorothioate linkages, and each of the linked nucleosides of the oligonucleotide are 2′-O-(2-methoxyethyl) (2′-MOE) nucleosides and each “C” is replaced with a 5-MeC. In particular embodiments, QSN-400 STMN2 AON (SEQ ID NO: 400) exhibits between 50 to 80% rescue of full length STMN2. In particular embodiments, QSN-400 STMN2 AON (SEQ ID NO: 400) exhibits between 60 to 90% rescue of full length STMN2. In particular embodiments, QSN-400 STMN2 AON (SEQ ID NO: 400) exhibits between 70 to 100% rescue of full length STMN2. In some embodiments, all internucleoside linkages of the QSN-400 STMN2 AON (SEQ ID NO: 400) oligonucleotide are phosphorothioate linkages, and each of the linked nucleosides of the oligonucleotide are 2′-O-(2-methoxyethyl) (2′-MOE) nucleosides. In some embodiments, all internucleoside linkages of the QSN-400 STMN2 AON (SEQ ID NO: 400) oligonucleotide are phosphorothioate linkages, and each of the linked nucleosides of the oligonucleotide are 2′-O-(2-methoxyethyl) (2′-MOE) nucleosides and each “C” is replaced with a 5-MeC.

In particular embodiments, QSN-144-1/5-1/3 (SEQ ID NO: 894) exhibits between 30 to 100% rescue of full length STMN2. In particular embodiments, QSN-144-1/5-1/3 (SEQ ID NO: 894) exhibits between 40 to 80% rescue of full length STMN2. In particular embodiments, QSN-144-1/5-1/3 (SEQ ID NO: 894) exhibits between 50 to 60% rescue of full length STMN2.

In particular embodiments, QSN-144-2/3 (SEQ ID NO: 895) exhibits between 30 to 100% rescue of full length STMN2. In particular embodiments, QSN-144-2/3 (SEQ ID NO: 895) exhibits between 40 to 80% rescue of full length STMN2. In particular embodiments, QSN-144-2/3 (SEQ ID NO: 895) exhibits between 50 to 60% rescue of full length STMN2. In some embodiments, all internucleoside linkages of the QSN-144-2/3 STMN2 AON (SEQ ID NO: 895) oligonucleotide are phosphorothioate linkages, and each of the linked nucleosides of the oligonucleotide are 2′-O-(2-methoxyethyl) (2′-MOE) nucleosides.

In particular embodiments, QSN-144-2/5 (SEQ ID NO: 896) exhibits between 30 to 100% rescue of full length STMN2. In particular embodiments, QSN-144-2/5 (SEQ ID NO: 896) exhibits between 40 to 80% rescue of full length STMN2. In particular embodiments, QSN-144-2/5 (SEQ ID NO: 896) exhibits between 50 to 60% rescue of full length STMN2. In some embodiments, all internucleoside linkages of the QSN-144-2/5 (SEQ ID NO: 896) oligonucleotide are phosphorothioate linkages, and each of the linked nucleosides of the oligonucleotide are 2′-O-(2-methoxyethyl) (2′-MOE) nucleosides.

In particular embodiments, QSN-144-2/5-2/3 (SEQ ID NO: 897) exhibits between 30 to 100% rescue of full length STMN2. In particular embodiments, QSN-144-2/5-2/3 (SEQ ID NO: 897) exhibits between 40 to 80% rescue of full length STMN2. In particular embodiments, QSN-144-2/5-2/3 (SEQ ID NO: 897) exhibits between 50 to 60% rescue of full length STMN2. In some embodiments, all internucleoside linkages of the QSN-144-2/5-2/3 STMN2 AON (SEQ ID NO: 897) oligonucleotide are phosphorothioate linkages, and each of the linked nucleosides of the oligonucleotide are 2′-O-(2-methoxyethyl) (2′-MOE) nucleosides.

In particular embodiments, QSN-144-3/5-3/3 (SEQ ID NO: 898) exhibits between 30 to 100% rescue of full length STMN2. In particular embodiments, QSN-144-3/5-3/3 (SEQ ID NO: 898) exhibits between 40 to 80% rescue of full length STMN2. In particular embodiments, QSN-144-3/5-3/3 (SEQ ID NO: 898) exhibits between 50 to 60% rescue of full length STMN2. In some embodiments, all internucleoside linkages of the QSN-144-3/5-3/3 (SEQ ID NO: 898) oligonucleotide are phosphorothioate linkages, and each of the linked nucleosides of the oligonucleotide are 2′-O-(2-methoxyethyl) (2′-MOE) nucleosides.

In particular embodiments, QSN-144-4/3 (SEQ ID NO: 899) exhibits between 30 to 100% rescue of full length STMN2. In particular embodiments, QSN-144-4/3 (SEQ ID NO: 899) exhibits between 40 to 80% rescue of full length STMN2. In particular embodiments, QSN-144-4/3 (SEQ ID NO: 899) exhibits between 50 to 60% rescue of full length STMN2. In some embodiments, all internucleoside linkages of the QSN-144-4/3 STMN2 AON (SEQ ID NO: 899) oligonucleotide are phosphorothioate linkages, and each of the linked nucleosides of the oligonucleotide are 2′-O-(2-methoxyethyl) (2′-MOE) nucleosides.

In particular embodiments, QSN-144-4/5 (SEQ ID NO: 900) exhibits between 30 to 100% rescue of full length STMN2. In particular embodiments, QSN-144-4/5 (SEQ ID NO: 900) exhibits between 40 to 80% rescue of full length STMN2. In particular embodiments, QSN-144-4/5 (SEQ ID NO: 900) exhibits between 50 to 60% rescue of full length STMN2. In some embodiments, all internucleoside linkages of the QSN-144-4/5 (SEQ ID NO: 900) oligonucleotide are phosphorothioate linkages, and each of the linked nucleosides of the oligonucleotide are 2′-O-(2-methoxyethyl) (2′-MOE) nucleosides.

In particular embodiments, QSN-173-2/3 (SEQ ID NO: 901) exhibits between 30 to 100% rescue of full length STMN2. In particular embodiments, QSN-173-2/3 (SEQ ID NO: 901) exhibits between 40 to 80% rescue of full length STMN2. In particular embodiments, QSN-173-2/3 (SEQ ID NO: 901) exhibits between 50 to 60% rescue of full length STMN2. In some embodiments, all internucleoside linkages of the QSN-173-2/3 STMN2 AON (SEQ ID NO: 901) oligonucleotide are phosphorothioate linkages, and each of the linked nucleosides of the oligonucleotide are 2′-O-(2-methoxyethyl) (2′-MOE) nucleosides.

In particular embodiments, QSN-173-2/5 (SEQ ID NO: 902) exhibits between 30 to 100% rescue of full length STMN2. In particular embodiments, QSN-173-2/5 (SEQ ID NO: 902) exhibits between 40 to 80% rescue of full length STMN2. In particular embodiments, QSN-173-2/5 (SEQ ID NO: 902) exhibits between 50 to 60% rescue of full length STMN2. In some embodiments, all internucleoside linkages of the QSN-173-2/5 (SEQ ID NO: 902) oligonucleotide are phosphorothioate linkages, and each of the linked nucleosides of the oligonucleotide are 2′-O-(2-methoxyethyl) (2′-MOE) nucleosides.

In particular embodiments, QSN-173-2/5-2/3 (SEQ ID NO: 903) exhibits between 30 to 100% rescue of full length STMN2. In particular embodiments, QSN-173-2/5-2/3 (SEQ ID NO: 903) exhibits between 40 to 80% rescue of full length STMN2. In particular embodiments, QSN-173-2/5-2/3 (SEQ ID NO: 903) exhibits between 50 to 60% rescue of full length STMN2. In some embodiments, all internucleoside linkages of the QSN-173-2/5-2/3 (SEQ ID NO: 903) oligonucleotide are phosphorothioate linkages, and each of the linked nucleosides of the oligonucleotide are 2′-O-(2-methoxyethyl) (2′-MOE) nucleosides.

In particular embodiments, QSN-173-4/3 (SEQ ID NO: 904) exhibits between 30 to 100% rescue of full length STMN2. In particular embodiments, QSN-173-4/3 (SEQ ID NO: 904) exhibits between 40 to 80% rescue of full length STMN2. In particular embodiments, QSN-173-4/3 (SEQ ID NO: 904) exhibits between 50 to 60% rescue of full length STMN2. In some embodiments, all internucleoside linkages of the QSN-173-4/3 (SEQ ID NO: 904) oligonucleotide are phosphorothioate linkages, and each of the linked nucleosides of the oligonucleotide are 2′-O-(2-methoxyethyl) (2′-MOE) nucleosides.

In particular embodiments, QSN-173-4/5 (SEQ ID NO: 905) exhibits between 30 to 100% rescue of full length STMN2. In particular embodiments, QSN-173-4/5 (SEQ ID NO: 905) exhibits between 40 to 80% rescue of full length STMN2. In particular embodiments, QSN-173-4/5 (SEQ ID NO: 905) exhibits between 50 to 60% rescue of full length STMN2. In some embodiments, all internucleoside linkages of the QSN-173-4/5 (SEQ ID NO: 905) oligonucleotide are phosphorothioate linkages, and each of the linked nucleosides of the oligonucleotide are 2′-O-(2-methoxyethyl) (2′-MOE) nucleosides.

In particular embodiments, QSN-173-6/3 (SEQ ID NO: 906) exhibits between 30 to 100% rescue of full length STMN2. In particular embodiments, QSN-173-6/3 (SEQ ID NO: 906) exhibits between 40 to 80% rescue of full length STMN2. In particular embodiments, QSN-173-6/3 (SEQ ID NO: 906) exhibits between 50 to 60% rescue of full length STMN2. In some embodiments, all internucleoside linkages of the QSN-173-6/3 (SEQ ID NO: 906) oligonucleotide are phosphorothioate linkages, and each of the linked nucleosides of the oligonucleotide are 2′-O-(2-methoxyethyl) (2′-MOE) nucleosides.

In particular embodiments, QSN-173-6/5 (SEQ ID NO: 907) exhibits between 30 to 100% rescue of full length STMN2. In particular embodiments, QSN-173-6/5 (SEQ ID NO: 907) exhibits between 40 to 80% rescue of full length STMN2. In particular embodiments, QSN-173-6/5 (SEQ ID NO: 907) exhibits between 50 to 60% rescue of full length STMN2. In some embodiments, all internucleoside linkages of the QSN-173-6/5 (SEQ ID NO: 907) oligonucleotide are phosphorothioate linkages, and each of the linked nucleosides of the oligonucleotide are 2′-O-(2-methoxyethyl) (2′-MOE) nucleosides.

In particular embodiments, QSN-185-2/5 (SEQ ID NO: 908) exhibits between 30 to 100% rescue of full length STMN2. In particular embodiments, QSN-185-2/5 (SEQ ID NO: 908) exhibits between 40 to 80% rescue of full length STMN2. In particular embodiments, QSN-185-2/5 (SEQ ID NO: 908) exhibits between 50 to 60% rescue of full length STMN2. In some embodiments, all internucleoside linkages of the QSN-185-2/5 (SEQ ID NO: 908) oligonucleotide are phosphorothioate linkages, and each of the linked nucleosides of the oligonucleotide are 2′-O-(2-methoxyethyl) (2′-MOE) nucleosides.

In particular embodiments, QSN-185-4/3 (SEQ ID NO: 909) exhibits between 30 to 100% rescue of full length STMN2. In particular embodiments, QSN-185-4/3 (SEQ ID NO: 909) exhibits between 40 to 80% rescue of full length STMN2. In particular embodiments, QSN-185-4/3 (SEQ ID NO: 909) exhibits between 50 to 60% rescue of full length STMN2. In some embodiments, all internucleoside linkages of the QSN-185-4/3 (SEQ ID NO: 909) oligonucleotide are phosphorothioate linkages, and each of the linked nucleosides of the oligonucleotide are 2′-O-(2-methoxyethyl) (2′-MOE) nucleosides.

In particular embodiments, QSN-185-4/5 (SEQ ID NO: 910) exhibits between 30 to 100% rescue of full length STMN2. In particular embodiments, QSN-185-4/5 (SEQ ID NO: 910) exhibits between 40 to 80% rescue of full length STMN2. In particular embodiments, QSN-185-4/5 (SEQ ID NO: 910) exhibits between 50 to 60% rescue of full length STMN2. In some embodiments, all internucleoside linkages of the QSN-185-4/5 (SEQ ID NO: 910) oligonucleotide are phosphorothioate linkages, and each of the linked nucleosides of the oligonucleotide are 2′-O-(2-methoxyethyl) (2′-MOE) nucleosides.

In particular embodiments, QSN-185-6/5 (SEQ ID NO: 911) exhibits between 30 to 100% rescue of full length STMN2. In particular embodiments, QSN-185-6/5 (SEQ ID NO: 911) exhibits between 40 to 80% rescue of full length STMN2. In particular embodiments, QSN-185-6/5 (SEQ ID NO: 911) exhibits between 50 to 60% rescue of full length STMN2. In some embodiments, all internucleoside linkages of the QSN-185-6/5 (SEQ ID NO: 911) oligonucleotide are phosphorothioate linkages, and each of the linked nucleosides of the oligonucleotide are 2′-O-(2-methoxyethyl) (2′-MOE) nucleosides.

In particular embodiments, QSN-237-2/3 (SEQ ID NO: 912) exhibits between 30 to 100% rescue of full length STMN2. In particular embodiments, QSN-237-2/3 (SEQ ID NO: 912) exhibits between 40 to 80% rescue of full length STMN2. In particular embodiments, QSN-237-2/3 (SEQ ID NO: 912) exhibits between 50 to 60% rescue of full length STMN2. In some embodiments, all internucleoside linkages of the QSN-237-2/3 (SEQ ID NO: 912) oligonucleotide are phosphorothioate linkages, and each of the linked nucleosides of the oligonucleotide are 2′-O-(2-methoxyethyl) (2′-MOE) nucleosides.

In particular embodiments, QSN-237-2/5 (SEQ ID NO: 913) exhibits between 30 to 100% rescue of full length STMN2. In particular embodiments, QSN-237-2/5 (SEQ ID NO: 913) exhibits between 40 to 80% rescue of full length STMN2. In particular embodiments, QSN-237-2/5 (SEQ ID NO: 913) exhibits between 50 to 60% rescue of full length STMN2. In some embodiments, all internucleoside linkages of the QSN-237-2/5 (SEQ ID NO: 913) oligonucleotide are phosphorothioate linkages, and each of the linked nucleosides of the oligonucleotide are 2′-O-(2-methoxyethyl) (2′-MOE) nucleosides.

In particular embodiments, QSN-237-2/5-2/3 (SEQ ID NO: 914) exhibits between 30 to 100% rescue of full length STMN2. In particular embodiments, QSN-237-2/5-2/3 (SEQ ID NO: 914) exhibits between 40 to 80% rescue of full length STMN2. In particular embodiments, QSN-237-2/5-2/3 (SEQ ID NO: 914) exhibits between 50 to 60% rescue of full length STMN2. In some embodiments, all internucleoside linkages of the QSN-237-2/5-2/3 (SEQ ID NO: 914) oligonucleotide are phosphorothioate linkages, and each of the linked nucleosides of the oligonucleotide are 2′-O-(2-methoxyethyl) (2′-MOE) nucleosides.

In particular embodiments, QSN-237-4/3 (SEQ ID NO: 915) exhibits between 30 to 100% rescue of full length STMN2. In particular embodiments, QSN-237-4/3 (SEQ ID NO: 915) exhibits between 40 to 80% rescue of full length STMN2. In particular embodiments, QSN-237-4/3 (SEQ ID NO: 915) exhibits between 50 to 60% rescue of full length STMN2. In some embodiments, all internucleoside linkages of the QSN-237-4/3 (SEQ ID NO: 915) oligonucleotide are phosphorothioate linkages, and each of the linked nucleosides of the oligonucleotide are 2′-O-(2-methoxyethyl) (2′-MOE) nucleosides.

In particular embodiments, QSN-237-4/5 (SEQ ID NO: 916) exhibits between 30 to 100% rescue of full length STMN2. In particular embodiments, QSN-237-4/5 (SEQ ID NO: 916) exhibits between 40 to 80% rescue of full length STMN2. In particular embodiments, QSN-237-4/5 (SEQ ID NO: 916) exhibits between 50 to 60% rescue of full length STMN2. In some embodiments, all internucleoside linkages of the QSN-237-4/5 (SEQ ID NO: 916) oligonucleotide are phosphorothioate linkages, and each of the linked nucleosides of the oligonucleotide are 2′-O-(2-methoxyethyl) (2′-MOE) nucleosides.

In particular embodiments, QSN-237-6/3 (SEQ ID NO: 917) exhibits between 30 to 100% rescue of full length STMN2. In particular embodiments, QSN-237-6/3 (SEQ ID NO: 917) exhibits between 40 to 80% rescue of full length STMN2. In particular embodiments, QSN-237-6/3 (SEQ ID NO: 917) exhibits between 50 to 60% rescue of full length STMN2. In some embodiments, all internucleoside linkages of the QSN-237-6/3 (SEQ ID NO: 917) oligonucleotide are phosphorothioate linkages, and each of the linked nucleosides of the oligonucleotide are 2′-O-(2-methoxyethyl) (2′-MOE) nucleosides.

In particular embodiments, QSN-237-6/5 (SEQ ID NO: 918) exhibits between 30 to 100% rescue of full length STMN2. In particular embodiments, QSN-237-6/5 (SEQ ID NO: 918) exhibits between 40 to 80% rescue of full length STMN2. In particular embodiments, QSN-237-6/5 (SEQ ID NO: 918) exhibits between 50 to 60% rescue of full length STMN2. In some embodiments, all internucleoside linkages of QSN-237-6/5 (SEQ ID NO: 918) oligonucleotide are phosphorothioate linkages, and each of the linked nucleosides of the oligonucleotide are 2′-O-(2-methoxyethyl) (2′-MOE) nucleosides.

In particular embodiments, QSN-173-po3 (SEQ ID NO: 1417) exhibits between 30 to 100% rescue of full length STMN2. In particular embodiments, QSN-173-po3 (SEQ ID NO: 1417) exhibits between 40 to 80% rescue of full length STMN2. In particular embodiments, QSN-173-po3 (SEQ ID NO: 1417) exhibits between 50 to 60% rescue of full length STMN2. In particular embodiments, QSN-173-po5 (SEQ ID NO: 1418) exhibits between 30 to 100% rescue of full length STMN2. In particular embodiments, QSN-173-po5 (SEQ ID NO: 1418) exhibits between 40 to 80% rescue of full length STMN2. In particular embodiments, QSN-173-po5 (SEQ ID NO: 1418) exhibits between 50 to 60% rescue of full length STMN2.

In particular embodiments, QSN-144-po3 (SEQ ID NO: 1419) exhibits between 30 to 100% rescue of full length STMN2. In particular embodiments, QSN-144-po3 (SEQ ID NO: 1419) exhibits between 40 to 80% rescue of full length STMN2. In particular embodiments, QSN-144-po3 (SEQ ID NO: 1419) exhibits between 50 to 60% rescue of full length STMN2. In particular embodiments, QSN-144-po5 (SEQ ID NO: 1420) exhibits between 30 to 100% rescue of full length STMN2. In particular embodiments, QSN-144-po5 (SEQ ID NO: 1420) exhibits between 40 to 80% rescue of full length STMN2. In particular embodiments, QSN-144-po5 (SEQ ID NO: 1420) exhibits between 50 to 60% rescue of full length STMN2.

In particular embodiments, QSN-185-po3 (SEQ ID NO: 1421) exhibits between 30 to 100% rescue of full length STMN2. In particular embodiments, QSN-185-po3 (SEQ ID NO: 1421) exhibits between 40 to 80% rescue of full length STMN2. In particular embodiments, QSN-185-po3 (SEQ ID NO: 1421) exhibits between 50 to 60% rescue of full length STMN2. In particular embodiments, QSN-185-po5 (SEQ ID NO: 1422) exhibits between 30 to 100% rescue of full length STMN2. In particular embodiments, QSN-185-po5 (SEQ ID NO: 1422) exhibits between 40 to 80% rescue of full length STMN2. In particular embodiments, QSN-185-po5 (SEQ ID NO: 1422) exhibits between 50 to 60% rescue of full length STMN2.

In particular embodiments, QSN-237-po3 (SEQ ID NO: 1423) exhibits between 30 to 100% rescue of full length STMN2. In particular embodiments, QSN-237-po3 (SEQ ID NO: 1423) exhibits between 40 to 80% rescue of full length STMN2. In particular embodiments, QSN-237-po3 (SEQ ID NO: 1423) exhibits between 50 to 60% rescue of full length STMN2. In particular embodiments, QSN-237-po5 (SEQ ID NO: 1424) exhibits between 30 to 100% rescue of full length STMN2. In particular embodiments, QSN-237-po5 (SEQ ID NO: 1424) exhibits between 40 to 80% rescue of full length STMN2. In particular embodiments, QSN-237-po5 (SEQ ID NO: 1424) exhibits between 50 to 60% rescue of full length STMN2.

### Additional Chemically Modified STMN2 Antisense Oligonucleotides

STMN2 AONs described herein, can include chemically modified nucleosides, including modified ribonucleosides and modified deoxyribonucleosides. Chemically modified nucleosides include, but are not limited to, uracil, uracine, uridine, 2′-O-(2-methoxyethyl) modifications, for example, 2′-O-(2-methoxyethyl)guanosine, 2′-O-(2-methoxyethyl)adenosine, 2′-O-(2-methoxyethyl)cytosine, and 2′-O-(2-methoxyethyl)thymidine. In certain embodiments, mixed modalities, e.g., a combination of a STMN2 peptide nucleic acid (PNA) and a STMN2 locked nucleic acid (LNA). Chemically modified nucleosides also include, but are not limited to, locked nucleic acids (LNAs), 2′-MOE, 2′-O-methyl, 2′-fluoro, and 2′-fluoro-β-D-arabinonucleotide (FANA), and Fluoro Cyclohexenyl nucleic acid (F-CeNA) modifications. Chemically modified nucleosides that can be included in STMN2 AONs described herein are described in Johannes and Lucchino, (2018) “Current Challenges in Delivery and Cytosolic Translocation of Therapeutic RNAs” Nucleic Acid Ther. 28(3): 178-93; Rettig and Behlke, (2012) “Progress toward in vivo use of siRNAs-II” Mol Ther 20:483-512; and Khvorova and Watts, (2017) “The chemical evolution of oligonucleotide therapies of clinical utility” Nat Biotechnol., 35(3):238-48, the contents of each of which are incorporated by reference herein.

STMN2 AONs described herein can include chemical modifications that promote stabilization of an oligonucleotide's terminal 5′-phosphate and phosphatase-resistant analogs of 5′-phosphate. Chemical modifications that promote oligonucleotide terminal 5′-phosphate stabilization or which are phosphatase-resistant analogs of 5′-phosphate include, but are not limited to, 5′-methyl phosphonate, 5′-methylenephosphonate, 5′-methylenephosphonate analogs, 5′-E-vinyl phosphonate (5′-E-VP), 5′-phosphorothioate, and 5′-C-methyl analogs. Chemical modifications that promote AON terminal 5′-phosphate stabilization and phosphatase-resistant analogues of 5′-phosphate are described in Khvorova and Watts, (2017) “The chemical evolution of oligonucleotide therapies of clinical utility” Nat Biotechnol., 35(3):238-48, the contents of which are incorporated by reference herein.

In some embodiments described herein, STMN2 AONs described herein can include chemically modified nucleosides, for example, 2′ O-methyl ribonucleosides, for example, 2′ O-methyl cytidine, 2′ O-methyl guanosine, 2′ O-methyl uridine, and/or 2′ O-methyl adenosine. STMN2 AONs described herein can include one or more chemically modified bases, including a 5-methylpyrimidine, for example, 5-methyl cytosine, and/or a 5-methylpurine, for example, 5-methylguanine. Chemically modified bases can further include pseudo-uridine or 5′methoxyuridine. STMN2 AONs described herein can include any of the following chemically modified nucleosides: 5-methyl-2′-O-methylcytidine, 5-methyl-2′-O-methylthymidine, 5-methylcytidine, 5-methyluridine, and/or 5-methyl 2′-deoxycytidine.

STMN2 AONs described herein can include a phosphate backbone where one or more of the oligonucleoside linkages is a phosphate linkage. STMN2 AONs described herein may include a modified oligonucleotide backbone, where one or more of the nucleoside linkages of the nucleobase sequence is selected from the group consisting of a phosphorothioate linkage, an alkyl phosphate linkage, a phosphorodithioate linkage, a phosphotriester linkage, an alkylphosphonate linkage, a 3-methoxypropyl phosphonate linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage. In some embodiments of STMN2 AONs described herein, at least one internucleoside linkage of the nucleobase sequence is a phosphorothioate linkage. For example, in some embodiments of STMN2 AONs described herein, one, two, three, or more internucleoside linkages of the nucleobase sequence is a phosphorothioate linkage. In preferred embodiments of STMN2 AONs described herein, all internucleoside linkages of the nucleobase sequence are phosphorothioate linkages. Thus, in some embodiments, all of the nucleotide linkages of a STMN2 AON of any of SEQ ID NOs: 1-446, SEQ ID NOs: 894-918, SEQ ID NOs: 945-1390, or SEQ ID NOs: 1392-1432 are phosphorothioate linkages. In some embodiments, one or more of the nucleotide linkages of a STMN2 AON of any of SEQ ID NOs: SEQ ID NOs: 1-446, SEQ ID NOs: 894-918, SEQ ID NOs: 945-1390, or SEQ ID NOs: 1392-1432 are phosphorothioate linkages.

It is contemplated that in some embodiments, a disclosed STMN2 AON may optionally have at least one modified nucleobase, e.g., 5-methyl cytosine, and/or at least one methylphosphonate nucleotide, which is placed, for example, either at only one of the 5′ or 3′ ends or at both 5′ and 3′ ends or along the oligonucleotide sequence. In some embodiments, all internucleoside linkages of a STMN2 AON oligonucleotide of the present disclosure are phosphorothioate linkages, and each of the linked nucleosides of the oligonucleotide are 2′-O-(2-methoxyethyl) (2′-MOE) nucleosides and each “C” is replaced with a 5-MeC.

Contemplated STMN2 AONs may optionally include at least one modified sugar. For example, the sugar moiety of at least one nucleotide constituting the oligonucleotide is a ribose in which the 2′-OH group may be replaced by any one selected from the group consisting of OR, R, R′OR, SH, SR, NH2, NR₂, N₃, CN, F, Cl, Br, and I (wherein R is an alkyl or aryl and R′ is an alkylene). Examples of a modified sugar moiety include a 2′-OMe modified sugar moiety, bicyclic sugar moiety, 2′ -O-(2-methoxyethyl) (2′MOE), 2′-deoxy-2′-fluoro nucleoside, 2′ -fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

In some embodiments, STMN2 AONs comprise 2′OMe (e.g., an STMN2 AON comprising one or more 2′OMe modified sugar), MOE (e.g., an STMN2 AON comprising one or more MOE modified sugar (e.g., 2′-MOE)), PNA (e.g., a STMN2 AON comprising one or more N-(2-aminoethyl)-glycine units linked by amide bonds or carbonyl methylene linkage as repeating units in place of a sugar-phosphate backbone), LNA (e.g., a STMN2 AON comprising one or more locked ribose, and can be a mixture of 2′-deoxy nucleotides or 2′OMe nucleotides), c-ET (e.g., a STMN2 AON comprising one or more cET sugar), cMOE (e.g., a STMN2 AON comprising one or more cMOE sugar), morpholino oligomer (e.g., a STMN2 AON comprising a backbone comprising one or more PMO), deoxy-2′-fluoro nucleoside (e.g., a STMN2 AON comprising one or more 2′-fluoro-β-D-arabinonucleoside), ENA (e.g., a STMN2 AON comprising one or more ENA modified sugar), HNA (e.g., a STMN2 AON comprising one or more HNA modified sugar), or tcDNA (e.g., a STMN2 AON comprising one or more tcDNA modified sugar). In some embodiments, a STMN2 AON comprises one or more phosphorothioate linkage, phosphodiester linkage, phosphotriester linkage, methylphosphonate linkage, phosphoramidate linkage, morpholino linkage, PNA linkage, or any combination of phosphorothioate linkage, phosphodiester linkage, a phosphotriester linkage, methylphosphonate linkage, phosphoramidate linkage, morpholino linkage, and PNA linkage. In some embodiments, a STMN2 AON comprises one or more phosphorothioate linkage, phosphodiester linkage, or a combination of phosphorothioate and phosphodiester linkages.

### Motor Neuron Diseases

Motor neuron diseases are a group of diseases characterized by loss of function of motor neurons that coordinate voluntary movement of muscles by the brain. Motor neuron diseases may affect upper and/or lower motor neurons, and may have sporadic or familial origins. Motor neuron diseases include amyotrophic lateral sclerosis (ALS or Lou Gehrig's disease), progressive bulbar palsy, pseudobulbar palsy, progressive muscular atrophy, primary lateral sclerosis, spinal muscular atrophy, post-polio syndrome, and ALS with frontotemporal dementia.

Symptoms of motor neuron diseases include muscle decay or weakening, muscle pain, spasms, slurred speech, difficulty swallowing, loss of muscle control, joint pain, stiff limbs, difficulty breathing, drooling, and complete loss of muscle control, including over basic functions such as breathing, swallowing, eating, speaking, and limb movement. These symptoms are also sometimes accompanied by depression, loss of memory, difficulty with planning, language deficits, altered behavior, and difficulty assessing spatial relationships and/or changes in personality.

Motor neuron diseases can be assessed and diagnosed by a clinician of skill, for example, a neurologist, using various tools and tests. For example, the presence or risk of developing a motor neuron disease can be assessed or diagnosed using blood and urine tests (for example, tests that assay for the presence of creatinine kinase), magnetic resonance imaging (MRI), electromyography (EMG), nerve conduction study (NCS), spinal tap, lumbar puncture, and/or muscle biopsy. Motor neuron diseases can be diagnosed with the aid of a physical exam and/or a neurological exam to assess motor and sensory skills, nerve function, hearing and speech, vision, coordination and balance, mental status, and changes in mood or behavior.

### Amyotrophic Lateral Sclerosis

ALS is a progressive motor neuron disease that disrupts signals to all voluntary muscles. ALS results in atrophy of both upper and lower motor neurons. Symptoms of ALS include weakening and wasting of the bulbar muscles, general and bilateral loss of strength, spasticity, muscle spasms, muscle cramps, fasciculations, slurred speech, and difficulty breathing or loss of ability to breathe. Some individuals with ALS also suffer from cognitive decline. At the molecular level, ALS is characterized by protein and RNA aggregates in the cytoplasm of motor neurons, including aggregates of the RNA-binding protein TDP43.

ALS is most common in males above 40 years of age, although it can also occur in women and children. Risk of ALS is also heightened in individuals who smoke, are exposed to chemicals such as lead, or who have served in the military. Most instances of ALS are sporadic, while only about 10% of cases are familial. Causes of ALS include sporadic or inherited genetic mutations, high levels of glutamate, protein mishandling. Genetic mutations associated with ALS include mutations in the genes SOD1, C9orf72, TARDBP, FUS, ANG, ATXN2, CHCHD10, CHMP2B, DCTN1, ErbB4, FIG4, HNRPA1, MATR3, NEFH, OPTN, PFN1, PRPH, SETX, SIGMAR1, SMN1, SPG11, SQSTM1, TBK1, TRPM7, TUBA4A, UBQLN2, VAPB, and VCP.

### Frontotemporal Dementia

Frontotemporal dementia (FTD) is a form of dementia that affects the frontal and temporal lobes of the brain. It has an earlier average age of onset than Alzheimer's disease—40 years of age. Symptoms of FTD include extreme changes in behavior and personality, speech and language problems, and movement-related symptoms such as tremor, rigidity, muscle spasm, weakness, and difficulty swallowing. Subtypes of FTD include behavior variant frontotemporal dementia (bvFTD), characterized by changes in personality and behavior, and primary progressive aphasia (PPA), which affects language skills, speaking, writing and comprehension. FTD is associated with tau protein accumulation (Pick bodies) and altered TDP43 function. About 30% of cases of FTD are familial, and no other risk factors other than family history of the disease are known. Genetic mutations associated with FTD include mutations in the genes C9orf72, Progranulin (GRN), microtubule-associated protein tau (MAPT), UBQLN2, VPC, CHMP2B, TARDBP, FUS, ITM2B, CHCHD10, SQSTM1, PSEN1, PSEN2, CTSF, CYP27A1, TBK1 and TBP.

Amyotrophic Lateral Sclerosis with Frontotemporal Dementia

Amyotrophic lateral sclerosis with frontotemporal dementia (ALS with FTD) is a clinical syndrome in which FTD and ALS occur in the same individual. Interestingly, mutations in C9orf72 are the most common cause of familial forms of ALS and/or FTD. Additionally, mutations in TBK1, VCP, SQSTMI, UBQLN2 and CHMP2B are also associated with ALS with FTD. Symptoms of ALS with FTD include dramatic changes in personality, as well as muscle weakness, muscle atrophy, fasciculations, spasticity, dysarthria, dysphagia, and degeneration of the spinal cord, motor neurons, and frontal and temporal lobes of the brain. At the molecular level, ALS with FTD is characterized by the accumulation of TDP-43 and/or FUS proteins in the cytoplasm. TBK1 mutations are associated with ALS, FTD, and ALS with FTD.

### Methods of Treatment

The disclosure contemplates, in part, treating neurological diseases (for example, amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, corticobasal degeneration (CBD) and/or neuropathies such a chemotherapy induced neuropathy in a patient in need thereof comprising administering a disclosed inhibitor of STMN2 transcripts that include a cryptic exon, for example, a STMN2 AON. In some embodiments, provided herein are methods for treatment of a neurological disease in a patient in need thereof, comprising administering a disclosed STMN2 AON. In some embodiments of the disclosure, an effective amount of a disclosed inhibitor of STMN2 transcripts that include a cryptic exon may be administered to a patient in need thereof to treat a neurological disease, and/or to increase, restore, or stabilize expression of STMN2 mRNA that is capable of translation to produce a functional STMN2 protein, thereby increase, restore, or stabilize STMN2 activity and/or function.

In some embodiments, treating a neurological disease comprises at least ameliorating or reducing one symptom associated with the neurological disease (for example, reducing muscle weakness in a patient with ALS). Methods of treating a neurological disease (for example, ALS, FTD, or ALS with FTD) in a patient suffering therefrom are provided, that include administering a disclosed inhibitor of STMN2 transcripts that include a cryptic exon, for example, a STMN2 AON. In some embodiments, methods of slowing the progression of a neurological disease, for example, a motor neuron disease, are provided.

Provided herein are methods of treating, reducing the risk of developing, or delaying the onset of a neurological disease in a subject in need thereof comprising administering a disclosed inhibitor of STMN2 transcripts that include a cryptic exon, for example, a STMN2 AON. The methods include for example, treating a subject at risk of developing a neurological disease; e.g., administering to the subject an effective amount of a disclosed STMN2 AON. Neurological diseases that can be treated in this manner include motor neuron diseases, ALS, FTD, ALS with FTD, progressive bulbar palsy, pseudobulbar palsy, progressive muscular atrophy, primary lateral sclerosis, spinal muscular atrophy, and post-polio syndrome.

Methods of preventing or treating neurological diseases (for example, PD, ALS, FTD, and ALS with FTD) form part of this disclosure. Such methods may comprise administering to a patient in need thereof or a patient at risk, a pharmaceutical preparation comprising a STMN2 AON such as a STMN2 AON disclosed herein. For example, a method of preventing or treating a neurological disease is provided comprising administering to a patient in need thereof a STMN2 AON disclosed herein.

Patients treated using an above method may experience an increase, restoration of, or stabilization of STMN2 mRNA expression, which is capable of translation to produce a functional STMN2 protein, of at least about 5%, 10%, 20%, 30%, 40% or even 50%, thereby increase, restore, or stabilize STMN2 activity and/or function in a target cell (for example, a motor neuron) after administering an inhibitor of STMN2 transcripts that include a cryptic exon, after e.g. 1 day, 2 days, 1 week, 2 weeks, 3 weeks, 4 weeks, 5 weeks, 6 weeks, 7 weeks, 8 weeks, 1 month, 2 months, 3, months, 4 months, 5, months, or 6 months or more. Administering such inhibitor of STMN2 transcripts that include a cryptic exon may be on, e.g., at least a daily basis. The inhibitor of STMN2 transcripts that include a cryptic exon may be administered orally. In some embodiments, the inhibitor of STMN2 transcripts that include a cryptic exon is administered intrathecally or intracisternally. For example, in an embodiment described herein, an inhibitor of STMN2 transcripts that include a cryptic exon is administered intrathecally or intracisternally about every 3 months. The delay or amelioration of clinical manifestation of a neurological disease in a patient as a consequence of administering an inhibitor of STMN2 transcripts that include a cryptic exon disclosed here may be at least e.g., 6 months, 1 year, 18 months or even 2 years or more as compared to a patient who is not administered an inhibitor of STMN2 transcripts that include a cryptic exon, such as one disclosed herein.

The inhibitors of STMN2 transcripts that include a cryptic exon, for example STMN2 AONs, of the invention can be used alone or in combination with each other whereby at least two inhibitors of STMN2 transcripts that include a cryptic exon of the invention are used together in a single composition or as part of a treatment regimen. STMN2 oligonucleotides can be used alone or in combination with each other whereby at least two STMN2 oligonucleotides are used together in a single composition or as part of a treatment regimen. The inhibitors of STMN2 transcripts that include a cryptic exon of the invention may also be used in combination with other drugs for treating neurological diseases or conditions.

### Treatment and Evaluation

A patient, as described herein, refers to any animal at risk for, suffering from or diagnosed with a neurological disease, including, but not limited to, mammals, primates, and humans. In certain embodiments, the patient may be a non-human mammal such as, for example, a cat, a dog, or a horse. In certain embodiments, the patient is a human. A patient may be an individual diagnosed with a high risk of developing a neurological disease, someone who has been diagnosed with a neurological disease, someone who previously suffered from a neurological disease, or an individual evaluated for symptoms or indications of a neurological disease, for example, any of the signs or symptoms associated with neurological diseases such as amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, corticobasal degeneration (CBD) and/or neuropathies such a chemotherapy induced neuropathy.

“A patient in need,” as used herein, refers to a patient suffering from any of the symptoms or manifestations of a neurological disease, a patient who may suffer from any of the symptoms or manifestations of a neurological disease, or any patient who might benefit from a method of the disclosure for treating a neurological disease. A patient in need may include a patient who is diagnosed with a risk of developing a neurological disease, a patient who has suffered from a neurological disease in the past, or a patient who has previously been treated for a neurological disease.

“Effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of a disclosed inhibitor of STMN2 transcripts that include a cryptic exon is the amount of the inhibitor of STMN2 transcripts that include a cryptic exon necessary to treat a neurological disease in a patient such that administration of the agent prevents a neurological disease from occurring in a subject, prevents neurological disease progression (e.g., prevents the onset or increased severity of symptoms of the neurological such as muscle weakening, spasms, or fasciculation), or relieves or completely ameliorates all associated symptoms of a neurological disease, i.e. causes regression of the disease.

Efficacy of treatment may be evaluated by means of evaluation of gross symptoms associated with a neurological disease, analysis of tissue histology, biochemical assay, imaging methods such as, for example, magnetic resonance imaging, or other known methods. For instance, efficacy of treatment may be evaluated by analyzing gross symptoms of the disease such as changes in muscle strength and control or other aspects of gross pathology associated with a neurological disease following administration of a disclosed inhibitor of STMN2 transcripts that include a cryptic exon to a patient suffering from a neurological disease.

Efficacy of treatment may also be evaluated at the tissue or cellular level, for example, by means of obtaining a tissue biopsy (e.g., a brain, spinal, muscle, or motor neuron tissue biopsy) and evaluating gross tissue or cell morphology or staining properties. Biochemical assays that examine protein or RNA expression may also be used to evaluate efficacy of treatment. For instance, one may evaluate levels of a protein or gene product indicative of a neurological disease, in dissociated cells or non-dissociated tissue via immunocytochemical, immunohistochemical, Western blotting, or Northern blotting methods, or methods useful for evaluating RNA levels such as quantitative or semi-quantitative polymerase chain (e.g., digital PCR (DigitalPCR, dPCR, or dePCR), qPCR etc.) reaction. One may also evaluate the presence or level of expression of useful biomarkers (e.g., neurofilament light (NEFL), neurofilament heavy (NEFH), TDP-43 or p75 extracellular domain (^{p75ECD})) found in spinal cord fluid, cerebrospinal fluid, extracellular vesicles (for example, exosome-like cerebrospinal fluid extracellular vesicles (“CSF exosomes”), such as those described in Welton et al., (2017) “Cerebrospinal fluid extracellular vesicle enrichment for protein biomarker discovery in neurological disease; multiple sclerosis” J Extracell Vesicles., 6(1):1-10; and Street et al., (2012) “Identification and proteomic profiling of exosomes in human cerebrospinal fluid” J Transl. Med., 10:5), urine, fecal matter, lymphatic fluid, blood, plasma, or serum to evaluate disease state and efficacy of treatment. One may also evaluate the presence or level of expression of useful biomarkers found in the plasma, neuronal extracellular vesicles/exosomes. Additional measurements of efficacy may include strength duration time constant (SDTC), short interval cortical inhibition (SICI), dynamometry, accurate test of limb isometric strength (ATLIS), compound muscle action potential (bio), and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in C9ORF72-associated amyotrophic lateral sclerosis (c9ALS) patients. CSF pNFH as a prognostic biomarker for clinical trials, which will increase the likelihood of successfully developing a treatment for c9ALS.

In evaluating efficacy of treatment, suitable controls may be chosen to ensure a valid assessment. For instance, one can compare symptoms evaluated in a patient with a neurological disease following administration of a disclosed inhibitor of STMN2 transcripts that include a cryptic exon to those symptoms in the same patient prior to treatment or at an earlier point in the course of treatment or in another patient not diagnosed with the neurological disease. Alternatively, one may compare the results of biochemical or histological analysis of tissue following administration of a disclosed inhibitor of STMN2 transcripts that include a cryptic exon with those of tissue from the same patient or from an individual not diagnosed with the neurological disease or from the same patient prior to administration of the inhibitor of STMN2 transcripts that include a cryptic exon. Additionally, one may compare blood, plasma, serum, cell, urine, lymphatic fluid, spinal cord fluid, cerebrospinal fluid, or fecal samples following administration of the inhibitor of STMN2 transcripts that include a cryptic exon with comparable samples from an individual not diagnosed with the neurological disease or from the same patient prior to administration of the inhibitor of STMN2 transcripts that include a cryptic exon. In some embodiments one may compare extracellular vesicles (for example CSF exosomes), following administration of the inhibitor of STMN2 transcripts that include a cryptic exon with extracellular vesicles from an individual not diagnosed with the neurological disease or from the same patient prior to administration of the inhibitor of STMN2 transcripts that include a cryptic exon.

Validation of inhibition of STMN2 transcripts that include a cryptic exon may be determined by direct or indirect assessment of STMN2 expression levels or activity. For instance, biochemical assays that measure STMN2 protein or RNA expression may be used to evaluate overall inhibition of STMN2 transcripts that include a cryptic exon. For instance, one may measure STMN2 protein levels in cells or tissue by Western blot to evaluate overall STMN2 levels. One may also measure STMN2 mRNA levels by means of Northern blot or quantitative polymerase chain reaction to determine overall inhibition of STMN2 transcripts that include a cryptic exon. One may also evaluate STMN2 protein levels or levels of another protein indicative of STMN2 signaling activity in dissociated cells, non-dissociated tissue, extracellular vesicles (for example, CSF exosomes), blood, serum, or fecal matter via immunocytochemical or immunohistochemical methods.

Modulation of splicing of STMN2 transcripts that include a cryptic exon may also be evaluated indirectly by measuring parameters such as autophagy, endocytosis, protein aggregation, and the presence or level of expression of useful biomarkers (e.g., neurofilament light (NEFL), neurofilament heavy (NEFH), TDP-43, or p75^{ECD} found in plasma, spinal cord fluid, cerebrospinal fluid, extracellular vesicles (for example, CSF exosomes), blood, urine, lymphatic fluid, fecal matter, or tissue to evaluate efficacy of inhibition of STMN2 transcripts that include a cryptic exon. Inhibition of STMN2 transcripts that include a cryptic exon may also be evaluated indirectly by measuring parameters such as autophagy, endocytosis, protein aggregation, and the presence or level of expression of physiological biomarkers such as compound muscle action potential (bio). Additional measurements may include strength duration time constant (SDTC), short interval cortical inhibition (SICI), dynamometry, accurate test of limb isometric strength (ATLIS), compound muscle action potential, and ALSFRS-R. In certain embodiments, urinary neurotrophin receptor p75 extracellular domain (p75^{ECD}) is a disease progression and prognostic biomarker in amyotrophic lateral sclerosis (ALS). Phosphorylated neurofilament heavy chain (pNFH) in cerebrospinal fluid (CSF) predict disease status and survival in c9ALS patients. CSF pNFH as a prognostic biomarker for clinical trials, which will increase the likelihood of successfully developing a treatment for c9ALS.

In some embodiments, the present disclosure provides methods of correcting splicing of a STMN2 transcript with a cryptic exon, and thereby restoring full length STMN2 protein expression in cells of a patient suffering from a neurological disease. Splicing of a STMN2 transcript may be corrected in any cell in which STMN2 expression or activity occurs, including cells of the nervous system (including the central nervous system, the peripheral nervous system, motor neurons, the brain, the brain stem, the frontal lobes, the temporal lobes, the spinal cord), the musculoskeletal system, spinal fluid, and cerebrospinal fluid. Cells of the musculoskeletal system include skeletal muscle cells (e.g., myocytes). Motor neurons include upper motor neurons and lower motor neurons.

### Pharmaceutical Compositions and Routes of Administration

The present disclosure also provides methods for treating a neurological disease via administration of a pharmaceutical composition comprising a disclosed inhibitor of STMN2 transcripts that include a cryptic exon. In another aspect, the disclosure provides a pharmaceutical composition for use in treating a neurological disease. The pharmaceutical composition may be comprised of a disclosed antisense oligonucleotide that targets STMN2 transcripts that include a cryptic exon, and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat a neurological disease. In some embodiments, contemplated herein are pharmaceutical compositions comprising a disclosed inhibitor of STMN2 transcripts that include a cryptic exon, and a pharmaceutically acceptable carrier. In another aspect, the disclosure provides use of a disclosed inhibitor of STMN2 transcripts that include a cryptic exon in the manufacture of a medicament for treating a neurological disease. “Medicament,” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In one embodiment, a disclosed inhibitor of STMN2 transcripts that include a cryptic exon and any pharmaceutical composition thereof may be administered by one or several routes, including topically, intrathecally, intracisternally, parenterally (e.g., subcutaneous, intramuscular, intradermal, intraduodenal, or intravenous), intralesionally, orally, rectally, buccally, sublingually, vaginally, pulmonarily, intratracheally, intranasally, transdermally, or intraduodenally. The term parenteral as used herein includes subcutaneous injections, intrapancreatic administration, intravenous, intracisternal, intrathecal, intramuscular, intraperitoneal, intrasternal injection or infusion techniques. For example, a disclosed inhibitor of STMN2 transcripts that include a cryptic exon may be administered subcutaneously to a subject. In another example, a disclosed inhibitor of STMN2 transcripts that include a cryptic exon may be administered orally to a subject. In another example, a disclosed inhibitor of STMN2 transcripts that include a cryptic exon may be administered directly to the nervous system, or specific regions or cells of the nervous system (e.g., the brain, brain stem, lower motor neurons, spinal cord, upper motor neurons) via parenteral administration, for example, a disclosed inhibitor of STMN2 transcripts that include a cryptic exon may be administered intrathecally or intracisternally.

In some embodiments, an inhibitor of STMN2 transcripts that include a cryptic exon, for example a STMN2 AON, can be encapsulated in a nanoparticle coating. It is believed that nanoparticle encapsulation prevents AON degradation and enhances cellular uptake. For example, in some embodiments an inhibitor of STMN2 transcripts that include a cryptic exon is encapsulated in a coating of a cationic polymer, for example, a synthetic polymer (e.g., poly-L-lysine, polyamidoamine, a poly(β-amino ester), and polyethyleneimine) or a naturally occurring polymer (e.g., chitosan and a protamine). In some embodiments, an inhibitor of STMN2 transcripts that include a cryptic exon is encapsulated in a lipid or lipid-like material, for example, a cationic lipid, a cationic lipid-like material, or an ionizable lipid that is positively charged only at an acidic pH. For example, in some embodiments, an inhibitor of STMN2 transcripts that include a cryptic exon is encapsulated in a lipid nanoparticle that includes hydrophobic moieties, e.g., cholesterol and/or a polyethylene glycol (PEG) lipid.

In some embodiments, an inhibitor of STMN2 transcripts that include a cryptic exon, for example, a STMN2 AON, is conjugated to a bioactive ligand. For example, in some embodiments described herein, an inhibitor of STMN2 transcripts that include a cryptic exon such as a STMN2 AON is conjugated to a peptide, a lipid, N-acetylgalactosamine (GalNAc), cholesterol, vitamin E, an antibody, or a cell-penetrating peptide (for example, transactivator of transcription (TAT) and penetratine).

Pharmaceutical compositions containing a disclosed inhibitor of STMN2 transcripts that include a cryptic exon, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations, in some embodiments, are sterile. Sterilization can be accomplished, for example, by filtration through sterile filtration membranes. Where the composition is lyophilized, filter sterilization can be conducted prior to or following lyophilization and reconstitution.

### Parenteral Administration

The pharmaceutical compositions of the disclosure can be formulated for parenteral administration, e.g., formulated for injection via the intravenous, intracisternal, intramuscular, subcutaneous, intrathecal, intralesional, or intraperitoneal routes. The preparation of an aqueous composition, such as an aqueous pharmaceutical composition containing a disclosed inhibitor of STMN2 transcripts that include a cryptic exon, will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

The pharmaceutical forms suitable for injectable use include sterile aqueous solutions or dispersions; formulations including normal saline, phosphate buffer saline, artificial cerebrospinal fluid, sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, a disclosed STMN2 antisense oligonucleotide may be suspended in a carrier fluid comprising 1% (w/v) sodium carboxymethylcellulose and 0.1% (v/v) TWEEN™80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the disclosure may be prepared by incorporating a disclosed STMN2 antisense oligonucleotide (e.g., inhibitor of STMN2 transcripts that include a cryptic exon) in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the disclosed inhibitor of STMN2 transcripts that include a cryptic exon to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and for example, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose , petrolatum, polyethylene glycol, polyvinyl alcohol, polyvinylpyrrolidone, carboxymethylcellulose and the like.

### Oral Administration

In some embodiments, contemplated herein are compositions suitable for oral delivery of a disclosed inhibitor of STMN2 transcripts that include a cryptic exon, e.g., tablets that include an enteric coating, e.g., a gastro-resistant coating, such that the compositions may deliver an inhibitor of STMN2 transcripts that include a cryptic exon to, e.g., the gastrointestinal tract of a patient.

For example, a tablet for oral administration is provided that comprises granules (e.g., is at least partially formed from granules) that include a disclosed inhibitor of STMN2 transcripts that include a cryptic exon, e.g., a STMN2 antisense oligonucleotide, e.g., a STMN2 antisense oligonucleotide represented by any of SEQ ID NOs: 1-446, SEQ ID NOs: 894-918, SEQ ID NOs: 945-1390, or SEQ ID NOs: 1392-1432, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a disclosed inhibitor of STMN2 transcripts that include a cryptic exon, e.g. a STMN2 antisense oligonucleotide, e.g., a STMN2 antisense oligonucleotide represented by any of SEQ ID NOs: 1-446, SEQ ID NOs: 894-918, SEQ ID NOs: 945-1390, or SEQ ID NOs: 1392-1432, and a pharmaceutically acceptable salt, e.g., a STMN2 antisense oligonucleotide, e.g., an antisense oligonucleotide represented by any of SEQ ID NOs: 1-446, SEQ ID NOs: 894-918, SEQ ID NOs: 945-1390, or SEQ ID NOs: 1392-1432, and a pharmaceutically acceptable filler. For example, a disclosed inhibitor of STMN2 transcripts that include a cryptic exon and a filler may be blended together, optionally, with other excipients, and formed into granules. In some embodiments, the intragranular phase may be formed using wet granulation, e.g., a liquid (e.g., water) is added to the blended inhibitor of STMN2 transcripts that include a cryptic exon compound and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pre-gelatinized starch, calcium carbonate, and others including combinations thereof.

In some embodiments, a disclosed formulation may include an intragranular phase and/or an extragranular phase that includes a binder, which may generally function to hold the ingredients of the pharmaceutical formulation together. Exemplary binders of the disclosure may include, but are not limited to, the following: starches, sugars, cellulose or modified cellulose such as hydroxypropyl cellulose, lactose, pre-gelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as, but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising a disclosed inhibitor of STMN2 transcripts that include a cryptic exon and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, partially hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE®, Eudragit® grades. In some embodiments, an enteric coating may comprise about 5% to about 10%, about 5% to about 20%, 8% to about 15%, about 8% to about 20%, about 10% to about 20%, or about 12 to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g., about 0.5% to about 10%, or about 1% to about 20%, by weight of a disclosed STMN2 antisense oligonucleotide or a pharmaceutically acceptable salt thereof. Such a tablet may include for example, about 0.5% to about 60% by weight of mannitol, e.g., about 30% to about 50% by weight mannitol, e.g., about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of a disclosed STMN2 antisense oligonucleotide, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g., about 2% to about 4% sodium starch glycolate by weight.

In another contemplated embodiment, a pharmaceutical tablet formulation for oral administration of a disclosed inhibitor of STMN2 transcripts that include a cryptic exon comprises an intra-granular phase, wherein the intra-granular phase includes a disclosed STMN2 AON or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 20% by weight of the tablet. For example, a pharmaceutically acceptable tablet for oral use may comprise about .5% to 10% by weight of a disclosed STMN2 AON, e.g., a disclosed STMN2 AON or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of a disclosed STMN2 AON, e.g., a disclosed STMN2 AON or a pharmaceutically acceptable salt thereof, about 40% by weight mannitol, about 8% by weight microcrystalline cellulose, about 5% by weight hydroxypropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO 2010/054826, which is hereby incorporated by reference in its entirety).

The rate at which the coating dissolves and the active ingredient is released is its dissolution rate. In an embodiment, a contemplated tablet may have a dissolution profile, e.g. when tested in a USP/EP Type 2 apparatus (paddle) at 100 rpm and 37° C. in a phosphate buffer with a pH of 7.2, of about 50% to about 100% of the inhibitor of STMN2 transcripts that include a cryptic exon releasing after about 120 minutes to about 240 minutes, for example after 180 minutes. In another embodiment, a contemplated tablet may have a dissolution profile, e.g. when tested in a USP/EP Type 2 apparatus (paddle) at 100 rpm and 37° C. in diluted HCl with a pH of 1.0, where substantially none of the inhibitor of STMN2 transcripts that include a cryptic exon is released after 120 minutes. A contemplated tablet, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the inhibitor of STMN2 transcripts that include a cryptic exon releasing after 30 minutes.

In some embodiments, methods provided herein may further include administering at least one other agent that is directed to treatment of diseases and disorders disclosed herein. In one embodiment, contemplated other agents may be co-administered (e.g., sequentially or simultaneously).

### Dosage and Frequency of Administration

The dosage or amounts described below refer either to the oligonucleotide or a pharmaceutically acceptable salt thereof.

In some embodiments, formulations include dosage forms that include at least 1 μg, at least 5 μg, at least 10 μg, at least 20 μg, at least 30 μg, at least 40 μg, at least 50 μg, at least 60 μg, at least 70 μg, at least 80 μg, at least 90 μg, or at least 100 μg of an inhibitor, for example, a STMN2 antisense oligonucleotide, of STMN2 transcripts that include a cryptic exon. In some embodiments, formulations include dosage forms that include from 10 mg to 500 mg, from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, from 450 mg to 500 mg, from 500 mg to 600 mg, from 600 mg to 700 mg, from 700 mg to 800 mg, from 800 mg to 900 mg, from 900 mg to 1 g, from 1 mg to 50 mg, from 20 mg to 40 mg, or from 1 mg to 500 mg of a STMN2 antisense oligonucleotide.

In some embodiments, formulations include dosage forms that include or consist essentially of about 10 mg to about 500 mg of an inhibitor of STMN2 transcripts that include a cryptic exon, for example, a STMN2 AON. For example, formulations that include about 10 mg, 15 mg, 20 mg, 25 mg, 30 mg, 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 250 mg, 300 mg, 350 mg, 400 mg, 450 mg, 500 mg, 600 mg, 700 mg, 800 mg, 900 mg, 1 g, 1.5 g, 2.0 g, 2.5 g, 3.0 g, 3.5 g, 4.0 g, 4.5 g, or 5.0 g of a disclosed inhibitor of STMN2 transcripts that include a cryptic exon are contemplated herein. In some embodiments, a formulation may include about 40 mg, 80 mg, or 160 mg of a disclosed inhibitor of STMN2 transcripts that include a cryptic exon. In some embodiments, a formulation may include at least 100 μg of a disclosed inhibitor of STMN2 transcripts that include a cryptic exon. For example, formulations may include about 0.1 mg, 0.2 mg, 0.3 mg, 0.4 mg, 0.5 mg, 1 mg, 5 mg, 10 mg, 15 mg, 20 mg, 25 mg, or 30 mg of a disclosed inhibitor of STMN2 transcripts that include a cryptic exon. The amount administered will depend on variables such as the type and extent of disease or indication to be treated, the overall health and size of the patient, the in vivo potency of the inhibitor of STMN2 transcripts that include a cryptic exon, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study. Dosing frequency can vary, depending on factors such as route of administration, dosage amount and the disease being treated. Exemplary dosing frequencies are once per day, once per week and once every two weeks. In some embodiments, dosing is once per day for 7 days. In some embodiments, dosing is once every 4 weeks, once every 5 weeks, once every 6 weeks, once every 7 weeks, once every 8 weeks, once every 9 weeks, once every 10 weeks, once every 11 weeks, or once every 12 weeks. In some embodiments, dosing is once a month to every three months.

### Combination Therapies

In various embodiments, a STMN2 AON as disclosed herein can be administered in combination with one or more additional therapies. The combination therapy of the disclosed oligonucleotide and the one or more additional therapies can, in some embodiments, be synergistic in treating any of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, corticobasal degeneration (CBD) and/or neuropathies such a chemotherapy induced neuropathy.

Example additional therapies include any of Riluzole (Rilutek), Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), dual AON intrathecal administration (e.g., BIIB067, BIIB078), BLIB100, levodopa/carbidopa, dopaminergic agents (e.g., ropinirole, pramipexole, rotigotine), medroxyprogesterone, KCNQ2/KCNQ3 openers, anticonvulsants and psychostimulant agents. Additional therapies can further include breathing care, physical therapy, occupational therapy, speech therapy, and nutritional support. In various embodiments, an additional therapy can be a second antisense oligonucleotide. As an example, the second antisense oligonucleotide may target a STMN2 transcript (e.g., STMN2 pre-mRNA, mature STMN2 mRNA) to modulate the expression levels of full length STMN2 protein.

In various embodiments, the disclosed oligonucleotide and the one or more additional therapies can be conjugated to one another and provided in a conjugated form. Further description regarding conjugates involving the disclosed oligonucleotide is described below. In various embodiments, the disclosed oligonucleotide and one or more additional therapies are provided concurrently. In various embodiments, the disclosed oligonucleotide and one or more additional therapies are provided simultaneously. In various embodiments, the disclosed oligonucleotide and one or more additional therapies are provided sequentially.

### Conjugates

In certain embodiments, provided herein are oligomeric compounds, which comprise an oligonucleotide (e.g., STMN2 oligonucleotide) and optionally one or more conjugate groups and/or terminal groups. Conjugate groups include one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### Conjugate Groups

In certain embodiments, a STMN2 AON is covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In particular embodiments, conjugate groups modify the circulation time (e.g., increase) of the oligonucleotides in the bloodstream such that increased concentrations of the oligonucleotides are delivered to the brain. In particular embodiments, conjugate groups modify the residence time (e.g., increase residence time) of the oligonucleotides in a target organ (e.g., brain) such that increased residence time of the oligonucleotides improves their performance (e.g., efficacy). In particular embodiments, conjugate groups increase the delivery of the oligonucleotide to the brain through the blood brain barrier and/or brain parenchyma (e.g., through receptor mediated transcytosis). In particular embodiments, conjugate groups enable the oligonucleotide to target a specific organ (e.g., the brain). In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac -glycerol or triethyl -ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GaINAc cluster (e.g., WO2014/179620)

### Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes. In particular embodiments, conjugate moieties are selected from a peptide, a lipid, N-acetylgalactosamine (GalNAc), cholesterol, vitamin E, lipoic acid, panthothenic acid, polyethylene glycol, an antibody (e.g., an antibody for crossing the blood brain barrier such as anti-transferrin receptor antibody), or a cell-penetrating peptide (e.g., transactivator of transcription (TAT) and penetratine).

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansyl sarcosine, 2,3,5-triiodobenzoic acid, fingolimod, flufenamic acid, folinic acid, a benzothiadiazide, chlorothiazide, a diazepine, indomethacin, a barbiturate, a cephalosporin, a sulfa drug, an antidiabetic, an antibacterial or an antibiotic.

### Conjugate Linkers

Conjugate moieties are attached to a STMN2 AON through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise 3 linker-nucleosides.

In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-5 -methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In certain embodiments, it is desirable for a conjugate group to be cleaved from the STMN2 AON. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxy adenosine.

### Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### Diagnostic Methods

The disclosure also provides a method of diagnosing a patient with a neurological disease that relies upon detecting levels of STMN2 expression signal in one or more biological samples of a patient. As used herein, the term “STMN2 expression signal” can refer to any indication of STMN2 gene expression, or gene or gene product activity. STMN2 gene products include RNA (e.g., mRNA), peptides, and proteins. Indices of STMN2 gene expression that can be assessed include, but are not limited to, STMN2 gene or chromatin state, STMN2 gene interaction with cellular components that regulate gene expression, STMN2 gene product expression levels (e.g., expression levels of STMN2 transcripts that include a cryptic exon, STMN2 protein expression levels), or interaction of STMN2 RNA or protein with transcriptional, translational, or post-translational processing machinery.

Detection of STMN2 expression signal may be accomplished through in vivo, in vitro, or ex vivo methods. In a preferred embodiment, methods of the disclosure may be carried out in vitro. Methods of detecting may involve detection in blood, serum, fecal matter, tissue, cerebrospinal fluid, spinal fluid, extracellular vesicles (for example, CSF exosomes), or cells of a patient. Detection may be achieved by measuring expression signal of STMN2 transcripts that include a cryptic exon in whole tissue, tissue explants, cell cultures, dissociated cells, cell extract, extracellular vesicles (for example, CSF exosomes), or body fluids, including blood, spinal fluid, cerebrospinal fluid, urine, lymphatic fluid, or serum. Contemplated methods of detection include assays that measure levels of STMN2 gene product expression such as Western blotting, FACS, ELISA, other quantitative binding assays, cell or tissue growth assays, Northern blots, quantitative or semi-quantitative polymerase chain reaction, dPCR, Quanterix SR-X™ Ultra-Sensitive Biomarker Detection System powered by Simoa® bead technology, medical imaging methods (e.g., MRI), or immunostaining methods (e.g., immunohistochemistry or immunocytochemistry).

### Additional Embodiments

Disclosed herein is a compound comprising an oligonucleotide comprising a nucleobase sequence at least 90% complementary to at least 10 contiguous nucleobases of a transcript comprising a sequence at least 90% identity to SEQ ID NO: 1391 or SEQ ID NO: 944, or a contiguous 15 to 50 nucleobase portion of SEQ ID NO: 1391 or SEQ ID NO: 944, wherein at least one nucleoside linkage of the nucleobase sequence is a non-natural linkage. Additionally disclosed herein is an oligonucleotide comprising a nucleobase sequence at least 90% complementary to at least 10 contiguous nucleobases of a transcript comprising a sequence at least 90% identity to SEQ ID NO: 1391 or SEQ ID NO: 944, or a contiguous 15 to 50 nucleobase portion of SEQ ID NO: 1391 or SEQ ID NO: 944, wherein at least one nucleoside linkage of the nucleobase sequence is a non-natural linkage.

In one aspect, the oligonucleotide comprises at least a contiguous 10 nucleobase sequence that shares 90% identity with an equal length portion of any one of SEQ ID NOs: 1-446, SEQ ID NOs: 894-918, SEQ ID NOs: 945-1390, or SEQ ID NOs: 1392-1432. In one aspect, the oligonucleotide comprises at least a contiguous 11, 12, 13, 14, 15, 16, or 17 nucleobase sequence that shares at least 90% identity with an equal length portion of any one of SEQ ID NOs: 1-446, SEQ ID NOs: 894-918, SEQ ID NOs: 945-1390, or SEQ ID NOs: 1392-1432. In one aspect, the oligonucleotide comprises at least a contiguous 10 nucleobase sequence that shares at least 90% identity with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344, 1339, or 1344, wherein at least one nucleoside linkage of the nucleobase sequence is a non-natural linkage. In one aspect, the oligonucleotide comprises at least a contiguous 11, 12, 13, 14, 15, 16, or 17 nucleobase sequence that shares at least 90% identity with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344.

Additionally disclosed herein is an oligonucleotide comprising a nucleobase sequence at least 90% complementary to at least a contiguous 10 nucleobase sequence of a transcript comprising at least 90% identity to SEQ ID NO: 944, or a contiguous 20 to 50 nucleobase portion thereof, wherein at least one nucleoside linkage of the nucleobase sequence is a non-natural linkage. In one aspect, the oligonucleotide comprises at least a contiguous 10 nucleobase sequence that shares 90% identity with an equal length portion of any one of SEQ ID NOs: 1-446 or SEQ ID NOs: 894-918. In one aspect, the oligonucleotide comprises at least a contiguous 11, 12, 13, 14, 15, 16, or 17 nucleobase sequence that shares at least 90% identity with an equal length portion of any one of SEQ ID NOs: 1-446 or SEQ ID NOs: 894-918. In one aspect, the oligonucleotide comprises at least a contiguous 10 nucleobase sequence that shares at least 90% identity with an equal length portion of any one of one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344, wherein at least one nucleoside linkage of the nucleobase sequence is a non-natural linkage. In one aspect, the oligonucleotide comprises at least a contiguous 11, 12, 13, 14, 15, 16, or 17 nucleobase sequence that shares at least 90% identity with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344.

Additionally disclosed herein is a stathmin-2 (STMN2) antisense oligonucleotide comprising a nucleic acid sequence that is at least 90% complementary with a continuous 10 nucleobase sequence of an STMN2 transcript comprising a cryptic exon comprising a nucleotide sequence at least 90% identical to SEQ ID NO: 447 or a continuous 20 to 50 nucleobase portion thereof, wherein at least one nucleoside linkage of the nucleotide sequence is a non-natural linkage. Additionally disclosed herein is a stathmin-2 (STMN2) antisense oligonucleotide comprising a nucleic acid sequence that shares at least 90% identity with a continuous 10 nucleobase sequence of any one of SEQ ID NOs: 1-446, wherein at least one nucleoside linkage of the nucleotide sequence is a non-natural linkage. In one aspect, the nucleic acid sequence shares at least 90% identity with a continuous 11, 12, 13, 14, 15, 16, or 17 nucleobase sequence of any one of SEQ ID NOs: 1-446.

Additionally disclosed herein is a stathmin-2 (STMN2) antisense oligonucleotide comprising a nucleic acid sequence that shares at least 90% identity with a continuous 10 nucleobase sequence of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344, wherein at least one nucleoside linkage of the nucleotide sequence is a non-natural linkage. In one aspect, the nucleic acid sequence shares at least 90% identity with a continuous 11, 12, 13, 14, 15, 16, or 17 nucleobase sequence of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344.

### Modifications in General

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry included all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool.

### EXAMPLES

The disclosure is further illustrated by the following examples. The examples are provided for illustrative purposes only, and are not to be construed as limiting the scope or content of the disclosure in any way.

### Example 1: Initial Design and Selection of STMN2 Antisense Oligonucleotides

Antisense oligonucleotides complementary to STMN2 RNA were designed and tested to identify STMN2 antisense oligonucleotides (AONs) capable of acting as inhibitors of STMN2 transcripts that include a cryptic exon.

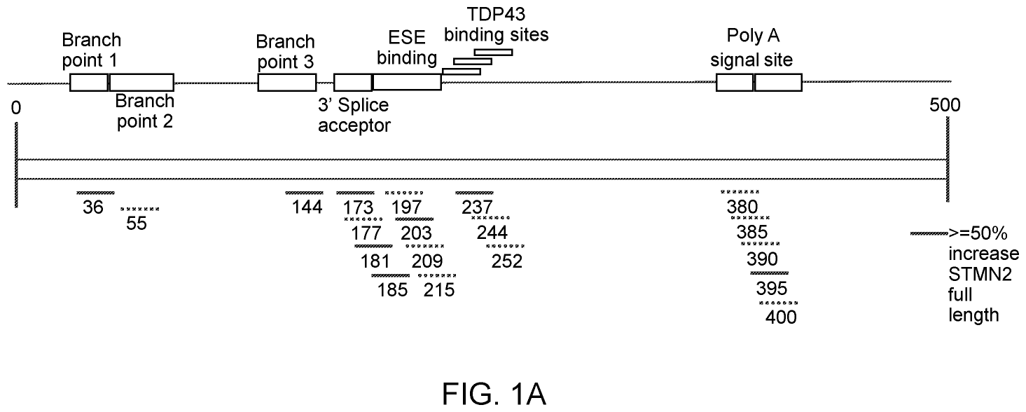

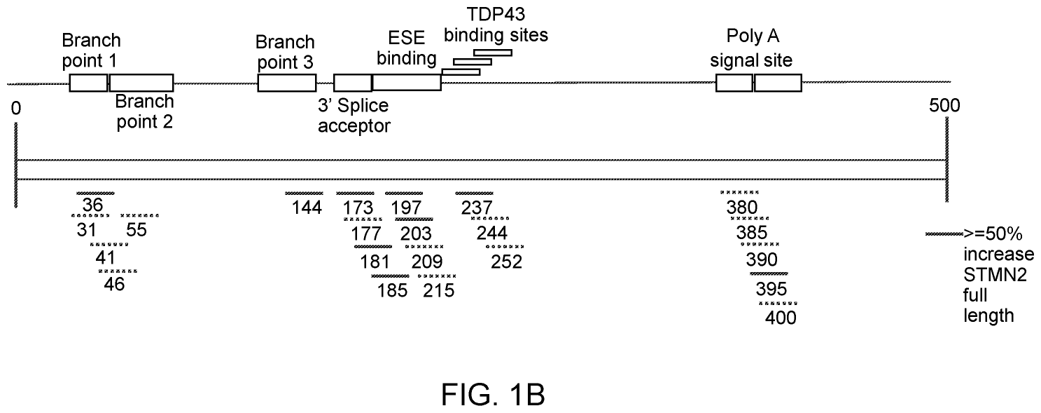

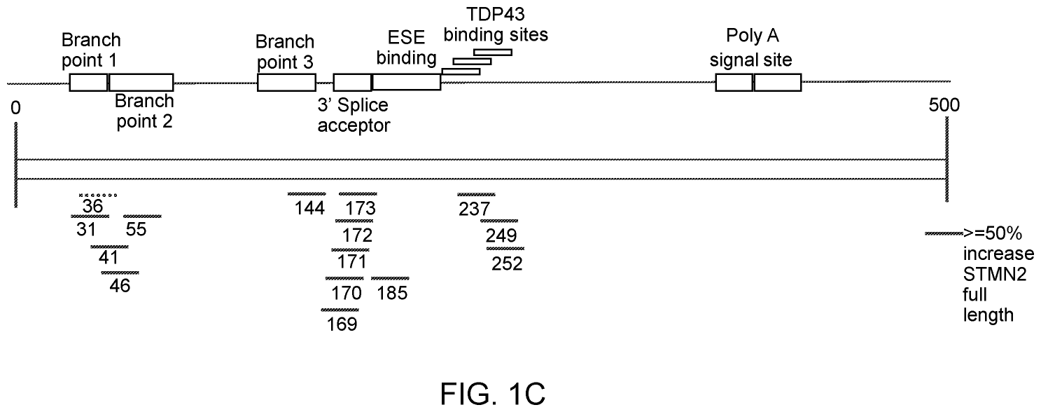

FIGS. 1A-1C depict portions of the STMN2 transcript and STMN2 antisense oligonucleotides that are designed to target certain portions of the STMN2 transcript. Specifically, regions of the STMN2 transcript include branch points (e.g., branch point 1, 2, and 3) a 3′ splice acceptor region, an ESE binding region, TDP43 binding sites, and a Poly A region. STMN2 antisense oligonucleotides, are identified according to the position of the STMN2 transcript that the STMN2 antisense oligonucleotide corresponds to. For example, FIG. 1A depicts a STMN2 antisense oligonucleotide that targets positions 36 to 60 of the STMN2 transcript, which includes a branch point 1. Similarly, a different STMN2 antisense oligonucleotide targets positions 144 to 178 of the STMN2 transcript, which includes a branch point 3. Other STMN2 antisense oligonucleotides can be designed using any of the sequences disclosed above (e.g., SEQ ID NOs: 1-446, 894-918, 945-1390, or 1392-1432).

Generally, the length of the STMN2 antisense oligonucleotides are 25 nucleotides in length. However, variants of the STMN2 antisense oligonucleotides were also designed with varying lengths (e.g., 23 mers, 21 mers, or 19 mers). Specific STMN2 AONs and AON variants that were designed and developed for testing are shown in below in Table 7.

TABLE 7
Identifying information of STMN2 AONs and AON variants including sequence and chemistry information.

| | SEQ ID | | | |
|---|---|---|---|---|
| STMN2 AON | NO: | Sequence (5′-3′) | Internucleoside Linkages | Sugar Moeity |
| QSN-36 | 36 | TTAAAAATGTTAAGACATAATACCA | All phosphorothioate linkages | All nucleosides have 2′MOE sugar |
| | | | | moeity; each “C” is 5-MeC |
| QSN-31 | 31 | AATGTTAAGACATAATACCAGAGCT | All phosphorothioate linkages | All nucleosides have 2′MOE sugar |
| | | | | moeity; each “C” is 5-MeC |
| QSN-41 | 41 | TAGATTTAAAAATGTTAAGACATAA | All phosphorothioate linkages | All nucleosides have 2′MOE sugar |
| | | | | moeity; each “C” is 5-MeC |
| QSN-46 | 46 | TACCATAGATTTAAAAATGTTAAGA | All phosphorothioate linkages | All nucleosides have 2′MOE sugar |
| | | | | moeity; each “C” is 5-MeC |
| QSN-55 | 55 | TGTAAAGATTACCATAGATTTAAAA | All phosphorothioate linkages | All nucleosides have 2′MOE sugar |
| | | | | moeity; each “C” is 5-MeC |
| QSN-144 | 144 | AATCCAATTAAGAGAGAGTGATGGG | All phosphorothioate linkages | All nucleosides have 2′MOE sugar |
| | | | | moeity; each “C” is 5-MeC |
| QSN-146 | 146 | AAAATCCAATTAAGAGAGAGTGATG | All phosphorothioate linkages | All nucleosides have 2′MOE sugar |
| | | | | moeity; each “C” is 5-MeC |
| QSN-150 | 150 | TTTAAAAATCCAATTAAGAGAGAGT | All phosphorothioate linkages | All nucleosides have 2′MOE sugar |
| | | | | moeity; each “C” is 5-MeC |
| QSN-169 | 169 | CCTGCAATATGAATATAATTTTAAA | All phosphorothioate linkages | All nucleosides have 2′MOE sugar |
| | | | | moeity; each “C” is 5-MeC |
| QSN-170 | 170 | TCCTGCAATATGAATATAATTTTAA | All phosphorothioate linkages | All nucleosides have 2′MOE sugar |
| | | | | moeity; each “C” is 5-MeC |
| QSN-171 | 171 | GTCCTGCAATATGAATATAATTTTA | All phosphorothioate linkages | All nucleosides have 2′MOE sugar |
| | | | | moeity; each “C” is 5-MeC |
| QSN-172 | 172 | AGTCCTGCAATATGAATATAATTTT | All phosphorothioate linkages | All nucleosides have 2′MOE sugar |
| | | | | moeity; each “C” is 5-MeC |
| QSN-173 | 173 | GAGTCCTGCAATATGAATATAATTT | All phosphorothioate linkages | All nucleosides have 2′MOE sugar |
| | | | | moeity; each “C” is 5-MeC |
| QSN-177 | 177 | TGCCGAGTCCTGCAATATGAATATA | All phosphorothioate linkages | All nucleosides have 2′MOE sugar |
| | | | | moeity; each “C” is 5-MeC |
| QSN-181 | 181 | CTTCTGCCGAGTCCTGCAATATGAA | All phosphorothioate linkages | All nucleosides have 2′MOE sugar |
| | | | | moeity; each “C” is 5-MeC |
| QSN-185 | 185 | AGGTCTTCTGCCGAGTCCTGCAATA | All phosphorothioate linkages | All nucleosides have 2′MOE sugar |
| | | | | moeity; each “C” is 5-MeC |
| QSN-197 | 197 | CCTTTCTCTCGAAGGTCTTCTGCCG | All phosphorothioate linkages | All nucleosides have 2′MOE sugar |
| | | | | moeity; each “C” is 5-MeC |
| QSN-203 | 203 | TTTCTACCTTTCTCTCGAAGGTCTT | All phosphorothioate linkages | All nucleosides have 2′MOE sugar |
| | | | | moeity; each “C” is 5-MeC |
| QSN-209 | 209 | TCTTATTTTCTACCTTTCTCTCGAA | All phosphorothioate linkages | All nucleosides have 2′MOE sugar |
| | | | | moeity; each “C” is 5-MeC |
| QSN-215 | 215 | CCAAATTCTTATTTTCTACCTTTCT | All phosphorothioate linkages | All nucleosides have 2′MOE sugar |
| | | | | moeity; each “C” is 5-MeC |
| QSN-237 | 237 | GCACACATGCTCACACAGAGAGCCA | All phosphorothioate linkages | All nucleosides have 2′MOE sugar |
| | | | | moeity; each “C” is 5-MeC |
| QSN-244 | 244 | CACACACGCACACATGCTCACACAG | All phosphorothioate linkages | All nucleosides have 2′MOE sugar |
| | | | | moeity; each “C” is 5-MeC |
| QSN-249 | 249 | TCTCGCACACACGCACACATGCTCA | All phosphorothioate linkages | All nucleosides have 2′MOE sugar |
| | | | | moeity; each “C” is 5-MeC |
| QSN-252 | 252 | CTCTCTCGCACACACGCACACATGC | All phosphorothioate linkages | All nucleosides have 2′MOE sugar |
| | | | | moeity; each “C” is 5-MeC |
| QSN-380 | 380 | TGTTTTAATTTCTTCAGTATTGCTA | All phosphorothioate linkages | All nucleosides have 2′MOE sugar |
| | | | | moeity; each “C” is 5-MeC |
| QSN-385 | 385 | TCTTTTGTTTTAATTTCTTCAGTAT | All phosphorothioate linkages | All nucleosides have 2′MOE sugar |
| | | | | moeity; each “C” is 5-MeC |
| QSN-390 | 390 | AGCAATCTTTTGTTTTAATTTCTTC | All phosphorothioate linkages | All nucleosides have 2′MOE sugar |
| | | | | moeity; each “C” is 5-MeC |
| QSN-395 | 395 | GAGACAGCAATCTTTTGTTTTAATT | All phosphorothioate linkages | All nucleosides have 2′MOE sugar |
| | | | | moeity; each “C” is 5-MeC |
| QSN-400 | 400 | ATATTGAGACAGCAATCTTTTGTTT | All phosphorothioate linkages | All nucleosides have 2′MOE sugar |
| | | | | moeity; each “C” is 5-MeC |
| QSN-434 | 434 | CTTAGAATAATTTGGTAAATAATAA | All phosphorothioate linkages | All nucleosides have 2′MOE sugar |
| | | | | moeity; each “C” is 5-MeC |
| QSN-436 | 436 | CTCTTAGAATAATTTGGTAAATAAT | All phosphorothioate linkages | All nucleosides have 2′MOE sugar |
| | | | | moeity; each “C” is 5-MeC |
| QSN-438 | 438 | TACTCTTAGAATAATTTGGTAAATA | All phosphorothioate linkages | All nucleosides have 2′MOE sugar |
| | | | | moeity; each “C” is 5-MeC |
| QSN-440 | 440 | AATACTCTTAGAATAATTTGGTAAA | All phosphorothioate linkages | All nucleosides have 2′MOE sugar |
| | | | | moeity; each “C” is 5-MeC |
| QSN-442 | 442 | GAAATACTCTTAGAATAATTTGGTA | All phosphorothioate linkages | All nucleosides have 2′MOE sugar |
| | | | | moeity; each “C” is 5-MeC |
| QSN-445 | 445 | GAAGAAATACTCTTAGAATAATTTG | All phosphorothioate linkages | All nucleosides have 2′MOE sugar |
| | | | | moeity; each “C” is 5-MeC |
| QSN-446 | 446 | GGAAGAAATACTCTTAGAATAATTT | All phosphorothioate linkages | All nucleosides have 2′MOE sugar |
| | | | | moeity; each “C” is 5-MeC |
| QSN-144-1/5-1/3 | 894 | ATCCAATTAAGAGAGAGTGATGG | All phosphorothioate linkages | All nucleosides have 2′MOE sugar |
| | | | | moeity; each “C” is 5-MeC |
| QSN-144-2/3 | 895 | AATCCAATTAAGAGAGAGTGATG | All phosphorothioate linkages | All nucleosides have 2′MOE sugar |
| | | | | moeity; each “C” is 5-MeC |
| QSN-144-2/5 | 896 | TCCAATTAAGAGAGAGTGATGGG | All phosphorothioate linkages | All nucleosides have 2′MOE sugar |
| | | | | moeity; each “C” is 5-MeC |
| QSN-144-2/5-2/3 | 897 | TCCAATTAAGAGAGAGTGATG | All phosphorothioate linkages | All nucleosides have 2′MOE sugar |
| | | | | moeity; each “C” is 5-MeC |
| QSN-144-3/5-3/3 | 898 | CCAATTAAGAGAGAGTGAT | All phosphorothioate linkages | All nucleosides have 2′MOE sugar |
| | | | | moeity; each “C” is 5-MeC |
| QSN-144-4/3 | 899 | AATCCAATTAAGAGAGAGTGA | All phosphorothioate linkages | All nucleosides have 2′MOE sugar |
| | | | | moeity; each “C” is 5-MeC |
| QSN-144-4/5 | 900 | CAATTAAGAGAGAGTGATGGG | All phosphorothioate linkages | All nucleosides have 2′MOE sugar |
| | | | | moeity; each “C” is 5-MeC |
| QSN-144-p03 | 1419 | A-A-T-CCAATTAAGAGAGAGTGAT-G-G-G | Phosphorothioate linkages except for | All nucleosides have 2′MOE sugar |
| | | | linkages indicated with “-” which are | moeity; each “C” is 5-MeC |
| | | | phosphodiester linkages | |
| QSN-144-p05 | 1420 | AATCCAAT-T-A-A-G-A-GAGAGTGATGGG | Phosphorothioate linkages except for | All nucleosides have 2′MOE sugar |
| | | | linkages indicated with “-” which are | moeity; each “C” is 5-MeC |
| | | | phosphodiester linkages | |
| QSN-173-2/3 | 901 | GAGTCCTGCAATATGAATATAAT | All phosphorothioate linkages | All nucleosides have 2′MOE sugar |
| | | | | moeity; each “C” is 5-MeC |
| QSN-173-2/5 | 902 | GTCCTGCAATATGAATATAATTT | All phosphorothioate linkages | All nucleosides have 2′MOE sugar |
| | | | | moeity; each “C” is 5-MeC |
| QSN-173-2/5-2/3 | 903 | GTCCTGCAATATGAATATAAT | All phosphorothioate linkages | All nucleosides have 2′MOE sugar |
| | | | | moeity; each “C” is 5-MeC |
| QSN-173-4/3 | 904 | GAGTCCTGCAATATGAATATA | All phosphorothioate linkages | All nucleosides have 2′MOE sugar |
| | | | | moeity; each “C” is 5-MeC |
| QSN-173-4/5 | 905 | CCTGCAATATGAATATAATTT | All phosphorothioate linkages | All nucleosides have 2′MOE sugar |
| | | | | moeity; each “C” is 5-MeC |
| QSN-173-6/3 | 906 | GAGTCCTGCAATATGAATA | All phosphorothioate linkages | All nucleosides have 2′MOE sugar |
| | | | | moeity; each “C” is 5-MeC |
| QSN-173-6/5 | 907 | TGCAATATGAATATAATTT | All phosphorothioate linkages | All nucleosides have 2′MOE sugar |
| | | | | moeity; each “C” is 5-MeC |
| QSN-173-po3 | 1417 | G-A-G-TCCTGCAATATGAATATAA-T-T-T | Phosphorothioate linkages except for | All nucleosides have 2′MOE sugar |
| | | | linkages indicated with “-” which are | moeity; each “C” is 5-MeC |
| | | | phosphodiester linkages | |
| QSN-173-po5 | 1418 | GAGTCCTG-C-A-A-T-A-TGAATATAATTT | Phosphorothioate linkages except for | All nucleosides have 2′MOE sugar |
| | | | linkages indicated with “-” which are | moeity; each “C” is 5-MeC |
| | | | phosphodiester linkages | |
| QSN-185-2/5 | 908 | GTCTTCTGCCGAGTCCTGCAATA | All phosphorothioate linkages | All nucleosides have 2′MOE sugar |
| | | | | moeity; each “C” is 5-MeC |
| QSN-185-4/3 | 909 | AGGTCTTCTGCCGAGTCCTGC | All phosphorothioate linkages | All nucleosides have 2′MOE sugar |
| | | | | moeity; each “C” is 5-MeC |
| QSN-185-4/5 | 910 | CTTCTGCCGAGTCCTGCAATA | All phosphorothioate linkages | All nucleosides have 2′MOE sugar |
| | | | | moeity; each “C” is 5-MeC |
| QSN-185-6/5 | 911 | TCTGCCGAGTCCTGCAATA | All phosphorothioate linkages | All nucleosides have 2′MOE sugar |
| | | | | moeity; each “C” is 5-MeC |
| QSN-185-po3 | 1421 | A-G-G-TCTTCTGCCGAGTCCTGCA-A-T-A | Phosphorothioate linkages except for | All nucleosides have 2′MOE sugar |
| | | | linkages indicated with “-” which are | moeity; each “C” is 5-MeC |
| | | | phosphodiester linkages | |
| QSN-185-po5 | 1422 | AGGTCTTC-T-G-C-C-G-AGTCCTGCAATA | Phosphorothioate linkages except for | All nucleosides have 2′MOE sugar |
| | | | linkages indicated with “-” which are | moeity; each “C” is 5-MeC |
| | | | phosphodiester linkages | |
| QSN-237-2-3p | 912 | GCACACATGCTCACACAGAGAGC | All phosphorothioate linkages | All nucleosides have 2′MOE sugar |
| | | | | moeity; each “C” is 5-MeC |
| QSN-237-2-5p | 912 | ACACATGCTCACACAGAGAGCCA | All phosphorothioate linkages | All nucleosides have 2′MOE sugar |
| | | | | moeity; each “C” is 5-MeC |
| QSN-237-2-5p- | 914 | ACACATGCTCACACAGAGAGC | All phosphorothioate linkages | All nucleosides have 2′MOE sugar |
| 2-3p | | | | moeity; each “C” is 5-MeC |
| QSN-237-4-3p | 915 | GCACACATGCTCACACAGAGA | All phosphorothioate linkages | All nucleosides have 2′MOE sugar |
| | | | | moeity; each “C” is 5-MeC |
| QSN-237-4-5p | 916 | ACATGCTCACACAGAGAGCCA | All phosphorothioate linkages | All nucleosides have 2′MOE sugar |
| | | | | moeity; each “C” is 5-MeC |
| QSN-237-6-3p | 917 | GCACACATGCTCACACAGA | All phosphorothioate linkages | All nucleosides have 2′MOE sugar |
| | | | | moeity; each “C” is 5-MeC |
| QSN-237-6-5p | 918 | ATGCTCACACAGAGAGCCA | All phosphorothioate linkages | All nucleosides have 2′MOE sugar |
| | | | | moeity; each “C” is 5-MeC |
| QSN-237-po3 | 1423 | G-C-A-CACATGCTCACACAGAGAG-C-C-A | Phosphorothioate linkages except for | All nucleosides have 2′MOE sugar |
| | | | linkages indicated with “-” which are | moeity; each “C” is 5-MeC |
| | | | phosphodiester linkages | |
| QSN-237-p05 | 1424 | GCACACAT-G-C-T-C-A-CACAGAGAGCCA | Phosphorothioate linkages except for | All nucleosides have 2′MOE sugar |
| | | | linkages indicated with “-” which are | moeity; each “C” is 5-MeC |
| | | | phosphodiester linkages | |

### Example 2: Methods for Evaluating STMN2 Antisense Oligonucleotides

STMN2 antisense oligonucleotides were evaluated in SY5Y cells and human motor neurons. Specifically, Examples 3, 4, and 5 below describe results generated from evaluation of STMN2 antisense oligonucleotides in SY5Y cells. Example 6 and 7 below describe results generated from evaluation of STMN2 antisense oligonucleotides in human motor neurons.

STMN2 antisense oligonucleotides were evaluated in SY5Y cells. The cells were plated in 6-well or 96-well plates and cultured to 80% confluency. Antisense oligonucleotide (AON) to TDP43 was transfected with RNAiMax (Thermo Fisher Scientific, Waltham, Mass., USA) to express the cryptic exon, thus preventing transcription of full-length STMN2 (STMN2-FL) product. Vehicle was treated with RNAiMax alone. Positive controls included cells that were treated with TDP43 siRNA alone (“siRNA TDP43”) and/or TDP43 AON alone (“AON TDP43” or “TDP43 AON”). siRNA TDP43 was purchased as ON-TARGETplus Human TARDBP (23435) siRNA-SMARTpool (#L-012394-00-0005) from Horizon/Dharmacon. TARDBP (23435) siRNA includes four individual siRNAs that targets four separate sequences:

(SEQ ID NO: 1439)
1) Target sequence 1: GCUCAAGCAUGGAUUCUAA
(SEQ ID NO: 1440)
2) Target sequence 2: CAAUCAAGGUAGUAAUAUG
(SEQ ID NO: 1441)
3) Target sequence 3: GGGCUUCGCUACAGGAAUC
(SEQ ID NO: 1442)
4) Target sequence 4: CAGGGUGGAUUUGGUAAUA

TDP43 AON is a gapmer oligonucleotide and has the following sequence and chemistry:

(SEQ ID NO: 1443)
5′ A*A*G*G*C*T*T*C*A*T*A*T*T*G*T*A*C*T*T*T 3′

where *=phosphorothioate, underlined=DNA, other=2′MOE RNA; each “C” is 5-MeC.

To evaluate STMN2 AON ability to restore STMN2-FL, antisense oligonucleotides to STMN2 were co-incubated with TDP43 AON in RNAiMax. After 96 hours, transcript levels (e.g., STMN2 full length transcript, STMN2 transcript with cryptic exon, or TDP43 transcript) were detected by RT-qPCR using Taqman. Specifically, RT-qPCR was performed for detecting GAPDH using Thermofisher TaqMan Gene Expression Assay Hs03929097_g1. RT-qPCR was performed for detecting STMN2 transcripts with cryptic exon using the following primer sequences: 1) Forward primer: 5′-CTCAGTGCCTTATTCAGTCTTCTC-3′ (SEQ ID NO: 1444), 2) Reverse primer: 5′-TCTTCTGCCGAGTCCCATTT-3′ (SEQ ID NO: 1445) and 3) Probe: 5′-/56-FAM/TCAGCGTCTGCACATCCCTACAAT/3BHQ_1/-3′ (SEQ ID NO: 1446). RT-qPCR was performed for detecting full length STMN2 transcripts using the following primer sequences: 1) Forward primer: 5′-CCACGAACTTTAGCTTCTCCA-3′ (SEQ ID NO: 1447), 2) Reverse primer: 5′-GCCAATTGTTTCAGCACCTG-3′ (SEQ ID NO: 1448), and 3) Probe: 5′-/56-FAM/ACTTTCTTCTTTCCTCTGCAGCCTCC/3BHQ_1/-3′ (SEQ ID NO: 1449).

RT-qPCR was performed on Applied Biosystems® 7500 Real-time PCR systems. One cycle of reverse transcription was performed at a temperature of 50° C. for 5 min. One cycle of RT inactivation/initial denaturation was performed at a temperature of 95° C. for 20 seconds. Forty five cycles of amplification were performed at a temperature of 95° C. for 1 second followed by 60° C. for 20 seconds.

STMN2-FL or STMN2 cryptic signal (Ct) was normalized to GAPDH (deltaCt). To visualize the quantitative changes (e.g., % increase of STMN-FL), the normalized STMN2-FL signal was further normalized to the vehicle (treated with RNAiMax alone, deltadeltaCt). Relative quantity of transcript level was calculated using the equation RQ=2^{-deltadeltaCt} and is used to describe the treatment condition comparison to normal, healthy levels (1.0).

Percent decrease of STMN2 with cryptic exon expression was calculated using the equation of:

$$100 - ( \frac{Mean relative quantity of STMN 2 with \\ cryptic exon in response to STMN 2 A O N}{Mean relative quantity of S TMN 2 with \\ cryptic exon in response to TD P 43 A O N} ⋆ 100 )$$

The percent increase of full length STMN2 mRNA transcript was calculated using the equation of:

$$( \frac{Mean relative quantity of FL S TMN 2 transcript \\ in response to S TMN 2 A O N}{Mean relative quantity of FL S TMN 2 transcript \\ in response to TD P 43 A O N} ) ⋆ 100$$

STMN2 antisense oligonucleotides were also evaluated in human motor neurons for potency in reducing cryptic exon and increasing STMN2 full length transcript. iCell human motor neurons (Cellular Dynamics International) were plated at 15×10³ cells/well in a 96-well plate for RT-qPCR. RNA quantification or 3×10⁵ cells/well in a 6-well plate for western blot protein quantification according to manufacturer's instructions. Neurons were transfected with TDP43 AON and/or STMN2 AON using endoporter (GeneTools, LLC.) or treated with endoporter alone. Treatment conditions were tested in biological triplicate (qRT-PCR) or duplicate (western blot) wells. The same TDP43 AON described above is used here for evaluating human motor neurons. TDP43 AON is a gapmer oligonucleotide and has the following sequence and chemistry:

(SEQ ID NO: 1443)
5′ A*A*G*G*C*T*T*C*A*T*A*T*T*G*T*A*C*T*T*T 3′

where*phosphorothioate, underlined DNA, other=2′MOE RNA; each “C” is 5-MeC.

After 72 hours, antisense oligonucleotides and endoporter were washed out and replaced with fresh media. After 72 additional hours, RNA was collected from the 96-well plates for RT-qPCR or protein collected from the 6-well plates for western blot. RNA was isolated, cDNA generated and multiplexed RT-qPCR assay performed with taqman probes for STMN2 cryptic exon, STMNN2 full length transcript and reference GAPDH quantification. The same primers for detecting GAPDH, STMN2 transcript with cryptic exon, and fill length STMN2, as described above in reference to SYSY cells, were applied here for conducting RT-qPCR for human motor neurons. For protein quantification, the soluble portion of the protein collection was denatured and separated by SDS-PAGE, transferred to polyvinylidene difluoride membranes and probed with antibodies against GAPDH (Proteintech, 60004-1-1g), TDP-43 (Proteintech, 10782-2-AP), and Stathmin-2 (ThermoFisher, PAS-23049).

STMN2 antisense oligonucleotides were tested for their ability to increase or restore full-length STMN2 mRNA (i.e., mRNA from which full-length STMN2 is translated) levels in TDP43 silenced cells (e.g., SYSY cells and human motor neurons). In some cases, STMN2 antisense oligonucleotides were tested for their ability to reduce STMN2 transcripts with cryptic exon. As described further below, the quantified percentage increase/restoration of STMN2-FL and/or percentage reduction of STMN2 transcripts with cryptic exon is described in reference to levels of STMN-FL and/or STMN2 transcripts with cryptic exon in a control group (e.g., cells treated with 500 nM TDP43 AON).

### Example 3: STMN2 Antisense Oligonucleotides Restore Full Length STMN2 and Reduce STMN2 Transcripts with Cryptic Exon in SY5Y cells

FIGS. 1B and 1C demonstrate the effectiveness of STMN2 AONs targeting different regions of the STMN2 transcript with cryptic exon. In particular, FIG. 1B depicts STMN2 AONs that were designed and evaluated in SYSY cells. FIG. 1C depicts STMN2 AONs that were designed and evaluated in human motor neurons. STMN2 AONs represented by a solid line resulted in cells with increased STMN2-FL mRNA expression by greater than 50% over TDP43 AON treated alone. STMN2 AONs represented by a dotted line resulted in cells with increased STMN2-FL (full length) mRNA by less than 50% over TDP43 AON treated alone.

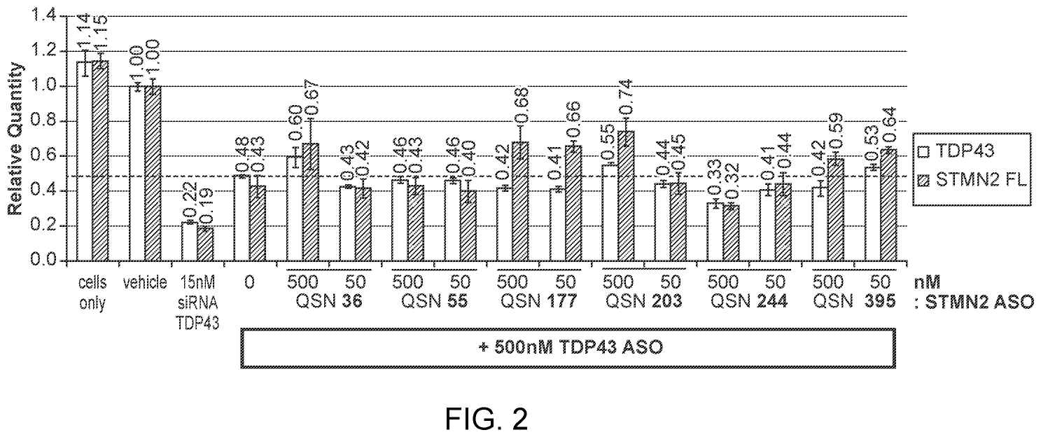

Referring to FIG. 2, TDP43 transcript was decreased by around 52% and STMN2-FL was decreased by around 57% when treated with 500 nM TDP43 AON. A 500 nM treatment of a STMN2 AON with SEQ ID NO: 36 increased TDP43 levels by 25% and increased STMN-FL levels by 55% (rescued to 67%). A 50 nM and a 500 nM treatment of a STMN2 AON with SEQ ID NO: 177 increased STMN-FL levels by 58% (rescued to 68%) and 53% (rescued to 66%) respectively. A 500 nM treatment of a STMN2 AON with SEQ ID NO: 203 increased TDP43 levels by 15% and STMN-FL levels by 72% (rescued to 74%). A 50 nM and a 500 nM treatment of a STMN2 AON with SEQ ID NO: 395 increased STMN-FL levels by 49% (rescued to 64%) and 37% (rescued to 59%) respectively. Dotted line represents level of expression of FL-STMN2 in response to 500 nM TDP43 AON.

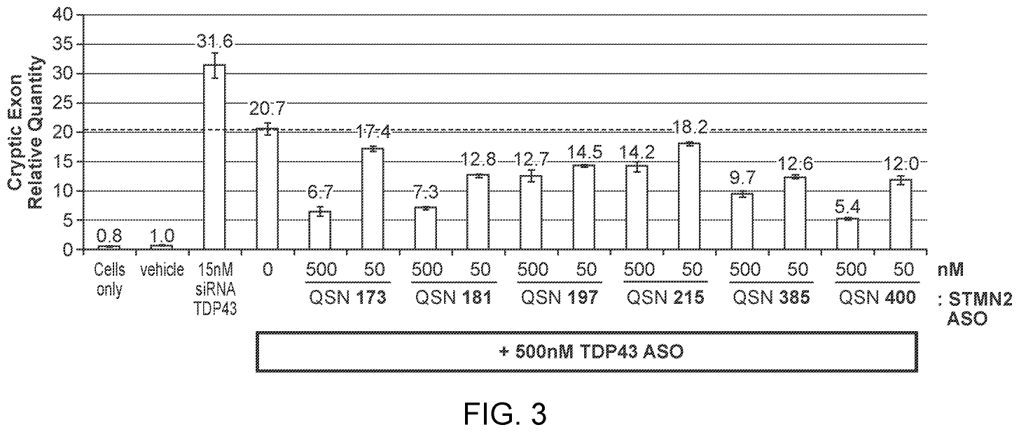

Referring to FIG. 3, the quantity of STMN2 transcript with cryptic exon was increased more than 20-fold when treated with 500 nM TDP43 AON. A 500 nM treatment of a STMN2 AON with SEQ ID NO: 173 reduced STMN2 transcript with cryptic exon levels by 68%. A 500 nM treatment of a STMN2 AON with SEQ ID NO: 181 reduced STMN2 transcript with cryptic exon levels by 65%. A 500 nM treatment of a STMN2 AON with SEQ ID NO: 197 reduced STMN2 transcript with cryptic exon levels by 39%. A 500 nM treatment of a STMN2 AON with SEQ ID NO: 215 reduced STMN2 transcript with cryptic exon levels by 31%. A 500 nM treatment of a STMN2 AON with SEQ ID NO: 385 reduced STMN2 transcript with cryptic exon levels by 53%. A 500 nM treatment of a STMN2 AON with SEQ ID NO: 400 reduced STMN2 transcript with cryptic exon levels by 74%. Dotted line represents level of expression of STMN2 with cryptic exon in response to 500 nM TDP43 AON.

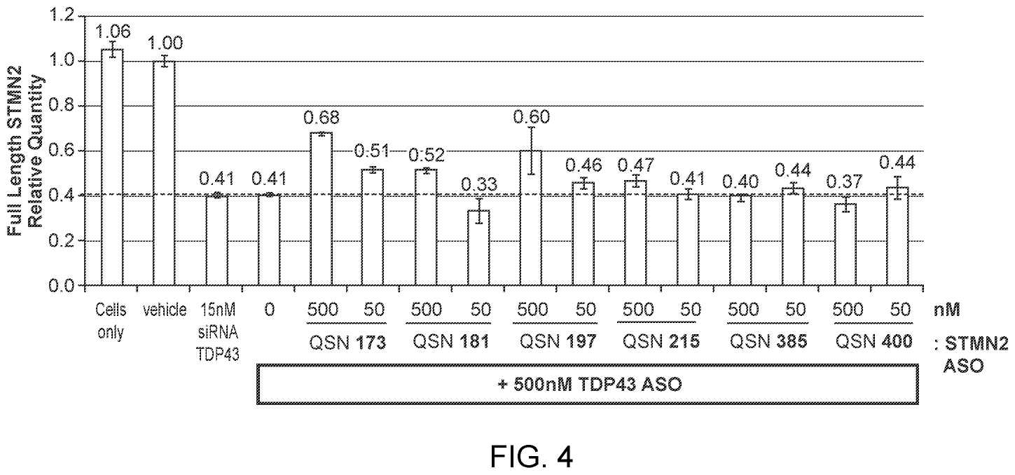

Referring to FIG. 4, STMN2-FL was decreased by around 59% when treated with 500 nM TDP43 AON. A 500 nM treatment of a STMN2 AON with SEQ ID NO: 173 increased STMN-FL levels to 166% (rescued to 68%). A 500 nM treatment of a STMN2 AON with SEQ ID NO: 197 increased STMN-FL levels to 146% (rescued to 60%). Dotted line represents level of expression of FL-STMN2 in response to 500 nM TDP43 AON.

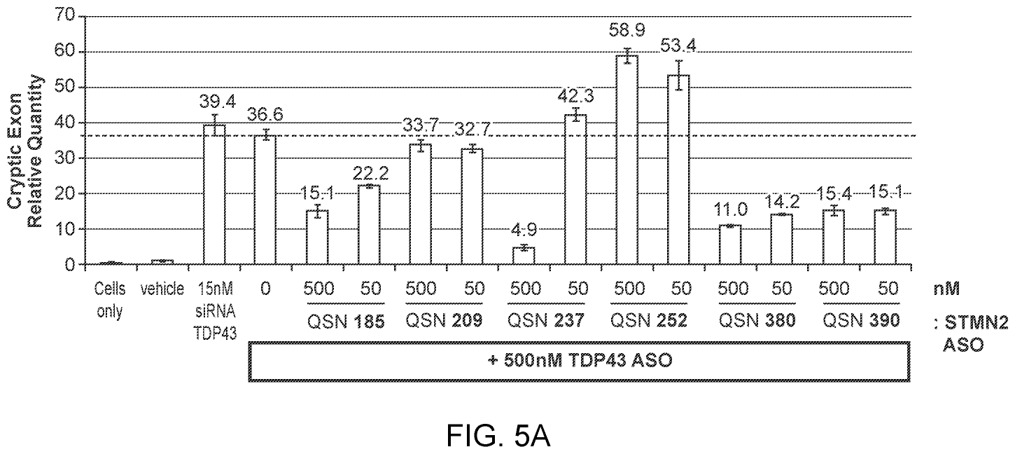

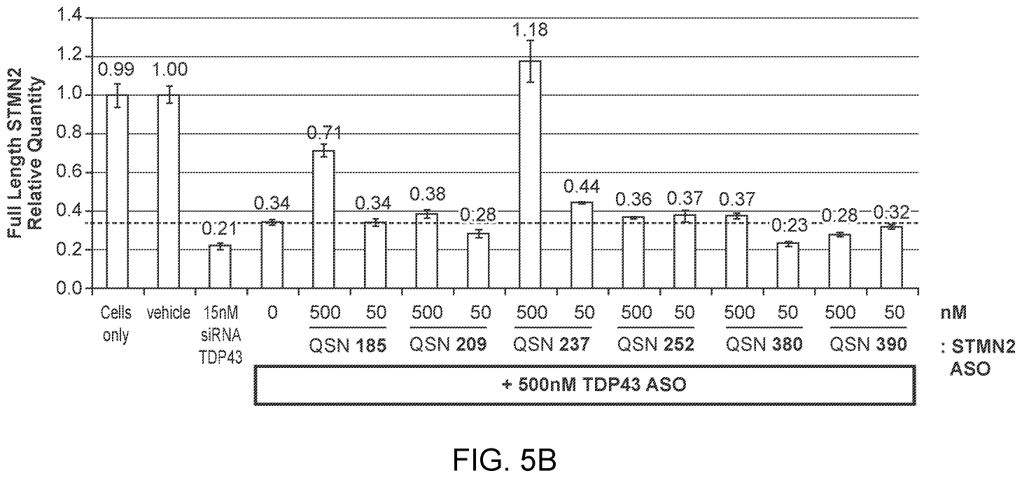

Referring to FIG. 5A, the quantity of STMN2 transcript with cryptic exon was increased more than 36-fold when treated with 500 nM TDP43 AON. A 500 nM treatment of a STMN2 AON with SEQ ID NO: 185 reduced STMN2 transcript with cryptic exon levels by 58%. A 500 nM treatment of a STMN2 AON with SEQ ID NO: 237 reduced STMN2 transcript with cryptic exon levels by 87%. A 500 nM treatment of a STMN2 AON with SEQ ID NO: 380 reduced STMN2 transcript with cryptic exon levels by 70%. A 500 nM treatment of a STMN2 AON with SEQ ID NO: 390 reduced STMN2 transcript with cryptic exon levels by 58%. Dotted line represents level of expression of STMN2 with cryptic exon in response to 500 nM TDP43 AON.

Referring to FIG. 5B, STMN2-FL was decreased by 66% when treated with 500 nM TDP43 AON. A 500 nM treatment of a STMN2 AON with SEQ ID NO: 185 increased STMN-FL levels to 209% (rescued to 71%). A 500 nM treatment of a STMN2 AON with SEQ ID NO: 237 increased STMN-FL levels to 347% (rescued to 118%). Dotted line represents level of expression of FL-STMN2 in response to 500 nM TDP43 AON.

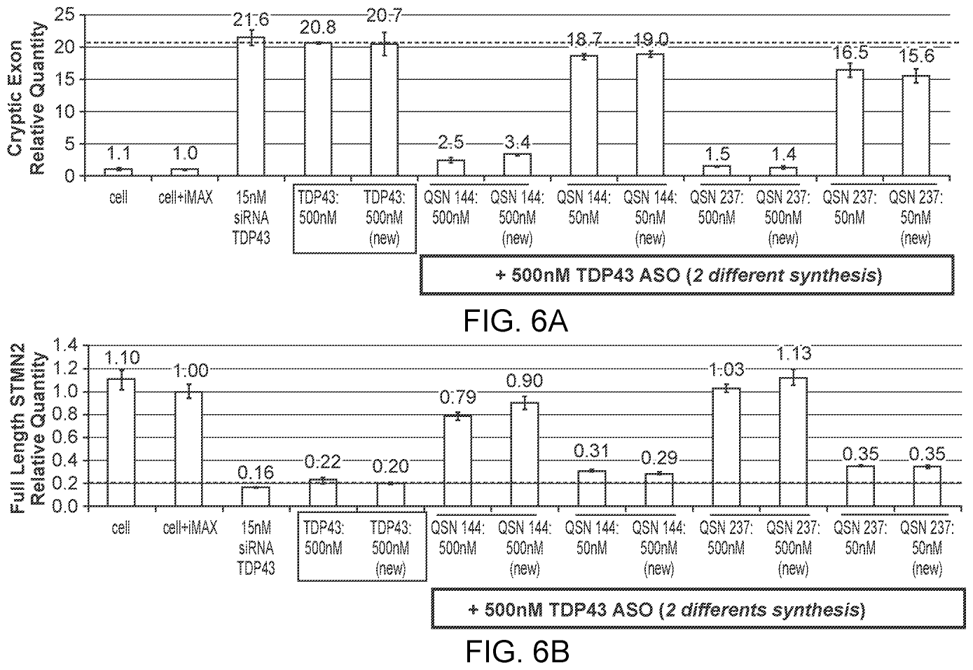

Referring to FIG. 6A, the quantity of STMN2 transcript with cryptic exon was increased more than 20-fold when treated with 500 nM TDP43 AON (two different syntheses). A 500 nM treatment of a STMN2 AON with SEQ ID NO: 144 reduced STMN2 transcript with cryptic exon levels by 83 to 88%. A 500 nM treatment of a STMN2 AON with SEQ ID NO: 237 reduced STMN2 transcript with cryptic exon levels by 92 to 93%. Dotted line represents level of expression of STMN2 with cryptic exon in response to 500 nM TDP43 AON.

Referring to FIG. 6B, STMN2-FL was decreased by about 80% when treated with 500 nM TDP43 AON. A 500 nM treatment of a STMN2 AON with SEQ ID NO: 144 increased STMN-FL levels to between 376% and 429% (rescued to between 79% to 90%). A 500 nM treatment of a STMN2 AON with SEQ ID NO: 237 increased STMN-FL levels to between 490% and 538% (rescued to 103% to 113%). Dotted line represents level of expression of FL-STMN2 in response to 500 nM TDP43 AON.

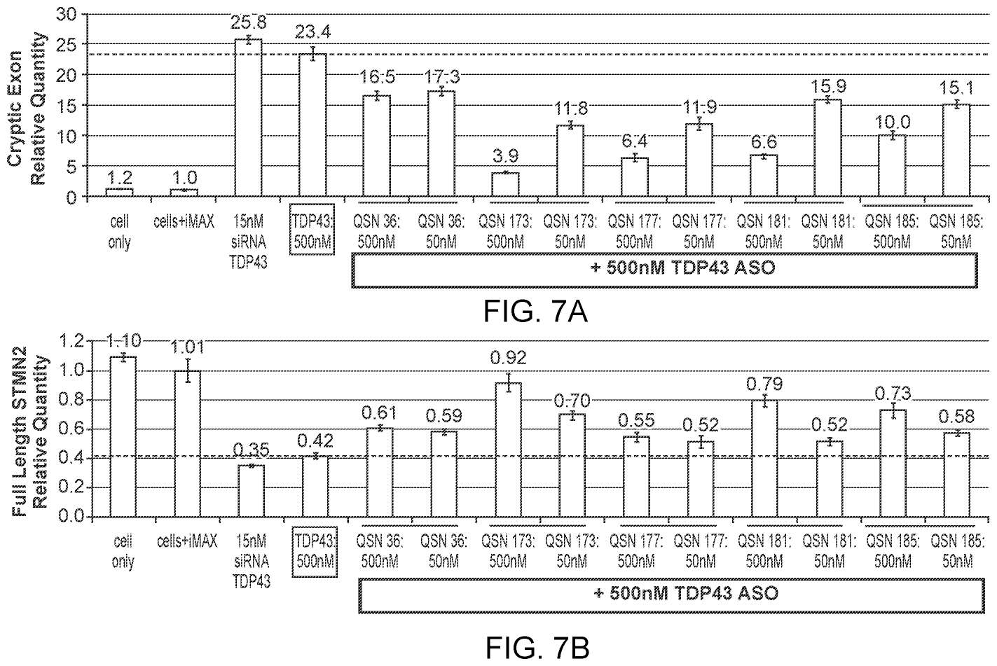

Referring to FIG. 7A, the quantity of STMN2 transcript with cryptic exon was increased more than 23-fold when treated with 500 nM TDP43 AON. A 500 nM treatment of a STMN2 AON with SEQ ID NO: 173 reduced STMN2 transcript with cryptic exon levels by 83%. A 500 nM treatment of a STMN2 AON with SEQ ID NO: 177 reduced STMN2 transcript with cryptic exon levels by 83%. A 500 nM treatment of a STMN2 AON with SEQ ID NO: 181 reduced STMN2 transcript with cryptic exon levels by 72%. Dotted line represents level of expression of STMN2 with cryptic exon in response to 500 nM TDP43 AON.

Referring to FIG. 7B, STMN2-FL was decreased by about 58% when treated with 500 nM TDP43 AON. A 500 nM treatment of a STMN2 AON with SEQ ID NO: 173 increased STMN-FL levels to 219% (rescued to 92%). A 500 nM treatment of a STMN2 AON with SEQ ID NO: 181 increased STMN-FL levels to 188% (rescued to 79%). A 500 nM treatment of a STMN2 AON with SEQ ID NO: 185 increased STMN-FL levels to 174% (rescued to 73%). Dotted line represents level of expression of FL-STMN2 in response to 500 nM TDP43 AON.

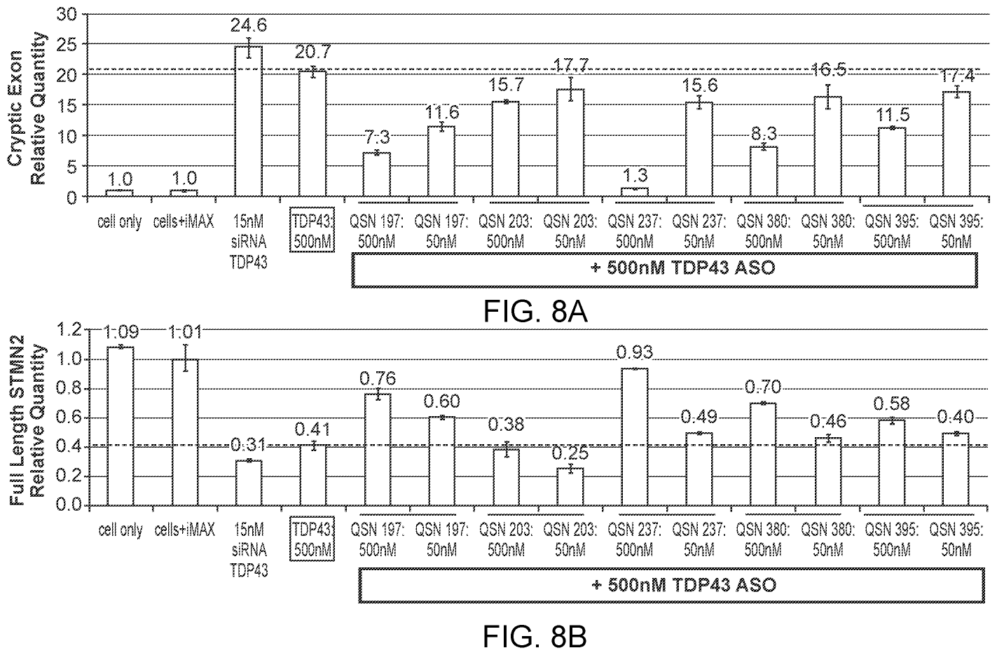

Referring to FIG. 8A, the quantity of STMN2 transcript with cryptic exon was increased more than 20-fold when treated with 500 nM TDP43 AON. A 500 nM treatment of a STMN2 AON with SEQ ID NO: 197 reduced STMN2 transcript with cryptic exon levels by 65%. A 500 nM treatment of a STMN2 AON with SEQ ID NO: 237 reduced STMN2 transcript with cryptic exon levels by 94%. Dotted line represents level of expression of STMN2 with cryptic exon in response to 500 nM TDP43 AON.

Referring to FIG. 8B, STMN2-FL was decreased by 59% when treated with 500 nM TDP43 AON. A 500 nM treatment of a STMN2 AON with SEQ ID NO: 197 increased STMN-FL levels to 185% (rescued to 76%). A 500 nM treatment of a STMN2 AON with SEQ ID NO: 237 increased STMN-FL levels to 227% (rescued to 93%). A 500 nM treatment of a STMN2 AON with SEQ ID NO: 380 increased STMN-FL levels to 171% (rescued to 70%). Dotted line represents level of expression of FL-STMN2 in response to 500 nM TDP43 AON.

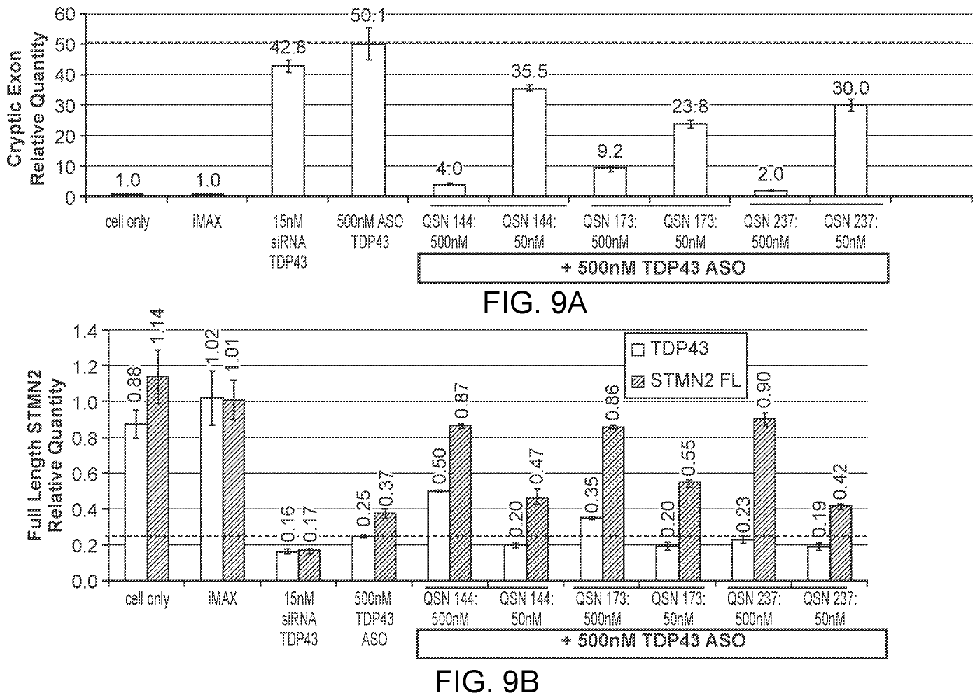

Referring to FIG. 9A, the quantity of STMN2 transcript with cryptic exon was increased more than 50-fold when treated with 500 nM TDP43 AON. A 500 nM treatment of a STMN2 AON with SEQ ID NO: 144 reduced STMN2 transcript with cryptic exon levels by 92%. A 500 nM treatment of a STMN2 AON with SEQ ID NO: 173 reduced STMN2 transcript with cryptic exon levels by 82%. A 500 nM treatment of a STMN2 AON with SEQ ID NO: 237 reduced STMN2 transcript with cryptic exon levels by 96%. Dotted line represents level of expression of STMN2 with cryptic exon in response to 500 nM TDP43 AON.

Referring to FIG. 9B, STMN2-FL was decreased by 67% when treated with 500 nM TDP43 AON. A 500 nM treatment of a STMN2 AON with SEQ ID NO: 144 increased STMN-FL levels to 235% (rescued to 87%). A 500 nM treatment of a STMN2 AON with SEQ ID NO: 173 increased STMN-FL levels to 232% (rescued to 86%). A 500 nM treatment of a STMN2 AON with SEQ ID NO: 237 increased STMN-FL levels to 243% (rescued to 90%). Dotted line represents level of expression of FL-STMN2 in response to 500 nM TDP43 AON.

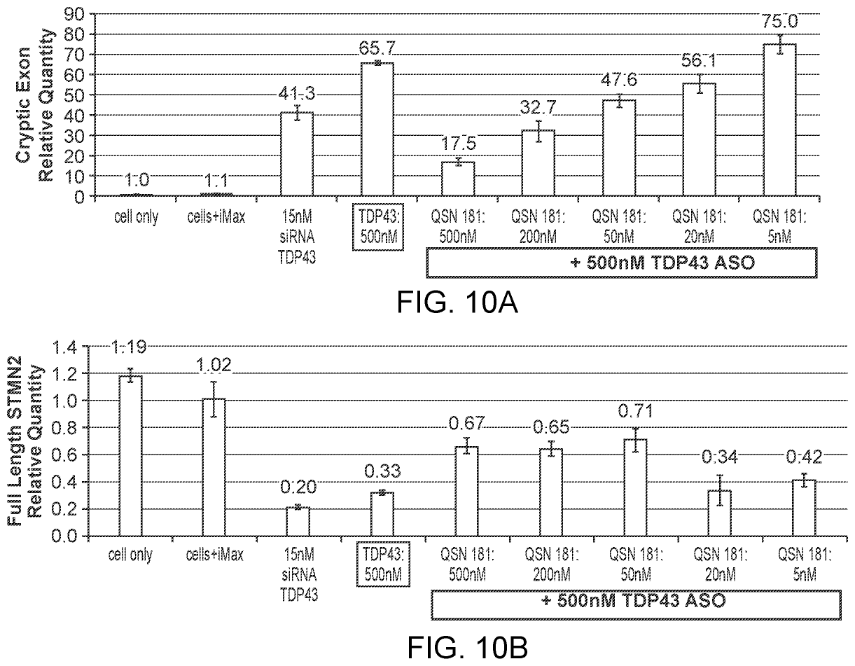

Referring to FIG. 10A, the quantity of STMN2 transcript with cryptic exon was increased more than 65-fold when treated with 500 nM TDP43 AON. A 200 nM treatment of a STMN2 AON with SEQ ID NO: 181 reduced STMN2 transcript with cryptic exon levels by 50%. A 500 nM treatment of a STMN2 AON with SEQ ID NO: 181 reduced STMN2 transcript with cryptic exon levels by 73%. Referring to FIG. 10B, STMN2-FL was decreased by 67% when treated with 500 nM TDP43 AON. A 50 nM treatment of a STMN2 AON with SEQ ID NO: 181 increased STMN-FL levels to 215% (rescued to 71%). A 200 nM treatment of a STMN2 AON with SEQ ID NO: 181 increased STMN-FL levels to 197% (rescued to 65%). A 500 nM treatment of a STMN2 AON with SEQ ID NO: 181 increased STMN-FL levels to 194% (rescued to 64%).

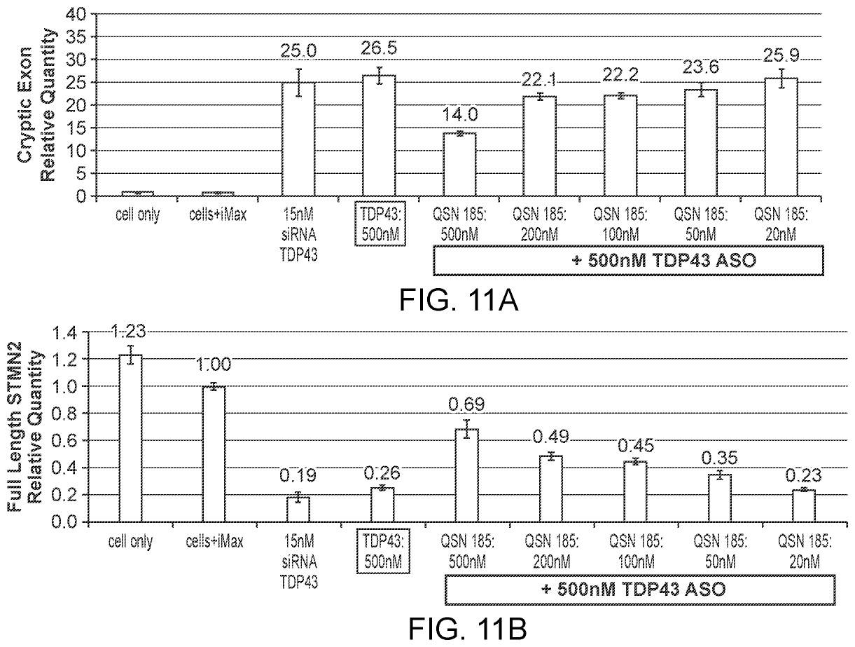

Referring to FIG. 11A, the quantity of STMN2 transcript with cryptic exon was increased more than 26-fold when treated with 500 nM TDP43 AON. A 500 nM treatment of a STMN2 AON with SEQ ID NO: 185 reduced STMN2 transcript with cryptic exon levels by 47%. Referring to FIG. 11B, STMN2-FL was decreased by 74% when treated with 500 nM TDP43 AON. A 50 nM treatment of a STMN2 AON with SEQ ID NO: 185 increased STMN-FL levels to 173% (rescued to 45%). A 200 nM treatment of a STMN2 AON with SEQ ID NO: 185 increased STMN-FL levels to 346% (rescued to 90%). A 500 nM treatment of a STMN2 AON with SEQ ID NO: 185 increased STMN-FL levels to 265% (rescued to 69%).

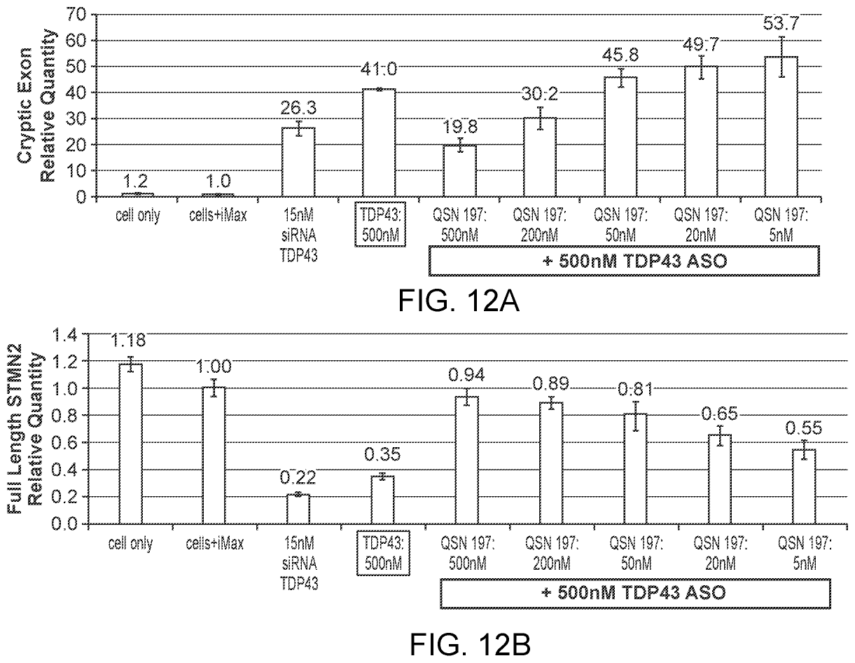

Referring to FIG. 12A, the quantity of STMN2 transcript with cryptic exon was increased more than 41-fold when treated with 500 nM TDP43 AON. A 500 nM treatment of a STMN2 AON with SEQ ID NO: 197 reduced STMN2 transcript with cryptic exon levels by 51%. Referring to FIG. 12B, STMN2-FL was decreased by 65% when treated with 500 nM TDP43 AON. A 20 nM treatment of a STMN2 AON with SEQ ID NO: 197 increased STMN-FL levels to 186% (rescued to 65%). A 50 nM treatment of a STMN2 AON with SEQ ID NO: 197 increased STMN-FL levels to 231% (rescued to 81%). A 200 nM treatment of a STMN2 AON with SEQ ID NO: 197 increased STMN-FL levels to 254% (rescued to 89%). A 500 nM treatment of a STMN2 AON with SEQ ID NO: 197 increased STMN-FL levels to 269% (rescued to 94%).

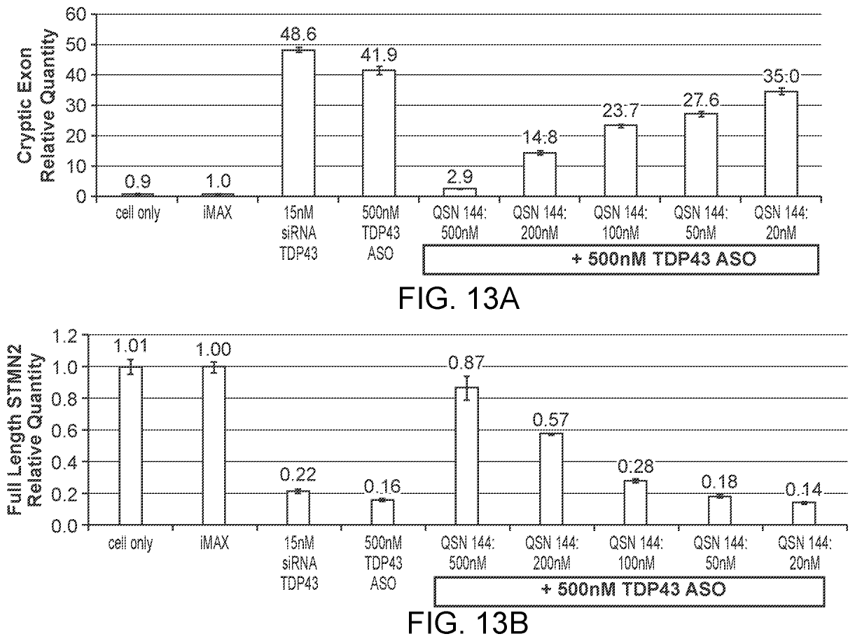

Referring to FIG. 13A, the quantity of STMN2 transcript with cryptic exon was increased more than 41-fold when treated with 500 nM TDP43 AON. A 500 nM treatment of a STMN2 AON with SEQ ID NO: 144 reduced STMN2 transcript with cryptic exon levels by 93%. Referring to FIG. 13B, STMN2-FL was decreased by 84% when treated with 500 nM TDP43 AON. A 50 nM treatment of a STMN2 AON with SEQ ID NO: 144 increased STMN-FL levels to 175% (rescued to 28%). A 200 nM treatment of a STMN2 AON with SEQ ID NO: 144 increased STMN-FL levels to 360% (rescued to 57%). A 500 nM treatment of a STMN2 AON with SEQ ID NO: 144 increased STMN-FL levels to 544% (rescued to 87%).

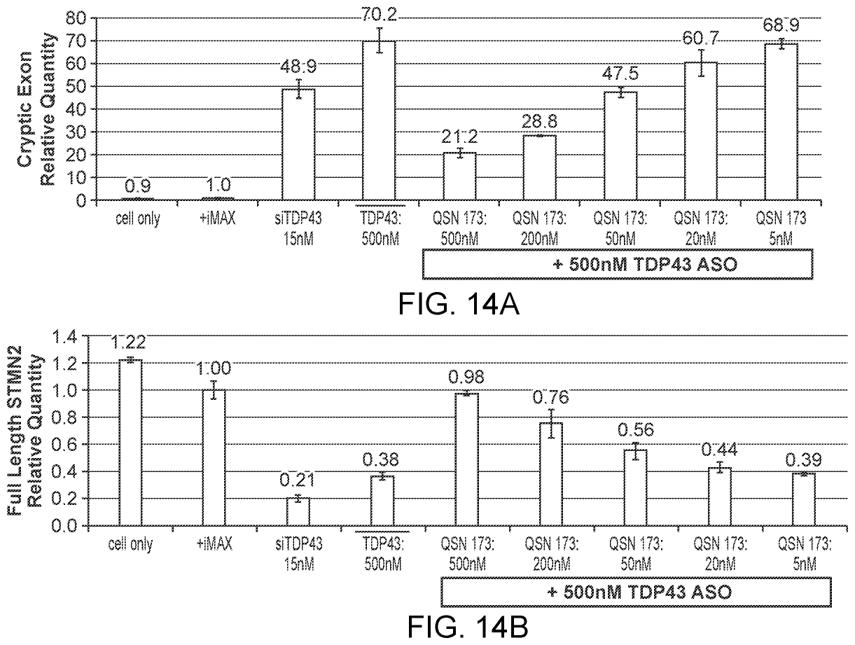

Referring to FIG. 14A, the quantity of STMN2 transcript with cryptic exon was increased more than 70-fold when treated with 500 nM TDP43 AON. A 200 nM treatment of a STMN2 AON with SEQ ID NO: 173 reduced STMN2 transcript with cryptic exon levels by 59%. A 500 nM treatment of a STMN2 AON with SEQ ID NO: 173 reduced STMN2 transcript with cryptic exon levels by 70%. Referring to FIG. 14B, STMN2-FL was decreased by 62% when treated with 500 nM TDP43 AON. A 200 nM treatment of a STMN2 AON with SEQ ID NO: 173 increased STMN-FL levels by 100% (rescued to 76%). A 500 nM treatment of a STMN2 AON with SEQ ID NO: 173 increased STMN-FL levels by 158% (rescued to 98%).

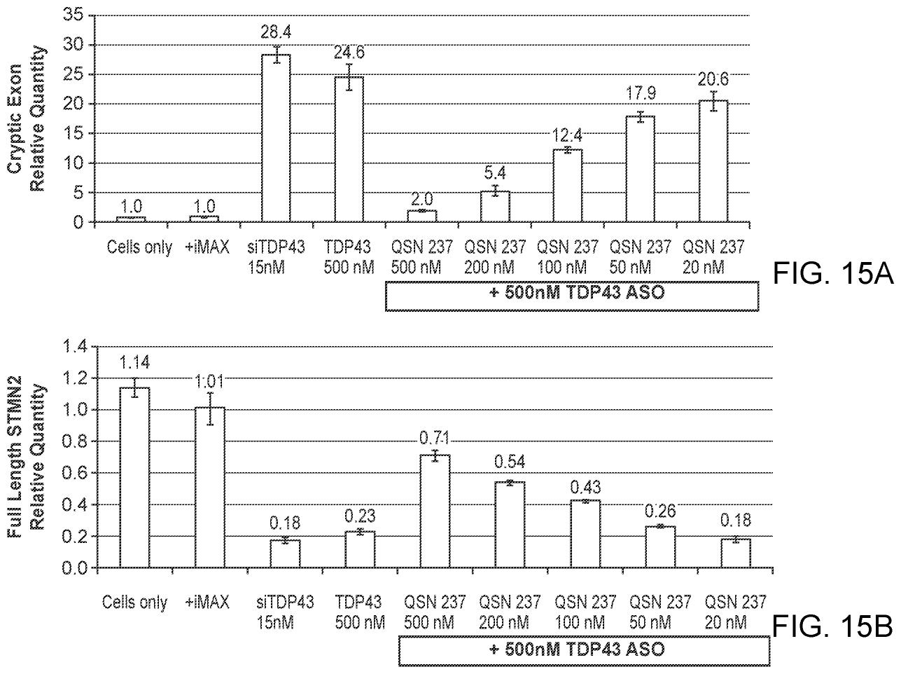

Referring to FIG. 15A, the quantity of STMN2 transcript with cryptic exon was increased more than 70-fold when treated with 500 nM TDP43 AON. A 200 nM treatment of a STMN2 AON with SEQ ID NO: 237 reduced STMN2 transcript with cryptic exon levels by 78%. A 500 nM treatment of a STMN2 AON with SEQ ID NO: 237 reduced STMN2 transcript with cryptic exon levels by 92%. Referring to FIG. 15B, STMN2-FL was decreased by 77% when treated with 500 nM TDP43 AON. A 50 nM treatment of a STMN2 AON with SEQ ID NO: 237 increased STMN-FL levels to 187% (rescued to 43%). A 200 nM treatment of a STMN2 AON with SEQ ID NO: 237 increased STMN-FL levels to 235% (rescued to 54%). A 500 nM treatment of a STMN2 AON with SEQ ID NO: 237 increased STMN-FL levels to 309% (rescued to 71%).

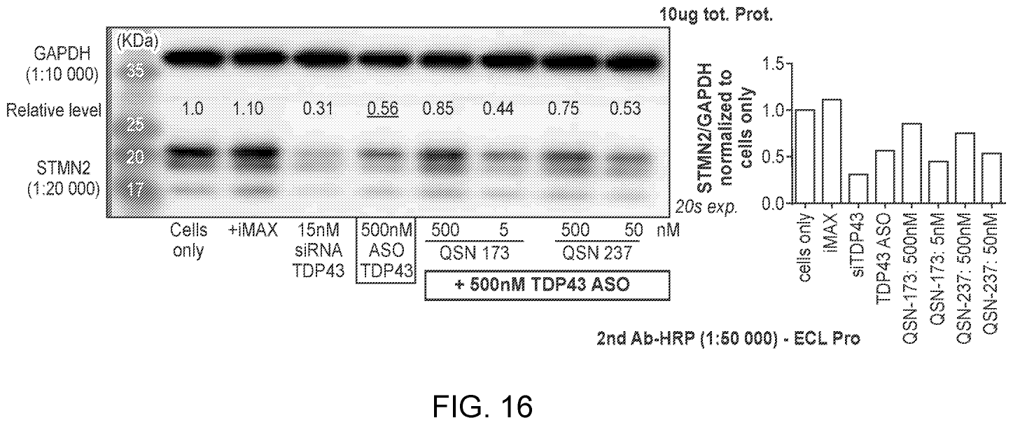

Referring to FIG. 16, STMN2-FL was decreased by 44% when treated with 500 nM TDP43 AON. A 500 nM treatment of a STMN2 AON with SEQ ID NO: 173 increased STMN-FL levels to 152%. A 500 nM treatment of a STMN2 AON with SEQ ID NO: 237 increased STMN-FL levels to 134%.

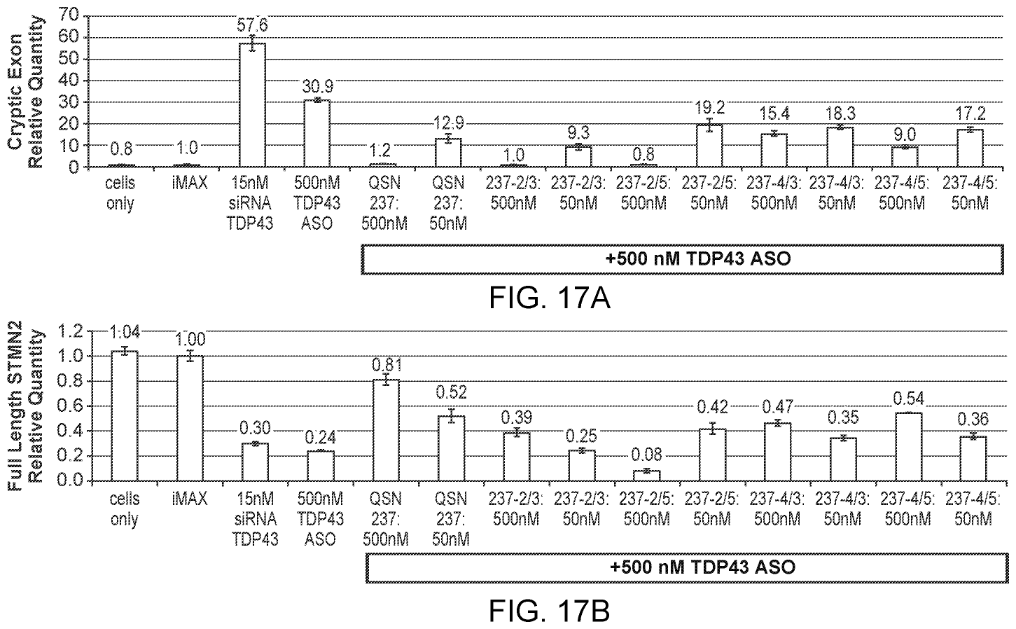

Referring to FIG. 17A, the quantity of STMN2 transcript with cryptic exon was increased more than 30-fold when treated with 500 nM TDP43 AON. A 500 nM treatment of a STMN2 AON with SEQ ID NO: 237 reduced STMN2 transcript with cryptic exon levels by 96%. A 500 nM treatment of a STMN2 AON with SEQ ID NO: 912 reduced STMN2 transcript with cryptic exon levels by 97%. A 500 nM treatment of a STMN2 AON with SEQ ID NO: 913 reduced STMN2 transcript with cryptic exon levels by 97%. A 500 nM treatment of a STMN2 AON with SEQ ID NO: 916 reduced STMN2 transcript with cryptic exon levels by 71%.

Referring to FIG. 17B, STMN2-FL was decreased by 76% when treated with 500 nM TDP43 AON. A 500 nM treatment of a STMN2 AON with SEQ ID NO: 237 increased STMN-FL levels to 338% (rescued to 81%). A 500 nM treatment of a STMN2 AON with SEQ ID NO: 912 increased STMN-FL levels to 163% (rescued to 39%). A 500 nM treatment of a STMN2 AON with SEQ ID NO: 915 increased STMN-FL levels to 196% (rescued to 47%). A 500 nM treatment of a STMN2 AON with SEQ ID NO: 916 increased STMN-FL levels to 225% (rescued to 54%).

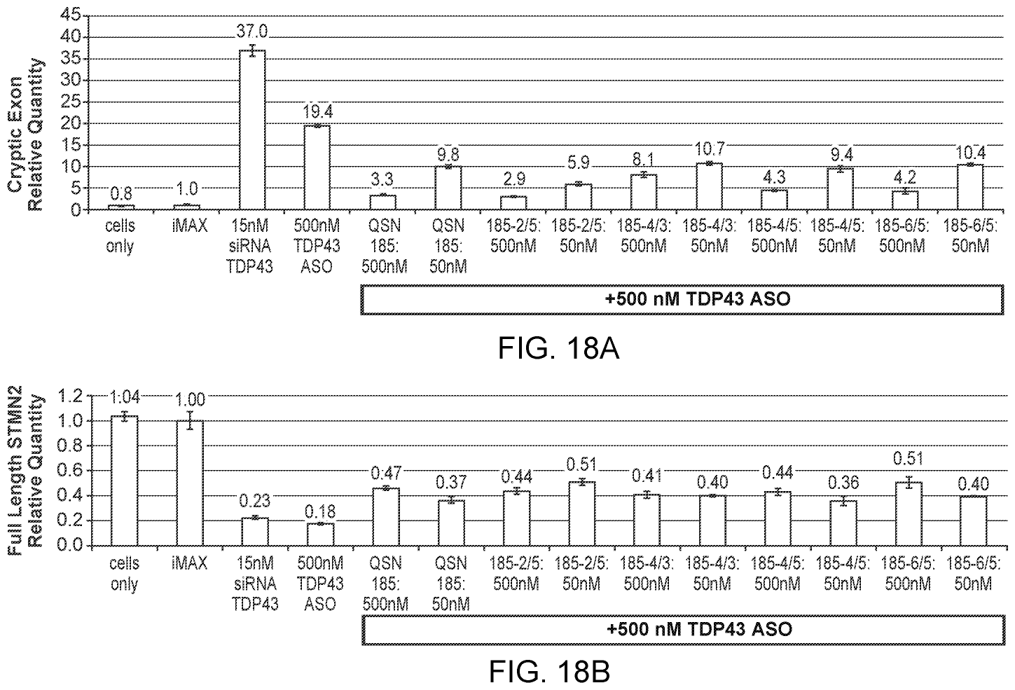

Referring to FIG. 18A, the quantity of STMN2 transcript with cryptic exon was increased more than 19-fold when treated with 500 nM TDP43 AON. A 500 nM treatment of a STMN2 AON with SEQ ID NO: 185 reduced STMN2 transcript with cryptic exon levels by 83%. A 500 nM treatment of a STMN2 AON with SEQ ID NO: 908 reduced STMN2 transcript with cryptic exon levels by 85%. A 500 nM treatment of a STMN2 AON with SEQ ID NO: 910 reduced STMN2 transcript with cryptic exon levels by 78%. A 500 nM treatment of a STMN2 AON with SEQ ID NO: 911 reduced STMN2 transcript with cryptic exon levels by 78%.

Referring to FIG. 18B, STMN2-FL was decreased by 82% when treated with 500 nM TDP43 AON. A 500 nM treatment of a STMN2 AON with SEQ ID NO: 185 increased STMN-FL levels to 261% (rescued to 47%). A 500 nM treatment of a STMN2 AON with SEQ ID NO: 908 increased STMN-FL levels to 244% (rescued to 44%). A 500 nM treatment of a STMN2 AON with SEQ ID NO: 909 increased STMN-FL levels to 228% (rescued to 41%). A 500 nM treatment of a STMN2 AON with SEQ ID NO: 910 increased STMN-FL levels to 244% (rescued to 44%). A 500 nM treatment of a STMN2 AON with SEQ ID NO: 911 increased STMN-FL levels to 283% (rescued to 51%).

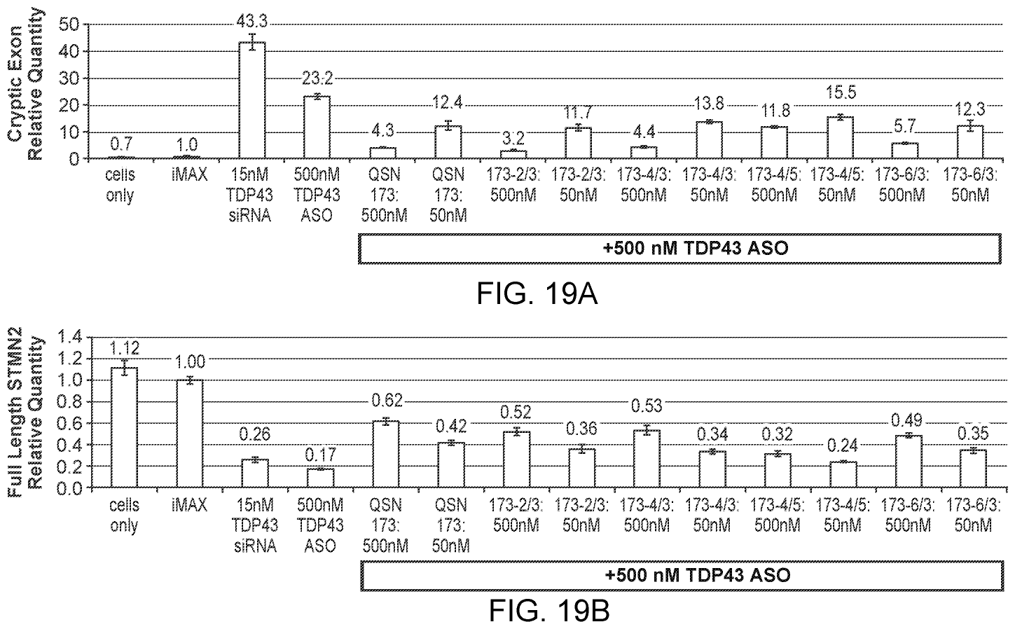

Referring to FIG. 19A, the quantity of STMN2 transcript with cryptic exon was increased more than 23-fold when treated with 500 nM TDP43 AON. A 500 nM treatment of a STMN2 AON with SEQ ID NO: 173 reduced STMN2 transcript with cryptic exon levels by 81%. A 500 nM treatment of a STMN2 AON with SEQ ID NO: 901 reduced STMN2 transcript with cryptic exon levels by 86%. A 500 nM treatment of a STMN2 AON with SEQ ID NO: 904 reduced STMN2 transcript with cryptic exon levels by 81%. A 500 nM treatment of a STMN2 AON with SEQ ID NO: 906 reduced STMN2 transcript with cryptic exon levels by 75%.

Referring to FIG. 19B, STMN2-FL was decreased by 83% when treated with 500 nM TDP43 AON. A 500 nM treatment of a STMN2 AON with SEQ ID NO: 173 increased STMN-FL levels to 365% (rescued to 62%). A 500 nM treatment of a STMN2 AON with SEQ ID NO: 901 increased STMN-FL levels to 306% (rescued to 52%). A 500 nM treatment of a STMN2 AON with SEQ ID NO: 904 increased STMN-FL levels to 312% (rescued to 53%). A 500 nM treatment of a STMN2 AON with SEQ ID NO: 905 increased STMN-FL levels to 188% (rescued to 32%). A 500 nM treatment of a STMN2 AON with SEQ ID NO: 906 increased STMN-FL levels to 288% (rescued to 49%).

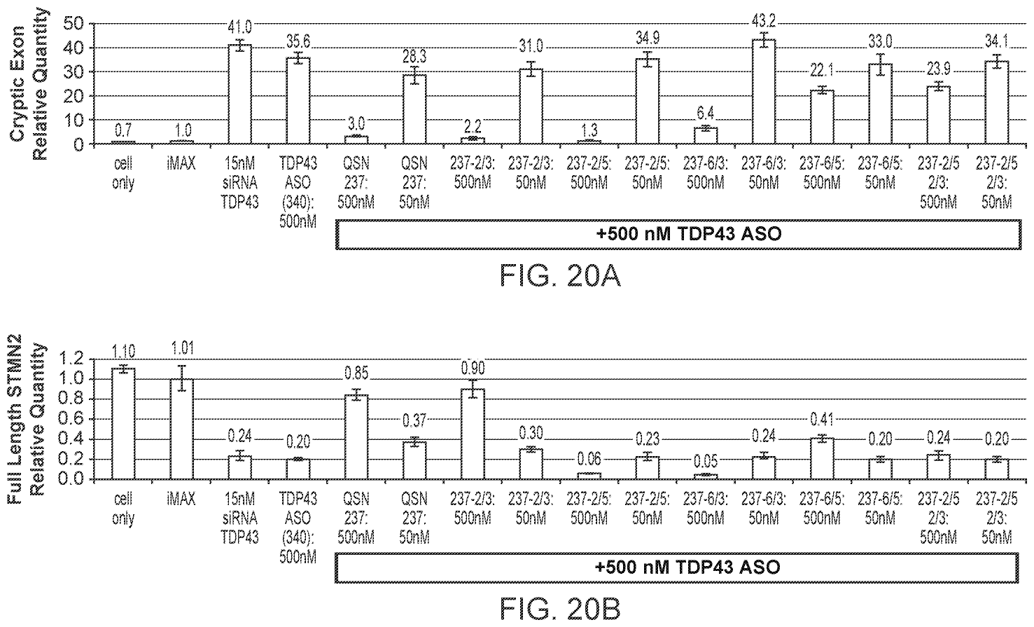

Referring to FIG. 20A, the quantity of STMN2 transcript with cryptic exon was increased more than 35-fold when treated with 500 nM TDP43 AON. A 500 nM treatment of a STMN2 AON with SEQ ID NO: 237 reduced STMN2 transcript with cryptic exon levels by 91%. A 500 nM treatment of a STMN2 AON with SEQ ID NO: 912 reduced STMN2 transcript with cryptic exon levels by 94%. A 500 nM treatment of a STMN2 AON with SEQ ID NO: 913 reduced STMN2 transcript with cryptic exon levels by 96%. A 500 nM treatment of a STMN2 AON with SEQ ID NO: 917 reduced STMN2 transcript with cryptic exon levels by 82%. A 500 nM treatment of a STMN2 AON with SEQ ID NO: 918 reduced STMN2 transcript with cryptic exon levels by 38%. A 500 nM treatment of a STMN2 AON with SEQ ID NO: 914 reduced STMN2 transcript with cryptic exon levels by 33%.

Referring to FIG. 20B, STMN2-FL was decreased by 80% when treated with 500 nM TDP43 AON. A 500 nM treatment of a STMN2 AON with SEQ ID NO: 237 increased STMN-FL levels to 425% (rescued to 85%). A 500 nM treatment of a STMN2 AON with SEQ ID NO: 912 increased STMN-FL levels to 450% (rescued to 90%). A 500 nM treatment of a STMN2 AON with SEQ ID NO: 918 increased STMN-FL levels to 205% (rescued to 41%).

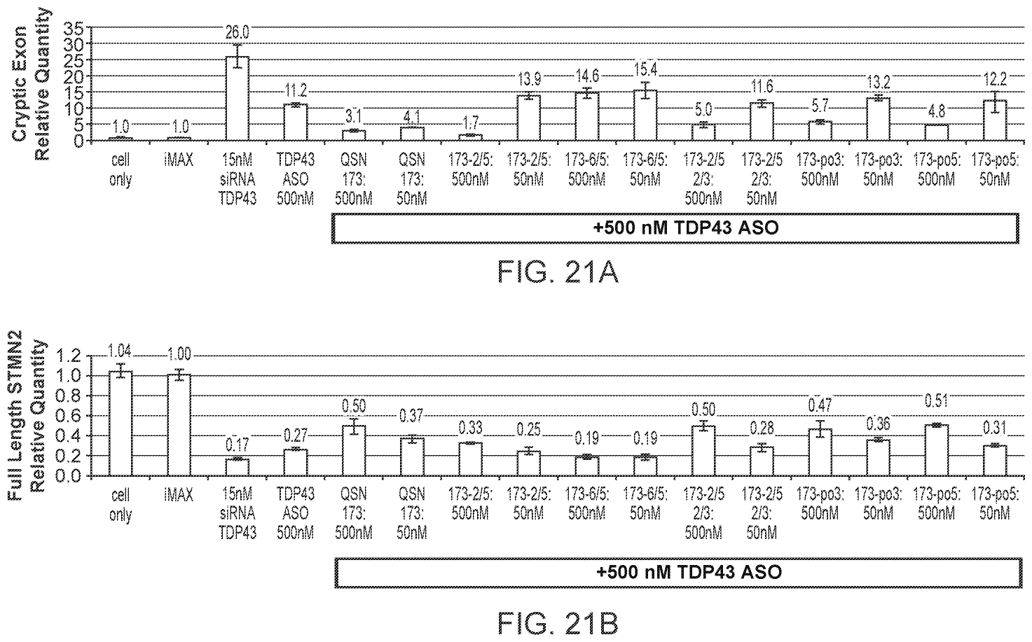

Referring to FIG. 21A, the quantity of STMN2 transcript with cryptic exon was increased more than 11-fold when treated with 500 nM TDP43 AON. A 500 nM treatment of a STMN2 AON with SEQ ID NO: 173 reduced STMN2 transcript with cryptic exon levels by 72%. A 500 nM treatment of a STMN2 AON with SEQ ID NO: 902 reduced STMN2 transcript with cryptic exon levels by 85%. A 500 nM treatment of a STMN2 AON with SEQ ID NO: 903 reduced STMN2 transcript with cryptic exon levels by 55%. A 500 nM treatment of a STMN2 AON with SEQ ID NO: 1417 reduced STMN2 transcript with cryptic exon levels by 49%. A 500 nM treatment of a STMN2 AON with SEQ ID NO: 1418 reduced STMN2 transcript with cryptic exon levels by 57%.

Referring to FIG. 21B, STMN2-FL was decreased by 73% when treated with 500 nM TDP43 AON. A 500 nM treatment of a STMN2 AON with SEQ ID NO: 173 increased STMN-FL levels by 85% (rescued to 50%). A 500 nM treatment of a STMN2 AON with SEQ ID NO: 903 increased STMN-FL levels by 85% (rescued to 50%). A 500 nM treatment of a STMN2 AON with SEQ ID NO: 1417 increased STMN-FL levels by 74% (rescued to 47%). A 500 nM treatment of a STMN2 AON with SEQ ID NO: 1418 increased STMN-FL levels by 89% (rescued to 51%).

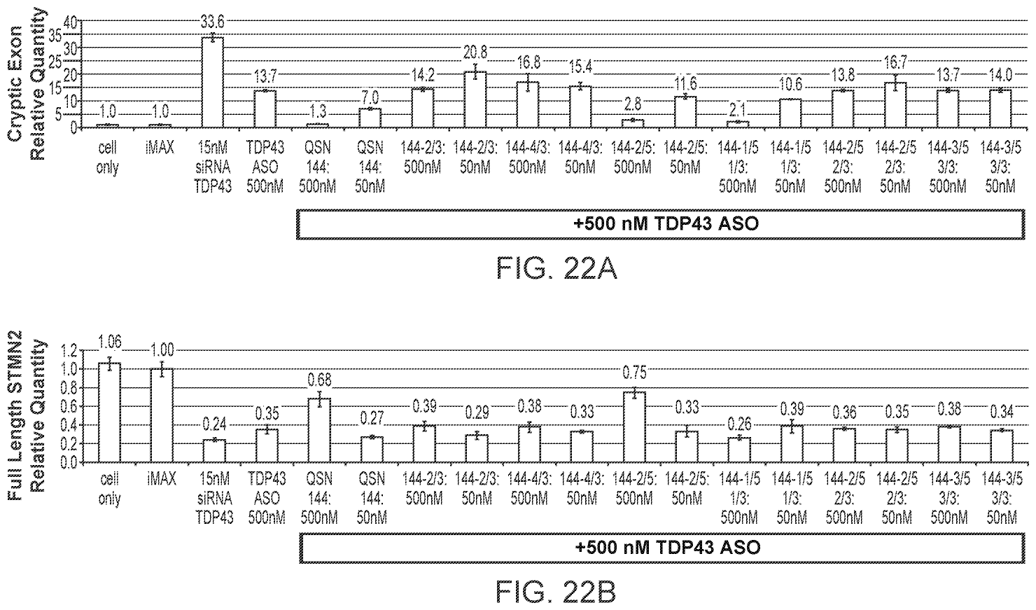

Referring to FIG. 22A, the quantity of STMN2 transcript with cryptic exon was increased more than 13-fold when treated with 500 nM TDP43 AON. A 500 nM treatment of a STMN2 AON with SEQ ID NO: 144 reduced STMN2 transcript with cryptic exon levels by 91%. A 500 nM treatment of a STMN2 AON with SEQ ID NO: 896 reduced STMN2 transcript with cryptic exon levels by 80%. A 500 nM treatment of a STMN2 AON with SEQ ID NO: 894 reduced STMN2 transcript with cryptic exon levels by 85%.

Referring to FIG. 22B, STMN2-FL was decreased by 65% when treated with 500 nM TDP43 AON. A 500 nM treatment of a STMN2 AON with SEQ ID NO: 144 increased STMN-FL levels by 94% (rescued to 68%). A 500 nM treatment of a STMN2 AON with SEQ ID NO: 896 increased STMN-FL levels by 114% (rescued to 75%).

### Example 4: Additional Experiments Demonstrating STMN2 Antisense Oligonucleotides Restore Full Length STMN2 and Reduce STMN2 Transcripts with Cryptic Exon in SY5Y Cells

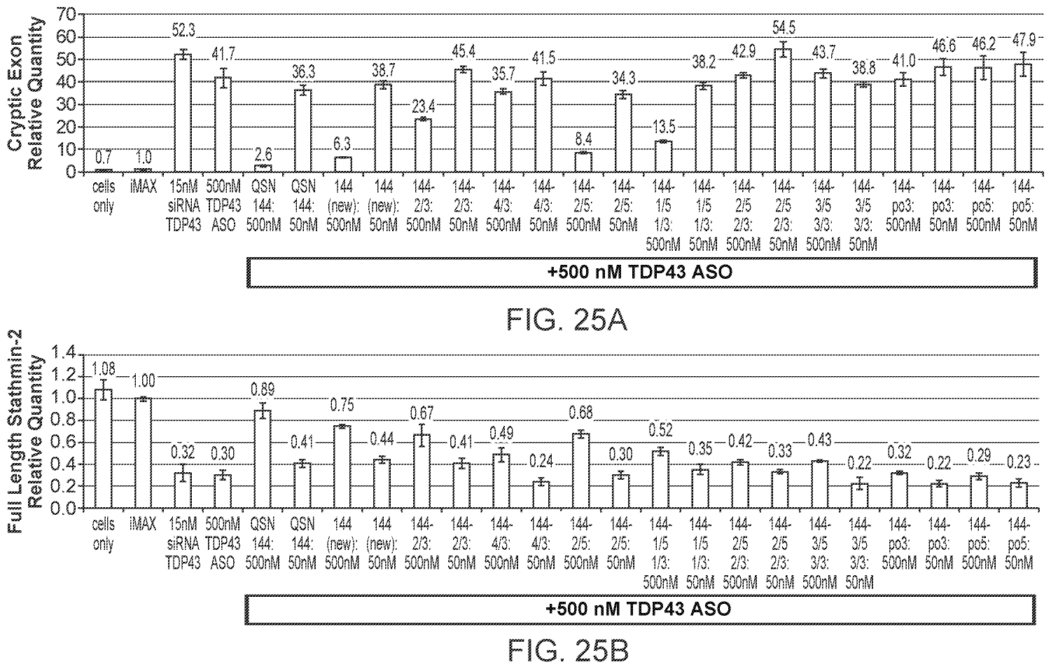

FIG. 25A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-144 STMN2 AONs and AON variants. FIG. 25B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-144 STMN2 AONs and AON variants. In particular, the STMN2 AONs and AON variants tested included: QSN-144 (SEQ ID NO: 144), QSN-144-2/3 (SEQ ID NO: 895), QSN-144-4/3 (SEQ ID NO: 899), QSN-144-2/5 (SEQ ID NO: 896), QSN-144-1/5 1/3 (SEQ ID NO: 894), QSN-144-2/5 2/3 (SEQ ID NO: 897), QSN-144-3/5 3/3 (SEQ ID NO: 898), QSN-144-po3 (SEQ ID NO: 1419), and QSN-144-po5 (SEQ ID NO: 1420).

Treatment with 500 nM TDP43 AON resulted in a 41.7 fold increase of STMN2 transcript with cryptic exon and a decrease of 70% STMN2-FL. The percentage decrease in STMN2 transcript with cryptic exon and the percentage increase in full length STMN2 in response to a 500 nM treatment of each respective STMN2 and AON variant is shown in Table 8.

TABLE 8
Effects of 500 nM treatment of QSN-144 STMN2 AON and QSN-144 AON variants.

| | | | Percentage | Relative | |
|---|---|---|---|---|---|
| | | Relative | Decrease | Full Length | Percentage |
| | SEQ | STMN2 | in STMN2 | STMN2 | Increase |
| STMN2 | ID | Cryptic Exon | transcript with | Quantity | in FL |
| AON | NO: | Quantity | cryptic exon | (“rescued to”) | STMN2 |
| 144-2/3 | 895 | 23.4 | 43.9 | 0.67 | 223.3 |
| 144-4/3 | 899 | 35.7 | 14.4 | 0.49 | 163.3 |
| 144-2/5 | 896 | 8.4 | 79.9 | 0.86 | 286.7 |
| 144-1/5-1/3 | 894 | 13.5 | 67.6 | 0.52 | 173.3 |
| 144-2/5-2/3 | 897 | 42.9 | −2.9 | 0.42 | 140.0 |
| 144-3/5-3/3 | 898 | 43.7 | −4.8 | 0.43 | 143.3 |
| 144-PO3 | 1419 | 41 | 1.7 | 0.32 | 106.7 |
| 144-PO5 | 1420 | 46.2 | −10.8 | 0.29 | 96.7 |

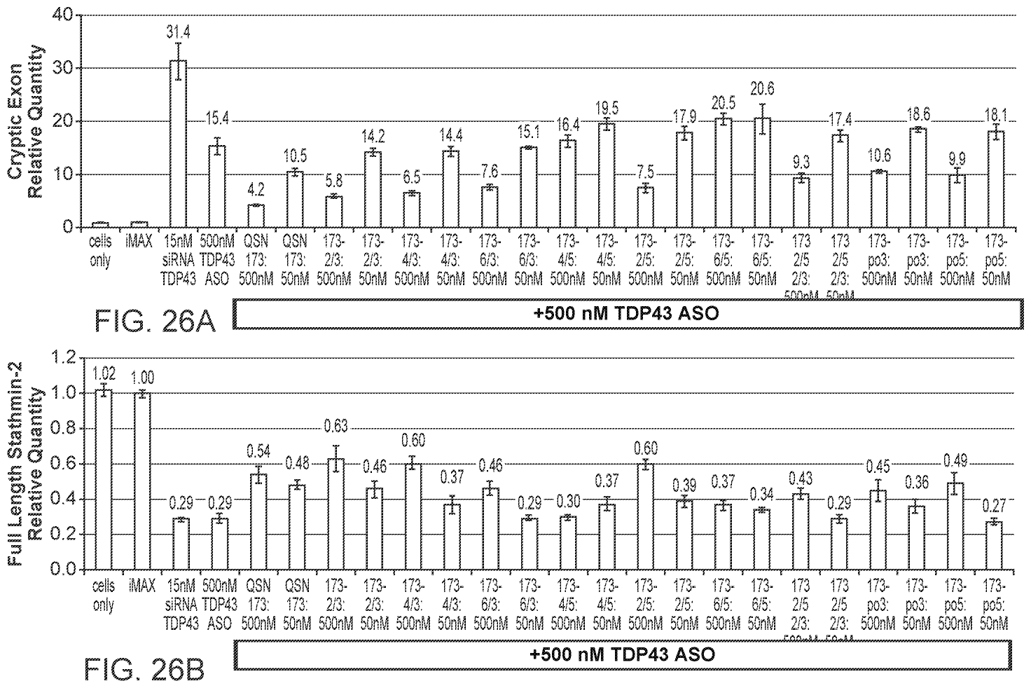

FIG. 26A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-173 STMN2 AONs and AON variants. FIG. 26B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-173 STMN2 AONs and AON variants. In particular, the STMN2 AONs and AON variants tested included: QSN-173 (SEQ ID NO: 173), QSN-173-2/3 (SEQ ID NO: 901), QSN-173-4/3 (SEQ ID NO: 904), QSN-173-6/3 (SEQ ID NO: 906), QSN-173-4/5 (SEQ ID NO: 905), QSN-173-2/5 (SEQ ID NO: 902), QSN-173-6/5 (SEQ ID NO: 907), QSN-173-2/5 2/3 (SEQ ID NO: 903), QSN-173-po3 (SEQ ID NO: 1417), and QSN-173-po5 (SEQ ID NO: 1418). Treatment with 500 nM TDP43 AON resulted in a 15.4 fold increase of STMN2 transcript with cryptic exon and a decrease of 71% STMN2-FL. The percentage decrease in STMN2 transcript with cryptic exon and the percentage increase in full length STMN2 in response to a 500 nM treatment of each respective STMN2 and AON variant is shown in Table 9.

TABLE 9
Effects of 500 nM treatment of QSN-173 STMN2 AON and QSN-173 AON variants.

| | | | Percentage | Relative | |
|---|---|---|---|---|---|
| | | Relative | Decrease | Full Length | Percentage |
| | SEQ | STMN2 | in STMN2 | STMN2 | Increase |
| STMN2 | ID | Cryptic Exon | transcript with | Quantity | in FL |
| AON | NO: | Quantity | cryptic exon | (“rescued to”) | STMN2 |
| 173-2/3 | 901 | 5.8 | 62.3 | 0.83 | 286.2 |
| 173-4/3 | 904 | 6.5 | 57.8 | 0.6 | 206.9 |
| 173-6/3 | 906 | 7.6 | 50.6 | 0.46 | 158.6 |
| 173-4/5 | 905 | 16.4 | −6.5 | 0.3 | 103.4 |
| 173-2/5 | 902 | 7.5 | 51.3 | 0.6 | 206.9 |
| 173-6/5 | 907 | 20.5 | −33.1 | 0.37 | 127.6 |
| 173-2/5-2/3 | 903 | 9.3 | 39.6 | 0.43 | 148.3 |
| 173-PO3 | 1417 | 10.5 | 31.8 | 0.45 | 155.2 |
| 173-PO5 | 1418 | 9.9 | 35.7 | 0.49 | 169.0 |

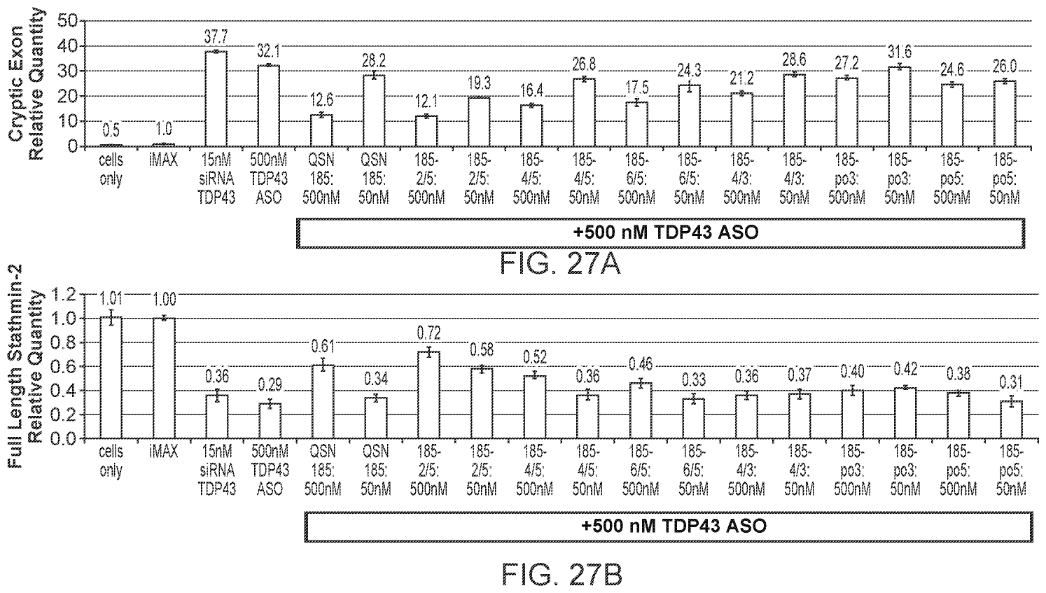

FIG. 27A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-185 STMN2 AONs and AON variants. FIG. 27B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-185 STMN2 AONs and AON variants. In particular, the STMN2 AONs and AON variants tested included: QSN-185 (SEQ ID NO: 185), QSN-185-2/5 (SEQ ID NO: 908), QSN-185-4/5 (SEQ ID NO: 910), QSN-185-6/5 (SEQ ID NO: 911), QSN-185-4/3 (SEQ ID NO: 909), QSN-185-po3 (SEQ ID NO: 1421), and QSN-185-po5 (SEQ ID NO: 1422). Treatment with 500 nM TDP43 AON resulted in a 32.1 fold increase of STMN2 transcript with cryptic exon and a decrease of 71% STMN2-FL. The percentage decrease in STMN2 transcript with cryptic exon and the percentage increase in full length STMN2 in response to a 500 nM treatment of each respective STMN2 and AON variant is shown in Table 10.

TABLE 10
Effects of 500 nM treatment of QSN-185 STMN2 AON and QSN-185 AON variants.

| | | | Percentage | Relative | |
|---|---|---|---|---|---|
| | | Relative | Decrease | Full Length | Percentage |
| | SEQ | STMN2 | in STMN2 | STMN2 | Increase |
| STMN2 | ID | Cryptic Exon | transcript with | Quantity | in FL |
| AON | NO: | Quantity | cryptic exon | (“rescued to”) | STMN2 |
| 185-2/5 | 908 | 12.1 | 62.3 | 0.72 | 248.3 |
| 185-4/5 | 910 | 16.4 | 48.9 | 0.52 | 179.3 |
| 185-6/5 | 911 | 17.5 | 45.5 | 0.46 | 158.6 |
| 185-4/3 | 909 | 21.2 | 34.0 | 0.36 | 124.1 |
| 185-PO3 | 1421 | 27.2 | 15.3 | 0.4 | 137.9 |
| 185-PO5 | 1422 | 24.6 | 23.4 | 0.38 | 131.0 |

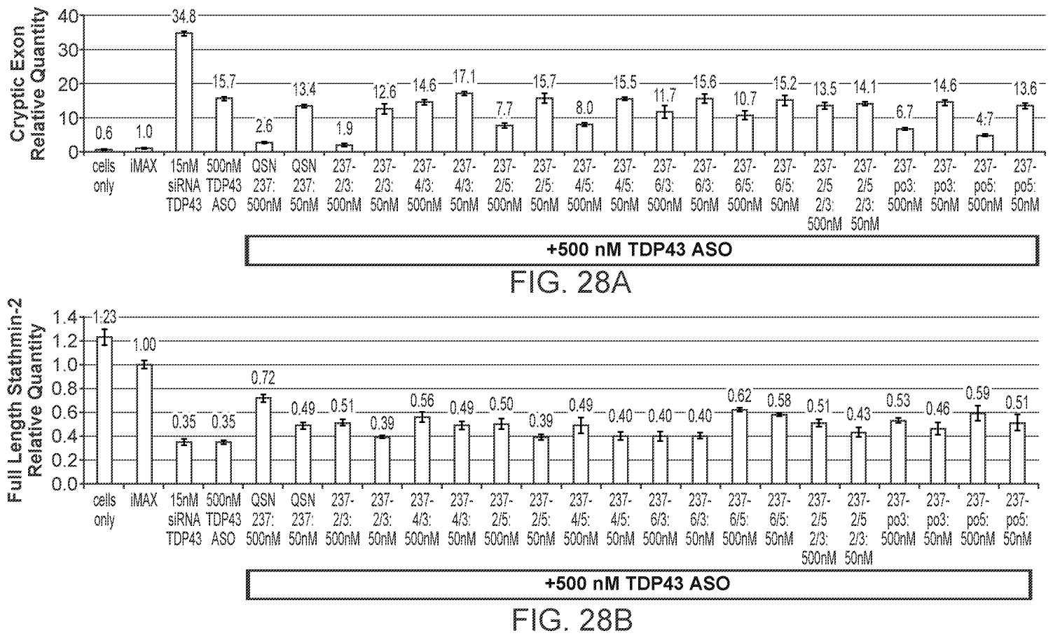

FIG. 28A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different QSN-237 STMN2 AONs and AON variants. FIG. 28B is a bar graph showing the results of RT-qPCR analysis of TDP43 and STMN2 full-length mRNA levels in the presence of TDP43 siRNA and TDP43 antisense, and restoration of the full-length STMN2 transcript using different QSN-237 STMN2 AONs and AON variants. In particular, the STMN2 AONs and AON variants tested included: QSN-237 (SEQ ID NO: 237), QSN-237-2/3 (SEQ ID NO: 912), QSN-237-4/3 (SEQ ID NO: 915), QSN-237-2/5 (SEQ ID NO: 913), QSN-237-4/5 (SEQ ID NO: 916), QSN-237-6/3 (SEQ ID NO: 917), QSN-237-6/5 (SEQ ID NO: 918), QSN-237-2/5 2/3 (SEQ ID NO: 914), QSN-237-po3 (SEQ ID NO: 1423), and QSN-237-po5 (SEQ ID NO: 1424). Treatment with 500 nM TDP43 AON resulted in a 15.7 fold increase of STMN2 transcript with cryptic exon and a decrease of 65% STMN2-FL. The percentage decrease in STMN2 transcript with cryptic exon and the percentage increase in full length STMN2 in response to a 500 nM treatment of each respective STMN2 and AON variant is shown in Table 11.

TABLE 11
Effects of 500 nM treatment of QSN-237 STMN2 AON and QSN-237 AON variants.

| | | | Percentage | Relative | |
|---|---|---|---|---|---|
| | | Relative | Decrease | Full Length | Percentage |
| | SEQ | STMN2 | in STMN2 | STMN2 | Increase |
| STMN2 | ID | Cryptic Exon | transcript with | Quantity | in FL |
| AON | NO: | Quantity | cryptic exon | (“rescued to”) | STMN2 |
| 237-2/3 | 912 | 1.9 | 87.9 | 0.51 | 145.7 |
| 237-4/3 | 915 | 14.6 | 7.0 | 0.56 | 160.0 |
| 237-2/5 | 913 | 7.7 | 51.0 | 0.5 | 142.9 |
| 237-4/5 | 916 | 8 | 49.0 | 0.49 | 140.0 |
| 237-6/3 | 917 | 11.7 | 25.5 | 0.4 | 114.3 |
| 237-6/5 | 918 | 10.7 | 31.8 | 0.62 | 177.1 |
| 237-2/5-2/3 | 914 | 13.5 | 14.0 | 0.51 | 145.7 |
| 237-PO3 | 1423 | 6.7 | 57.3 | 0.53 | 151.4 |
| 237-PO5 | 1424 | 4.7 | 70.1 | 0.59 | 168.6 |

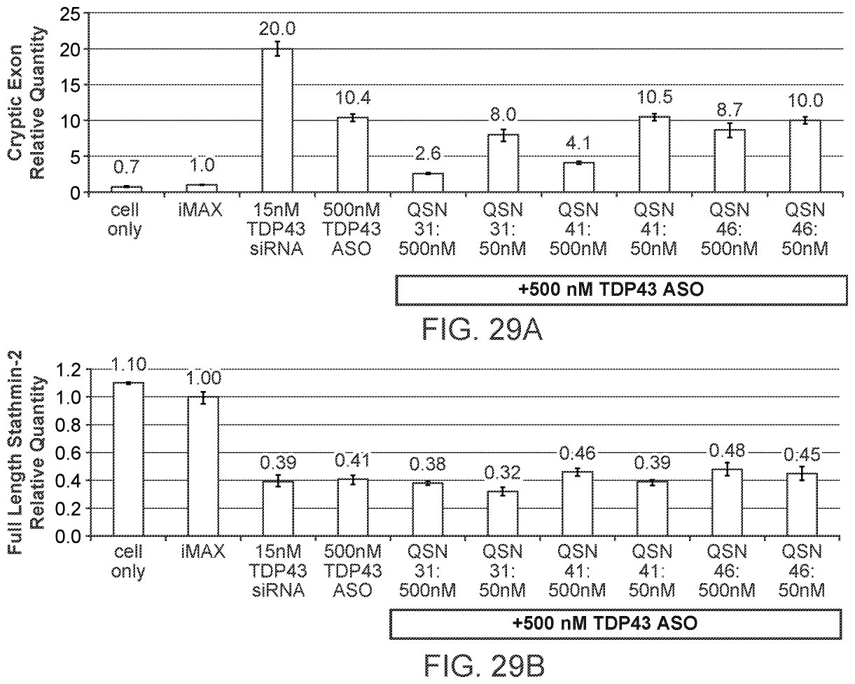

FIG. 29A is a bar graph showing the results of RT-qPCR analysis of STMN2 transcript with cryptic exon mRNA levels in the presence of TDP43 siRNA and TDP43 antisense, and reduction of the STMN2 transcript with cryptic exon mRNA levels using different STMN2 AONs (QSN-31, QSN-41, and QSN-46). FIG. 29B is a bar graph showing the results of RT-qPCR analysis of TDP43 and STMN2 full-length mRNA levels in the presence of TDP43 siRNA and TDP43 antisense, and restoration of the full-length STMN2 transcript using different STMN2 AONs (QSN-31, QSN-41, and QSN-46). In particular, the STMN2 AONs and AON variants tested included: QSN-31 (SEQ ID NO: 31), QSN-41 (SEQ ID NO: 41), and QSN-46 (SEQ ID NO: 46). Treatment with 500 nM TDP43 AON resulted in a 10.4 fold increase of STMN2 transcript with cryptic exon and a decrease of 59% STMN2-FL. The percentage decrease in STMN2 transcript with cryptic exon and the percentage increase in full length STMN2 in response to a 500 nM treatment of each respective STMN2 and AON variant is shown in Table 12.

TABLE 12
Effects of 500 nM treatment of QSN-31, QSN-41, and QSN-46 STMN2 AONs.

| | | | Percentage | Relative | |
|---|---|---|---|---|---|
| | | Relative | Decrease | Full Length | Percentage |
| | SEQ | STMN2 | in STMN2 | STMN2 | Increase |
| STMN2 | ID | Cryptic Exon | transcript with | Quantity | in FL |
| AON | NO: | Quantity | cryptic exon | (“rescued to”) | STMN2 |
| 31 | 31 | 2.6 | 75.0 | 0.38 | 108.6 |
| 41 | 41 | 4.1 | 60.6 | 0.46 | 131.4 |
| 46 | 46 | 8.7 | 16.3 | 0.48 | 137.1 |

### Example 5: Dose Response Restoration of Full Length STMN2 mRNA and STMN2 Protein Using Stathmin-2 Cryptic Splicing Modulator

The experiment was performed as previously described in human neuroblastoma SY5Y cells. The cells were plated in 6-well or 96-well plates and cultured to 80% confluency. TDP-43 expression in cells were knocked down using an AON to TDP43 to express the cryptic exon, thus preventing transcription of full-length STMN2 (STMN2-FL) product. Cells were additionally co-transfected with a STMN2 ASO (specifically, QSN-237-2/3 (SEQ ID NO: 912)) at varying doses (5 nM, 50 nM, 100 nM, 200 nM, and 500 nM). RNA and protein were isolated for QPCR and western blot assays.

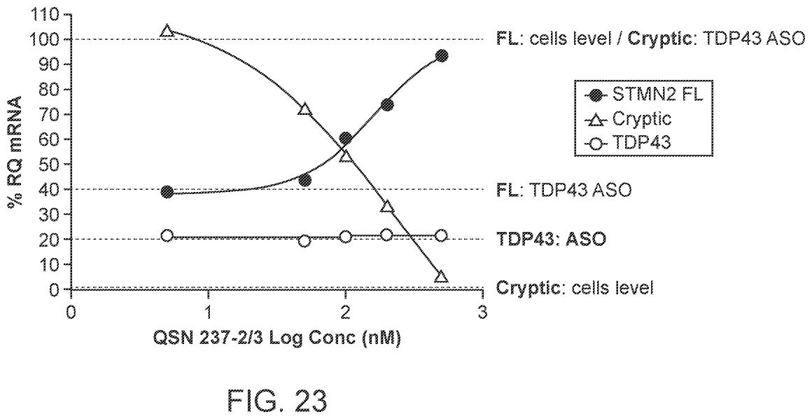

FIG. 23 shows the dose response curve illustrating increasing restoration of full length STMN2 transcript with increasing concentrations of STMN2 AON. Generally, increasing concentrations of STMN2 AON increased full length STMN2 mRNA, decreased cryptic exon levels. Specifically, a 5 nM treatment of the STMN2 ASO resulted in —40% restoration of full length STMN2 transcript. A 500 nM treatment of the STMN2 ASO resulted in nearly 100% restoration of full length STMN2 transcript. Additionally, the 500 nM treatment of the STMN2 ASO resulted in the significant reduction (close to 0%) of cryptic exon.

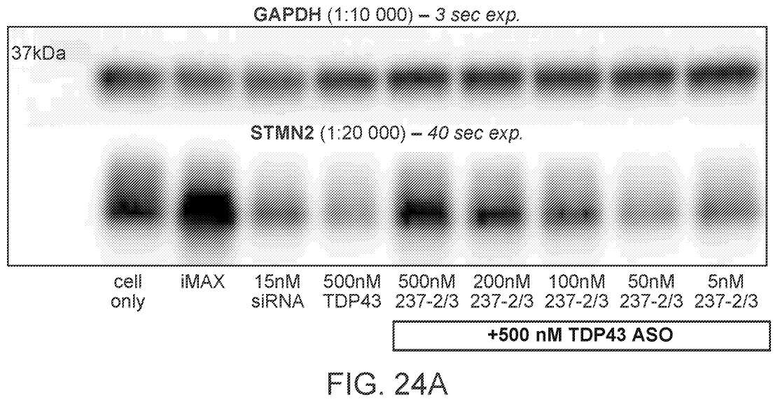

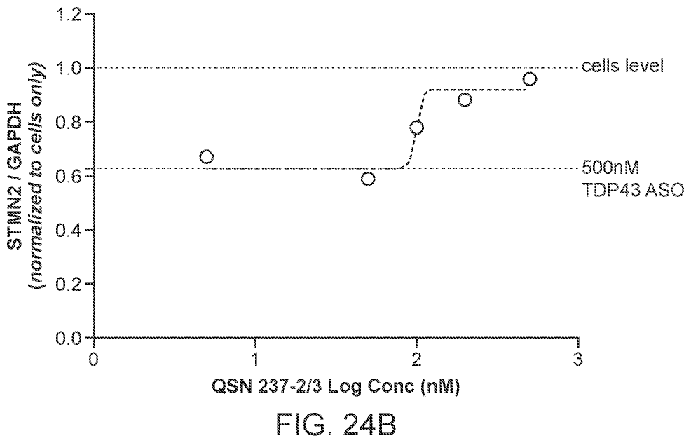

FIG. 24A shows a Western blot assay demonstrating the qualitative increase of full length STMN2 protein in response to higher concentrations of STMN2 AON. FIG. 24B shows the quantitated levels of full length STMN2 protein normalized to GAPDH in response to different concentrations of STMN2 AON. Generally, both FIGS. 24A and 24B show that increasing concentrations of the STMN2 AON resulted in increasing concentrations of full length STMN2 protein. Specifically, as shown in FIG. 24B, lower concentrations (5 nM and 50 nM) of the STMN2 AON resulted in full length STMN2 protein concentrations that were ˜60% of the control group (cell only). Notably, the 500 nM treatment of the STMN2 ASO resulted in nearly 100% restoration of the full length STMN2 protein (in comparison to the cell only control group).

### Example 6: Chemotherapy Induced Neuropathy as an Indication that can be Targeted by a Stathmin-2 Cryptic Splicing Modulator

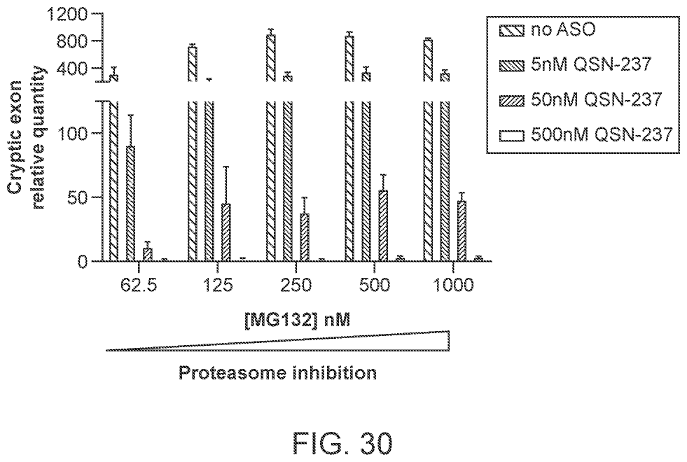

Referring to FIG. 30, it illustrates a bar graph showing reversal of cryptic exon induction using QSN-237 STMN2 antisense oligonucleotide (SEQ ID NO: 237) even in view of increasing proteasome inhibition. As a control, cells that were treated with endoporter alone (no AON) and then subsequently treated with MG132 (across all concentrations of MG132) demonstrated high levels of cryptic exon. This is indicative of TDP-43 pathology induced by proteasome inhibition in human motor neurons. MG132 causes TDP43 mislocalization leading to STMN2 mis-splicing and increased cryptic exon expression. The addition of QSN-237 (SEQ ID NO: 237) antisense oligonucleotide reverses cryptic exon induction with high potency (IC50 <5 nM). As shown in FIG. 30, increasing concentrations of QSN-237 (ranging from 5 nM up to 500 nM) significantly reduces the cryptic exon relative quantity.

In totality, this data establishes that the QSN-237 antisense oligonucleotide (SEQ ID NO: 237) protects against proteotoxic stress induction of cryptic exon expression. This is applicable in settings where neurons are to be protected from proteotoxic stress as a result of other therapies such as chemotherapeutics.

### Example 7: STMN2 Antisense Oligonucleotides Restore Full Length STMN2 and Reduce STMN2 Transcripts with Cryptic Exon in Human Motor Neurons

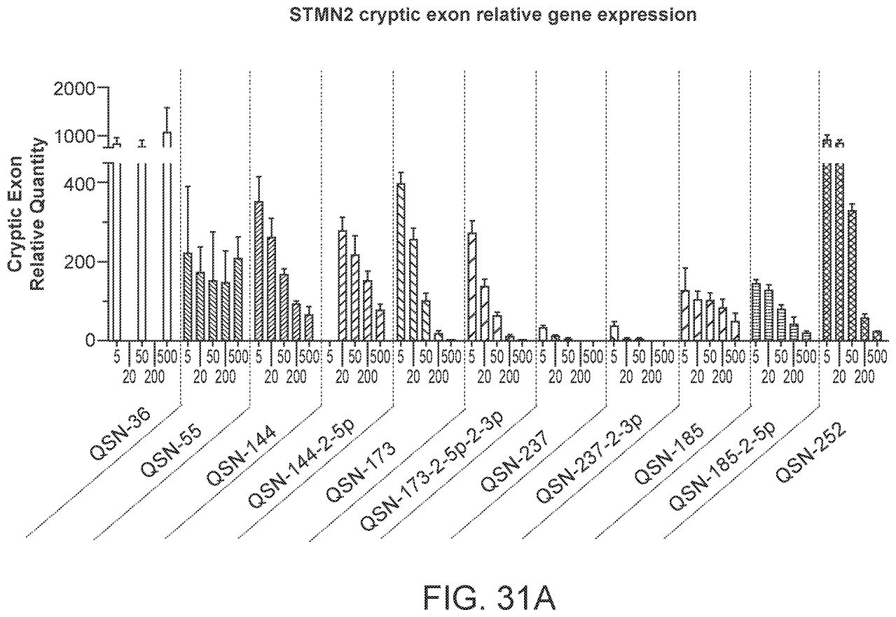

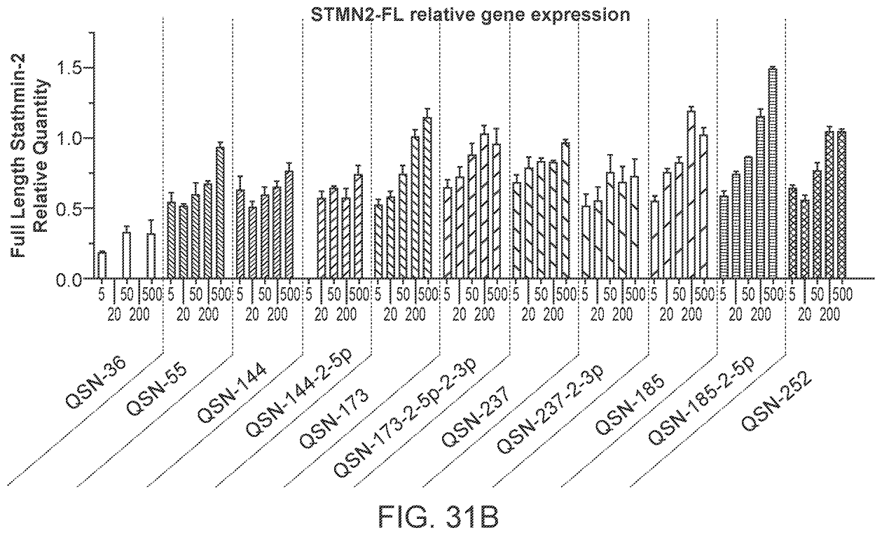

FIG. 31A and FIG. 31B show bar graphs showing the results of RT-qPCR analysis of STMN2 transcript with cryptic exon mRNA levels and STMN2 full-length mRNA levels, which demonstrate reduction of the STMN2 transcript with cryptic exon mRNA levels and restoration of the full-length STMN2 transcript using different STMN2 AONs and AON variants. In particular, the STMN2 AONs and AON variants tested included: QSN-36 (SEQ ID NO: 36), QSN-55 (SEQ ID NO: 55), QSN-144 (SEQ ID NO: 144), QSN-144-2/5 (SEQ ID NO: 896), QSN-173 (SEQ ID NO: 173), QSN-173-2/5-2/3 (SEQ ID NO: 903), QSN-237 (SEQ ID NO: 237), QSN-237-2/3 (SEQ ID NO: 912), QSN 185 (SEQ ID NO: 185), QSN-185-2/5 (SEQ ID NO: 908), and QSN-252 (SEQ ID NO: 252).

Table 13 shows the dose-dependent effect (percentage decrease) of STMN2 AONs on levels of expression of STMN2 with cryptic exon. Additionally, Table 14 shows the dose-dependent effect of STMN2 AONs on restoration of levels of full length STMN2 transcript.

TABLE 13
Dose dependent effect of STMN2 AONs on expression levels of STMN2 with cryptic exon.
Values are shown as percentage decrease of expression levels of STMN2 with cryptic exon
relative to corresponding value derived from 500 nM TDP43 AON treated cells.

| | | | | QSN-144- | | QSN-173- |
|---|---|---|---|---|---|---|
| Dose | QSN-36 | QSN-55 | QSN-144 | 2-5p | QSN-173 | 2-5p-2-3p |
| 5 | −6 | 17 | 87 | | 85 | 89 |
| 20 | | 35 | 90 | 90 | 91 | 94 |
| 50 | 3 | 43 | 94 | 92 | 96 | 97 |
| 200 | | 45 | 97 | 94 | 99 | 100 |
| 500 | −37 | 21 | 98 | 97 | 100 | 100 |

| Dose | QSN-237 | QSN-237-2-3p | QSN-185 | QSN-185-2-5p | QSN-252 |
|---|---|---|---|---|---|
| 5 | 99 | 98 | 11 | −1 | 22 |
| 20 | 99 | 100 | 28 | 10 | 28 |
| 50 | 100 | 100 | 28 | 44 | 71 |
| 200 | 100 | 100 | 42 | 71 | 95 |
| 500 | 100 | 100 | 66 | 86 | 98 |

TABLE 14
Dose dependent effect of STMN2 AONs on
expression levels of full length STMN2 transcript.

| | QSN-36 | | QSN-55 | | QSN-144 | | QSN-144-2-5p | |
|---|---|---|---|---|---|---|---|---|
| | Relative | Percent | Relative | Percent | Relative | Percent | Relative | Percent |
| | Quantity | Increase | Quantity | Increase | Quantity | Increase | Quantity | Increase |
| | (“rescued | of FL | (“rescued | of FL | (“rescued | of FL | (“rescued | of FL |
| Dose | to”) | STMN2 | to”) | STMN2 | to”) | STMN2 | to”) | STMN2 |
| 5 | 0.19 | 69 | 0.54 | 136 | 0.63 | 524 | | |
| 20 | | | 0.51 | 129 | 0.51 | 423 | 0.57 | 477 |
| 50 | 0.33 | 122 | 0.60 | 149 | 0.60 | 497 | 0.64 | 536 |
| 200 | | | 0.67 | 168 | 0.65 | 543 | 0.57 | 477 |
| 500 | 0.32 | 119 | 0.93 | 234 | 0.77 | 639 | 0.74 | 616 |
| | QSN-173 | | QSN-173-2-5p-2-3p | | QSN-237 | | QSN-237-2-3p | |
| | Relative | Percent | Relative | Percent | Relative | Percent | Relative | Percent |
| | Quantity | Increase | Quantity | Increase | Quantity | Increase | Quantity | Increase |
| Dose | (“rescued | of FL | (“rescued | of FL | (“rescued | of FL | (“rescued | of FL |
| | to”) | STMN2 | to”) | STMN2 | to”) | STMN2 | to”) | STMN2 |
| 5 | 0.52 | 437 | 0.65 | 499 | 0.68 | 525 | 0.52 | 397 |
| 20 | 0.58 | 483 | 0.72 | 556 | 0.79 | 606 | 0.55 | 426 |
| 50 | 0.74 | 619 | 0.88 | 677 | 0.83 | 640 | 0.76 | 581 |
| 200 | 1.01 | 840 | 1.03 | 791 | 0.83 | 636 | 0.68 | 527 |
| 500 | 1.15 | 954 | 0.96 | 736 | 0.97 | 743 | 0.73 | 560 |

| | QSN-185 | | QSN-185-2-5p | | QSN-252 | |
|---|---|---|---|---|---|---|
| | Relative | Percent | Relative | Percent | Relative | Percent |
| | Quantity | Increase | Quantity | Increase | Quantity | Increase |
| | (“rescued | of FL | (″rescued | of FL | (“rescued | of FL |
| Dose | to”) | STMN2 | to″) | STMN2 | to”) | STMN2 |
| 5 | 0.55 | 184 | 0.59 | 196 | 0.64 | 121 |
| 20 | 0.75 | 252 | 0.74 | 248 | 0.55 | 104 |
| 50 | 0.83 | 275 | 0.86 | 288 | 0.77 | 144 |
| 200 | 1.19 | 396 | 1.15 | 384 | 1.04 | 197 |
| 500 | 1.02 | 340 | 1.49 | 498 | 1.04 | 197 |

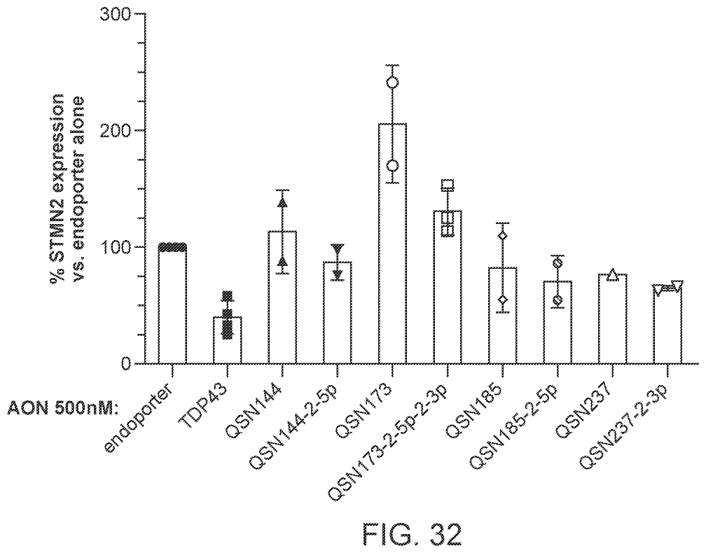

FIG. 32 is a bar graph showing the results of a western blot analysis of STMN2 protein levels, which demonstrates, which demonstrates restoration of the full-length STMN2 protein using different STMN2 AONs and AON variants. In particular, the STMN2 AONs and AON variants tested included: QSN-144 (SEQ ID NO: 144), QSN-144-2/5 (SEQ ID NO: 896), QSN-173 (SEQ ID NO: 173), QSN-173-2/5-2/3 (SEQ ID NO: 903), QSN-185 (SEQ ID NO: 185), QSN-185-2/5 (SEQ ID NO: 908), QSN-237 (SEQ ID NO: 237), and QSN-237-2/3 (SEQ ID NO: 912).

Table 15 below shows the expression levels of STMN2 protein in relation to control groups (endoporter and TDP43 ASO). Each of the STMN2 AONs and AON variants increased expression levels of STMN2 protein in relation to TDP43 ASO. In some cases, STMN2 AONs (e.g., QSN-144 and QSN-173) and AON variants (e.g., QSN-173-2/5-2/3) restored expression levels of STMN2 protein to levels above the endoporter control.

TABLE 15
Full length STMN2 expression of
human motor neurons treated with
STMN2 AONs or AON variants.

| | percent of |
|---|---|
| Group | endoporter |
| Endoporter | 100 |
| TDP43 ASO | 40 |
| QSN-144 | 113 |
| QSN-144-2/5 | 87 |
| QSN-173 | 206 |
| QSN-173-2/5-2/3 | 131 |
| QSN-185 | 82 |
| QSN-185-2/5 | 71 |
| QSN-237 | 76 |
| QSN-237-2/3 | 65 |

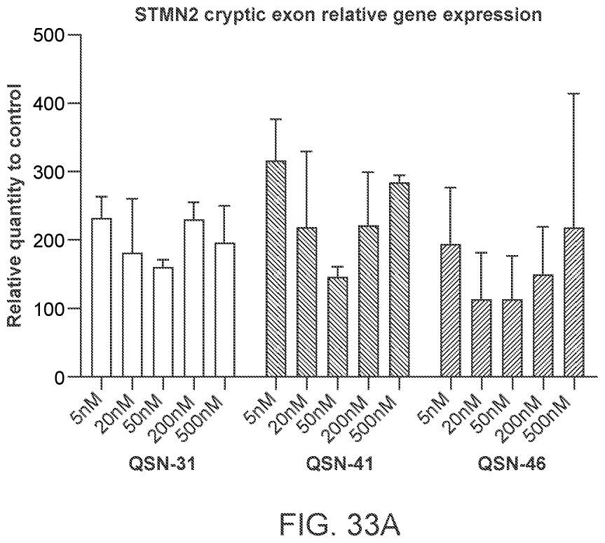

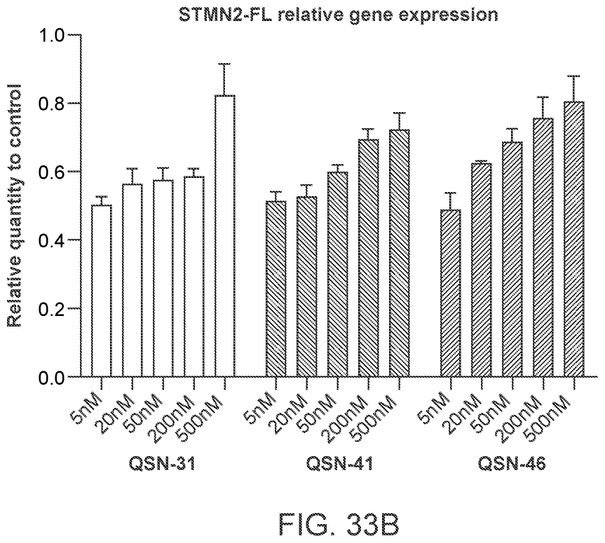

FIG. 33A is a bar graph showing the results of RT-qPCR analysis of STMN2 transcript with cryptic exon mRNA expression in human motor neurons in response to treatment using different STMN2 AONs. FIG. 33B is a bar graph showing the results of RT-qPCR analysis of STMN2 full-length mRNA levels in response to treatment using different STMN2 AONs. In particular, the STMN2 AONs tested included: QSN-31 (SEQ ID NO: 31), QSN-41 (SEQ ID NO: 41), and QSN-46 (SEQ ID NO: 46).

Table 16 shows the dose-dependent effect (percentage decrease) of STMN2 AONs on levels of expression of STMN2 with cryptic exon. Additionally, Table 17 shows the dose-dependent effect of STMN2 AONs on restoration of levels of full length STMN2 transcript.

TABLE 16
Dose dependent effect of
STMN2 AONs on expression levels of
STMN2 with cryptic exon. Values are shown as
percentage decrease of expression levels of STMN2
with cryptic exon relative to corresponding value
derived from 500 nM TDP43 AON treated cells.

| Dose | QSN-31 | QSN-41 | QSN-46 |
|---|---|---|---|
| 5 | −62 | −19 | 27 |
| 20 | −26 | 18 | 57 |
| 50 | −12 | 45 | 57 |
| 200 | −60 | 17 | 44 |
| 500 | −36 | −7 | 18 |

TABLE 17
Dose dependent effect of STMN2 AONs on expression
levels of full length STMN2 transcript.

| | QSN-31 | | QSN-41 | | QSN-46 | |
|---|---|---|---|---|---|---|
| | Relative | Percent | Relative | Percent | Relative | Percent |
| | Quantity | Increase | Quantity | Increase | Quantity | Increase |
| | (“rescued | of FL | (“rescued | of FL | (“rescued | of FL |
| Dose | to”) | STMN2 | to”) | STMN2 | to”) | STMN2 |
| 5 | 0.50 | 167 | 0.51 | 127 | 0.49 | 121 |
| 20 | 0.56 | 187 | 0.53 | 132 | 0.62 | 156 |
| 50 | 0.57 | 191 | 0.60 | 149 | 0.69 | 171 |
| 200 | 0.58 | 195 | 0.69 | 173 | 0.75 | 189 |
| 500 | 0.82 | 274 | 0.72 | 180 | 0.80 | 201 |

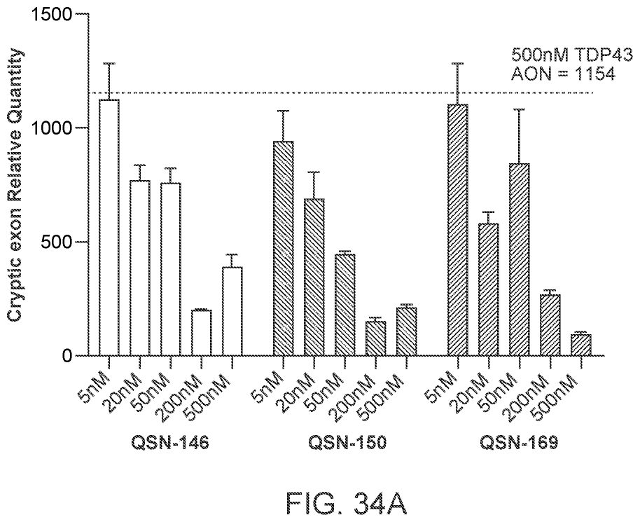

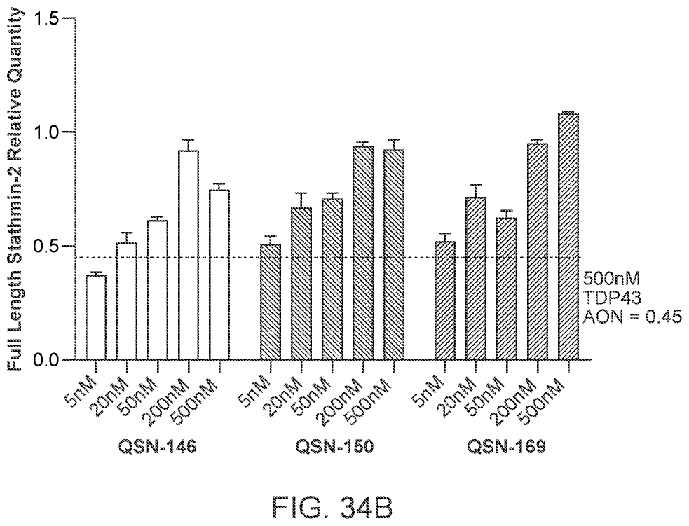

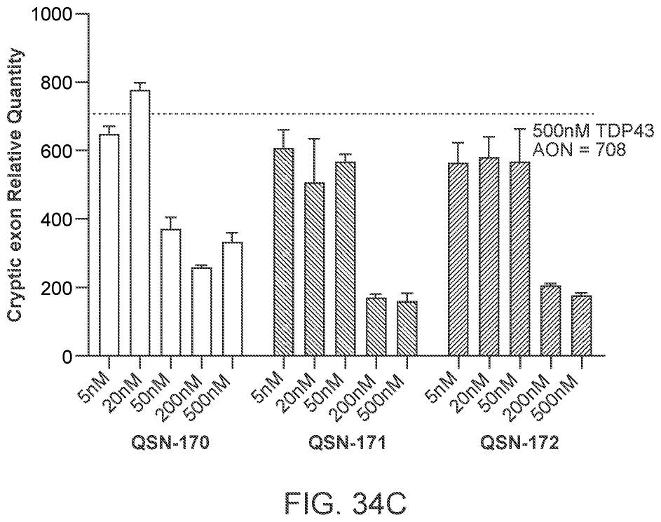

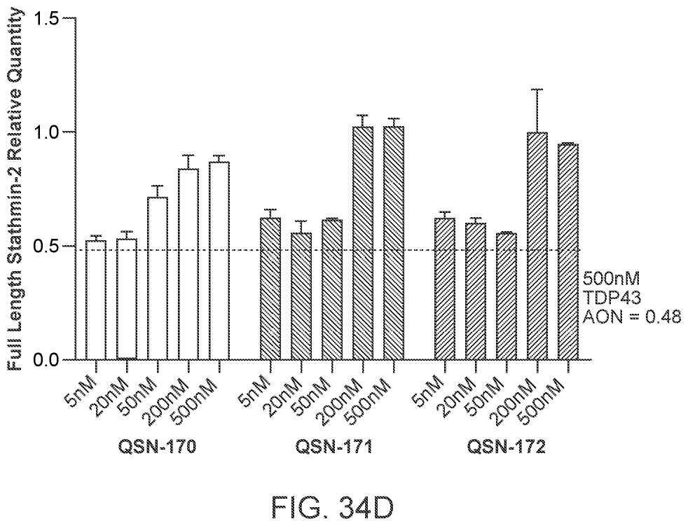

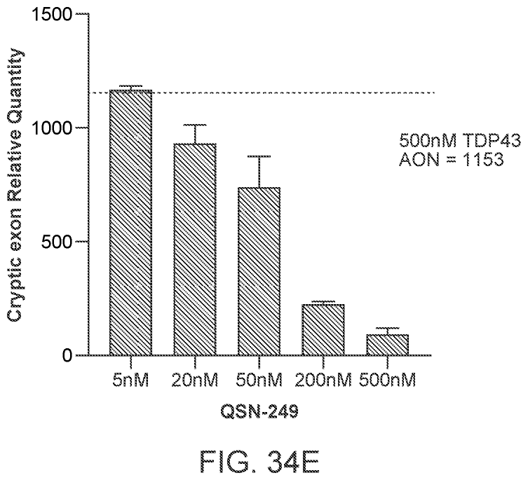

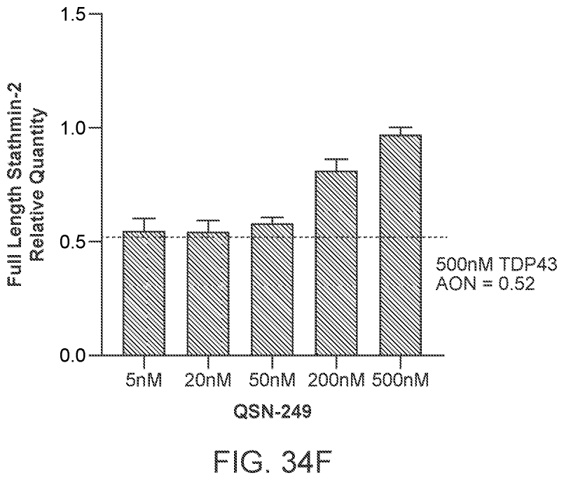

FIGS. 34A, 34C, and 34E are bar graphs showing the results of RT-qPCR analysis of STMN2 transcript with cryptic exon mRNA expression in human motor neurons, which demonstrates reduction of the STMN2 transcript with cryptic exon mRNA levels using different STMN2 AONs. FIGS. 34B, 34D, and 34F are bar graphs showing the results of RT-qPCR analysis of STMN2 full-length mRNA levels, which demonstrates the restoration of the full-length STMN2 transcript using different STMN2 AONs. In particular, the STMN2 AONs tested included: QSN-146 (SEQ ID NO: 146), QSN-150 (SEQ ID NO: 150), QSN-169 (SEQ ID NO: 169), QSN-170 (SEQ ID NO: 170), QSN-171 (SEQ ID NO: 171), QSN-172 (SEQ ID NO: 172), and QSN-249 (SEQ ID NO: 249). The dotted line represents 500 nM TDP43 ASO only level of expression.

Table 18 shows the dose-dependent effect (percentage decrease) of STMN2 AONs on levels of expression of STMN2 with cryptic exon. Additionally, Table 19 shows the dose-dependent effect of STMN2 AONs on restoration of levels of full length STMN2 transcript.

TABLE 18
Dose dependent effect of STMN2 AONs on expression levels of STMN2
with cryptic exon. Values are shown as percentage decrease of expression
levels of STMN2 with cryptic exon relative to corresponding value
derived from 500 nM TDP43 AON treated cells.

| Dose | QSN-146 | QSN-150 | QSN-169 | QSN-170 | QSN-171 | QSN-172 | QSN-249 |
|---|---|---|---|---|---|---|---|
| 5 | 2 | 18 | 4 | 8 | 14 | 20 | −1 |
| 20 | 33 | 40 | 50 | −10 | 29 | 18 | 19 |
| 50 | 34 | 62 | 27 | 47 | 20 | 20 | 35 |
| 200 | 83 | 87 | 77 | 64 | 76 | 71 | 80 |
| 500 | 66 | 82 | 92 | 53 | 77 | 75 | 92 |

TABLE 19
Dose dependent effect of STMN2 AONs on
expression levels of full length STMN2 transcript.

| | QSN-146 | | QSN-150 | | QSN-169 | | QSN-170 | |
|---|---|---|---|---|---|---|---|---|
| | Relative | Percent | Relative | Percent | Relative | Percent | Relative | Percent |
| | Quantity | Increase | Quantity | Increase | Quantity | Increase | Quantity | Increase |
| | (“rescued | of FL | (“rescued | of FL | (“rescued | of FL | (“rescued | of FL |
| Dose | to”) | STMN2 | to”) | STMN2 | to”) | STMN2 | to”) | STMN2 |
| 5 | 0.37 | 82 | 0.51 | 113 | 0.52 | 115 | 0.52 | 108 |
| 20 | 0.52 | 115 | 0.67 | 148 | 0.72 | 159 | 0.53 | 111 |
| 50 | 0.61 | 136 | 0.71 | 157 | 0.62 | 139 | 0.72 | 149 |
| 200 | 0.92 | 204 | 0.94 | 208 | 0.95 | 211 | 0.85 | 177 |
| 500 | 0.75 | 166 | 0.92 | 205 | 1.08 | 240 | 0.87 | 181 |

| | QSN-171 | | QSN-172 | | QSN-249 | |
|---|---|---|---|---|---|---|
| | Relative | Percent | Relative | Percent | Relative | Percent |
| | Quantity | Increase | Quantity | Increase | Quantity | Increase |
| | (“rescued | of FL | (“rescued | of FL | (“rescued | of FL |
| Dose | to”) | STMN2 | to”) | STMN2 | to”) | STMN2 |
| 5 | 0.63 | 131 | 0.62 | 130 | 0.55 | 104 |
| 20 | 0.56 | 116 | 0.60 | 126 | 0.55 | 104 |
| 50 | 0.61 | 127 | 0.55 | 116 | 0.58 | 110 |
| 200 | 1.03 | 214 | 1.00 | 209 | 0.81 | 152 |
| 500 | 1.03 | 214 | 0.95 | 198 | 0.97 | 183 |

### INCORPORATION BY REFERENCE

The entire disclosure of each of the patent documents and scientific articles cited herein is incorporated by reference for all purposes.

### EQUIVALENTS

The disclosure can be embodied in other specific forms without departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A compound comprising an oligonucleotide comprising linked nucleosides with at least a 19 contiguous nucleobase sequence that is at least 90% complementary to an equal length portion of a transcript with at least 90% identity to SEQ ID NO: 944, or to a contiguous 19 to 50 nucleobase portion of SEQ ID NO: 944, wherein at least one nucleoside linkage of the linked nucleosides is a non-natural linkage.

2. An oligonucleotide comprising linked nucleosides with at least a 19 contiguous nucleobase sequence that is at least 90% complementary to an equal length portion of a transcript with at least 90% identity to SEQ ID NO: 944, or to a contiguous 19 to 50 nucleobase portion of SEQ ID NO: 944, wherein at least one nucleoside linkage of the linked nucleosides is a non-natural linkage.

3. The oligonucleotide of claim 1 or 2, wherein the nucleobase sequence comprises a portion of at least 10 contiguous nucleobases that shares at least 90% identity with an equal length portion of any one of SEQ ID NOs: 1-446, SEQ ID NOs: 894-918, SEQ ID NOs: 945-1390, or SEQ ID NOs: 1392-1432.

4. The oligonucleotide of any one of claims 1-3, wherein the nucleobase sequence comprises a portion of at least 11, 12, 13, 14, 15, 16, 17, 18, 19, 20, 21, 22, 23, 24, or 25 contiguous nucleobases that shares at least 90% identity with an equal length portion of any one of SEQ ID NOs: 1-446, SEQ ID NOs: 894-918, SEQ ID NOs: 945-1390, or SEQ ID NOs: 1392-1432.

5. The oligonucleotide of any one of claims 1-3, wherein the nucleobase sequence comprises a portion of at least 10 contiguous nucleobases that shares at least 90% identity with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344.

6. The oligonucleotide of any one of claims 1-5, wherein the nucleobase sequence comprises a portion of at least 11, 12, 13, 14, 15, 16, 17, 18, 19, 20, 21, 22, 23, 24, or 25 contiguous nucleobases that shares at least 90% identity with an equal length portion of any one of SEQ ID NOs: 31, 36, 41, 46, 55, 144, 146, 150, 169, 170, 171, 172, 173, 177, 181, 185, 197, 203, 209, 215, 237, 244, 249, 252, 380, 385, 390, 395, 400, 975, 980, 985, 999, 1088, 1090, 1094, 1113, 1114, 1115, 1116, 1117, 1121, 1125, 1129, 1141, 1147, 1153, 1159, 1181, 1188, 1193, 1196, 1324, 1329, 1334, 1339, or 1344.

7. A compound comprising an oligonucleotide comprising linked nucleosides with at least a 19 contiguous nucleobase sequence, wherein the nucleobase sequence comprises a portion of at least 10 contiguous nucleobases that shares at least 90% identity to an equal length portion of any one of SEQ ID NOs: 894-918 or SEQ ID NOs: 1392-1432.

8. An oligonucleotide comprising linked nucleosides with at least a 19 contiguous nucleobase sequence, wherein the nucleobase sequence comprises a portion of at least 10 contiguous nucleobases that shares at least 90% identity to an equal length portion of any one of SEQ ID NOs: 894-918 or SEQ ID NOs: 1392-1432.

9. The oligonucleotide of claim 7 or 8, wherein the nucleobase sequence comprises a portion of at least 11, 12, 13, 14, 15, 16, 17, 18, 19, 20, 21, 22, 23, 24, or 25 contiguous nucleobases that shares at least 90% identity to of any one of SEQ ID NOs: 894-918 or SEQ ID NOs: 1392-1432.

10. A compound comprising an oligonucleotide comprising linked nucleosides with at least a 19 contiguous nucleobase sequence, wherein the nucleobase sequence comprises a portion of at least 10 contiguous nucleobases that is at least 90% complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944.

11. An oligonucleotide comprising linked nucleosides with a nucleobase sequence with at least a 19 contiguous nucleobase sequence, wherein the nucleobase sequence comprises a portion of at least 10 contiguous nucleobases that is at least 90% complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944.

12. The oligonucleotide of claim 10 or 11, wherein the portion of the nucleobase sequence is 100% complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944.

13. The oligonucleotide of claim 12, wherein the portion of the nucleobase sequence is 100% complementary to an equal length portion of nucleobases within any one of positions 144-164, 144-166, 145-167, 146-166, 146-168, 147-165, or 148-168 of SEQ ID NO: 944.

14. The oligonucleotide of claim 12, wherein the portion of the nucleobase sequence is 100% complementary to an equal length portion of nucleobases within any one of positions 173-191, 173-193, 173-195, 173-197, 175-195, 175-197, 177-197, or 179-197 of SEQ ID NO: 944.

15. The oligonucleotide of claim 12, wherein the portion of the nucleobase sequence is 100% complementary to an equal length portion of nucleobases within any one of positions 185-205, 187-209, 189-209, 185-207, 197-217, 197-219, or 191-209 of SEQ ID NO: 944.

16. The oligonucleotide of claim 12, wherein the portion of the nucleobase sequence is 100% complementary to an equal length portion of nucleobases within any one of positions 237-255, 237-257, 237-259, 239-259, 239-261, 241-261, 237-257, 249-269, 249-271, 252-272, 252-274, or 243-261 of SEQ ID NO: 944.

17. The oligonucleotide of claim 10 or 11, wherein the nucleobase sequence comprises a portion of at least 11, 12, 13, 14, 15, 16, 17, 18, 19, 20, 21, 22, 23, 24, or 25 contiguous nucleobases that is complementary to an equal length portion of nucleobases within any one of positions 121-144, 144-168, 146-170, 150-170, 150-172, 150-174, 169-193, 169-189, 169-191, 170-190, 170-192, 171-191, 171-193, 172-192, 172-194, 170-194, 171-195, 172-196, 173-197, 185-209, 197-221, 237-261, 249-273, 252-276, or 276-300 of SEQ ID NO: 944.

18. The oligonucleotide of claim 10 or 11, wherein the nucleobase sequence comprises a portion of at least 11, 12, 13, 14, 15, 16, 17, 18, 19, 20, 21, 22, 23, 24, or 25 contiguous nucleobases that is complementary to an equal length portion of nucleobases within any one of positions 144-164, 144-166, 145-167, 146-166, 146-168, 147-165, 148-168, 173-191, 173-193, 173-195, 173-197, 175-195, 175-197, 177-197, 179-197, 185-205, 185-207, 197-217, 197-219, 187-209, 189-209, 191-209, 237-255, 237-257, 237-259, 239-259, 239-261, 241-261, 237-257, 249-269, 249-271, 252-272, 252-274, or 243-261 of SEQ ID NO: 944.

19. The oligonucleotide of any one of claims 1-18, wherein the oligonucleotide is 19 and 40 nucleosides in length.

20. The oligonucleotide of any one of the above claims, wherein the oligonucleotide comprises at least one nucleoside linkage selected from the group consisting of a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate (e.g., comprising a phosphorodiamidate morpholino (PMO), 3′ amino ribose, or 5′ amino ribose) linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage, or any combination(s) thereof.

21. The oligonucleotide of any one of the above claims, wherein at least two, three, or four internucleoside linkages of the oligonucleotide are phosphodiester internucleoside linkages.

22. The oligonucleotide of any one of claims 1-20, wherein the oligonucleotide comprises at least two, three, or four modified internucleoside linkages.

23. The oligonucleotide of claim 22, wherein each of the modified internucleoside linkage of the oligonucleotide is independently selected from a phosphorothioate linkage, a phosphoramidate linkage, a phosphoramidothiate linkage, a phosphorodiamidate.

24. The oligonucleotide of claim 22 or 23, wherein all internucleoside linkages of the oligonucleotide are phosphorothioate linkages.

25. The oligonucleotide of claim 23, wherein the phosphorothioate internucleoside linkage is in one of a Rp configuration or a Sp configuration.

26. The oligonucleotide of any one of the preceding claims, wherein the oligonucleotide comprises at least one modified nucleobase.

27. The oligonucleotide of claim 26, wherein the at least one modified nucleobase is 5-methyl cytosine, pseudouridine, or 5-methoxyuridine.

28. The oligonucleotide of any one of the preceding claims, wherein the oligonucleotide comprises at least one modified sugar moiety.

29. The modified oligonucleotide of claim 28, wherein the modified sugar moiety is one of a 2′-OMe modified sugar moiety, bicyclic sugar moiety, 2′-O-(2-methoxyethyl) (2′MOE), 2′-deoxy-2′-fluoro nucleoside, 2′-fluoro-β-D-arabinonucleoside, locked nucleic acid (LNA), constrained ethyl 2′-4′-bridged nucleic acid (cEt), S-cEt, hexitol nucleic acids (HNA), and tricyclic analog (e.g., tcDNA).

30. The oligonucleotide of any one of claims 1-23, wherein the oligonucleotide comprises three linked nucleosides that are linked through phosphodiester internucleoside linkages at the 5′ end and three linked nucleosides that are linked through phosphodiester internucleoside linkages at the 3′ end.

31. The oligonucleotide of any one of claims 1-20 and 22-29, wherein the oligonucleotide comprises one or more 2′-O-(2-methoxyethyl) (2′-MOE) nucleosides that are linked through phosphorothioate internucleoside linkages, optionally wherein all nucleosides in the oligonucleotide comprise modified sugar moiety comprising 2′-MOE; further optionally wherein all cytosine nucleosides in the oligonucleotide comprise modified nucleobase 5-methyl cytosine; and further optionally wherein all internucleoside linkages are phosphorothioate linkages.

32. The oligonucleotide of any one of claims 1-23 and 25-29, wherein the oligonucleotide comprises three linked nucleosides that are linked through phosphorothioate internucleoside linkages at the 5′ end and three linked nucleosides that are linked through phosphorothioate internucleoside linkages at the 3′ end.

33. The oligonucleotide of claim 32, wherein the oligonucleotide comprises five linked nucleosides that are linked through phosphodiester internucleoside linkages.

34. The oligonucleotide of claim 33, wherein the each of the five linked nucleosides are 2′-O-(2-methoxyethyl) (2′-MOE) nucleosides.

35. The oligonucleotide of claim 33 or 34, wherein each of the linked nucleosides of the oligonucleotide are 2′-O-(2-methoxyethyl) (2′-MOE) nucleosides.

36. The oligonucleotide of any one of claims 1-35, wherein the oligonucleotide exhibits at least a 30%, 40%, 50%, 60%, 70%, 80%, or 90% increase of full length STMN2 transcript or STMN2 protein, optionally wherein the increase is in comparison to a level prior to exposing a neuron to the oligonucleotide.

37. The oligonucleotide of any one of claims 1-36, wherein the oligonucleotide exhibits at least a 100% increase of full length STMN2 transcript or STMN2 protein, optionally wherein the increase is in comparison to a level prior to exposing a neuron to the oligonucleotide.

38. The oligonucleotide of any one of claims 1-37, wherein the oligonucleotide exhibits at least a 200% increase of full length STMN2 transcript or STMN2 protein, optionally wherein the increase is in comparison to a level prior to exposing a neuron to the oligonucleotide.

39. The oligonucleotide of any one of claims 1-38, wherein the oligonucleotide exhibits at least a 300% increase of full length STMN2 transcript or STMN2 protein, optionally wherein the increase is in comparison to a level prior to exposing a neuron to the oligonucleotide.

40. The oligonucleotide of any one of claims 1-39, wherein the oligonucleotide exhibits at least a 400% increase of full length STMN2 transcript or STMN2 protein, optionally wherein the increase is in comparison to a level prior to exposing a neuron to the oligonucleotide.

41. The oligonucleotide of any one of claims 36-40, wherein increase of the full length STMN2 protein is measured in comparison to a reduced level of full length STMN2 protein achieved using a TDP43 antisense oligonucleotide.

42. The oligonucleotide of any one of claims 1-35, wherein the oligonucleotide exhibits at least a 10%, 20%, 30%, 40%, 50%, 60%, 70%, 80%, 90%, or 100% rescue of full length STMN2 transcript or STMN2 protein, optionally wherein the increase is in comparison to a level prior to exposing a neuron to the oligonucleotide.

43. The oligonucleotide of any one of claims 1-35 and 42, wherein the oligonucleotide exhibits at least a 50%, 60%, 70%, 80%, or 90% reduction of the STMN2 transcript with the cryptic exon.

44. A pharmaceutical composition comprising one or more of the oligonucleotides of any one of claims 1-43, or a pharmaceutically acceptable salt thereof, and a pharmaceutically acceptable excipient.

45. A method of treating a neurological disease and/or a neuropathy in a patient in need thereof, the method comprising administering to the patient an oligonucleotide of any one of claims 1-43 or a pharmaceutically acceptable salt thereof, or a pharmaceutical composition of claim 44.

46. The method of claim 45, wherein the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD).

47. The method of claim 45, wherein the neuropathy is chemotherapy induced neuropathy.

48. A method of restoring axonal outgrowth and/or regeneration of a motor neuron, the method comprising exposing the motor neuron to an oligonucleotide of any one of claims 1-43 or a pharmaceutically acceptable salt thereof, or a pharmaceutical composition of claim 44.

49. A method of increasing, promoting, stabilizing, or maintaining STMN2 expression and/or function in a neuron, the method comprising exposing the neuron to an oligonucleotide of any one of claims 1-43 or a pharmaceutically acceptable salt thereof, or a pharmaceutical composition of claim 44.

50. The method of claim 48 or 49, wherein the neuron is a neuron of a patient in need of treatment of a neurological disease and/or a neuropathy.

51. The method of claim 50, wherein the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD).

52. The method of claim 50, wherein the neuropathy is chemotherapy induced neuropathy.

53. The method of any one of claims 48-52, wherein the exposing is performed in vivo or ex vivo.

54. The method of any one of claims 48-52, wherein the exposing comprises administering the oligonucleotide to a patient determined to have a transcript comprising a cryptic exon sequence of SEQ ID NO: 447.

55. The method of any one of claims 45-54, wherein the oligonucleotide is administered topically, parenterally, intrathecally, intracisternally, orally, rectally, buccally, sublingually, vaginally, pulmonarily, intratracheally, intranasally, intralesionally, transdermally, or intraduodenally.

56. The method of claim 54, wherein the oligonucleotide is administered orally.

57. The method of any one of claims 45-54, wherein a therapeutically effective amount of the oligonucleotide is administered intrathecally or intracisternally.

58. The method of any one of claim 45-46 or 50-57, wherein the patient is a human.

59. The pharmaceutical composition of claim 44, wherein the pharmaceutical composition is suitable for topical, intrathecal, intracisternal, parenteral (e.g., subcutaneous, intramuscular, intradermal, intraduodenal, or intravenous), intralesional, oral, pulmonary, intratracheal, intranasal, transdermal, rectal, buccal, sublingual, vaginal, or intraduodenal administration.

60. A use of an oligonucleotide of any one of claims 1-43 or a pharmaceutically acceptable salt thereof, or a pharmaceutical composition of claim 44 in the manufacture of a medicament for the treatment of neurological disease or a neuropathy.

61. The use of claim 60, wherein the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD).

62. The use of claim 60, wherein the neuropathy is chemotherapy induced neuropathy.

63. A method of treating a neurological disease or a neuropathy in a patient in need thereof, the method comprising administering to a patient in need thereof a therapeutically effective amount of an oligonucleotide of any one of claims 1-43 or a pharmaceutically acceptable salt thereof, or a pharmaceutical composition of claim 44.

64. The method of claim 63, wherein the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD).

65. The method of claim 63, wherein the neuropathy is chemotherapy induced neuropathy.

66. The method of any one of claims 63-65, wherein the oligonucleotide or the pharmaceutical composition is administered topically, parenterally (e.g., subcutaneous, intramuscular, intradermal, intraduodenal, or intravenous), intralesionally, orally, pulmonarily, rectally, buccally, sublingually, vaginally, intratracheally, intranasally, intracisternally, intrathecally, transdermally, or intraduodenally.

67. The method of any one of claims 63-65, wherein the oligonucleotide or the pharmaceutical composition is administered intrathecally or intracisternally.

68. The method of any one of claims 63-67, wherein a therapeutically effective amount of the oligonucleotide or the pharmaceutical composition is administered intrathecally or intracisternally.

69. The method of any one of claims 63-68, wherein the patient is human.

70. An oligonucleotide of any one of claims 1-43, or a pharmaceutically acceptable salt thereof, for use as a medicament in the treatment of a neurological disease or a neuropathy.

71. An oligonucleotide of any one of claims 1-43, or a pharmaceutically acceptable salt thereof, for use in the treatment of a neurological disease or a neuropathy.

72. The oligonucleotide for use of claim 70 or 71, wherein said neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD).

73. The oligonucleotide for use of claim 70 or 71, wherein the neuropathy is chemotherapy induced neuropathy.

74. An oligonucleotide comprising linked nucleosides with a nucleobase sequence of any one of SEQ ID NOs: 1-446, SEQ ID NOs: 894-918, SEQ ID NOs: 945-1390, or SEQ ID NOs: 1392-1432, or a pharmaceutically acceptable salt thereof;
wherein oligonucleotide comprises at least one nucleoside linkage selected from the group consisting of: a phosphodiester linkage, a phosphorothioate linkage, an alkyl phosphate linkage, an alkylphosphonate linkage, a 3-methoxypropyl phosphonate linkage, a phosphorodithioate linkage, a phosphotriester linkage, a methylphosphonate linkage, an aminoalkylphosphotriester linkage, an alkylene phosphonate linkage, a phosphinate linkage, a phosphoramidate linkage, a phosphoramidothiate linkage, a phosphorodiamidate linkage, an aminoalkylphosphoramidate linkage, a thiophosphoramidate linkage, a thionoalkylphosphonate linkage, a thionoalkylphosphotriester linkage, a thiophosphate linkage, a selenophosphate linkage, and a boranophosphate linkage; and/or
wherein at least one nucleoside of the linked nucleosides is substituted with a component selected from the group consisting of a 2′-O-(2-methoxyethyl) nucleoside (2′-O-methoxyethylribonucleosides (2′-MOE)), a 2′-O-methyl nucleoside, a 2′-deoxy-2′-fluoro nucleoside, a 2′-fluoro-β-D-arabinonucleoside, a locked nucleic acid (LNA), constrained methoxyethyl (cM0E), constrained ethyl (cET), and a peptide nucleic acid (PNA).

75. The oligonucleotide of claim 74, wherein at least one internucleoside linkage of the oligonucleotide is a phosphorothioate linkage.

76. The oligonucleotide of claim 74 or 75, wherein the oligonucleotide comprises three linked nucleosides that are linked through phosphodiester internucleoside linkages at the 5′ end and three linked nucleosides that are linked through phosphodiester internucleoside linkages at the 3′ end.

77. The oligonucleotide of any one of claims 74-76, wherein the oligonucleotide comprises one or more 2′-O-(2-methoxyethyl) nucleosides that are linked through phosphorothioate internucleoside linkages.

78. The oligonucleotide of claim 74 or 75, wherein the oligonucleotide comprises five linked nucleosides that are linked through phosphodiester internucleoside linkages.

79. The oligonucleotide of claim 78, wherein each of the five linked nucleosides are 2′-O-(2-methoxyethyl) (2′-MOE) nucleosides.

80. The oligonucleotide of any one of claims 74-79, wherein each of the linked nucleosides of the oligonucleotide are 2′-O-(2-methoxyethyl) (2′-MOE) nucleosides.

81. The oligonucleotide of claim 74 or 75, wherein all internucleoside linkages of the oligonucleotide are phosphorothioate linkages, optionally wherein each of the linked nucleosides of the oligonucleotide are 2′-O-(2-methoxyethyl) (2′-MOE) nucleosides, further optionally wherein the oligonucleotide comprises at least one 5-methyl cytosine modified nucleobase.

82. A pharmaceutical composition comprising the oligonucleotide of any one of claims 73-81, or a pharmaceutically acceptable salt thereof, and a pharmaceutically acceptable excipient.

83. An oligonucleotide of any one of claims 1-43 or a pharmaceutically acceptable salt thereof capable of increasing, restoring, or stabilizing expression of the STMN2 mRNA capable of translation of a functional STMN2 and/or activity and/or function of STMN2 protein in a cell or a human patient suffering from a neurological disease or disorder, wherein the level of increase, restoration, or stabilization of expression and/or activity and/or function is sufficient for use of the oligonucleotide as a medicament for the treatment of neurological disease or disorder.

84. The oligonucleotide of any one of claims 1-43 comprising one or more chiral centers and/or double bonds.

85. The oligonucleotide of claim 84, wherein the oligonucleotide exist as stereoisomers selected from geometric isomers, enantiomers, and diastereomers.

86. A method of treating a neurological disease and/or a neuropathy in a patient in need thereof, the method comprising administering to a patient in need thereof a therapeutically effective amount of an oligonucleotide of any one of claims 1-43 or a pharmaceutically acceptable salt thereof, or a pharmaceutical composition of claim 44, in combination with a second therapeutic agent selected from Riluzole (Rilutek), Edaravone (Radicava), rivastigmine, donepezil, galantamine, selective serotonin reuptake inhibitor, antipsychotic agents, cholinesterase inhibitors, memantine, benzodiazepine antianxiety drugs, AMX0035 (ELYBRIO), ZILUCOPLAN (RA101495), dual AON intrathecal administration (e.g., BIIB067, BIIB078), BIIB100, levodopa/carbidopa, dopaminergic agents (e.g., ropinirole, pramipexole, rotigotine), medroxyprogesterone, KCNQ2/KCNQ3 openers, anticonvulsants and psychostimulant agents, and/or a therapy (e.g., selected from breathing care, physical therapy, occupational therapy, speech therapy, nutritional support), for treating said neurologic disease.

87. The method of claim 86, wherein the neurological disease is selected from the group consisting of amyotrophic lateral sclerosis (ALS), frontotemporal dementia (FTD), Alzheimer's disease (AD), Parkinson's disease (PD), Huntington's disease, brachial plexus injuries, peripheral nerve injuries, progressive supranuclear palsy (PSP), brain trauma, spinal cord injury, and corticobasal degeneration (CBD).

88. The method of claim 86, wherein the neuropathy is chemotherapy induced neuropathy.

89. The method of any one of claims 45-58, 63-69, and 86-88, wherein patient for treatment is identified by measuring the presence or level of expression of neurofilament light (NEFL), neurofilament heavy (NEFH), phosphorylated neurofilament heavy chain (pNFH), TDP-43, or p75^{ECD} in the plasma, the spinal cord fluid, the cerebrospinal fluid, the extracellular vesicles (for example, CSF exosomes), the blood, the urine, the lymphatic fluid, fecal matter, or a tissue of the patient.

90. The method of claim 89, wherein the patient for treatment is identified by measuring phosphorylated neurofilament heavy chain (pNFH) in cerebrospinal fluid (CSF).

91. The method of claim 90, wherein the pNFH in the CSF of the patient is used to predict disease status and survival in C9ORF72-associated amyotrophic lateral sclerosis (c9ALS) patients after initial administration and/or during on-going treatment.
